(19) United States
(12) Patent Application Publication
(10) Pub. No.: US 2019/0071416 A1
(43) Pub. Date: Mar. 7, 2019

(54) COMPOUNDS FOR TREATMENT OF CANCER AND EPIGENETICS

(71) Applicant: Agency For Science, Technology and Research, Singapore (SG)

(72) Inventors: Klement Jihao Foo, Singapore (SG); Anders Poulsen, Singapore (SG); Thomas Hugo Keller, Singapore (SG); Si Si Liew, Singapore (SG); Cheng San Brian Chia, Singapore (SG); Jin Yan Melgious Ang, Singapore (SG); Chuhul Huang, Singapore (SG)

(21) Appl. No.: 15/767,129

(22) PCT Filed: Oct. 10, 2016
(86) PCT No.: PCT/SG2016/050499

§ 371 Date: Apr. 9, 2018

(30) Foreign Application Priority Data
Oct. 9, 2015 (SG) 10201508413Q

(51) Int. Cl. C07D 401/06; A61P 35/00; C07D 401/14; C07D 413/14; C07D 417/14; C07D 471/08; C07D 405/14

(52) U.S. Cl. CPC C07D 401/06; A61P 35/00; C07D 401/14; C07D 405/14; C07D 417/14; C07D 471/08; C07D 413/14

(57) ABSTRACT

Compounds For Inhibition Of Cancer and Epigenesis. The present invention relates to quinolines and 5,6,7,8-tetrahydroacridines of the formula (I) wherein Z¹, Z², X, R¹ to R⁸ and Y are defined as described in the specification, or a pharmaceutically acceptable form or prodrug thereof, that are inhibitors of methyl transferases such as protein lysine methyltransferases and more particularly SMYD3. The present invention also relates to the methods for their preparation, pharmaceutical compositions containing these compounds and uses of these compounds in the treatment of disorders/conditions/diseases involving, relating to or associated with enzymes having methyl transferase activities/functions and/or via unspecified/multi-targeted mechanisms.

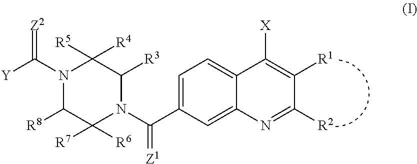

### TECHNICAL FIELD

The present invention generally relates to quinolines and 5,6,7,8-tetrahydroacridine derivatives, methods for their preparation, pharmaceutical compositions containing these compounds and uses of these compounds in the treatment of disorders/conditions/diseases involving, relating to or associated with enzymes having methyltransferase activities.

### BACKGROUND ART

The genomes of eukaryotic organisms are tightly packaged into chromatin, which forms the structural basis of nuclear processes associated with genetic activity. Nucleosome is the smallest structural unit of chromatin, in which 146 base pairs of DNA are wrapped around an octamer of core histones. The histones are subjected to several post-translational covalent modifications and this plays a critical role in controlling gene transcription within cells. Among the various histone modifications, methylation of lysine residue seems to play a particularly important role in control of gene transcription programs (Arrowsmith, C. H., et al. Epigenetic protein families—a new frontier for drug discovery. Nat. Rev. Drug Discov. 2012, 11, 384-400). These modifications are catalyzed by a class of group-transfer enzymes known as the protein lysine methyltransferases (PKMT).

There are more than 100 protein methyltransferases that are encoded by the human genome and they are subdivided into five subfamilies based on their primary sequence. Misregulation of these proteins, through overexpression, deletion or chromosomal translocations has been implicated in many types of human cancers. PKMTs contain an evolutionarily conserved SET (Su(var), E(z) and Trithorax) domain that is involved in catalyzing the transfer of the methyl group from the cofactor S-adenosyl-L-methionine (SAM) to lysine residues of histone and non-histone substrates, leading to lysine mono-, di-, and/or trimethylation. Histone lysine methylation has been increasingly recognized as a major epigenetic gene regulation mechanism in eukaryotic cells (Copeland, R. A., Molecular pathways: protein methyltransferases in cancer. Clin. Cancer Res. 2013, 19, 6344-6350).

SMYD3 is a histone methyltransferase and is overexpressed in several cancers including breast, gastric, pancreatic, colorectal, lung cancer and hepatocellular carcinoma. It tri-methylates histone H3 at lysine 4 (H3K4mc3), a mark associated with gene activation. SMYD3 is part of the SMYD family (SET/MYND) of proteins which contains five members carrying a SET domain and a MYND type of zinc finger. The up-regulation of the SMYD3 promotes proliferation of HCC (hepatocellular carcinoma) via increasing H3 lysine 4 methylation, and a subsequent activation of downstream genes, including Nkx2.8 gene which is frequently up-regulated in human HCC (Hamamoto, R., et al. SMYD3 encodes a histone methyltransferase involved in the proliferation of cancer cells. Nat. Cell Biol. 2004, 6, 731-740).

SMYD3 also catalyzes histone H4 methylation at lysine 5 (H4K5me). This novel histone methylation mark is detected in diverse cell types and its formation is attenuated by depletion of SMYD3 protein. It has been shown that the catalytic activity of SMYD3 is required for the anchorage-independent growth of cancer cells. Thus, SMYD3, via H4K5 methylation, provides another link between chromatin dynamics and neoplastic disease (Van Eller, et al. Smyd3 regulates cancer cell phenotypes and catalyzes histone H4 lysine 5 methylation. Epigenetics 2012, 7, 340-343).

SMYD3 modulates myostatin and c-Met transcription in primary skeletal muscle cells and C2C12 myogenic cells. It does this by targeting the myostatin and c-Met genes and participates in the recruitment of the bromodomain protein BRD4 to their regulatory regions through protein—protein interaction. By recruiting BRD4, SMYD3 favors chromatin engagement of the pause—release factor p-TEFb (positive transcription elongation factor) and elongation of Ser2-phosphorylated RNA polymerase II (PolIISer2P). SMYD3 is also known to methylate other substrates such as RB1 protein (CA2613322 A1) and VEGFR1 (US8354223 B2).

It has been shown in mouse models of K-Ras-driven cancer, that SMYD3 acts in the cytoplasm of cancer cells, methylating a lysine residue (K260) on MAP3K2, a kinase enzyme that is associated with the activation of MEK-ERK mitogen-activated protein-kinase pathway (Mazur, P., et.al. SMYD3 links lysine methylation of MAP3K2 to Ras-driven cancer. Nature 2014, 510, 283-287).

Early 2015, a SMYD3 inhibitor BCI-121 was reported by Peserico, A., et. al. (A SMYD3 small-molecule inhibitor impairing cancer cell growth. J. Cell Physiol. 2015, 230, 2447-2460). There was no reported biochemical assay data in the publication, and this inhibitor was found to be inactive against SMYD3 using the biochemical assay disclosed herein (Examples Section: Comparative Example 1).

Another prior art known includes tetrahydroacridine derivatives (WO 2011/086178) found to act against against a different target ubiquitin specific protease 7. The compounds disclosed in WO2011/086178 were found to act against a different target ubiquitin specific protease 7, but were not found to be active against SMYD3.

The following compounds are also known from Scifinder: ethyl 4-(9-chloro-5,6,7,8-tetrahydroacridine-3-carbonyl)piperazine-1-carboxylate and ethyl 4-(9-chloro-2,3-dihydro-1H-cyclopenta[b]quinoline-6-carbonyl)piperazine-1-carboxylate . Ethyl 4-(9-chloro-5,6,7,8-tetrahydroacridine-3-carbonyl)piperazine-1-carboxylate was found to be moderately active against SMYD3, but suffered from poor metabolic stability due to high metabolic clearance in the human/mouse liver microsomes stability test. In addition, ethyl 4-(9-chloro-5,6,7,8-tetrahydroacridine-3-carbonyl)piperazine-1-carboxylate was found to have poor target engagement compared to a more advanced compound disclosed herein.

Recently, another small molecule inhibitor of SMYD3 was reported (Mitchell, L. H., et. al. Novel Oxindole Sulfonamides and Sulfamides: EPZ031686, the First Orally Bioavailable Small Molecule SMYD3 Inhibitor. ACS Med. Chem. Lett. 2015, ASAP). Although the reported molecule was active against SMYD3, no anti-proliferative cellular activity was disclosed. Moreover, the structure of the inhibitor is not related to the compounds in this application.

There is therefore an urgent need to provide novel compounds that overcome, or at least ameliorates, one or more of the disadvantages of the effects of protein lysine methyltransferases such as SMYD3 described above. There is also a need to provide a pharmaceutical composition comprising the compound, methods for treating diseases using the compound and a method for synthesizing the compound.

### SUMMARY

In a first aspect, there is provided a compound having the following Formula (I);

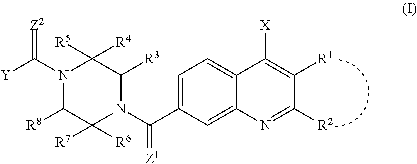

wherein

Z¹ and Z² are independently selected from O, S or NH;

X is a halogen;

R¹ and R² are independently selected from the group consisting of a bond, H, halogen, cyano, optionally substituted alkyl, optionally substituted alkene, optionally substituted alkyne, optionally substituted alkoxy, optionally substituted amino, optionally substituted cycloalkyl, optionally substituted heterocycloalkyl , optionally substituted aryl and optionally substituted heteroaryl;

and wherein R¹ and R² may optionally be taken together to form an optionally substituted alkylene bridge wherein one or two alkylene units may be replaced with O, NH or S;

and wherein R¹ and R² may optionally form an optionally substituted aryl or optionally substituted heteroaryl together with the ring atoms that they are bonded to;

R³, R⁴, R⁵, R⁶, R⁷ and R⁸ are independently absent, or selected from the group consisting of a bond, hydrogen, halogen, optionally substituted alkyl, optionally substituted alkene, optionally substituted alkyne, optionally substituted alkoxy, optionally substituted amino, optionally substituted acyl, optionally substituted cycloalkyl, optionally substituted heterocycloalkyl, optionally substituted aryl and optionally substituted heteroaryl;

wherein any two of R³, R⁴, R⁵, R⁶, R⁷ or R⁸ may be taken together to form an optionally substituted cycloalkyl or an optionally substituted alkylene bridge or an optionally substituted alkylene bridge wherein one or two alkylene units may be replaced with O, NH or S;

Y is selected from R⁹, OR⁹ or NHR⁹, wherein R⁹ is an optionally substituted C₃ to C₁₀ alkyl, optionally substituted C₃ to C₁₀ alkenyl, optionally substituted C₃ to C₁₀ alkynyl, optionally substituted C₃ to C₇ cycloalkyl, optionally substituted C₂ to C₁₀ haloalkyl, a substituted 5-membered heteroaryl comprising two to three heteroatoms selected from N, O or S or a C₁ to C₂ alkyl substituted with an optionally substituted 5-membered heterocycloalkyl comprising one to two heteroatoms selected from N, O or S;

or a pharmaceutically acceptable form or prodrug thereof.

In an embodiment, there is provided the compound as defined above, having the following Formula (III):

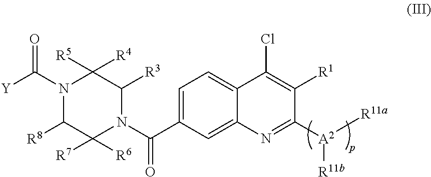

wherein R¹ and R^{11a} are independently selected from the group consisting of H, halogen, cyano, optionally substituted alkyl, optionally substituted alkene, optionally substituted alkyne, optionally substituted alkoxy, optionally substituted amino, optionally substituted cycloalkyl, optionally substituted heterocycloalkyl, optionally substituted aryl and optionally substituted heteroaryl;

R^{11b} may be absent, H or optionally substituted alkyl;

A² is selected from CH, N, O or S; and

p is an integer selected from 0, 1 or 2.

In another embodiment, there is provided the compound as defined above, having the following Formula (IV):

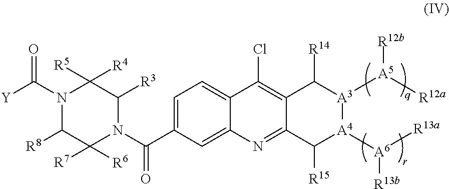

wherein

A³ and A⁴ are independently selected from CH or N;

A⁵ and A⁶ are independently selected from CH, N, O or S;

_{R}^{12a}, R^{13a}, R¹⁴ and R¹⁵ are independently selected from the group consisting of hydrogen, halogen, optionally substituted alkyl, optionally substituted alkene, optionally substituted alkyne, optionally substituted alkoxy, optionally substituted amino, optionally substituted acyl, optionally substituted cycloalkyl, optionally substituted heterocycloalkyl, optionally substituted aryl and optionally substituted heteroaryl;

R^{12b} and R^{13b} are independently absent, H or an optionally substituted alkyl; and

q and r are independently integers selected from 0, 1 or 2.

Advantageously, the compound as defined above is an inhibitor of protein lysine methyltransferases (PKMT) such as SMYD3. SMYD3 is an attractive target for drug discovery due to its role in epigenetic regulation and crucial cell signalling pathways. Advantageously, a small molecule inhibitor of SMYD3 as defined above may be useful for the treatment of cancers with elevated SMYD3 expression such as hepatocellular carcinoma (HCC).

Advantageously, the compounds as defined above have a unique potency profile against the target protein. The compounds may be modified to have different potencies against different targets for a variety of indications or applications. More advantageously, the compound is a small molecule inhibitor Small molecule inhibitors, unlike macromolecules such as polymers, proteins and DNA, may be less toxic and have fewer occurrences of adverse drug effects while maintaining a high level of activity.

Further advantageously, the tested compounds as defined above have a siginificantly higher potency against the target protein compared to conventionally known compounds. The compound as defined above has a significantly higher potency in inhibiting SMYD3.

In a second aspect, there is provided a pharmaceutical composition comprising a compound as defined above, or a pharmaceutically acceptable form or prodrug thereof, and a pharmaceutically acceptable excipient.

In a third aspect, there is provided a method of inhibiting SMYD3 in a cell comprising administering to a cell the compound as defined above, or a pharmaceutically acceptable form or prodrug thereof, or a composition as defined above.

In a fourth aspect, there is provided a method of treating a SMYD3-related disorder comprising administering to a subject in need of treatment a compound as defined above, or a pharmaceutically acceptable form or prodrug thereof, or a composition as defined above.

In a fifth aspect, there is provided a use of a compound as defined above, or a pharmaceutically acceptable form or prodrug thereof, or a composition as defined above, in the manufacture of a medicament for treatment of a SMYD3-related disorder.

In a sixth aspect, there is provided a compound as defined above, or a pharmaceutically acceptable form or prodrug thereof, or a composition as defined above, for use in the treatment of a SMYD3-related disorder.

Advantageously, the compounds as defined above have demonstrated inhibitory activities against the methyl transferase activity of SMYD3 enzyme and anti-proliferative activities against a variety of human tumor cell lines. The compound as defined above may demonstrate good drug-like properties, that is, in vitro metabolic stability, solubility and desirable lipophilicity. More advantageously, the compounds inhibit methyltransferase activity of SMYD3 in an MTase assay using MAP3K2 as a peptide substrate. Further advantageously, the compounds show antiproliferative activity. Further advantageously, the compounds inhibit SMYD3 mediated methylation of MAP3K2 and inhibit anchorage independent growth in human cancer cells.

In an eight aspect, there is provided a process for synthesizing the compound as defined above having the following Formula (III), comprising the steps of:

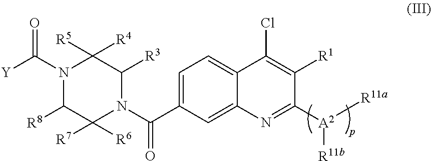

(a) contacting an optionally substituted aminobenzoate ester with a compound having the following Formula (Va) to form a cyclized product;

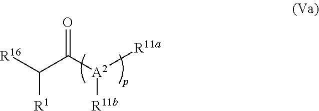

wherein R¹⁶ is selected from the group consisting of H, methyl, COOMe and COOEt;

(b) selectively displacing at least one ketone of the cyclized product of step (a) with a halogen;

(c) selectively hydrolyzing the ester of the cyclized product of step (a) to a carboxylic acid and selectively functionalizing the carboxylic acid with a group having the following formula (VI) under reaction conditions to form the compound of formula (III);

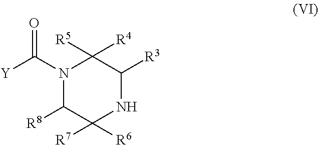

wherein step (b) and (c) may be performed simultaneously, sequentially or in any order.

In a ninth aspect, there is provided a process for synthesizing the compound as defined above having the following Formula (III), wherein R¹ is hydrogen; comprising the steps of:

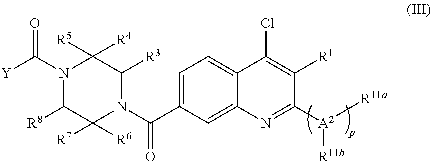

(a) contacting an optionally substituted aminobenzoate ester with a compound having the following Formula (Vb) and phosphorus oxychloride to form a halogenated cyclized product;

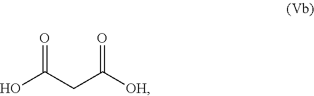

(b) selectively hydrolyzing the ester of the cyclized product of step a) to a carboxylic acid and selectively functionalizing the carboxylic acid with a group having the following formula (VI) under reaction conditions to form an amide; and

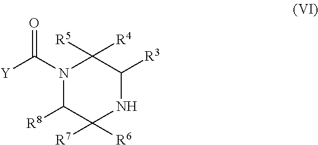

(c) selectively functionalizing at least one halogen of the halogenated cyclized product of step (a) with a group having the following formula (VII) under reaction conditions to form the compound of formula (III);

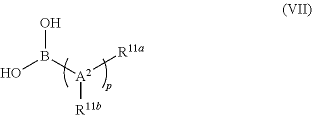

wherein step (b) and (c) may be performed simultaneously, sequentially or in any order.

In a tenth aspect, there is provided a process for synthesizing the compound as defined above having the following formula (IV), comprising the steps of;

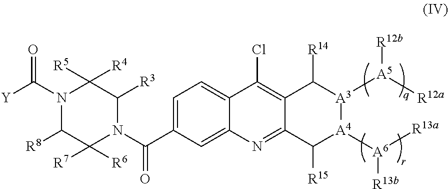

(a) contacting an amino substituted terephthalic acid or an ester thereof; with an optionally substituted cyclic ketone having the following Formula (VIII) to form a cyclized product;

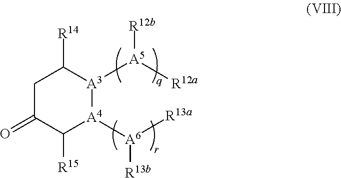

(b) selectively displacing at least one ketone of the cyclized product of step (a) with a halogen;

(c) optionally selectively hydrolyzing the ester of the cyclized product of step a) to a carboxylic acid; and

(d) selectively functionalizing the carboxylic acid of the cyclized product of step (a) or (c) with a group having the following formula (VI) under reaction conditions to form the compound of formula (IV);

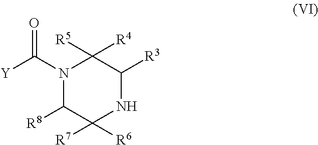

wherein step (b), (c) and (d) may be performed simultaneously, sequentially or in any order.

### Definitions

In this specification a number of terms are used which are well known to a skilled addressee. Nevertheless for the purposes of clarity a number of terms will be defined. The following words and terms used herein shall have the meaning indicated:

In the definitions of a number of substituents below it is stated that “the group may be a terminal group or a bridging group”. This is to signify that the use of the term is intended to encompass the situation where the group is a linker between two other portions of the molecule as well as where it is a terminal moiety. Using the term alkyl as an example, some publications would use the term “alkylene” for a bridging group and hence in these other publications there is a distinction between the terms “alkyl” (terminal group) and “alkylene” (bridging group). In the present application no such distinction is made and most groups may be either a bridging group or a terminal group.

“Acyl” means an R-C(═O)— group in which the R group may be an optionally substituted alkyl, optionally substituted cycloalkyl, optionally substituted heterocycloalkyl, optionally substituted aryl or optionally substituted heteroaryl group as defined herein. Examples of acyl include acetyl, benzoyl and amino acid derived aminoacyl. The group may be a terminal group or a bridging group. If the group is a terminal group it is bonded to the remainder of the molecule through the carbonyl carbon.

“Acylamino” means an R-C(═O)—NH— group in which the R group may be an alkyl, cycloalkyl, heterocycloalkyl; aryl or heteroaryl group as defined herein. The group may be a terminal group or a bridging group. If the group is a terminal group it is bonded to the remainder of the molecule through the nitrogen atom.

“Aliphatic” means non-aromatic, open chain, straight or branched organic compounds.

“Alkenyl” as a group or part of a group denotes an aliphatic hydrocarbon group containing at least one carbon-carbon double bond and which may be straight or branched preferably having 2-12 carbon atoms, more preferably 2-10 carbon atoms, most preferably 2-6 carbon atoms, in the normal chain. The group may contain a plurality of double bonds in the normal chain and the orientation about each is independently E or Z. Exemplary alkenyl groups include, but are not limited to, ethenyl, propenyl, butenyl, pentenyl, hexenyl, heptenyl, octenyl and nonenyl. The group may be a terminal group or a bridging group.

“Alkenyloxy” refers to an alkenyl-O— group in which alkenyl is as defined herein. Preferred alkenyloxy groups are C₁-C₆ alkenyloxy groups. The group may be a terminal group or a bridging group. If the group is a terminal group it is bonded to the remainder of the molecule through the oxygen atom.

“Alkyl” or “alkylene” as a group or part of a group refers to a straight or branched aliphatic hydrocarbon group, preferably a C₁-C₁₂ alkyl, more preferably a C₁-C₁₀ alkyl, most preferably C₁-C₆ unless otherwise noted. Examples of suitable straight and branched C₁-C₆ alkyl substituents include methyl, ethyl, n-propyl, 2-propyl, n-butyl, sec-butyl, t-butyl, hexyl, and the like. The group may be a terminal group or a bridging group.

“Alkylamino” includes both mono-alkylamino and dialkylamino, unless specified. “Mono-alkylamino” means an Alkyl-NH— group, in which alkyl is as defined herein. “Dialkylamino” means a (alkyl)₂N— group, in which each alkyl may be the same or different and are each as defined herein for alkyl. The alkyl group is preferably a C₁-C₆ alkyl group. The group may be a terminal group or a bridging group. If the group is a terminal group it is bonded to the remainder of the molecule through the nitrogen atom.

“Alkylaminocarbonyl” refers to a group of the formula (Alkyl)ₓ(H)_{y}NC(═O)— in which alkyl is as defined herein, x is 1 or 2, and the sum of X+Y=2. The group may be a terminal group or a bridging group. If the group is a terminal group it is bonded to the remainder of the molecule through the carbonyl carbon.

“Alkyloxy” refers to an alkyl-O— group in which alkyl is as defined herein. Preferably the alkyloxy is a C₁-C₆ alkyloxy. Examples include, but are not limited to, methoxy and ethoxy. The group may be a terminal group or a bridging group. The term alkyloxy may be used interchangeably with the term “alkoxy”.

“Alkyloxyalkyl” refers to an alkyloxy-alkyl-group in which the alkyloxy and alkyl moieties are as defined herein. The group may be a terminal group or a bridging group. If the group is a terminal group it is bonded to the remainder of the molecule through the alkyl group.

“Alkyloxyaryl” refers to an alkyloxy-aryl-group in which the alkyloxy and aryl moieties are as defined herein. The group may be a terminal group or a bridging group. If the group is a terminal group it is bonded to the remainder of the molecule through the aryl group.

“Alkyloxycarbonyl” refers to an alkyl-O—C(═O)— group in which alkyl is as defined herein. The alkyl group is preferably a C₁-C₆ alkyl group. Examples include, but are not limited to, methoxycarbonyl and ethoxycarbonyl. The group may be a terminal group or a bridging group. If the group is a terminal group it is bonded to the remainder of the molecule through the carbonyl carbon.

“Alkyloxycycloalkyl” refers to an alkyloxy-cycloalkyl-group in which the alkyloxy and cycloalkyl moieties are as defined herein. The group may be a terminal group or a bridging group. If the group is a terminal group it is bonded to the remainder of the molecule through the cycloalkyl group.

“Alkyloxyheteroaryl” refers to an alkyloxy-heteroaryl-group in which the alkyloxy and heteroaryl moieties are as defined herein. The group may be a terminal group or a bridging group. If the group is a terminal group it is bonded to the remainder of the molecule through the heteroaryl group.

“Alkyloxyheterocycloalkyl” refers to an alkyloxy-heterocycloalkyl-group in which the alkyloxy and heterocycloalkyl moieties are as defined herein. The group may be a terminal group or a bridging group. If the group is a terminal group it is bonded to the remainder of the molecule through the heterocycloalkyl group.

“Alkylsulfinyl” means an alkyl-S—(═O)— group in which alkyl is as defined herein. The alkyl group is preferably a C₁-C₆ alkyl group. Exemplary alkylsulfinyl groups include, but not limited to, methylsulfinyl and ethylsulfinyl. The group may be a terminal group or a bridging group. If the group is a terminal group it is bonded to the remainder of the molecule through the sulfur atom.

“Alkylsulfonyl” refers to an alkyl-S(═O)₂— group in which alkyl is as defined above. The alkyl group is preferably a C₁-C₆ alkyl group. Examples include, but not limited to methylsulfonyl and ethylsulfonyl. The group may be a terminal group or a bridging group. If the group is a terminal group it is bonded to the remainder of the molecule through the sulfur atom.

“Alkynyl” as a group or part of a group means an aliphatic hydrocarbon group containing a carbon-carbon triple bond and which may be straight or branched preferably having from 2-12 carbon atoms, more preferably 2-10 carbon atoms, more preferably 2-6 carbon atoms in the normal chain. Exemplary structures include, but are not limited to, ethynyl and propynyl. The group may be a terminal group or a bridging group.

“Alkynyloxy” refers to an alkynyl-O— group in which alkynyl is as defined herein. Preferred alkynyloxy groups are C₁-C₆ alkynyloxy groups. The group may be a terminal group or a bridging group. If the group is a terminal group it is bonded to the remainder of the molecule through the oxygen atom.

“Amino acid” as a group or part of a group means having at least one primary, secondary, tertiary or quaternary amino group, and at least one acid group, wherein the acid group may be a carboxylic, sulfonic, or phosphonic acid, or mixtures thereof The amino groups may be “alpha”, “beta”, “gamma” . . . to “omega” with respect to the acid group(s). The amino acid may be natural or synthetic, and may include their derivatives. The backbone of the “amino acid” may be substituted with one or more groups selected from halogen, hydroxy, guanido, heterocyclic groups. Thus the term “amino acids” also includes within its scope glycine, alanine, valine, leucine, isoleucine, methionine, proline, phenylalanine, tryptophan, serine, threonine, cysteine, tyrosine, asparagine, glutamine, asparte, glutamine, lysine, arginine and histidine, taurine, betaine, N-methylalanine etc. (L) and (D) forms of amino acids are included in the scope of this disclosure. Additionally, the amino acids suitable for use in the present disclosure may be derivatized to include amino acids that are hydroxylated, phosphorylated, sulfonated, acylated, and glycosylated, to name a few.

“Amino acid residue” refers to amino acid structures that lack a hydrogen atom of the amino group (—NH—CHR—COOH), or the hydroxy moiety of the carboxygroup (NH2-CHR—CO—), or both (—NH—CHR—CO—).

“Amino” refers to groups of the form —NRₐR_{b} wherein Rₐ and R_{b} are individually selected from the group including but not limited to hydrogen, optionally substituted alkyl, optionally substituted alkenyl, optionally substituted alkynyl, and optionally substituted aryl groups.

The terms “aminocarbonyl” group and “carbonylamino” group can be used interchangeably and are used to describe a —CO—NR₂ group.

“Aminoalkyl” means an NH₂-alkyl-group in which the alkyl group is as defined herein. The group may be a terminal group or a bridging group. If the group is a terminal group it is bonded to the remainder of the molecule through the alkyl group.

“Aminosulfonyl” means an NH₂—S(═O)₂— group. The group may be a terminal group or a bridging group. If the group is a terminal group it is bonded to the remainder of the molecule through the sulfur atom.

“Aryl” as a group or part of a group denotes (i) an optionally substituted monocyclic, or fused polycyclic, aromatic carbocycle (ring structure having ring atoms that are all carbon) preferably having from 6 to 12 atoms per ring. Examples of aryl groups include phenyl, naphthyl, and the like; (ii) an optionally substituted partially saturated bicyclic aromatic carbocyclic moiety in which a phenyl and a C₅₋₇ cycloalkyl or C₅₋₇ cycloalkenyl group are fused together to form a cyclic structure, such as tetrahydronaphthyl, indenyl or indanyl. The group may be a terminal group or a bridging group. Typically an aryl group is a C₆-C₁₈ aryl group.

“Arylalkenyl” means an aryl-alkenyl-group in which the aryl and alkenyl are as defined herein. Exemplary arylalkenyl groups include phenylallyl. The group may be a terminal group or a bridging group. If the group is a terminal group it is bonded to the remainder of the molecule through the alkenyl group.

“Arylalkyl” means an aryl-alkyl-group in which the aryl and alkyl moieties are as defined herein. Preferred arylalkyl groups contain a C₁₋₅ alkyl moiety. Exemplary arylalkyl groups include benzyl, phenethyl, 1-naphthalenemethyl and 2-naphthalenemethyl. The group may be a terminal group or a bridging group. If the group is a terminal group it is bonded to the remainder of the molecule through the alkyl group.

“Arylalkyloxy” refers to an aryl-alkyl-O— group in which the alkyl and aryl are as defined herein. The group may be a terminal group or a bridging group. If the group is a terminal group it is bonded to the remainder of the molecule through the oxygen atom.

“Arylamino” includes both mono-arylamino and di-arylamino unless specified. Mono-arylamino means a group of formula arylNH-, in which aryl is as defined herein. Di-arylamino means a group of formula (aryl)₂N— where each aryl may be the same or different and are each as defined herein for aryl. The group may be a terminal group or a bridging group. If the group is a terminal group it is bonded to the remainder of the molecule through the nitrogen atom.

“Arylheteroalkyl” means an aryl-heteroalkyl-group in which the aryl and heteroalkyl moieties are as defined herein. The group may be a terminal group or a bridging group. If the group is a terminal group it is bonded to the remainder of the molecule through the heteroalkyl group.

“Aryloxy” refers to an aryl-O— group in which the aryl is as defined herein. Preferably the aryloxy is a C₆-C₁₈ aryloxy, more preferably a C₆-C₁₀ aryloxy. The group may be a terminal group or a bridging group. If the group is a terminal group it is bonded to the remainder of the molecule through the oxygen atom.

“Arylsulfonyl” means an aryl-S(═O)₂— group in which the aryl group is as defined herein. The group may be a terminal group or a bridging group. If the group is a terminal group it is bonded to the remainder of the molecule through the sulfur atom.

A “bond” is a linkage between atoms in a compound or molecule. The bond may be a single bond, a double bond, or a triple bond, as valency permits.

“Cycloaliphatic” means non-aromatic, cyclic organic compounds.

“Cycloalkenyl” means a non-aromatic monocyclic or multicyclic ring system containing at least one carbon-carbon double bond and preferably having from 5-10 carbon atoms per ring. Exemplary monocyclic cycloalkenyl rings include cyclopentenyl, cyclohexenyl or cycloheptenyl. The cycloalkenyl group may be substituted by one or more substituent groups. A cycloalkenyl group typically is a C₃-C₁₂ alkenyl group. The group may be a terminal group or a bridging group.

“Cycloalkyl” refers to a saturated monocyclic or fused or bridged or spiro polycyclic, carbocycle preferably containing from 3 to 9 carbon atoms per ring, such as cyclopropyl, cyclobutyl, cyclopentyl, cyclohexyl and the like, unless otherwise specified. It includes monocyclic systems such as cyclopropyl and cyclohexyl, bicyclic systems such as decalin, and polycyclic systems such as adamantane. A cycloalkyl group typically is a C₃-C₁₂ alkyl group. The group may be a terminal group or a bridging group.

“Cycloalkylalkyl” means a cycloalkyl-alkyl-group in which the cycloalkyl and alkyl moieties are as defined herein. Exemplary monocycloalkylalkyl groups include cyclopropylmethyl, cyclopentylmethyl, cyclohexylmethyl and cycloheptylmethyl. The group may be a terminal group or a bridging group. If the group is a terminal group it is bonded to the remainder of the molecule through the alkyl group.

“Cycloalkylalkenyl” means a cycloalkyl-alkenyl-group in which the cycloalkyl and alkenyl moieties are as defined herein. The group may be a terminal group or a bridging group. If the group is a terminal group it is bonded to the remainder of the molecule through the alkenyl group.

“Cycloalkylheteroalkyl” means a cycloalkyl-heteroalkyl-group in which the cycloalkyl and heteroalkyl moieties are as defined herein. The group may be a terminal group or a bridging group. If the group is a terminal group it is bonded to the remainder of the molecule through the heteroalkyl group.

“Cycloalkyloxy” refers to a cycloalkyl-O— group in which cycloalkyl is as defined herein. Preferably the cycloalkyloxy is a C₁-C₆ cycloalkyloxy. Examples include, but are not limited to, cyclopropanoxy and cyclobutanoxy. The group may be a terminal group or a bridging group. If the group is a terminal group it is bonded to the remainder of the molecule through the oxygen atom.

“Cycloalkenyloxy” refers to a cycloalkenyl-O— group in which the cycloalkenyl is as defined herein. Preferably the cycloalkenyloxy is a C₁-C₆ cycloalkenyloxy. The group may be a terminal group or a bridging group. If the group is a terminal group it is bonded to the remainder of the molecule through the oxygen atom.

“Cycloamino” refers to a saturated monocyclic, bicyclic, or polycyclic ring containing at least one nitrogen atom in at least one ring. Each ring is preferably containing from 3 to 10 carbon atoms per ring, more preferably 4 to 7 carbon atoms per ring. The group may be a terminal group or a bridging group. If the group is a terminal group it is bonded to the remainder of the molecule through the nitrogen atom.

“Haloalkyl” refers to an alkyl group as defined herein in which one or more of the hydrogen atoms has been replaced with a halogen atom selected from the group consisting of fluorine, chlorine, bromine and iodine. A haloalkyl group typically has the formula CₙH₍₂ₙ₊₁₋ₘ₎Xₘ wherein each X is independently selected from the group consisting of F, Cl, Br and I . In groups of this type n is typically from 1 to 10, more preferably from 1 to 6, most preferably 1 to 3. m is typically 1 to 6, more preferably 1 to 3. Examples of haloalkyl include fluoromethyl, difluoromethyl and trifluoromethyl.

“Haloalkenyl” refers to an alkenyl group as defined herein in which one or more of the hydrogen atoms has been replaced with a halogen atom independently selected from the group consisting of F, Cl, Br and I.

“Haloalkynyl” refers to an alkynyl group as defined herein in which one or more of the hydrogen atoms has been replaced with a halogen atom independently selected from the group consisting of F, Cl, Br and I.

“Halogen” represents chlorine, fluorine, bromine or iodine.

“Heteroalkyl” refers to a straight-or branched-chain alkyl group preferably having from 2 to 12 carbon atoms, more preferably 2 to 6 carbon atoms in the chain, one or more of which has been replaced by a heteroatom selected from S, O, P and N. Exemplary heteroalkyls include alkyl ethers, secondary and tertiary alkyl amines, amides, alkyl sulfides, and the like. Examples of heteroalkyl also include hydroxyC₁-C₆alkyl, C₁-C₆alkyloxyC₁-C₆alkyl, aminoC₁-C₆alkyl, C₁-C₆alkylaminoC₁-C₆alkyl, and di(C₁-C₆alkyl)aminoC₁-C₆alkyl. The group may be a terminal group or a bridging group.

“Heteroalkyloxy” refers to a heteroalkyl-O— group in which heteroalkyl is as defined herein. Preferably the heteroalkyloxy is a C₁-C₆ heteroalkyloxy. The group may be a terminal group or a bridging group.

“Heteroaryl” either alone or part of a group refers to groups containing an aromatic ring (preferably a 5 or 6 membered aromatic ring) having one or more heteroatoms as ring atoms in the aromatic ring with the remainder of the ring atoms being carbon atoms. Suitable heteroatoms include nitrogen, oxygen and sulphur. Examples of heteroaryl include thiophene, benzothiophene, benzofuran, benzimidazole, benzoxazole, benzothiazole, benzisothiazole, naphtho[2,3-b]thiophene, furan, isoindolizine, xantholene, phenoxatine, pyrrole, imidazole, pyrazole, pyridine, pyrazine, pyrimidine, pyridazine, tetrazole, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, phenoxazine, 2-, 3- or 4-pyridinyl, 2-, 3-, 4-, 5-, or 8-quinolinyl, 1-, 3-, 4-, or 5-isoquinolinyl 1-, 2-, or 3-indolyl, and 2-, or 3-thiophenyl. A heteroaryl group is typically a C₁-C₁₈ heteroaryl group. A heteroaryl group may comprise 3 to 8 ring atoms. A heteroaryl group may comprise 1 to 3 heteroatoms independently selected from the group consisting of N, O and S. The group may be a terminal group or a bridging group.

“Heteroarylalkyl” means a heteroaryl-alkyl group in which the heteroaryl and alkyl moieties are as defined herein. Preferred heteroarylalkyl groups contain a lower alkyl moiety. Exemplary heteroarylalkyl groups include pyridinylmethyl. The group may be a terminal group or a bridging group. If the group is a terminal group it is bonded to the remainder of the molecule through the alkyl group.

“Heteroarylalkenyl” means a heteroaryl-alkenyl-group in which the heteroaryl and alkenyl moieties are as defined herein. The group may be a terminal group or a bridging group. If the group is a terminal group it is bonded to the remainder of the molecule through the alkenyl group.

“Heteroarylheteroalkyl” means a heteroaryl-heteroalkyl-group in which the heteroaryl and heteroalkyl moieties are as defined herein. The group may be a terminal group or a bridging group. If the group is a terminal group it is bonded to the remainder of the molecule through the heteroalkyl group.

“Heteroarylamino” refers to groups containing an aromatic ring (preferably 5 or 6 membered aromatic ring) having at least one nitrogen and at least another heteroatom as ring atoms in the aromatic ring, preferably from 1 to 3 heteroatoms in at least one ring. Suitable heteroatoms include nitrogen, oxygen and sulphur. Arylamino and aryl is as defined herein. The group may be a terminal group or a bridging group. If the group is a terminal group it is bonded to the remainder of the molecule through the nitrogen atom.

“Heteroaryloxy” refers to a heteroaryl-O— group in which the heteroaryl is as defined herein. Preferably the heteroaryloxy is a C₁-C₁₈heteroaryloxy. The group may be a terminal group or a bridging group. If the group is a terminal group it is bonded to the remainder of the molecule through the oxygen atom.

“Heterocyclic” refers to saturated, partially unsaturated or fully unsaturated monocyclic, bicyclic or polycyclic ring system containing at least one heteroatom selected from the group consisting of nitrogen, sulfur and oxygen as a ring atom. Examples of heterocyclic moieties include heterocycloalkyl, heterocycloalkenyl and heteroaryl.

“Heterocycloalkenyl” refers to a heterocycloalkyl as defined herein but containing at least one double bond. A heterocycloalkenyl group typically is a C₂-C₁₂ heterocycloalkenyl group. The group may be a terminal group or a bridging group.

“Heterocycloalkyl” refers to a saturated monocyclic, fused or bridged or spiro polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidinyl, tetrahydrofuranyl, tetrahydrothiofuranyl, piperidinyl, piperazinyl, tetrahydropyranyl, morpholino, 1 ,3-diazapane, 1 ,4-diazapane, 1,4-oxazepane, and 1,4-oxathiapane. A heterocycloalkyl group typically is a C₂-C₁₂ heterocycloalkyl group. A heterocycloalkyl group may comprise 3 to 9 ring atoms. A heterocycloalkyl group may comprise 1 to 3 heteroatoms independently selected from the group consisting of N, O and S. The group may be a terminal group or a bridging group.

“Heterocycloalkylalkyl” refers to a heterocycloalkyl-alkyl-group in which the heterocycloalkyl and alkyl moieties are as defined herein. Exemplary heterocycloalkylalkyl groups include (2-tetrahydrofuranyl)methyl, (2-tetrahydrothiofuranyl)methyl. The group may be a terminal group or a bridging group. If the group is a terminal group it is bonded to the remainder of the molecule through the alkyl group.

“Heterocycloalkylalkenyl” refers to a heterocycloalkyl-alkenyl-group in which the heterocycloalkyl and alkenyl moieties are as defined herein. The group may be a terminal group or a bridging group. If the group is a terminal group it is bonded to the remainder of the molecule through the alkenyl group.

“Heterocycloalkylheteroalkyl” means a heterocycloalkyl-heteroalkyl-group in which the heterocycloalkyl and heteroalkyl moieties are as defined herein. The group may be a terminal group or a bridging group. If the group is a terminal group it is bonded to the remainder of the molecule through the heteroalkyl group.

“Heterocycloalkyloxy” refers to a heterocycloalkyl-O— group in which the heterocycloalkyl is as defined herein. Preferably the heterocycloalkyloxy is a C₁-C₆heterocycloalkyloxy. The group may be a terminal group or a bridging group. If the group is a terminal group it is bonded to the remainder of the molecule through the oxygen atom.

“Heterocycloalkenyloxy” refers to a heterocycloalkenyl-O— group in which heterocycloalkenyl is as defined herein. Preferably the heterocycloalkenyloxy is a C₁-C₆ heterocycloalkenyloxy. The group may be a terminal group or a bridging group. If the group is a terminal group it is bonded to the remainder of the molecule through the oxygen atom.

“Heterocycloamino” refers to a saturated monocyclic, bicyclic, or polycyclic ring containing at least one nitrogen atom and at least another heteroatom selected from nitrogen, sulfur, oxygen, preferably from 1 to 3 heteroatoms in at least one ring. Each ring is preferably from 3 to 10 membered, more preferably 4 to 7 membered. The group may be a terminal group or a bridging group. If the group is a terminal group it is bonded to the remainder of the molecule through the nitrogen atom.

“Hydroxyalkyl” refers to an alkyl group as defined herein in which one or more of the hydrogen atoms have been replaced with an OH group. A hydroxyalkyl group typically has the formula CₙH₍₂ₙ₊₁₋ₓ₎(OH)ₓ. In groups of this type, n is typically from 1 to 10, more preferably from 1 to 6, most preferably from 1 to 3. x is typically from 1 to 6, more preferably from 1 to 4.

“Lower alkyl” as a group means unless otherwise specified, an aliphatic hydrocarbon group which may be straight or branched having 1 to 6 carbon atoms in the chain, more preferably 1 to 4 carbon atoms such as methyl, ethyl, propyl (n-propyl or isopropyl) or butyl (n-butyl, isobutyl or t-butyl). The group may be a terminal group or a bridging group.

“Patient,” as used herein, refers to an animal, preferably a mammal, and most preferably a human.

“Subject” refers to a human or an animal.

“Sulfinyl” means an R—S(═O)— group in which the R group may be OH, alkyl, cycloalkyl, heterocycloalkyl; aryl or heteroaryl group as defined herein. The group may be a terminal group or a bridging group. If the group is a terminal group it is bonded to the remainder of the molecule through the sulfur atom.

“Sulfinylamino” means an R—S(═O)—NH— group in which the R group may be OH, alkyl, cycloalkyl, heterocycloalkyl; aryl or heteroaryl group as defined herein. The group may be a terminal group or a bridging group. If the group is a terminal group it is bonded to the remainder of the molecule through the nitrogen atom.

“Sulfonyl” means an R—S(═O)₂— group in which the R group may be OH, alkyl, cycloalkyl, heterocycloalkyl; aryl or heteroaryl group as defined herein. The group may be a terminal group or a bridging group. If the group is a terminal group, it is bonded to the remainder of the molecule through the sulfur atom.

“Sulfonylamino” means an R—S(═O)₂—NH— group. The group may be a terminal group or a bridging group. If the group is a terminal group it is bonded to the remainder of the molecule through the nitrogen atom.

It is understood that included in the family of compounds of Formula (I) are isomeric forms including diastereomers, enantiomers, tautomers, and geometrical isomers in “E” or “Z” configurational isomer or a mixture of E and Z isomers. It is also understood that some isomeric forms such as diastereomers, enantiomers, and geometrical isomers can be separated by physical and/or chemical methods and by those skilled in the art.

Some of the compounds of the disclosed embodiments may exist as single stereoisomers, racemates, and/or mixtures of enantiomers and/or diastereomers. All such single stereoisomers, racemates and mixtures thereof, are intended to be within the scope of the subject matter described and claimed.

Additionally, Formula (I) is intended to cover, where applicable, solvated as well as unsolvated forms of the compounds. Thus, each formula includes compounds having the indicated structure, including the hydrated as well as the non-hydrated forms.

Further, it is possible that compounds of the invention may contain more than one asymmetric carbon atom. In those compounds, the use of a solid line to depict bonds to asymmetric carbon atoms is meant to indicate that all possible stereoisomers are meant to be included. The use of a solid line to depict bonds to one or more asymmetric carbon atoms in a compound of the invention and the use of a solid or dotted wedge to depict bonds to other asymmetric carbon atoms in the same compound is meant to indicate that a mixture of diastereomers is present.

The term “optionally substituted” as used herein means the group to which this term refers may be unsubstituted, or may be substituted with one or more groups independently selected from alkyl, alkenyl, alkynyl, thioalkyl, cycloalkyl, aminocycloalkyl, cycloalkylalkyl, cycloalkenyl, cycloalkylalkenyl, heterocycloalkyl, cycloalkylheteroalkyl, cycloalkyloxy, cycloalkylaminocarbonyl, cycloalkenyloxy, cycloamino, halogen, carboxyl, haloalkyl, haloalkenyl, haloalkynyl, alkynyloxy, heteroalkyl, heteroalkyloxy, hydroxyl, hydroxyalkyl, alkoxy, alkyloxyalkyloxyalkyl, cycloalkylalkyloxyalkyl, thioalkoxy, alkenyloxy, haloalkoxy, haloalkenyloxy, nitro, amino, aminocarbonyl, aminocarbonylalkyl, azidoalkyl, nitroalkyl, nitroalkenyl, nitroalkynyl, nitroheterocyclyl, alkylamino, alkylaminocarbonyl, dialkylamino, dialkylaminocarbonyl, alkenylamine, alkylcarbonylamino, aminoalkyl, alkynylamino, acyl, alkyloxy, alkyloxyalkyl, alkyloxyaryl, alkyloxycarbonyl, alkyloxycycloalkyl, alkyloxyheteroaryl, alkyloxyheterocycloalkyl, alkenoyl, alkynoyl, acylamino, diacylamino, acyloxy, alkylsulfonyloxy, heterocyclic, heterocycloalkenyl, heterocycloalkyl, heterocycloalkylalkyl, heterocycloalkylalkenyl, heterocycloalkylheteroalkyl, heterocycloalkyloxy, heterocycloalkenyloxy, heterocycloxy, heterocycloamino, haloheterocycloalkyl, alkylsulfinyl, alkylsulfonyl, alkylsulfenyl, alkylcarbonyloxy, alkylthio, acylthio, aminosulfonyl, phosphorus-containing groups such as phosphono and phosphinyl, sulfinyl, sulfinylamino, sulfonyl, sulfonylamino, alkylsulfamoyl, aryl, heteroaryl, heteroarylalkyl, heteroarylalkenyl, heteroarylheteroalkyl, heteroarylamino, heteroaryloxy, arylalkenyl, arylalkyl, alkylaryl, alkylheteroaryl, aryloxy, arylsulfonyl, cyano, cyanate, isocyanate, —C(O)NH(alkyl), and —C(O)N(alkyl)₂. The number of carbon and hetero atoms in the groups of the optional substituents is as defined for the groups below, e.g. an alkyl or alkylene moiety can be a C₁-C₁₂ alkyl.

Preferably, the halogen is chlorine, fluorine, bromine or iodine, the alkyl is an optionally substituted C₁-C₁₂ alkyl, the alkenyl is an optionally substituted C₁-C₁₂ alkenyl, the alkynyl is a C₁-C₁₂ alkynyl, the thioalkyl is an optionally substituted C₁-C₁₂ thioalkyl comprising 1 or 2 sulfur atoms, the alkyloxy is an optionally substituted C₁-C₆ alkyl-O— group, the cycloalkyl is an optionally substituted C₃-C₉ cycloalkyl, the aminocycloalkyl is an optionally substituted C₃-C₉ aminocycloalkyl, the cycloalkylalkyl is an optionally substituted C₃ to C₉ cycloalkylalkyl, the cycloalkenyl is an optionally substituted C₃-C₉ cycloalkenyl, the cycloalkylalkenyl is an optionally substituted C₃ to C₉ cycloalkylalkenyl, the heterocycloalkyl is an optionally substituted heterocycloalkyl having a ring atom number of 3 to 8 and having 1 to 3 heteroatoms independently selected from the group consisting of N, O and S, the cycloalkylheteroalkyl is an optionally substituted cycloalkylheteroalkyl having a ring atom number of 3 to 8 and having 1 to 3 heteroatoms independently selected from the group consisting of N, O and S, the cycloalkyloxy is an optionally substituted cycloalkyloxy having a ring atom number of 3 to 8 and having 1 or 2 oxygen atoms, the cycloalkylaminocarbonyl is an optionally substituted cycloalkylaminocarbonyl having a ring number of 3 to 8 and having a —CO—NH₂ group, the cycloalkenyloxy is an optionally substituted cycloalkenyloxy having a ring atom number of 3 to 8 and having 1 or 2 oxygen atoms, the cycloamino is an optionally substituted cycloamino having a ring atom number of 3 to 8 and having 1 or 2 nitrogen atoms, halo is selected from the group consisting of fluoro, chloro, bromo and iodo, haloalkyl is an optionally substituted C₁-C₁₂ haloalkyl having at least one halo group selected from the group consisting of fluoro, chloro, bromo and iodo, haloalkenyl is an optionally substituted C₁-C₁₂ haloalkenyl having at least one halo group selected from the group consisting of fluoro, chloro, bromo and iodo, haloalkynyl is an optionally substituted C₁-C₁₂ haloalkynyl having at least one halo group selected from the group consisting of fluoro, chloro, bromo and iodo, alkenyloxy is an optionally substituted C₁-C₆ alkenyloxy having at least one oxygen atom, alkynyloxy is an optionally substituted C₁-C₆ alkynyloxy having at least one oxygen atom, heteroalkyl is an optionally substituted C₂-C₁₂ alkyl having a least one heteroatom selected from the group consisting of N, O, P and S, heteroalkyloxy is an optionally substituted C₂-C₁₂ alkyl having at least one oxygen atom and at least one other heteroatom selected from the group consisting of N, O, P and S, hydroxyalkyl is a substituted alkyl having the formula CₙH₍₂ₙ₊₁₋ₓ₎(OH)ₓ where n is 1 to 10, the thioalkyloxy is an optionally substituted C₁-C₆ alkyl-O— group having at least one sulfur group, the haloalkyloxy is an optionally substituted C₁-C₆ alkyl-O— group having at least one other substituent selected from the group consisting of fluoro, chloro, bromo and iodo, haloalkenyloxy is an optionally substituted C₁-C₆ alkenyloxy having at least one oxygen atom and at least one other substituent selected from the group consisting of fluoro, chloro, bromo and iodo, the aminocarbonyl is a —CO—NH₂ group, the aminocarbonylalkyl is an optionally substituted C₁ to C₁₂ alkyl group having a —CO—NH₂ group, the azidoalkyl is a C₁-C₁₂-alkyl-N₃ group, the nitroalkyl is an optionally substituted C₁-C₁₂ alkyl having at least one nitro group, the nitroalkenyl is an optionally substituted C₁-C₁₂ alkenyl having at least one nitro group, the nitroalkynyl is an optionally substituted C₁-C₁₂ alkynyl having at least one nitro group, the nitroheterocyclyl is an optionally substituted heterocycloalkyl having a ring atom number of 3 to 8 and having 1 to 3 heteroatoms independently selected from the group consisting of N, O and S and having at least one nitro group, the optionally substituted aryl is an optionally substituted C₆-C₁₈ aryl, the heteroaryl is an optionally substituted heteroaryl having a ring atom number of 3 to 8 and having 1 to 3 heteroatoms independently selected from the group consisting of N, O and S, alkylamino is an optionally substituted alkyl-NH— group having a C₁-C₆ alkyl group, dialkylamino is an optionally substituted (alkyl)₂N— group having a C₁-C₆ alkyl group, alkylaminocarbonyl is an optionally substituted alkyl-NH—CO— group having a C₁-C₆ alkyl group, dialkylaminocarbonyl is an optionally substituted (alkyl)₂N—CO— group having a C₁-C₆ alkyl group alkenylamine is an optionally substituted alkenyl-NH— group having a C₁-C₆ alkenyl group, alkylcarbonylamino is an optionally substaited Cᵢ-C₁₂-alkyl-CO-NH₂ group, alkynyl amino is an optionally substituted alkynyl-NH— group having a C₁-C₆ alkynyl group, alkyloxyalkyl is an optionally substituted alkyloxy group having a C₁-C₆ alkyl group, the alkyloxyalkyloxyalkyl is an optionally substituted C₁ to C₆ alkyloxyalkyl substituted with a C₁ to C₆ alkyloxyalkyl, cycloalkylalkyloxyalkyl is a C₁ to C₆ alkyloxyalkyl substaituted with a C₃ to C₈ cycloalkyl group, alkyloxyaryl is an optionally substituted alkyloxy group having an optionally substituted C₆-C₁₈ aryl, alkyloxycarbonyl is a an optionally substituted C₁-C₁₆ alkyloxy having a carbonyl group, alkyloxycyclocarbonyl is an optionally substituted optionally substituted C₃ to C₉ cycloalkylalkyl having a carbonyl group and an alkoxy group, the alkyloxyheteroaryl is an optionally substituted heteroaryl having a ring atom number of 3 to 8 and having 1 to 3 heteroatoms independently selected from the group consisting of N, O and S and having a C₁-C₆ alkyloxy group, alkyloxyheterocycloalkyl is an optionally substituted an optionally substituted heterocycloalkyl having a ring atom number of 3 to 8 and having 1 to 3 heteroatoms independently selected from the group consisting of N, O and S having a C₁-C₆ alkyloxy group, alkanoyl is an optionally substituted C₁-C₁₂ alkyl having a carbonyl group, alkenoyl is an optionally substituted C₁-C₁₂ alkenyl having a carbonyl group, alkynoyl is an optionally substituted C₁-C₁₂ alkynyl having a carbonyl group, acylamino is an optionally substituted R—C(═O)—NH— group in which the R group may be a C₁-C₁₂ alkyl, C₃-C₁₂ cycloalkyl, C₃-C₁₂ heterocycloalkyl having 1 to 3 heteroatoms independently selected from the group consisting of N, O and S, aryl having a ring atom number of 3 to 8 or heteroaryl group having a ring atom number of 3 to 8 and having 1 to 3 heteroatoms independently selected from the group consisting of N, O and S, diacylamino is an optionally substituted [R—C(═O)]₂—NH group in which the R group may be a C₁-C₁₂ alkyl, C₃-C₁₂ cycloalkyl, C₃-C₁₂ heterocycloalkyl having 1 to 3 heteroatoms independently selected from the group consisting of N, O and S aryl having a ring atom number of 3 to 8 or heteroaryl group having a ring atom number of 3 to 8 and having 1 to 3 heteroatoms independently selected from the group consisting of N, O and S, the acyloxy is a C₁-C₁₂ acyloxy, the alkylsufonyloxy is an optionally substituted C₁-C₆ alkyl-O— group having at least one sulfonyl group, the alkylsulfamoyl is an optionally substituted C₁-C₆ alkyl-O group having at last one sulfamoyl group, the heterocycloalkenyl is a heterocycloalkenyl having a ring atom number of 3 to 8 and having 1 to 3 heteroatoms independently selected from the group consisting of N, O and S, the heterocycloalkyl is a heterocycloalkyl having a ring atom number of 3 to 8 and having 1 to 3 heteroatoms independently selected from the group consisting of N, O and S, the heterocycloalkylalkyl is an optionally substituted C₃ to C₉ cycloalkylalkyl having 1 to 3 heteroatoms independently selected from the group consisting of N, O and S, the heterocycloalkylalkenyl is an optionally substituted C₃ to C₉ cycloalkylalkenyl having 1 to 3 heteroatoms independently selected from the group consisting of N, O and S, the heterocycloalkylheteroalkyl is an optionally substituted C₃ to C₉ cycloalkylalkenyl having 1 to 3 heteroatoms independently selected from the group consisting of N, O and S, the heterocycloalkyloxy is an optionally substituted C₃ to C₉ cycloalkyl having 1 to 3 heteroatoms independently selected from the group consisting of N, O and S and an optionally substituted C₁-C₆ alkyl-O— group, the heterocycloalkenyloxy is an optionally substituted C₃ to C₉ cycloalknyl having 1 to 3 heteroatoms independently selected from the group consisting of N, O and S and an optionally substituted C₁-C₆ alkyl-O— group, the heterocycloxy is an optionally substituted heterocycloalkyl having a ring atom number of 3 to 8 and having 1 to 3 heteroatoms independently selected from the group consisting of N, O and S and a hydroxyl group, the heterocycloamino is an optionally substituted heterocycloalkyl having a ring atom number of 3 to 8 and having 1 to 3 heteroatoms independently selected from the group consisting of N, O and S and an amino group, the haloheterocycloalkyl is an optionally substituted heterocycloalkyl having a ring atom number of 3 to 8 and having 1 to 3 heteroatoms independently selected from the group consisting of N, O and S and a halo group selected from the group consisting of fluoro, chloro, iodo and bromo, the alkylsulfinyl is an optionally substituted C₁-C₁₂ alkyl group having at least one sulfinyl group, the alkylsulfonyl is an optionally substituted C₁-C₁₂ alkyl group having at least one sulfonyl group, the alkylsulfenyl is an optionally substituted C₁-C₁₂ alkyl group having at least one sulfenyl group, the alkylcarbonyloxy is an optionally substituted C₁-C₁₂ alkyl group having at least one carbonyl group and at least one hydroxy group, the alkylthio is an optionally substituted C₁-C₁₂ alkyl group having at least one thiol group, the acylthio is R—C(═O)—S in which R group may be a C₁-C₁₂ alkyl, C₃-C₁₂ cycloalkyl, C₃-C₁₂ heterocycloalkyl having 1 to 3 heteroatoms independently selected from the group consisting of N, O and S, aryl having a ring atom number of 3 to 8 or heteroaryl group having a ring atom number of 3 to 8 and having 1 to 3 heteroatoms independently selected from the group consisting of N, O and S, the heteroarylalkyl or alkylheteroaryl is an optionally substituted heteroaryl having a ring atom number of 3 to 8 and having 1 to 3 heteroatoms independently selected from the group consisting of N, O and S and a C₁-C₁₂ alkyl, the heteroarylalkenyl is an optionally substituted heteroaryl having a ring atom number of 3 to 8 and having 1 to 3 heteroatoms independently selected from the group consisting of N, O and S and a C₁-C₁₂ alkenyl, the heteroarylheteroalkyl is an optionally substituted heteroaryl having a ring atom number of 3 to 8 and having 1 to 3 heteroatoms independently selected from the group consisting of N, O and S and a C₁-C₁₂ alkyl having at least one heteroatom selected from the group consisting of N, O and S, the heteroarylamino is an optionally substituted heteroaryl having a ring atom number of 3 to 8 and having 1 to 3 heteroatoms independently selected from the group consisting of N, O and S and an amino group, the heteroaryloxy is an optionally substituted heteroaryl having a ring atom number of 3 to 8 and having at least one oxygen group, the arylalkenyl is an optionally substituted heteroaryl having a ring atom number of 3 to 8 and having 1 to 3 heteroatoms independently selected from the group consisting of N, O and S and a C₁-C₁₂ alkenyl, the arylalkyl or alkylaryl is an optionally substituted heteroaryl having a ring atom number of 3 to 8 and having 1 to 3 heteroatoms independently selected from the group consisting of N, O and S and a C₁-C₁₂ alkyl, the aryloxy is an optionally substituted heteroaryl having a ring atom number of 3 to 8 and having at least one oxygen atom, or the arylsulfonyl is an optionally substituted heteroaryl having a ring atom number of 3 to 8 and having at least one sulfur atom.

The term “pharmaceutically acceptable salts” refers to salts that retain the desired biological activity of the above-identified compounds, and include pharmaceutically acceptable acid addition salts and base addition salts. Suitable pharmaceutically acceptable acid addition salts of compounds of Formula (I) may be prepared from an inorganic acid or from an organic acid. Examples of such inorganic acids are hydrochloric, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic, arylsulfonic. Additional information on pharmaceutically acceptable salts can be found in Remington's Pharmaceutical Sciences, 19th Edition, Mack Publishing Co., Easton, Pa. 1995. In the case of agents that are solids, it is understood by those skilled in the art that the inventive compounds, agents and salts may exist in different crystalline or polymorphic forms, all of which are intended to be within the scope of the present disclosure and specified formulae.

“Prodrug” means a compound that undergoes conversion to a compound of formula (I) within a biological system, usually by metabolic means (e.g. by hydrolysis, reduction or oxidation). For example an ester prodrug of a compound of formula (I) containing a hydroxyl group may be converted by hydrolysis in vivo to the parent molecule. Suitable esters of compounds of formula (I) containing a hydroxyl group, are for example formates, acetates, citrates, lactates, tartrates, malonates, oxalates, salicylates, propionates, succinates, fumarates, maleates, methylene-bis-β-hydroxynaphthoates, gentisates, isethionates, di-p-toluoyltartrates, methanesulfonates, ethanesulfonates , benzenesulfonates , p-toluenesulfonates, cyclohexylsulfamates, and quinates. As another example an ester prodrug of a compound of formula (I) containing a carboxy group may be convertible by hydrolysis in vivo to the parent molecule. (Examples of ester prodrugs are those described by F. J. Leinweber, Drug Metab. Res., 18:379, 1987). Similarly, an acyl prodrug of a compound of formula (I) containing an amino group may be converted by hydrolysis in vivo to the parent molecule (Many examples of prodrugs for these and other functional groups, including amines, are described in Prodrugs: Challenges and Rewards (Parts 1 and 2); Ed V. Stella, R. Borchardt, M. Hageman, R. Oliyai, H. Maag and J Tilley; Springer, 2007)

The term “therapeutically effective amount” or “effective amount” is an amount sufficient to effect beneficial or desired clinical results. An effective amount can be administered in one or more administrations. An effective amount is typically sufficient to palliate, ameliorate, stabilize, reverse, slow or delay the progression of the disease state.

The term “functional equivalent” is intended to include variants of the specific protein lysine methyl transferase species described herein. It will be understood that the protein lysine methyl transferases may have isoforms, such that while the primary, secondary, tertiary or quaternary structure of a given protein lysine methyl transferase isoform is different to the prototypical protein lysine methyl transferase, the molecule maintains biological activity as a protein lysine methyl transferase. Isoforms may arise from normal allelic variation within a population and include mutations such as amino acid substitution, deletion, addition, truncation, or duplication. Also included within the term “functional equivalent” are variants generated at the level of transcription. Enzymes have isoforms that arise from transcript variation. Other functional equivalents include protein lysine methyl transferases having altered post-translational modification such as glycosylation.

The term “reprogramming cells” is intended to include erasure and remodeling of epigenetic marks, such as DNA methylation, during mammalian development.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means ±10% of the stated value, more typically ±7.5% of the stated value, more typically ±5% of the stated value, more typically ±4% of the stated value, more typically ±3% of the stated value, more typically, ±2% of the stated value, even more typically ±1% of the stated value, and even more typically ±0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 shows the effect of compounds on the methyltransferase activity of SMYD3 using MAP3K2 peptide as a substrate and refers to dose-response curves showing the effect of compounds on the methyltransferase activity of SMYD3 using MAP3K2 peptide as a substrate; for compound A066 (FIG. 1A), for compound A088 (FIG. 1B); for compound B019 (FIG. 1C); and for compound A074 (FIG. 1D).

FIG. 2 refers to dose-response curves showing that SMYD3 compounds inhibit the proliferation of HCC, Colorectal, Lung and Pancreatic carcinoma cell lines. FIG. 2A is a dose response curve showing inhibition of HepG2, FIG. 2B is a dose response curve showing inhibition of HCT116, FIG. 2C is the dose response curve showing inhibition of A549, FIG. 2D is the dose response curve showing inhibition of CFPAC-1 and FIG. 2E is a dose response curve showing inhibition of HPAF-II.

FIG. 3 refers to an image of aWestern blot showing SMYD3 target engagement and inhibition of MAP3K2 methylation following treatment with 25.0 μM of compound B019 and X4, in HEK293 cells transiently transfected with Myc-SMYD3.

FIG. 4 refers to colony images in 24-well plate and the corresponding dose response curves of 2 sets of experiments using HepG2 cells. FIG. 4A is a colony image and FIG. 4B is a dose response curve with compound A074. FIG. 4C is a colony image and FIG. 4D is a dose response curve with compound B019.

### DETAILED DESCRIPTION OF EMBODIMENTS

The present disclosure provides a compound of the following Formula (I);

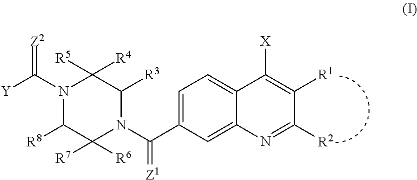

Z¹ and Z² may be selected from O, S or NH. Z¹ may be O. Z² may be O.

X may be a halogen. X may be chloro.

R¹ and R² may be independently selected from the group consisting of a bond, H, halogen, cyano, optionally substituted alkyl, optionally substituted alkene, optionally substituted alkyne, optionally substituted alkoxy, optionally substituted amino, optionally substituted cycloalkyl, optionally substituted heterocycloalkyl, optionally substituted aryl and optionally substituted heteroaryl.

R¹ and R² may optionally be taken together to form an optionally substituted alkylene bridge or an optionally substituted alkylene bridge wherein one or two alkylene units may be replaced with O, NH or S.

R¹ and R² may optionally form an optionally substituted aryl or optionally substituted heteroaryl together with the ring atoms that they are bond to.

R³, R⁴, R⁵, R⁶, R⁷ and R⁸ may be independently absent, or selected from the group consisting of a bond, H, halogen, optionally substituted alkyl, optionally substituted alkene, optionally substituted alkyne, optionally substituted alkoxy, optionally substituted amino, optionally substituted acyl, optionally substituted cycloalkyl, optionally substituted heterocycloalkyl, optionally substituted aryl and optionally substituted heteroaryl.

R³, R⁴, R⁵, R⁶, R⁷ and R⁸ may be independently hydrogen or methyl.

R³, R⁴, R⁵, R⁶, R⁷ or R⁸ may be taken together to form an optionally substituted cycloalkyl, or optionally substituted alkylene bridge or an optionally substituted alkylene bridge wherein one or two alkylene units may be replaced with O, NH or S.

Y may be selected from the group consisting of R⁹, OR⁹ or NHR⁹, wherein R⁹ is an optionally substituted C₃ to C₁₀ alkyl, optionally substituted C₃ to C₁₀ alkenyl, optionally substituted C₃ to C₁₀ alkynyl, optionally substituted C₃ to C₇ cycloalkyl, optionally substituted C₂ to C₁₀ haloalkyl, a substituted 5-membered heteroaryl comprising two or three heteroatoms selected from N, O or S or a C₁ to C₂ alkyl substituted with an optionally substituted 5-membered heterocycloalkyl comprising one to two heteroatoms selected from N, O or S.

The compound of Formula (I) may include a pharmaceutically acceptable form or prodrug thereof.

The compound may have the following Formula (II):

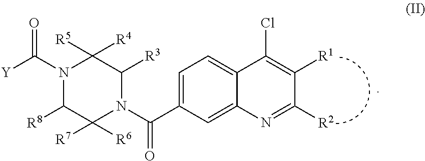

The optionally substituted alkyl may be an optionally substituted C₁-C₁₂ alkyl. The optionally substituted alkyloxy may be an optionally substituted C₁-C₁₆ alkyloxy. The optionally substituted cycloalkyl may be an optionally substituted C₃-C₉ cycloalkyl. The optionally substituted heterocycloalkyl may be an optionally substituted heterocycloalkyl having a ring atom number of 3 to 8 and having 1 to 3 heteroatoms independently selected from the group consisting of N, O and S, the optionally substituted aryl may be an optionally substituted C₆-C₁₈ aryl, the optionally substituted heteroaryl may be an optionally substituted heteroaryl having a ring atom number of 3 to 8 and having 1 to 3 heteroatoms independently selected from the group consisting of N, O and S, the optionally substituted alkenyl may be an optionally substituted C₂-C₁₂ alkenyl or the optionally substituted alkynyl may be an optionally substituted C₂-C₁₂ alkynyl.

R⁹ may be a C₃ to C₁₀ alkyl, optionally substituted C₃ to C₆ alkenyl, optionally substituted C₂ to C₁₀ haloalkyl, or in each case C₃ to C₉ alkyl or C₃ to C₇ cycloalkyl, or substituted oxazolyl, isoxazolyl, 1,2-azole, pyrazolyl, triazolyl, or methylpyrrolidinonyl.

R⁹ may be selected from the group consisting of propyl, butyl, pentyl, —CH₂CH(CH₃)₂, —CH₂CH═CH, 2-fluoroethyl, 3-fluoropropyl, 5-cyclopropylisoxazol-3-yl, 5-isobutylisoxazol-3-yl, 5-methylisoxazol-3-yl 5-methylpyrazol-3-yl, 1-methyl-1,2,3-triazol-4-yl, 1-cycloproyl-1,2,3-triazol-4-yl, 1-tert-butyl-1,2,3-triazol-4-yl, 1-cyclopropyl-1,2-pyrazol-4-yl and (R)-pyrrolidin-2-onyl-5-methyl.

The compound may have the following formula (IIa):

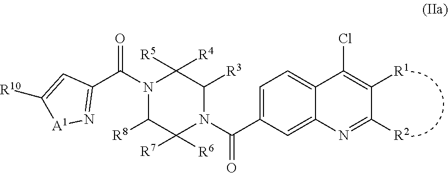

A¹ may be O or NH.

R¹⁰ may be a C₁ to C₉ alkyl or a C₃ to C₇ cycloalkyl. R¹⁰ may be selected from the group consisting of methyl, isobutyl and cyclopropyl.

R¹ and R² may be independently selected from the group consisting of a bond, H, halogen, cyano, optionally substituted alkyl, optionally substituted phenyl, optionally substituted pyrazolyl, optionally substituted thiazolyl, optionally substituted thiophenyl, optionally substituted benzo[d]imidazolyl, optionally substituted indolyl, optionally substituted isoindoyl, optionally substituted indazolyl, optionally substituted pyrrolyl, optionally substituted pyridinyl, optionally substituted benzyl, optionally substituted benzo[d]dioxolyl, optionally substituted benzotriazolyl, optionally substituted benzoxazolyl, optionally substituted benzofuranyl, optionally substituted pyrazolopyridinyl, optionally substituted pyrrolopyrimidinyl, optionally substituted pyrrolopyridinyl, optionally substituted naphthyridinyl, optionally substituted pyrimidinyl, optionally substituted benzothiazolyl, optionally substituted cyclopropyl, amino group optionally substituted with an optionally substituted phenyl and amino group optionally substituted with an optionally substituted pyridinyl.

The compound may have the following Formula (IIb):

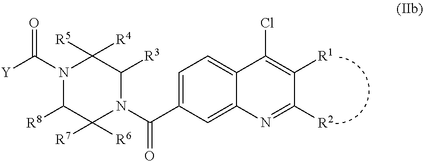

R¹ may be H or halogen. R² may be selected from the group consisting of H, cyano, methyl, ethyl, ethynyl, phenyl, 2-methylphenyl, 3-methylphenyl, 4-methylphenyl, 2-hydroxyphenyl, 3-hydroxyphenyl, 4-hydroxyphenyl, 2-(trifluoromethyl)phenyl, 3-(trifluoromethyl)phenyl, 4-(trifluoromethyl)phenyl, 2-chlorophenyl, 3-chlorophenyl, 4-chlorophenyl, 2-fluorophenyl, 3-fluorophenyl, 4-fluorophenyl, 2-fluoromethylphenyl, 3-fluoromethylphenyl, 4-fluoromethylphenyl, 2-hydroxymethylphenyl, 3-hydroxymethylphenyl, 4-hydroxymethylphenyl, 2-methoxyphenyl, 3-methoxyphenyl, 4-methoxyphenyl, 2-ethoxyethylphenyl, 3-ethoxyethylphenyl, 4-ethoxyethylphenyl, 2-(azidomethyl)phenyl, 3-(azidomethyl)phenyl, 4-(azidomethyl)phenyl, 2,3-difluorophenyl, 2,4-difluorophenyl, 2,5-difluorophenyl, 2,6-difluorophenyl, 3,4-difluorophenyl, 3,5-difluorophenyl, 3,5-difluoro-4-hydroxyphenyl, 3,5-difluoro-4-(aminocarbonyl)phenyl, 3,5-difluoro-4-aminomethylphenyl, 2-cyanophenyl, 3-cyanophenyl, 4-cyanophenyl, 2-(cyanomethyl)phenyl, 3-(cyanomethyl)phenyl, 4-(cyanomethyl)phenyl, 2-nitrophenyl, 3-nitrophenyl, 4-nitrophenyl, 2-aminophenyl, 3-aminophenyl, 4-aminophenyl, 2-(aminomethyl)phenyl, 3-(aminomethyl)phenyl, 4-(aminomethyl)phenyl, 2-(dimethylamino)phenyl, 3-(dimethylamino)phenyl, 4-(dimethylamino)phenyl, 2-(aminocarbonyl)phenyl, 3-(aminocarbonyl)phenyl, 4-(aminocarbonyl)phenyl, 2-(methylaminocarbonyl)phenyl, 3-(methylaminocarbonyl)phenyl, 4-(methylaminocarbonyl)phenyl, 2-(ethylaminocarbonyl)phenyl, 3-(ethylaminocarbonyl)phenyl, 4-(ethylaminocarbonyl)phenyl, 4-(1-ethoxyethyl)phenyl, 4-(2-hydroxy-2-propyl)phenyl, 2-pyridinyl, 3-pyridinyl, 4-pyridinyl, 2-methyl-3-pyridinyl, 4-methyl-3-pyridinyl, 5-methyl-3-pyridinyl, 6-methyl-3-pyridinyl, 6-methoxycarbonyl-3-pyridinyl, thiophenyls such as 2-thiophenyl, 3-thiophenyl, 1-pyrrolidinyl, 2-pyrrolidinyl, 3-pyrrolidinyl, 1-pyrrolyl, 2-pyrrolyl, 3-pyrrolyl, 1-methyl-3-pyrrolyl, 3-(1,2,5-trimethyl)-pyrrolyl, 2-ethynylphenyl, 3-ethynylphenyl, 4-ethynylphenyl, 2-ethylphenyl, 3-ethylphenyl, 4-ethylphenyl, 2-(1-hydroxyethyl)phenyl, 3-(1-hydroxyethyl)phenyl, 4-(1-hydroxyethyl)phenyl, 2-(2-hydroxyethyl)phenyl, 3-(2-hydroxyethyl)phenyl, 4-(2-hydroxyethyl)phenyl, 4-fluoro-3-methylphenyl, 4-fluoro-2-methylphenyl, 3-fluoro-2-methylphenyl, 3-fluoro-4-methylphenyl, 3-fluoro-5-methylphenyl, 2-fluoro-5-methylphenyl, 4-fluoro-3-methoxyphenyl, 4-fluoro-2-methoxyphenyl, 3-fluoro-2-methoxyphenyl, 3-fluoro-4-methoxyphenyl, 3-fluoro-5-methoxyphenyl, 2-fluoro-5-methoxyphenyl, 4-fluoro-3-hydroxyphenyl, 4-fluoro-2-hydroxyphenyl, 4-hydroxy-3-fluorophenyl, 4-hydroxy-2-fluorophenyl, 4-fluoro-3-hydroxymethylphenyl, 4-fluoro-2-hydroxymethylphenyl, 3-fluoro-2-hydroxymethylphenyl, 3-fluoro-4-hydroxymethylphenyl, 3-fluoro-5-hydroxymethylphenyl, 2-fluoro-5-hydroxymethylphenyl, 3-fluoro-4-(2-hydroxy-2-propyl)phenyl, 3-(aminocarbonyl)-4-fluorophenyl, 2-(aminocarbonyl)-4-fluorophenyl, 2-(aminocarbonyl)-3-fluorophenyl, 4-(aminocarbonyl)-3-fluorophenyl, 5-(aminocarbonyl)-3-fluorophenyl, 5-(aminocarbonyl)-2-fluorophenyl, 4-fluoro-3-(methylaminocarbonyl)phenyl, 3-fluoro-4-(methylaminocarbonyl)phenyl, 4-fluoro-2-(methylaminocarbonyl)phenyl, 3-fluoro-2-(methylaminocarbonyl)phenyl, 4-(cyclopropylaminocarbonyl)phenyl, 2-(cyclopropylaminocarbonyl)phenyl, 3-(cyclopropylaminocarbonyl)phenyl, 4-fluoro-3-(cyclopropylaminocarbonyl)phenyl, 3-fluoro-4-(cyclopropylaminocarbonyl)phenyl, 4-fluoro-2-(cyclopropylaminocarbonyl)phenyl, 3-fluoro-2-(cyclopropylaminocarbonyl)phenyl, (3-fluoro-4-(dimethylaminocarbonyl)phenyl, 3-fluoro-5-(dimethylaminocarbonyl)phenyl, 2-fluoro-5-(dimethylaminocarbonyl)phenyl, 4-fluoro-3-(dimethylaminocarbonyl)phenyl, 4-fluoro-2-(dimethylaminocarbonyl)phenyl, 3-fluoro-2-(dimethylaminocarbonyl)phenyl, 3-methyl-4-(methylaminocarbonyl)phenyl, 3-amino-4-fluorophenyl, 2-amino-4-fluorophenyl, 3-aminomethyl-4-fluorophenyl, 2-aminomethyl-4-fluorophenyl, 3-hydroxymethyl-4-methylphenyl, 2-hydroxymethyl-4-methyl-phenyl, 2-hydroxymethyl-3-methyl-phenyl, 4-hydroxymethyl-3-methylphenyl, 5-hydroxymethyl-3-methylphenyl, 5-hydroxymethyl-2-methylphenyl, 2-morpholinophenyl, 3-morpholinophenyl, 4-morpholinophenyl, 2-(pyrrolidin-1-yl)phenyl, 3-(pyrrolidin-1-yl)phenyl, 4-(pyrrolidin-1-yl)phenyl, 4-(1-amino-1-cyclopropyl)phenyl, cyclopropyl, cyclobutyl, cyclopentyl, cyclohexyl, 2-thiazolyl, 4-thiazolyl, 5-thiazolyl, 2-methylthiazolyl, 4-methylthiazolyl, 4-(dimethylamido)phenyl, 2-(dimethylamido)phenyl, 3-(dimethylamido)phenyl, 2-benzylamin, 3-benzylamin, 4-benzylamin, 2-methylaminophenyl, 3-methylaminophenyl, 4-methylaminophenyl, 6-(1-methyl)indazolyl, 6-(2-methyl)indazolyl, 5-(1-methyl)indazolyl, 4-(1-methyl)indazolyl, 3-(1-methyl)indazolyl, 5-(2-methyl)indazolyl, 5-(3-methyl)indazolyl, 5-(1-methyl)pyrazolyl, 4-(1-methyl)-pyrazolyl, 3-(1-methyl) pyrazolyl, 4-(1-isopropyl)-pyrazolyl, 4-(1-difluoromethyl)-pyrazolyl, 4-(5-trifluoromethyl)-pyrazolyl, 4-(1-(2,2,2)-trifluoroethyl)pyrazolyl, 4-(1-cyclopentyl)pyrazolyl, 2-(1-methyl) pyrazolyl-phenyl, 3-(1-methyl) pyrazolyl-phenyl, 4-(imidazol-1-yl)phenyl, 1-imidazolyl, 2-imidazolyl, 3-imidazolyl, 4-(4-methylpiperazino)phenyl, 3-(4-methylpiperazino)phenyl, 2-(4-methylpiperazino)phenyl, 3-[1,2,4]-triazol-4-ylphenyl, 2-[1,2,4]-triazol-4-yl phenyl, 4-[1,2,4]-triazol-4-ylphenyl, 3-(aminomethyl)-4-methoxyphenyl, 3-(aminomethyl)-5-methoxyphenyl, 2-(aminomethyl)-4-methoxyphenyl, 2-(aminomethyl)-5-methoxyphenyl, 2-(aminomethyl)-6-methoxyphenyl, 4-(aminomethyl)-3-methoxyphenyl, 2-(aminomethyl)-3-methoxyphenyl, 4-(dimethylaminomethyl)phenyl, 3-(dimethylaminomethyl)phenyl, 2-(dimethylaminomethyl)phenyl, 4-fluoro-3-(dimethylaminomethyl)phenyl, 4-fluoro-2-(dimethylaminomethyl)phenyl, 4-methoxy-3-methylphenyl, 2-methoxy-4-methylphenyl, 3-methoxy-5-methylphenyl, 3-methoxy-4-methylphenyl, 2-methoxy-5-methylphenyl, 2-methoxy-6-methylphenyl, 2-methoxy-3-methylphenyl, 4-methoxy-3-hydroxymethylphenyl, 3-methoxy-4-hydroxymethylphenyl, 2-methoxy-4-hydroxymethylphenyl, 3-methoxy-5-hydroxymethylphenyl, 2-methoxy-5-hydroxymethylphenyl, 2-methoxy-6-hydroxymethylphenyl, 2-methoxy-3-hydroxymethylphenyl, 4-hydroxy-3-hydroxymethylphenyl, 4-hydroxy-3-methylphenyl, 3-ethoxy-4-hydroxyphenyl, 3-hydroxy-4-methylphenyl, 2-hydroxy-4-methylphenyl, 3-cyano-4-methylphenyl, 4-cyano-3-methylphenyl, 2-cyano-4-methylphenyl, 3-cyano-5-methylphenyl, 2-cyano-5-methylphenyl, 2-cyano-6-methylphenyl, 2-cyano-3-methylphenyl, 4-(aminosulfonyl)phenyl, 3-(aminosulfonyl)phenyl, 2-(aminosulfonyl)phenyl, 3-(N,N-dimethylaminomethyl)-4-methoxyphenyl, 3-(N,N-dimethylaminomethyl)-5-methoxyphenyl, 2-(N,N-dimethylaminomethyl)-4-methoxyphenyl, 2-(N,N-dimethylaminomethyl)-5-methoxyphenyl, 2-(N,N-dimethylaminomethyl)-6-methoxyphenyl, 2-(N,N-dimethylaminomethyl)-3-methoxyphenyl, 4-(N,N-dimethylaminomethyl)-3-methoxyphenyl, 3-(morpholinomethyl)phenyl, 2-(morpholinomethyl)phenyl, 4-(morpholinomethyl)phenyl, 3-cyano-4-methoxyphenyl, 2-cyano-4-methoxyphenyl, 3-cyano-5-methoxyphenyl, 2-cyano-5-methoxyphenyl, 2-cyano-6-methoxyphenyl, 2-cyano-3-methoxyphenyl, 4-cyano-3-methoxyphenyl, 4-aminomethyl-3-methylphenyl, 2-aminomethyl-4-methylphenyl, 3-aminomethyl-5-methylphenyl, 3-aminomethyl-4-methylphenyl, 2-aminomethyl-5-methylphenyl, 2-aminomethyl-6-methylphenyl, 2-aminomethyl-3-methylphenyl, (1-methyl)cyclopropyl, (2-methyl)cyclopropyl, 1-fluorocyclopropyl, 4-(2-methyl)pyridinyl, 3-(4-methyl)-pyridinyl, 2-(4-methyl)-pyridinyl, 2-(5-methyl)-pyridinyl, 2-(6-methyl)-pyridinyl, 2-(3-methyl)-pyridinyl, 2-(3-acetamido)-pyridinyl, 2-(4-acetamido)-pyridinyl, 2-(5-acetamido)-pyridinyl, 2-(6-acetamido)-pyridinyl, 3-(2-acetamido)-pyridinyl, 3-(4-acetamido)-pyridinyl, 3-(5-acetamido)-pyridinyl, 3-(6-acetamido)-pyridinyl, 4-(2-acetamido)-pyridinyl, 4-(3-acetamido)-pyridinyl, 4-(N-methylsulfamoyl)phenyl, 3-(N-methylsulfamoyl)phenyl, 2-(N-methylsulfamoyl)phenyl, 4-(N,N-dimethylsulfamoyl)phenyl, 3-(N,N-dimethylsulfamoyl)phenyl, 2-(N,N-dimethylsulfamoyl)phenyl, 3-(N-methylsulfamoyl)pyrrolyl, 3-(N,N-dimethylsulfamoyl)pyrrolyl, 4-(N-methylamido)phenyl, 3-(N-methylamido)phenyl, 2-(N-methylamido)phenyl, 4-(N-methylaminomethyl)phenyl, 3-(N-methylaminomethyl)phenyl, 2-(N-methylaminomethyl)phenyl, 3-(N-methylaminomethyl)-4-methoxyphenyl, 3-(N-methylaminomethyl)-5-methoxyphenyl, 2-(N-methylaminomethyl)-4-methoxyphenyl, 2-(N-methylaminomethyl)-5-methoxyphenyl, 2-(N-methylaminomethyl)-6-methoxyphenyl, 4-(N-methylaminomethyl)-3-methoxyphenyl, 2-(N-methylaminomethyl)-3-methoxyphenyl, 4-(acetylamino)phenyl, 3-(acetylamino)phenyl, 2-(acetylamino)phenyl, and ethynyl, 2-(5-N,N-dimethylaminomethyl)thiophenyl, 5-(2-methyl)indazolyl, 5-(3-methyl)indazolyl, 5-(7-methyl)indazolyl, 5-1H-indazolyl, 6-1H-indazolyl, 3-(1-methyl)pyrrolyl, 3-(2-methoxycarbonyl)pyrrolyl, 4-(2-methoxy)pyridinyl, 4-(1H-pyrrolo[2,3-b]pyridinyl), 5-(1H-pyrrolo[2,3-b]pyridinyl), 2-methyl-5-(1H-pyrrolo[2,3-b]pyridinyl), 4-(pyrazol-1-yl)phenyl, 4-(1H-pyrazol-5-yl)phenyl, 4-(1H-pyrazol-4yl)phenyl, 4-(1H-pyrazol-3-yl)phenyl, 4-carboxy-3-methylphenyl, 3-1H-pyrazolyl, 4-1H-pyrazolyl, 5-1H pyrazolyl, 4-1H-benzimidazolyl, 5-1H-benzimidazolyl, 1-methyl-5-benzimidazolyl, 2-methyl-5-1H-benzimidazolyl, 1-methyl-6-benzimidazolyl, 2-hydroxy-5-1H-benzimidazolyl, 5-(2-methyl)-benzoxazolyl, 5-(1-methyl)indolyl, 5-(3-methyl)indolyl, 4-1H-indazolyl, 3-(hydroxymethyl)phenyl, 3-hydroxyphenyl, 1,3-benzodioxol-5-yl and 1,2,3-benzotriazol-6-yl, 3-methyl-5-(1H-pyrazolo[3,4-b]pyridinyl, 1-methyl-5-(1H-pyrrazolo[3,4-b]pyridinyl, 2-amino-5-pyrimidinyl, 1,5-naphthyl-3-yl, 1,5-naphthyridin-3-yl, 5-benzofuranyl, 6-(2-methyl)-benzothiazolyl, 5-(2-methyl)-benzothiazolyl, 5-benzoxazolyl, 6-benzoxazolyl, 6-(2-methyl)-benzoxazolyl, 5-(2-methyl)-benzoxazolyl, 4-((2-methoxyethoxy)methyl)phenyl, 4-(cyclopropylmethoxy)methyl)phenyl, 3-(2-(aminomethyl)-1,5-dimethyl)-pyrrolyl, 5-oxoisoindolinyl, 3-fluoro-4-pyrrolidin-1-yl-phenyl, 4-(1-aminocarbonylmethyl)-pyrazolyl, 4-(1-oxetan-3-yl)-pyrazolyl, 4-(1-amino-2-methyl-2-propyl)phenyl, 4-1-(pyrrolidin-1-yl)ethyl)phenyl, 4-(1-dimethylamino)ethyl)phenyl, 4-(2-hydroxypropan-2-yl)phenyl, 4-(2-methyl, 1-methylamino-propan-2-yl)phenyl, 4-(2-methyl, 1-dimethylamino-propan-2-yl)phenyl, 4-(1-amino-2-hydroxypropan-2-yl)phenyl and 3-dimethylaminoethyl-4-methoxyphenyl.

The compound may have the following Formula (III):

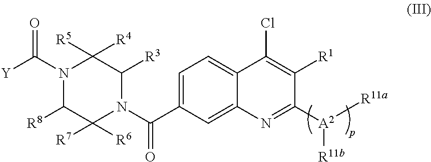

R¹ and R^{11a} may be independently selected from the group consisting of H, halogen, cyano, optionally substituted alkyl, optionally substituted alkene, optionally substituted alkyne, optionally substituted alkoxy, optionally substituted amino, optionally substituted cycloalkyl, optionally substituted heterocycloalkyl, optionally substituted aryl and optionally substituted heteroaryl.

R^{11b} may be absent, H or optionally substituted alkyl.

A² may be selected from CH, N, O or S; and

p may be an integer selected from 0, 1 or 2.

When p is 0, the A² linked group may represent R^{11a} or R^{11b}. When p is 0, the A² linked group may represent R^{11a}.

The compound may have the following Formula (IIIa):

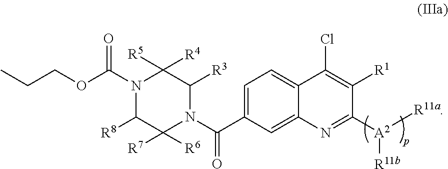

R¹ and R^{11a} may be independently selected from the group consisting of a bond, H, cyano, methyl, ethyl, ethynyl, phenyl, 2-methylphenyl, 3-methylphenyl, 4-methylphenyl, 2-hydroxyphenyl, 3-hydroxyphenyl, 4-hydroxyphenyl, 2-(trifluoromethyl)phenyl, 3-(trifluoromethyl)phenyl, 4-(trifluoromethyl)phenyl, 2-chlorophenyl, 3-chlorophenyl, 4-chlorophenyl, 2-fluorophenyl, 3-fluorophenyl, 4-fluorophenyl, 2-fluoromethylphenyl, 3-fluoromethylphenyl, 4-fluoromethylphenyl, 2-hydroxymethylphenyl, 3-hydroxymethylphenyl, 4-hydroxymethylphenyl, 2-methoxyphenyl, 3-methoxyphenyl, 4-methoxyphenyl, 2-ethoxyethylphenyl, 3-ethoxyethylphenyl, 4-ethoxyethylphenyl, 2-(azidomethyl)phenyl, 3-(azidomethyl)phenyl, 4-(azidomethyl)phenyl, 2,3-difluorophenyl, 2,4-difluorophenyl, 2,5-difluorophenyl, 2,6-difluorophenyl, 3,4-difluorophenyl, 3,5-difluorophenyl, 3,5-difluoro-4-hydroxyphenyl, 3,5-difluoro-4-(aminocarbonyl)phenyl, 3,5-difluoro-4-aminomethylphenyl, 2-cyanophenyl, 3-cyanophenyl, 4-cyanophenyl, 2-(cyanomethyl)phenyl, 3-(cyanomethyl)phenyl, 4-(cyanomethyl)phenyl, 2-nitrophenyl, 3-nitrophenyl, 4-nitrophenyl, 2-aminophenyl, 3-aminophenyl, 4-aminophenyl, 2-(aminomethyl)phenyl, 3-(aminomethyl)phenyl, 4-(aminomethyl)phenyl, 2-(dimethylamino)phenyl, 3-(dimethylamino)phenyl, 4-(dimethylamino)phenyl, 2-(aminocarbonyl)phenyl, 3-(aminocarbonyl)phenyl, 4-(aminocarbonyl)phenyl, 2-(methylaminocarbonyl)phenyl, 3-(methylaminocarbonyl)phenyl, 4-(methylaminocarbonyl)phenyl, 2-(ethylaminocarbonyl)phenyl, 3-(ethylaminocarbonyl)phenyl, 4-(ethylaminocarbonyl)phenyl, 4-(1-ethoxyethyl)phenyl, 4-(2-hydroxy-2-propyl)phenyl, 2-pyridinyl, 3-pyridinyl, 4-pyridinyl, 2-methyl-3-pyridinyl, 4-methyl-3-pyridinyl, 5-methyl-3-pyridinyl, 6-methyl-3-pyridinyl, 2-thiophenyl, 3-thiophenyl, 1-pyrrolidinyl, 2-pyrrolidinyl, 3-pyrrolidinyl, 1-pyrrolyl, 2-pyrrolyl, 3-pyrrolyl, 1-methyl-3-pyrrolyl, 3-(1,2,5-trimethyl)-pyrrolyl, 2-ethynylphenyl, 3-ethynylphenyl, 4-ethynylphenyl, 2-ethylphenyl, 3-ethylphenyl, 4-ethylphenyl, 2-(1-hydroxyethyl)phenyl, 3-(1-hydroxyethyl)phenyl, 4-(1-hydroxyethyl)phenyl, 2-(2-hydroxyethyl)phenyl, 3-(2-hydroxyethyl)phenyl, 4-(2-hydroxyethyl)phenyl, 4-fluoro-(3-methyl)phenyl, 4-fluoro-(2-methyl)phenyl, 3-fluoro-(2-methyl)phenyl, 3-fluoro-(4-methyl)phenyl, 3-fluoro-(5-methyl)phenyl, 2-fluoro-(5-methyl)phenyl, 4-fluoro-(3-methoxy)phenyl, 4-fluoro-(2-methoxy)phenyl, 3-fluoro-(2-methoxy)phenyl, 3-fluoro-(4-methoxy)phenyl, 3-fluoro-(5-methoxy)phenyl, 2-fluoro-(5-methoxy)phenyl, 4-fluoro-3-hydroxyphenyl, 4-fluoro-2-hydroxyphenyl, 4-hydroxy-3-fluorophenyl, 4-hydroxy-2-fluorophenyl, 4-fluoro-3-hydroxymethylphenyl, 4-fluoro-2-hydroxymethylphenyl, 3-fluoro-2-hydroxymethylphenyl, 3-fluoro-4-hydroxymethylphenyl, 3-fluoro-5-hydroxymethylphenyl, 2-fluoro-5-hydroxymethylphenyl, 3-fluoro-4-(2-hydroxy-2-propyl)phenyl, 3-(aminocarbonyl)-4-fluorophenyl, 2-(aminocarbonyl)-4-fluorophenyl, 2-(aminocarbonyl)-3-fluorophenyl, 4-(aminocarbonyl)-3-fluorophenyl, 5-(aminocarbonyl)-3-fluorophenyl, 5-(aminocarbonyl)-2-fluorophenyl, 4-fluoro-3-(methylaminocarbonyl)phenyl, 3-fluoro-4-(methylaminocarbonyl)phenyl, 4-fluoro-2-(methylaminocarbonyl)phenyl, 3-fluoro-2-(methylaminocarbonyl)phenyl, 4-(cyclopropylaminocarbonyl)phenyl, 2-(cyclopropylaminocarbonyl)phenyl, 3-(cyclopropylaminocarbonyl)phenyl, 4-fluoro-3-(cyclopropylaminocarbonyl)phenyl, 3-fluoro-4-(cyclopropylaminocarbonyl)phenyl, 4-fluoro-2-(cyclopropylaminocarbonyl)phenyl, 3-fluoro-2-(cyclopropylaminocarbonyl)phenyl, (3-fluoro-4-(dimethylaminocarbonyl)phenyl, 3-fluoro-5-(dimethylaminocarbonyl)phenyl, 2-fluoro-5-(dimethylaminocarbonyl)phenyl, 4-fluoro-3-(dimethylaminocarbonyl)phenyl, 4-fluoro-2-(dimethylaminocarbonyl)phenyl, 3-fluoro-2-(dimethylaminocarbonyl)phenyl, 3-methyl-4-(methylaminocarbonyl)phenyl, 3-amino-4-fluorophenyl,2-amino-4-fluorophenyl, 3-aminomethyl-4-fluorophenyl, 2-aminomethyl-4-fluorophenyl, 3-hydroxymethyl-4-methylphenyl, 2-hydroxymethyl-4-methyl-phenyl, 2-hydroxymethyl-3-methyl-phenyl, 4-hydroxymethyl-3-methylphenyl, 5-hydroxymethyl-3-methylphenyl, 5-hydroxymethyl-2-methylphenyl, 2-morpholinophenyl, 3-morpholinophenyl, 4-morpholinophenyl, 2-(1-pyrrolidinyl)phenyl, 3-(1-pyrrolidinyl)phenyl, 4-(1-pyrrolidinyl)phenyl, 4-(1-amino-1-cyclopropyl)phenyl, cyclopropyl, cyclobutyl, cyclopentyl, cyclohexyl, 2-thiazolyl, 4-thiazolyl, 5-thiazolyl, 2-methylthiazolyl, 4-methylthiazolyl, 4-(dimethylamido)phenyl, 2-(dimethylamido)phenyl, 3-(dimethylamido)phenyl, 2-benzylamin, 3-benzylamin, 4-benzylamin, 2-methylaminophenyl, 3-methylaminophenyl, 4-methylaminophenyl, 6-(1-methyl)indazolyl, 6-(2-methyl)indazolyl, 5-(1-methyl)indazolyl, 5-(2-methyl)indazolyl, 4-(1-methyl)indazolyl, 3-(1-methyl)indazolyl, 5-(3-methyl)indazolyl, 5-(1-methyl)pyrazolyl, 4-(1-methyl)-pyrazolyl, 3-(1-methyl)pyrazolyl, 4-(1-isopropyl)-pyrazolyl, 4-(1-difluoromethyl)-pyrazolyl, 4-(5-trifluoromethyl)-pyrazolyl, 4-(1-(2,2,2)-trifluoroethyl)pyrazolyl, 4-(1-cyclopentyl)pyrazolyl, 2-(1-methyl)pyrazolylphenyl, 3-(1-methyl)pyrazolylphenyl, 1-imidazolyl, 2-imidazolyl, 3-imidazolyl, 4-(imidazol-1-yl)phenyl, 4-(4-methylpiperazino)phenyl, 3-(4-methyl)piperazino)phenyl, 2-(4-methyl)piperazino)phenyl, 3-[1,2,4]-triazol-4-ylphenyl, 2-[1,2,4]-triazol-4-ylphenyl, 4-[1,2,4]-triazol-4-ylphenyl, 3-(aminomethyl)-4-methoxyphenyl, 3-(aminomethyl)-5-methoxyphenyl, 2-(aminomethyl)-4-methoxyphenyl, 2-(aminomethyl)-5-methoxyphenyl, 2-(aminomethyl)-6-methoxyphenyl, 4-(aminomethyl)-3-methoxyphenyl, 2-(aminomethyl)-3-methoxyphenyl, 4-(dimethylaminomethyl)phenyl, 3-(dimethylaminomethyl)phenyl, 2-(dimethylaminomethyl)phenyl, 4-fluoro-3-(dimethylaminomethyl)phenyl, 4-fluoro-2-(dimethylaminomethyl)phenyl, 4-methoxy-3-methylphenyl, 2-methoxy-4-methylphenyl, 3-methoxy-5-methylphenyl, 3-methoxy-4-methylphenyl, 2-methoxy-5-methylphenyl, 2-methoxy-6-methylphenyl, 2-methoxy-3-methylphenyl, 4-methoxy-3-hydroxymethylphenyl, 3-methoxy-4-hydroxymethylphenyl, 2-methoxy-4-hydroxymethylphenyl, 3-methoxy-5-hydroxymethylphenyl, 2-methoxy-5-hydroxymethylphenyl, 2-methoxy-6-hydroxymethylphenyl, 2-methoxy-3-hydroxymethylphenyl, 3-cyano-4-methylphenyl, 4-cyano-3-methylphenyl, 2-cyano-4-methylphenyl, 3-cyano-5-methylphenyl, 2-cyano-5-methylphenyl, 2-cyano-6-methylphenyl, 2-cyano-3-methylphenyl, 4-(aminosulfonyl)phenyl, 3-(aminosulfonyl)phenyl, 2-(aminosulfonyl)phenyl, 3-(N,N-dimethylaminomethyl)-4-methoxyphenyl, 3-(N,N-dimethylaminomethyl)-5-methoxyphenyl, 2-(N,N-dimethylaminomethyl)-4-methoxyphenyl, 2-(N,N-dimethylaminomethyl)-5-methoxyphenyl, 2-(N,N-dimethylaminomethyl)-6-methoxyphenyl, 2-(N,N-dimethylaminomethyl)-3-methoxyphenyl, 4-(N,N-dimethylaminomethyl)-3-methoxyphenyl, 3-(morpholinomethyl)phenyl, 2-(morpholinomethyl)phenyl, 4-(morpholinomethyl)phenyl, 3-cyano-4-methoxyphenyl, 2-cyano-4-methoxyphenyl, 3-cyano-5-methoxyphenyl, 2-cyano-5-methoxyphenyl, 2-cyano-6-methoxyphenyl, 2-cyano-3-methoxyphenyl, 4-cyano-3-methoxyphenyl, 4-aminomethyl-3-methylphenyl, 2-aminomethyl-4-methylphenyl, 3-aminomethyl-5-methylphenyl, 3-aminomethyl-4-methylphenyl, 2-aminomethyl-5-methylphenyl, 2-aminomethyl-6-methylphenyl, 2-aminomethyl-3-methylphenyl, (1-methyl)cyclopropyl, (2-methyl)cyclopropyl, 1-fluorocyclopropyl, 4-(3-methyl)pyridinyl, 3-(4-methyl)pyridinyl, 2-(4-methyl)pyridinyl, 4-(2-methyl)pyridinyl, 2-(5-methyl)pyridinyl, 2-(6-methyl)pyridinyl, 2-(3-methyl)pyridinyl, 2-(3-acetamido)-pyridinyl, 2-(4-acetamido)-pyridinyl, 2-(5-acetamido)-pyridinyl, 2-(6-acetamido)-pyridinyl, 3-(2-acetamido)-pyridinyl, 3-(4-acetamido)-pyridinyl, 3-(5-acetamido)-pyridinyl, 3-(6-acetamido)-pyridinyl, 4-(2-acetamido)-pyridinyl, 4-(3-acetamido)-pyridinyl, 4-(N-methylsulfamoyl)phenyl, 3-(N-methylsulfamoyl)phenyl, 2-(N-methylsulfamoyl)phenyl, 4-(N-methylamido)phenyl, 3-(N-methylamido)phenyl, 2-(N-methylamido)phenyl, 4-(N,N-dimethylsulfamoyl)phenyl, 3-(N,N-dimethylsulfamoyl)phenyl, 2-(N,N-dimethylsulfamoyl)phenyl, 3-(N-methylsulfamoyl)pyrrolyl, 3-(N,N-dimethylsulfamoyl)pyrrolyl, 4-(N-methylaminomethyl)phenyl, 3-(N-methylaminomethyl)phenyl, 2-(N-methylaminomethyl)phenyl, 3-(N-methylaminomethyl)-4-methoxyphenyl, 3-(N-methylaminomethyl)-5-methoxyphenyl, 2-(N-methylaminomethyl)-4-methoxyphenyl, 2-(N-methylaminomethyl)-5-methoxyphenyl, 2-(N-methylaminomethyl)-6-methoxyphenyl, 4-(N-methylaminomethyl)-3-methoxyphenyl, 2-(N-methylaminomethyl)-3-methoxyphenyl, 4-(acetylamino)phenyl, 3-(acetylamino)phenyl, 2-(acetylamino)phenyl, 4-(N,N-dimethylsulfamoyl)phenyl, 3-(N,N-dimethylsulfamoyl)phenyl, 2-(N,N-dimethylsulfamoyl)phenyl, 2-(5-N,N-dimethylaminomethyl)thiophenyl, 5-(2-methyl)indazolyl, 5-(3-methyl)indazolyl, 5-(7-methyl)indazolyl, 5-1H-indazolyl, 6-1H-indazolyl, 3-(1-methyl)pyrrolyl, 3-(2-methoxycarbonyl)pyrrolyl, 4-(2-methoxy)pyridinyl, 4-(1H-pyrrolo [2,3-b]pyridinyl), 5-(1H-pyrrolo[2,3-b]pyridinyl), 2-methyl-5-(1H-pyrrolo[2,3-b]pyridinyl) , 4-(pyrazol-1-yl)phenyl, 4-(1H-pyrazol-5-yl)phenyl, 4-(1H-pyrazol-4-yl)phenyl, 4-(1H-pyrazol-3-yl)phenyl, 4-carboxy-3-methylphenyl, 3-1H-pyrazolyl, 4-1H-pyrazolyl, 5-1H pyrazolyl, 4-1H-benzimidazolyl, 5-1H-benzimidazolyl, 1-methyl-5-1H-benzimidazolyl, 2-methyl-5-1H-benzimidazolyl, 1-methyl-6-1H-benzimidazolyl, 2-hydroxy-5-1H-benzimidazolyl, 5-(2-methyl)-benzoxazolyl, 5-(1-methyl)indolyl, 5-(3-methyl)indolyl, 4-1H-indazolyl, 3-(hydroxymethyl)phenyl, 3-hydroxyphenyl, 1,3-benzodioxol-5-yl and 1,2,3-benzotriazol-6-yl, 3-methyl-5-(1H-pyrazolo[3,4-b]pyridinyl, 1-methyl-5-(1H-pyrrazolo[3,4-b]pyridinyl, 2-amino-5-pyrimidinyl, 1,5-naphthyl-3-yl, 5-benzofuran, 6-(2-methyl)-benzothiazolyl, 5-(2-methyl)-benzothiazolyl, 5-benzoxazolyl, 6-benzoxazolyl, 6-(2-methyl)-benzoxazol, 4-((2-methoxyethoxy)methyl)phenyl, 4-(cyclopropylmethoxy)methyl)phenyl, 3-(2-(aminomethyl)-1,5-dimethyl)-pyrrolyl, 5-oxoisoindolinyl, 3-fluoro-4-pyrrolidin-1-yl-phenyl, 4-(1-aminocarbonylmethyl)-pyrazolyl, 4-(1-oxetan-3-yl)-pyrazolyl, 4-(1-amino-2-methyl-2-propyl)phenyl, 4-1-(pyrrolidin-1-yl)ethyl)phenyl, and 4-(1-dimethylamino)ethyl)phenyl, 4-(2-hydroxypropan-2-yl)phenyl, 4-(2-methyl, 1-methylamino-propan-2-yl)phenyl, 4-(2-methyl, 1-dimethylamino-propan-2-yl)phenyl, 4-(1-amino-2-hydroxypropan-2-yl)phenyl and 3-dimethylaminoethyl-4-methoxyphenyl.

The compound may have the following Formula (IIIa′):

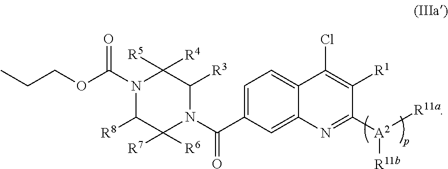

R¹ and R^{11a} may be independently selected from the group consisting of bond, H, cyano, methyl, ethyl, phenyl, 2-methylphenyl, 3-methylphenyl, 4-methylphenyl, 2-(trifluoromethyl)phenyl, 3-(trifluoromethyl)phenyl, 4-(trifluoromethyl)phenyl, 2-chlorophenyl, 3-chlorophenyl, 4-chlorophenyl, 2-fluorophenyl, 3-fluorophenyl, 4-fluorophenyl, 2-methoxyphenyl, 3-methoxyphenyl, 4-methoxyphenyl, 2,3-difluorophenyl, 2,4-difluorophenyl, 2,5-difluorophenyl, 2,6-difluorophenyl, 3,4-difluorophenyl, 3,5-difluorophenyl, 2-cyanophenyl, 3-cyanophenyl, 4-cyanophenyl, 2-(cyanomethyl)phenyl, 3-(cyanomethyl)phenyl, 4-(cyanomethyl)phenyl, 2-nitrophenyl, 3-nitrophenyl, 4-nitrophenyl, 2-aminophenyl, 3-aminophenyl, 4-aminophenyl, 2-(aminomethyl)phenyl, 3-(aminomethyl)phenyl, 4-(aminomethyl)phenyl, 2-(dimethylamino)phenyl, 3-(dimethylamino)phenyl, 4-(dimethylamino)phenyl, 2-(aminocarbonyl)phenyl, 3-(aminocarbonyl)phenyl, 4-(aminocarbonyl)phenyl, 2-pyridinyl, 3-pyridinyl, 4-pyridinyl, 5-methyl-3-pyridinyl, thiophenyls such as 2-thiophenyl, 1-pyrrolidinyl, 2-pyrrolidinyl, 3-pyrrolidinyl, 2-ethynylphenyl, 3-ethynylphenyl, 4-ethynylphenyl, 2-ethylphenyl, 3-ethylphenyl, 4-ethylphenyl, 4-fluoro-(3-methyl)phenyl, 4-fluoro-(2-methyl)phenyl, 3-fluoro-(2-methyl)phenyl, 3-fluoro-(4-methyl)phenyl, 3-fluoro-(5-methyl)phenyl, 2-fluoro-(5-methyl)phenyl, 4-fluoro-(3-methoxy)phenyl, 4-fluoro-(2-methoxy)phenyl, 3-fluoro-(2-methoxy)phenyl, 3-fluoro-(4-methoxy)phenyl, 3-fluoro-(5-methoxy)phenyl, 2-fluoro-(5-methoxy)phenyl, 2-morpholinophenyl, 3-morpholinophenyl, 4-morpholinophenyl, 2-(1-pyrrolidinyl)phenyl, 3-(1-pyrrolidinyl)phenyl, 4-(1-pyrrolidinyl)phenyl, cyclopropyl, cyclobutyl, cyclopentyl, cyclohexyl, 2-thiazolyl, 4-thiazolyl, 4-(dimethylamido)phenyl, 2-(dimethylamido)phenyl, 3-(dimethylamido)phenyl, 2-methylaminophenyl, 3-methylaminophenyl, 4-methylaminophenyl, 5-(1-methyl)indazolyl, 4-(1-methyl)indazolyl, 3-(1-methyl)indazolyl, 5-(1-methyl)pyrazolyl, 3-(1-methyl)pyrazolyl, 2-(1-methyl)pyrazolylphenyl, 3-(1-methyl)pyrazolylphenyl, 1-imidazolyl, 2-imidazolyl, 3-imidazolyl, 4-(imidazol-1-yl)phenyl, 4-(N,N-dimethylsulfamoyl)phenyl, 3-(N,N-dimethylsulfamoyl)phenyl, 2-(N,N-dimethylsulfamoyl)phenyl, 4-(4-methylpiperazino)phenyl, 3-(4-methyl)piperazino)phenyl, 2-(4-methyl)piperazino)phenyl, 3-[1,2,4]-triazol-4-ylphenyl, 2-[1,2,4]-triazol-4-ylphenyl, 4-[1,2,4]-triazol-4-ylphenyl, 3-(aminomethyl)-4-methoxyphenyl, 3-(aminomethyl)-5-methoxyphenyl, 2-(aminomethyl)-4-methoxyphenyl, 2-(aminomethyl)-5-methoxyphenyl, 2-(aminomethyl)-6-methoxyphenyl, 4-(aminomethyl)-3-methoxyphenyl, 2-(aminomethyl)-3-methoxyphenyl, 4-(dimethylaminomethyl)phenyl, 3-(dimethylaminomethyl)phenyl, 2-(dimethylaminomethyl)phenyl, 4-methoxy-3-methylphenyl, 2-methoxy-4-methylphenyl, 3-methoxy-5-methylphenyl, 3-methoxy-4-methylphenyl, 2-methoxy-5-methylphenyl, 2-methoxy-6-methylphenyl, 2-methoxy-3-methylphenyl, 4-methoxy-3-hydroxymethylphenyl, 3-methoxy-4-hydroxymethylphenyl, 2-methoxy-4-hydroxymethylphenyl, 3-methoxy-5-hydroxymethylphenyl, 2-methoxy-5-hydroxymethylphenyl, 2-methoxy-6-hydroxymethylphenyl, 2-methoxy-3-hydroxymethylphenyl, 3-cyano-4-methylphenyl, 4-cyano-3-methylphenyl, 2-cyano-4-methylphenyl, 3-cyano-5-methylphenyl, 2-cyano-5-methylphenyl, 2-cyano-6-methylphenyl, 2-cyano-3-methylphenyl, 4-(aminosulfonyl)phenyl, 3-(aminosulfonyl)phenyl, 2-(aminosulfonyl)phenyl, 3-(N,N-dimethylaminomethyl)-4-methoxyphenyl, 3-(N,N-dimethylaminomethyl)-5-methoxyphenyl, 2-(N,N-dimethylaminomethyl)-4-methoxyphenyl, 2-(N,N-dimethylaminomethyl)-5-methoxyphenyl, 2-(N,N-dimethylaminomethyl)-6-methoxyphenyl, 2-(N,N-dimethylaminomethyl)-3-methoxyphenyl, 4-(N,N-dimethylaminomethyl)-3-methoxyphenyl, 3-(morpholinomethyl)phenyl, 2-(morpholinomethyl)phenyl, 4-(morpholinomethyl)phenyl, 3-cyano-4-methoxyphenyl, 2-cyano-4-methoxyphenyl, 3-cyano-5-methoxyphenyl, 2-cyano-5-methoxyphenyl, 2-cyano-6-methoxyphenyl, 2-cyano-3-methoxyphenyl, 4-cyano-3-methoxyphenyl, 4-aminomethyl-3-methylphenyl, 2-aminomethyl-4-methylphenyl, 3-aminomethyl-5-methylphenyl, 3-aminomethyl-4-methylphenyl, 2-aminomethyl-5-methylphenyl, 2-aminomethyl-6-methylphenyl, 2-aminomethyl-3-methylphenyl, (1-methyl)cyclopropyl, (2-methyl)cyclopropyl, 4-(3-methyl)pyridinyl, 3-(4-methyl)pyridinyl, 2-(4-methyl)pyridinyl, 4-(2-methyl)pyridinyl, 2-(5-methyl)pyridinyl, 2-(6-methyl)pyridinyl, 2-(3-methyl)pyridinyl, 4-(N-methylsulfamoyl)phenyl, 3-(N-methylsulfamoyl)phenyl, 2-(N-methylsulfamoyl)phenyl, 4-(N-methylamido)phenyl, 3-(N-methylamido)phenyl, 2-(N-methylamido)phenyl, 4-(N,N-dimethylsulfamoyl)phenyl, 3-(N,N-dimethylsulfamoyl)phenyl, 2-(N,N-dimethylsulfamoyl)phenyl, 4-(N-methylaminomethyl)phenyl, 3-(N-methylaminomethyl)phenyl, 2-(N-methylaminomethyl)phenyl, 3-(N-methylaminomethyl)-4-methoxyphenyl, 3-(N-methylaminomethyl)-5-methoxyphenyl, 2-(N-methylaminomethyl)-4-methoxyphenyl, 2-(N-methylaminomethyl)-5-methoxyphenyl, 2-(N-methylaminomethyl)-6-methoxyphenyl, 4-(N-methylaminomethyl)-3-methoxyphenyl, 2-(N-methylaminomethyl)-3-methoxyphenyl, 4-(acetylamino)phenyl, 3-(acetylamino)phenyl, 2-(acetylamino)phenyl, 4-(N,N-dimethylsulfamoyl)phenyl, 3-(N,N-dimethylsulfamoyl)phenyl, 2-(N,N-dimethylsulfamoyl)phenyl, 2-(5-N,N-dimethylaminomethyl)thiophenyl, 5-(2-methyl)indazolyl, 5-1H-indazolyl, 6-1H-indazolyl, 3-(1-methyl)pyrrolyl, 4-(2-methoxy)pyridinyl, 5-(1H-pyrrolo[2,3-b]pyridinyl), 4-(pyrazol-1-yl)phenyl, 4-(1H-pyrazol-5-yl)phenyl, 4-(1H-pyrazol-4-yl)phenyl, 4-(1H-pyrazol-3-yl)phenyl, 4-carboxy-3-methylphenyl, 3-1H-pyrazolyl, 5-1H-benzimidazolyl, 5-(1-methyl)indolyl, 4-1H-indazolyl, 3-(hydroxymethyl)phenyl and 3-hydroxyphenyl and ethynyl.

In some embodiments, R¹ and R² may be taken together to form an optionally substituted 5-membered cycloalkyl, an optionally substituted 6-membered cycloalkyl, an optionally substituted 5-membered heterocycloalkyl or an optionally substituted 6-membered heterocycloalkyl.

The compound may have the following Formula (IV):

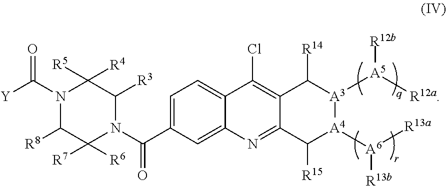

A³ and A⁴ may be independently selected from CH or N.

A⁵ and A⁶ may be independently selected from CH, N, O or S.

R^{12a}, R^{13a}, R¹⁴ and R¹⁵ may be independently selected from the group consisting of H, halogen, optionally substituted alkyl, optionally substituted alkene, optionally substituted alkyne, optionally substituted alkoxy, optionally substituted amino, optionally substituted acyl, optionally substituted cycloalkyl, optionally substituted heterocycloalkyl, optionally substituted aryl and optionally substituted heteroaryl.

R^{12b} and R^{13b} may be independently absent, H or an optionally substituted alkyl; and

q and r may be independently integers selected from 0, 1 or 2.

When q is 0, the A⁵ linked group may represent R^{12a} or R^{12b}. When q is 0, the A⁵ linked group may represent R^{12a}.

When r is 0, the A⁶ linked group may represent R^{13a} or R^{13b}. When r is 0, the A⁶ linked group may represent R^{13a}.

A³ and A⁴ may be both C.

The compound may have the following Formula (IVa):

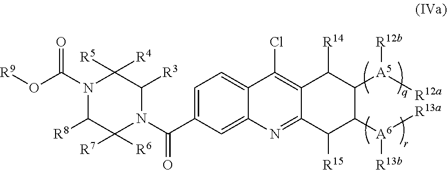

wherein R⁹ may be an optionally substituted C₃ to C₁₀ alkyl, optionally substituted C₃ to C₁₀ alkenyl, optionally substituted C₃ to C₁₀ alkynyl or optionally substituted C₃ to C₇ cycloalkyl.

R^{12a}, R^{13a}, R¹⁴ and R¹⁵ may be independently selected from H, methyl, ethyl, propyl, butyl, halogen, cyano, COOMe, COOEt, phenyl, 2-pyridinyl, 3-pyridinyl, 4-pyridinyl, 2-(3-methyl)pyridinyl, 2-(4-methyl)pyridinyl, 2-(5-methyl)pyridinyl, 2-(6-methyl)pyridinyl, 3-(2-methyl)pyridinyl, 3-(4-methyl)pyridinyl, 3-(5-methyl)pyridinyl, 3-(6-methyl)pyridinyl, 4-(2-methyl)pyridinyl, 4-(3-fluoro)pyridinyl, 2-(3-fluoro)pyridinyl, 2-(4-fluoro)pyridinyl, 2-(5-fluoro)pyridinyl, 2-(6-fluoro)pyridinyl, 3-(2-fluoro)pyridinyl, 3-(4-fluoro)pyridinyl, 3-(5-fluoro)pyridinyl, 3-(6-fluoro)pyridinyl, 4-(2-fluoro)pyridinyl, 4-(3-fluoro)pyridinyl, 2-(3-cyano)pyridinyl, 4-(2-cyano)pyridinyl, 2-(5-cyano)pyridinyl, 2-(6-cyano)pyridinyl, 3-(2-cyano)pyridinyl, 3-(4-cyano)pyridinyl, 3-(5-cyano)pyridinyl, 3-(6-cyano)pyridinyl, 4-(2-cyano)pyridinyl, 2-[3-(aminocarbonyl)]pyridinyl, 2-[4-(aminocarbonyl)]pyridinyl, 2-[5-(aminocarbonyl)]pyridinyl, 2-[6-(aminocarbonyl)]pyridinyl, 3-[2-(aminocarbonye]pyridinyl, 3-[4-(aminocarbonyl)]pyridinyl, 3-[5-(aminocarbonyl)]pyridinyl, 3-[6-(aminocarbonyl)]pyridinyl, 4-[2-(aminocarbonyl)]pyridinyl, 2-[3-(aminomethyl)]pyridinyl, 2-[4-(aminomethyl)]pyridinyl, 2-[5-(aminomethyl)]pyridinyl, 2-[6-(aminomethyl)]pyridinyl, 4-[2-(aminomethyl)]pyridinyl, 3-[4-(aminomethyl)]pyridinyl, 3-[5-(aminomethyl)]pyridinyl, 3-[6-(aminomethyl)]pyridinyl, 4-[2-(aminomethyl)]pyridinyl, 2-pyrimidinyl, 5-pyrimidinyl, 6-pyrimidinyl, 2-thiazolyl, 3-thiazolyl, pyrrolidine, 5-methyl-1,2,4-oxadiazol-3-yl, NH₂, —CH═CH₂, CH₂NH₂, CH₂CH₂NH₂, CH₂N(CH₃)₂, C(O)NH₂, NHC(NH)NH₂,CH₂NHC(NH)NH₂, N(CH₃)₂, CH₂CH═CH₂, ethynyl and 4-(3-methyl)pyrimidinyl, 2-(4-ethynyl)pyridinyl, 3-(4-ethynyl)pyridinyl, 2-(6-ethynyl)pyridinyl, 2-(5-ethynyl)pyridinyl, 3-(4-ethynyl)pyridinyl, 3-(2-ethynyl)pyridinyl, 3-(5-ethynyl)pyridinyl, 3-(6-ethynyl)pyridinyl, 2-(3-cyano)pyrimidinyl, 2-(5-cyano)pyrimidinyl, 2-(6-cyano)pyrimidinyl, 3-(2-cyano)pyrimidinyl, 2-(N-methyl)pyrazolyl, 3-(N-methyl)pyrazolyl, CH₂-pyrrolidine, and CH₂CH₂-pyrrolidine.

R³, R⁴, R⁵, R⁶, R⁷ and R⁸ may be independently selected from the group consisting of a bond, H, methyl, (S)-methyl, (R)-methyl, ethyl, (S)-ethyl, (R)-ethyl, cyano, —CH₂OH, (S)—CH₂OH, (R)—CH₂OH, COOCH₃, (S)—COOCH₃, (R)—COOCH₃, CH₂OC(O)CH₃, (R)—CH₂OC(O)CH₃, (S)—CH₂OC(O)CH₃, CH₂OC(O)CH₂CH₂OCH₃, (R)—CH₂OC(O)CH₂CH₂OCH₃, (S)—CH₂OC(O)CH₂CH₂OCH₃, CH₂CH₂OH, (R)—CH₂CH₂OH, (S)—CH₂CH₂OH, CH₂OC(O)CH₂CH₂CH₃, (R)—CH₂OC(O)CH₂CH₂CH₃, (S)—CH₂OC(O)CH₂CH₂CH₃, CF₃, (R)—CF₃, (S)—CF₃, CH₂OCH₃, (S)—CH₂OCH₃, (R)—CH₂OCH₃, CONHCH₃, (S)—CONHCH₃, (R)—CONHCH₃, CH₂CONHCH₃, (S)—CH₂CONHCH₃, (R)—CH₂CONHCH₃, CH₂COOCH₃, (S)—CH₂COOCH₃, (R)—CH₂COOCH₃, CH₂OC(O)CH(CH₃)₂, (S)—CH₂OC(O)CH(CH₃)₂, (R)—CH₂OC(O)CH(CH₃)₂, CONH₂, (S)—CONH₂, (R)—CONH₂, CH₂CON(CH₃)₂, (S)—CH₂CON(CH₃)₂, (R)—CH₂CON(CH₃)₂, and CH₂C(O)NH(CH₃).

R⁴ and R⁵ or R⁶ and R⁷ may be taken together to form an optionally substituted alkylene bridge or an optionally substituted alkylene bridge wherein one or two alkylene units may be replaced with O, NH or S.

R⁴ and R⁵ or R⁶ and R⁷ may be taken together to form a cyclopropane.

R³ and R⁴, R³ and R⁵, R³ and R⁶, R³ and R⁷, R³ and R⁸, R⁴ and R⁶, R⁴ and R⁷, R⁴ and R⁸, R⁵ and R⁶, R⁵ and R⁷, R⁵ and R⁸, R⁶ and R⁸ or R⁷ and R⁸ may be taken together to form an optionally substituted alkylene bridge or an optionally substituted alkylene bridge wherein one or two alkylene units may be replaced with O, NH or S. The alkylene bridge may be a 1-carbon, 2-carbon or 3-carbon alkylene bridging group wherein —CH— units may optionally be replaced with —NH—, O or S.

For the substituents in Formula (I), (II), (III) and (IV) it can be further stated:

Z¹ and Z² preferably represent O. X preferably represents chlorine, bromine or fluorine. R¹ and R² independently from another preferably represent of a bond, H, cyano, C₁-C₄ alkyl, in each case optionally cyano, fluorine, chlorine, bromine, amino, di(C₁-C₆ alkyl) amino, nitro, carbamoyl, C₁-C₆ alkyl, C₁-C₆ haloalkyl, C₃-C₆ alkene, C₃-C₆ alkyne, C₁-C₄ alkoxy, C₁-C₄ alkyl-CN, C₁-C₄-alkylamino, C₁-C₄ alkyl-CO—NH₂, C₁-C₄ alkyl-NH—C(NH)—NH₂, C₁-C₄ alkyl-C₃-C₆ cycloalkyl, C₁-C₄ alkylamino, C₁-C₄ alkyl-di (C₁-C₃-alkyl) amino, C₁-C₄ alkoxycarbonyl, pyrrolidinyl substituted phenyl, phenylamino, benzyl, pyridinyl, pyridinylamino, pyrimidinyl or pyrimidinylamino.

R¹ and R² preferably may optionally be taken together to form a optionally substituted C₃-C₅ alkylene bridge wherein one —CH₂— unit may be replaced with —NH—, S or O and wherein the optional substituents of the alkylene bridge are selected from cyano, fluorine, chlorine, bromine, carbamoyl, C₁-C₆ alkyl, C₁-C₆ haloalkyl, C₃-C₆ alkene, C₃-C₆ alkyne, C₁-C₄ alkoxy, C₁-C₄ alkyl-CN, C₁-C₄ alkylamino, C₁-C₄ alkyl-CO—NH₂, C₁-C₄ alkyl-NH—C(NH)—NH₂, C₁-C₄ alkyl-C₃-C₆ cycloalkyl, C₁-C₄ alkylamino, C₁-C₄ alkyl-di(C₁-C₃ alkyl) amino, C₁-C₄ alkyl-CO—C₁-C₄ alkyl, C₁-C₄ alkoxycarbonyl; in each case optionally cyano, fluorine, chlorine, bromine, carbamoyl, C₁-C₆ alkyl, C₁-C₆ haloalkyl, C₃-C₆ alkene, C₃-C₆ alkyne, C₁-C₄ alkoxy, C₁-C₄ alkyl-CN, C₁-C₄ alkylamino, C₁-C₄ alkyl-CO—NH₂, C₁-C₄ alkyl-NH—C(NH)—NH₂, C₁-C₄ alkyl-C₃-C₆ cycloalkyl, C₁-C₄ alkylamino, C₁-C₄ alkyl-di (C₁-C₃ alkyl) amino, C₁-C₄ alkoxycarbonyl phenyl, benzyl or pyrrolidinyl; or in each case optionally cyano, fluorine, chlorine, bromine, carbamoyl, C₁-C₆ alkyl, C₁-C₆ haloalkyl, C₃-C₆ alkene, C₃-C₆ alkyne, C₁-C₄ alkoxy, C₁-C₄ alkyl-CN, C₁-C₄ alkylamino, C₁-C₄ alkyl-CO—NH₂, C₁-C₄ alkyl-NH—C(NH)—NH₂, C₁-C₄ alkyl-C₃-C₆cycloalkyl, C₁-C₄ alkylamino, C₁-C₄ alkyl-di(C₁-C₃ alkyl) amino, C₁-C₄ alkoxycarbonyl substituted four or six membered heteroaryl or C₁-C₄ alkyl-heteroaryl containing one to three heteroatoms selected from O, N, or S.

Most preferably R¹ and R² form an optionally substituted —CH₂—CH₂—CH₂—CH₂— bridge.

R³, R⁴, R⁵, R⁶, R⁷ and R⁸ are independently absent, or represent a bond, H, cyano, carbamoyl, —COO— C₁-C₄ alkyl, —CO—NH— C₁-C₄ alkyl, C₁-C₄ alkyl, C₁-C₄ haloalkyl, C₁-C₄ alkyl-OH, C₁-C₄ alkyl-CO—NH₂, C₁-C₄ alkyl-CO— C₁-C₄ alkyl, C₁-C₄ alkyl-CO-di (C₁-C₃ alkyl) amino, C₁-C₄ alkyl-CO—NH—C₁-C₃ alkyl, C₁-C₄ alkyl-O—CO— C₁-C₄ alkyl or C₁-C₄ alkyl-O—CO— C₁-C₃ alkyl-O— C₁-C₃-alkyl. R³, R⁴, R⁵, R⁶, R⁷ or R⁸ may also be taken together to form a C₁-C₄ alkylene bridge. Most preferably two R at the same carbon atom form a —CH₂—CH₂— bridge or two R at different carbon atoms form a —CH₂—CH₂—CH₂— bridge. Y preferably represents R⁹, OR⁹, or NH—R⁹ wherein R⁹ represents C₃-C₆ alkyl, C₃-C₆ alkenyl, optionally substituted C₃-C₁₀ alkynyl or a in each case optionally C₁-C₆ alkyl or C₃-C₆ cycloalkyl substituted isoxazolyl, oxazolyl or pyrazolyl. Y most preferably is OR⁹, if R⁹ is C₃-C₆ alkyl, C₃-C₆ alkenyl, optionally substituted C₃-C₁₀ alkynyl. Y most preferably is R⁹, if R⁹ is a in each case optionally C₁-C₆ alkyl or C₃-C₆ cycloalkyl substituted isoxazolyl, oxazolyl or pyrazolyl.

R⁹ is preferably C₃ to C₉ alkyl, optionally substituted C₃ to C₆ alkenyl or a C₃ to C₉ alkyl, or C₃ to C₇ cycloalkyl substituted oxazolyl, isoxazolyl or pyrazolyl.

R⁹ is more preferably selected from the group consisting of propyl, butyl, pentyl, —CH₂CH(CH₃)₂, —CH₂CH═CH, 5-cyclopropylisoxazol-3-yl, 5-isobutylisoxazol-3-yl, 5-methylisoxazol-3-yl and 5-methylpyrazol-3-yl. R⁹ most preferably is propyl.

Compounds wherein R¹ is preferably methyl or H, most preferably H, may be specifically mentioned.

The compound may be selected from the group consisting of:

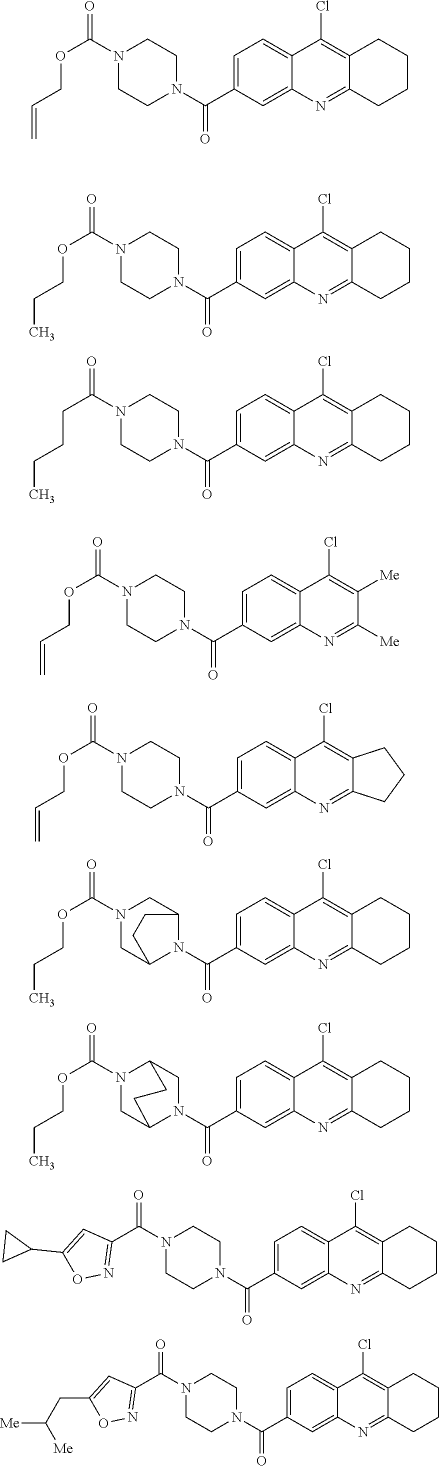

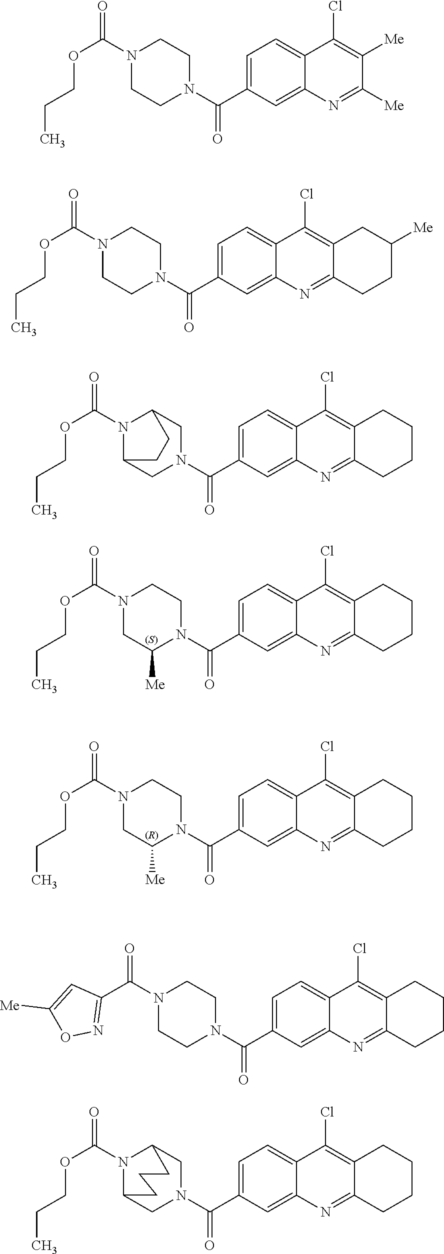

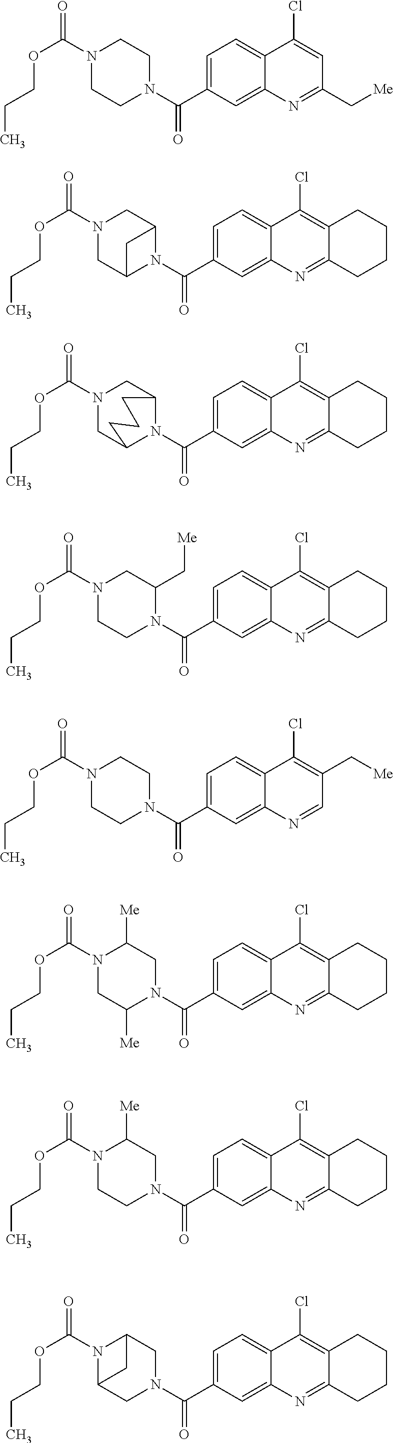

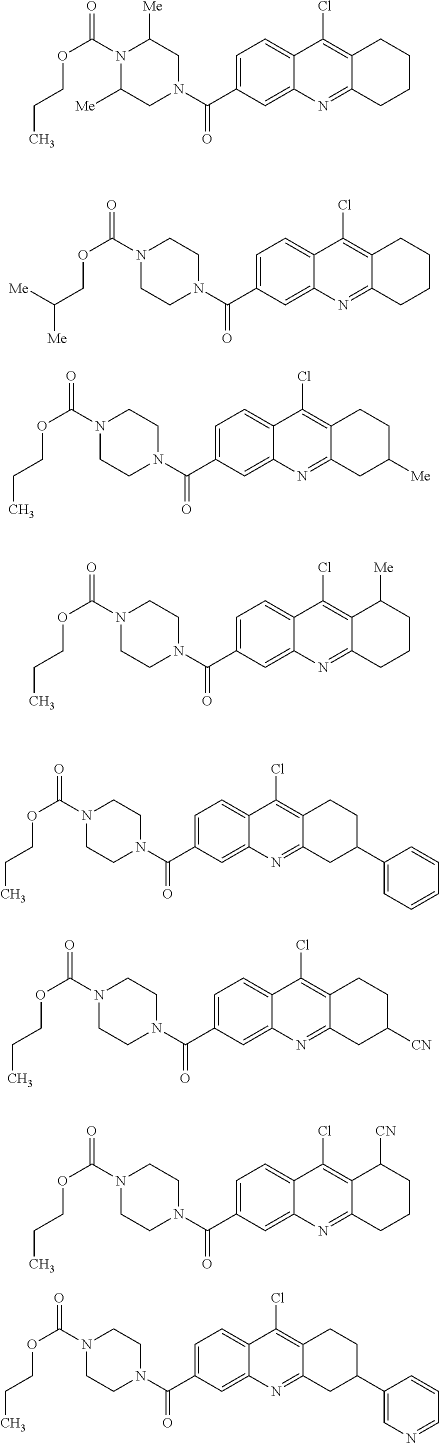

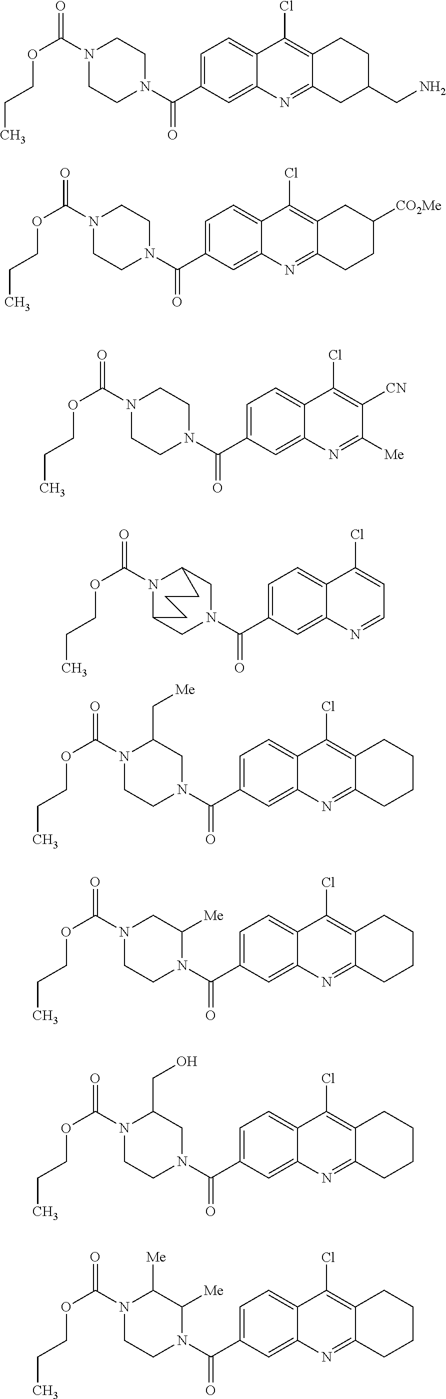

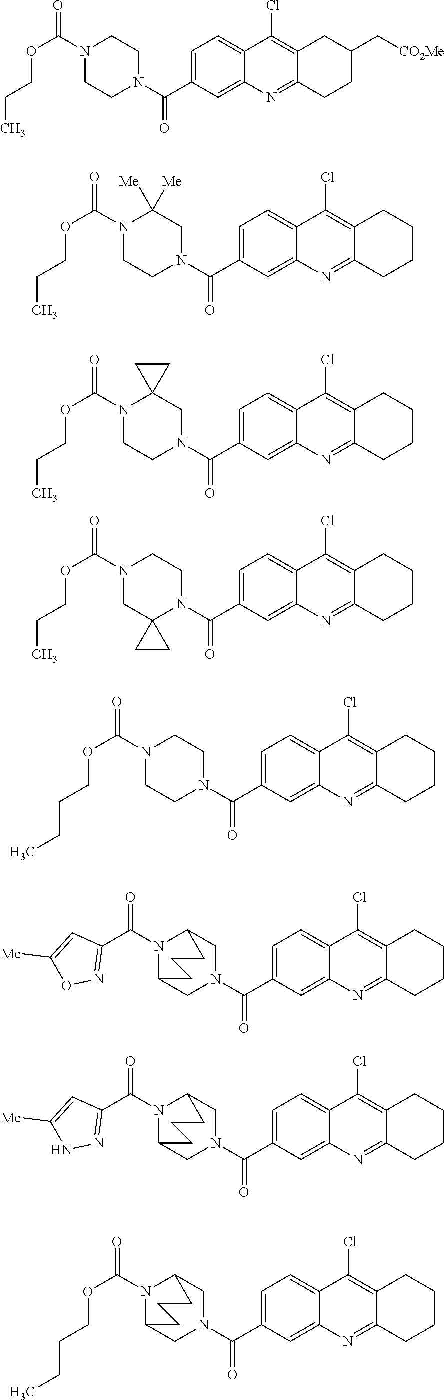

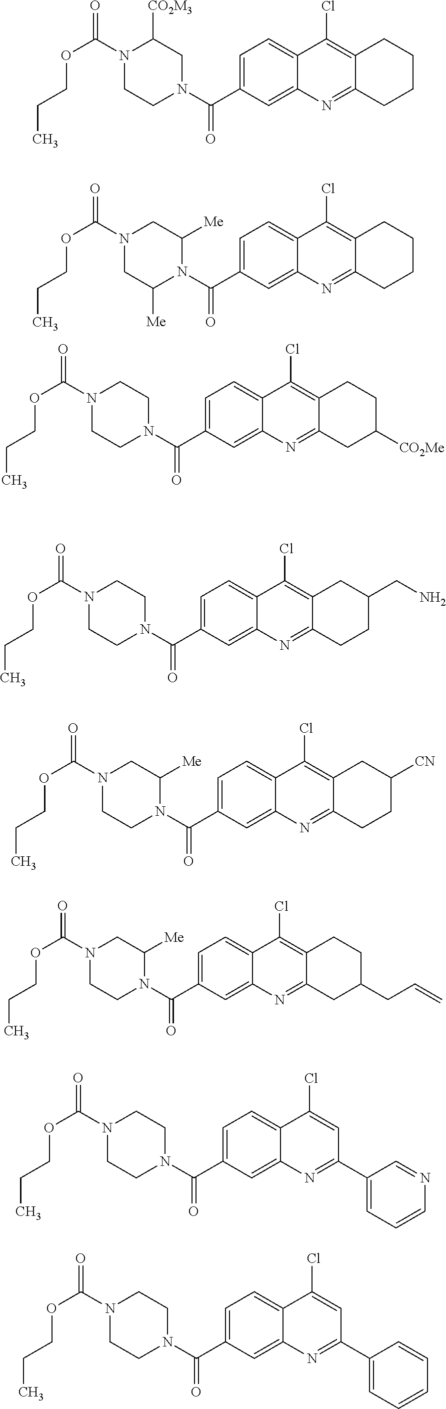

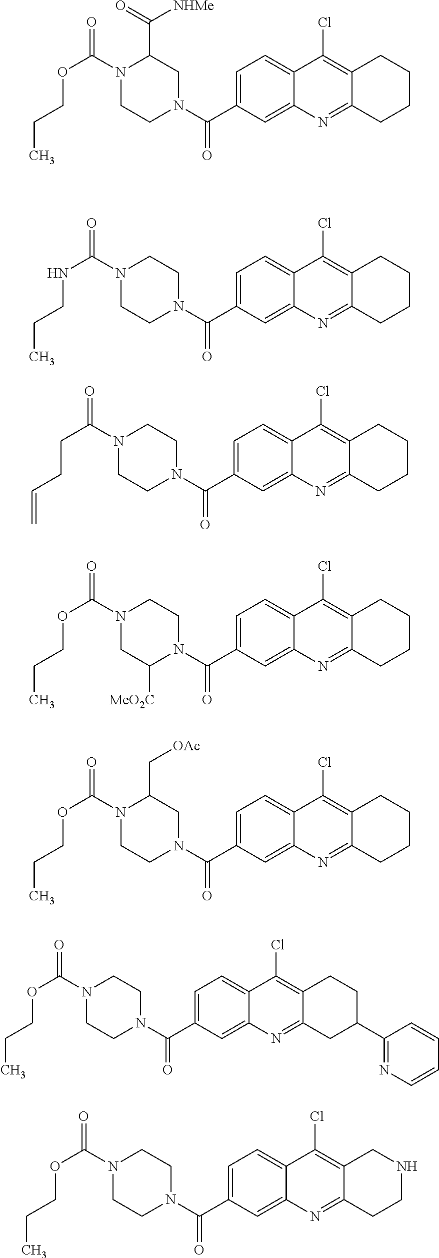

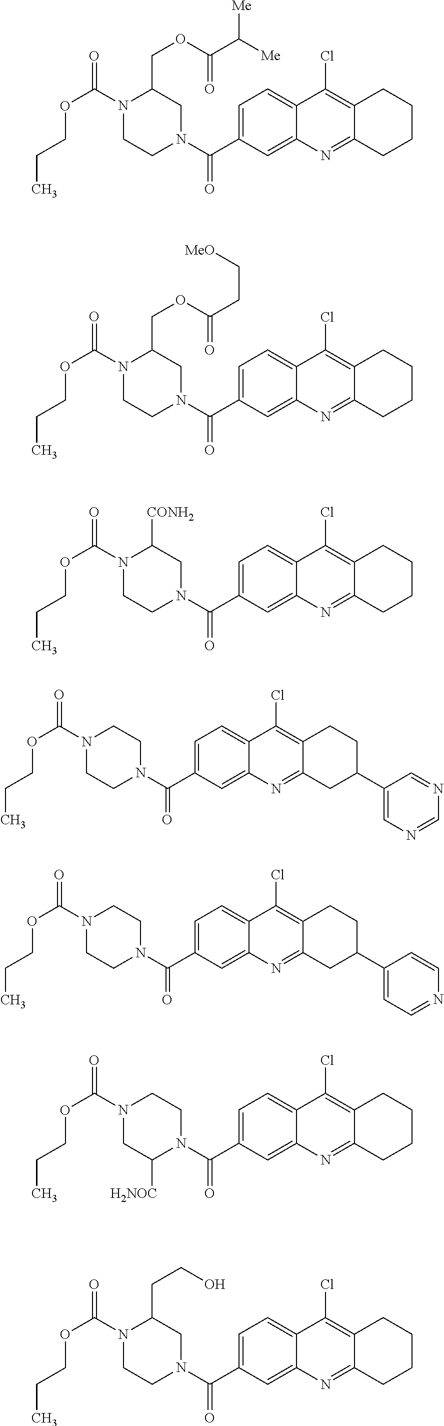

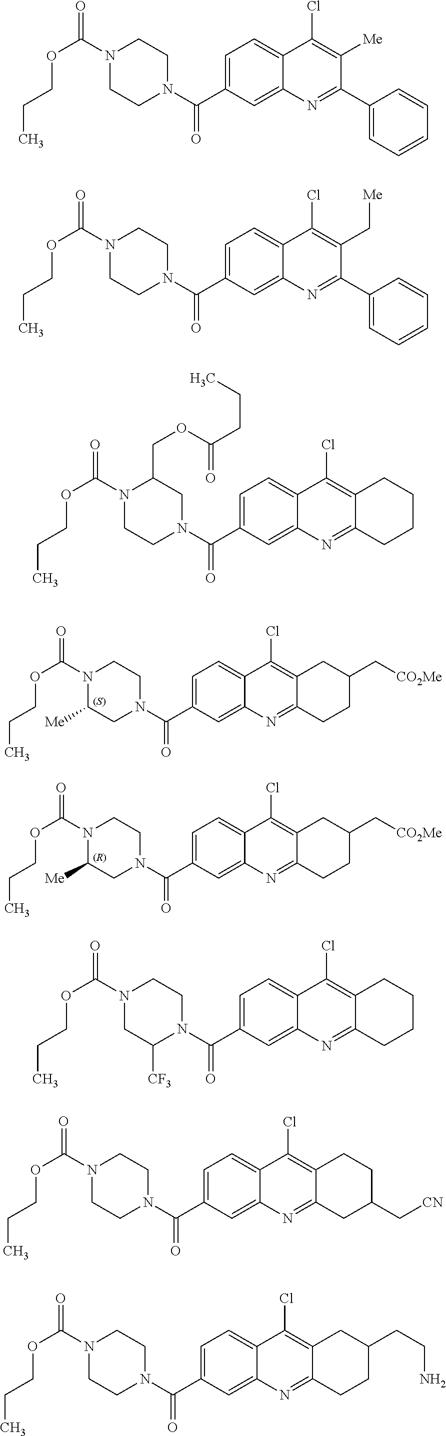

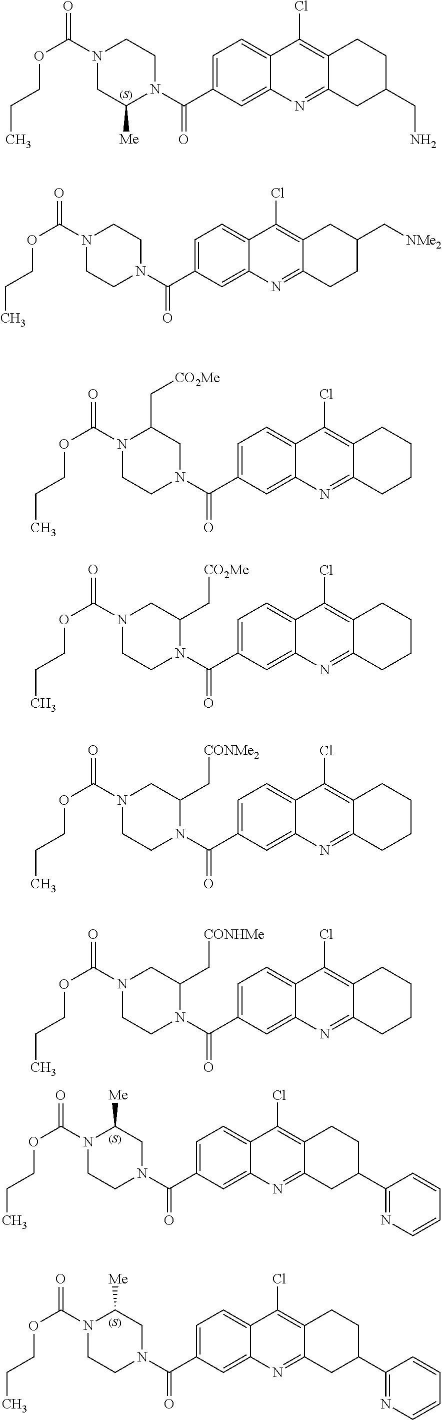

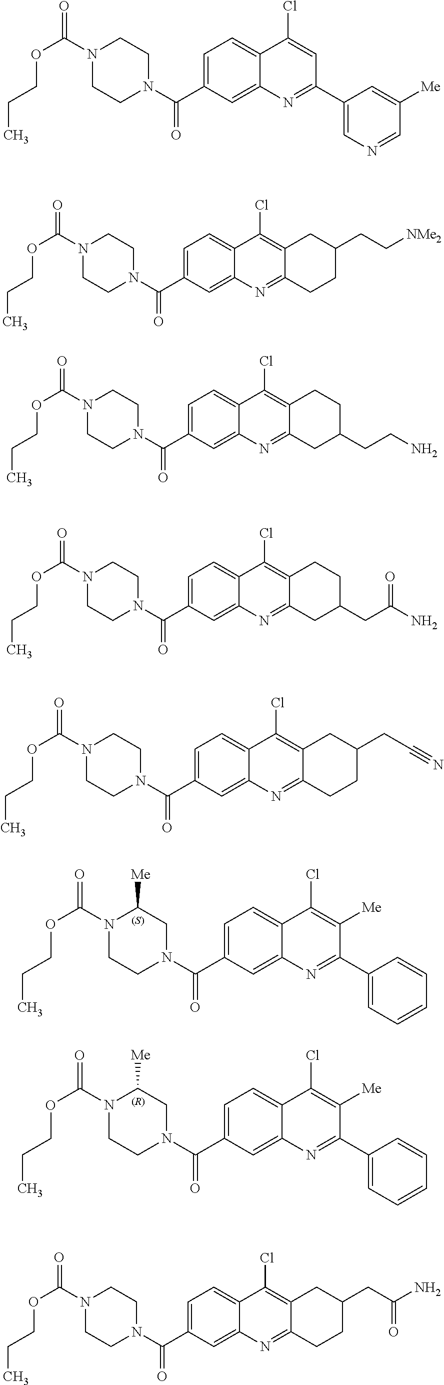

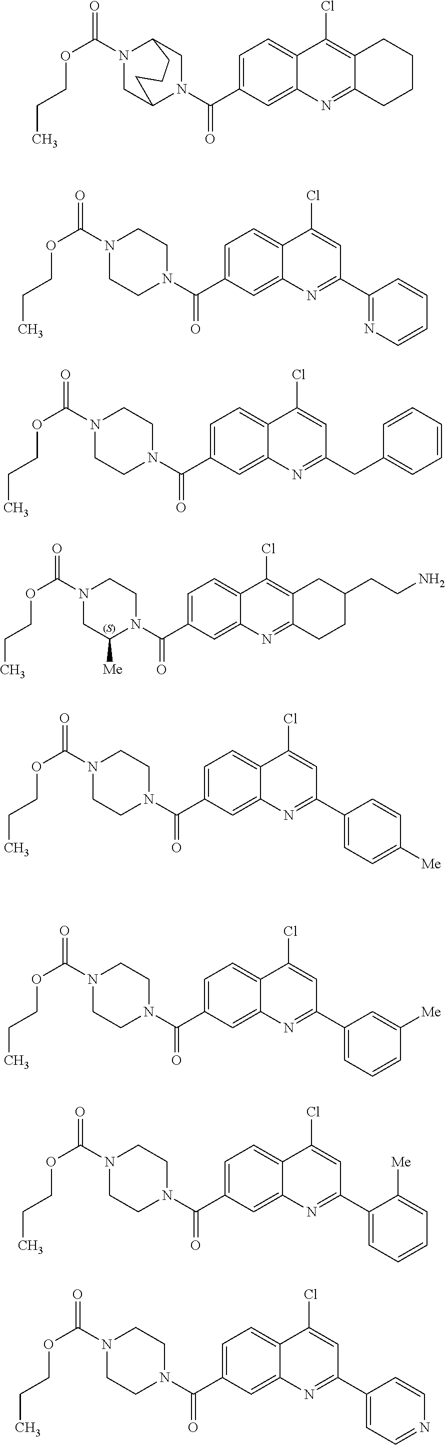

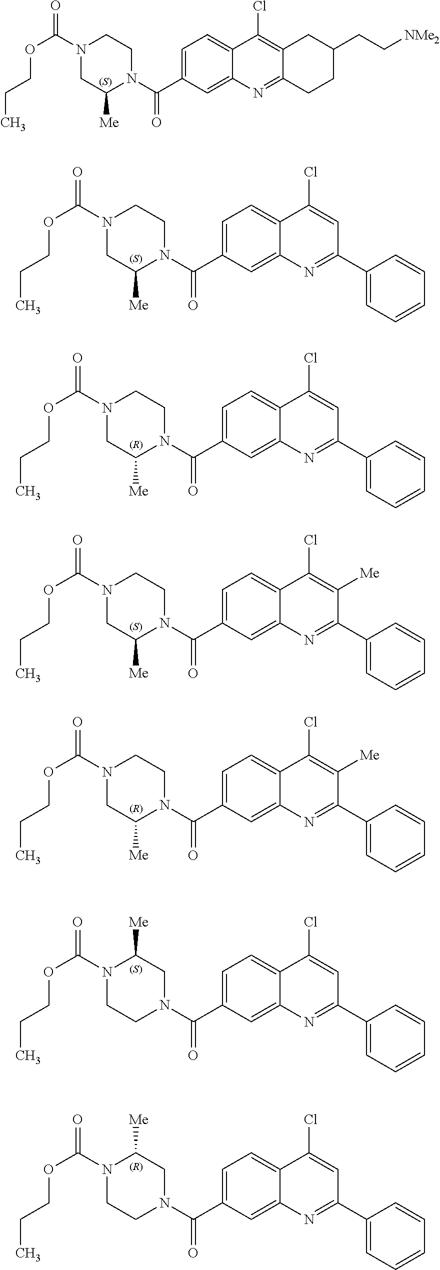

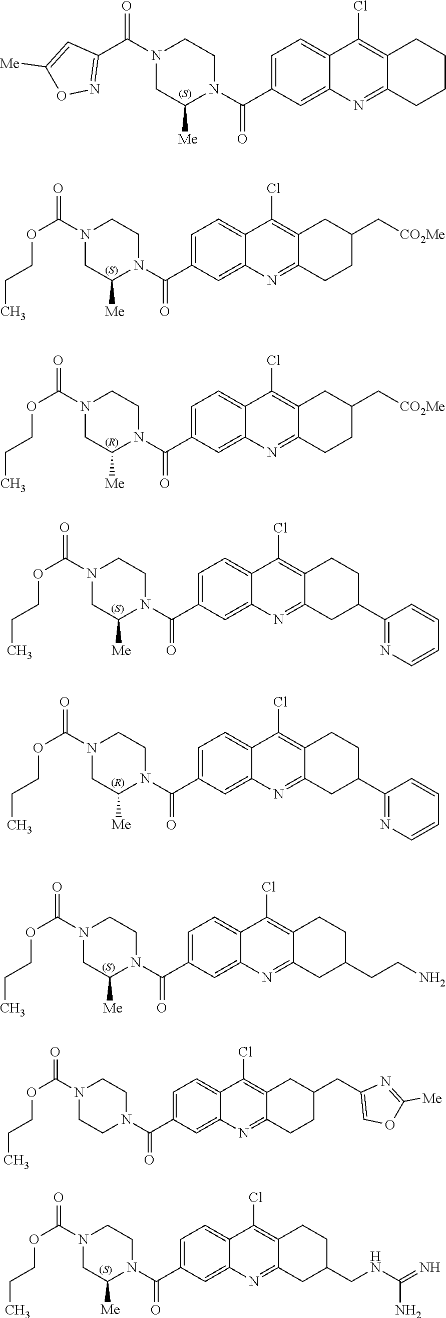

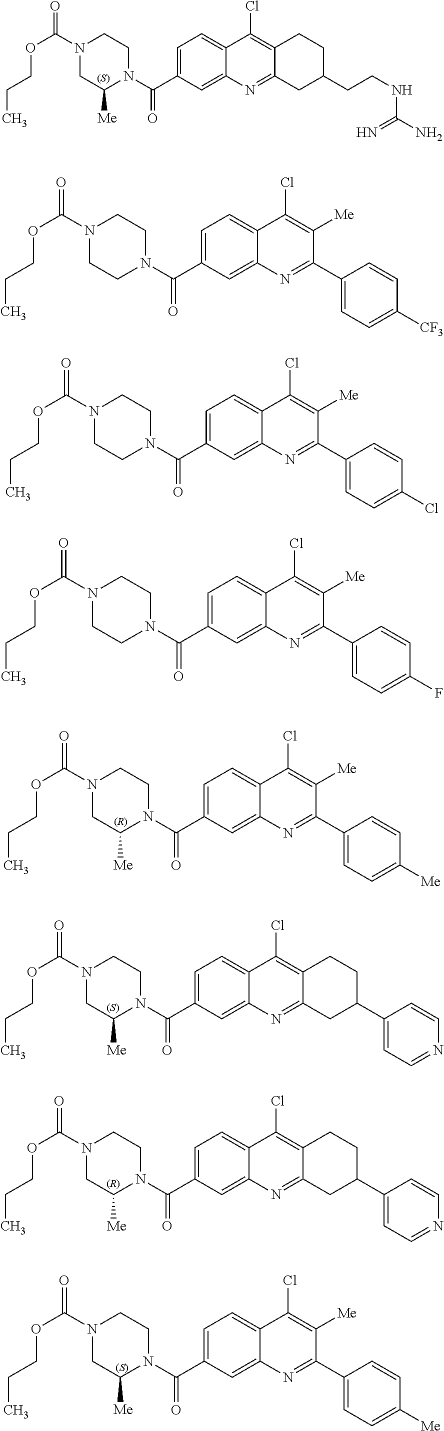

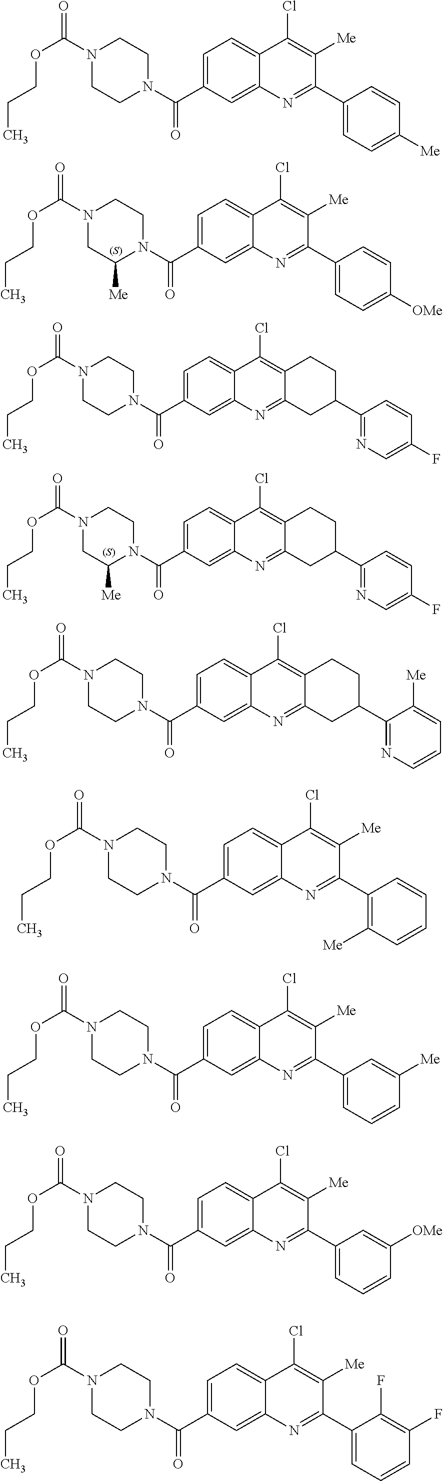

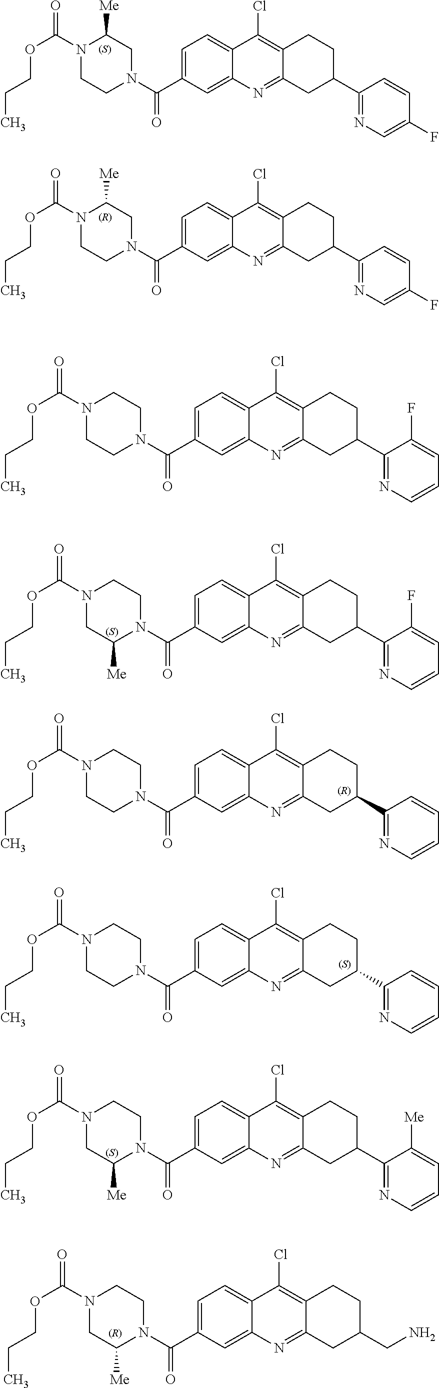

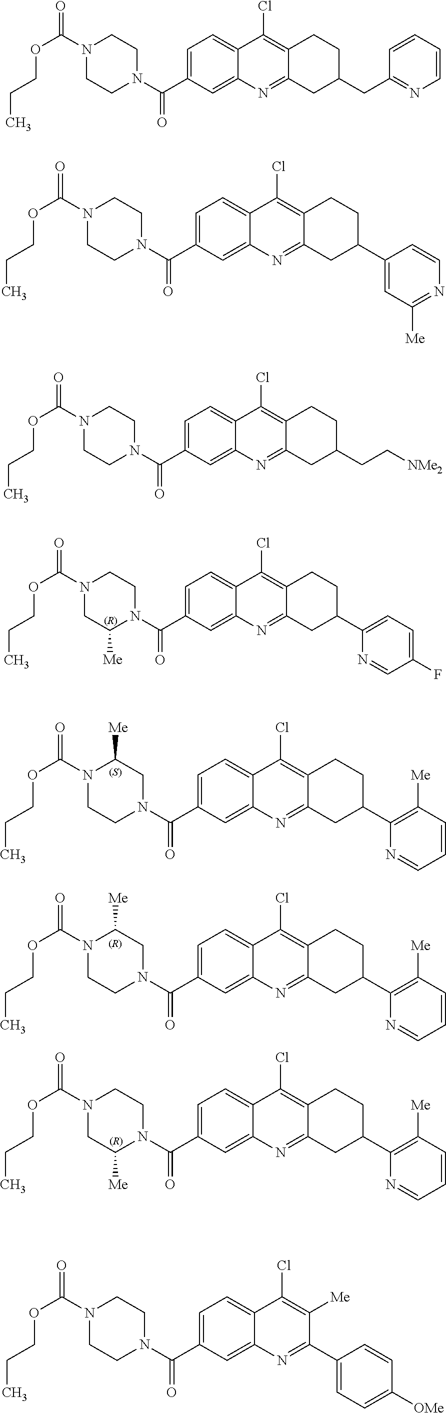

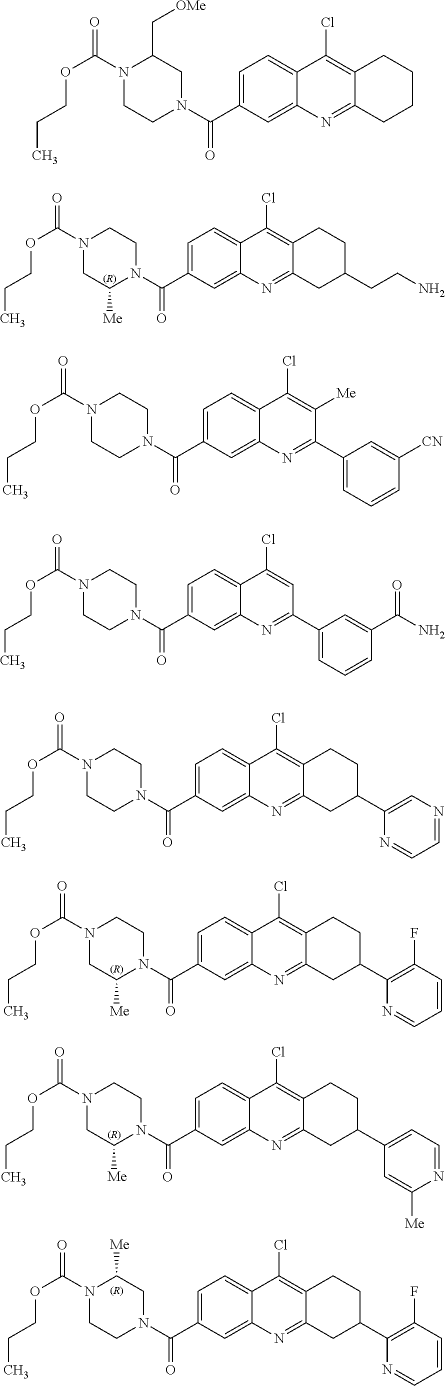

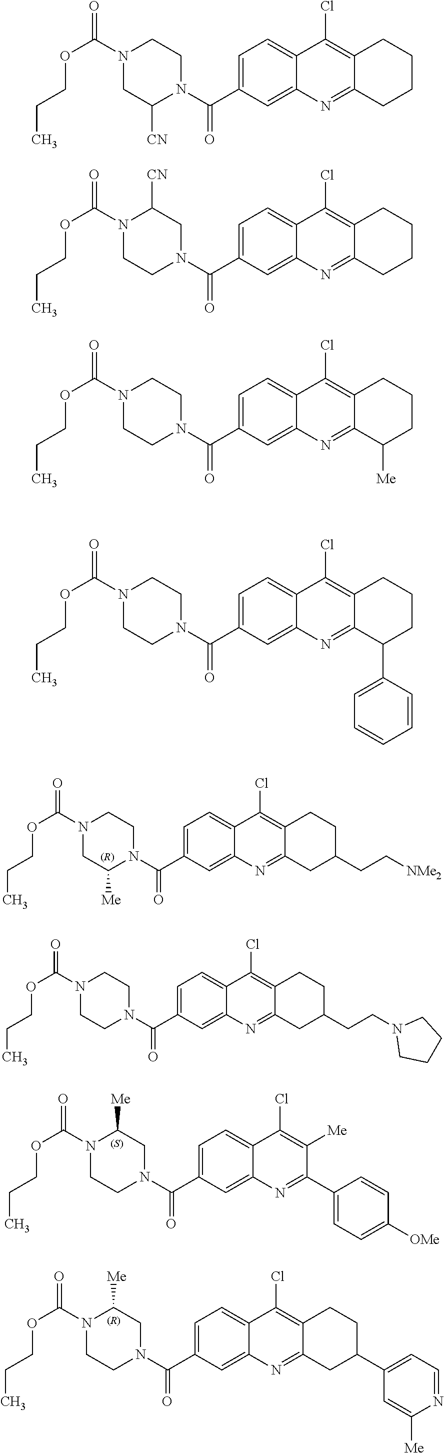

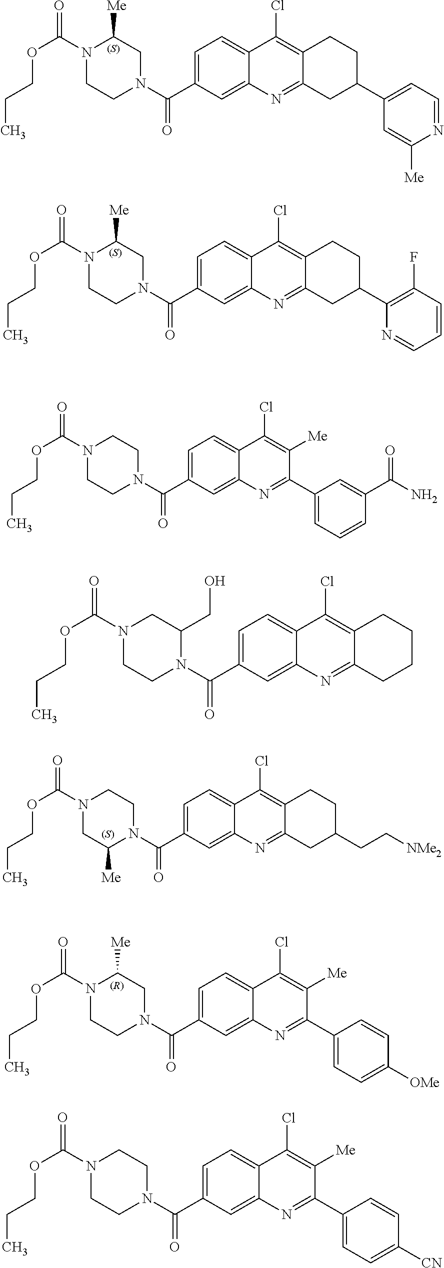

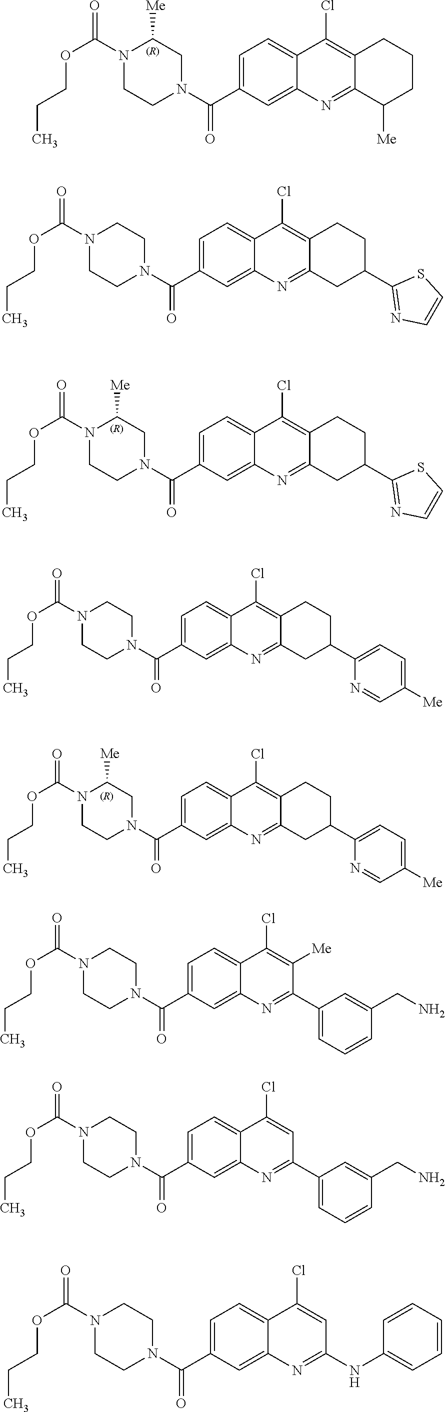

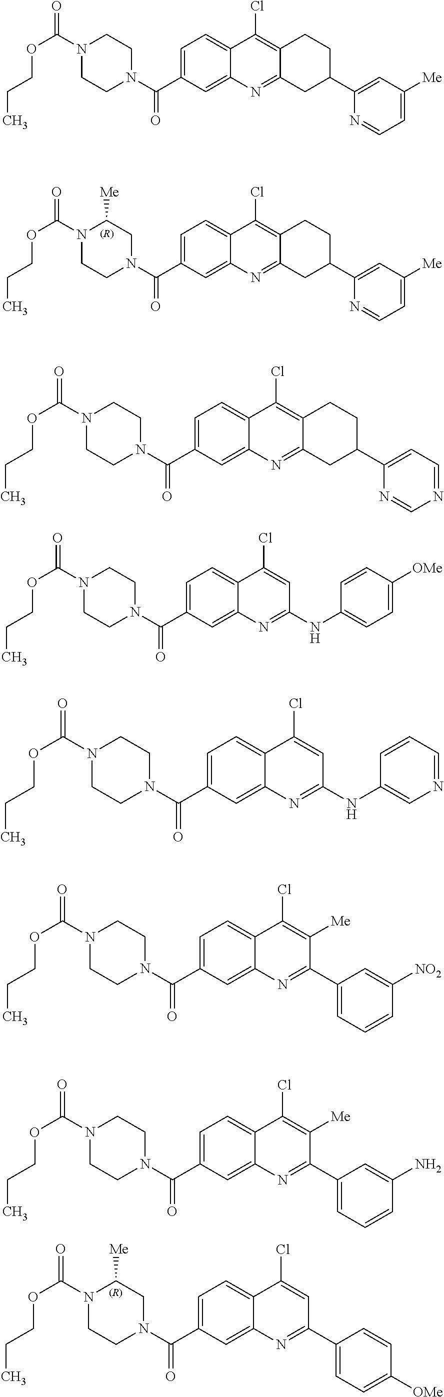

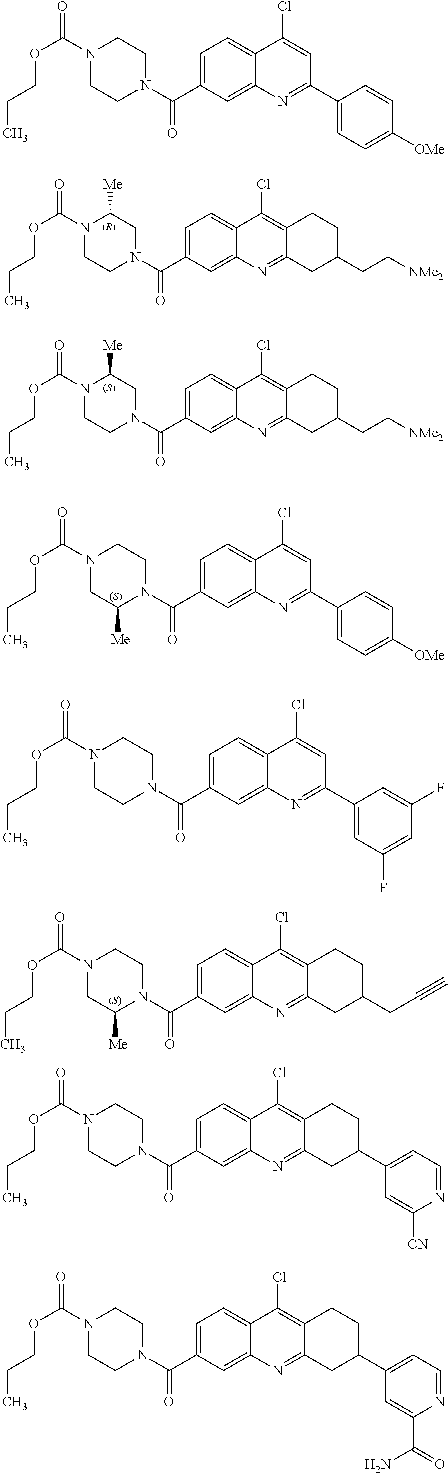

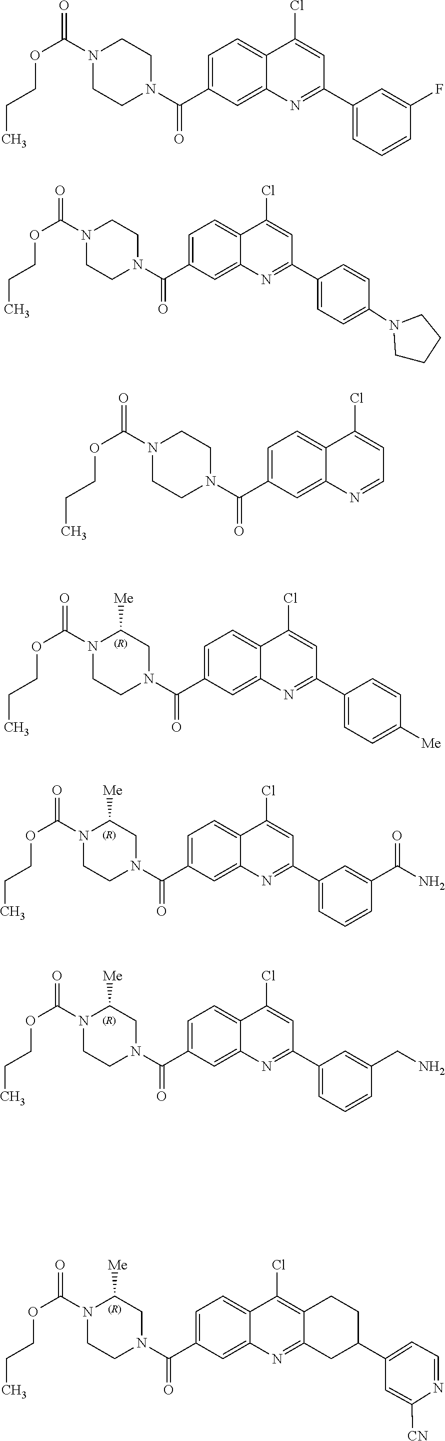

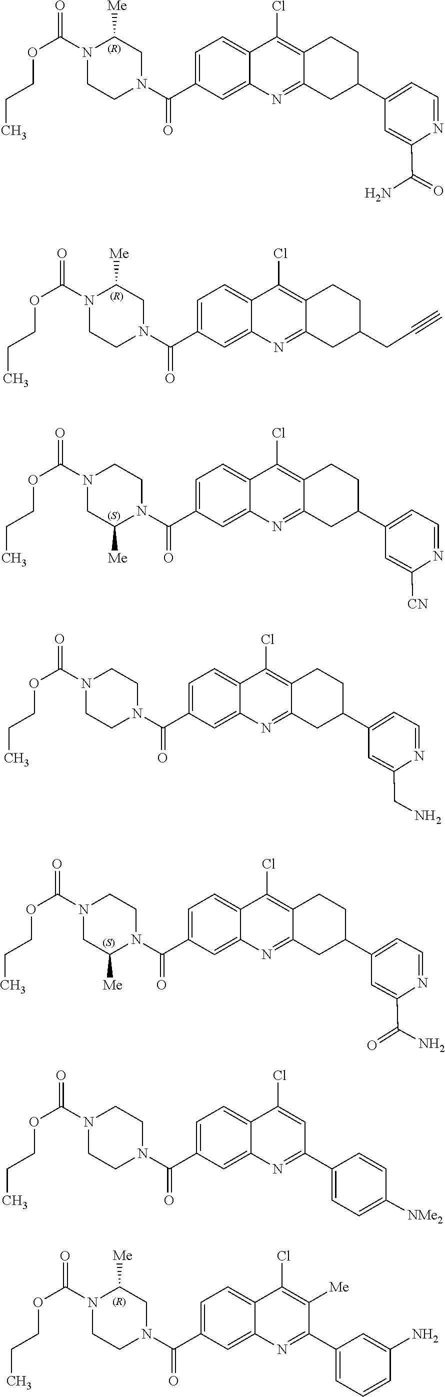

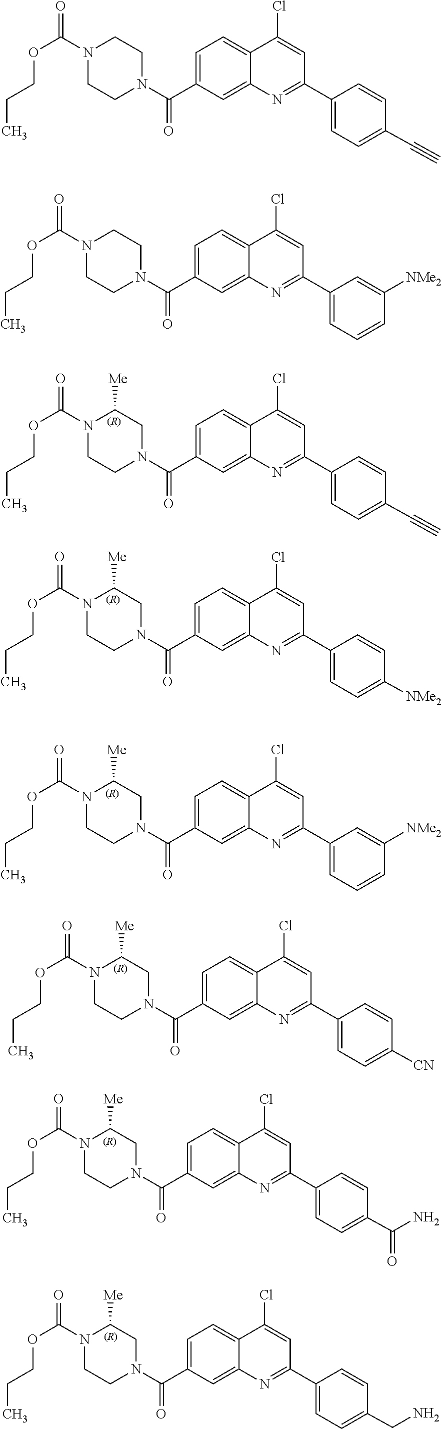

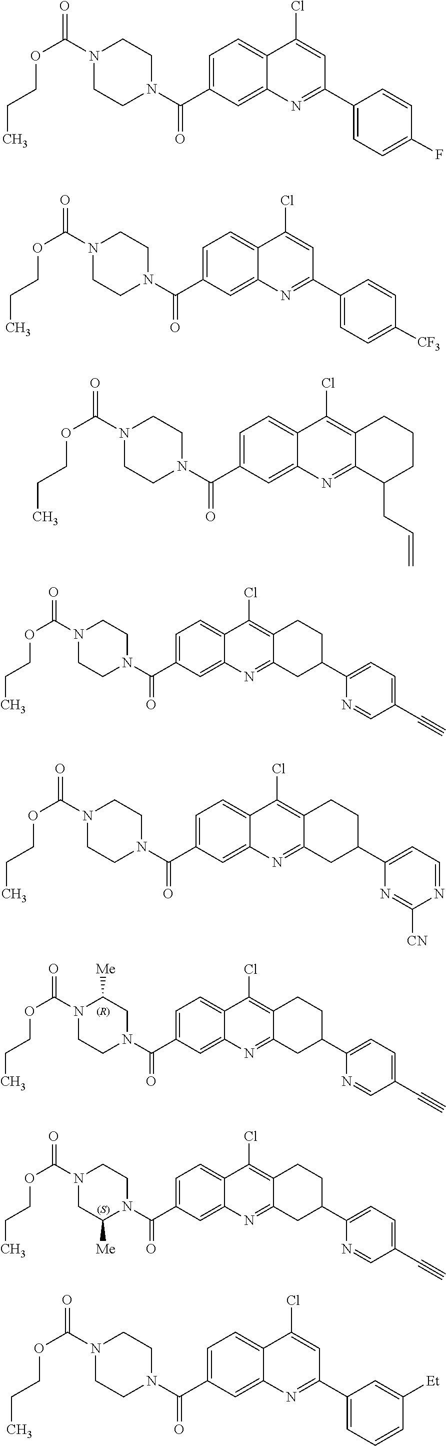

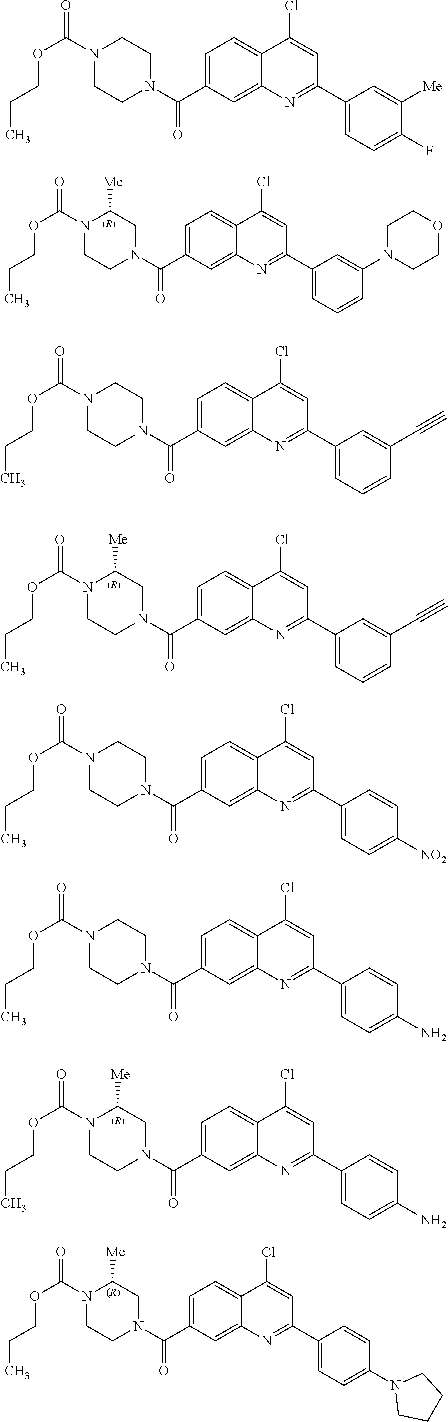

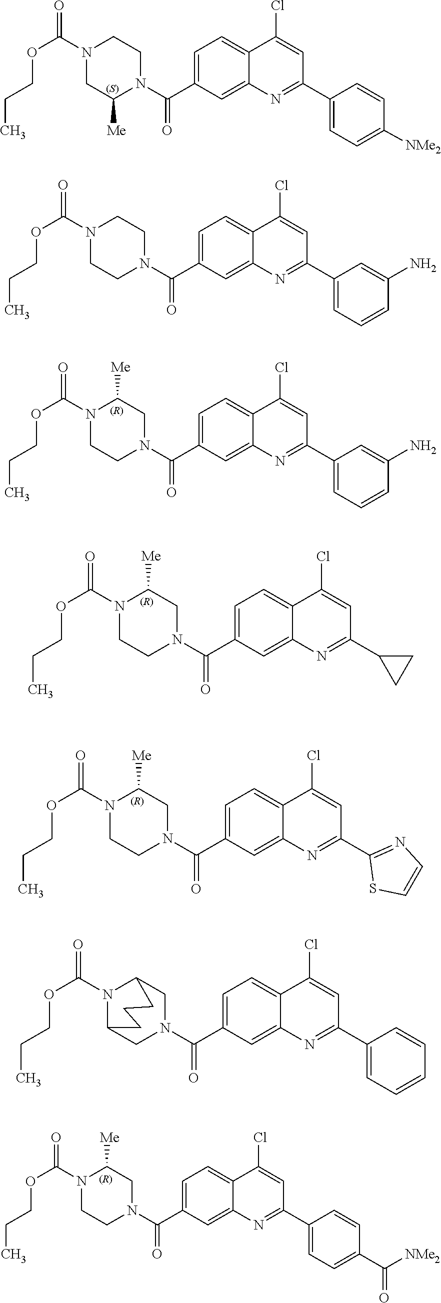

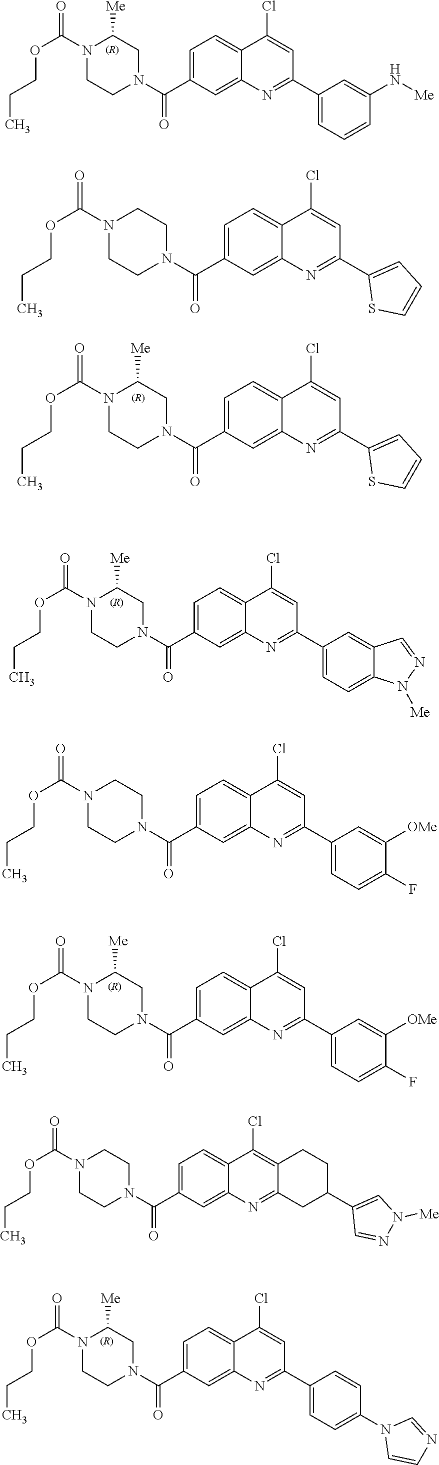

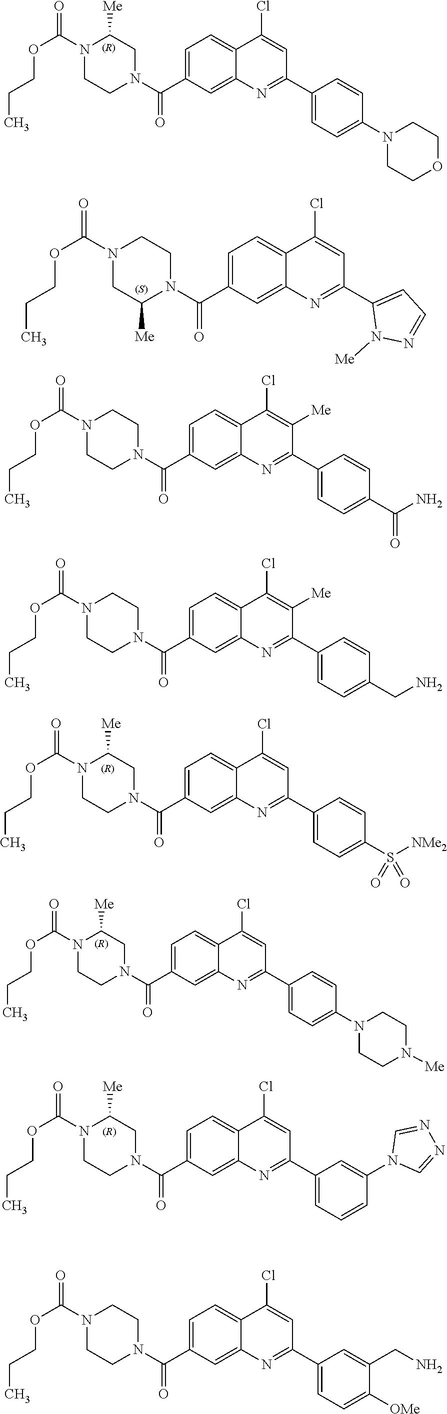

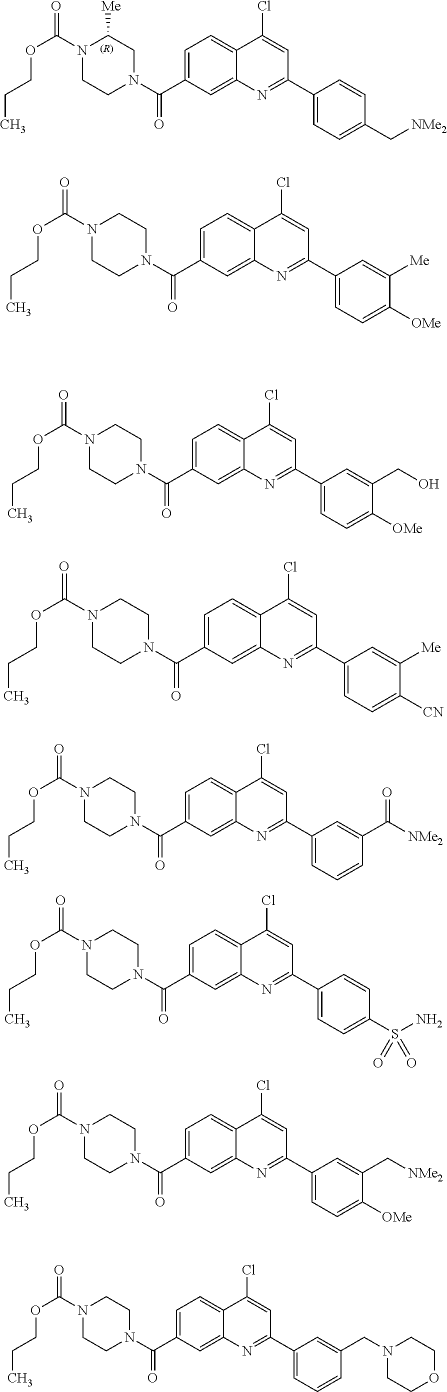

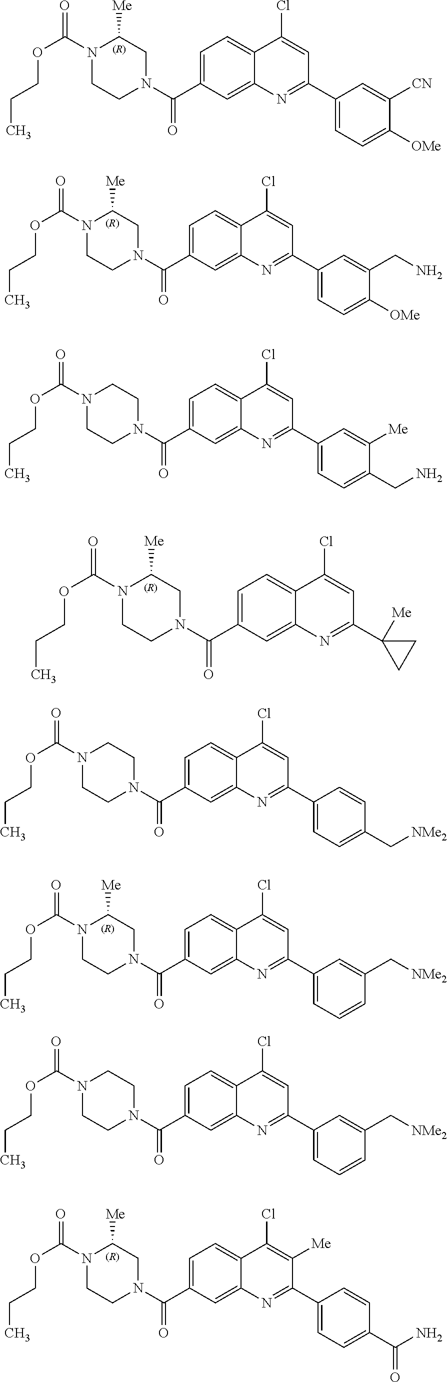

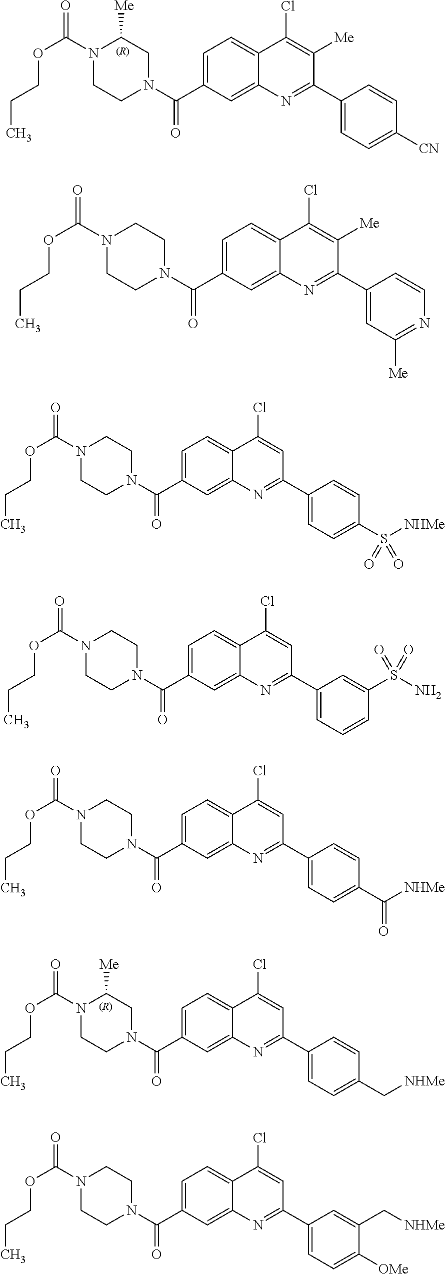

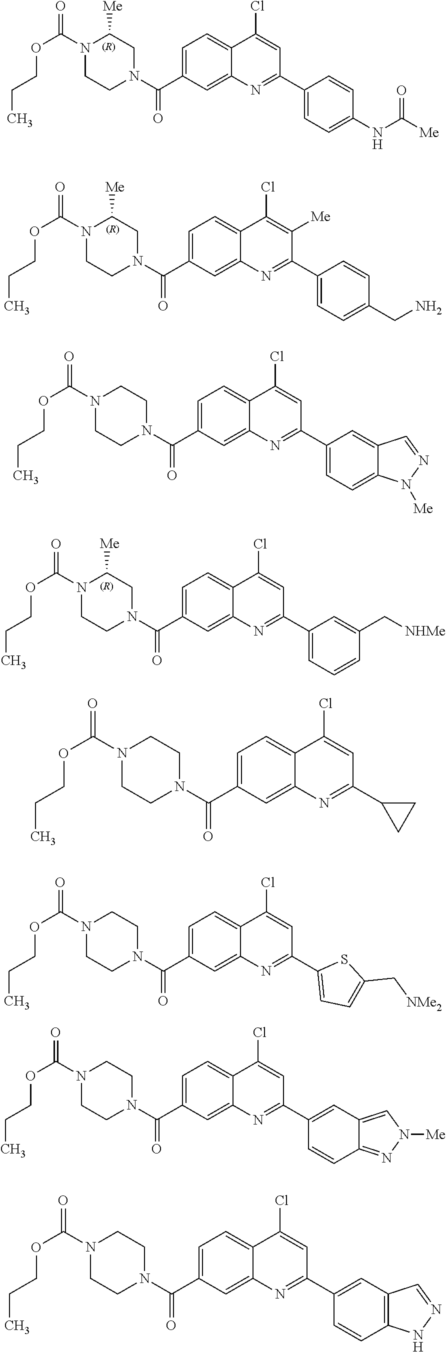

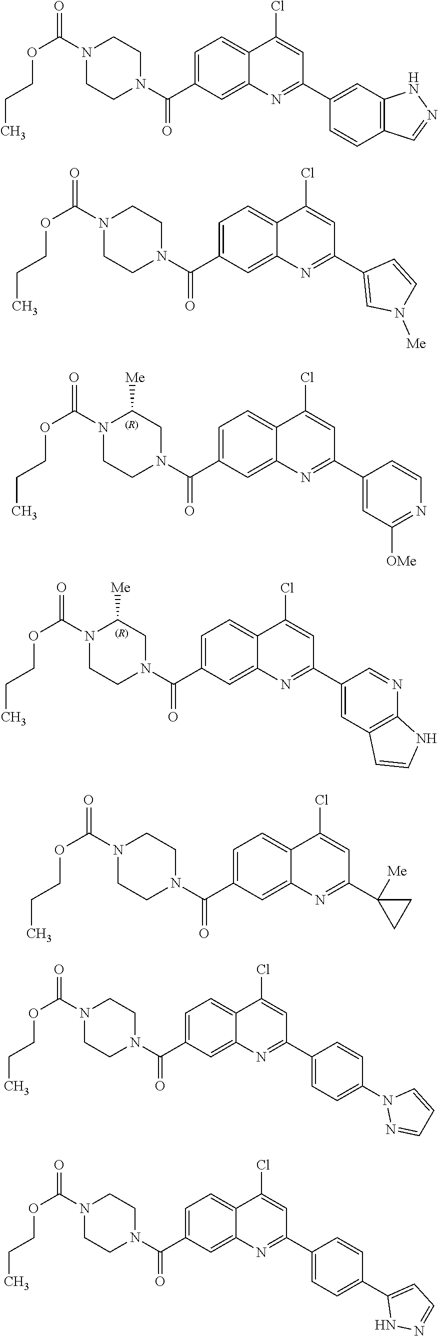

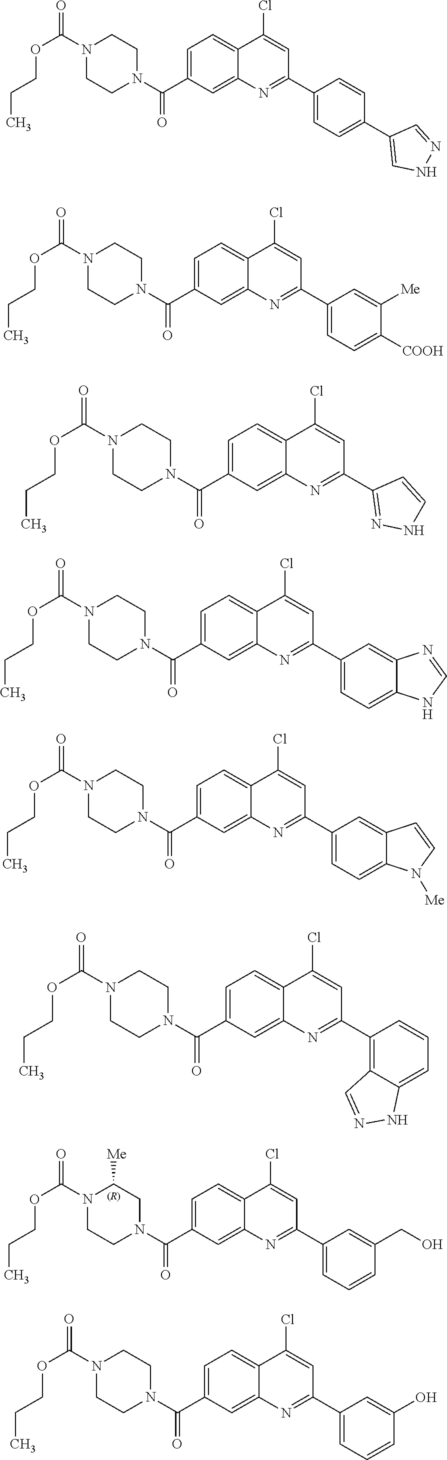

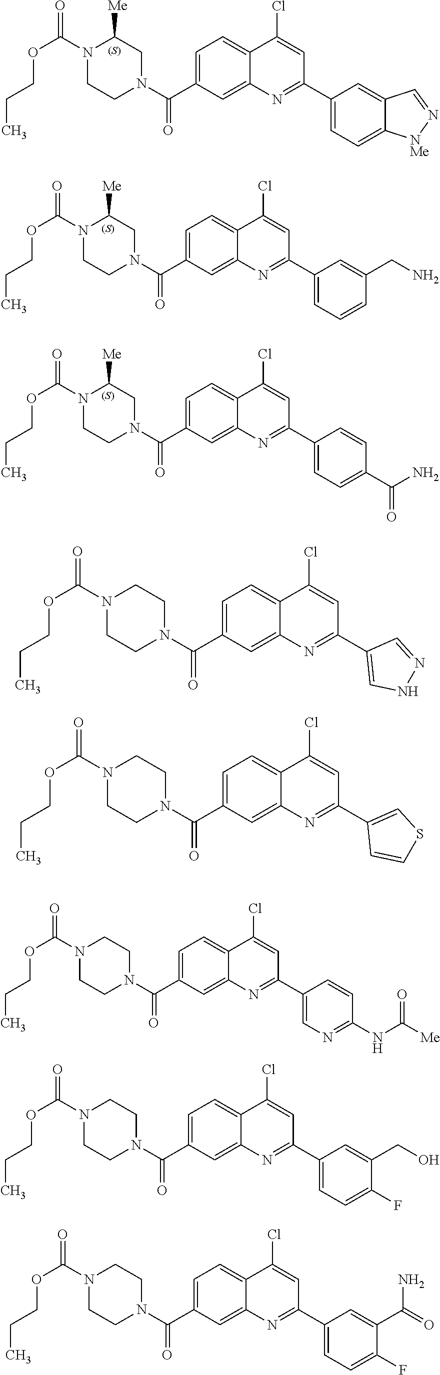

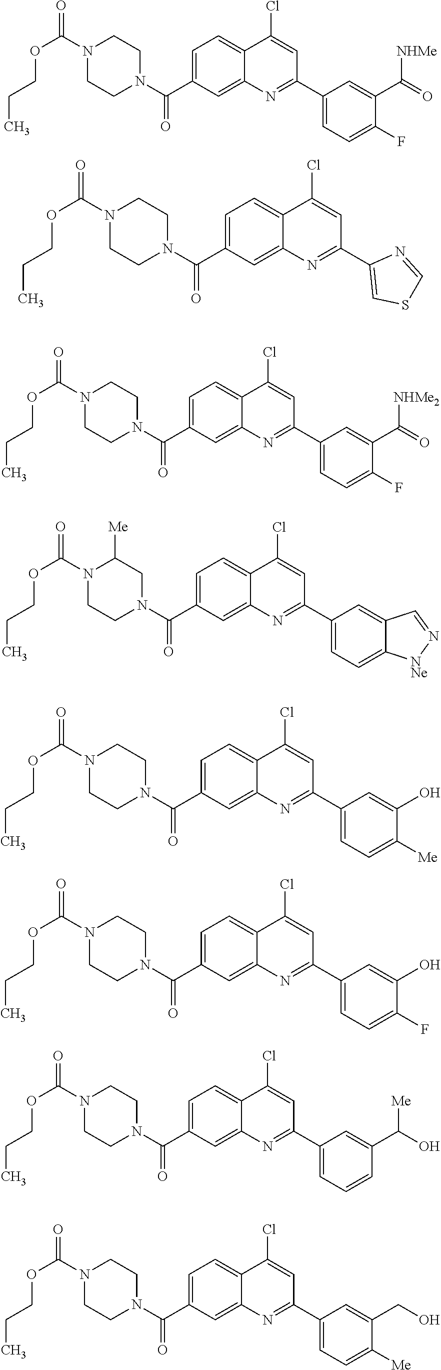

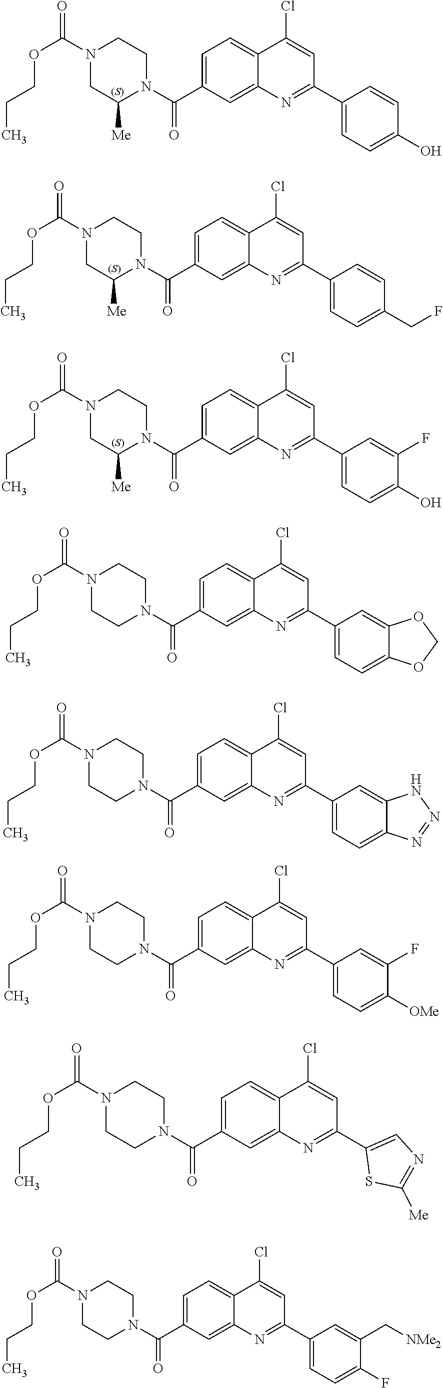

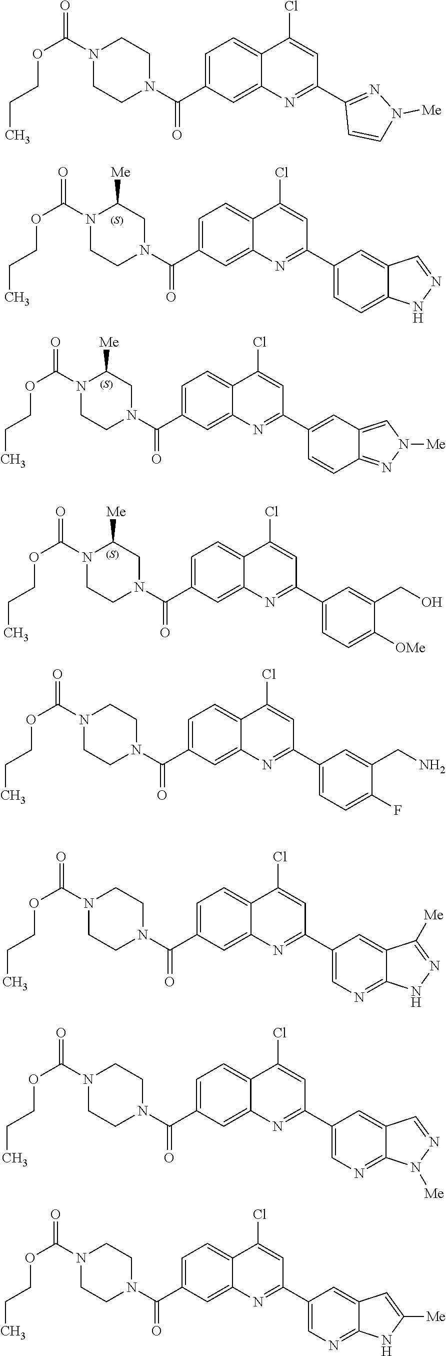

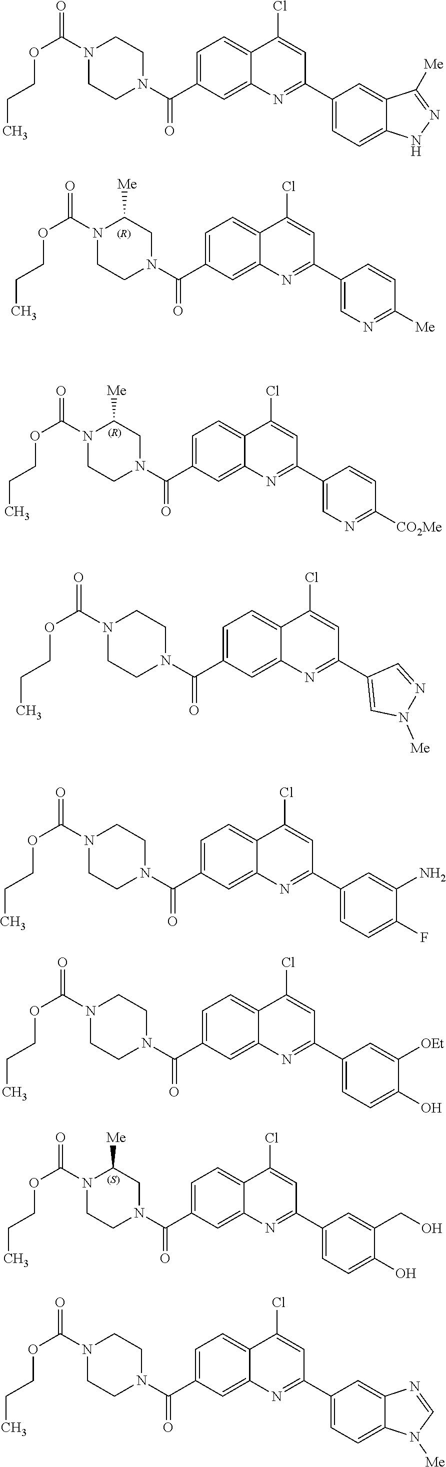

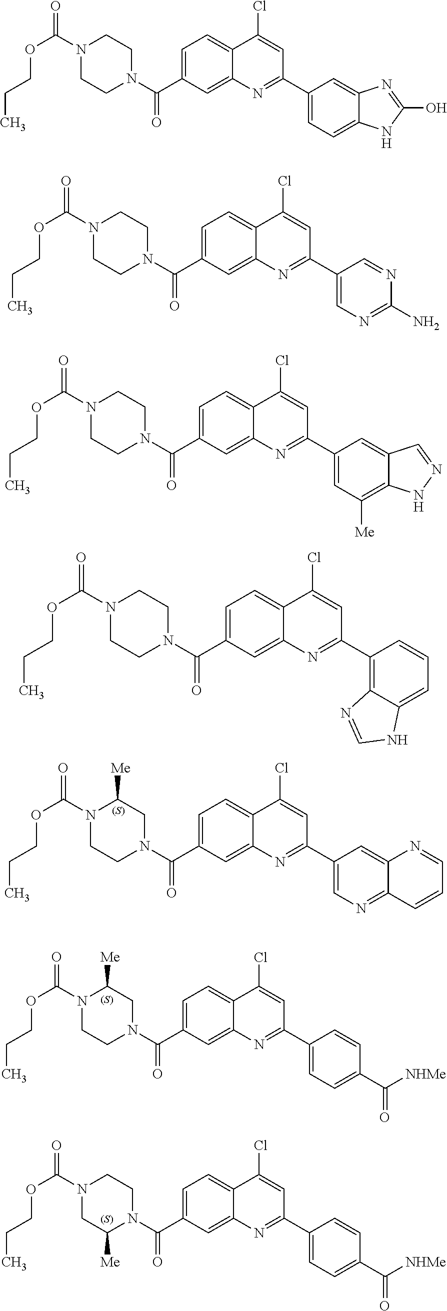

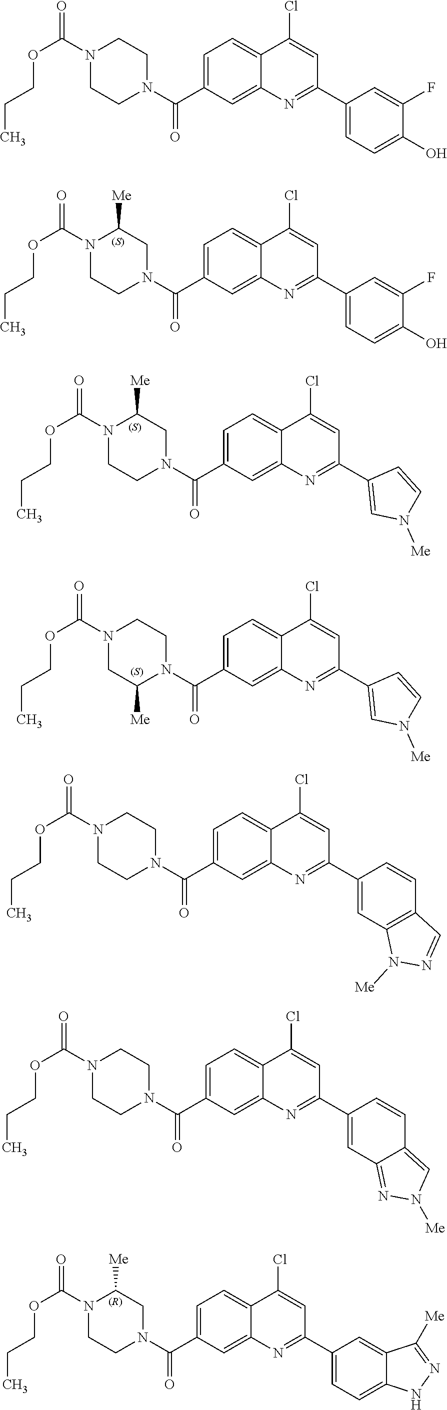

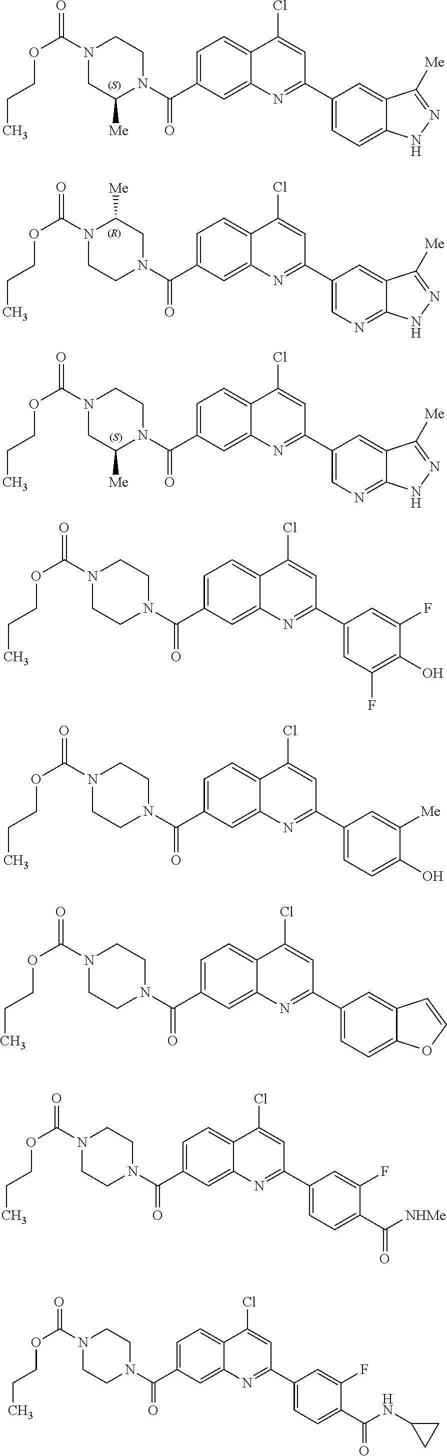

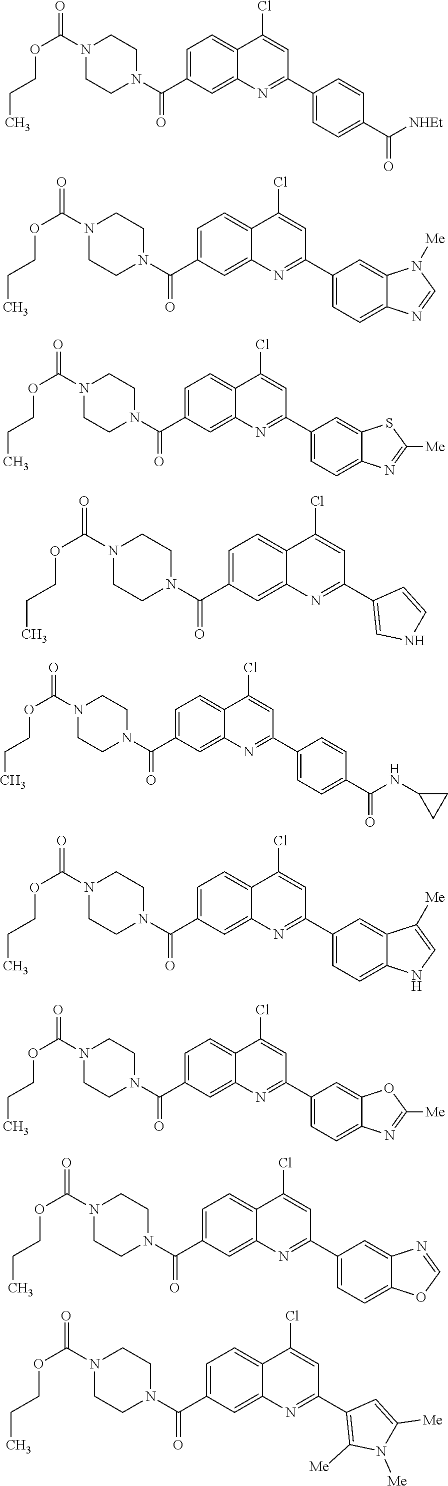

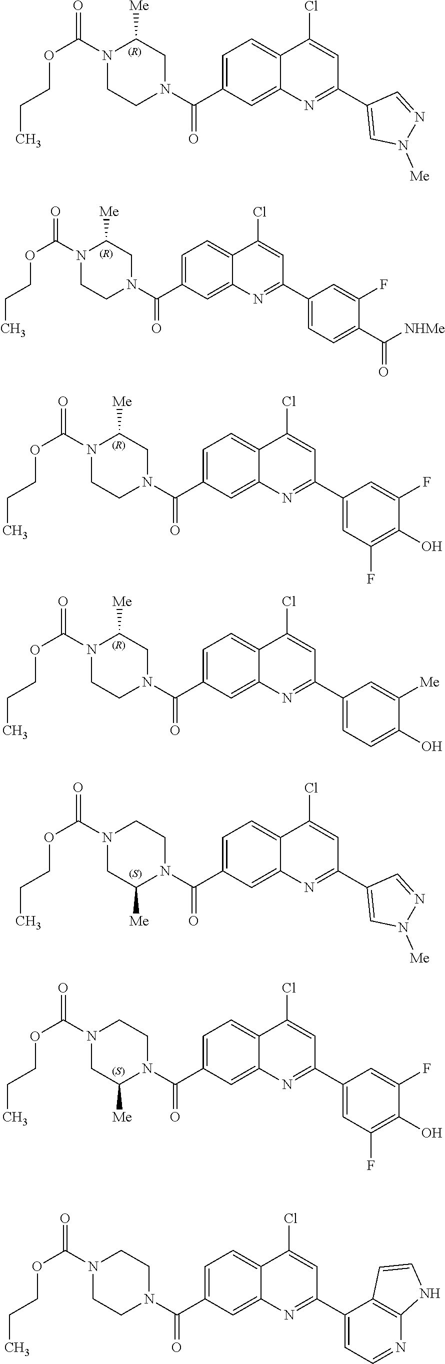

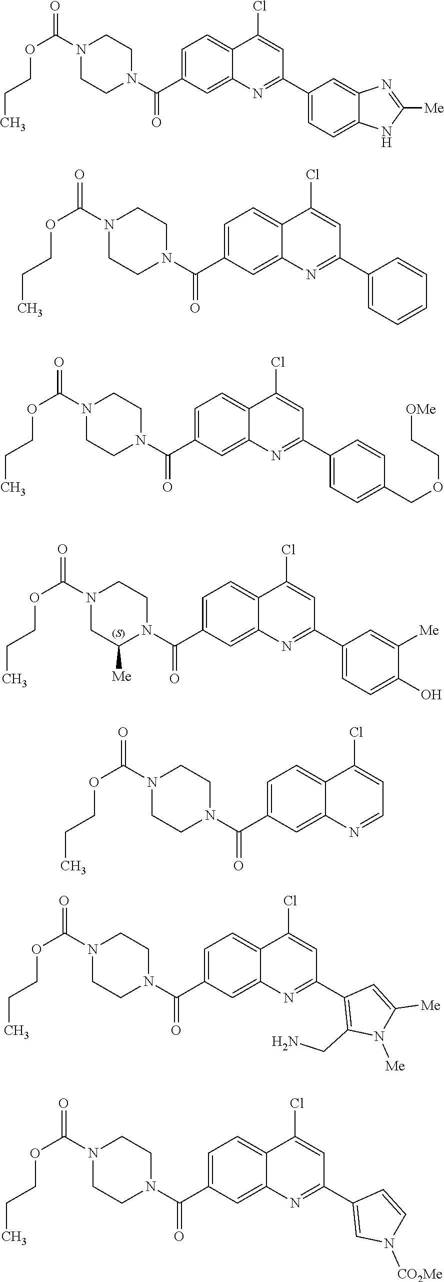

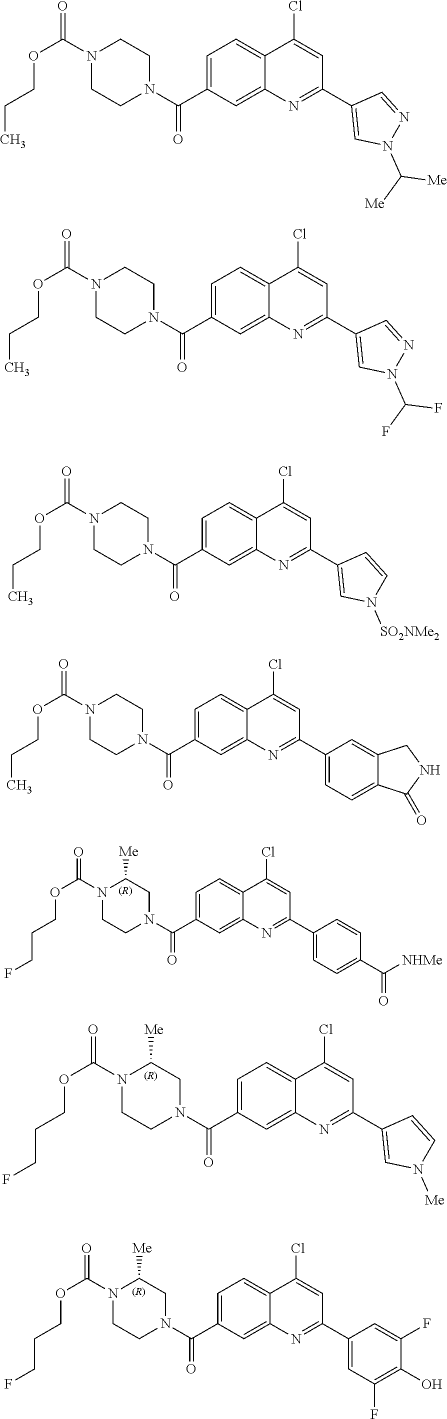

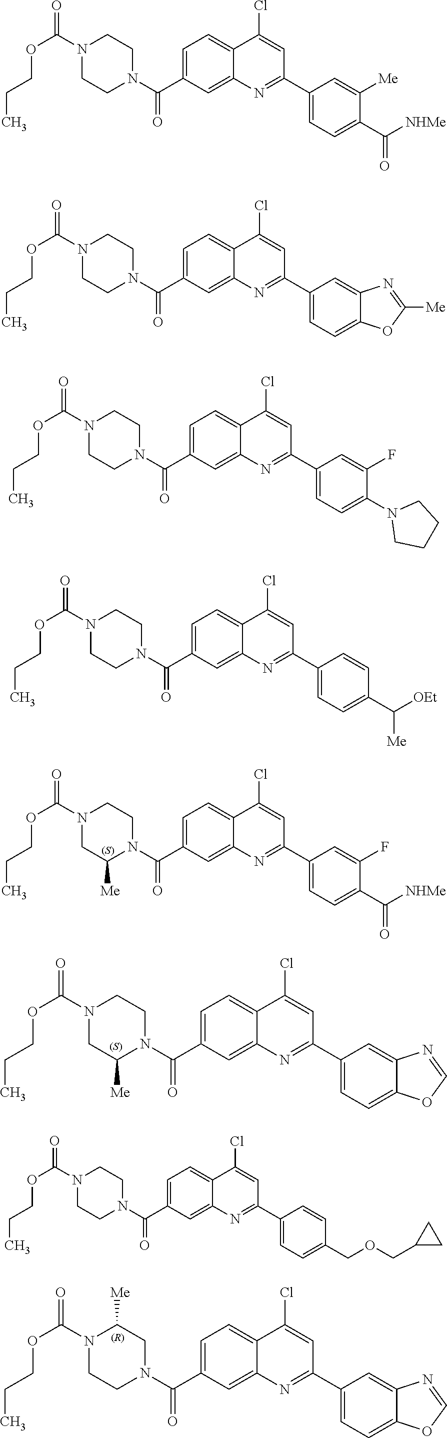

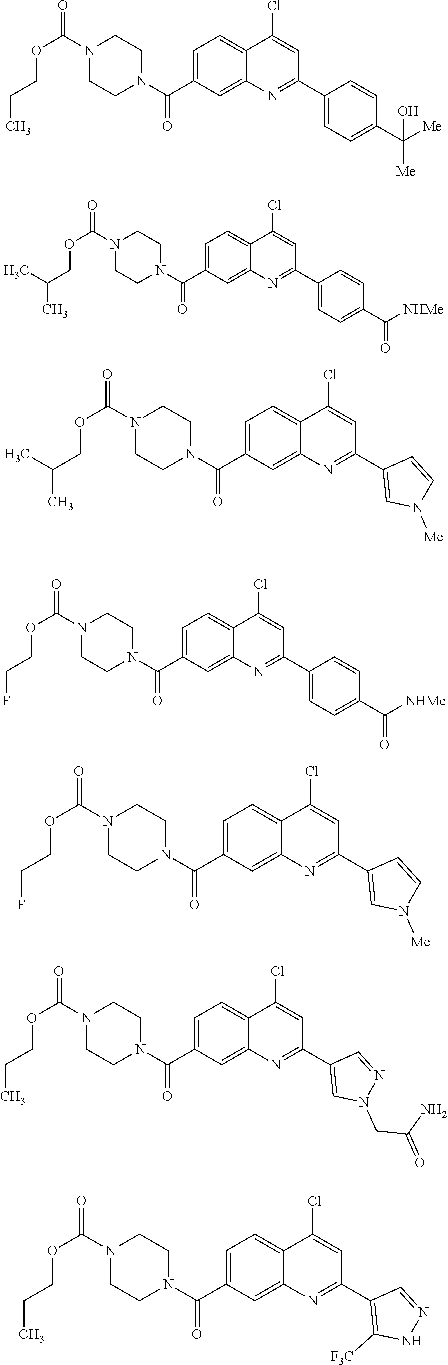

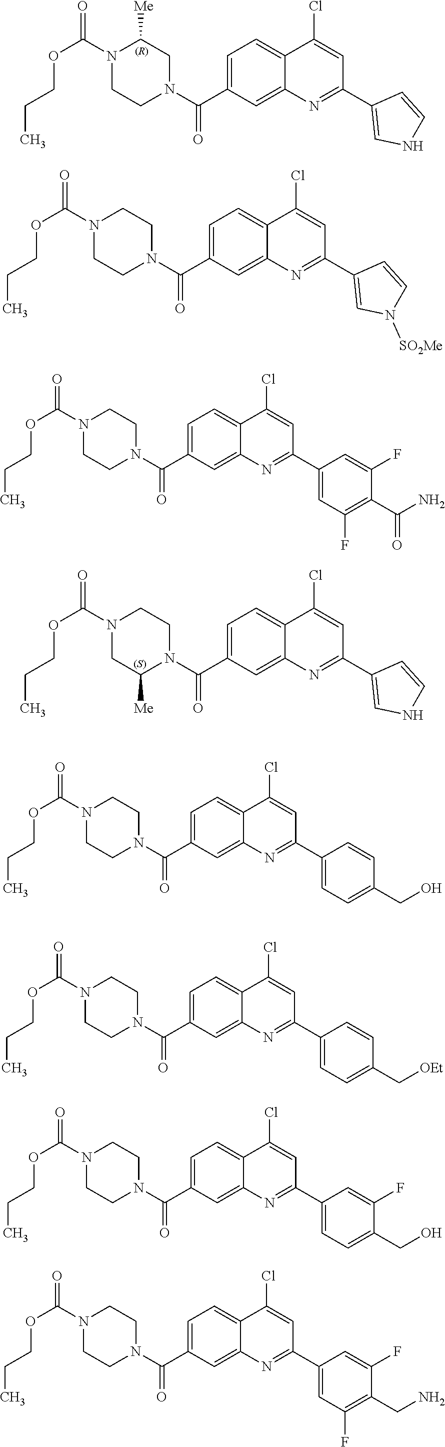

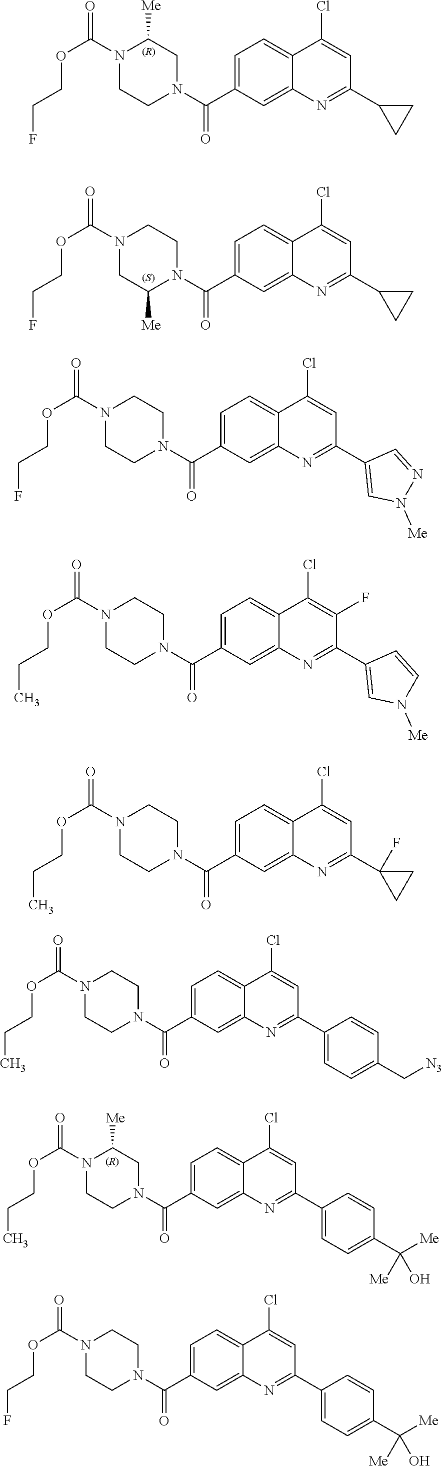

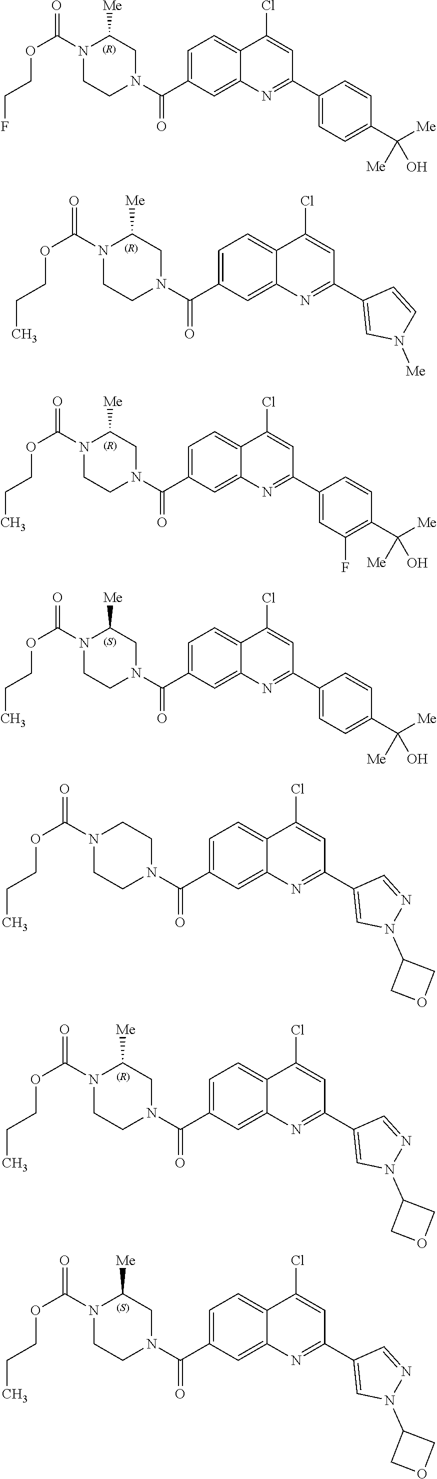

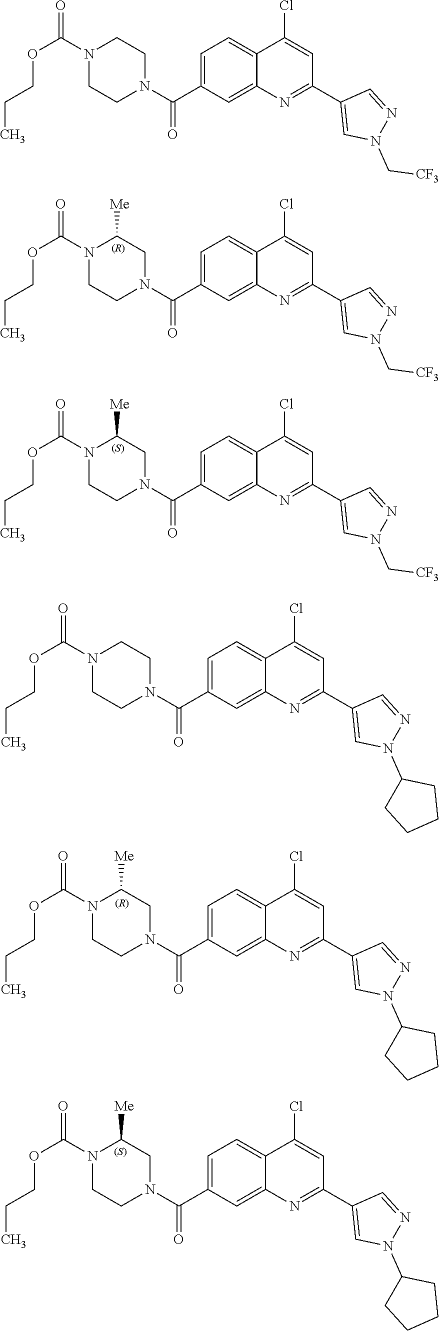

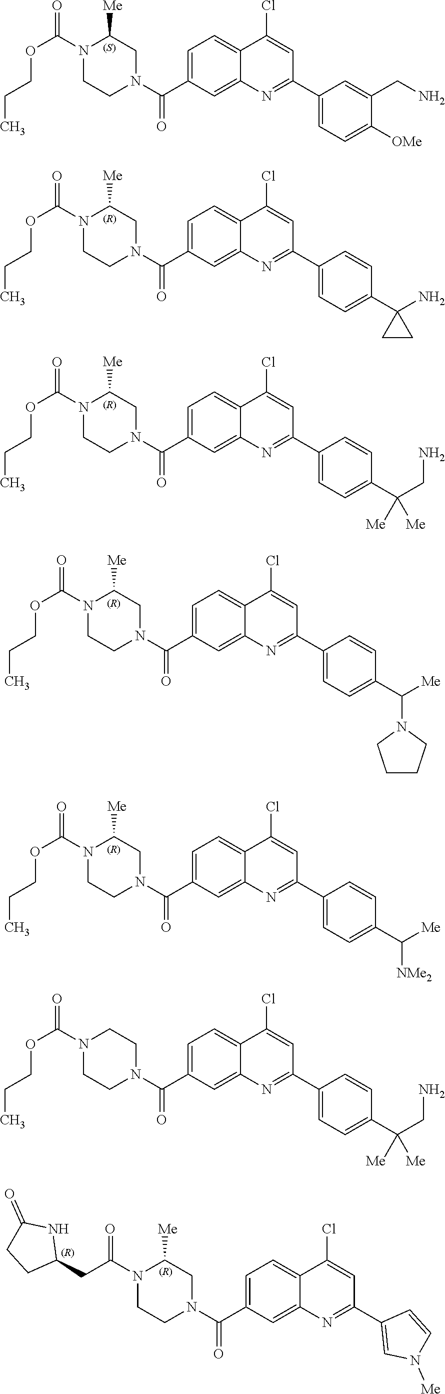

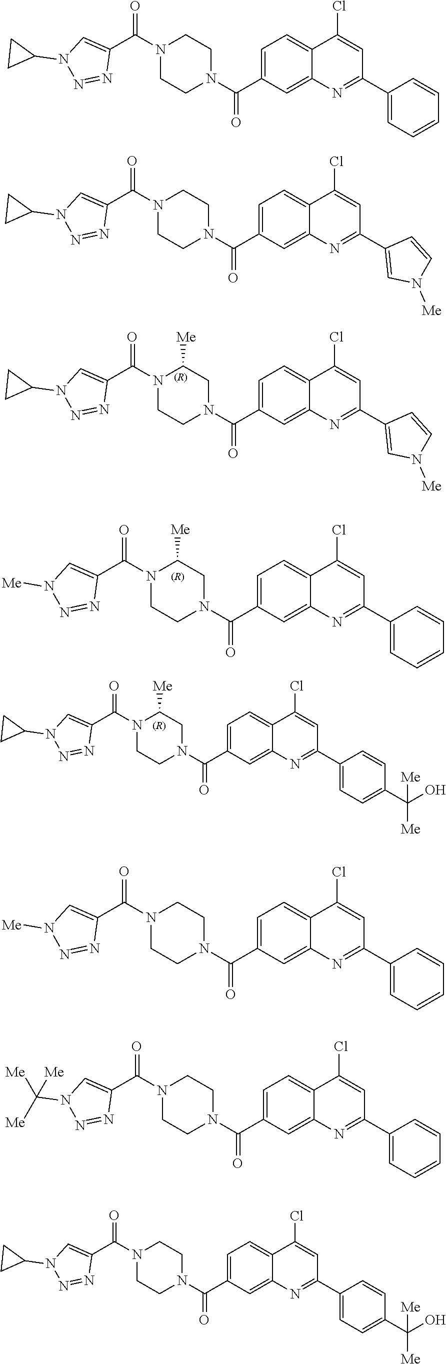

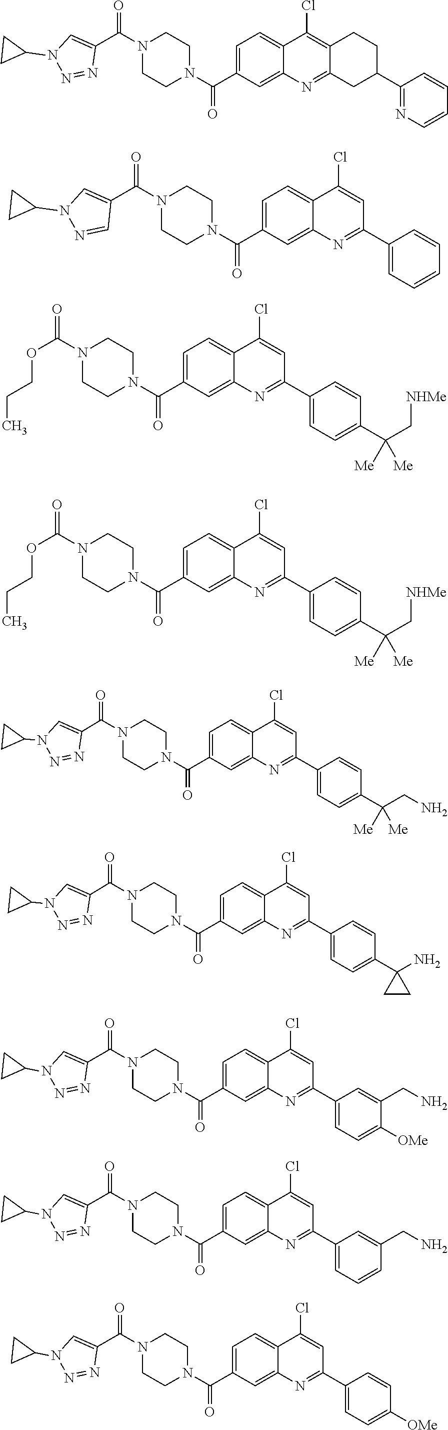

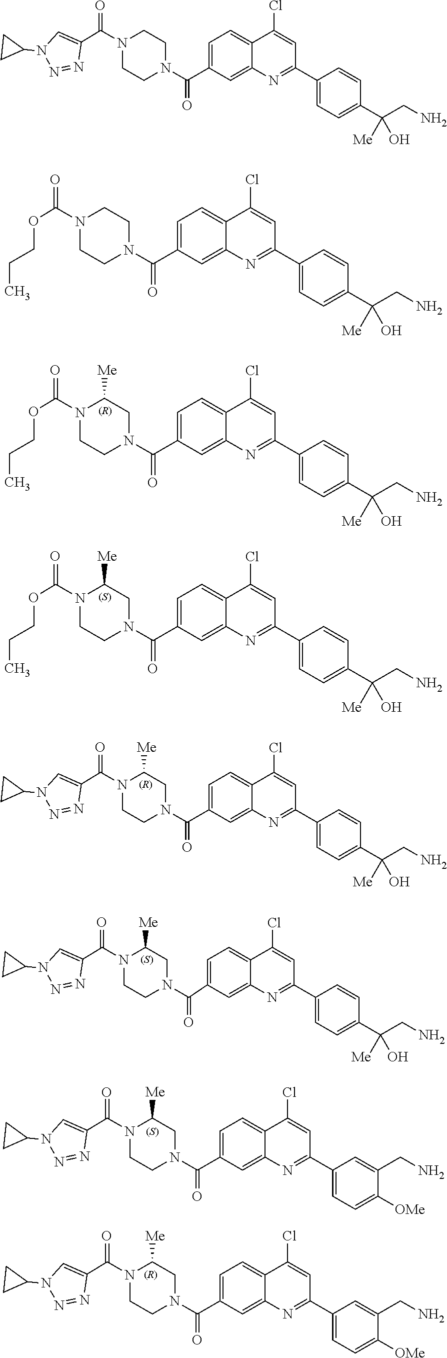

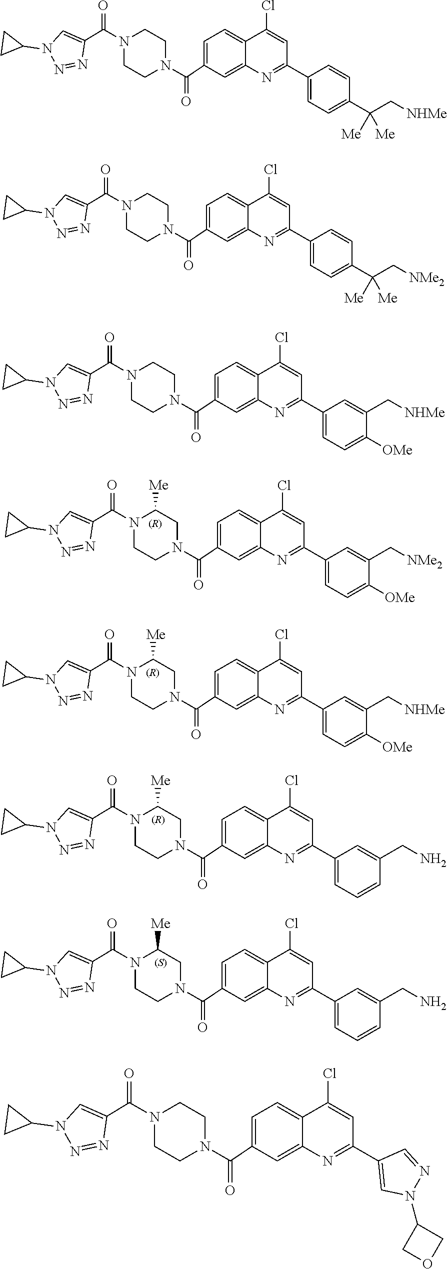

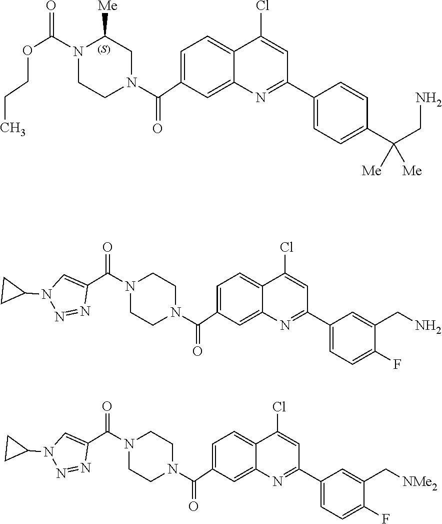

The compound is an enzyme inhibitor. In certain embodiments, the compounds may be a protein lysine methyltransferase (PKMT) inhibitor. It may be an inhibitor for SET domain-containing and non-SET domain-containing methyl transferases. In particular, the protein lysine methyltransferase may be SMYD3.

SMYD3 may also methylate other substrates such as the retinoblastoma (RB1) protein or the vascular endothelial growth factor receptor 1 (VEGFR1) protein.

The compound inhibits methylation of a histone. The histone may be of the H1,H2A, H2B, H3 or H4 family The histone may be of the H1F, H1H1,H2AF, H2A1,H2A2,H2BF, H2B1,H2B2,H3A1, H3A2,H3A3,H41 or H44 subfamily. The histone may be H1F0,H1FNT, H1FOO, H1FX, HIST1H1A, HIST1H1B, HIST1H1C, HIST1H1D, HIST1H1E, HIST1H1T, H2AFB1,H2AFB2,H2AFB3,H2AFJ, H2AFV, H2AFX, H2AFY, H2AFY2,H2AFZ, HIST1H2AA, HIST1H2AB, HIST1H2AC, HIST1H2AD, HIST1H2AE, HIST1H2AG, HIST1H2AI, HIST1H2AJ, HIST1H2AK, HIST1H2AL, HIST1H2AM, HIST2H2AA3,HIST2H2AC, H2BFM, H2BFS, H2BFWT, HIST1H2BA, HIST1H2BB,HIST1H2BC, HIST1H2BD, HIST1H2BE, HIST1H2BF, HIST1H2BG, HIST1H2BH, HIST1H2BI, HIST1H2BJ, HIST1H2BK, HIST1H2BL, HIST1H2BM, HIST1H2BN, HIST1H2BO, HIST2H2BE, HIST1H3A, HIST1H3B, HIST1H3C, HIST1H3D, HIST1H3E, HIST1H3F, HIST1H3G, HIST1H3H, HIST1H3I, HIST1H3J, HIST2H3C, HIST3H3,HIST1H4A, HIST1H4B, HIST1H4C, HIST1H4D, HIST1H4E, HIST1H4F, HIST1H4G, HIST1H4H, HIST1H4I, HIST1H4J, HIST1H4K, HIST1H4L, HIST4H4.

The compound inhibits methylation of histone by inhibiting lysine methyltransferases. The compound may inhibit ASH1L, DOT1L, EHMT1, EHMT2, EZH1, EZH2, MLL, MLL2, MLL3, MLL4, MLL5, NSD1, NSD2, NSD3, PRDM2, PRDM9, SET, SETBP1, SETD1A, SETD1B, SETD2, SETD3, SETD4, SETD5, SETD6, SETD7, SETD8, SETD9, SETDB1, SETDB2, SETMAR, SMYD1, SMYD2, SMYD3, SMYD4, SMYD5, SUV39H1, SUV39H2, SUV420H1, or SUV420H2.

The compound inhibits the trimethylation of histone H3 at lysine 4 (H3K4me3) and/or methylation of histone H4 at lysine 5 (H4K5me).

SMYD3 may regulate multiple overlapping MAP kinase pathway proteins. Accordingly, the compound of the present disclosure may modulate myostatin transcription and/or c-Met transcription.

The compound is assumed to inhibit the MEK-ERK mitogen-activated protein-kinase pathway.

The compound may inhibit methylation of a lysine residue on MAP3K2.

The lysine residue may be K260.

The compound may be administered alone or in the form of a pharmaceutical composition in combination with a pharmaceutically acceptable carrier, diluent or excipient. The compounds, while effective themselves, may be typically formulated and administered in the form of their pharmaceutically acceptable salts as these forms are typically more stable, more easily crystallized and have increased solubility.

The compound may, however, typically be used in the form of pharmaceutical compositions which are formulated depending on the desired mode of administration.

A pharmaceutical composition may comprise a compound as disclosed above, or a pharmaceutically acceptable form or prodrug thereof, and a pharmaceutically acceptable excipient. The compositions may be prepared in manners well known in the art.

The amount of compound in the compositions may be such that it is effective to measurably inhibit one or both of methylation of histone H3 at lysine 4 (H3K4me3) and of histone H4 at lysine 5 (H4K5me) in a biological sample or in a patient. The composition may be formulated for administration to a patient in need of such composition.

In using the compounds, they may be administered in any form or mode which may make the compound bioavailable. One skilled in the art of preparing formulations can readily select the proper form and mode of administration depending upon the particular characteristics of the compound selected, the condition to be treated, the stage of the condition to be treated and other relevant circumstances.

The term “pharmaceutically acceptable excipient” may refer to a non-toxic carrier, adjuvant, or vehicle that does not destroy the pharmacological activity of the compound with which it is formulated. Pharmaceutically acceptable carriers, adjuvants or vehicles that may be used in the compositions of this disclosure may include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol or wool fat.

Compositions as defined above may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions as defined above may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Pharmaceutical compositions for parenteral injection may comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of micro-organisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminium monostearate and gelatin.

If desired, and for more effective distribution, the compounds may be incorporated into slow release or targeted delivery systems such as polymer matrices, liposomes, and microspheres.

The injectable formulations may be sterilized, for example, by filtration through a bacterial-retaining filter, or by incorporating sterilizing agents in the form of sterile solid compositions that can be dissolved or dispersed in sterile water or other sterile injectable medium just prior to use.

Alternatively, pharmaceutically acceptable compositions as defined above may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions as defined above may also be administered topically, especially when the target of treatment includes areas or organs readily accessible by topical application, including diseases of the eye, the skin, or the lower intestinal tract. Suitable topical formulations may be readily prepared for each of these areas or organs.

Topical application for the lower intestinal tract may be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of compounds as defined above may include, but are not limited to, mineral oil, liquid petrolatum, white petrolatum, propylene glycol, polyoxyethylene, polyoxypropylene compound, emulsifying wax and water. Alternatively, the pharmaceutically acceptable compositions may be formulated in a suitable lotion or cream containing the active components suspended or dissolved in one or more pharmaceutically acceptable carriers. Suitable carriers may include, but are not limited to, mineral oil, sorbitan monostearate, polysorbate 60, cetyl esters wax, cetearyl alcohol, 2 octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions as defined above may also be administered by nasal aerosol or inhalation. Such compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions as defined above may be formulated for oral administration. Such formulations may be administered with or without food. In some embodiments, pharmaceutically acceptable compositions as defined above may be administered without food. In other embodiments, pharmaceutically acceptable compositions as defined above may be administered with food.

The amount of compound that may be combined with the carrier materials to produce a composition in a single dosage form may vary depending upon the host treated, the particular mode of administration. Preferably, the compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

A method of inhibiting SMYD3 in a cell may comprise administering to a cell a compound as disclosed above, or a pharmaceutically acceptable form or prodrug thereof, or a composition as disclosed above.

The activity of a compound as an inhibitor may be assayed in vitro, in vivo or in a cell line. In vitro assays may include assays that determine inhibition of either the methylation activity and/or the subsequent functional consequences, or methylation activity of one or both of histone H3 at lysine 4 (H3K4me3) and histone H4 at lysine 5 (H4K5me), or the methylation of a lysine residue on MAP3K2. In the in vitro assay, SMYD3 catalyzes the methylation of the MAP3K2 peptide substrate by transferring a methyl group from SAM to MAP3K2 peptide and further converts the SAM to SAH. The SMYD3 methyltransferase activity is measured based on the amount of SAH produced from the reaction through the use of coupling enzymes that convert the SAH to ATP.

The inhibition of SMYD3 further comprises the inhibition of cell proliferation.

The cell may be in vitro.

The cell may be from a cell line.

The cell line may be an immortalized cell line, a genetically modified cell line or a primary cell line.

The cell line may be selected from the group consisting of HepG2,HCT116, A549,HPAF-II, CFPAC-1,HuH7, SNU398,Hep3B, PLC/PRF/5,HuH1, Bel7404,HCCLM3,HLE, SK-HEP-1, Mahlavu, JHH1, JHH2, JHH4, JHH5, JHH7, SNU354, SNU368, SNU387, SNU423, SNU449, SNU739, SNU761, SNU886, MIA PaCa-2 and HEK293.

The cell may be from tissue of a subject.

The cell may be in a subject.

A method of treating a SMYD-3-related disorder may comprise administering to a subject in need of treatment a compound as disclosed above, or a pharmaceutically acceptable form or prodrug thereof, or a composition as disclosed above.

The method as disclosed above may further comprise the step of administering an additional therapeutic agent in the subject.

The compound as disclosed above, or a pharmaceutically form or prodrug thereof, or a composition as disclosed above may be for use in therapy.

The use of a compound as disclosed above, or a pharmaceutically acceptable form or prodrug thereof, or a composition as disclosed above, may be in the manufacture of a medicament for treatment of a SMYD3-related disorder.

The medicament may be administered with an additional therapeutic agent, wherein said medicament may be administered in combination or alteration with the additional therapeutic agent.

A compound as disclosed above, or a pharmaceutically acceptable form or prodrug thereof, or a composition as disclosed above, may be for use in the treatment of a SMYD3-related disorder.

The disorder may be cancer, angiogenic disorder or pathological angiogenesis, fibrosis or inflammatory conditions.

The disorder may be lymphoma, cutaneous T-cell lymphoma, follicular lymphoma, or Hodgkin lymphoma, cervical cancer, ovarian cancer, breast cancer, lung cancer, prostate cancer, colorectal cancer, gastric cancer, pancreatic cancer, sarcoma, hepatocellular carcinoma, leukemia or myeloma, retinal angiogenic disease, liver fibrosis, kidney fibrosis, or myelofibrosis.

The compound may be administered with an additional therapeutic agent, wherein said medicament may be administered in combination or alteration with the additional therapeutic agent.

A process for synthesizing the compound as disclosed above, having the following Formula (III), may comprise the steps of:

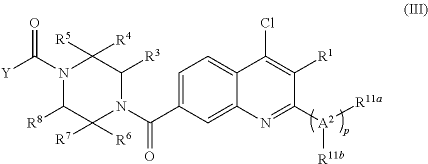

(a) contacting an optionally substituted aminobenzoate ester with a compound having the following Formula (Va) to form a cyclized product;

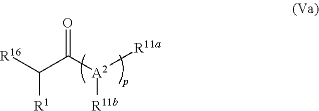

wherein R¹⁶ is selected from the group consisting of H, methyl, COOMe and COOEt;

(b) selectively displacing at least one ketone of the cyclized product of step (a) with a halogen;

(c) selectively hydrolyzing the ester of the cyclized product of step (a) to a carboxylic acid and selectively functionalizing the carboxylic acid with a group having the following formula (VI) under reaction conditions to form the compound of formula (III);

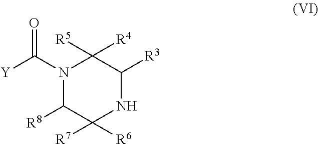

wherein step (b) and (c) may be performed simultaneously, sequentially or in any order.

By way of illustration, the compounds, esters, amides, salts and solvates of Formula (III) may be prepared by a process which comprises an initial reaction step (a) between an aminobenzoate and a carbonyl-containing moiety of Formula (Va). This reaction may be carried out in a solvent. It may occur in a high-boiling solvent. The solvent may be selected from the group consisting of toluene, 1,4-dioxane, n-butanol, diphenyl ether, chlorobenzene, carbon tetrachloride, diethylene glycol, diglyme, hexamethylphosphoramide, o-xylene, m-xylene and p-xylene. The reaction temperature may be in a range of about 100 to about 400° C., or about 150 to about 400° C., or about 200 to about 400° C., or about 250 to about 400° C., or about 300 to about 400° C., or about 350 to about 400° C., or about 150 to about 350° C., or about 150 to about 300° C., or about 150 to about 250° C., or about 150 to about 200° C., or about 150 to about 350° C., or about 200 to about 300° C., or about 250 to about 300° C., e.g. at about 100° C., at about 150° C., at about 200° C., at about 250° C., at about 300° C., at about 350° C., or at about 400° C. It may be heated in a sealed tube. It may be in a reflux apparatus. It may be heated by an oil bath or a sand bath. It may be heated using microwave irradiation. The reaction time may vary between 30 min to 6 hours. It may vary in a range of about 30 min to 6 hours, or about 1 hour to 6 hours, or about 1.5 hours to 6 hours, or about 2 hours to 6 hours, or about 2.5 hours to 6 hours, or about 3 hours to 6 hours, or about 3.5 hours to 6 hours, or about 4 hours to 6 hours, or about 5 hours to 6 hours, or about 30 min to 5 hours, or about 30 min to 4 hours, or about 30 min to 3 hours, or about 30 min to 2 hours, or about 30 min to 1 hour, e.g. it may be about 30 min, or about 1 hour, or about 2 hours, or about 3 hours, or about 4 hours, or about 5 hours, or about 6 hours. After the reaction is complete, that reaction solution may be diluted with a non-polar solvent. The non-polar solvent may be selected from pentane, hexane, heptane, methyl t-butyl ether, petroleum ether and dichloromethane. The product may precipitate out of the solution.

In reaction step (b) the ensuing amino-enone may be treated with a halogenating reagent. The halogenating reagent may be phosphoryl-containing. It may be phosphorous oxychloride. Alternatively, it may be oxalyl chloride. The reaction may be in a solvent. It may be in a non-polar solvent. It may be in a solvent selected from the group consisting of hexane, cyclohexane, benzene, toluene, 1,4-dioxane, chloroform, diethyl ether and dichloromethane. It may be at an elevated temperature. The temperature may be in a range of about 30 to 120° C., or about 50 to 120° C., or about 70 to 120° C., or about 90 to 120° C., or about 110 to 120° C., or about 30 to 100° C. or about 30 to 80° C., or about 30 to 60° C., or about 30 to 40° C. or at about 30° C., or at about 50° C., or at about 70° C., or at about 90° C., or at about 110° C., or at about 130° C. The reaction time may be between about 30 min and 4 hours, or between about 1 hour and 4 hours, or between about 2 hours and 4 hours, or between about 3 hours and 4 hours, or between about 30 min and 3 hours, or between about 30 min and 2 hours, or between about 30 min and 1 hour, or about 30 min, or about 1 hour, or about 2 hours, or about 3 hours, or about 4 hours. The reaction product may be purified by aqueous work-up followed by chromatography.

For the hydrolysis of reaction step (c), the ester-containing starting material may be treated with a base in a solvent. The base may be selected from a variety of bases including inorganic bases or nitrogen bases. For example, triethylamine, pyridine, sodium hydroxide, potassium hydroxide, lithium hydroxide or sodium bicarbonate may be used. For example, the base may be lithium hydroxide. The solvent mixture may contain two solvents. At least one of these solvents may be a polar solvent. The solvent mixture may contain methanol. The solvent mixture may contain 1,4-dioxane. The solvent mixture may be a mixture, for example, of methanol and 1,4-dioxane. The reaction may be followed by an aqueous work-up under acidic conditions. The pH of the aqueous work-up may be adjusted to about 2, about 3, about 4, or about 5, it may be, for example, 3.

The selective functionalizing of the carboxylic acid of step (c) may be performed under peptide coupling reaction conditions known to the person skilled in the art. In particular, it may involve a peptide coupling reagent selected from the group consisting of HATU, N,N′-dicyclohexylcarbodiimide, HBTU, hydroxybenzotriazole, propyl phosphonic anhydride and phosphorous oxychloride. It may involve a base selected from the group of nitrogen bases. It may involve bases selected from the group of inorganic bases. For example, triethylamine, pyridine, sodium hydroxide, potassium hydroxide or sodium bicarbonate may be used. A solvent may be used. The solvent may include polar aprotic solvents. The solvent may be selected from the group consisting of tetrahydrofuran, ethyl acetate, acetone, DMF, acetonitrile, dimethylsulfoxide or nitromethane. The reaction may be performed at room temperature. The reaction time may be between about 1 hour and 16 hours, or between about 1 hour and 14 hours, or between about 1 hour and 12 hours, or between about 1 hour and 10 hours, or between about 1 hour and 8 hours, or between about 1 hour and 6 hours, between about 1 hour and 4 hours, between about 1 hour and 2 hours, between about 3 hours and 16 hours, between about 5 hours and 16 hours, between about 6 hours and 16 hours, between about 8 hours and 16 hours, between about 10 hours and 16 hours, between about 12 hours and 16 hours, between about 14 hours and 16 hours, or about 1 hour, or about 2 hours, or about 4 hours, or about 6 hours, or about 8 hours, or about 10 hours, or about 12 hours, or about 14 hours, or about 16 hours. The reaction product may be purified by aqueous work-up followed by chromatography.

A process for synthesizing the compound as disclosed above, having the following Formula (III), wherein R¹ is optionally a halogen or hydrogen, may comprise the steps of:

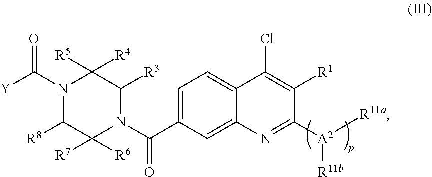

(a) contacting an optionally substituted aminobenzoate ester with a compound having the following Formula (Vb) and phosphorus oxychloride to form a halogenated cyclized product;

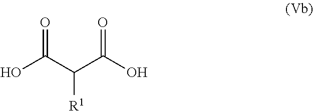

(b) selectively hydrolyzing the ester of the cyclized product of step (a) to a carboxylic acid and selectively functionalizing the carboxylic acid with a group having the following formula (VI) under reaction conditions to form an amide; and

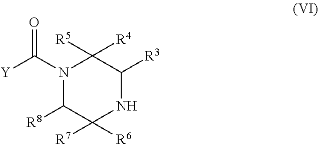

(c) selectively functionalizing at least one halogen of the halogenated cyclized product of step (a) with a group having the following formula (VIIa) or formula (VIIb) under reaction conditions to form the compound of formula (III);

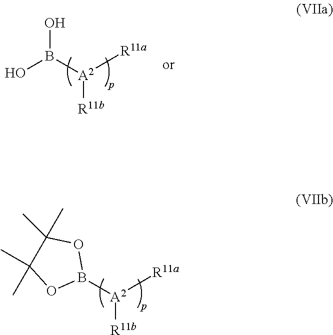

wherein step (b) and (c) may be performed simultaneously, sequentially or in any order.

By way of illustration, the compounds, esters, amides, salts and solvates of Formula (III) may alternatively be prepared by a process which comprises an initial reaction step (a) between an aminobenzoate and a carbonyl-containing moiety of Formula (Vb). The starting materials may be treated with a halogenating reagent. The halogenating reagent may be phosphoryl-containing. It may be phosphorous oxychloride. Alternatively, it may be oxalyl chloride. The reaction may occur without a solvent. The temperature for mixing the solvents may be in a range of about −30 to 10° C., or about −20 to 10° C., or about 10 to 10° C., or about 0 to 10° C., or about −30 to 0° C., or about −30 to −10° C. or about −30 to −20° C., or at about −30° C., or at about −20° C., or at about −10° C., or at about 0° C., or at about 10° C. The reaction temperature may be raised to about 60 to 150° C., or to about 80 to 150° C., or to about 100 to 150° C., or to about 120 to 150° C., or to about 140 to 150° C., or to about 60 to 130° C., or to about 60 to 110° C., or to about 60 to 90° C., or to about 60 to 70° C., or to about 60° C., or to about 80° C., or to about 100° C., or to about 110° C., or to about 130° C., or to about 150° C. The reaction time may be between about 30 min and 4 hours, or between about 1 hour and 4 hours, or between about 2 hours and 4 hours, or between about 3 hours and 4 hours, or between about 30 min and 3 hours, or between about 30 min and 2 hours, or between about 30 min and 1 hour, or about 30 min, or about 1 hour, or about 2 hours, or about 3 hours, or about 4 hours. The reaction product may be purified by aqueous work-up followed by chromatography.

In the hydrolysis of reaction step (b) the ester-containing starting material may be treated with a base in a solvent. The base may be selected from a variety of bases including inorganic bases or nitrogen bases. For example, triethylamine, pyridine, sodium hydroxide, potassium hydroxide, lithium hydroxide or sodium bicarbonate may be used. For example, the base may be lithium hydroxide. The solvent mixture may contain of two solvents. At least one of these solvents may be a polar solvent. The solvent mixture may contain methanol. The solvent mixture may contain 1,4-dioxane. The solvent mixture may be a mixture, for example, of methanol and 1,4-dioxane. The reaction may be followed by an aqueous work-up under acidic conditions. The pH of the aqueous work-up may be adjusted to about 2, about 3, about 4, or about 5, it may be, for example, 3.

The selective functionalizing of the carboxylic acid in step (b) may be performed under peptide coupling reaction conditions known to the person skilled in the art. In particular, it may involve a peptide coupling reagent selected from the group consisting of HATU, N,N′-dicyclohexylcarbodiimide, HBTU, hydroxybenzotriazole, propyl phosphonic anhydride and phosphorous oxychloride. It may involve a base selected from the group of nitrogen bases. It may involve bases selected from the group of inorganic bases. For example, triethylamine, pyridine, sodium hydroxide, potassium hydroxide or sodium bicarbonate may be used. A solvent may be used. The solvent may include polar aprotic solvents. The solvent may be selected from the group consisting of tetrahydrofuran, ethyl acetate, acetone, DMF, acetonitrile, dimethylsulfoxide or nitromethane. The reaction may be performed at room temperature. The reaction time may be between about 1 hour and 16 hours, or between about 1 hour and 14 hours, or between about 1 hour and 12 hours, or between about 1 hour and 10 hours, or between about 1 hour and 8 hours, or between about 1 hour and 6 hours, between about 1 hour and 4 hours, between about 1 hour and 2 hours, between about 3 hours and 16 hours, between about 5 hours and 16 hours, between about 6 hours and 16 hours, between about 8 hours and 16 hours, between about 10 hours and 16 hours, between about 12 hours and 16 hours, between about 14 hours and 16 hours, or about 1 hour, or about 2 hours, or about 4 hours, or about 6 hours, or about 8 hours, or about 10 hours, or about 12 hours, or about 14 hours, or about 16 hours. The reaction product may be purified by aqueous work-up followed by chromatography.

Step (c) may be performed under cross coupling reaction conditions known to the person skilled in the art. In particular, it may involve a cross coupling catalyst. The catalyst may be selected from a palladium-containing catalyst. It may be selected from the group consisting of Pd(PPh₃)₄ or 1,1′-[bis(diphenylphosphino)ferrocene]dichloropalladium(II) complex with dichloromethane. It may involve a base selected from the group of nitrogen bases. It may involve bases selected from the group of inorganic bases. For example, triethylamine, pyridine, sodium hydroxide, potassium hydroxide or sodium bicarbonate may be used. A solvent mixture may be used. The solvent mixture may contain water. The solvent mixture may contain a polar solvent. The solvent mixture may be a mixture of water and 1,4-dioxane. The reaction temperature may be in the range of about 60 to 150° C., or to about 80 to 150° C., or to about 100 to 150° C., or to about 120 to 150° C., or to about 140 to 150° C., or to about 60 to 130° C., or to about 60 to 110° C., or to about 60 to 90° C., or to about 60 to 70° C., or to about 60° C., or to about 80° C., or to about 100° C., or to about 110° C., or to about 130° C., or to about 150° C. The reaction may be performed under conditions known to the person skilled in the art. The reaction product may be purified by filtration followed by chromatography.

A process for synthesizing the compound as disclosed above having the following formula (IV), comprises the steps of;

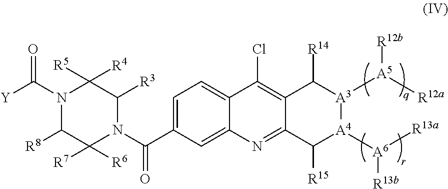

(a) contacting an amino substituted terephthalic acid or an ester thereof; with an optionally substituted cyclic ketone having the following Formula (VIII) to form a cyclized product;

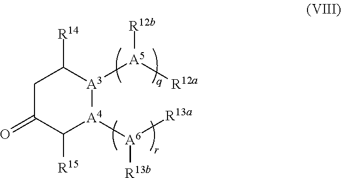

(b) selectively displacing at least one ketone of the cyclized product of step (a) with a halogen;

(c) optionally selectively hydrolyzing the ester of step (a) to a carboxylic acid; and

(d) selectively functionalizing the carboxylic acid of the cyclized product of step (a) or step (c) with a group having the following formula (VI) under reaction conditions to form the compound of formula (IV);

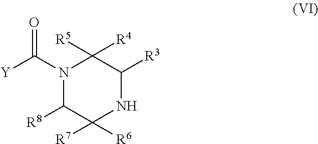

wherein step (b), (c) and (d) may be performed simultaneously, sequentially or in any order.

The reaction steps may be described as disclosed above.

The process comprises the step of optionally hydrolyzing the carboxylic acid ester after formation of the cyclized product in step (a). The hydrolyzing step may be performed under conditions known to the person skilled in the art.

By way of illustration, the compounds, esters, amides, salts and solvates of Formula (IV) may be prepared by a process which comprises an initial reaction step (a) between a terephthalic acid or an ester therof, and a carbonyl-containing moiety of Formula (VIII). This reaction may be carried out in a solvent. It may occur in a high-boiling solvent. The solvent may be selected from the group consisting of toluene, 1,4-dioxane, n-butanol, diphenyl ether, chlorobenzene, carbon tetrachloride, diethylene glycol, diglyme, hexamethylphosphoramide, o-xylene, m-xylene and p-xylene. The reaction temperature may be in a range of about 100 to about 400° C., or about 150 to about 400° C., or about 200 to about 400° C., or about 250 to about 400° C., or about 300 to about 400° C., or about 350 to about 400° C., or about 150 to about 350° C., or about 150 to about 300° C., or about 150 to about 250° C., or about 150 to about 200° C., or about 150 to about 350° C., or about 200 to about 300° C., or about 250 to about 300° C., e.g. at about 100° C., at about 150° C., at about 200° C., at about 250° C., at about 300° C., at about 350° C., or at about 400° C. It may be heated in a sealed tube. It may be in a reflux apparatus. It may be heated by an oil bath or a sand bath. It may be heated using microwave irradiation. The reaction time may vary between 30 min to 6 hours. It may vary in a range of about 30 min to 6 hours, or about 1 hour to 6 hours, or about 1.5 hours to 6 hours, or about 2 hours to 6 hours, or about 2.5 hours to 6 hours, or about 3 hours to 6 hours, or about 3.5 hours to 6 hours, or about 4 hours to 6 hours, or about 5 hours to 6 hours, or about 30 min to 5 hours, or about 30 min to 4 hours, or about 30 min to 3 hours, or about 30 min to 2 hours, or about 30 min to 1 hour, e.g. it may be about 30 min, or about 1 hour, or about 2 hours, or about 3 hours, or about 4 hours, or about 5 hours, or about 6 hours. After the reaction is complete, that reaction solution may be diluted with a non-polar solvent. The non-polar solvent may be selected from pentane, hexane, heptane, methyl t-butyl ether, petroleum ether and dichloromethane. The product may precipitate out of the solution.

In reaction step (b) the ensuing amino-enone may be treated with a halogenating reagent. The halogenating reagent may be phosphoryl-containing. It may be phosphorous oxychloride. Alternatively, it may be oxalyl chloride. The reaction may be in a solvent. It may be in a non-polar solvent. It may be in a solvent selected from the group consisting of hexane, cyclohexane, benzene, toluene, 1,4-dioxane, chloroform, diethyl ether and dichloromethane. It may be at an elevated temperature. The temperature may be in a range of about 30 to 120° C., or about 50 to 120° C., or about 70 to 120° C., or about 90 to 120° C., or about 110 to 120° C., or about 30 to 100° C. or about 30 to 80° C., or about 30 to 60° C., or about 30 to 40° C. or at about 30° C., or at about 50° C., or at about 70° C., or at about 90° C., or at about 110° C., or at about 130° C. The reaction time may be between about 30 min and 4 hours, or between about 1 hour and 4 hours, or between about 2 hours and 4 hours, or between about 3 hours and 4 hours, or between about 30 min and 3 hours, or between about 30 min and 2 hours, or between about 30 min and 1 hour, or about 30 min, or about 1 hour, or about 2 hours, or about 3 hours, or about 4 hours. The reaction product may be purified by aqueous work-up followed by chromatography.

For the hydrolysis of reaction step (c), the ester-containing starting material may be treated with a base in a solvent. The base may be selected from a variety of bases including inorganic bases or nitrogen bases. For example, triethylamine, pyridine, sodium hydroxide, potassium hydroxide, lithium hydroxide or sodium bicarbonate may be used. For example, the base may be lithium hydroxide. The solvent mixture may contain of two solvents. At least one of these solvents may be a polar solvent. The solvent mixture may contain methanol. The solvent mixture may contain 1,4-dioxane. The solvent mixture may be a mixture, for example, of methanol and 1,4-dioxane. The reaction may be followed by an aqueous work-up under acidic conditions. The pH of the aqueous work-up may be adjusted to about 2, about 3, about 4, or about 5, it may be, for example, 3.

The selective functionalizing of the carboxylic acid of step (d) may be performed under peptide coupling reaction conditions known to the person skilled in the art. In particular, it may involve a peptide coupling reagent selected from the group consisting of HATU, N,N′-dicyclohexylcarbodiimide, HBTU, hydroxybenzotriazole, propyl phosphonic anhydride and phosphorous oxychloride. It may involve a base selected from the group of nitrogen bases. It may involve bases selected from the group of inorganic bases. For example, triethylamine, pyridine, sodium hydroxide, potassium hydroxide or sodium bicarbonate may be used. A solvent may be used. The solvent may include polar aprotic solvents. The solvent may be selected from the group consisting of tetrahydrofuran, ethyl acetate, acetone, DMF, acetonitrile, dimethylsulfoxide or nitromethane. The reaction may be performed at room temperature. The reaction time may be between about 1 hour and 16 hours, or between about 1 hour and 14 hours, or between about 1 hour and 12 hours, or between about 1 hour and 10 hours, or between about 1 hour and 8 hours, or between about 1 hour and 6 hours, between about 1 hour and 4 hours, between about 1 hour and 2 hours, between about 3 hours and 16 hours, between about 5 hours and 16 hours, between about 6 hours and 16 hours, between about 8 hours and 16 hours, between about 10 hours and 16 hours, between about 12 hours and 16 hours, between about 14 hours and 16 hours, or about 1 hour, or about 2 hours, or about 4 hours, or about 6 hours, or about 8 hours, or about 10 hours, or about 12 hours, or about 14 hours, or about 16 hours. The reaction product may be purified by aqueous work-up followed by chromatography.

For the purposes of the processes described above, the term “at least one ketone” refers to 1, 2 or 3 ketone moieties and the term “at least one halogen” refers to 1, 2 or 3 halogen moieties.

The compounds as defined above may be made according to the general processes as disclosed above or according to the general principles of the working examples.

### EXAMPLES

Non-limiting examples of the disclosure will be further described in greater detail by reference to specific Examples, which should not be construed as in any way limiting the scope of the invention.

### Example 1

List of abbreviations used

| Names/terms | Abbreviations |
|---|---|
| Dichloromethane | DCM |
| Dimethylformamide (N,N-) | DMF |
| Dimethyl sulfoxide | DMSO |
| equivalent | equiv |
| High-performance liquid chromatography or | HPLC |
| high-pressure liquid chromatography | |
| Nuclear Magnetic Resonance | NMR |
| Tetrahydrofuran | THF |

Materials and Methods of the Biological Assays

SMYD3 Biochemical Assay

A SMYD3 enzymatic assay was developed using Promega's Methyltransferase-Glo™ reagents. In the assay, SMYD3 catalyzes the methylation of the MAP3K2 peptide substrate by transferring a methyl group from SAM to MAP3K2 peptide and further converts the SAM to SAH. The SMYD3 methyltransferase activity is measured based on the amount of SAH produced from the reaction through the use of coupling enzymes that convert the SAH to ATP. The MTase-Glo detection solution then catalyzes the formation of light from ATP.

For the IC₅₀ determination, the compounds were incubated with 0.4 μM of SMYD3 enzyme for 30 min in low volume 384 well plates. A final concentration of 1.0 μM and 10 μM SAM and MAP3K2 peptide were added and further incubated for 30 min at room temperature before adding the MTase Glo and detection reagent. Reaction signals were detected using microplate readers on luminescent mode (Safire Tecan). The IC₅₀ was determined by non-linear regression, using GraphPad Prism version, 5.03.

Cells and Reagents

The cell proliferation assays were tested in several cell lines, including HepG2,HCT116, A549, HPAF-II, CFPAC-1,HuH7, SNU398,Hep3B, and HEK293. All cell lines are from ATCC. HepG2, HPAF-II and HEK293 are cultured in Eagle's MEM media supplemented with fetal bovine serum. HCT116 is cultured in McCoy media supplemented with fetal bovine serum. Huh-7 is cultured in DMEM low glucose (1000 mg/L glucose) with 10% FBS, 1% L-Glutamate and 1% Penicillin/Streptomycin. SNU398 is cultured in RPMI with 10% FBS with 1% L-Glutamate and 1% Penicillin/Streptomycin. Hep3B is cultured in Eagle's MEM with 10% FBS, 1% L-Glutamate and 1% Penicillin/Streptomycin. A549 is cultured in RPMI media supplemented with fetal bovine serum while CFPAC-1 is cultured in IMDM media supplemented with fetal bovine serum. All media and serum are purchased from Gibco (Lifetech). All cells were grown in a temperature controlled incubator at 37° C. and 5% CO₂.

Cell Proliferation Assay

Cell proliferation assay was performed using CellTiter-Glo Luminescent Cell Viability Assay (Promega) following manufacturer's instructions. The cell-line of interest was treated with compounds that were serial diluted in its respective media. Plates were incubated for 3 days at 37° C. in 5% CO₂. After 3 days, an equal volume of Cell Titer Glo reagent was added. Plates were rocked on a rotator for 2 h. 100 μL of each well were transferred to a 96-well opaque plate, and luminescence emitted was measured with the Tecan Safire II.

Target Engagement Assay

The target engagement assay was performed with HEK293 that is engineered to overexpress SMYD3 (HEK293-SMYD3). The plasmid SMYD3 (Myc-DDK-tagged-Human SET and MYND domain containing 3) was purchased from Origene (RC230064) and transfected with lipofectamine 2000 (Invitrogen) into HEK293 (ATCC). The cell line is cultured in Eagle's MEM media supplemented with fetal bovine serum and geneticin (Invitrogen). Presence of over-expressed SMYD3 is confirmed with western blot with antibody against SMYD3 and the MYC tag as well as with RT-PCR with SMYD3 primers.

The cells were seeded in 6 well plates. After seeding for 24 h, the cells were treated with either DMSO or 25 μM compound and incubated for 24 hr. The cells were trypsinized and the lysate was extracted with RIPA buffer (Santa Cruz). The total protein concentration of lysate is quantified using the standard Bradford assay (Biorad protein assay, microplate standard assay).

Western Blot Analysis

Western blot analysis was performed using antibody against SMYD3 in HEK293 cells over-expressing SMYD3 (HEK293-SMYD3) treated with compound at different time points. 15.0 μg of cell lysate was diluted in 2× Laemmli sample buffer (Bio-Rad) and boiled at 100° C. on heat block for 5 min. Lysates were separated using NuPAGE® 4-12% Bis-Tris precast polyacrylamide gels (Lifetech) at 200 V, 400 A for 40 min. The electrophoresed protein was transferred onto the nitrocellulose membrane for 7 min using the iBlot 2 Dry Blotting System (Lifetech) . After 1 h incubation in blocking buffer [PBS (phosphate buffered saline) with 0.1% Tween 20 and 5% dry milk], the membrane was probed with anti-SMYD3 primary antibody mouse [GT1088] (Ab 177163, Abcam), 1:2500 dilution in PBS, 0.1% Tween 20 and 5% dry milk overnight at 4° C., followed by three washes (15 min each wash) in PBS, 0.1% Tween 20 on the next day. This was further continued with 1 h incubation with peroxidase-conjugated secondary antibody (anti-mouse-HRP, NA9310V (GE)), 1:5000 dilutions in PBS, 0.1% Tween 20 and 5% dry milk followed by three washes (15 min each wash) in PBS, 0.1% Tween 20. The nitrocellulose membrane was developed with enhanced Chemiluminescence (ECL) mixture (Amersham, Aylesbury, UK), incubated for 5 min and exposed using FluorChem E System instrument (Protein Simple).

Western blot analysis for detecting methylated MAP3K2 was carried out using a customized Anti-me2/me3-Lys 260 MAP3K2 at 1:500 and the total MAP3K2 was detected using Anti-MEKK2 (AB33918) using a 1:10,000 dilution.

Soft Agar Colony Formation Assay

Hep G2 cells were purchased from ATCC. Hep G2 cells were maintained in Eagle's Minimum Essential Medium (Sigma, Cat No: #M0643) supplemented with 10% Fetal Bovine Serum (Hyclone, Cat No: SV30087.03), 2 mM L-glutamine, 100 units/mL penicillin and 100 μg/mL streptomycin (Life Technologies, Cat No: 10378-016). The soft agar assays were performed in concordance to the ETC approved method report for soft agar assay (ETC document number: RD0019). Briefly, 600 μL of 0.6% agar was added to 24-well plate (Corning, Cat No: 3738) to form the base layer. This is followed by the addition of 500 μL of 0.36% agar middle layer (containing 10′000Hep G2 cells). Lastly, 500 μL of fresh growth medium (containing the corresponding serially diluted compound) was added above the middle layer. The plates were incubated at 37° C. with 5% carbon dioxide in a humidified incubator for 1 to 2 weeks. 70 μL of thiazolyl blue tetrazolium bromide (5 mg/mL, Sigma Cat No: M5655) was added to each well and the plates were incubated at 37° C. for 2 h. Colonies were counted with GelCount® instrument (Oxford Optronix). The colony counts were plotted against compound concentrations using the Graphpad Prism software. In addition, the software was used to perform non-linear curve fitting and the calculation of IC₅₀.

### Example 2

SMYD3 Biochemical Assay

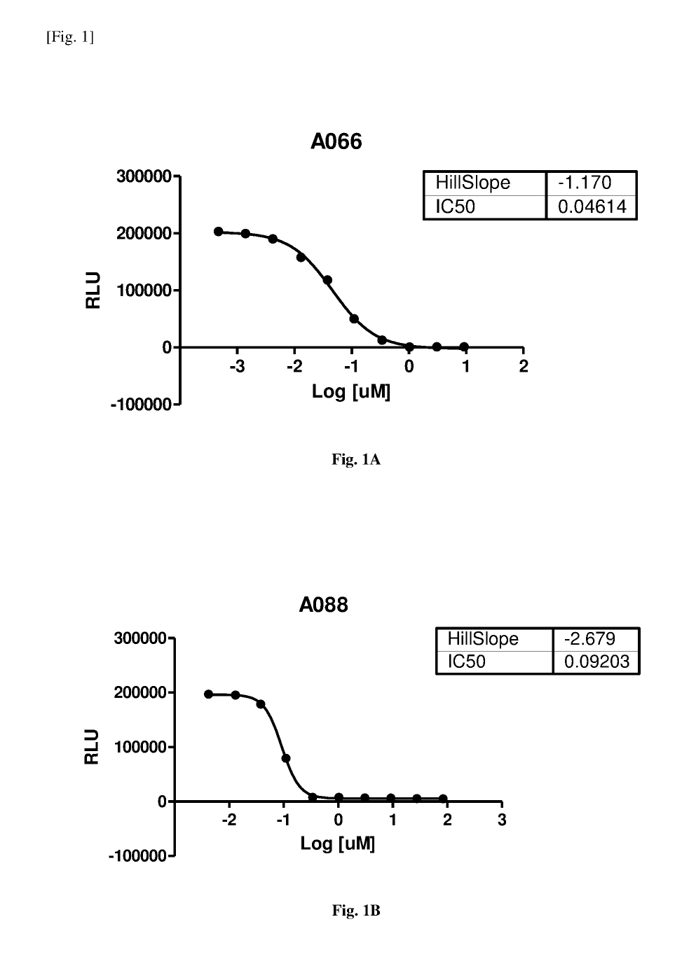

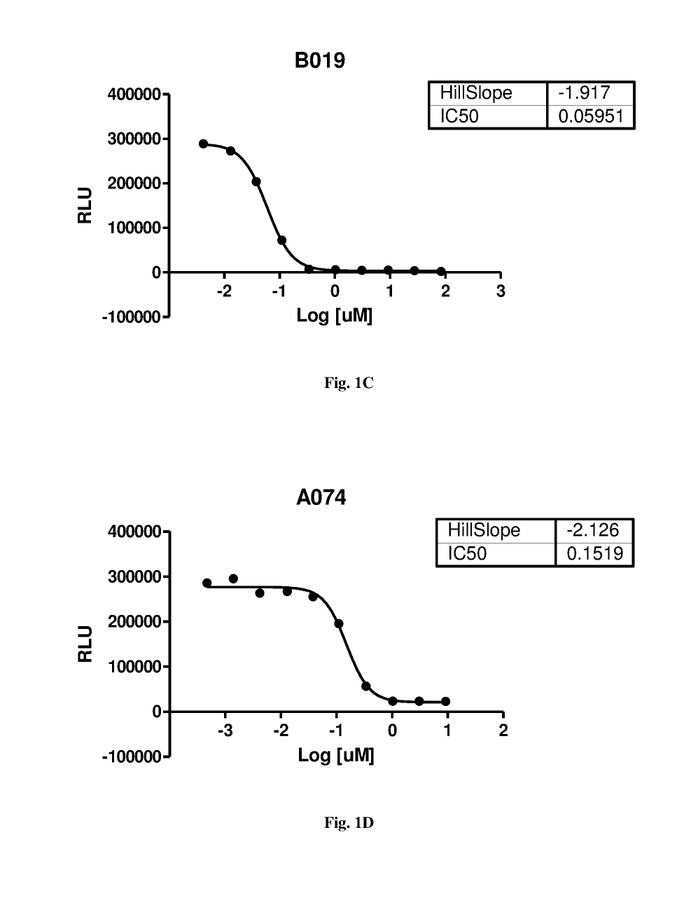

The ability of compounds to inhibit the catalytic function of SMYD3 was tested using the MTase assay by using the MAP3K2 as a peptide substrate. The compounds were found to inhibit the methyltransferase activity of SMYD3. The effect of compounds on the methyltransferase activity of SMYD3 using MAP3K2 peptide as a substrate can be found in FIG. 1 (Compound A066; A074; B019; A088). The data has been summarized in Table 1.

TABLE 1
Summary of biochemical IC₅₀ of SMYD3 inhibitors.

| Compound | A066 | A074 | B019 | A088 |
|---|---|---|---|---|
| IC₅₀ (μM) | 0.04614 | 0.1519 | 0.05951 | 0.09203 |

### Example 3

SMYD3 Compounds Inhibit the Proliferation of Different Cancer Cells

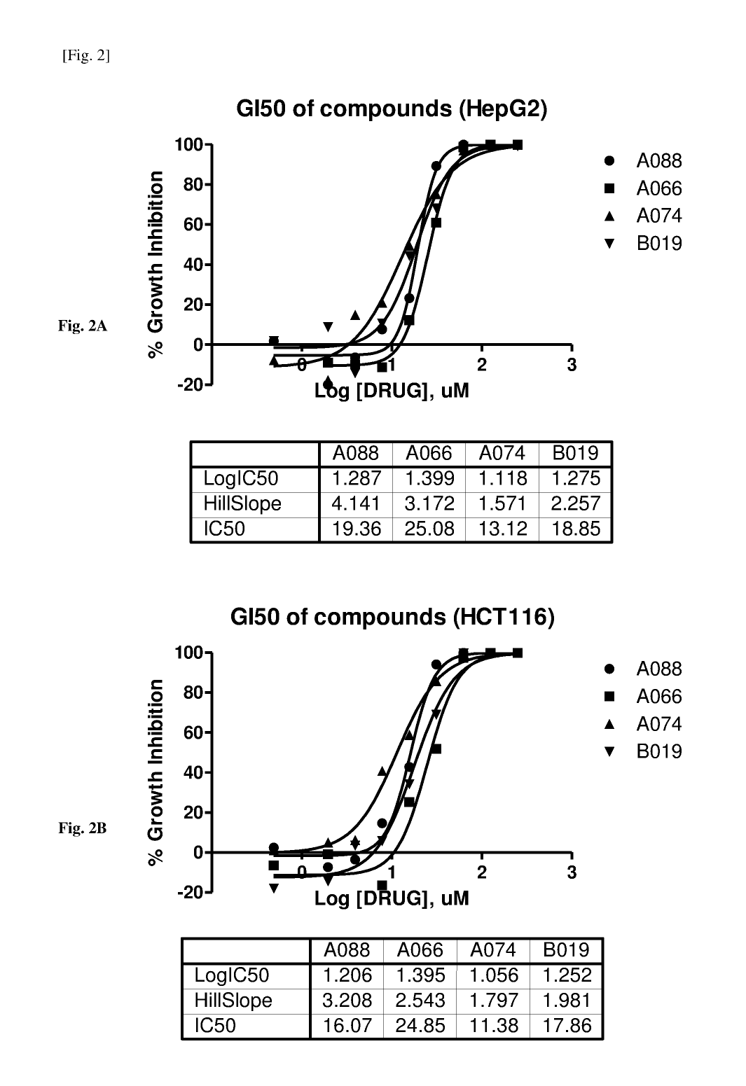

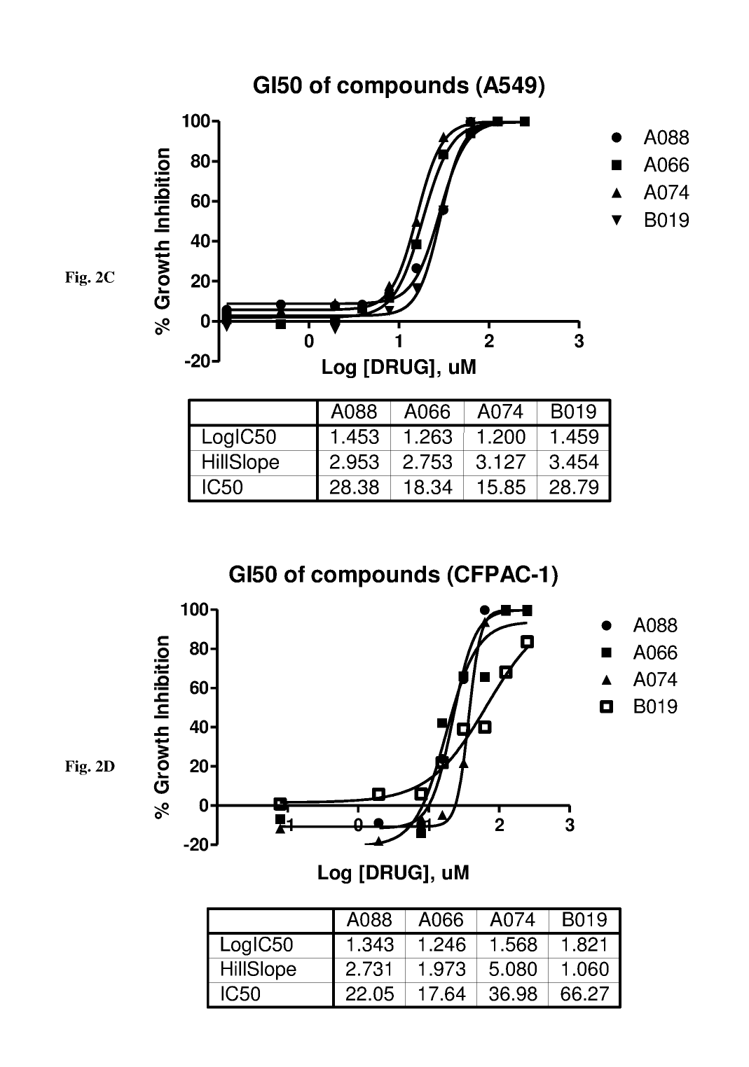

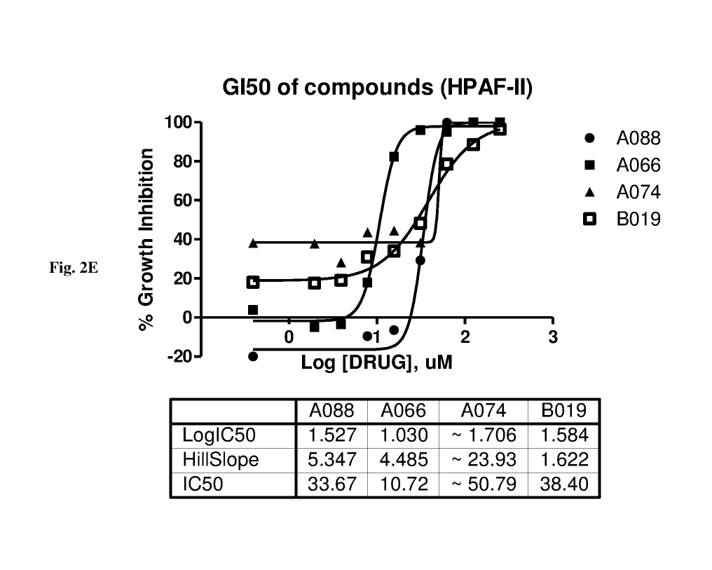

The anti-proliferative effects of SMYD3 inhibition were explored in different cancer cell lines. The dose-response curves are shown in FIG. 2. All cell lines responded to the SMYD3 inhibitors with GI₅₀ values ranging from 11 to 50 μM. A subset of the data is summarized in Table 2.

TABLE 2
Summary of anti-proliferative activity of SMYD3 inhibitors in different cancer cell
lines (Compounds A066; A074; B019; A088).

| | Biochemical | | | | | |
|---|---|---|---|---|---|---|
| | assay | HepG2 | HPAF-II | CFPAC-1 | HCT-116 | A549 |
| Compound | IC₅₀ (μM) | GI₅₀ (μM) | GI₅₀ (μM) | GI₅₀ (μM) | GI₅₀ (μM) | GI₅₀ (μM) |
| A088 | 0.09203 | 17.05 | 33.7 | 21.94 | 16.07 | 28.38 |
| A066 | 0.04614 | 33.08 | 11.13 | 15.35 | 24.85 | 18.4 |
| B019 | 0.05951 | 18.85 | *ND | *ND | 17.86 | 28.79 |
| A074 | 0.1519 | 10.65 | 50.4 | 36.9 | 11.38 | 15.85 |

*ND: not determined

### Example 4

SMYD3 Compounds Inhibit the SMYD3 Mediated Methylation of MAP3K2 in Cells

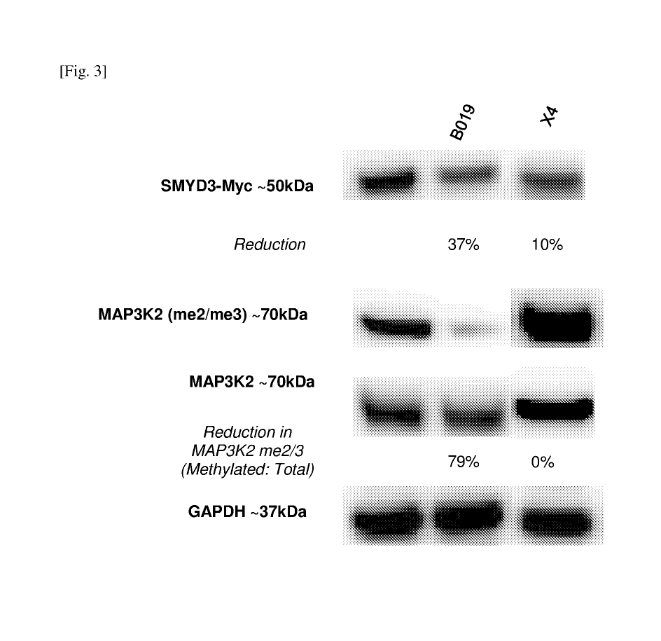

SMYD3 target engagement with B019 was demonstrated in HEK293 cells transiently transfected with Myc-tagged SMYD3. The cells were treated with 25 μM of the compound, overnight before analyzing the lysate on Western Blot. A 35-37% reduction in SMYD3 levels was observed using an antibody (Anti-SMYD3 #Ab177163) (1:5000) against SMYD3, as shown in FIG. 3.

B019 was further tested for its ability to inhibit SMYD3 in cells through its effects on cellular MAP3K2 methylation. This was performed using an antibody against the MAP3K2 (me2/me3), Anti-ME2/ME3-K260-MAP3K2 (1:500) and total MAP3K2, Anti-MEKK2 #ab33918 (1:10,000). B019 inhibited the methylation of MAP3K2 in the cells without changing the total MAP3K2 levels. Whereas, the less active compound X4 (please refer to Comparative Example 1) was unable to inhibit the methylation of MAP3K2 at 25 μM.

### xample 5

Inhibition of Anchorage Independent Growth of Cancer Cells with SMYD3 Inhibitors

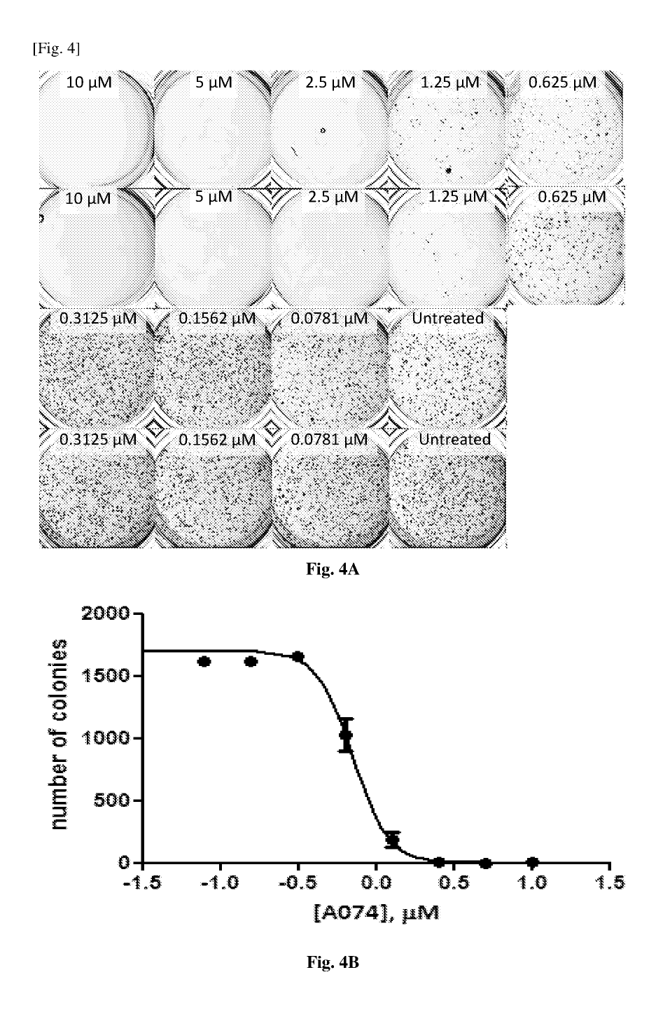

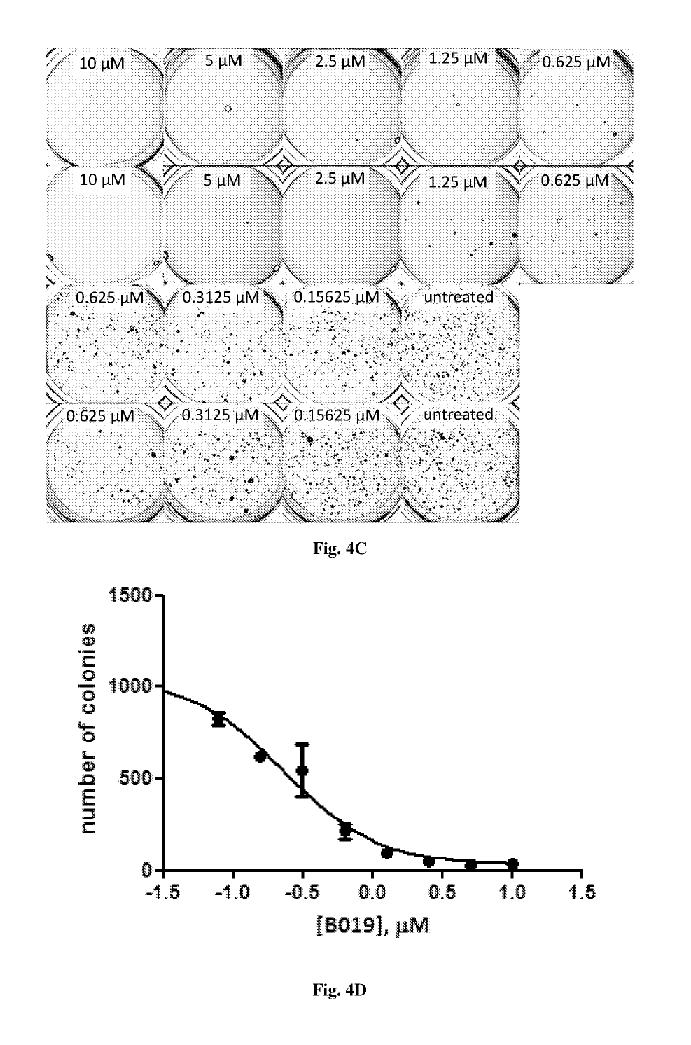

Compound A074 and B019 inhibited colony formation in Hep G2 cell line in the soft agar assay (FIG. 4). At the highest concentration tested (10 μM), both compounds inhibited about 100% of HepG2 colony formation. Each data point in the dose response curve consists of average colony count from two wells and the error bar is the standard deviation of the count. FIG. 4B shows an IC₅₀ of 0.71 μM and a maximum inhibition of 100%, FIG. 4D shows an IC₅₀ of 0.23 μM and maximum inhibition of 97.2%.

### Example 6

General Reaction Schemes

General Procedures. All reactions were performed using oven-dried round-bottomed flasks or reaction vessels. Where appropriate, reactions were carried out under an inert atmosphere of nitrogen with dry solvents, unless otherwise stated. Dry dichloromethane (DCM), tetrahydrofuran (THF), N,N-dimethylformamide (DMF), dimethylsulfoxide (DMSO), toluene (PhMe), acetonitrile (MeCN) and methanol (MeOH) were purchased at the highest commercial quality. Yields refer to chromatographically and spectroscopically (¹H NMR) homogeneous materials, unless otherwise stated. Reagents were purchased at the highest commercial quality and used without further purification, unless otherwise stated. Reactions were monitored by thin-layer chromatography carried out on 0.25 mm E. Merck silica gel plates (60F-254) using ultraviolet light as visualizing agent and potassium permanganate and heat as developing agents. NMR spectra were recorded on a Bruker/Agilent 400 spectrometer and were calibrated using residual undeuterated solvent as an internal reference (CDCl₃: NMR=7.26; DMSO-d_{6:} ¹H NMR=2.50; CD₃OD: ¹H NMR=3.31). The following abbreviations or combinations thereof were used to explain the multiplicities: s=singlet, d=doublet, t=triplet, q=quartet, sex=sextet, m=multiplet, br=broad. Liquid chromatography mass spectra (LCMS) were recorded on an Agilent or Shimadzu mass spectrometer using ESI-TOF (electrospray ionization-time of flight).

1. General Procedure for the Synthesis of Compounds with General Formula (IV).

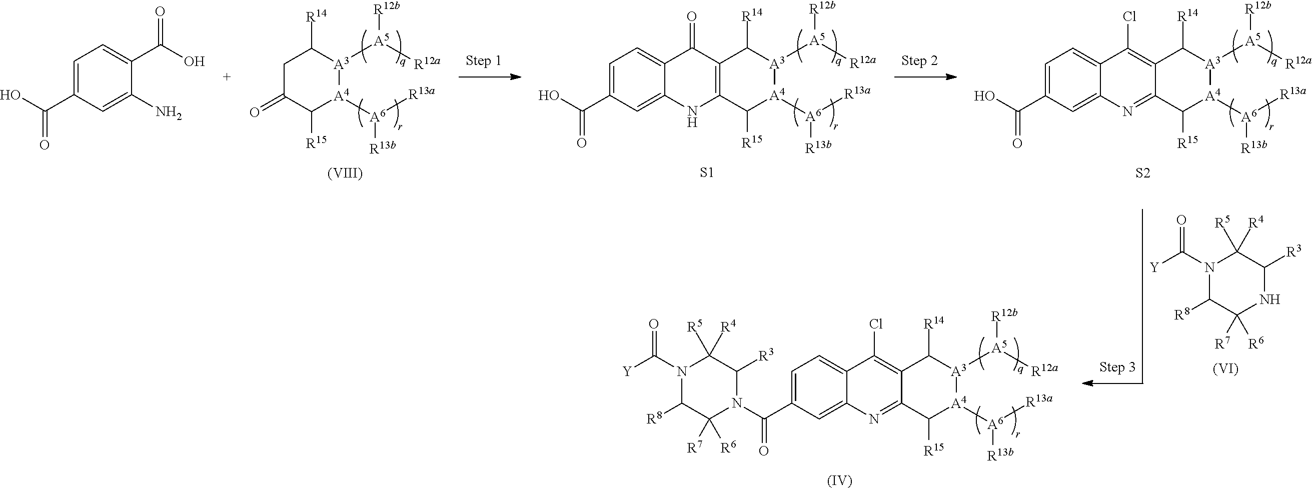

Step 1: General Procedure A

A solution of 2-amino-terephthalic acid (1 equiv) and cyclic ketone (VIII) (1.2 equiv) in diphenyl ether (10 mL/g) was heated to 300° C. for 1-3 h in a sealed tube. Upon cooling to room temperature, the reaction was diluted with hexanes and the resulting solid was collected by filtration to afford cyclized product S1. Cyclized product S1 was used without subsequent purification.

Step 2: General Procedure B

Cyclized product S1 (1 equiv) was treated with phosphorus oxychloride (10 mL/g) and the mixture was heated at 100° C. for 4 h. Upon cooling, the crude mixture was either: (a) concentrated under reduced pressure, diluted with cold water and stirred until a free solid formed. The solid was collected by filtration and washed with hexanes to afford compound S2, or: (b) diluted with dichloromethane and poured into a separating funnel containing cold 2 M aqueous sodium hydroxide. The organic layer was separated and the aqueous layer was extracted thrice with dichloromethane. The combined organic layers were washed with brine, dried over anhydrous sodium sulfate, filtered and concentrated under reduced pressure. The crude material was purified by column chromatography (ethyl acetate/hexanes) to afford chlorinated compound S2.

Step 3: General Procedure C

Condition 1: Amide Coupling using 1-[bis(dimethylamino)methylene]-1H-1,2,3-triazolo[4,5-b]pyridinium 3-oxid hexafluorophosphate (HATU)

Chlorinated compound S2 (1 equiv), amine (VI) (1-2 equiv) and triethylamine (2-4 equiv) were dissolved in N,N-dimethylformamide (0.1 M) and the solution was cooled to 0° C. HATU (1.5 equiv) was added and the reaction was quenched upon completion based on LCMS analysis (<1 h) by the addition of water. The aqueous layer was extracted 3-5 times with ethyl acetate, and the combined organic layers were washed with saturated sodium bicarbonate, brine, dried over anhydrous sodium sulfate, filtered and concentrated under reduced pressure. The crude material was purified by column chromatography to afford compound with general formula (IV).

Condition 2: Amide Coupling using Propylphosphonic Anhydride (T₃P)

To a suspension of compound S2 (1 equiv) and amine (VI) (0.5-1.2 equiv) in tetrahydrofuran (5-10 mL/g) at 0° C. was added T₃P (50% solution in ethyl acetate) (2-3 equiv) and triethylamine (10 equiv). The resulting mixture was stirred at room temperature for 2 h. The reaction mixture was diluted with ethyl acetate, and the organic layer was washed with water, brine, dried over anhydrous sodium sulfate, filtered and concentrated under reduced pressure. The crude product was purified by column chromatography (ethyl acetate/hexanes) or preparative HPLC to afford compound with general formula (IV).

2. General Procedure for Reduction of Compounds with Nitrile Group: General Procedure D

Potassium borohydride (4 equiv), Raney nickel (moist weight, approximately 1 equiv) were dissolved in dry ethanol (20 mL). To the resulting slurry was added compound to be reduced (1 equiv) while stirring. After vigorously stirring at room temperature for 45 min, the reaction mixture was filtered. The organic layer was evaporated and residue was dissolved in ethyl acetate. The resulting solution was washed with water and the organic layer was dried over anhydrous sodium sulfate and concentrated. The crude product was purified by flash column chromatography on silica gel (methanol/dichloromethane) followed by preparative-HPLC to get the desired product.

3. Alternative General Procedure for Compounds with General Formula (IV).

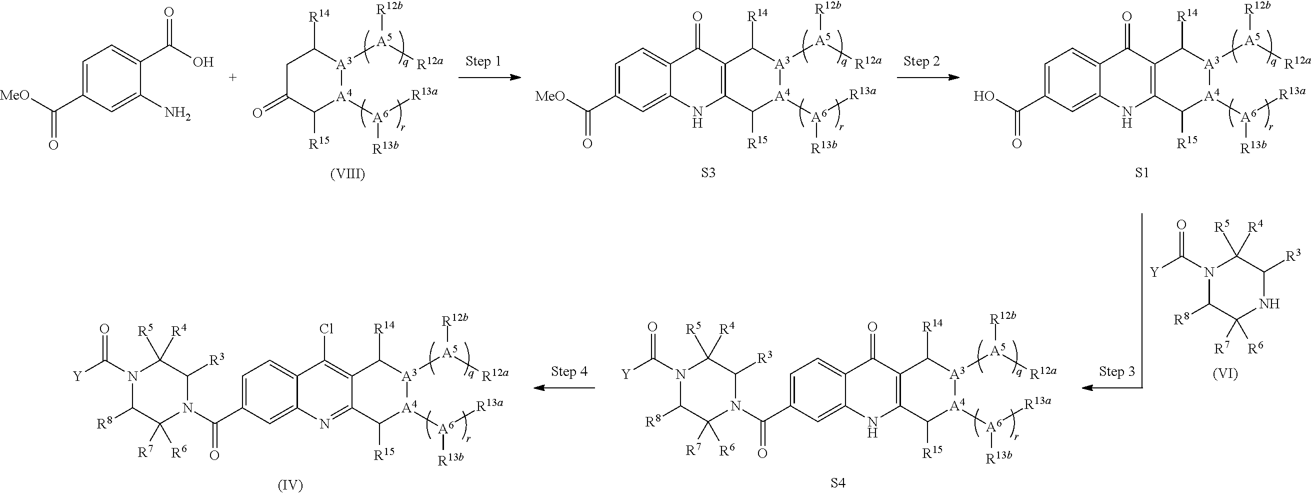

Step 1: General Procedure E

A solution of 2-amino-4-(methoxycarbonyl)benzoic acid (1 equiv) and cyclic ketone (VIII) (1.2 equiv) in diphenyl ether (10 mL/g) was maintained at 300° C. for 1-3 h in a sealed tube. The reaction mass was cooled to room temperature and diluted with hexanes. The resultant solid was collected by filtration, washed with hexanes and dried to afford cyclized product S3.

Step 2: General Procedure F

To a solution of Compound S3 (1 equiv) in a mixture of tetrahydrofuran, methanol and water (1:1:0.5 mL/g) was added lithium hydroxide monohydrate (3 equiv). The reaction mixture was stirred at room temperature for 2-5 h. The reaction mixture was concentrated under reduced pressure, the residue was diluted with water (20 mL), washed with EtOAc (3×50 mL). The aqueous layer was separated and acidified to a pH 2 using aqueous 2 M hydrochloric acid and the resulting solid was collected by filtration to afford compound S1.

Step 3: see General Procedure C for synthesis of S4.

Step 4: General Procedure G

A mixture of cyclized product S4, phosphorus oxychloride (5 mL/g) in dichloromethane (10 mL/g) was heated at 70° C. for 2 h. Upon cooling, the reaction mass was concentrated under reduced pressure and the reaction mass was diluted with ethyl acetate and washed with saturated sodium bicarbonate solution, brine and dried over anhydrous sodium sulfate, filtered, and concentrated. The crude product was purified by preparative-HPLC to afford compound with general formula (IV).

4. General Procedure for the Synthesis of Compounds with General Formula (III).

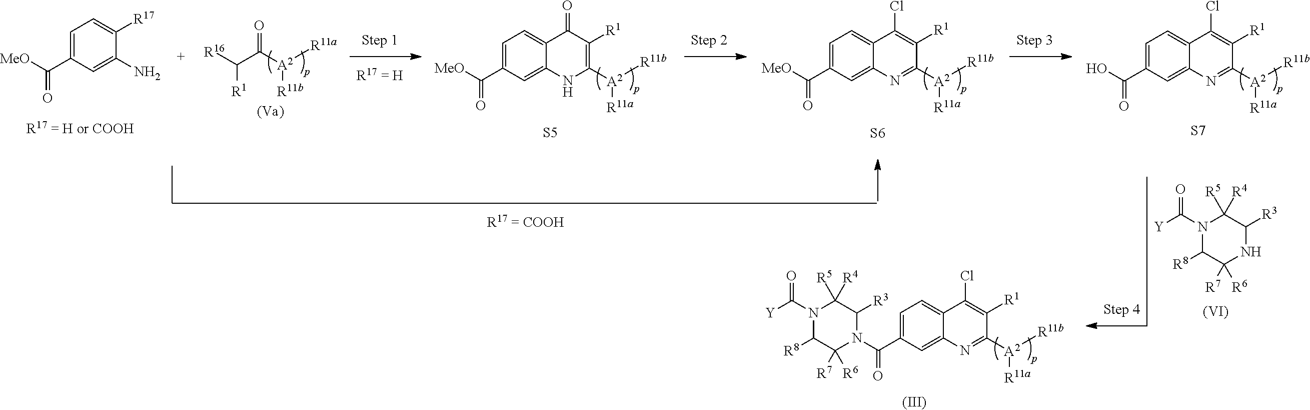

Step 1: General Procedure H

A solution containing compound (Va) (1 equiv), an optionally substituted aminobenzoate ester (R¹⁷=H, 1.2 equiv) and the hydrochloride salt of the aniline derivative (0.01 equiv) in toluene was heated to reflux in a Dean-Stark apparatus overnight. The solution was cooled to room temperature and concentrated under reduced pressure. The crude material was purified by column chromatography (ethyl acetate/petroleum ether) to afford the intermediate enamine This intermediate was dissolved in diphenyl ether (10 mL/g) and was heated to 330° C. for 2-4 h in a sealed tube. The reaction mass was cooled to room temperature and diluted with hexanes. The resulting solid was collected by filtration, washed with hexanes and dried to afford cyclized product S5.

Step 1A: General Procedure I

To a reaction vessel containing compound an optionally substituted aminobenzoate ester (R¹⁷=COOH, 1 equiv) and ketone (Va) (1-2 equiv) was added phosphorus oxychloride (1.6 mL/mmol). The resulting mixture was heated to 100° C. for 3 h before cooling it to 0° C. The cooled mixture was dissolved in dichloromethane (40 mL/mmol) and then basified by adding 2 M aqueous sodium hydroxide (40 mL/mmol). The organic layer was separated and the aqueous phase was extracted twice more with dichloromethane. The combined organic layers were washed with brine, dried over anhydrous sodium sulfate, filtered and concentrated under reduced pressure. The crude material was purified by column chromatography (ethyl acetate/hexanes) to afford compound S6 directly.

Step 2: General Procedure J

The solution of compound S5 (1 equiv) in phosphorus oxychloride (30 mL/mmol) was heated at 110° C. for 1 h. The reaction mixture was concentrated under reduced pressure and poured into ice water and the compound was extracted into 10% methanol in dichloromethane (2×100 mL). The combined organic layer was washed with water and brine, dried over anhydrous sodium sulfate, filtered and concentrated under reduced pressure to afford compound S6.

Step 3: General Procedure K

To a solution of S6 (1 equiv) in methanol (2 mL/mmol) and 1,4-dioxane (1 mL/mmol) was added an aqueous solution of lithium hydroxide (10 equiv) in water (2 mL/mmol). The mixture was heated to 50° C. for 1 h until all contents were soluble. The solution was cooled to room temperature before ethyl acetate (25 mL) was added. The mixture was acidified to pH 4 with 1 M aqueous hydrochloric acid and then the organic layer was separated. The aqueous layer was extracted thrice with ethyl acetate and the combined organic layer was dried over anhydrous sodium sulfate, filtered and concentrated under reduced pressure to afford compound S7.

Step 4: see General Procedure C to Synthesize Compounds with General Formula (III).

5. General Procedure 2 for Synthesis of Compounds with General Formula (III).

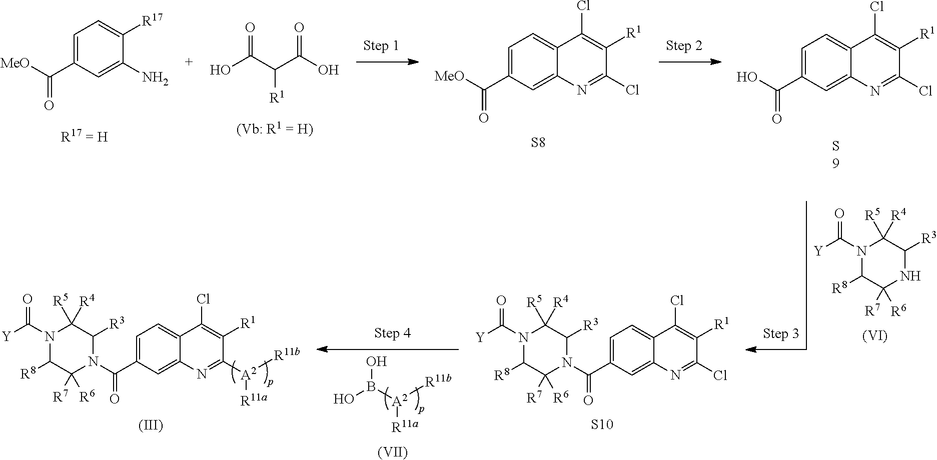

Step 1: Synthesis of Intermediate S8.

To a round-bottomed flask containing malonic acid (Vb) (11.36 g, 0.1092 mol, 1.1 equiv) was added phosphorus oxychloride (56 mL, 0.599 mmol, 6 equiv) dropwise at 0° C. After 30 min, methyl 3-aminobenzoate (15 g, 0.0992 mol) was added portion wise (3 additions) to the cold mixture. After warming to room temperature, the mixture was heated to reflux for 4 h. Upon cooling, the mixture was diluted with dichloromethane and neutralized to pH 14 using 6M aqueous sodium hydroxide. The organic layer was separated and the aqueous layer was extracted 4 more times with dichloromethane. The combined extracts were washed with saturated sodium bicarbonate, dried over anhydrous sodium sulfate, filtered and concentrated. The crude material was purified by column chromatography to afford S8 as a white solid (1.738 g, 7%).

Step 2: Synthesis of Intermediate S9.

Intermediate S8 (1.738 g, 6.96 mmol) was dissolved in methanol (21 mL) and 1,4-dioxane (21 mL). A solution of lithium hydroxide (1.7 g, 0.07095 mol, 10.2 equiv) in water (21 mL) was added. After 30 min, the mixture was acidified to pH 3 with concentrated hydrochloric acid. The organic solvents were removed under vacuum and the white residue was collected via filtration and washed with copious amount of water. The white solid was dried in a vacuum oven to afford intermediate S9 (1.549 g, 92%).

Step 3: Synthesis of Intermediate S10.

See General Procedure C.

Step 4: Synthesis of Compounds with General Formula (III). General Procedure L.

Intermediate S10 (1 equiv), boronic acid (VII) (1.3 equiv) and potassium phosphate tribasic (3 equiv) were added to a reaction vessel. A mixture of 1,4-dioxane/water (4:1) was added (0.07 M) and the resulting mixture was degassed with nitrogen. [1,1′-Bis(diphenylphosphino)ferrocene]dichloropalladium(II), complex with dichloromethane (0.1 equiv) was added and the resulting mixture was heated to 100° C. (30 min to 12 h). Upon cooling, the mixture was filtered through a pad of Celite® with ethyl acetate, and the mixture was concentrated. The product was purified either with column chromatography or preparative HPLC to afford compound with general formula (III).

### Example 7

The following Table 3 shows a list representing the exemplified compounds of this disclosure, together with the biological activity data. The ability of the exemplified compounds to inhibit the catalytic function of SMYD3 was tested using the MTase assay by using the MAP3K2 as a peptide substrate. The compounds were found to inhibit the methyltransferase activity of SMYD3.

Compound List

TABLE 3
Table listing the structure and IC₅₀ of the compounds disclosed

| Compound | Structure | IC₅₀ (μM) |
|---|---|---|
| A002 | 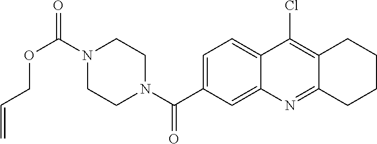 [SMILES: C=CCOC(=O)N1CCN(C(=O)c2ccc3c(Cl)c4c(nc3c2)CCCC4)CC1] | 1.0 |
| A001 | 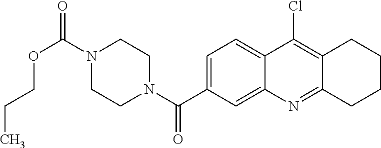 [SMILES: CCCOC(=O)N1CCN(C(=O)c2ccc3c(Cl)c4c(nc3c2)CCCC4)CC1] | 0.4 |
| A003 | 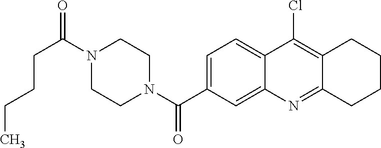 [SMILES: CCCCC(=O)N1CCN(C(=O)c2ccc3c(Cl)c4c(nc3c2)CCCC4)CC1] | 1.52 |
| C001 | 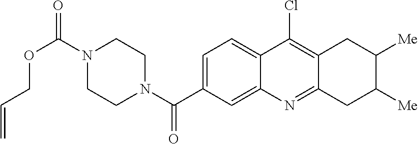 [SMILES: C=CCOC(=O)N1CCN(C(=O)c2ccc3c(Cl)c4c(nc3c2)CC(C)C(C)C4)CC1] | 7.7 |
| C002 | 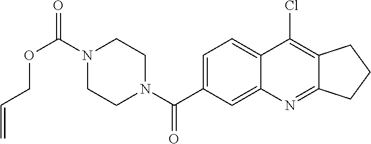 [SMILES: C=CCOC(=O)N1CCN(C(=O)c2ccc3c(Cl)c4c(nc3c2)CCC4)CC1] | 10.0 |
| A004 | 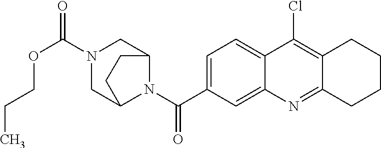 [SMILES: CCCOC(=O)N1CC2CCC(C1)N2C(=O)c1ccc2c(Cl)c3c(nc2c1)CCCC3] | 0.35 |
| A005 | 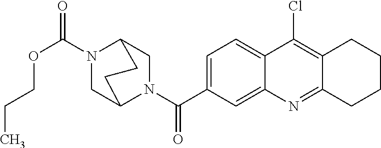 [SMILES: CCCOC(=O)N1CC2CCC1CN2C(=O)c1ccc2c(Cl)c3c(nc2c1)CCCC3] | 4.5 |
| A006 | 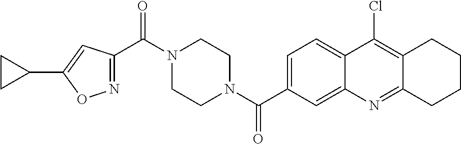 [SMILES: O=C(c1ccc2c(Cl)c3c(nc2c1)CCCC3)N1CCN(C(=O)c2cc(C3CC3)on2)CC1] | 6.7 |
| A007 | 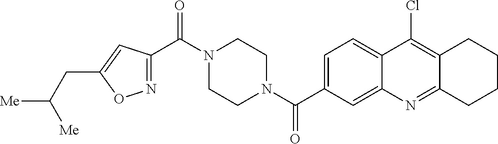 [SMILES: CC(C)Cc1cc(C(=O)N2CCN(C(=O)c3ccc4c(Cl)c5c(nc4c3)CCCC5)CC2)no1] | 6.9 |
| C003 | 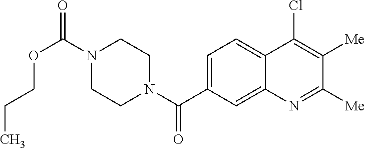 [SMILES: CCCOC(=O)N1CCN(C(=O)c2ccc3c(Cl)c(C)c(C)nc3c2)CC1] | 3.0 |
| A008 | 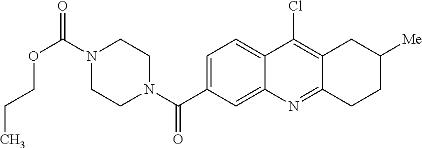 [SMILES: CCCOC(=O)N1CCN(C(=O)c2ccc3c(Cl)c4c(nc3c2)CCC(C)C4)CC1] | 1.3 |
| A009 | 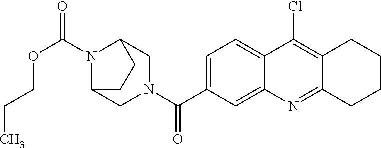 [SMILES: CCCOC(=O)N1C2CCC1CN(C(=O)c1ccc3c(Cl)c4c(nc3c1)CCCC4)C2] | 0.36 |
| A010 | 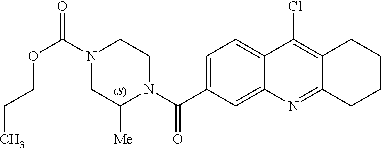 | 0.19 |
| A011 | 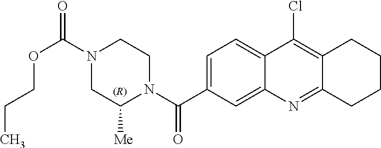 | 0.30 |
| A012 | 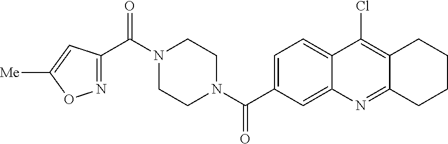 [SMILES: Cc1cc(C(=O)N2CCN(C(=O)c3ccc4c(Cl)c5c(nc4c3)CCCC5)CC2)no1] | 1.67 |
| A013 | 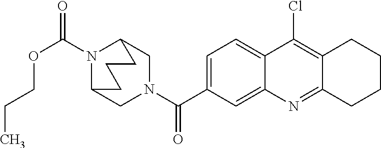 [SMILES: CCCOC(=O)N1C2CCCC1CN(C(=O)c1ccc3c(Cl)c4c(nc3c1)CCCC4)C2] | 0.19 |
| C004 | 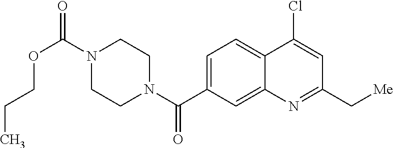 [SMILES: CCCOC(=O)N1CCN(C(=O)c2ccc3c(Cl)cc(CC)nc3c2)CC1] | 1.4 |
| A014 | 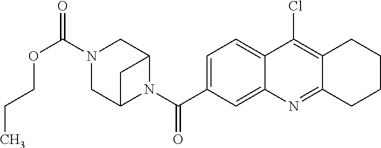 [SMILES: CCCOC(=O)N1CC2CC(C1)N2C(=O)c1ccc2c(Cl)c3c(nc2c1)CCCC3] | 1.0 |
| A015 | 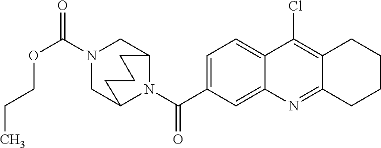 [SMILES: CCCOC(=O)N1CC2CCCC(C1)N2C(=O)c1ccc2c(Cl)c3c(nc2c1)CCCC3] | 0.32 |
| A016 | 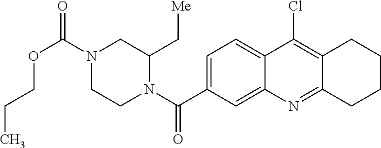 [SMILES: CCCOC(=O)N1CCN(C(=O)c2ccc3c(Cl)c4c(nc3c2)CCCC4)C(CC)C1] | 0.64 |
| C005 | 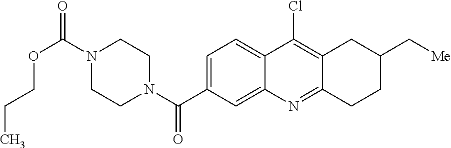 [SMILES: CCCOC(=O)N1CCN(C(=O)c2ccc3c(Cl)c4c(nc3c2)CCC(CC)C4)CC1] | 3.2 |
| A017 | 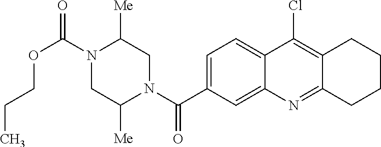 [SMILES: CCCOC(=O)N1CC(C)N(C(=O)c2ccc3c(Cl)c4c(nc3c2)CCCC4)CC1C] | 0.29 |
| A018 | 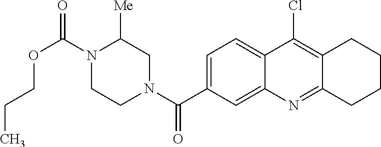 [SMILES: CCCOC(=O)N1CCN(C(=O)c2ccc3c(Cl)c4c(nc3c2)CCCC4)CC1C] | 0.39 |
| A019 | 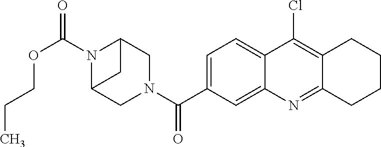 [SMILES: CCCOC(=O)N1C2CC1CN(C(=O)c1ccc3c(Cl)c4c(nc3c1)CCCC4)C2] | 13.1 |
| A020 | 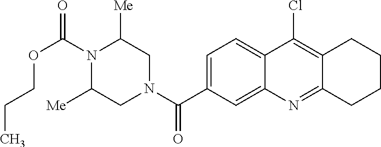 [SMILES: CCCOC(=O)N1C(C)CN(C(=O)c2ccc3c(Cl)c4c(nc3c2)CCCC4)CC1C] | 0.35 |
| A021 | 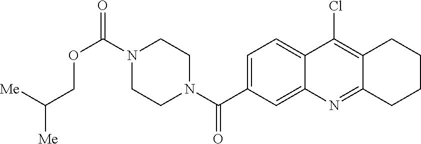 [SMILES: CC(C)COC(=O)N1CCN(C(=O)c2ccc3c(Cl)c4c(nc3c2)CCCC4)CC1] | 0.84 |
| A022 | 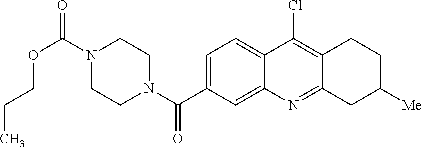 [SMILES: CCCOC(=O)N1CCN(C(=O)c2ccc3c(Cl)c4c(nc3c2)CC(C)CC4)CC1] | 1.0 |
| A023 | 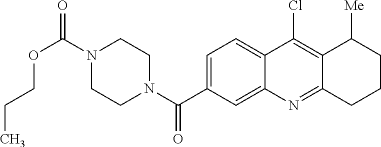 [SMILES: CCCOC(=O)N1CCN(C(=O)c2ccc3c(Cl)c4c(nc3c2)CCCC4C)CC1] | 5.2 |
| A024 | 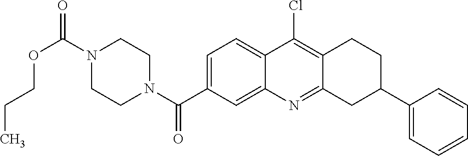 [SMILES: CCCOC(=O)N1CCN(C(=O)c2ccc3c(Cl)c4c(nc3c2)CC(c2ccccc2)CC4)CC1] | 1.2 |
| A025 | 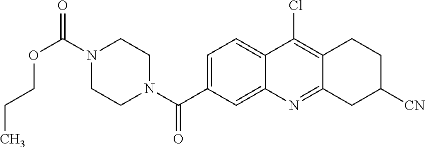 [SMILES: CCCOC(=O)N1CCN(C(=O)c2ccc3c(Cl)c4c(nc3c2)CC(C#N)CC4)CC1] | 1.9 |
| A026 | 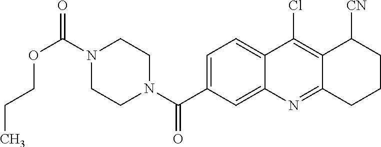 [SMILES: CCCOC(=O)N1CCN(C(=O)c2ccc3c(Cl)c4c(nc3c2)CCCC4C#N)CC1] | 3.2 |
| A027 | 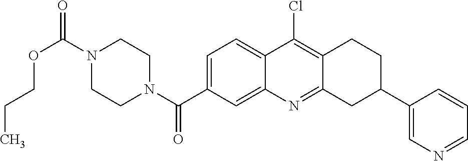 [SMILES: CCCOC(=O)N1CCN(C(=O)c2ccc3c(Cl)c4c(nc3c2)CC(c2cccnc2)CC4)CC1] | 0.40 |
| A028 | 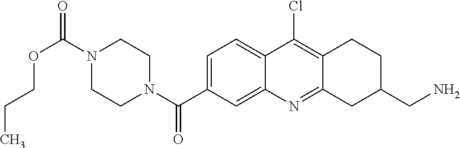 [SMILES: CCCOC(=O)N1CCN(C(=O)c2ccc3c(Cl)c4c(nc3c2)CC(CN)CC4)CC1] | 0.38 |
| A029 | 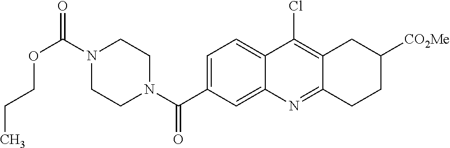 [SMILES: CCCOC(=O)N1CCN(C(=O)c2ccc3c(Cl)c4c(nc3c2)CCC(C(=O)OC)C4)CC1] | 0.86 |
| C006 | 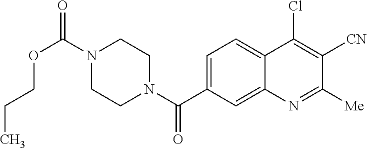 [SMILES: CCCOC(=O)N1CCN(C(=O)c2ccc3c(Cl)c(C#N)c(C)nc3c2)CC1] | 2.8 |
| C007 | 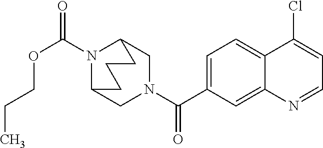 [SMILES: CCCOC(=O)N1C2CCCC1CN(C(=O)c1ccc3c(Cl)ccnc3c1)C2] | 0.55 |
| A030 | 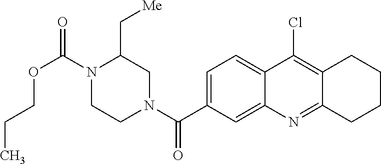 [SMILES: CCCOC(=O)N1CCN(C(=O)c2ccc3c(Cl)c4c(nc3c2)CCCC4)CC1CC] | 0.63 |
| A031 | 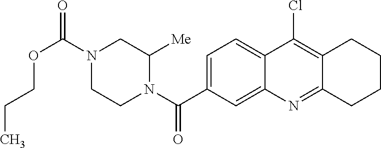 [SMILES: CCCOC(=O)N1CCN(C(=O)c2ccc3c(Cl)c4c(nc3c2)CCCC4)C(C)C1] | 0.15 |
| A032 | 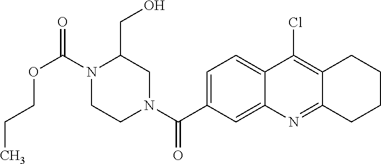 [SMILES: CCCOC(=O)N1CCN(C(=O)c2ccc3c(Cl)c4c(nc3c2)CCCC4)CC1CO] | 1.2 |
| A033 | 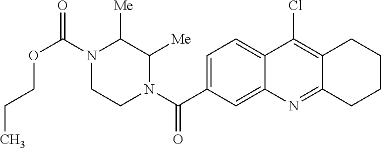 [SMILES: CCCOC(=O)N1CCN(C(=O)c2ccc3c(Cl)c4c(nc3c2)CCCC4)C(C)C1C] | 1.8 |
| A034 | 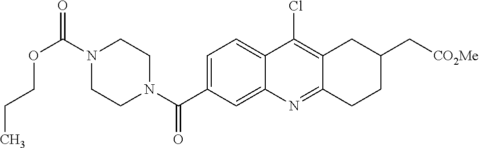 [SMILES: CCCOC(=O)N1CCN(C(=O)c2ccc3c(Cl)c4c(nc3c2)CCC(CC(=O)OC)C4)CC1] | 0.38 |
| A035 | 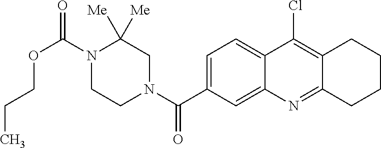 [SMILES: CCCOC(=O)N1CCN(C(=O)c2ccc3c(Cl)c4c(nc3c2)CCCC4)CC1(C)C] | 8.2 |
| A036 | 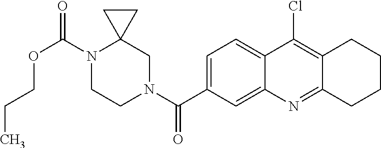 [SMILES: CCCOC(=O)N1CCN(C(=O)c2ccc3c(Cl)c4c(nc3c2)CCCC4)CC12CC2] | 6.7 |
| A037 | 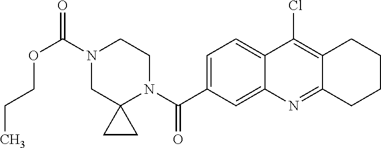 [SMILES: CCCOC(=O)N1CCN(C(=O)c2ccc3c(Cl)c4c(nc3c2)CCCC4)C2(CC2)C1] | 8.4 |
| A038 | 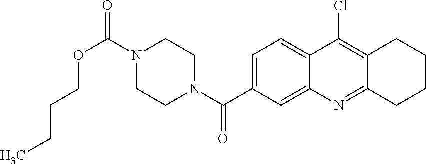 [SMILES: CCCCOC(=O)N1CCN(C(=O)c2ccc3c(Cl)c4c(nc3c2)CCCC4)CC1] | 0.42 |
| A039 | 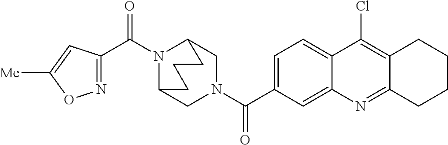 [SMILES: Cc1cc(C(=O)N2C3CCCC2CN(C(=O)c2ccc4c(Cl)c5c(nc4c2)CCCC5)C3)no1] | 1.3 |
| A040 | 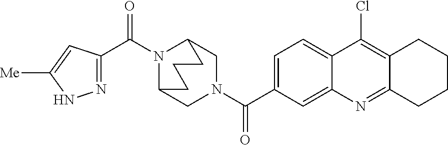 [SMILES: Cc1cc(C(=O)N2C3CCCC2CN(C(=O)c2ccc4c(Cl)c5c(nc4c2)CCCC5)C3)n[nH]1] | 10.9 |
| A041 | 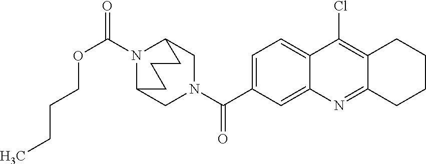 [SMILES: CCCCOC(=O)N1C2CCCC1CN(C(=O)c1ccc3c(Cl)c4c(nc3c1)CCCC4)C2] | 0.26 |
| A042 | 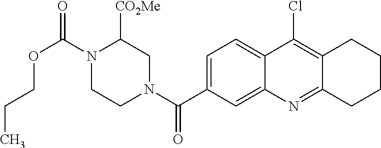 [SMILES: CCCOC(=O)N1CCN(C(=O)c2ccc3c(Cl)c4c(nc3c2)CCCC4)CC1C(=O)OC] | 5.2 |
| A043 | 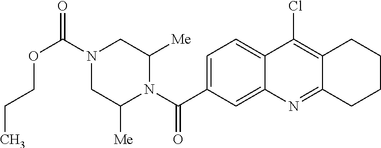 [SMILES: CCCOC(=O)N1CC(C)N(C(=O)c2ccc3c(Cl)c4c(nc3c2)CCCC4)C(C)C1] | 0.55 |
| A044 | 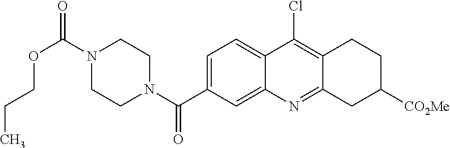 [SMILES: CCCOC(=O)N1CCN(C(=O)c2ccc3c(Cl)c4c(nc3c2)CC(C(=O)OC)CC4)CC1] | 0.62 |
| A045 | 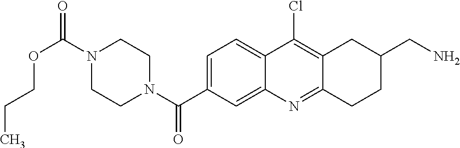 [SMILES: CCCOC(=O)N1CCN(C(=O)c2ccc3c(Cl)c4c(nc3c2)CCC(CN)C4)CC1] | 1.2 |
| A046 | 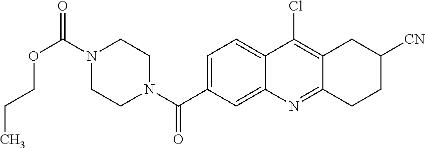 [SMILES: CCCOC(=O)N1CCN(C(=O)c2ccc3c(Cl)c4c(nc3c2)CCC(C#N)C4)CC1] | 0.80 |
| A047 | 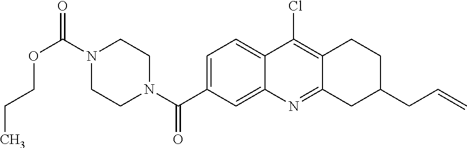 [SMILES: C=CCC1CCc2c(nc3cc(C(=O)N4CCN(C(=O)OCCC)CC4)ccc3c2Cl)C1] | 0.60 |
| B001 | 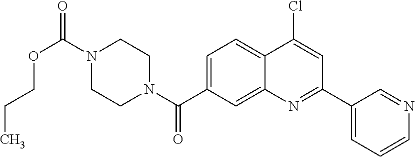 [SMILES: CCCOC(=O)N1CCN(C(=O)c2ccc3c(Cl)cc(-c4cccnc4)nc3c2)CC1] | 2.4 |
| B002 | 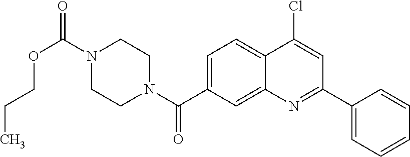 [SMILES: CCCOC(=O)N1CCN(C(=O)c2ccc3c(Cl)cc(-c4ccccc4)nc3c2)CC1] | 0.41 |
| A048 | 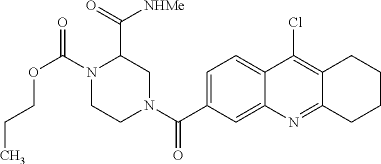 [SMILES: CCCOC(=O)N1CCN(C(=O)c2ccc3c(Cl)c4c(nc3c2)CCCC4)CC1C(=O)NC] | 6.1 |
| A049 | 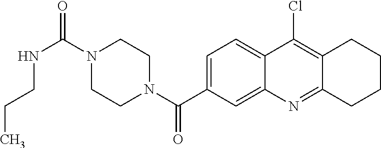 [SMILES: CCCNC(=O)N1CCN(C(=O)c2ccc3c(Cl)c4c(nc3c2)CCCC4)CC1] | 6.4 |
| A050 | 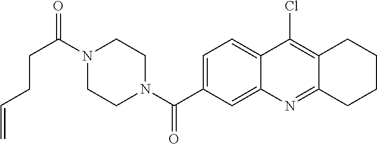 [SMILES: C=CCCC(=O)N1CCN(C(=O)c2ccc3c(Cl)c4c(nc3c2)CCCC4)CC1] | 3.5 |
| A051 | 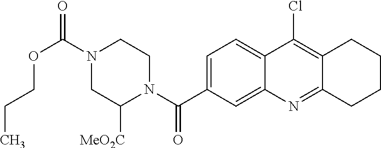 [SMILES: CCCOC(=O)N1CCN(C(=O)c2ccc3c(Cl)c4c(nc3c2)CCCC4)C(C(=O)OC)C1] | 0.65 |
| A052 | 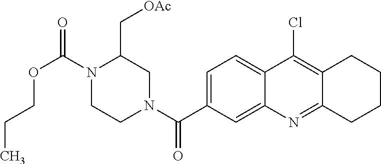 [SMILES: CCCOC(=O)N1CCN(C(=O)c2ccc3c(Cl)c4c(nc3c2)CCCC4)CC1COC(C)=O] | 1.8 |
| A053 | 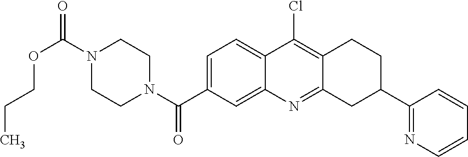 [SMILES: CCCOC(=O)N1CCN(C(=O)c2ccc3c(Cl)c4c(nc3c2)CC(c2ccccn2)CC4)CC1] | 0.24 |
| C008 | 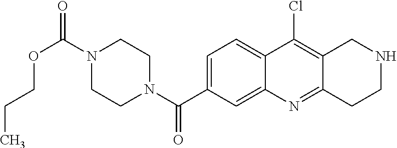 [SMILES: CCCOC(=O)N1CCN(C(=O)c2ccc3c(Cl)c4c(nc3c2)CCNC4)CC1] | 4.6 |
| A054 | 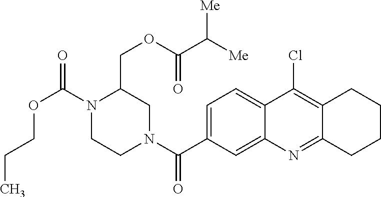 [SMILES: CCCOC(=O)N1CCN(C(=O)c2ccc3c(Cl)c4c(nc3c2)CCCC4)CC1COC(=O)C(C)C] | 5.2 |
| A055 | 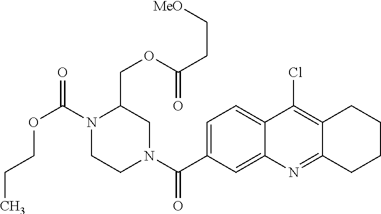 [SMILES: CCCOC(=O)N1CCN(C(=O)c2ccc3c(Cl)c4c(nc3c2)CCCC4)CC1COC(=O)CCOC] | 2.4 |
| A056 | 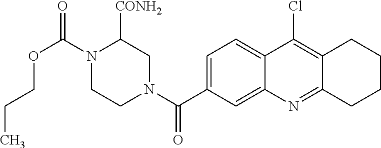 [SMILES: CCCOC(=O)N1CCN(C(=O)c2ccc3c(Cl)c4c(nc3c2)CCCC4)CC1C(N)=O] | 1.7 |
| A057 | 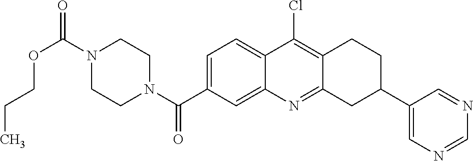 [SMILES: CCCOC(=O)N1CCN(C(=O)c2ccc3c(Cl)c4c(nc3c2)CC(c2cncnc2)CC4)CC1] | 0.31 |
| A058 | 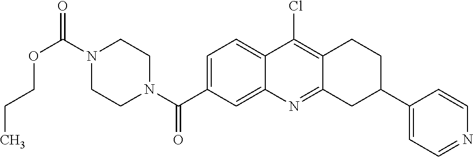 [SMILES: CCCOC(=O)N1CCN(C(=O)c2ccc3c(Cl)c4c(nc3c2)CC(c2ccncc2)CC4)CC1] | 0.19 |
| A059 | 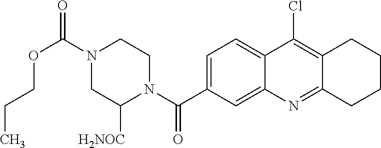 [SMILES: CCCOC(=O)N1CCN(C(=O)c2ccc3c(Cl)c4c(nc3c2)CCCC4)C(C(N)=O)C1] | 2.3 |
| A060 | 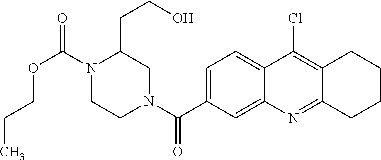 [SMILES: CCCOC(=O)N1CCN(C(=O)c2ccc3c(Cl)c4c(nc3c2)CCCC4)CC1CCO] | 2.7 |
| B003 | 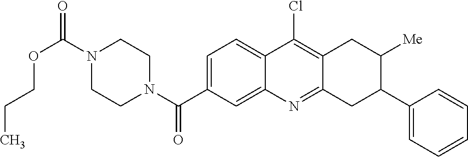 [SMILES: CCCOC(=O)N1CCN(C(=O)c2ccc3c(Cl)c4c(nc3c2)CC(c2ccccc2)C(C)C4)CC1] | 0.38 |
| B004 | 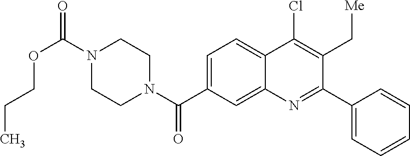 [SMILES: CCCOC(=O)N1CCN(C(=O)c2ccc3c(Cl)c(CC)c(-c4ccccc4)nc3c2)CC1] | 1.4 |
| A061 | 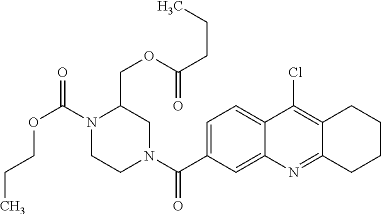 [SMILES: CCCOC(=O)N1CCN(C(=O)c2ccc3c(Cl)c4c(nc3c2)CCCC4)CC1COC(=O)CCC] | 4.1 |
| A062 | 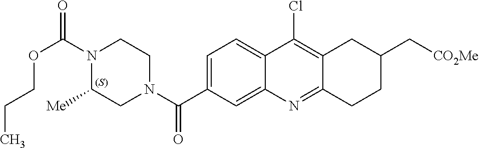 | 0.21 |
| A063 | 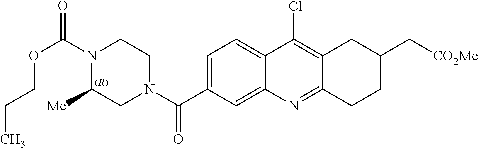 | 0.25 |
| A064 | 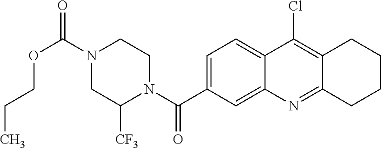 [SMILES: CCCOC(=O)N1CCN(C(=O)c2ccc3c(Cl)c4c(nc3c2)CCCC4)C(C(F)(F)F)C1] | 0.7 |
| A065 | 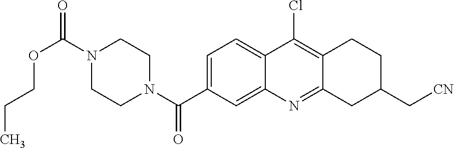 [SMILES: CCCOC(=O)N1CCN(C(=O)c2ccc3c(Cl)c4c(nc3c2)CC(CC#N)CC4)CC1] | 0.19 |
| A066 | 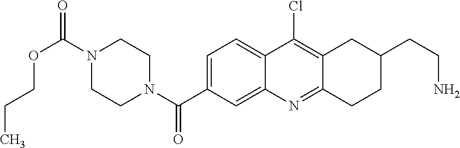 [SMILES: CCCOC(=O)N1CCN(C(=O)c2ccc3c(Cl)c4c(nc3c2)CCC(CCN)C4)CC1] | 0.3 |
| A067 | 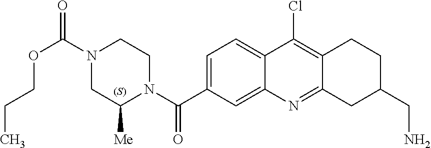 | 0.16 |
| A068 | 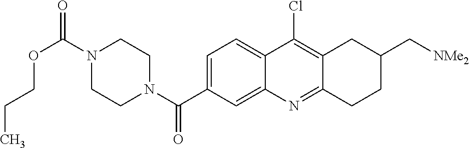 [SMILES: CCCOC(=O)N1CCN(C(=O)c2ccc3c(Cl)c4c(nc3c2)CCC(CN(C)C)C4)CC1] | 1.2 |
| A069 | 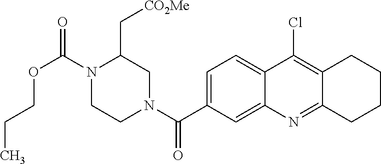 [SMILES: CCCOC(=O)N1CCN(C(=O)c2ccc3c(Cl)c4c(nc3c2)CCCC4)CC1CC(=O)OC] | 5.2 |
| A070 | 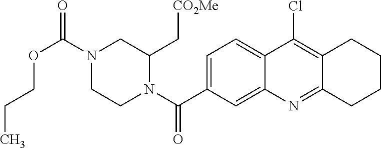 [SMILES: CCCOC(=O)N1CCN(C(=O)c2ccc3c(Cl)c4c(nc3c2)CCCC4)C(CC(=O)OC)C1] | 2.6 |
| A071 | 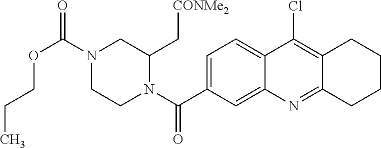 | 4.2 |
| A072 | 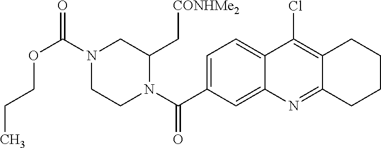 | 4.3 |
| A073 | 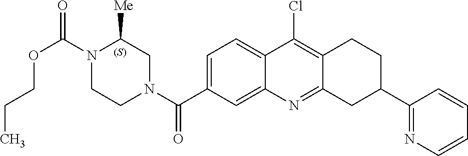 | 0.15 |
| A074 | 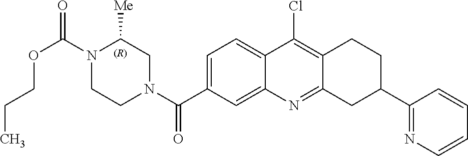 | 0.15 |
| B005 | 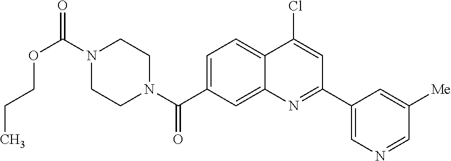 [SMILES: CCCOC(=O)N1CCN(C(=O)c2ccc3c(Cl)cc(-c4cncc(C)c4)nc3c2)CC1] | 2.5 |
| A075 | 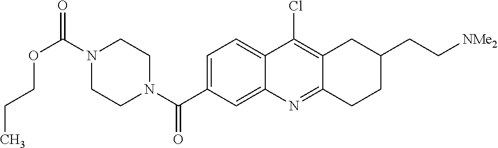 [SMILES: CCCOC(=O)N1CCN(C(=O)c2ccc3c(Cl)c4c(nc3c2)CCC(CCN(C)C)C4)CC1] | 0.21 |
| A076 | 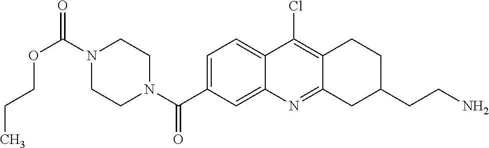 [SMILES: CCCOC(=O)N1CCN(C(=O)c2ccc3c(Cl)c4c(nc3c2)CC(CCN)CC4)CC1] | 0.16 |
| A077 | 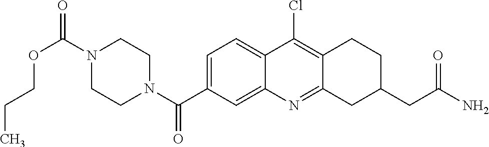 [SMILES: CCCOC(=O)N1CCN(C(=O)c2ccc3c(Cl)c4c(nc3c2)CC(CC(N)=O)CC4)CC1] | 1.2 |
| A078 | 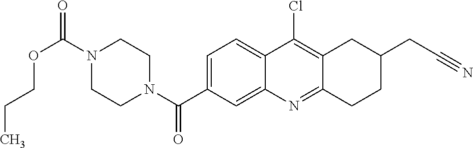 [SMILES: CCCOC(=O)N1CCN(C(=O)c2ccc3c(Cl)c4c(nc3c2)CCC(CC#N)C4)CC1] | 0.28 |
| B006 | 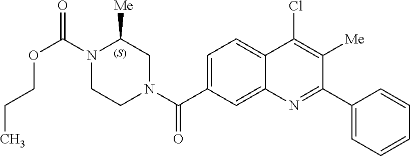 | 0.16 |
| B007 | 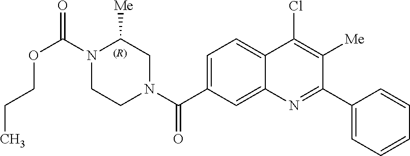 | 0.22 |
| A079 | 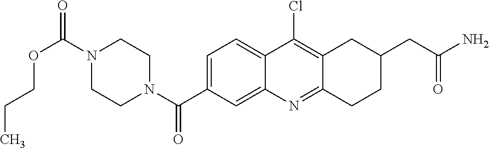 [SMILES: CCCOC(=O)N1CCN(C(=O)c2ccc3c(Cl)c4c(nc3c2)CCC(CC(N)=O)C4)CC1] | 0.43 |
| A080 | 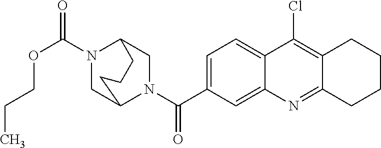 [SMILES: CCCOC(=O)N1CC2CCCC1CN2C(=O)c1ccc2c(Cl)c3c(nc2c1)CCCC3] | 0.17 |
| B008 | 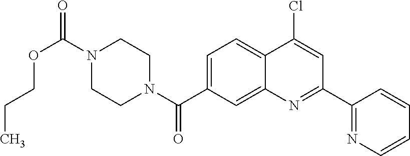 [SMILES: CCCOC(=O)N1CCN(C(=O)c2ccc3c(Cl)cc(-c4ccccn4)nc3c2)CC1] | 1.4 |
| B009 | 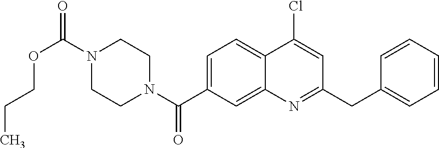 [SMILES: CCCOC(=O)N1CCN(C(=O)c2ccc3c(Cl)cc(Cc4ccccc4)nc3c2)CC1] | 3.3 |
| A081 | 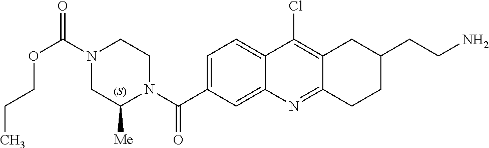 | 0.15 |
| B010 | 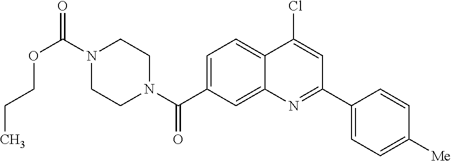 [SMILES: CCCOC(=O)N1CCN(C(=O)c2ccc3c(Cl)cc(-c4ccc(C)cc4)nc3c2)CC1] | 0.50 |
| B011 | 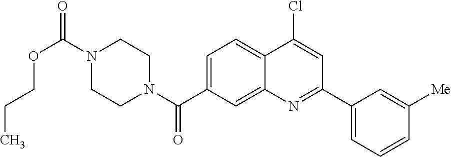 [SMILES: CCCOC(=O)N1CCN(C(=O)c2ccc3c(Cl)cc(-c4cccc(C)c4)nc3c2)CC1] | 0.77 |
| B012 | 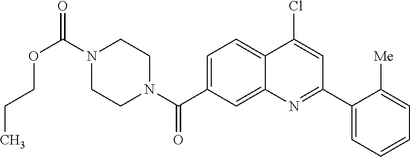 [SMILES: CCCOC(=O)N1CCN(C(=O)c2ccc3c(Cl)cc(-c4ccccc4C)nc3c2)CC1] | 2.9 |
| B013 | 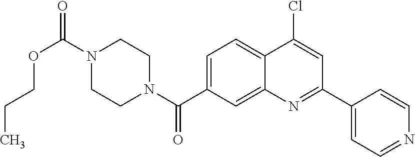 [SMILES: CCCOC(=O)N1CCN(C(=O)c2ccc3c(Cl)cc(-c4ccncc4)nc3c2)CC1] | 0.95 |
| A082 | 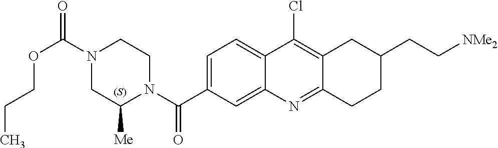 | 0.51 |
| B014 | 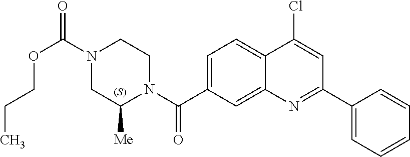 | 0.17 |
| B015 | 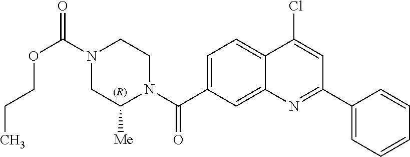 | 0.19 |
| B016 | 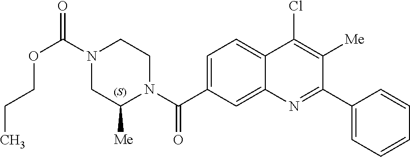 | 0.15 |
| B017 | 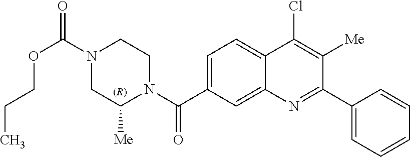 | 0.30 |
| B018 | 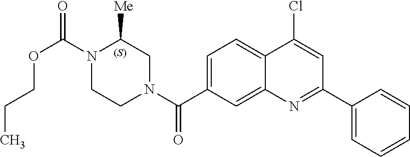 | 0.13 |
| B019 | 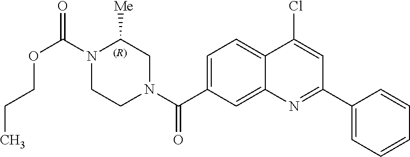 | 0.06 |
| A083 | 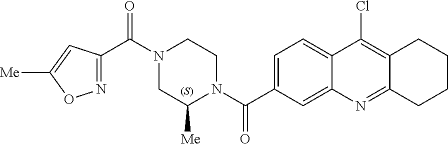 | 0.44 |
| A084 | 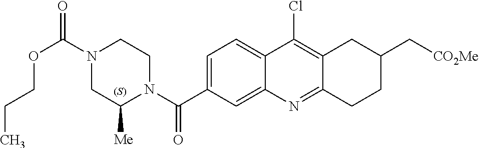 | 0.12 |
| A085 | 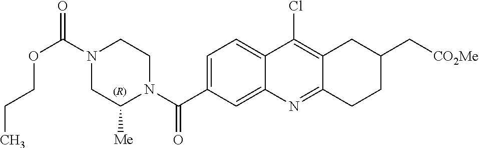 | 0.16 |
| A086 | 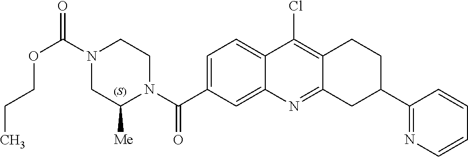 | 0.095 |
| A087 | 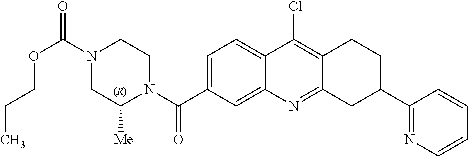 | 0.056 |
| A088 | 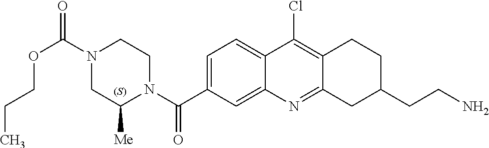 | 0.09 |
| A089 | 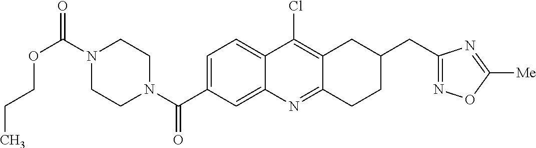 [SMILES: CCCOC(=O)N1CCN(C(=O)c2ccc3c(Cl)c4c(nc3c2)CCC(Cc2noc(C)n2)C4)CC1] | 0.35 |
| A090 | 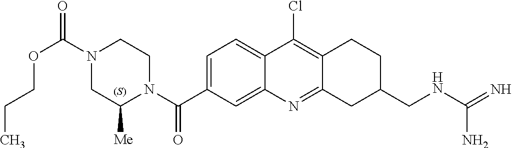 | 0.35 |
| A091 | 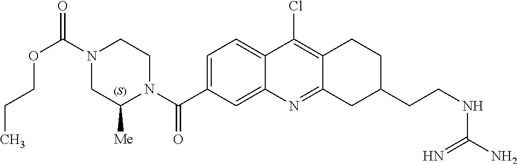 | 0.14 |
| B020 | 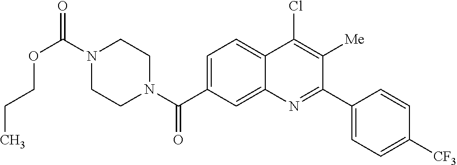 [SMILES: CCCOC(=O)N1CCN(C(=O)c2ccc3c(Cl)c(C)c(-c4ccc(C(F)(F)F)cc4)nc3c2)CC1] | 3.2 |
| B021 | 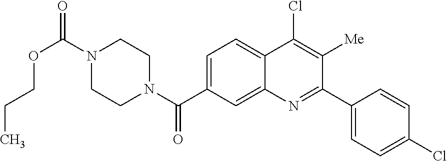 [SMILES: CCCOC(=O)N1CCN(C(=O)c2ccc3c(Cl)c(C)c(-c4ccc(Cl)cc4)nc3c2)CC1] | 1.3 |
| B022 | 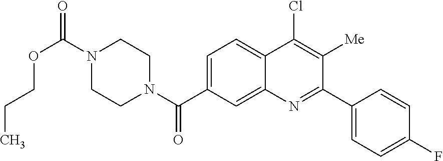 [SMILES: CCCOC(=O)N1CCN(C(=O)c2ccc3c(Cl)c(C)c(-c4ccc(F)cc4)nc3c2)CC1] | 0.85 |
| B023 | 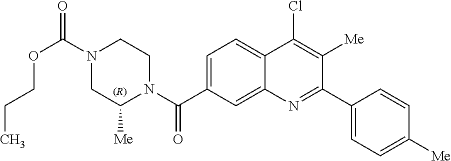 | 0.69 |
| A092 | 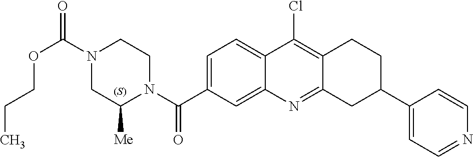 | 0.11 |
| A093 | 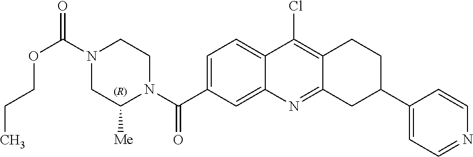 | 0.10 |
| B024 | 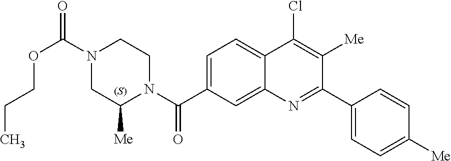 | 0.67 |
| B025 | 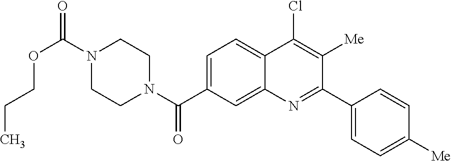 [SMILES: CCCOC(=O)N1CCN(C(=O)c2ccc3c(Cl)c(C)c(-c4ccc(C)cc4)nc3c2)CC1] | 0.67 |
| B026 | 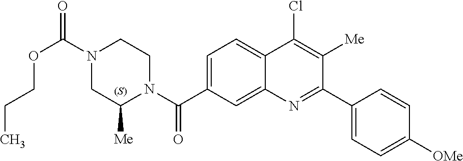 | 0.27 |
| A094 | 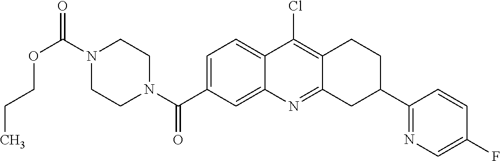 [SMILES: CCCOC(=O)N1CCN(C(=O)c2ccc3c(Cl)c4c(nc3c2)CC(c2ccc(F)cn2)CC4)CC1] | 0.14 |
| A095 | 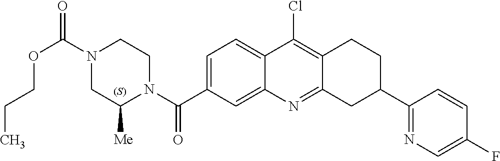 | 0.11 |
| A096 | 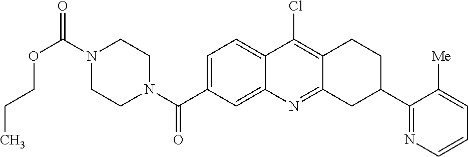 [SMILES: CCCOC(=O)N1CCN(C(=O)c2ccc3c(Cl)c4c(nc3c2)CC(c2ncccc2C)CC4)CC1] | 1.2 |
| B027 | 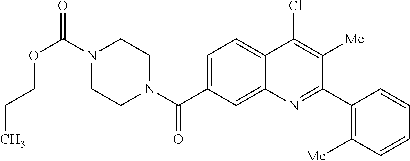 [SMILES: CCCOC(=O)N1CCN(C(=O)c2ccc3c(Cl)c(C)c(-c4ccccc4C)nc3c2)CC1] | 1.2 |
| B028 | 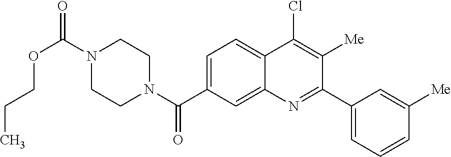 [SMILES: CCCOC(=O)N1CCN(C(=O)c2ccc3c(Cl)c(C)c(-c4cccc(C)c4)nc3c2)CC1] | 1.1 |
| B029 | 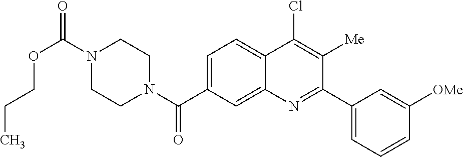 [SMILES: CCCOC(=O)N1CCN(C(=O)c2ccc3c(Cl)c(C)c(-c4cccc(OC)c4)nc3c2)CC1] | 1.4 |
| B030 | 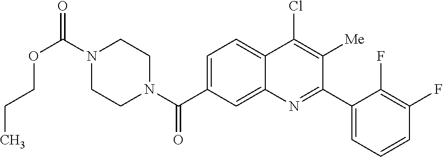 [SMILES: CCCOC(=O)N1CCN(C(=O)c2ccc3c(Cl)c(C)c(-c4cccc(F)c4F)nc3c2)CC1] | 5.8 |
| A097 | 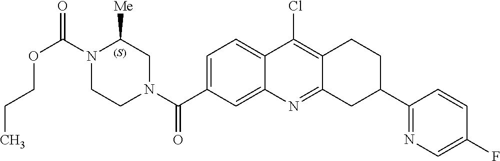 | 0.17 |
| A098 | 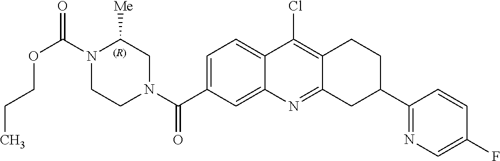 | 0.15 |
| A099 | 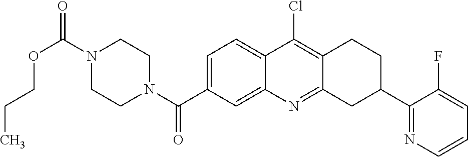 [SMILES: CCCOC(=O)N1CCN(C(=O)c2ccc3c(Cl)c4c(nc3c2)CC(c2ncccc2F)CC4)CC1] | 0.59 |
| A100 | 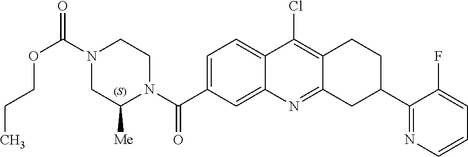 | 0.57 |
| A053 (Ent-1) | 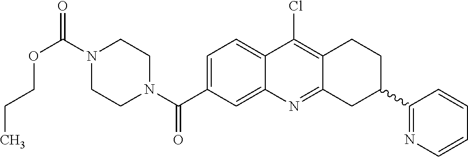 [SMILES: CCCOC(=O)N1CCN(C(=O)c2ccc3c(Cl)c4c(nc3c2)CC(c2ccccn2)CC4)CC1] | 0.12 |
| A053 (Ent-2) | 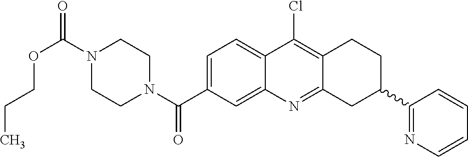 [SMILES: CCCOC(=O)N1CCN(C(=O)c2ccc3c(Cl)c4c(nc3c2)CC(c2ccccn2)CC4)CC1] | 0.24 |
| A101 | 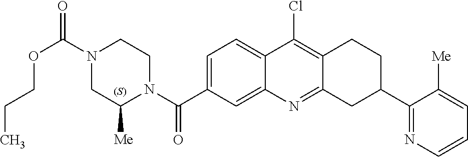 | 1.7 |
| A102 | 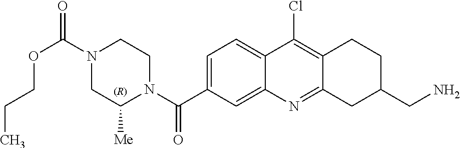 | 0.23 |
| A103 | 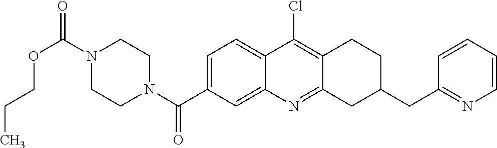 [SMILES: CCCOC(=O)N1CCN(C(=O)c2ccc3c(Cl)c4c(nc3c2)CC(Cc2ccccn2)CC4)CC1] | 0.39 |
| A104 | 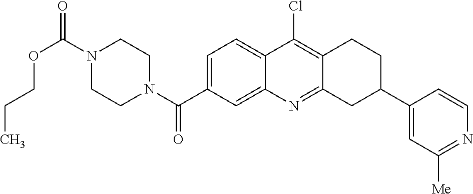 [SMILES: CCCOC(=O)N1CCN(C(=O)c2ccc3c(Cl)c4c(nc3c2)CC(c2ccnc(C)c2)CC4)CC1] | 0.083 |
| A105 | 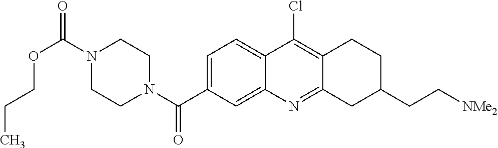 [SMILES: CCCOC(=O)N1CCN(C(=O)c2ccc3c(Cl)c4c(nc3c2)CC(CCN(C)C)CC4)CC1] | 0.09 |
| A106 | 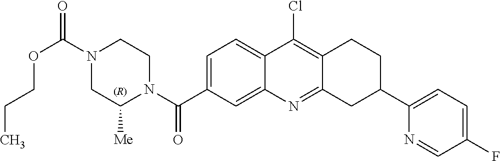 | 0.041 |
| A107 | 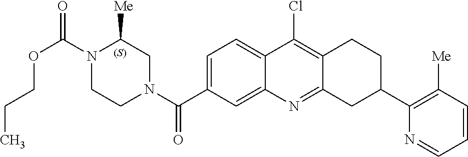 | 0.38 |
| A108 | 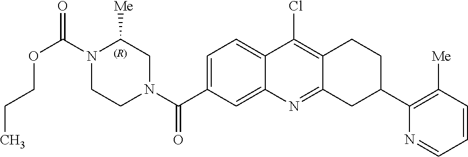 | 0.48 |
| A109 | 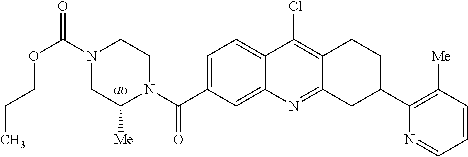 | 0.72 |
| B031 | 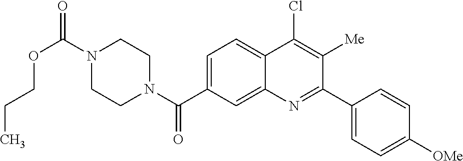 [SMILES: CCCOC(=O)N1CCN(C(=O)c2ccc3c(Cl)c(C)c(-c4ccc(OC)cc4)nc3c2)CC1] | 0.28 |
| A110 | 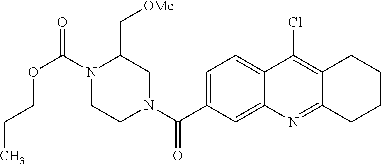 [SMILES: CCCOC(=O)N1CCN(C(=O)c2ccc3c(Cl)c4c(nc3c2)CCCC4)CC1COC] | 0.84 |
| A111 | 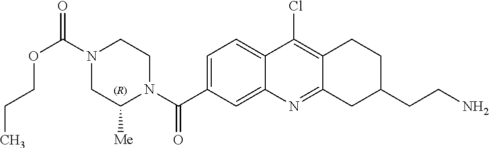 | 0.047 |
| B032 | 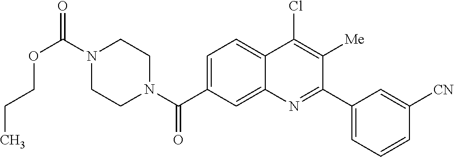 [SMILES: CCCOC(=O)N1CCN(C(=O)c2ccc3c(Cl)c(C)c(-c4cccc(C#N)c4)nc3c2)CC1] | 5.5 |
| B033 | 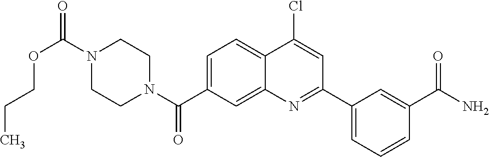 [SMILES: CCCOC(=O)N1CCN(C(=O)c2ccc3c(Cl)cc(-c4cccc(C(N)=O)c4)nc3c2)CC1] | 0.21 |
| A112 | 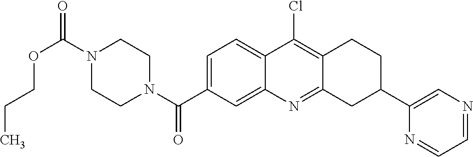 [SMILES: CCCOC(=O)N1CCN(C(=O)c2ccc3c(Cl)c4c(nc3c2)CC(c2cnccn2)CC4)CC1] | 0.14 |
| A113 | 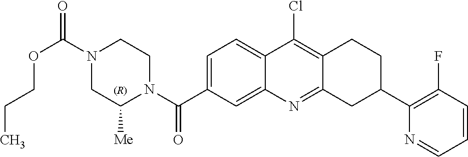 | 0.34 |
| A114 | 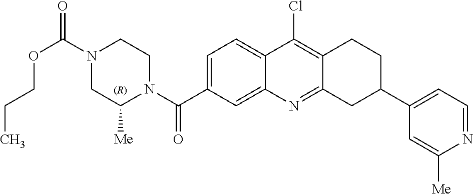 | 0.11 |
| A115 | 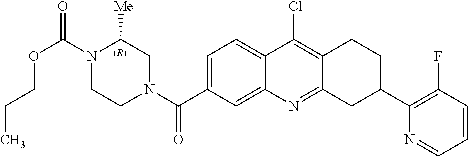 | 0.27 |
| A116 | 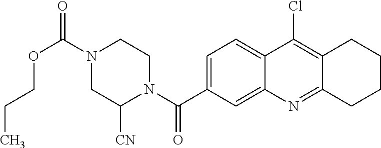 [SMILES: CCCOC(=O)N1CCN(C(=O)c2ccc3c(Cl)c4c(nc3c2)CCCC4)C(C#N)C1] | 0.21 |
| A117 | 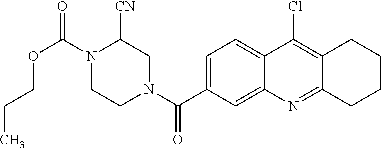 [SMILES: CCCOC(=O)N1CCN(C(=O)c2ccc3c(Cl)c4c(nc3c2)CCCC4)CC1C#N] | 0.12 |
| A118 | 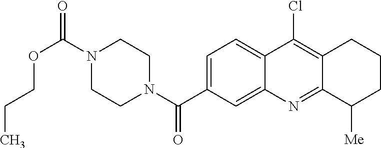 [SMILES: CCCOC(=O)N1CCN(C(=O)c2ccc3c(Cl)c4c(nc3c2)C(C)CCC4)CC1] | 0.18 |
| A119 | 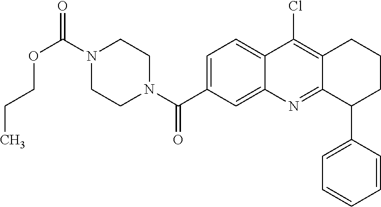 [SMILES: CCCOC(=O)N1CCN(C(=O)c2ccc3c(Cl)c4c(nc3c2)C(c2ccccc2)CCC4)CC1] | 1.9 |
| A120 | 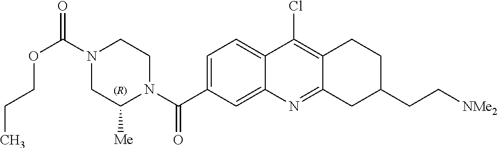 | 0.12 |
| A121 | 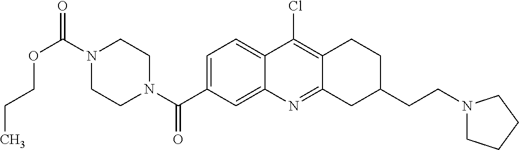 [SMILES: CCCOC(=O)N1CCN(C(=O)c2ccc3c(Cl)c4c(nc3c2)CC(CCN2CCCC2)CC4)CC1] | 0.22 |
| B034 | 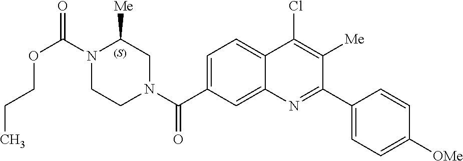 | 0.12 |
| A122 | 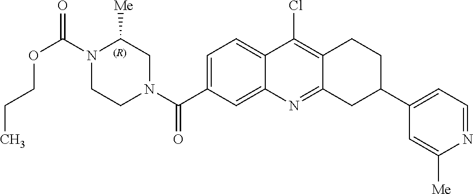 | 0.12 |
| A123 | 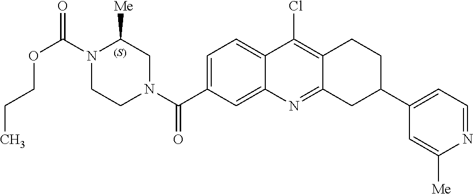 | 0.14 |
| A124 | 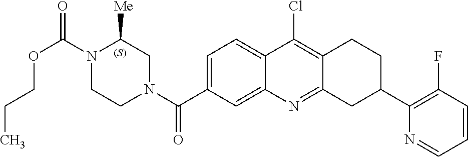 | 0.34 |
| B035 | 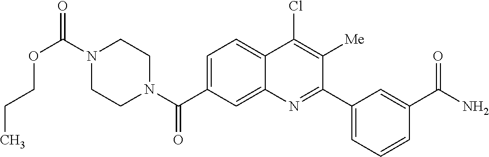 [SMILES: CCCOC(=O)N1CCN(C(=O)c2ccc3c(Cl)c(C)c(-c4cccc(C(N)=O)c4)nc3c2)CC1] | 1.0 |
| A125 | 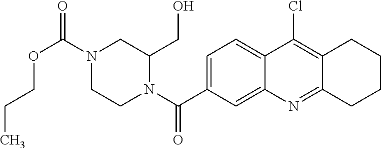 [SMILES: CCCOC(=O)N1CCN(C(=O)c2ccc3c(Cl)c4c(nc3c2)CCCC4)C(CO)C1] | 0.25 |
| A126 | 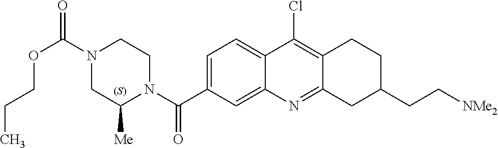 | 0.11 |
| B036 | 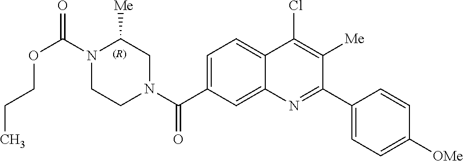 | 0.15 |
| B037 | 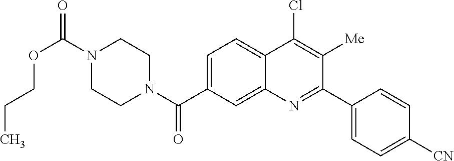 [SMILES: CCCOC(=O)N1CCN(C(=O)c2ccc3c(Cl)c(C)c(-c4ccc(C#N)cc4)nc3c2)CC1] | 1.8 |
| A127 | 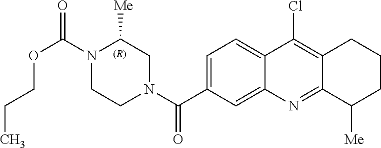 | 0.11 |
| A128 | 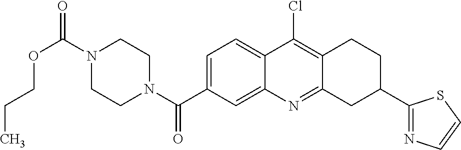 [SMILES: CCCOC(=O)N1CCN(C(=O)c2ccc3c(Cl)c4c(nc3c2)CC(c2nccs2)CC4)CC1] | 0.10 |
| A129 | 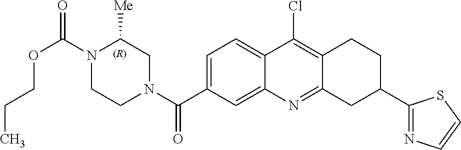 | 0.094 |
| A130 | 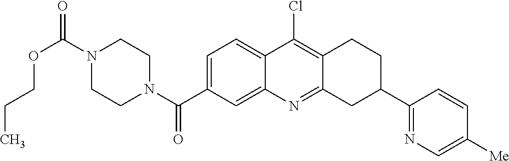 [SMILES: CCCOC(=O)N1CCN(C(=O)c2ccc3c(Cl)c4c(nc3c2)CC(c2ccc(C)cn2)CC4)CC1] | 0.13 |
| A131 | 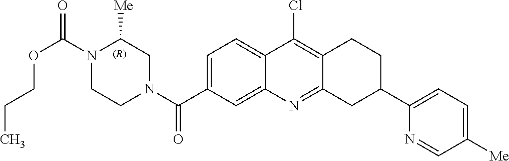 | 0.11 |
| B038 | 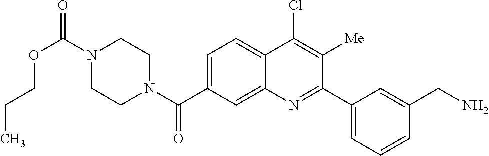 [SMILES: CCCOC(=O)N1CCN(C(=O)c2ccc3c(Cl)c(C)c(-c4cccc(CN)c4)nc3c2)CC1] | 0.099 |
| B039 | 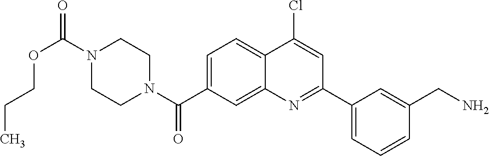 [SMILES: CCCOC(=O)N1CCN(C(=O)c2ccc3c(Cl)cc(-c4cccc(CN)c4)nc3c2)CC1] | 0.091 |
| B040 | 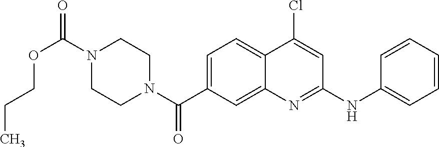 [SMILES: CCCOC(=O)N1CCN(C(=O)c2ccc3c(Cl)cc(Nc4ccccc4)nc3c2)CC1] | 3.2 |
| A132 | 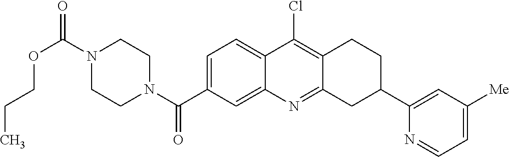 [SMILES: CCCOC(=O)N1CCN(C(=O)c2ccc3c(Cl)c4c(nc3c2)CC(c2cc(C)ccn2)CC4)CC1] | 0.21 |
| A133 | 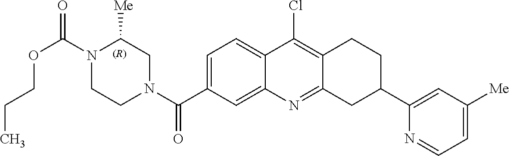 | 0.12 |
| A134 | 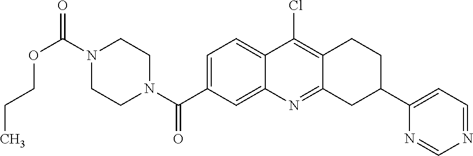 [SMILES: CCCOC(=O)N1CCN(C(=O)c2ccc3c(Cl)c4c(nc3c2)CC(c2ccncn2)CC4)CC1] | 0.14 |
| B041 | 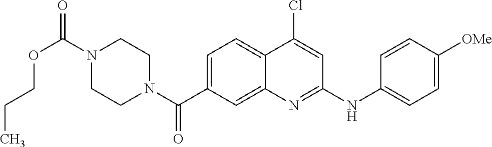 [SMILES: CCCOC(=O)N1CCN(C(=O)c2ccc3c(Cl)cc(Nc4ccc(OC)cc4)nc3c2)CC1] | 1.2 |
| B042 | 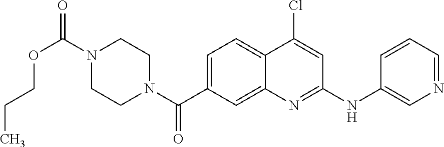 [SMILES: CCCOC(=O)N1CCN(C(=O)c2ccc3c(Cl)cc(Nc4cccnc4)nc3c2)CC1] | 3.1 |
| B043 | 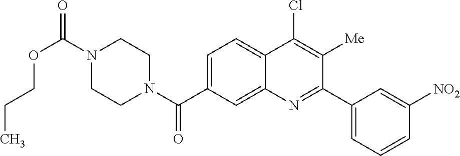 [SMILES: CCCOC(=O)N1CCN(C(=O)c2ccc3c(Cl)c(C)c(-c4cccc([N+](=O)[O-])c4)nc3c2)CC1] | 2.4 |
| B044 | 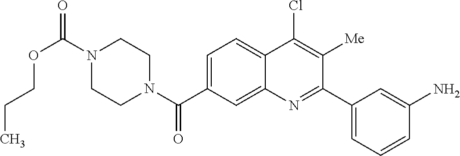 [SMILES: CCCOC(=O)N1CCN(C(=O)c2ccc3c(Cl)c(C)c(-c4cccc(N)c4)nc3c2)CC1] | 0.045 |
| B045 | 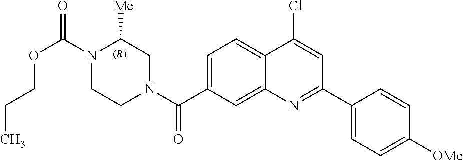 | 0.44 |
| B046 | 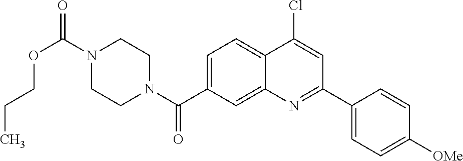 [SMILES: CCCOC(=O)N1CCN(C(=O)c2ccc3c(Cl)cc(-c4ccc(OC)cc4)nc3c2)CC1] | 0.26 |
| A135 | 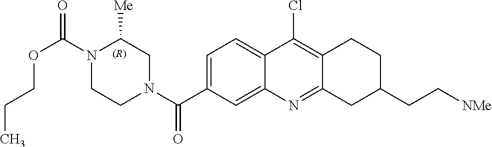 | 0.075 |
| A136 | 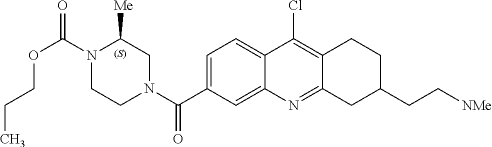 | 0.069 |
| B047 | 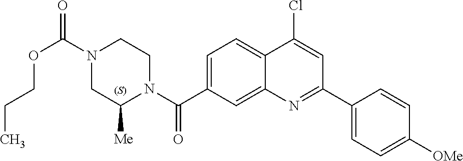 | 0.23 |
| B048 | 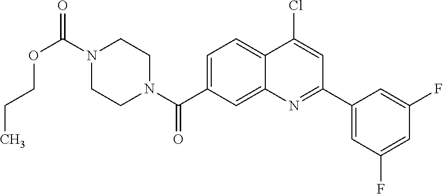 [SMILES: CCCOC(=O)N1CCN(C(=O)c2ccc3c(Cl)cc(-c4cc(F)cc(F)c4)nc3c2)CC1] | 3.0 |
| A137 | 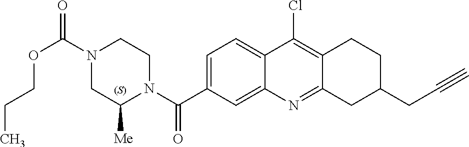 | 0.29 |
| A138 | 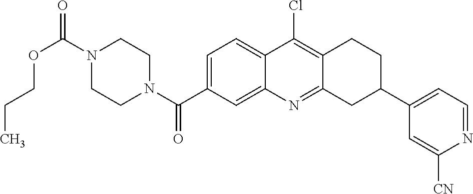 [SMILES: CCCOC(=O)N1CCN(C(=O)c2ccc3c(Cl)c4c(nc3c2)CC(c2ccnc(C#N)c2)CC4)CC1] | 0.68 |
| A139 | 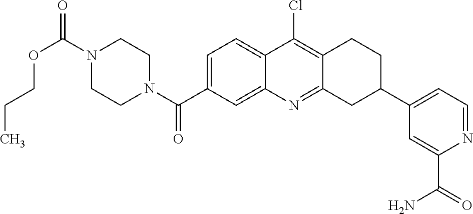 [SMILES: CCCOC(=O)N1CCN(C(=O)c2ccc3c(Cl)c4c(nc3c2)CC(c2ccnc(C(N)=O)c2)CC4)CC1] | 0.35 |
| B049 | 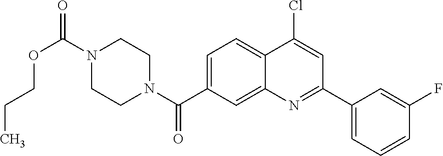 [SMILES: CCCOC(=O)N1CCN(C(=O)c2ccc3c(Cl)cc(-c4cccc(F)c4)nc3c2)CC1] | 1.0 |
| B050 | 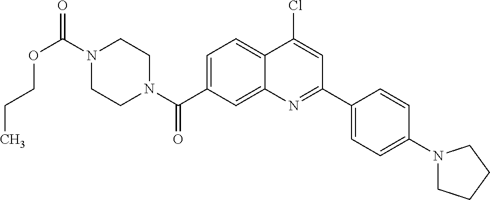 [SMILES: CCCOC(=O)N1CCN(C(=O)c2ccc3c(Cl)cc(-c4ccc(N5CCCC5)cc4)nc3c2)CC1] | 0.79 |
| B051 | 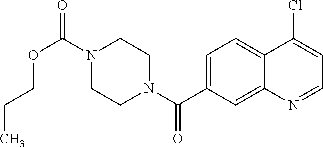 [SMILES: CCCOC(=O)N1CCN(C(=O)c2ccc3c(Cl)ccnc3c2)CC1] | 3.2 |
| B052 | 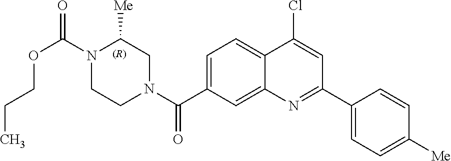 | 0.77 |
| B053 | 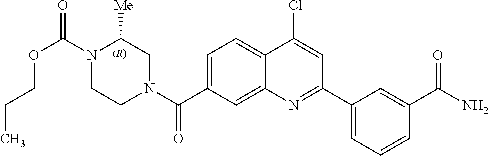 | 0.18 |
| B054 | 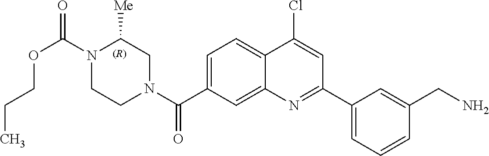 | 0.10 |
| A140 | 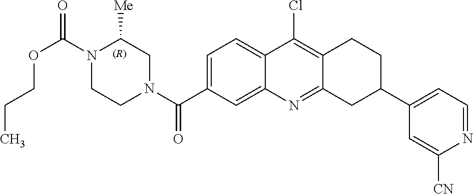 | 0.42 |
| A141 | 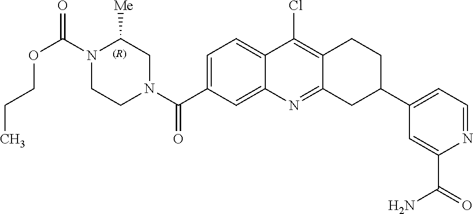 | 0.23 |
| A142 | 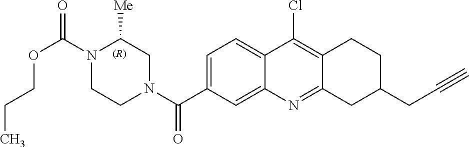 | 0.42 |
| A143 | 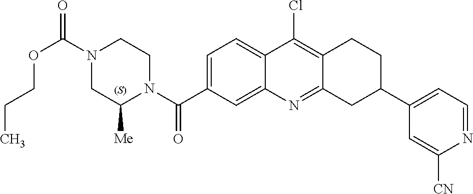 | 0.46 |
| A144 | 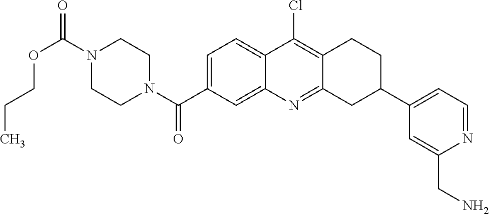 [SMILES: CCCOC(=O)N1CCN(C(=O)c2ccc3c(Cl)c4c(nc3c2)CC(c2ccnc(CN)c2)CC4)CC1] | 0.51 |
| A145 | 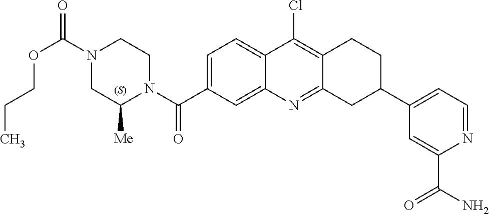 | 0.22 |
| B055 | 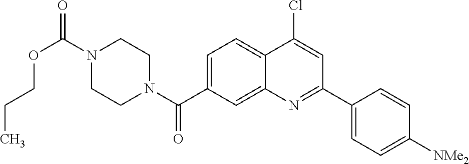 [SMILES: CCCOC(=O)N1CCN(C(=O)c2ccc3c(Cl)cc(-c4ccc(N(C)C)cc4)nc3c2)CC1] | 0.35 |
| B056 | 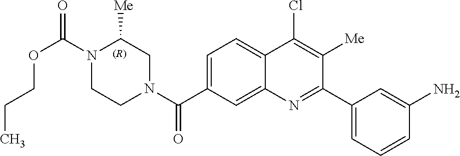 | 0.12 |
| B057 | 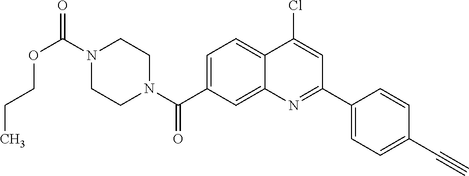 [SMILES: C#Cc1ccc(-c2cc(Cl)c3ccc(C(=O)N4CCN(C(=O)OCCC)CC4)cc3n2)cc1] | 0.93 |
| B058 | 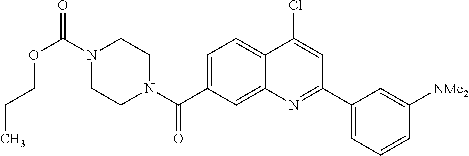 [SMILES: CCCOC(=O)N1CCN(C(=O)c2ccc3c(Cl)cc(-c4cccc(N(C)C)c4)nc3c2)CC1] | 1.47 |
| B059 | 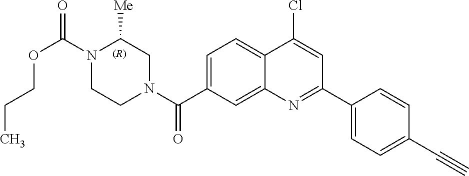 | 1.00 |
| B060 | 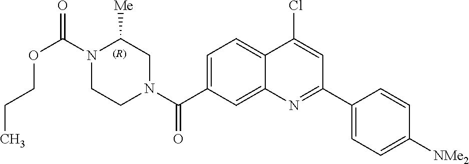 | 0.42 |
| B061 | 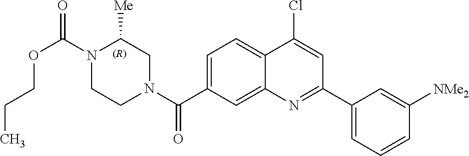 | 0.87 |
| B062 | 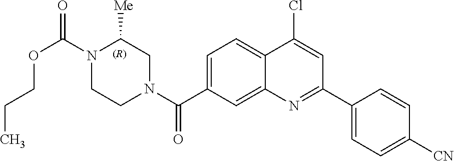 | 0.34 |
| B063 | 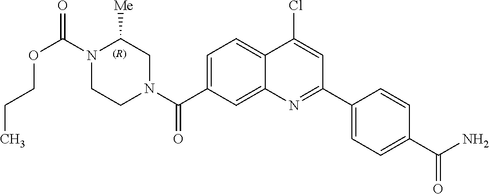 | 0.08 |
| B064 | 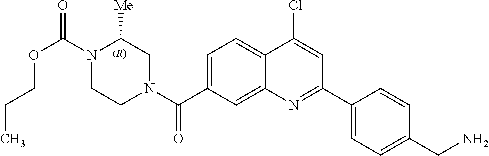 | 0.057 |
| B065 | 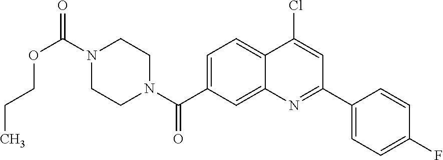 [SMILES: CCCOC(=O)N1CCN(C(=O)c2ccc3c(Cl)cc(-c4ccc(F)cc4)nc3c2)CC1] | 0.56 |
| B066 | 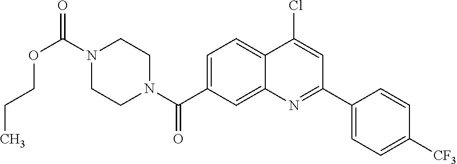 [SMILES: CCCOC(=O)N1CCN(C(=O)c2ccc3c(Cl)cc(-c4ccc(C(F)(F)F)cc4)nc3c2)CC1] | 3.7 |
| A146 | 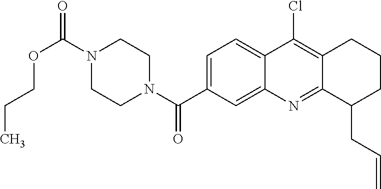 [SMILES: C=CCC1CCCc2c1nc1cc(C(=O)N3CCN(C(=O)OCCC)CC3)ccc1c2Cl] | 0.56 |
| A147 | 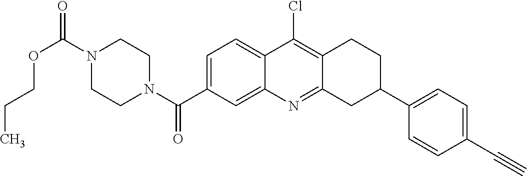 [SMILES: C#Cc1ccc(C2CCc3c(nc4cc(C(=O)N5CCN(C(=O)OCCC)CC5)ccc4c3Cl)C2)cc1] | 0.41 |
| A148 | 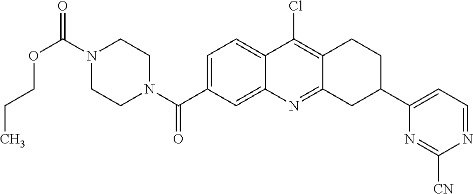 [SMILES: CCCOC(=O)N1CCN(C(=O)c2ccc3c(Cl)c4c(nc3c2)CC(c2ccnc(C#N)n2)CC4)CC1] | 0.30 |
| A149 | 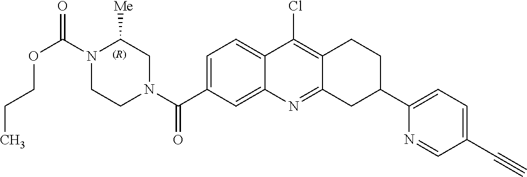 | 0.26 |
| A150 | 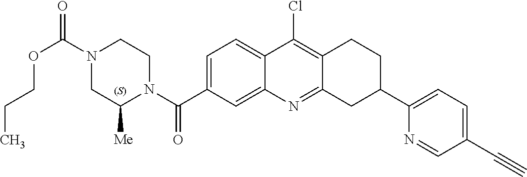 | 0.26 |
| A151 | 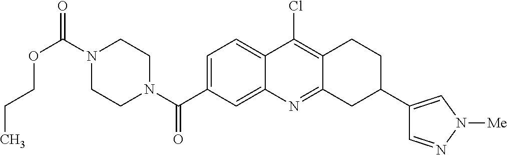 [SMILES: CCCOC(=O)N1CCN(C(=O)c2ccc3c(Cl)c4c(nc3c2)CC(c2cnn(C)c2)CC4)CC1] | 0.046 |
| B067 | 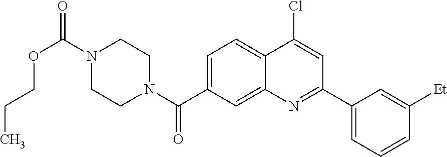 [SMILES: CCCOC(=O)N1CCN(C(=O)c2ccc3c(Cl)cc(-c4cccc(CC)c4)nc3c2)CC1] | 2.1 |
| B068 | 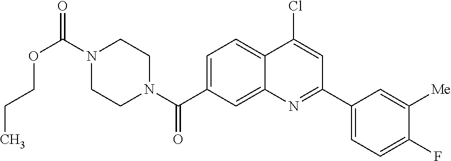 [SMILES: CCCOC(=O)N1CCN(C(=O)c2ccc3c(Cl)cc(-c4ccc(F)c(C)c4)nc3c2)CC1] | 1.3 |
| B069 | 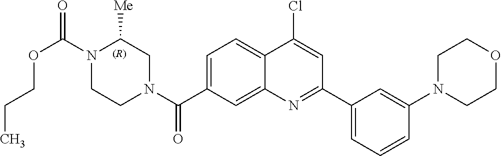 | 0.24 |
| B070 | 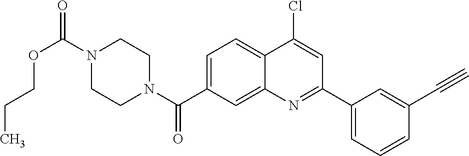 [SMILES: C#Cc1cccc(-c2cc(Cl)c3ccc(C(=O)N4CCN(C(=O)OCCC)CC4)cc3n2)c1] | 2.1 |
| B071 | 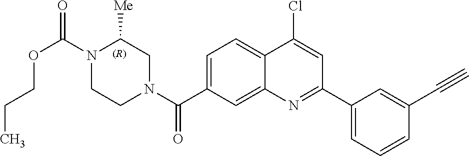 | 1.3 |
| B072 | 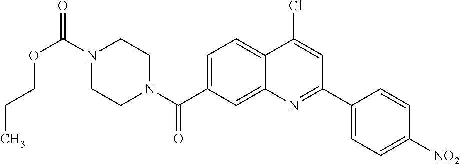 [SMILES: CCCOC(=O)N1CCN(C(=O)c2ccc3c(Cl)cc(-c4ccc([N+](=O)[O-])cc4)nc3c2)CC1] | 1.4 |
| B073 | 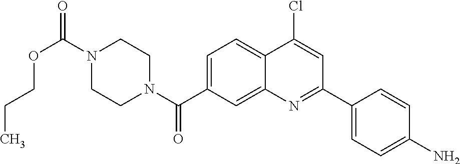 [SMILES: CCCOC(=O)N1CCN(C(=O)c2ccc3c(Cl)cc(-c4ccc(N)cc4)nc3c2)CC1] | 0.090 |
| B074 | 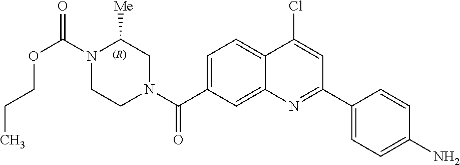 | 0.13 |
| B075 | 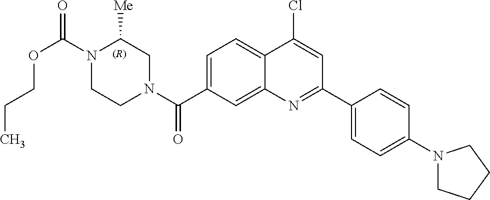 | 0.44 |
| B076 | 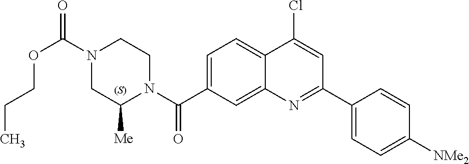 | 0.19 |
| B077 | 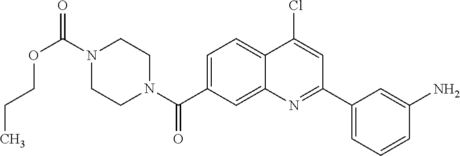 [SMILES: CCCOC(=O)N1CCN(C(=O)c2ccc3c(Cl)cc(-c4cccc(N)c4)nc3c2)CC1] | 0.12 |
| B078 | 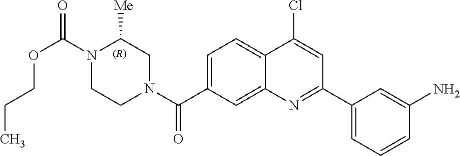 | 0.13 |
| B079 | 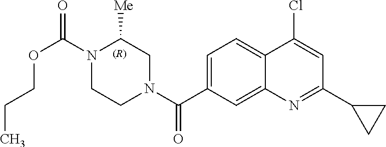 | 0.060 |
| B080 | 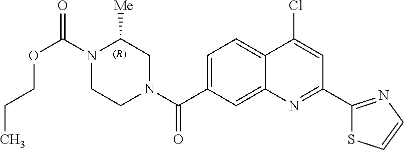 | 10 |
| B081 | 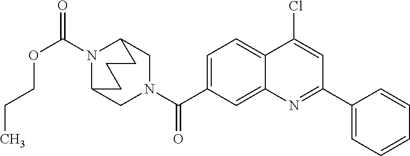 [SMILES: CCCOC(=O)N1C2CCCC1CN(C(=O)c1ccc3c(Cl)cc(-c4ccccc4)nc3c1)C2] | 0.47 |
| B082 | 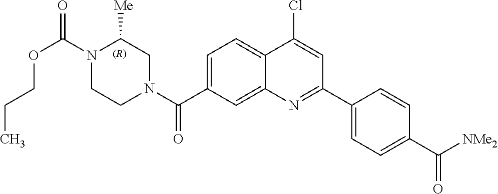 | 0.69 |
| B083 | 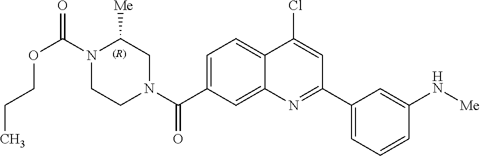 | 0.058 |
| B084 | 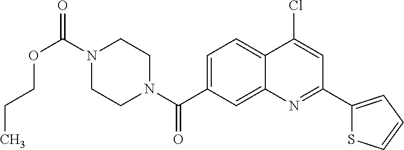 [SMILES: CCCOC(=O)N1CCN(C(=O)c2ccc3c(Cl)cc(-c4cccs4)nc3c2)CC1] | 0.54 |
| B085 | 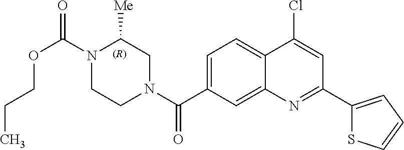 | 0.21 |
| B086 | 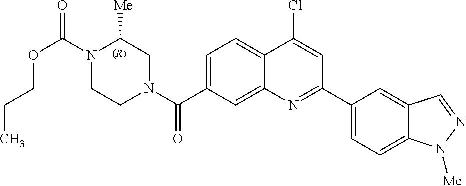 | 0.011 |
| B087 | 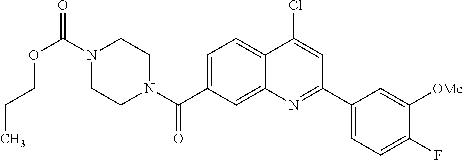 [SMILES: CCCOC(=O)N1CCN(C(=O)c2ccc3c(Cl)cc(-c4ccc(F)c(OC)c4)nc3c2)CC1] | 0.93 |
| B088 | 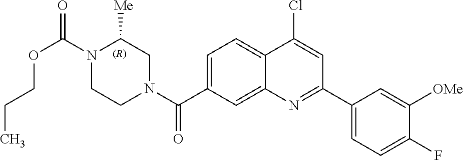 | 0.014 |
| B089 | 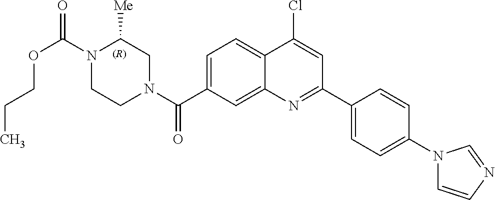 | 0.065 |
| B090 | 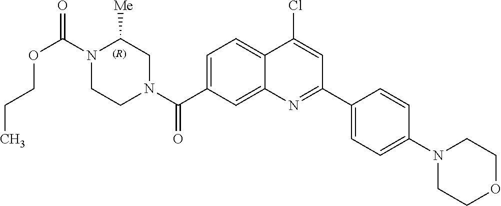 | 0.077 |
| B091 | 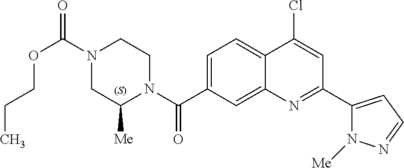 | 7.5 |
| B092 | 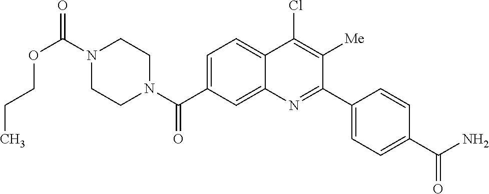 [SMILES: CCCOC(=O)N1CCN(C(=O)c2ccc3c(Cl)c(C)c(-c4ccc(C(N)=O)cc4)nc3c2)CC1] | 0.25 |
| B093 | 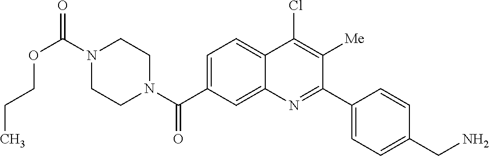 [SMILES: CCCOC(=O)N1CCN(C(=O)c2ccc3c(Cl)c(C)c(-c4ccc(CN)cc4)nc3c2)CC1] | 0.042 |
| B094 | 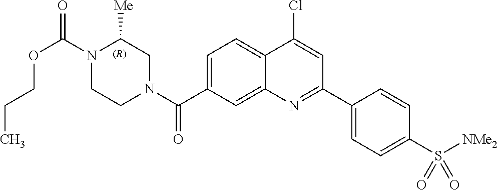 | 0.61 |
| B095 | 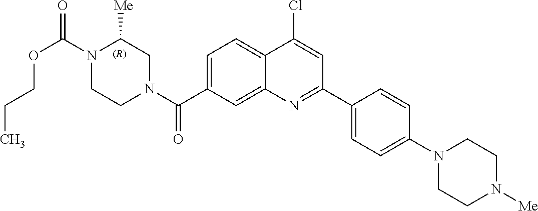 | 0.056 |
| B096 | 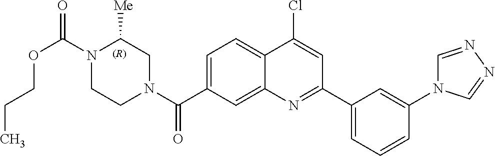 | 0.31 |
| B097 | 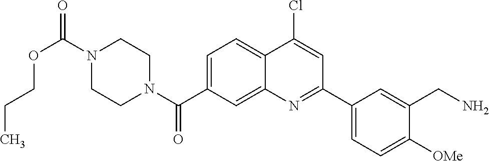 [SMILES: CCCOC(=O)N1CCN(C(=O)c2ccc3c(Cl)cc(-c4ccc(OC)c(CN)c4)nc3c2)CC1] | 0.11 |
| B098 | 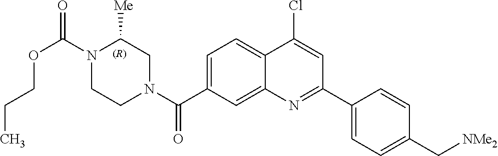 | 0.13 |
| B099 | 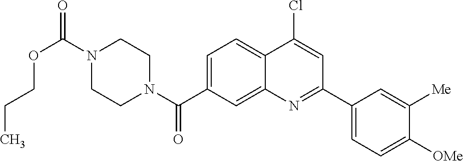 [SMILES: CCCOC(=O)N1CCN(C(=O)c2ccc3c(Cl)cc(-c4ccc(OC)c(C)c4)nc3c2)CC1] | 0.35 |
| B100 | 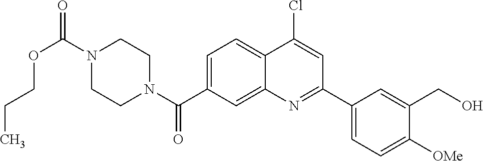 [SMILES: CCCOC(=O)N1CCN(C(=O)c2ccc3c(Cl)cc(-c4ccc(OC)c(CO)c4)nc3c2)CC1] | 0.11 |
| B101 | 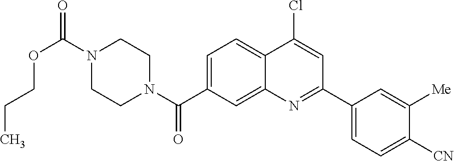 [SMILES: CCCOC(=O)N1CCN(C(=O)c2ccc3c(Cl)cc(-c4ccc(C#N)c(C)c4)nc3c2)CC1] | 1.8 |
| B102 | 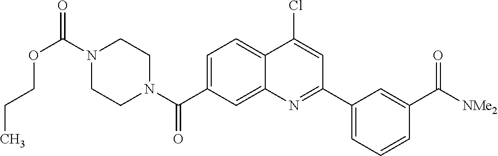 [SMILES: CCCOC(=O)N1CCN(C(=O)c2ccc3c(Cl)cc(-c4cccc(C(=O)N(C)C)c4)nc3c2)CC1] | 0.53 |
| B103 | 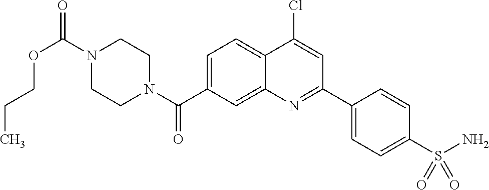 [SMILES: CCCOC(=O)N1CCN(C(=O)c2ccc3c(Cl)cc(-c4ccc(S(N)(=O)=O)cc4)nc3c2)CC1] | 0.13 |
| B104 | 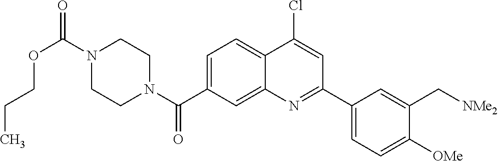 [SMILES: CCCOC(=O)N1CCN(C(=O)c2ccc3c(Cl)cc(-c4ccc(OC)c(CN(C)C)c4)nc3c2)CC1] | 0.12 |
| B105 | 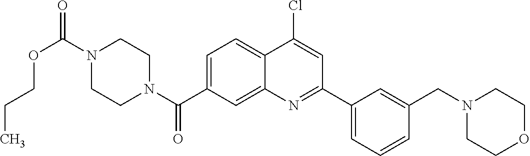 [SMILES: CCCOC(=O)N1CCN(C(=O)c2ccc3c(Cl)cc(-c4cccc(CN5CCOCC5)c4)nc3c2)CC1] | 0.86 |
| B106 | 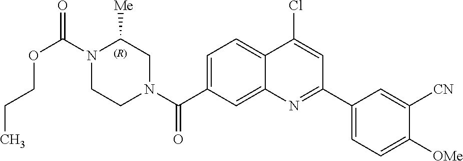 | 0.56 |
| B107 | 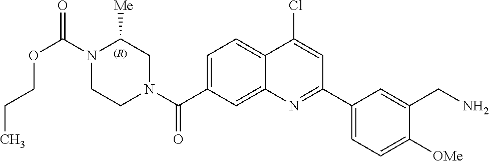 | 0.076 |
| B108 | 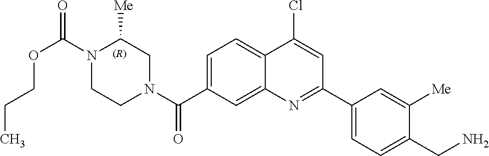 | 0.12 |
| B109 | 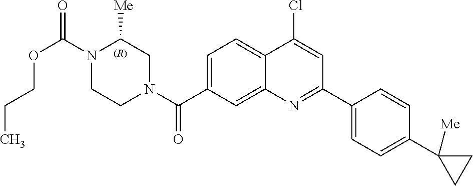 | 0.38 |
| B110 | 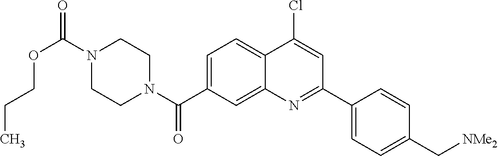 [SMILES: CCCOC(=O)N1CCN(C(=O)c2ccc3c(Cl)cc(-c4ccc(CN(C)C)cc4)nc3c2)CC1] | 0.16 |
| B111 | 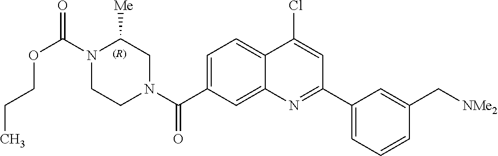 | 0.22 |
| B112 | 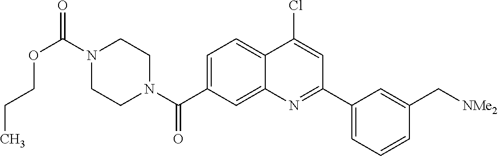 [SMILES: CCCOC(=O)N1CCN(C(=O)c2ccc3c(Cl)cc(-c4cccc(CN(C)C)c4)nc3c2)CC1] | 0.14 |
| B113 | 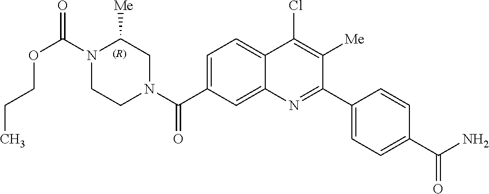 | 0.092 |
| B114 | 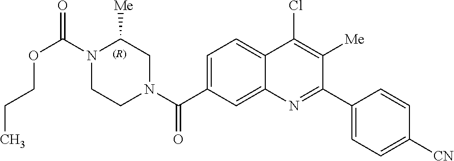 | 0.42 |
| B115 | 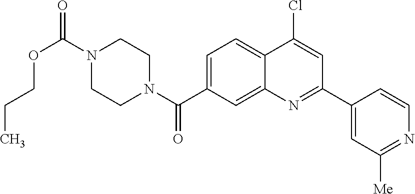 [SMILES: CCCOC(=O)N1CCN(C(=O)c2ccc3c(Cl)cc(-c4ccnc(C)c4)nc3c2)CC1] | 0.78 |
| B116 | 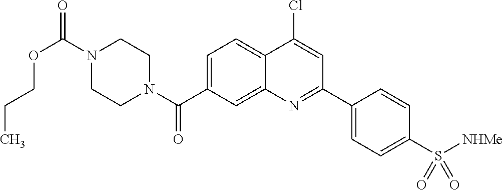 [SMILES: CCCOC(=O)N1CCN(C(=O)c2ccc3c(Cl)cc(-c4ccc(S(=O)(=O)NC)cc4)nc3c2)CC1] | 0.43 |
| B117 | 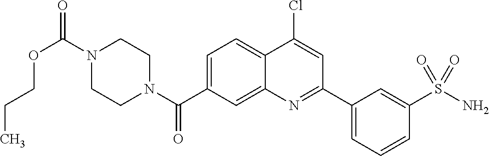 [SMILES: CCCOC(=O)N1CCN(C(=O)c2ccc3c(Cl)cc(-c4cccc(S(N)(=O)=O)c4)nc3c2)CC1] | 0.92 |
| B118 | 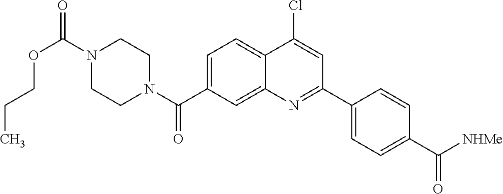 [SMILES: CCCOC(=O)N1CCN(C(=O)c2ccc3c(Cl)cc(-c4ccc(C(=O)NC)cc4)nc3c2)CC1] | 0.25 |
| B119 | 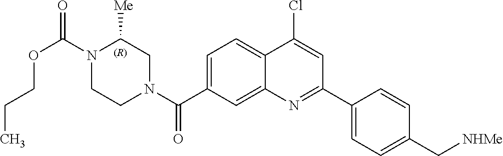 | 0.042 |
| B120 | 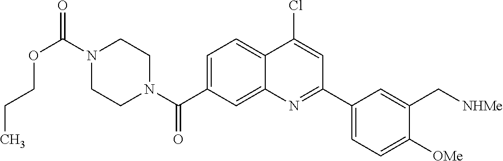 [SMILES: CCCOC(=O)N1CCN(C(=O)c2ccc3c(Cl)cc(-c4ccc(OC)c(CNC)c4)nc3c2)CC1] | 0.035 |
| B121 | 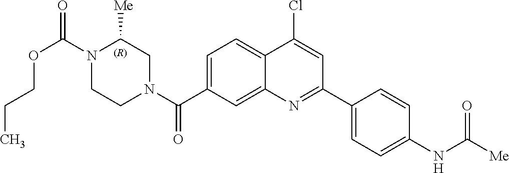 | 0.055 |
| B122 | 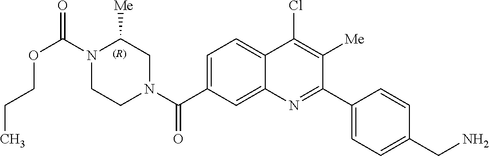 | 0.070 |
| B123 | 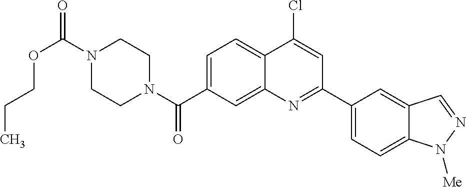 [SMILES: CCCOC(=O)N1CCN(C(=O)c2ccc3c(Cl)cc(-c4ccc5c(cnn5C)c4)nc3c2)CC1] | 0.15 |
| B124 | 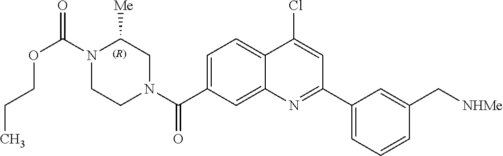 | 0.060 |
| B125 | 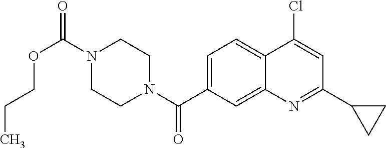 [SMILES: CCCOC(=O)N1CCN(C(=O)c2ccc3c(Cl)cc(C4CC4)nc3c2)CC1] | 0.56 |
| B126 | 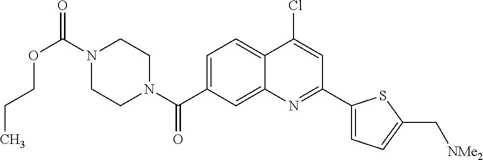 [SMILES: CCCOC(=O)N1CCN(C(=O)c2ccc3c(Cl)cc(-c4ccc(CN(C)C)s4)nc3c2)CC1] | 0.089 |
| B127 | 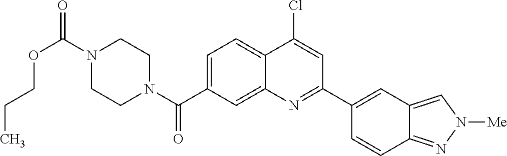 [SMILES: CCCOC(=O)N1CCN(C(=O)c2ccc3c(Cl)cc(-c4ccc5nn(C)cc5c4)nc3c2)CC1] | 0.021 |
| B128 | 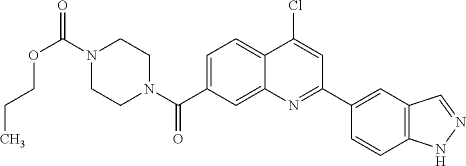 [SMILES: CCCOC(=O)N1CCN(C(=O)c2ccc3c(Cl)cc(-c4ccc5[nH]ncc5c4)nc3c2)CC1] | 0.053 |
| B129 | 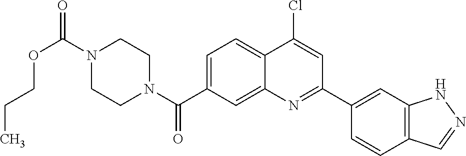 [SMILES: CCCOC(=O)N1CCN(C(=O)c2ccc3c(Cl)cc(-c4ccc5cn[nH]c5c4)nc3c2)CC1] | 0.087 |
| B130 | 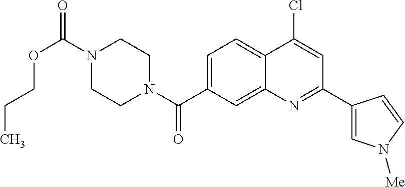 [SMILES: CCCOC(=O)N1CCN(C(=O)c2ccc3c(Cl)cc(-c4ccn(C)c4)nc3c2)CC1] | 0.023 |
| B131 | 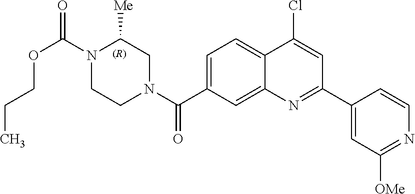 | 0.36 |
| B132 | 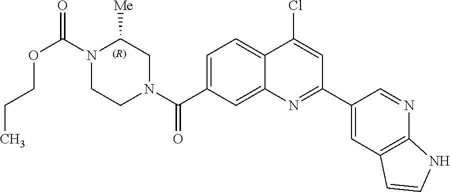 | 0.17 |
| B133 | 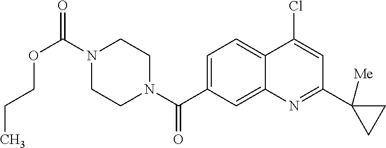 [SMILES: CCCOC(=O)N1CCN(C(=O)c2ccc3c(Cl)cc(C4(C)CC4)nc3c2)CC1] | 1.4 |
| B134 | 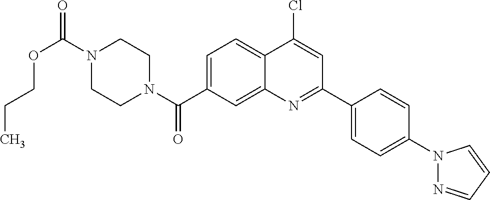 [SMILES: CCCOC(=O)N1CCN(C(=O)c2ccc3c(Cl)cc(-c4ccc(-n5cccn5)cc4)nc3c2)CC1] | 0.6 |
| B135 | 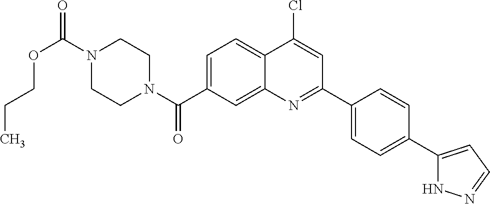 [SMILES: CCCOC(=O)N1CCN(C(=O)c2ccc3c(Cl)cc(-c4ccc(-c5ccn[nH]5)cc4)nc3c2)CC1] | 0.4 |
| B136 | 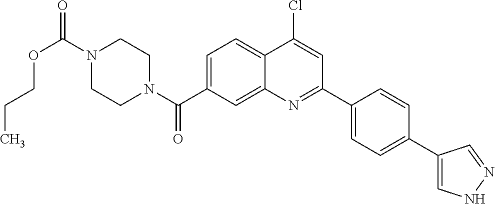 [SMILES: CCCOC(=O)N1CCN(C(=O)c2ccc3c(Cl)cc(-c4ccc(-c5cn[nH]c5)cc4)nc3c2)CC1] | 0.038 |
| B137 | 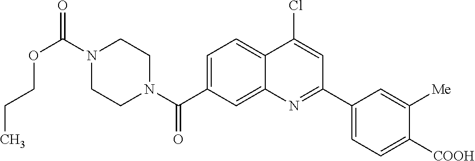 [SMILES: CCCOC(=O)N1CCN(C(=O)c2ccc3c(Cl)cc(-c4ccc(C(=O)O)c(C)c4)nc3c2)CC1] | 3.6 |
| B138 | 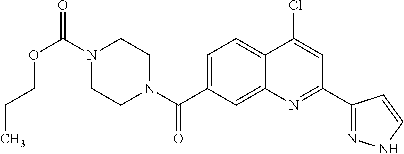 [SMILES: CCCOC(=O)N1CCN(C(=O)c2ccc3c(Cl)cc(-c4cc[nH]n4)nc3c2)CC1] | 0.83 |
| B139 | 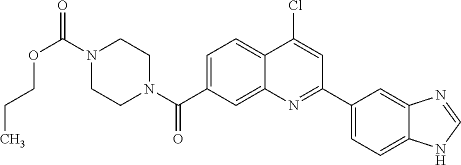 [SMILES: CCCOC(=O)N1CCN(C(=O)c2ccc3c(Cl)cc(-c4ccc5[nH]cnc5c4)nc3c2)CC1] | 0.034 |
| B140 | 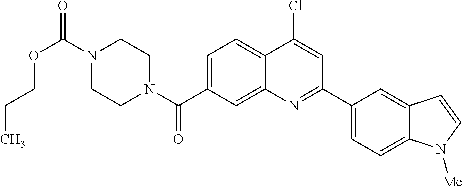 [SMILES: CCCOC(=O)N1CCN(C(=O)c2ccc3c(Cl)cc(-c4ccc5c(ccn5C)c4)nc3c2)CC1] | 0.35 |
| B141 | 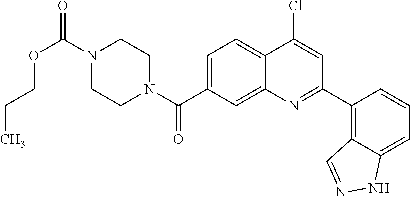 [SMILES: CCCOC(=O)N1CCN(C(=O)c2ccc3c(Cl)cc(-c4cccc5[nH]ncc45)nc3c2)CC1] | 0.63 |
| B142 | 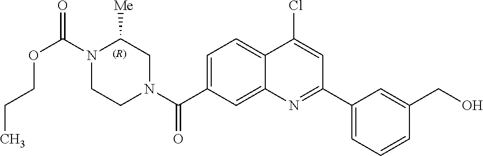 | 0.055 |
| B143 | 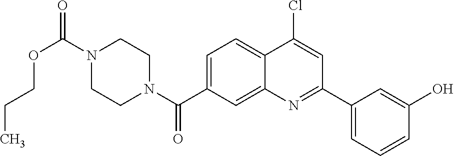 [SMILES: CCCOC(=O)N1CCN(C(=O)c2ccc3c(Cl)cc(-c4cccc(O)c4)nc3c2)CC1] | 0.016 |
| B144 | 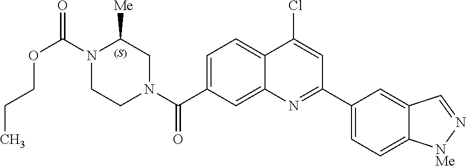 | 0.018 |
| B145 | 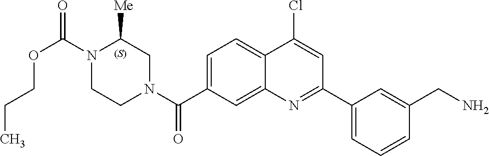 | 0.047 |
| B146 | 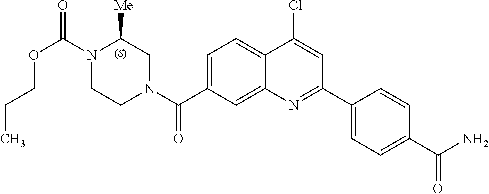 | 0.042 |
| B147 | 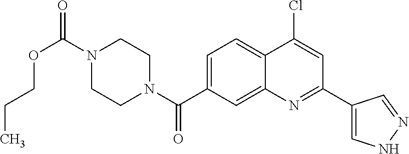 [SMILES: CCCOC(=O)N1CCN(C(=O)c2ccc3c(Cl)cc(-c4cn[nH]c4)nc3c2)CC1] | 0.15 |
| B148 | 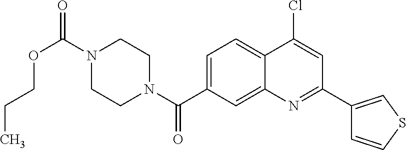 [SMILES: CCCOC(=O)N1CCN(C(=O)c2ccc3c(Cl)cc(-c4ccsc4)nc3c2)CC1] | 0.099 |
| B149 | 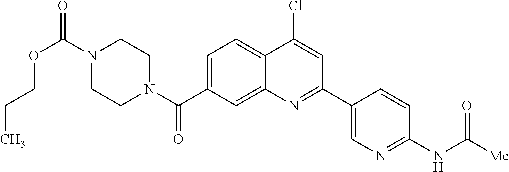 [SMILES: CCCOC(=O)N1CCN(C(=O)c2ccc3c(Cl)cc(-c4ccc(NC(C)=O)nc4)nc3c2)CC1] | 0.11 |
| B150 | 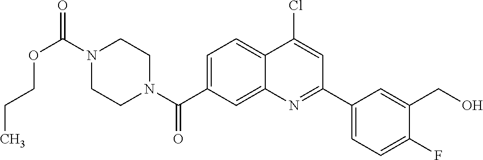 [SMILES: CCCOC(=O)N1CCN(C(=O)c2ccc3c(Cl)cc(-c4ccc(F)c(CO)c4)nc3c2)CC1] | 0.28 |
| B151 | 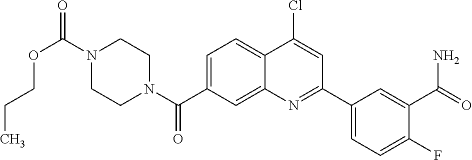 [SMILES: CCCOC(=O)N1CCN(C(=O)c2ccc3c(Cl)cc(-c4ccc(F)c(C(N)=O)c4)nc3c2)CC1] | 0.62 |
| B152 | 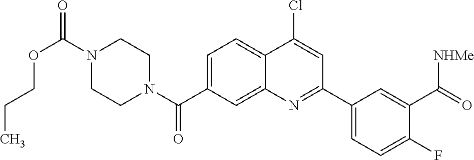 [SMILES: CCCOC(=O)N1CCN(C(=O)c2ccc3c(Cl)cc(-c4ccc(F)c(C(=O)NC)c4)nc3c2)CC1] | 0.41 |
| B153 | 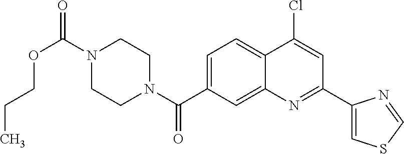 [SMILES: CCCOC(=O)N1CCN(C(=O)c2ccc3c(Cl)cc(-c4cscn4)nc3c2)CC1] | 1.13 |
| B154 | 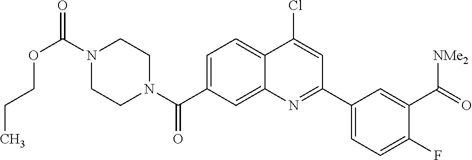 [SMILES: CCCOC(=O)N1CCN(C(=O)c2ccc3c(Cl)cc(-c4ccc(F)c(C(=O)N(C)C)c4)nc3c2)CC1] | 2.29 |
| B155 | 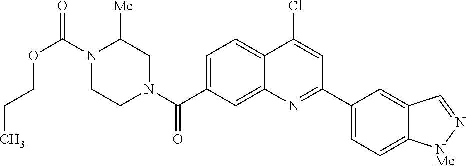 [SMILES: CCCOC(=O)N1CCN(C(=O)c2ccc3c(Cl)cc(-c4ccc5c(cnn5C)c4)nc3c2)CC1C] | 0.18 |
| B156 | 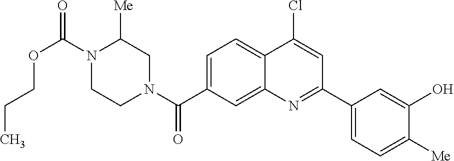 [SMILES: CCCOC(=O)N1CCN(C(=O)c2ccc3c(Cl)cc(-c4ccc(C)c(O)c4)nc3c2)CC1C] | 0.28 |
| B157 | 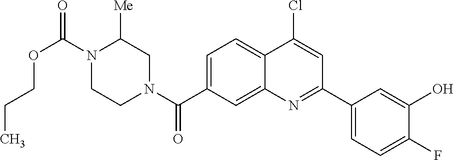 [SMILES: CCCOC(=O)N1CCN(C(=O)c2ccc3c(Cl)cc(-c4ccc(F)c(O)c4)nc3c2)CC1C] | 0.36 |
| B158 | 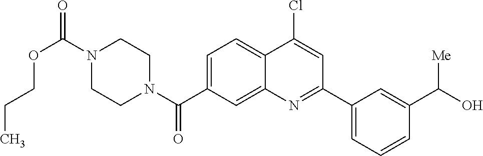 [SMILES: CCCOC(=O)N1CCN(C(=O)c2ccc3c(Cl)cc(-c4cccc(C(C)O)c4)nc3c2)CC1] | 0.084 |
| B159 | 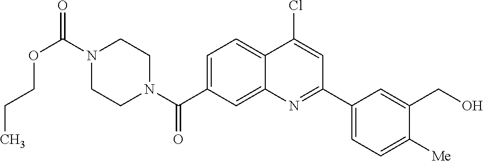 [SMILES: CCCOC(=O)N1CCN(C(=O)c2ccc3c(Cl)cc(-c4ccc(C)c(CO)c4)nc3c2)CC1] | 0.12 |
| B160 | 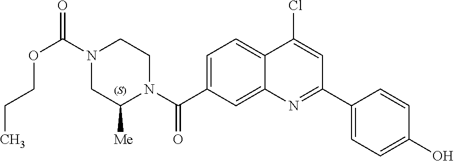 | 0.15 |
| B161 | 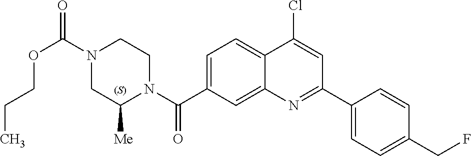 | 0.33 |
| B162 | 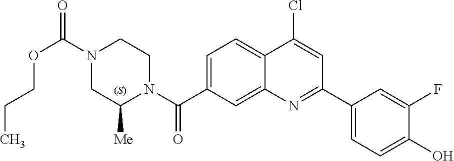 | 0.20 |
| B163 | 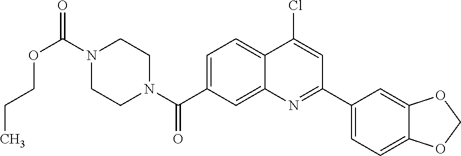 [SMILES: CCCOC(=O)N1CCN(C(=O)c2ccc3c(Cl)cc(-c4ccc5c(c4)OCO5)nc3c2)CC1] | 0.12 |
| B164 | 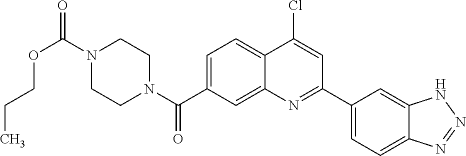 [SMILES: CCCOC(=O)N1CCN(C(=O)c2ccc3c(Cl)cc(-c4ccc5nn[nH]c5c4)nc3c2)CC1] | 0.36 |
| B165 | 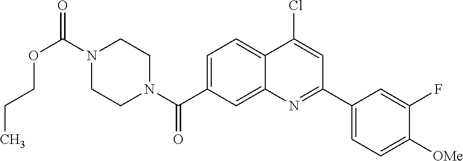 [SMILES: CCCOC(=O)N1CCN(C(=O)c2ccc3c(Cl)cc(-c4ccc(OC)c(F)c4)nc3c2)CC1] | 0.15 |
| B166 | 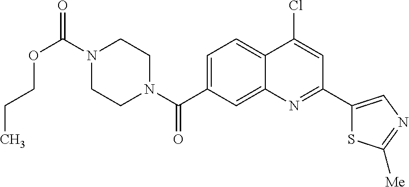 [SMILES: CCCOC(=O)N1CCN(C(=O)c2ccc3c(Cl)cc(-c4cnc(C)s4)nc3c2)CC1] | 3.67 |
| B167 | 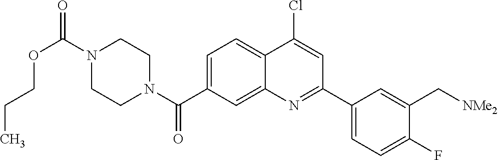 [SMILES: CCCOC(=O)N1CCN(C(=O)c2ccc3c(Cl)cc(-c4ccc(F)c(CN(C)C)c4)nc3c2)CC1] | 0.66 |
| B168 | 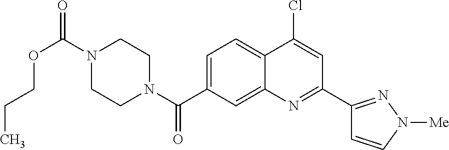 [SMILES: CCCOC(=O)N1CCN(C(=O)c2ccc3c(Cl)cc(-c4ccn(C)n4)nc3c2)CC1] | 1.75 |
| B169 | 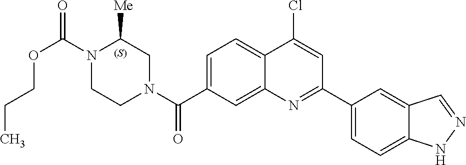 | 0.15 |
| B170 | 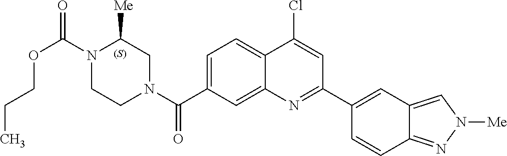 | 0.26 |
| B171 | 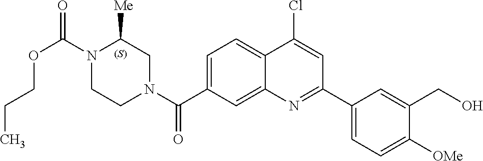 | 0.15 |
| B172 | 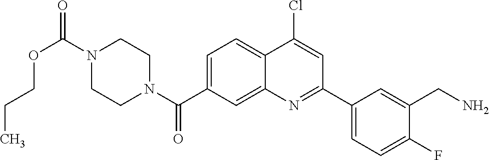 [SMILES: CCCOC(=O)N1CCN(C(=O)c2ccc3c(Cl)cc(-c4ccc(F)c(CN)c4)nc3c2)CC1] | 0.039 |
| B173 | 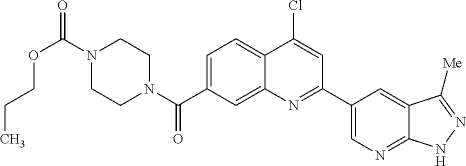 [SMILES: CCCOC(=O)N1CCN(C(=O)c2ccc3c(Cl)cc(-c4cnc5[nH]nc(C)c5c4)nc3c2)CC1] | 2.5 |
| B174 | 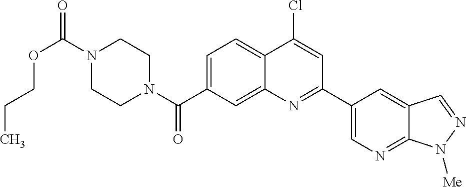 [SMILES: CCCOC(=O)N1CCN(C(=O)c2ccc3c(Cl)cc(-c4cnc5c(cnn5C)c4)nc3c2)CC1] | 0.47 |
| B175 | 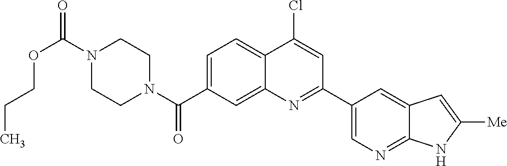 [SMILES: CCCOC(=O)N1CCN(C(=O)c2ccc3c(Cl)cc(-c4cnc5[nH]c(C)cc5c4)nc3c2)CC1] | 0.40 |
| B176 | 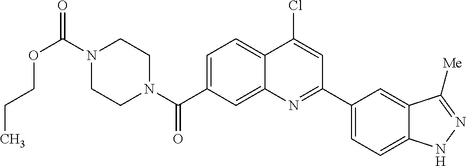 [SMILES: CCCOC(=O)N1CCN(C(=O)c2ccc3c(Cl)cc(-c4ccc5[nH]nc(C)c5c4)nc3c2)CC1] | 0.49 |
| B177 | 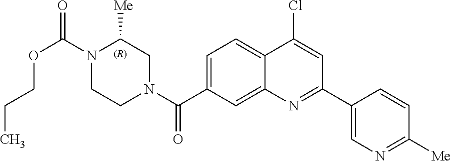 | 0.36 |
| B178 | 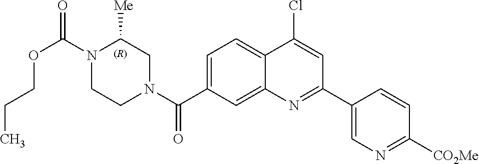 | 0.93 |
| B179 | 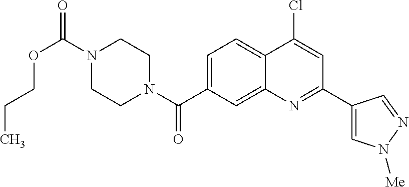 [SMILES: CCCOC(=O)N1CCN(C(=O)c2ccc3c(Cl)cc(-c4cnn(C)c4)nc3c2)CC1] | 0.25 |
| B180 | 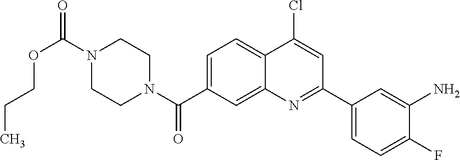 [SMILES: CCCOC(=O)N1CCN(C(=O)c2ccc3c(Cl)cc(-c4ccc(F)c(N)c4)nc3c2)CC1] | 0.25 |
| B181 | 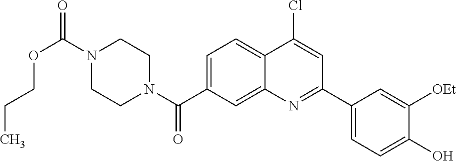 [SMILES: CCCOC(=O)N1CCN(C(=O)c2ccc3c(Cl)cc(-c4ccc(O)c(OCC)c4)nc3c2)CC1] | 0.29 |
| B182 | 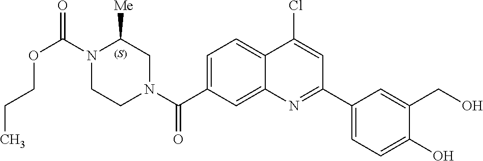 | 0.14 |
| B183 | 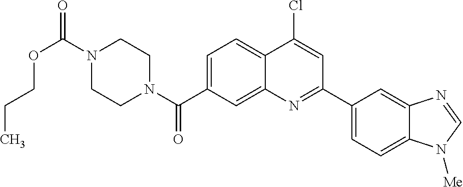 [SMILES: CCCOC(=O)N1CCN(C(=O)c2ccc3c(Cl)cc(-c4ccc5c(c4)ncn5C)nc3c2)CC1] | 0.12 |
| B184 | 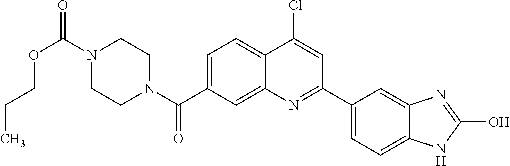 [SMILES: CCCOC(=O)N1CCN(C(=O)c2ccc3c(Cl)cc(-c4ccc5[nH]c(O)nc5c4)nc3c2)CC1] | 0.10 |
| B185 | 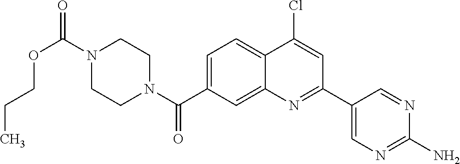 [SMILES: CCCOC(=O)N1CCN(C(=O)c2ccc3c(Cl)cc(-c4cnc(N)nc4)nc3c2)CC1] | 0.87 |
| B186 | 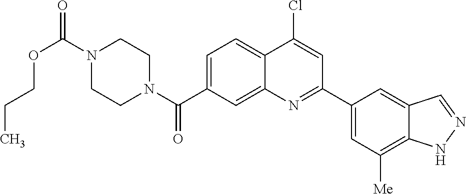 [SMILES: CCCOC(=O)N1CCN(C(=O)c2ccc3c(Cl)cc(-c4cc(C)c5[nH]ncc5c4)nc3c2)CC1] | 0.14 |
| B187 | 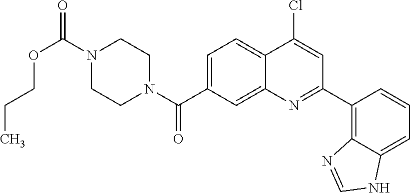 [SMILES: CCCOC(=O)N1CCN(C(=O)c2ccc3c(Cl)cc(-c4cccc5[nH]cnc45)nc3c2)CC1] | 0.14 |
| B188 | 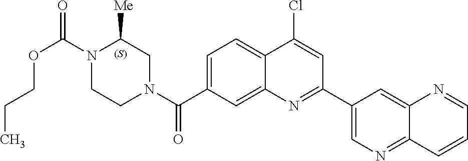 | 0.89 |
| B189 | 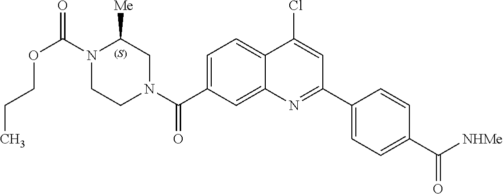 | 0.069 |
| B190 | 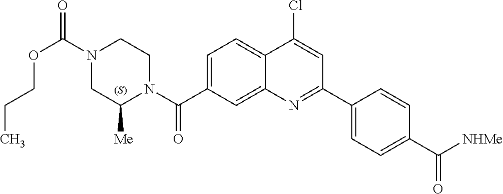 | 0.13 |
| B191 | 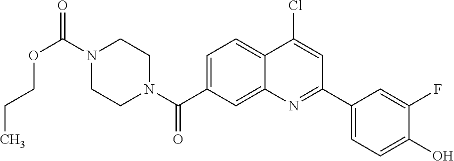 [SMILES: CCCOC(=O)N1CCN(C(=O)c2ccc3c(Cl)cc(-c4ccc(O)c(F)c4)nc3c2)CC1] | 0.14 |
| B192 | 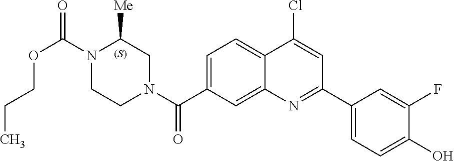 | 0.075 |
| B193 | 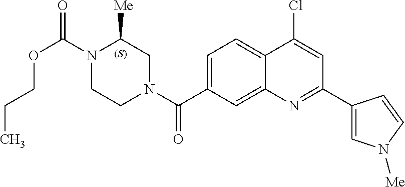 | 0.060 |
| B194 | 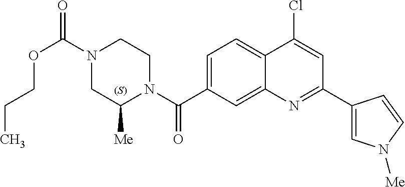 | 0.13 |
| B195 | 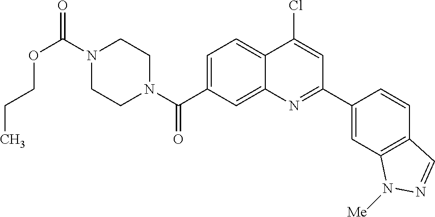 [SMILES: CCCOC(=O)N1CCN(C(=O)c2ccc3c(Cl)cc(-c4ccc5cnn(C)c5c4)nc3c2)CC1] | 0.25 |
| B196 | 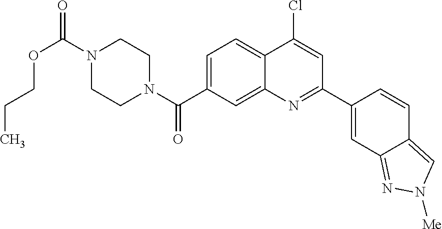 [SMILES: CCCOC(=O)N1CCN(C(=O)c2ccc3c(Cl)cc(-c4ccc5cn(C)nc5c4)nc3c2)CC1] | 0.056 |
| B197 | 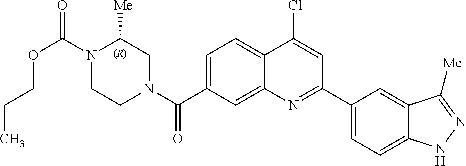 | 0.10 |
| B198 | 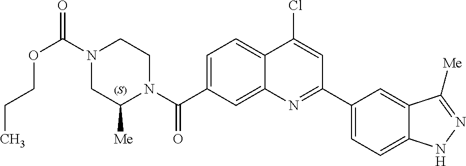 | 0.20 |
| B199 | 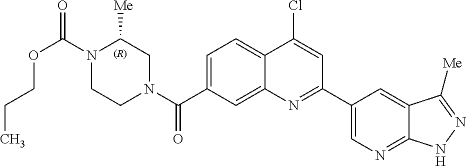 | 0.35 |
| B200 | 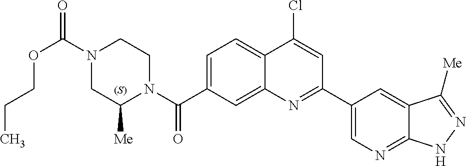 | 1.03 |
| B201 | 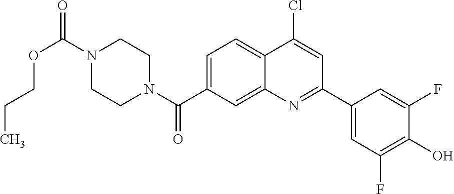 [SMILES: CCCOC(=O)N1CCN(C(=O)c2ccc3c(Cl)cc(-c4cc(F)c(O)c(F)c4)nc3c2)CC1] | 0.18 |
| B202 | 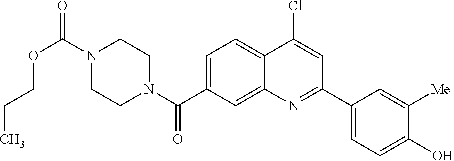 [SMILES: CCCOC(=O)N1CCN(C(=O)c2ccc3c(Cl)cc(-c4ccc(O)c(C)c4)nc3c2)CC1] | 0.12 |
| B203 | 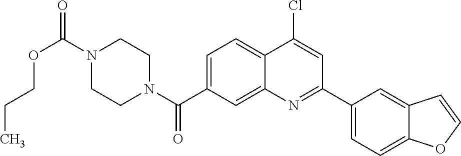 [SMILES: CCCOC(=O)N1CCN(C(=O)c2ccc3c(Cl)cc(-c4ccc5occc5c4)nc3c2)CC1] | 0.46 |
| B204 | 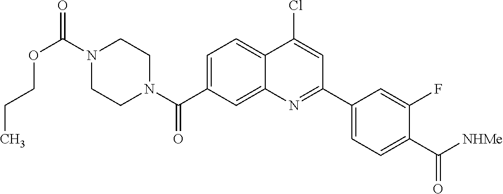 [SMILES: CCCOC(=O)N1CCN(C(=O)c2ccc3c(Cl)cc(-c4ccc(C(=O)NC)c(F)c4)nc3c2)CC1] | 1.01 |
| B205 | 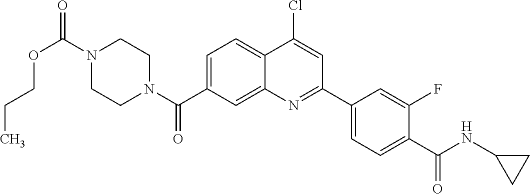 [SMILES: CCCOC(=O)N1CCN(C(=O)c2ccc3c(Cl)cc(-c4ccc(C(=O)NC5CC5)c(F)c4)nc3c2)CC1] | 0.89 |
| B206 | 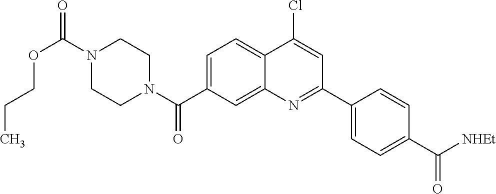 | 0.60 |
| B207 | 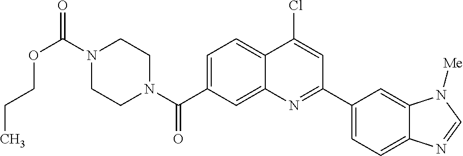 [SMILES: CCCOC(=O)N1CCN(C(=O)c2ccc3c(Cl)cc(-c4ccc5ncn(C)c5c4)nc3c2)CC1] | 0.060 |
| B208 | 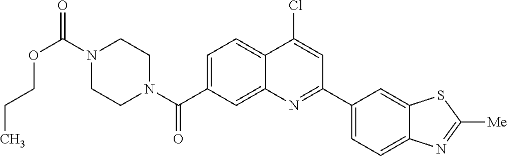 [SMILES: CCCOC(=O)N1CCN(C(=O)c2ccc3c(Cl)cc(-c4ccc5nc(C)sc5c4)nc3c2)CC1] | 0.38 |
| B209 | 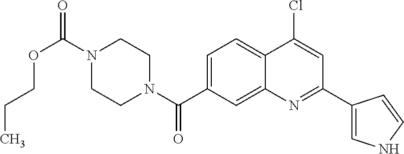 [SMILES: CCCOC(=O)N1CCN(C(=O)c2ccc3c(Cl)cc(-c4cc[nH]c4)nc3c2)CC1] | 0.19 |
| B210 | 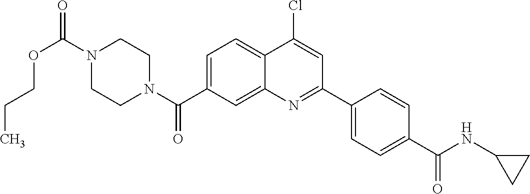 [SMILES: CCCOC(=O)N1CCN(C(=O)c2ccc3c(Cl)cc(-c4ccc(C(=O)NC5CC5)cc4)nc3c2)CC1] | 0.026 |
| B211 | 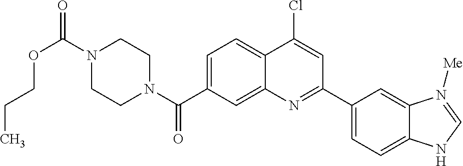 | 1.01 |
| B212 | 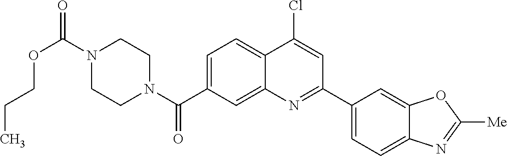 [SMILES: CCCOC(=O)N1CCN(C(=O)c2ccc3c(Cl)cc(-c4ccc5nc(C)oc5c4)nc3c2)CC1] | 0.24 |
| B213 | 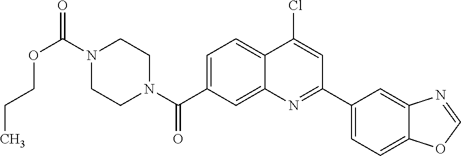 [SMILES: CCCOC(=O)N1CCN(C(=O)c2ccc3c(Cl)cc(-c4ccc5ocnc5c4)nc3c2)CC1] | 0.33 |
| B214 | 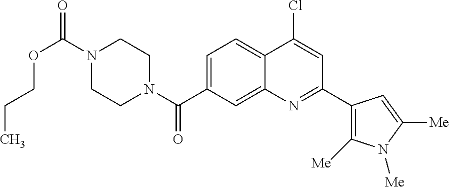 [SMILES: CCCOC(=O)N1CCN(C(=O)c2ccc3c(Cl)cc(-c4cc(C)n(C)c4C)nc3c2)CC1] | 0.12 |
| B215 | 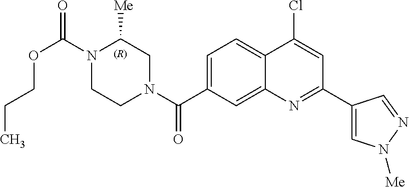 | 0.11 |
| B216 | 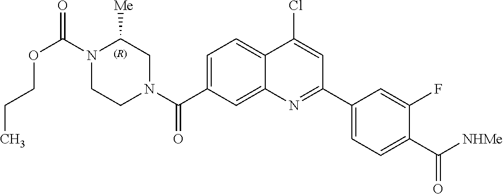 | 0.23 |
| B217 | 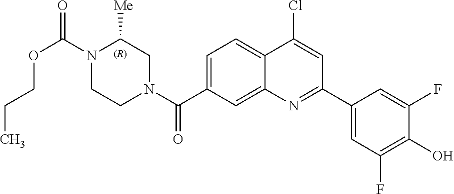 | 0.13 |
| B218 | 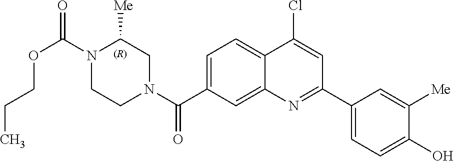 | 0.22 |
| B219 | 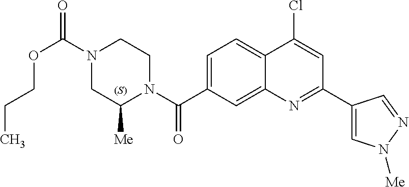 | 0.23 |
| B220 | 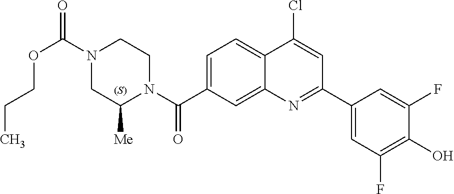 | 0.35 |
| B221 | 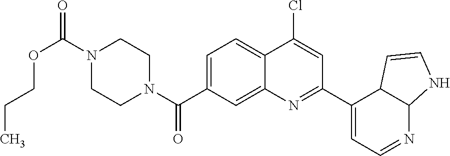 [SMILES: CCCOC(=O)N1CCN(C(=O)c2ccc3c(Cl)cc(C4=CC=NC5NC=CC45)nc3c2)CC1] | 0.80 |
| B222 | 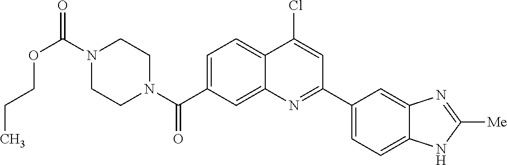 [SMILES: CCCOC(=O)N1CCN(C(=O)c2ccc3c(Cl)cc(-c4ccc5[nH]c(C)nc5c4)nc3c2)CC1] | 0.046 |
| B223 | 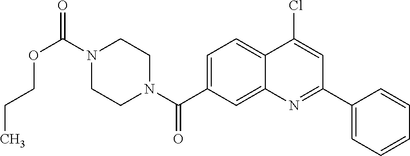 [SMILES: CCCOC(=O)N1CCN(C(=O)c2ccc3c(Cl)cc(-c4ccccc4)nc3c2)CC1] | 0.29 |
| B224 | 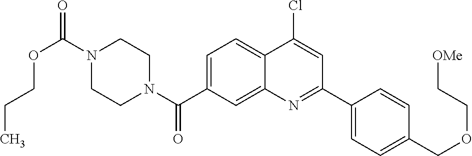 [SMILES: CCCOC(=O)N1CCN(C(=O)c2ccc3c(Cl)cc(-c4ccc(COCCOC)cc4)nc3c2)CC1] | 0.80 |
| B225 | 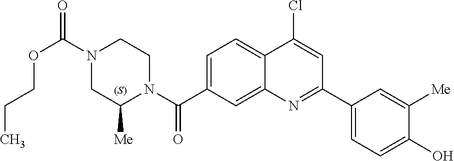 | 0.23 |
| B226 | 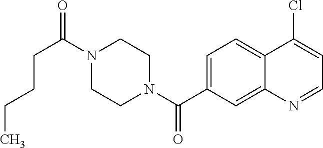 [SMILES: CCCCC(=O)N1CCN(C(=O)c2ccc3c(Cl)ccnc3c2)CC1] | 7.9 |
| B227 | 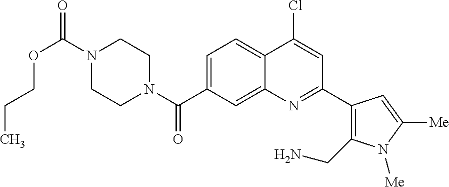 [SMILES: CCCOC(=O)N1CCN(C(=O)c2ccc3c(Cl)cc(-c4cc(C)n(C)c4CN)nc3c2)CC1] | 0.77 |
| B228 | 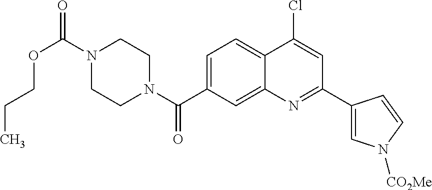 [SMILES: CCCOC(=O)N1CCN(C(=O)c2ccc3c(Cl)cc(-c4ccn(C(=O)OC)c4)nc3c2)CC1] | 0.30 |
| B229 | 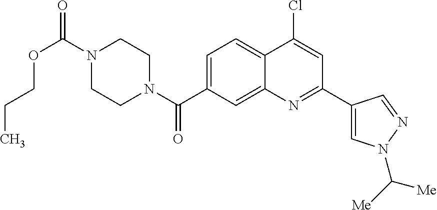 [SMILES: CCCOC(=O)N1CCN(C(=O)c2ccc3c(Cl)cc(-c4cnn(C(C)C)c4)nc3c2)CC1] | 1.1 |
| B230 | 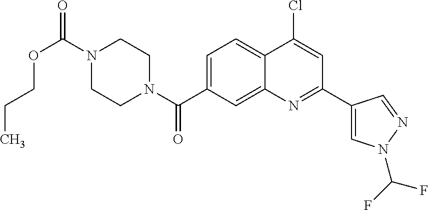 [SMILES: CCCOC(=O)N1CCN(C(=O)c2ccc3c(Cl)cc(-c4cnn(C(F)F)c4)nc3c2)CC1] | 0.70 |
| B231 | 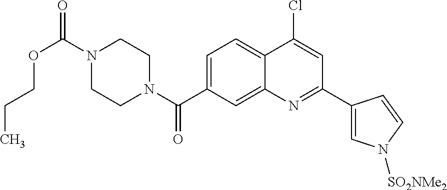 | 0.63 |
| B232 | 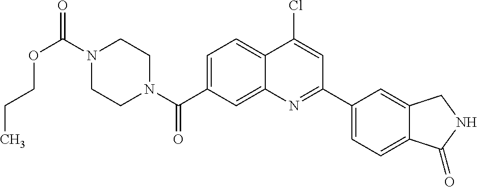 [SMILES: CCCOC(=O)N1CCN(C(=O)c2ccc3c(Cl)cc(-c4ccc5c(c4)CNC5=O)nc3c2)CC1] | 0.15 |
| B233 | 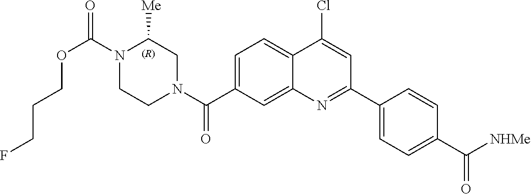 | 0.16 |
| B234 | 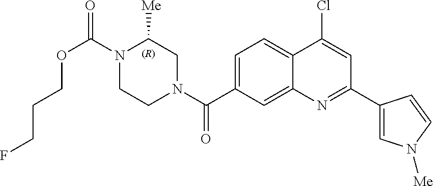 | 0.098 |
| B235 | 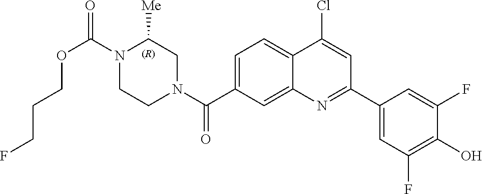 | 0.23 |
| B236 | 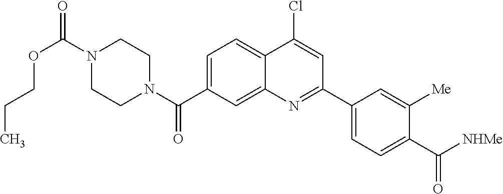 [SMILES: CCCOC(=O)N1CCN(C(=O)c2ccc3c(Cl)cc(-c4ccc(C(=O)NC)c(C)c4)nc3c2)CC1] | 0.36 |
| B237 | 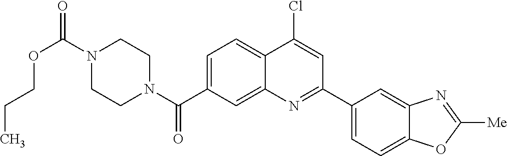 [SMILES: CCCOC(=O)N1CCN(C(=O)c2ccc3c(Cl)cc(-c4ccc5oc(C)nc5c4)nc3c2)CC1] | 0.20 |
| B238 | 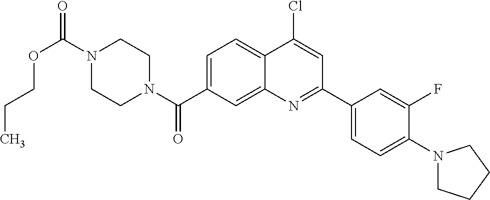 [SMILES: CCCOC(=O)N1CCN(C(=O)c2ccc3c(Cl)cc(-c4ccc(N5CCCC5)c(F)c4)nc3c2)CC1] | 3.5 |
| B239 | 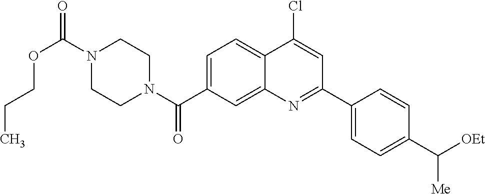 [SMILES: CCCOC(=O)N1CCN(C(=O)c2ccc3c(Cl)cc(-c4ccc(C(C)OCC)cc4)nc3c2)CC1] | 1.3 |
| B240 | 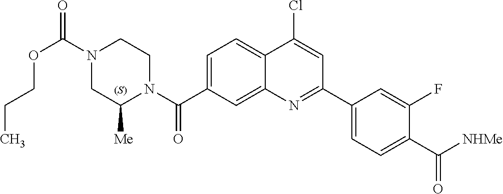 | 0.25 |
| B241 | 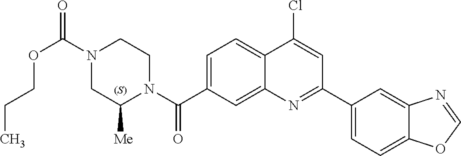 | 0.24 |
| B242 | 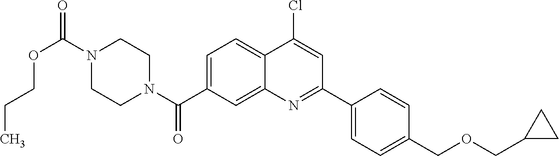 [SMILES: CCCOC(=O)N1CCN(C(=O)c2ccc3c(Cl)cc(-c4ccc(COCC5CC5)cc4)nc3c2)CC1] | 0.67 |
| B243 | 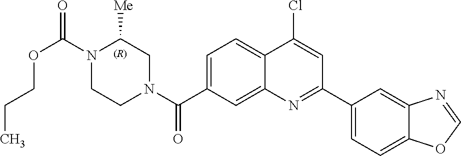 | 0.063 |
| B244 | 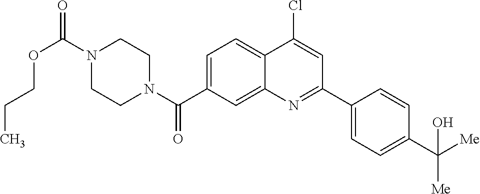 [SMILES: CCCOC(=O)N1CCN(C(=O)c2ccc3c(Cl)cc(-c4ccc(C(C)(C)O)cc4)nc3c2)CC1] | 0.14 |
| B245 | 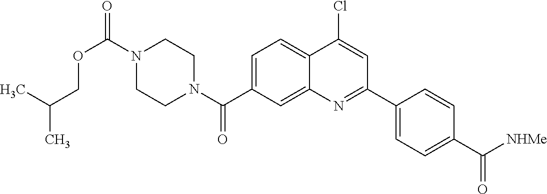 [SMILES: CNC(=O)c1ccc(-c2cc(Cl)c3ccc(C(=O)N4CCN(C(=O)OCC(C)C)CC4)cc3n2)cc1] | 1.01 |
| B246 | 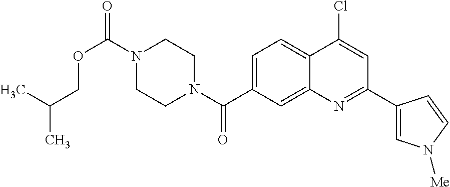 [SMILES: CC(C)COC(=O)N1CCN(C(=O)c2ccc3c(Cl)cc(-c4ccn(C)c4)nc3c2)CC1] | 0.27 |
| B247 | 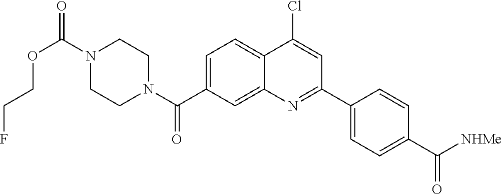 [SMILES: CNC(=O)c1ccc(-c2cc(Cl)c3ccc(C(=O)N4CCN(C(=O)OCCF)CC4)cc3n2)cc1] | 7.4 |
| B248 | 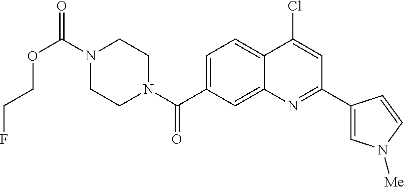 [SMILES: Cn1ccc(-c2cc(Cl)c3ccc(C(=O)N4CCN(C(=O)OCCF)CC4)cc3n2)c1] | 0.14 |
| B249 | 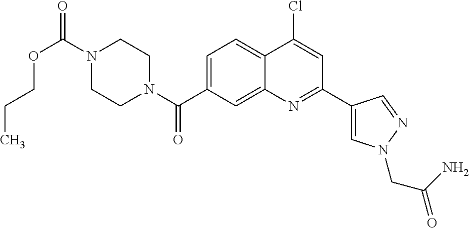 [SMILES: CCCOC(=O)N1CCN(C(=O)c2ccc3c(Cl)cc(-c4cnn(CC(N)=O)c4)nc3c2)CC1] | 0.19 |
| B250 | 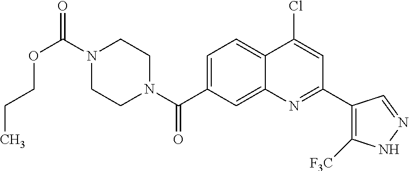 [SMILES: CCCOC(=O)N1CCN(C(=O)c2ccc3c(Cl)cc(-c4cn[nH]c4C(F)(F)F)nc3c2)CC1] | 4.9 |
| B251 | 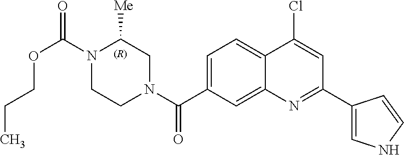 | 0.043 |
| B252 | 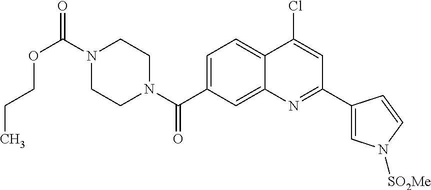 [SMILES: CCCOC(=O)N1CCN(C(=O)c2ccc3c(Cl)cc(-c4ccn(S(C)(=O)=O)c4)nc3c2)CC1] | 0.52 |
| B253 | 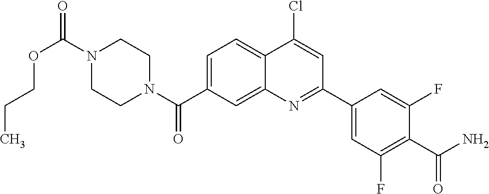 [SMILES: CCCOC(=O)N1CCN(C(=O)c2ccc3c(Cl)cc(-c4cc(F)c(C(N)=O)c(F)c4)nc3c2)CC1] | 0.49 |
| B254 | 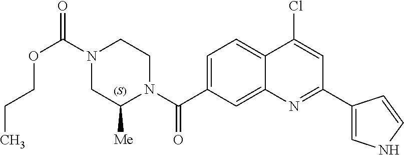 | 0.054 |
| B255 | 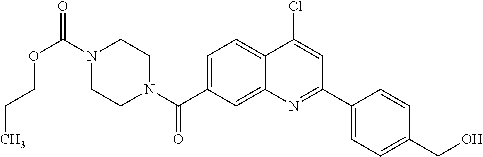 [SMILES: CCCOC(=O)N1CCN(C(=O)c2ccc3c(Cl)cc(-c4ccc(CO)cc4)nc3c2)CC1] | 0.028 |
| B256 | 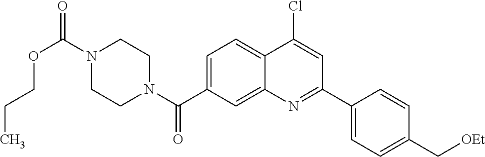 [SMILES: CCCOC(=O)N1CCN(C(=O)c2ccc3c(Cl)cc(-c4ccc(COCC)cc4)nc3c2)CC1] | 0.50 |
| B257 | 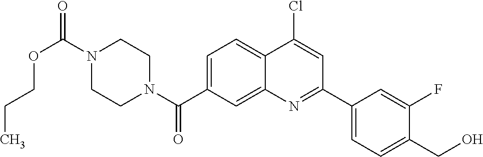 [SMILES: CCCOC(=O)N1CCN(C(=O)c2ccc3c(Cl)cc(-c4ccc(CO)c(F)c4)nc3c2)CC1] | 0.15 |
| B258 | 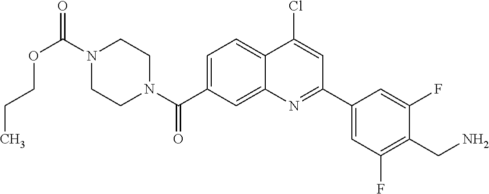 [SMILES: CCCOC(=O)N1CCN(C(=O)c2ccc3c(Cl)cc(-c4cc(F)c(CN)c(F)c4)nc3c2)CC1] | 0.85 |
| B259 | 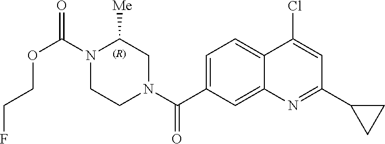 | 2.6 |
| B260 | 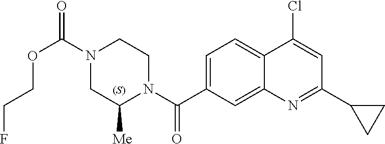 | 4.9 |
| B261 | 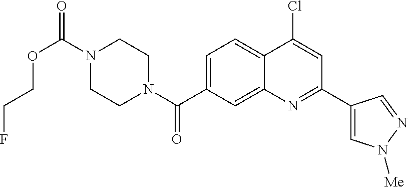 [SMILES: Cn1cc(-c2cc(Cl)c3ccc(C(=O)N4CCN(C(=O)OCCF)CC4)cc3n2)cn1] | 2.7 |
| B262 | 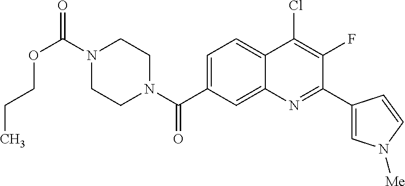 [SMILES: CCCOC(=O)N1CCN(C(=O)c2ccc3c(Cl)c(F)c(-c4ccn(C)c4)nc3c2)CC1] | 0.39 |
| B263 | 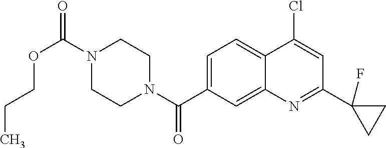 [SMILES: CCCOC(=O)N1CCN(C(=O)c2ccc3c(Cl)cc(C4(F)CC4)nc3c2)CC1] | 13.9 |
| B264 | 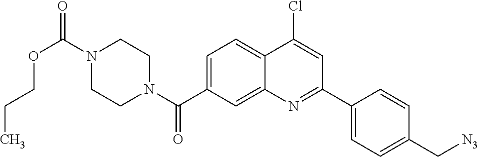 [SMILES: CCCOC(=O)N1CCN(C(=O)c2ccc3c(Cl)cc(-c4ccc(CN=[N+]=[N-])cc4)nc3c2)CC1] | 0.084 |
| B265 | 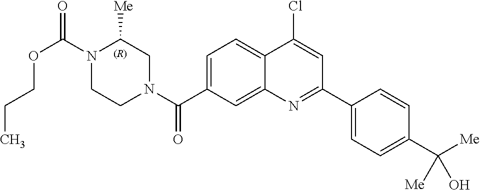 | 0.067 |
| B266 | 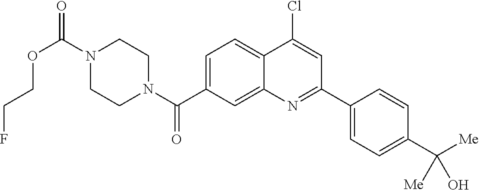 [SMILES: CC(C)(O)c1ccc(-c2cc(Cl)c3ccc(C(=O)N4CCN(C(=O)OCCF)CC4)cc3n2)cc1] | 3.3 |
| B267 | 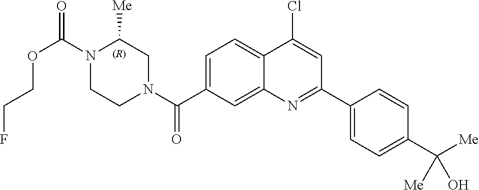 | 0.15 |
| B268 | 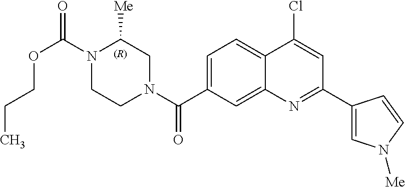 | 0.017 |
| B269 | 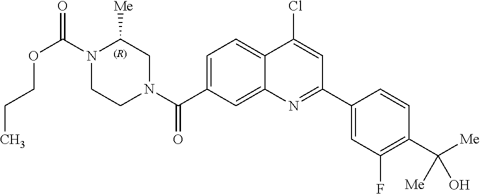 | 0.17 |
| B270 | 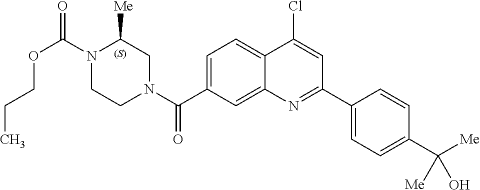 | 0.016 |
| B271 | 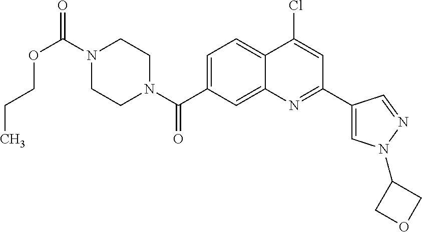 [SMILES: CCCOC(=O)N1CCN(C(=O)c2ccc3c(Cl)cc(-c4cnn(C5COC5)c4)nc3c2)CC1] | 0.034 |
| B272 | 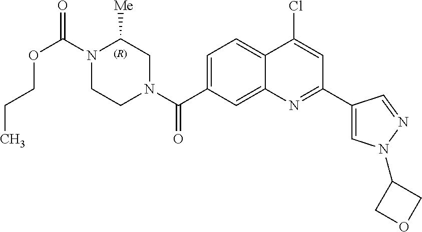 | 0.016 |
| B273 | 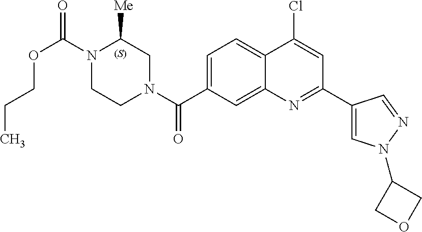 | 0.019 |
| B274 | 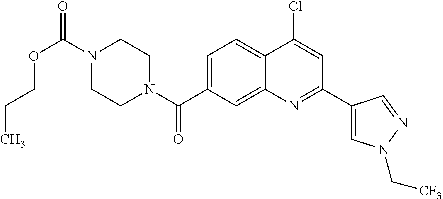 [SMILES: CCCOC(=O)N1CCN(C(=O)c2ccc3c(Cl)cc(-c4cnn(CC(F)(F)F)c4)nc3c2)CC1] | 0.21 |
| B275 | 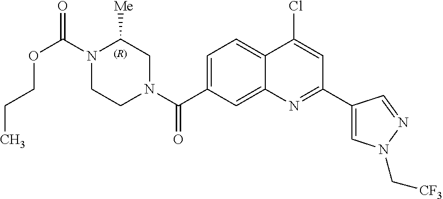 | 0.034 |
| B276 | 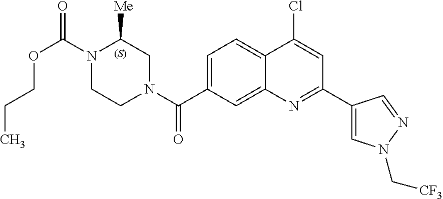 | 0.022 |
| B277 | 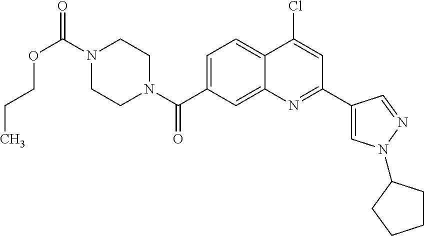 [SMILES: CCCOC(=O)N1CCN(C(=O)c2ccc3c(Cl)cc(-c4cnn(C5CCCC5)c4)nc3c2)CC1] | 0.060 |
| B278 | 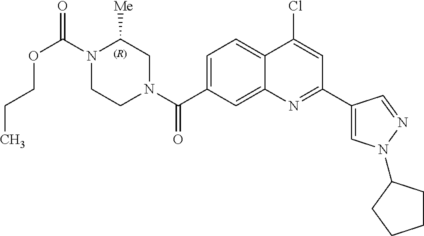 | 0.022 |
| B279 | 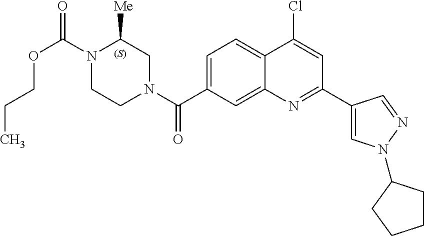 | 0.020 |
| B280 | 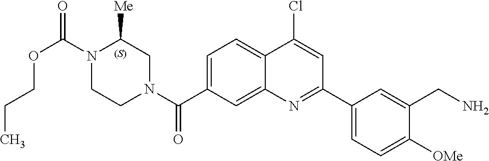 | 0.067 |
| B281 | 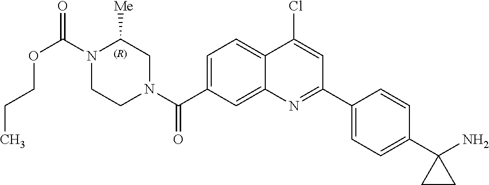 | 0.012 |
| B282 | 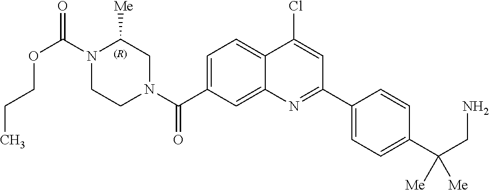 | 0.013 |
| B283 | 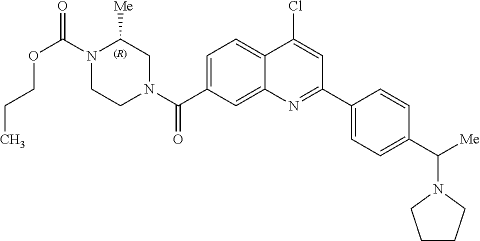 | 0.28 |
| B284 | 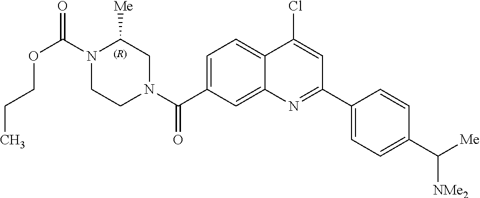 | 0.024 |
| B285 | 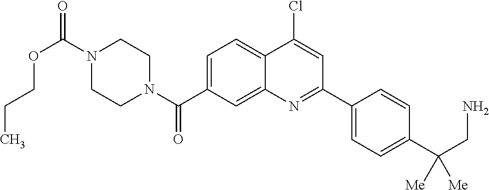 [SMILES: CCCOC(=O)N1CCN(C(=O)c2ccc3c(Cl)cc(-c4ccc(C(C)(C)CN)cc4)nc3c2)CC1] | 0.013 |
| D001 | 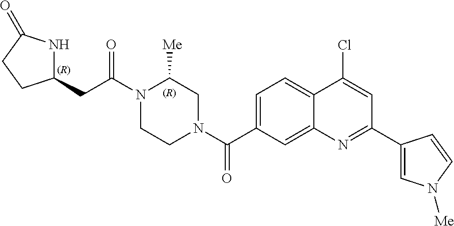 | 13.6 |
| D002 | 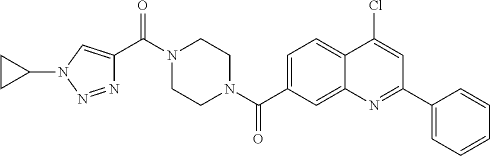 [SMILES: O=C(c1ccc2c(Cl)cc(-c3ccccc3)nc2c1)N1CCN(C(=O)c2cn(C3CC3)nn2)CC1] | 0.004 |
| D003 | 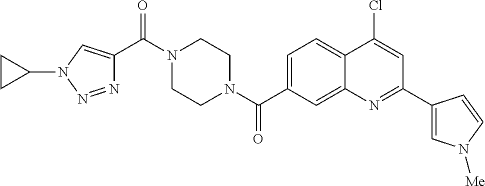 [SMILES: Cn1ccc(-c2cc(Cl)c3ccc(C(=O)N4CCN(C(=O)c5cn(C6CC6)nn5)CC4)cc3n2)c1] | 0.025 |
| D004 | 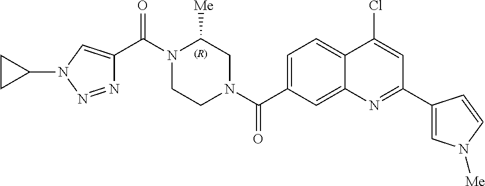 | 0.013 |
| D005 | 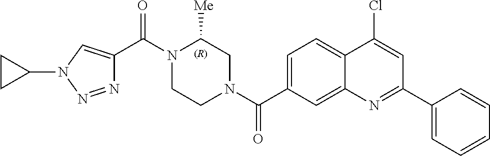 | 0.077 |
| D006 | 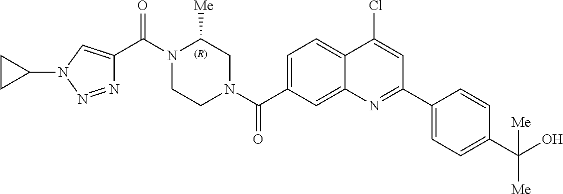 | 0.052 |
| D007 | 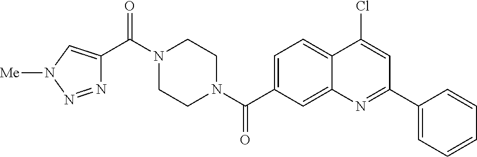 [SMILES: Cn1cc(C(=O)N2CCN(C(=O)c3ccc4c(Cl)cc(-c5ccccc5)nc4c3)CC2)nn1] | 0.083 |
| D008 | 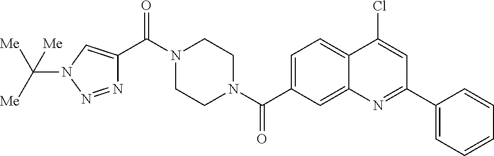 [SMILES: CC(C)(C)n1cc(C(=O)N2CCN(C(=O)c3ccc4c(Cl)cc(-c5ccccc5)nc4c3)CC2)nn1] | 0.45 |
| D009 | 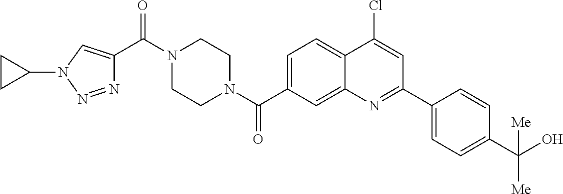 [SMILES: CC(C)(O)c1ccc(-c2cc(Cl)c3ccc(C(=O)N4CCN(C(=O)c5cn(C6CC6)nn5)CC4)cc3n2)cc1] | 0.27 |
| D010 | 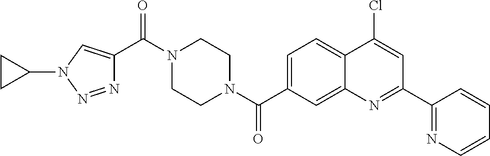 [SMILES: O=C(c1ccc2c(Cl)cc(-c3ccccn3)nc2c1)N1CCN(C(=O)c2cn(C3CC3)nn2)CC1] | 0.082 |
| D011 | 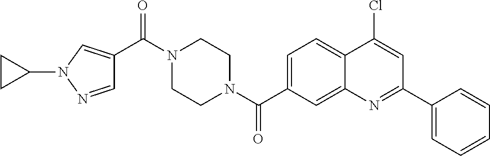 [SMILES: O=C(c1ccc2c(Cl)cc(-c3ccccc3)nc2c1)N1CCN(C(=O)c2cnn(C3CC3)c2)CC1] | 0.20 |
| B286 | 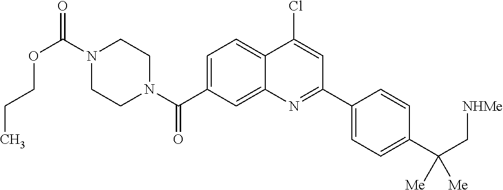 [SMILES: CCCOC(=O)N1CCN(C(=O)c2ccc3c(Cl)cc(-c4ccc(C(C)(C)CNC)cc4)nc3c2)CC1] | 0.025 |
| B287 | 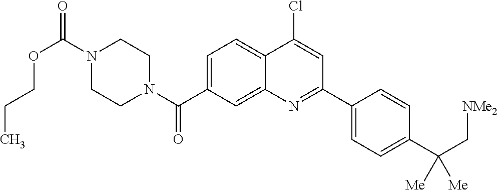 [SMILES: CCCOC(=O)N1CCN(C(=O)c2ccc3c(Cl)cc(-c4ccc(C(C)(C)CN(C)C)cc4)nc3c2)CC1] | 0.13 |
| D012 | 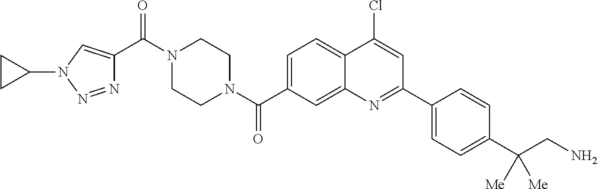 [SMILES: CC(C)(CN)c1ccc(-c2cc(Cl)c3ccc(C(=O)N4CCN(C(=O)c5cn(C6CC6)nn5)CC4)cc3n2)cc1] | 0.10 |
| D013 | 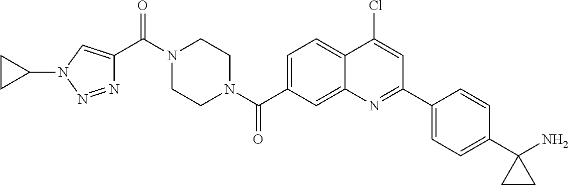 [SMILES: NC1(c2ccc(-c3cc(Cl)c4ccc(C(=O)N5CCN(C(=O)c6cn(C7CC7)nn6)CC5)cc4n3)cc2)CC1] | 0.078 |
| D014 | 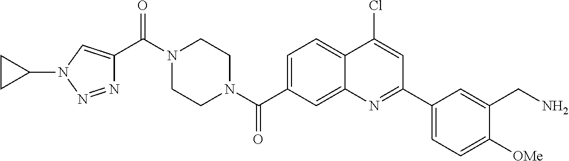 [SMILES: COc1ccc(-c2cc(Cl)c3ccc(C(=O)N4CCN(C(=O)c5cn(C6CC6)nn5)CC4)cc3n2)cc1CN] | 0.069 |
| E001 | 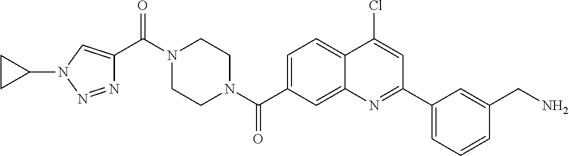 [SMILES: NCc1cccc(-c2cc(Cl)c3ccc(C(=O)N4CCN(C(=O)c5cn(C6CC6)nn5)CC4)cc3n2)c1] | |
| E002 | 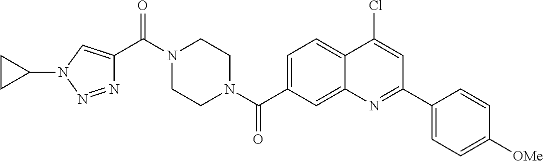 [SMILES: COc1ccc(-c2cc(Cl)c3ccc(C(=O)N4CCN(C(=O)c5cn(C6CC6)nn5)CC4)cc3n2)cc1] | |
| E003 | 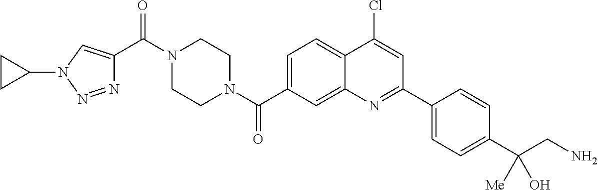 [SMILES: CC(O)(CN)c1ccc(-c2cc(Cl)c3ccc(C(=O)N4CCN(C(=O)c5cn(C6CC6)nn5)CC4)cc3n2)cc1] | |
| E004 | 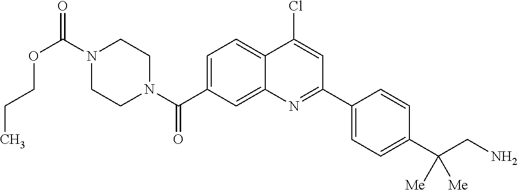 [SMILES: CCCOC(=O)N1CCN(C(=O)c2ccc3c(Cl)cc(-c4ccc(C(C)(C)CN)cc4)nc3c2)CC1] | |
| E005 | 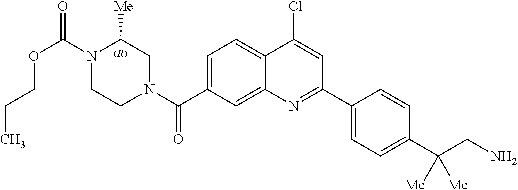 | |
| E006 | 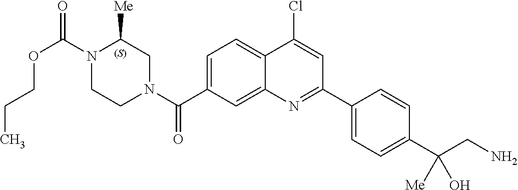 | |
| E007 | 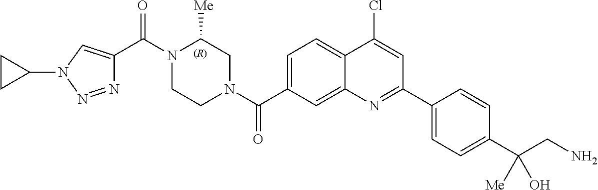 | |
| E008 | 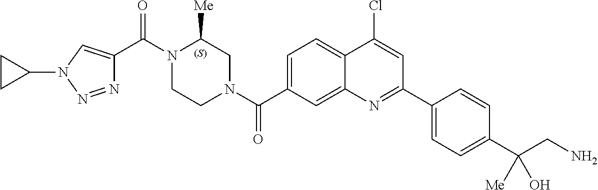 | |
| E009 | 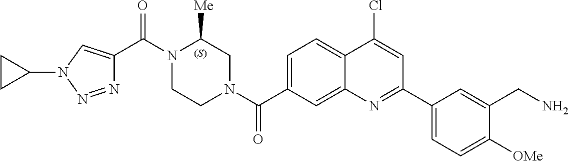 | |
| E010 | 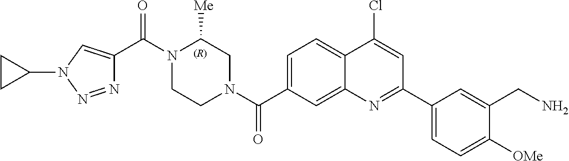 | |
| E011 | 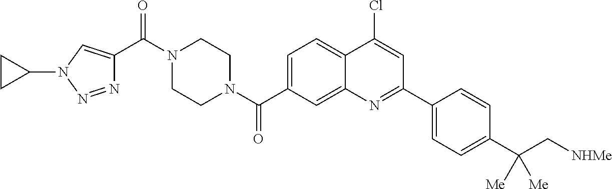 [SMILES: CNCC(C)(C)c1ccc(-c2cc(Cl)c3ccc(C(=O)N4CCN(C(=O)c5cn(C6CC6)nn5)CC4)cc3n2)cc1] | |
| E012 | 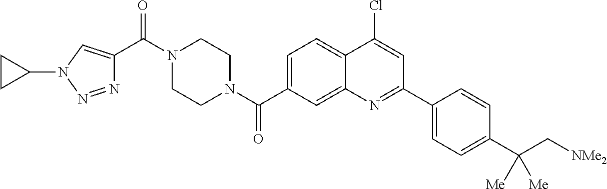 [SMILES: CN(C)CC(C)(C)c1ccc(-c2cc(Cl)c3ccc(C(=O)N4CCN(C(=O)c5cn(C6CC6)nn5)CC4)cc3n2)cc1] | |
| E013 | 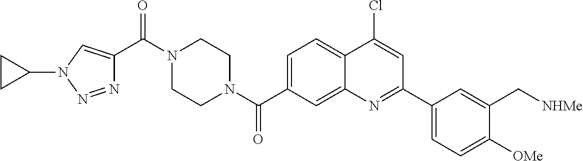 [SMILES: CNCc1cc(-c2cc(Cl)c3ccc(C(=O)N4CCN(C(=O)c5cn(C6CC6)nn5)CC4)cc3n2)ccc1OC] | |
| E014 | 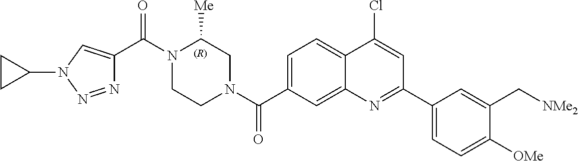 | |
| E015 | 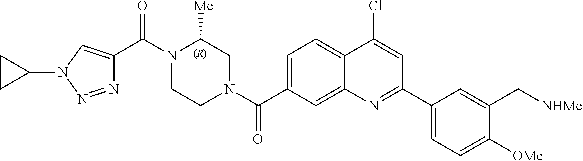 | |
| E015 | 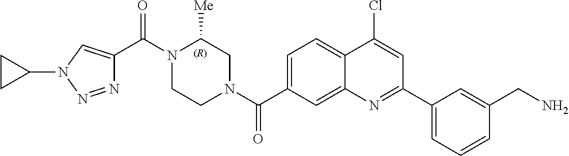 | |
| E016 | 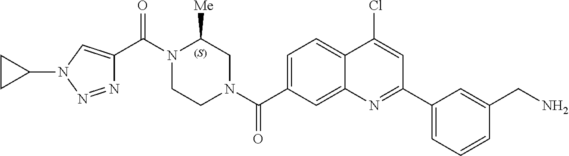 | |
| E017 | 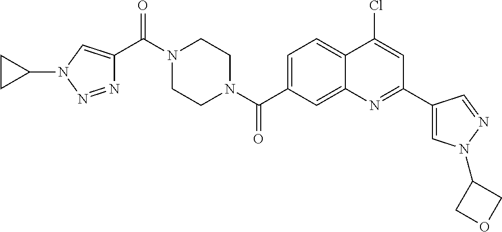 [SMILES: O=C(c1ccc2c(Cl)cc(-c3cnn(C4COC4)c3)nc2c1)N1CCN(C(=O)c2cn(C3CC3)nn2)CC1] | |
| E018 | 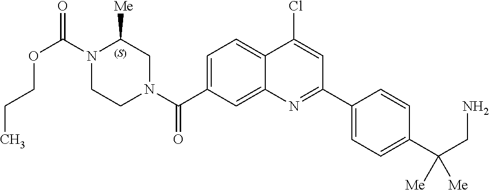 | |
| E019 | 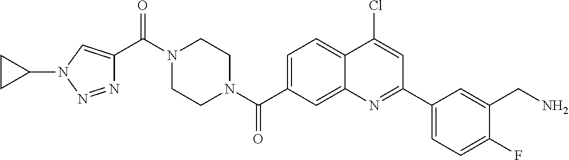 [SMILES: NCc1cc(-c2cc(Cl)c3ccc(C(=O)N4CCN(C(=O)c5cn(C6CC6)nn5)CC4)cc3n2)ccc1F] | 0.039 |
| E020 | 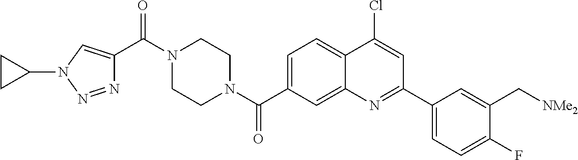 [SMILES: CN(C)Cc1cc(-c2cc(Cl)c3ccc(C(=O)N4CCN(C(=O)c5cn(C6CC6)nn5)CC4)cc3n2)ccc1F] | 0.058 |

### Example 8

Characterization Data

### Propyl 4-(9-chloro-5,6,7,8-tetrahydroacridine-3-carbonyl)piperazine-1-carboxylate (A001)

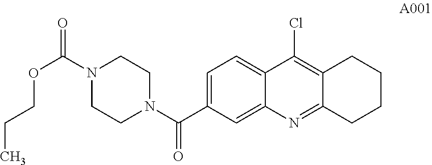 [SMILES: CCCOC(=O)N1CCN(C(=O)c2ccc3c(Cl)c4c(nc3c2)CCCC4)CC1]

Compound A001 was prepared according to General Procedure C1, using commercially available 9-chloro-5,6,7,8-tetrahydroacridine-3-carboxylic acid and n-propyl piperazine-1-carboxylate as starting materials.

¹H NMR (400 MHz, CDCl₃) δ 8.19 (d, J=8.6 Hz, 1H), 7.94 (d, J=1.2 Hz, 1H), 7.65 (dd, J=8.6, 1.5 Hz, 1H), 3.97 (t, J=6.6 Hz, 2H), 3.80-3.28 (m, 8H), 3.06 (s, 2H), 2.99 (s, 2H), 1.90 (s, 4H), 1.66-1.47 (m, 2H), 0.89 (t, J=7.3 Hz, 3H).

LCMS (ESI-TOF) m/z 416.1 [M+H⁺] with a purity of >95%.

### Allyl 4-(9-chloro-5,6,7,8-tetrahydroacridine-3-carbonyl)piperazine-a1-carboxylate (A002)

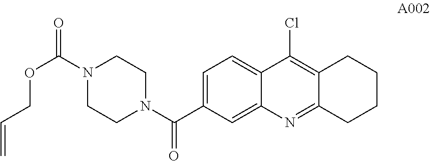 [SMILES: C=CCOC(=O)N1CCN(C(=O)c2ccc3c(Cl)c4c(nc3c2)CCCC4)CC1]

Compound A002 was prepared according to General Procedure C1, step 3a, using commercially available 9-chloro-5,6,7,8-tetrahydroacridine-3-carboxylic acid and allyl piperazine-1-carboxylate as starting materials.

¹H NMR (400 MHz, CD₃OD) δ 8.41 (d, J=8.6 Hz, 1H), 8.02 (d, J=1.1 Hz, 1H), 7.76 (dd, J=8.7, 1.5 Hz, 1H), 6.05-5.82 (m, 1H), 5.31 (d, J=17.6 Hz, 1H), 5.21 (d, J=10.4 Hz, 1H), 4.61 (d, J=5.5 Hz, 2H), 3.99-3.44 (m, 2H), 3.20 (s, 2H), 3.11 (s, 2H), 2.02 (dd, J=6.3, 3.0 Hz, 4H).

LCMS (ESI-TOF) m/z 414.1 [M+H⁺] with a purity of >95%.

### 1-(4-(9-Chloro-5,6,7,8-tetrahydroacridine-3-carbonyl)piperazin-1-yl)pentan-1-one (A003)

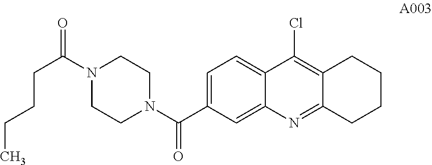 [SMILES: CCCCC(=O)N1CCN(C(=O)c2ccc3c(Cl)c4c(nc3c2)CCCC4)CC1]

Compound A003 was prepared according to General Procedure C1 using (9-chloro-5,6,7,8-tetrahydroacridin-3-yl)(piperazin-1-yl)methanone and valeric acid as starting materials.

¹H NMR (400 MHz, DMSO-d₆) δ 8.22 (d, J=8.6 Hz, 1H), 7.97 (d, J=1.2 Hz, 1H), 7.68 (dd, J=8.6, 1.5 Hz, 1H), 3.83-3.45 (m, 8H), 3.08 (s, 2H), 3.00 (s, 2H), 2.34 (br s, 2H), 1.91 (s, 4H), 1.54-1.43 (m, 2H), 1.40-1.25 (m, 2H), 0.88 (t, J=7.3 Hz, 3H).

LCMS (ESI-TOF) m/z 414.1 [M+H⁺] with a purity of >96%.

### Propyl 8-(9-chloro-5,6,7,8-tetrahydroacridine-3-carbonyl)-3,8-diazabicyclo[3.2.1]octane-3-carboxylate (A004)

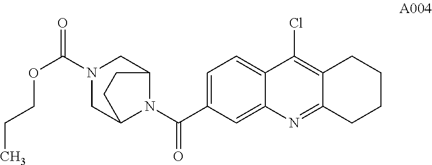 [SMILES: CCCOC(=O)N1CC2CCC(C1)N2C(=O)c1ccc2c(Cl)c3c(nc2c1)CCCC3]

Step 1: According to General Procedure C1, commercially available 9-chloro-5,6,7,8-tetrahydroacridine-3-carboxylic acid was reacted with tert-butyl 3,8-diazabicyclo[3.2.1]octane-3-carboxylate to give tert-butyl 8-(9-chloro-5,6,7,8-tetrahydroacridine-3-carbonyl)-3,8-diazabicyclo[3.2.1]octane-3-carboxylate.

Step 2: The above product (190 mg, 0.417 mmol) was dissolved in trifluoroacetic acid (0.5 mL) and dichloromethane (8 mL) for 24 h. The mixture was diluted with dichloromethane (50 mL) and the organic layer was washed with saturated sodium bicarbonate, brine, dried over anhydrous sodium sulfate, filtered and concentrated under reduced pressure to give crude (3,8-diazabicyclo[3.2.1]octan-8-yl)(9-chloro-5,6,7,8-tetrahydroacridin-3-yl)methanone.

Step 3: The crude material from above (72 mg, 0.202 mmol) was dissolved in dichloromethane (2.0 mL) and triethylamine (56 μL, 0.405 mmol, 2 equiv) followed by n-propyl chloroformate (34 μL, 0.303 mmol, 1.5 equiv) were added at 0° C. After 1 h, the mixture was diluted with ethyl acetate (50 mL) and the organic layer was washed with saturated bicarbonate, brine, dried over anhydrous sodium sulfate, and concentrated under reduced pressure. The crude material was purified by column chromatography (ethyl acetate/hexanes) to afford A004 as a white solid (10.0 mg, 11%) upon lyophilization.

¹NMR (400 MHz, CDCl₃) δ 8.24 (d, J=8.6 Hz, 1H), 8.04 (d, J=1.2 Hz, 1H), 7.67 (dd, J=8.6, 1.4 Hz, 1H), 4.88 (s, 1H), 4.24-3.65 (m, 5H), 3.40-2.93 (m, 6H), 2.13-1.89 (m, 5H), 1.81 (s, 2H), 1.66 (d, J=6.2 Hz, 2H), 1.01-0.76 (m, 4H).

LCMS (ESI-TOF) m/z 442.2 [M+H⁺] with a purity of >99%.

### Propyl 5-(9-chloro-5,6,7,8-tetrahydroacridine-3-carbonyl)-2,5-diazabicyclo[2.2.2]octane-2-carboxylate (A005)

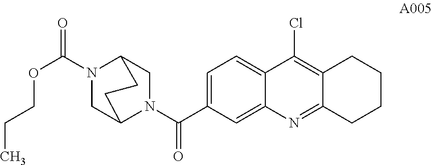 [SMILES: CCCOC(=O)N1CC2CCC1CN2C(=O)c1ccc2c(Cl)c3c(nc2c1)CCCC3]

Step 1: According to General Procedure C1, commercially available 9-chloro-5,6,7,8-tetrahydroacridine-3-carboxylic acid was reacted with tert-butyl 2,5-diazabicyclo[2.2.2]octane-2-carboxylate to afford tert-butyl 5-(9-chloro-5,6,7,8-tetrahydroacridine-3-carbonyl)-2,5-diazabicyclo[2.2.2]octane-2-carboxylate.

Step 2: The above intermediate (120 mg, 0.263 mmol) was dissolved in trifluoroacetic acid (0.4 mL) and dichloromethane (8 mL) for 24 h. The mixture was diluted with dichloromethane (50 mL) and the organic layer was washed with saturated sodium bicarbonate, brine, dried over anhydrous sodium sulfate, filtered and concentrated under reduced pressure to give crude (2,5-diazabicyclo[2.2.2]octan-2-yl)(9-chloro-5,6,7,8-tetrahydroacridin-3-yl)methanone.

Step 3: The crude material (70 mg, 0.197 mmol) was dissolved in dichloromethane (2.0 mL) and triethylamine (39.8 mg, 0.393 mmol, 2 equiv) followed by n-propyl chloroformate (36.2 mg, 0.295 mmol, 1.5 equiv) were added at 0° C. After 1 h, the mixture was diluted with ethyl acetate (50 mL) and the organic layer was washed with saturated bicarbonate, brine, dried over anhydrous sodium sulfate, and concentrated under reduced pressure. The crude material was purified by column chromatography (ethyl acetate/hexanes) to afford A005 as a white solid (10.0 mg, 11%) upon lyophilization.

¹NMR (400 MHz, CDCl₃) δ 8.24 (d, J=8.3 Hz, 1H), 8.05-7.91 (m, 1H), 7.69-7.54 (m, 1H), 4.90, 4.54 and 4.42 (multiple peaks, 1H), 4.28-3.89 (m, 3H), 3.85-3.64 (m, 2H), 3.64-3.34 (m, 2H), 3.14 (s, 2H), 3.04-2.87 (m, 2H), 2.24-1.84 (m, 6H), 1.84-1.62 (m, 2H), 1.09-0.72 (m, 5H).

LCMS (ESI-TOF) m/z 442.1 [M+H⁺] with a purity of >99%.

### (9-Chloro-5,6,7,8-tetrahydroacridin-3-yl)(4-(5-cyclopropylisoxazole-3-carbonyl)piperazin-1-yl)methanone (A006)

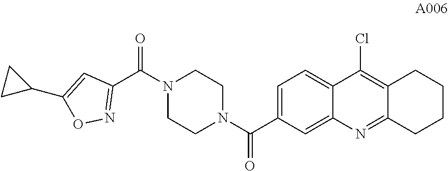 [SMILES: O=C(c1ccc2c(Cl)c3c(nc2c1)CCCC3)N1CCN(C(=O)c2cc(C3CC3)on2)CC1]

Compound A006 was prepared according to General Procedure C1 using (9-chloro-5,6,7,8-tetrahydroacridin-3-yl)(piperazin-1-yl)methanone and 5-cyclopropylisoxazole-3-carboxylic acid as starting materials.

¹H NMR (400 MHz, DMSO) δ 8.22 (d, J=8.5 Hz, 1H), 7.99 (s, 1H), 7.69 (d, J=8.5 Hz, 1H), 6.44 (s, 1H), 3.71 (s, 8H), 3.08 (s, 2H), 3.00 (s, 2H), 2.20 (s, 1H), 1.91 (s, 4H), 1.09 (s, 2H), 0.94 (s, 2H).

LCMS (ESI-TOF) m/z 465.1 [M+H⁺] with a purity of >95%.

### (9-Chloro-5,6,7,8-tetrahydroacridin-3-yl)(4-(5-isobutylisoxazole-3-carbonyl)piperazin-1-yl)methanone (A007)

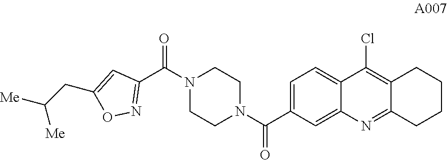 [SMILES: CC(C)Cc1cc(C(=O)N2CCN(C(=O)c3ccc4c(Cl)c5c(nc4c3)CCCC5)CC2)no1]

Compound A007 was prepared according to General Procedure C1 using (9-chloro-5,6,7,8-tetrahydroacridin-3-yl)(piperazin-1-yl)methanone and 5-isobutylisoxazole-3-carboxylic acid as starting materials.

¹H NMR (400 MHz, DMSO-d₆) δ 8.22 (d, J=8.6 Hz, 1H), 7.99 (s, 1H), 7.69 (d, J=8.6 Hz, 1H), 6.52 (s, 1H), 3.72 (br s, 8H), 3.07 (s, 2H), 3.00 (s, 2H), 2.70 (s, 2H), 2.00 (br s, 1H), 1.91 (s, 4H), 0.93 (d, J=6.1 Hz, 6H).

LCMS (ESI-TOF) m/z 481.2 [M+H⁺] with a purity of >97%.

### Propyl 4-(9-chloro-7-methyl-5,6,7,8-tetrahydroacridine-3-carbonyl)piperazine-1-carboxylate

(A008)

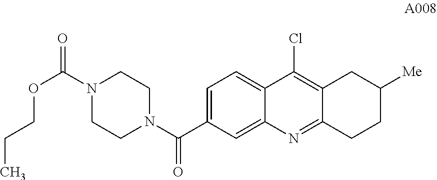 [SMILES: CCCOC(=O)N1CCN(C(=O)c2ccc3c(Cl)c4c(nc3c2)CCC(C)C4)CC1]

Compound A008 was prepared according to General Procedure A, B and C2 using 4-methylcyclohexanone (General Procedure A) and n-propyl piperazine-1-carboxylate (General Procedure C2) as starting materials.

¹H NMR (400 MHz, DMSO-d₆) δ 8.19 (d, J=8.8 Hz, 1H), 7.94 (s, 1H), 7.65 (d, J=8.8, 1.6 Hz, 1H), 3.98 (t, J=6.4 Hz, 2H), 3.66-3.31 (m, 8H), 3.26-3.07 (m, 3H), 2.47-2.46 (m, 1H), 2.00-1.97 (m, 2H), 1.60-1.52 (m, 3H), 1.14 (d, J=6.4 Hz, 3H), 0.89 (t, J=7.6 Hz, 3H).

LCMS (ESI-TOF) m/z 430.2 [M+H⁺] with a purity of >96%.

### Propyl 3-(9-chloro-5,6,7,8-tetrahydroacridine-3-carbonyl)-3,8-diazabicyclo[3.2.1]octane-8-carboxylate (A009)

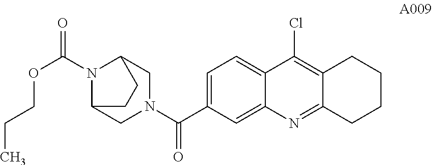 [SMILES: CCCOC(=O)N1C2CCC1CN(C(=O)c1ccc3c(Cl)c4c(nc3c1)CCCC4)C2]

Step 1: According to General Procedure C1, commercially available 9-chloro-5,6,7,8-tetrahydroacridine-3-carboxylic acid was reacted with tert-butyl 3,8-diazabicyclo[3.2.1]octane-8-carboxylate to give tert-butyl 3-(9-chloro-5,6,7,8-tetrahydroacridine-3-carbonyl)-3,8-diazabicyclo[3.2.1]octane-8-carboxylate.

Step 2: The resulting intermediate (220 mg, 0.482 mmol) was dissolved in trifluoroacetic acid (1 mL) and dichloromethane (10 mL) for 24 h. The mixture was diluted with dichloromethane (50 mL) and the organic layer was washed with saturated sodium bicarbonate, brine, dried over anhydrous sodium sulfate, filtered and concentrated under reduced pressure to give crude (3,8-diazabicyclo[3.2.1]octan-3-yl)(9-chloro-5,6,7,8-tetrahydroacridin-3-yl)methanone.

Step 3: The crude material from above (190 mg, 0.534 mmol) was dissolved in dichloromethane (5.0 mL) and triethylamine (58.9 mg, 0.582 mmol, 1.09 equiv) followed by n-propyl chloroformate (53.5 mg, 0.436 mmol, 0.82 equiv) were added at 0° C. After 1 h, the mixture was diluted with ethyl acetate (50 mL) and the organic layer was washed with saturated bicarbonate, brine, dried over anhydrous sodium sulfate, and concentrated under reduced pressure. The crude material was purified by column chromatography (ethyl acetate/hexanes) to afford A009 as a white solid (100 mg, 42%) upon lyophilization.

¹H NMR (400 MHz, 80° C., DMSO-d₆) δ 8.19 (d, J=8.6 Hz, 1H), 7.91 (d, J=1.2 Hz, 1H), 7.64 (dd, J=8.6, 1.5 Hz, 1H), 4.47-4.23 (m, 2H), 4.09 (s, 1H), 3.99 (t, J=6.5 Hz, 2H), 3.45-3.33 (m, 2H), 3.09-2.93 (m, 5H), 1.90-1.71 (m, 7H), 1.59 (dd, J=13.8, 6.7 Hz, 3H), 0.89 (t, J=7.3 Hz, 3H).

LCMS (ESI-TOF) m/z 442.2 [M+H⁺] with a purity of >99%.

### Propyl (S)-4-(9-chloro-5,6,7,8-tetrahydroacridine-3-carbonyl)-3-methylpiperazine-1-carboxylate (A010)

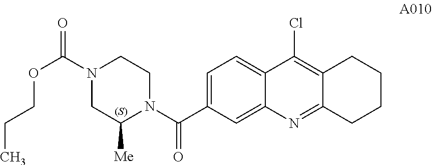

Compound A010 was prepared according to General Procedure C1, using commercially available 9-chloro-5,6,7,8-tetrahydroacridine-3-carboxylic acid and (S)-n-propyl 3-methylpiperazine-1-carboxylate as starting materials.

¹NMR (400 MHz, DMSO-d₆) δ 8.20 (d, J=8.6 Hz, 1H), 7.91 (d, J=1.2 Hz, 1H), 7.63 (dd, J=8.6, 1.5 Hz, 1H), 4.33-3.64 (m, 6H), 3.25-2.85 (m, 7H), 1.90 (s, 4H), 1.58 (dq, J=14.3, 7.1 Hz, 2H), 1.16 (d, J=4.7 Hz, 3H), 0.89 (t, J=7.3 Hz, 3H).

LCMS (ESI-TOF) m/z 430.1 [M+H⁺] with a purity of >96%.

### Propyl (R)-4-(9-chloro-5,6,7,8-tetrahydroacridine-3-carbonyl)-3-methylpiperazine-1-carboxylate (A011)

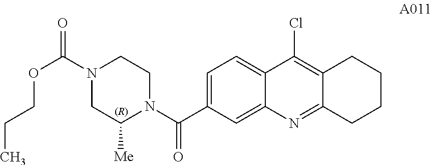

Compound A011 was prepared according to General Procedure C1, using commercially available 9-chloro-5,6,7,8-tetrahydroacridine-3-carboxylic acid and (R)-n-propyl 3-methylpiperazine-1-carboxylate as starting materials.

¹NMR (400 MHz, DMSO-d₆) δ 8.20 (d, J=8.6 Hz, 1H), 7.91 (d, J=1.2 Hz, 1H), 7.63 (dd, J=8.6, 1.5 Hz, 1H), 4.34-3.66 (m, 6H), 3.26-2.84 (m, 7H), 1.90 (s, 4H), 1.63-1.49 (m, 2H), 1.16 (d, J=5.1 Hz, 3H), 0.89 (t, J=7.4 Hz, 3H).

LCMS (ESI-TOF) m/z 430.1 [M+H⁺] with a purity of >97%.

### (9-Chloro-5,6,7,8-tetrahydroacridin-3-yl)(4-(5-methylisoxazole-3-carbonyl)piperazin-1-yl)methanone (A012)

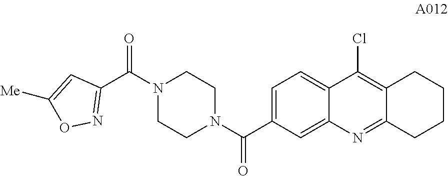 [SMILES: Cc1cc(C(=O)N2CCN(C(=O)c3ccc4c(Cl)c5c(nc4c3)CCCC5)CC2)no1]

Compound A012 was prepared according to General Procedure C1 using (9-chloro-5,6,7,8-tetrahydroacridin-3-yl)(piperazin-1-yl)methanone and 5-methylisoxazole-3-carboxylic acid as starting materials.

¹NMR (400 MHz, DMSO-d₆) δ 8.22 (d, J=8.8 Hz, 1H), 7.99 (s, 1H), 7.70 (d, J=9.9 Hz, 1H), 6.48 (s, 1H), 3.71 (br s, 8H), 3.08 (s, 2H), 3.00 (s, 2H), 2.46 (s, 3H), 1.91 (s, 4H).

LCMS (ESI-TOF) m/z 439.1 [M+H⁺] with a purity of >96%.

### Propyl 3-(9-chloro-5,6,7,8-tetrahydroacridine-3-carbonyl)-3,9-diazabicyclo[3.3.1]nonane-9-carboxylate (A013)

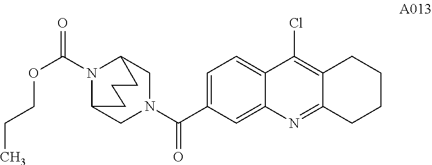 [SMILES: CCCOC(=O)N1C2CCCC1CN(C(=O)c1ccc3c(Cl)c4c(nc3c1)CCCC4)C2]

Step 1: According to General Procedure C1, commercially available 9-chloro-5,6,7,8-tetrahydroacridine-3-carboxylic acid was reacted with tert-butyl 7,9-diazabicyclo[3.3.1]nonane-9-carboxylate to afford tert-butyl 3-(9-chloro-5,6,7,8-tetrahydroacridine-3-carbonyl)-3,9-diazabicyclo[3.3.1]nonane-9-carboxylate.

Step 2: The resulting intermediate (230 mg, 0.489 mmol) was dissolved in trifluoroacetic acid (1 mL) and dichloromethane (10 mL) for 24 h. The mixture was diluted with dichloromethane (50 mL) and the organic layer was washed with saturated sodium bicarbonate, brine, dried over anhydrous sodium sulfate, filtered and concentrated under reduced pressure to afford (3,9-diazabicyclo[3.3.1]nonan-3-yl)(9-chloro-5,6,7,8-tetrahydroacridin-3-yl)methanone.

Step 3: The crude material from above (100 mg, 0.27 mmol) was dissolved in dichloromethane (5.0 mL) and triethylamine (58.9 mg, 0.582 mmol, 2.15 equiv) followed by n-propyl chloroformate (53.5 mg, 0.436 mmol, 1.61 equiv) were added at 0° C. After 1 h, the mixture was diluted with ethyl acetate (50 mL) and the organic layer was washed with saturated sodium bicarbonate, brine, dried over anhydrous sodium sulfate, and concentrated under reduced pressure. The crude material was purified by column chromatography (ethyl acetate/hexanes) to afford A013 as a white solid (50 mg, 41%) upon lyophilization.

¹H NMR (400 MHz, 80° C., DMSO-d₆) δ 8.20 (d, J=8.6 Hz, 1H), 7.92 and 7.86 (2×s, 1H), 7.64 and 7.59 (2×d, J=9.1 and 8.6 Hz, 1H), 4.74-3.71 (m, 6H), 3.45-3.16 (m, 2H), 3.04 (s, 2H), 2.99 (s, 2H), 2.08 (dt, J=18.8, 9.7 Hz, 1H), 1.91 (s, 4H), 1.79-1.50 (m, 7H), 0.90 (t, J=7.4 Hz, 3H).

LCMS (ESI-TOF) m/z 456.2 [M+H⁺] with a purity of >98%.

### Propyl 6-(9-chloro-5,6,7,8-tetrahydroacridine-3-carbonyl)-3,6-diazabicyclo[3.1.1]heptane-3-carboxylate (A014)

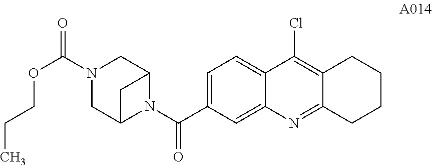 [SMILES: CCCOC(=O)N1CC2CC(C1)N2C(=O)c1ccc2c(Cl)c3c(nc2c1)CCCC3]

Step 1: According to General Procedure C1, commercially available 9-chloro-5,6,7,8-tetrahydroacridine-3-carboxylic acid was reacted with tert-butyl 3,6-diazabicyclo[3.1.1]heptane-3-carboxylate to give tert-butyl 6-(9-chloro-5,6,7,8-tetrahydroacridine-3-carbonyl)-3,6-diazabicyclo[3.1.1]heptane-3-carboxylate

Step 2: The resulting intermediate (200 mg, 0.453 mmol) was dissolved in trifluoroacetic acid (1 mL) and dichloromethane (10 mL) for 24 h. The mixture was diluted with dichloromethane (50 mL) and the organic layer was washed with saturated sodium bicarbonate, brine, dried over anhydrous sodium sulfate, filtered and concentrated under reduced pressure to afford (3,6-diazabicyclo[3.1.1]heptan-6-yl)(9-chloro-5,6,7,8-tetrahydroacridin-3-yl)methanone.

Step 3: The crude material from above (100 mg, 0.293 mmol) was dissolved in dichloromethane (5.0 mL) and triethylamine (58.9 mg, 0.582 mmol, 1.99 equiv) followed by n-propyl chloroformate (53.5 mg, 0.436 mmol, 1.5 equiv) were added at 0° C. After 1 h, the mixture was diluted with ethyl acetate (50 mL) and the organic layer was washed with saturated sodium bicarbonate, brine, dried over anhydrous sodium sulfate, and concentrated under reduced pressure. The crude material was purified by column chromatography (ethyl acetate/hexanes) to afford A014 as a white solid (50 mg, 40%) upon lyophilization.

¹NMR (400 MHz, 80° C., DMSO-d₆) δ 8.20 (d, J=8.7 Hz, 1H), 8.14 (d, J=1.3 Hz, 1H), 7.82 (d, J=8.7 Hz, 1H), 4.71 (s, 1H), 4.51 (s, 1H), 4.15-3.83 (m, 3H), 3.54-3.18 (m, 2H), 3.07 (s, 2H), 2.99 (s, 2H), 2.80 (dd, J=14.7, 6.8 Hz, 1H), 1.90 (s, 4H), 1.69-1.38 (m, 3H), 0.98-0.66 (m, 4H).

LCMS (ESI-TOF) m/z 428.1 [M+H⁺] with a purity of >95%.

### Propyl 9-(9-chloro-5,6,7,8-tetrahydroacridine-3-carbonyl)-3,9-diazabicyclo[3.3.1]nonane-3-carboxylate (A015)

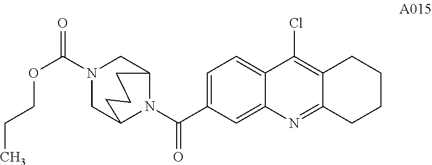 [SMILES: CCCOC(=O)N1CC2CCCC(C1)N2C(=O)c1ccc2c(Cl)c3c(nc2c1)CCCC3]

Step 1: According to General Procedure C1, commercially available 9-chloro-5,6,7,8-tetrahydroacridine-3-carboxylic acid was reacted with tert-butyl 3,9-diazabicyclo[3.3.1]nonane-3-carboxylate to afford tert-butyl 9-(9-chloro-5,6,7,8-tetrahydroacridine-3-carbonyl)-3,9-diazabicyclo[3.3.1]nonane-3-carboxylate.

Step 2: The resulting intermediate (200 mg, 0.426 mmol) was dissolved in trifluoroacetic acid (1 mL) and dichloromethane (10 mL) for 24 h. The mixture was diluted with dichloromethane (50 mL) and the organic layer was washed with saturated sodium bicarbonate, brine, dried over anhydrous sodium sulfate, filtered and concentrated under reduced pressure to give crude (3,9-diazabicyclo[3.3.1]nonan-9-yl)(9-chloro-5,6,7,8-tetrahydroacridin-3-yl)methanone.

Step 3: The crude material from above (100 mg, 0.27 mmol) was dissolved in dichloromethane (5.0 mL) and triethylamine (58.9 mg, 0.582 mmol, 2.15 equiv) followed by n-propyl chloroformate (53.5 mg, 0.436 mmol, 1.61 equiv) were added at 0° C. After 1 h, the mixture was diluted with ethyl acetate (50 mL) and the organic layer was washed with saturated sodium bicarbonate, brine, dried over anhydrous sodium sulfate, and concentrated under reduced pressure. The crude material was purified by column chromatography (ethyl acetate/hexanes) to afford A015 as a white solid (50 mg, 41%) upon lyophilization.

¹NMR (400 MHz, DMSO-d₆) δ 8.20 (d, J=8.6 Hz, 1H), 7.95 (d, J=1.2 Hz, 1H), 7.67 (dd, J=8.6, 1.4 Hz, 1H), 4.66 (s, 1H), 4.16-3.85 (m, 4H), 3.70 (s, 1H), 3.24-3.10 (m, 2H), 3.06 (s, 2H), 2.99 (s, 2H), 1.97-1.69 (m, 8H), 1.68-1.49 (m, 4H), 0.89 (s, 3H).

LCMS (ESI-TOF) m/z 456.2 [M+H⁺] with a purity of >95%.

### Propyl 4-(9-chloro-5,6,7,8-tetrahydroacridine-3-carbonyl)-3-ethylpiperazine-1-carboxylate (A016)

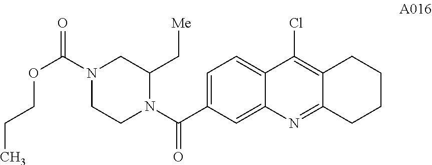 [SMILES: CCCOC(=O)N1CCN(C(=O)c2ccc3c(Cl)c4c(nc3c2)CCCC4)C(CC)C1]

Step 1: According to General Procedure C1, commercially available 9-chloro-5,6,7,8-tetrahydroacridine-3-carboxylic acid was reacted with tert-butyl 3-ethylpiperazine-1-carboxylate to give tert-butyl 4-(9-chloro-5,6,7,8-tetrahydroacridine-3-carbonyl)-3-ethylpiperazine-1-carboxylate

Step 2: The resulting intermediate (200 mg, 0.437 mmol) was dissolved in trifluoroacetic acid (1 mL) and dichloromethane (10 mL) for 24 h. The mixture was diluted with dichloromethane (50 mL) and the organic layer was washed with saturated sodium bicarbonate, brine, dried over anhydrous sodium sulfate, filtered and concentrated under reduced pressure to afford (9-chloro-5,6,7,8-tetrahydroacridin-3-yl)(2-ethylpiperazin-1-yl)methanone.

Step 3: The crude material from above (100 mg, 0.279 mmol) was dissolved in dichloromethane (5.0 mL) and triethylamine (58.9 mg, 0.582 mmol, 2.08 equiv) followed by n-propyl chloroformate (53.5 mg, 0.436 mmol, 1.56 equiv) were added at 0° C. After 1 h, the mixture was diluted with ethyl acetate (50 mL) and the organic layer was washed with saturated sodium bicarbonate, brine, dried over anhydrous sodium sulfate, and concentrated under reduced pressure. The crude material was purified by column chromatography (ethyl acetate/hexanes) to afford A016 as a white solid (50 mg, 40%) upon lyophilization.

¹H NMR (400 MHz, DMSO-d₆) δ 8.20 (d, J=8.6 Hz, 1H), 7.90 (s, 1H), 7.62 (dd, J=8.6, 1.4 Hz, 1H), 4.70-4.21 (m, 1H), 4.10-3.74 (m, 4H), 3.67-3.34 (m, 1H), 3.26-2.79 (m, 7H), 1.90 (s, 4H), 1.76-1.38 (m, 4H), 1.09-0.50 (m, 6H).

LCMS (ESI-TOF) m/z 444.1 [M+H⁺] with a purity of >96%.

### Propyl 4-(9-chloro-5,6,7,8-tetrahydroacridine-3-carbonyl)-2,5-dimethylpiperazine-1-carboxylate (A017)

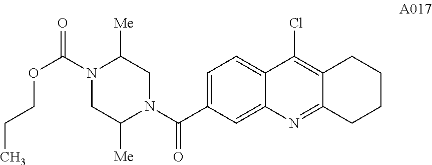 [SMILES: CCCOC(=O)N1CC(C)N(C(=O)c2ccc3c(Cl)c4c(nc3c2)CCCC4)CC1C]

Step 1: According to General Procedure C1, commercially available 9-chloro-5,6,7,8-tetrahydroacridine-3-carboxylic acid was reacted with tert-butyl 2,5-dimethylpiperazine-1-carboxylate to give tert-butyl 4-(9-chloro-5,6,7,8-tetrahydroacridine-3-carbonyl)-2,5-dimethylpiperazine-1-carboxylate

Step 2: The resulting intermediate (200 mg, 0.437 mmol) was dissolved in trifluoroacetic acid (1 mL) and dichloromethane (10 mL) for 24 h. The mixture was diluted with dichloromethane (50 mL) and the organic layer was washed with saturated sodium bicarbonate, brine, dried over anhydrous sodium sulfate, filtered and concentrated under reduced pressure to afford (9-chloro-5,6,7,8-tetrahydroacridin-3-yl)(2,5-dimethylpiperazin-1-yl)methanone.

Step 3: The crude material from above (100 mg, 0.279 mmol) was dissolved in dichloromethane (5.0 mL) and triethylamine (58.9 mg, 0.582 mmol, 2.08 equiv) followed by n-propyl chloroformate (53.5 mg, 0.436 mmol, 1.56 equiv) were added at 0° C. After 1 h, the mixture was diluted with ethyl acetate (50 mL) and the organic layer was washed with saturated sodium bicarbonate, brine, dried over anhydrous sodium sulfate, and concentrated under reduced pressure. The crude material was purified by column chromatography (ethyl acetate/hexanes) to afford A017 as a white solid (50 mg, 40%) upon lyophilization.

¹H NMR (400 MHz, DMSO-d₆) δ 8.24-8.13 (m, 1H), 7.90 (d, J=30.2 Hz, 1H), 7.63 (dd, J=28.3, 8.5 Hz, 1H), 4.79 and 4.39 (2×s, 1H), 4.29-3.88 (m, 3H), 3.88-3.63 (m, 1H), 3.63-3.44 (m, 1H), 3.37-3.17 (m, 2H), 3.06 (s, 2H), 2.98 (s, 2H), 1.90 (s, 4H), 1.58 (d, J=6.9 Hz, 2H), 1.30-0.97 (m, 6H), 0.97-0.77 (m, 3H).

LCMS (ESI-TOF) m/z 444.2 [M+H⁺] with a purity of >96%.

### Propyl 4-(9-chloro-5,6,7,8-tetrahydroacridine-3-carbonyl)-2-methylpiperazine-1-carboxylate (A018)

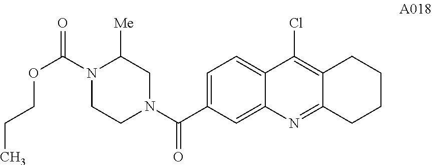 [SMILES: CCCOC(=O)N1CCN(C(=O)c2ccc3c(Cl)c4c(nc3c2)CCCC4)CC1C]

Step 1: According to General Procedure C1, commercially available 9-chloro-5,6,7,8-tetrahydroacridine-3-carboxylic acid was reacted with tert-butyl 2-methylpiperazine-1-carboxylate to give tert-butyl 4-(9-chloro-5,6,7,8-tetrahydroacridine-3-carbonyl)-2-methylpiperazine-1-carboxylate.

Step 2: The resulting intermediate (160 mg, 0.36 mmol) was dissolved in trifluoroacetic acid (1 mL) and dichloromethane (10 mL) for 24 h. The mixture was diluted with dichloromethane (50 mL) and the organic layer was washed with saturated sodium bicarbonate, brine, dried over anhydrous sodium sulfate, filtered and concentrated under reduced pressure to afford (9-chloro-5,6,7,8-tetrahydroacridin-3-yl)(3-methylpiperazin-1-yl)methanone.

Step 3: The crude material from above (100 mg, 0.291 mmol) was dissolved in dichloromethane (5.0 mL) and triethylamine (58.9 mg, 0.582 mmol, 2.00 equiv) followed by n-propyl chloroformate (53.5 mg, 0.436 mmol, 1.50 equiv) were added at 0° C. After 1 h, the mixture was diluted with ethyl acetate (50 mL) and the organic layer was washed with saturated sodium bicarbonate, brine, dried over anhydrous sodium sulfate, and concentrated under reduced pressure. The crude material was purified by column chromatography (ethyl acetate/hexanes) to afford A018 as a white solid (50 mg, 40%) upon lyophilization.

¹H NMR (400 MHz, DMSO-d₆) δ 8.20 (d, J=8.6 Hz, 1H), 7.92 (s, 1H), 7.65 (d, J=5.5 Hz, 1H), 4.58-4.04 (m, 2H), 3.97 (tt, J=10.6, 5.3 Hz, 2H), 3.91-3.32 (m, 2H), 3.23-2.85 (m, 7H), 1.89 (t, J=2.8 Hz, 4H), 1.65-1.48 (m, 2H), 1.25-0.93 (m, 3H), 0.89 (t, J=7.4 Hz, 3H).

LCMS (ESI-TOF) m/z 430.1 [M+H⁺] with a purity of >97%.

### Propyl 3-(9-chloro-5,6,7,8-tetrahydroacridine-3-carbonyl)-3,6-diazabicyclo[3.1.1]heptane-6-carboxylate (A019)

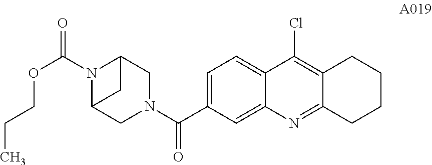 [SMILES: CCCOC(=O)N1C2CC1CN(C(=O)c1ccc3c(Cl)c4c(nc3c1)CCCC4)C2]

Step 1: According to General Procedure C1, commercially available 9-chloro-5,6,7,8-tetrahydroacridine-3-carboxylic acid was reacted with tert-butyl-3,6-diazabicyclo[3.1.1]heptane-6-carboxylate to give tert-butyl 3-(9-chloro-5,6,7,8-tetrahydroacridine-3-carbonyl)-3,6-diazabicyclo[3.1.1]heptane-6-carboxylate.

Step 2: The resulting intermediate (160 mg, 0.362 mmol) was dissolved in trifluoroacetic acid (1 mL) and dichloromethane (10 mL) for 24 h. The mixture was diluted with dichloromethane (50 mL) and the organic layer was washed with saturated sodium bicarbonate, brine, dried over anhydrous sodium sulfate, filtered and concentrated under reduced pressure.to afford (3,6-diazabicyclo[3.1.1]heptan-3-yl)(9-chloro-5,6,7,8-tetrahydroacridin-3-yl)methanone.

Step 3: The crude material from above (100 mg, 0.293 mmol) was dissolved in dichloromethane (5.0 mL) and triethylamine (58.9 mg, 0.582 mmol, 1.99 equiv) followed by n-propyl chloroformate (53.5 mg, 0.436 mmol, 1.49 equiv) were added at 0° C. After 1 h, the mixture was diluted with ethyl acetate (50 mL) and the organic layer was washed with saturated sodium bicarbonate, brine, dried over anhydrous sodium sulfate, and concentrated under reduced pressure. The crude material was purified by column chromatography (ethyl acetate/hexanes) to afford A019 as a white solid (50 mg, 40%) upon lyophilization.

¹H NMR (400 MHz, DMSO-d₆) δ 8.20 (d, J=8.6 Hz, 1H), 7.93 (s, 1H), 7.61 (d, J=8.4 Hz, 1H), 4.26 (s, 1H), 4.20-3.90 (m, 4H), 3.76 (s, 1H), 3.62 (d, J=13.1 Hz, 1H), 3.51 (s, 1H), 3.06 (s, 2H), 2.99 (s, 2H), 2.60-2.54 (m, 1H), 1.90 (s, 4H), 1.56 (d, J=8.8 Hz, 3H), 0.85 (s, 3H).

LCMS (ESI-TOF) m/z 428.1 [M+H⁺] with a purity of >95%.

### Propyl 4-(9-chloro-5,6,7,8-tetrahydroacridine-3-carbonyl)-2,6-dimethylpiperazine-1-carboxylate (A020)

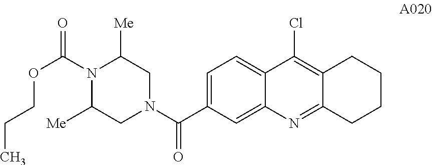 [SMILES: CCCOC(=O)N1C(C)CN(C(=O)c2ccc3c(Cl)c4c(nc3c2)CCCC4)CC1C]

Step 1: According to General Procedure C1, commercially available 9-chloro-5,6,7,8-tetrahydroacridine-3-carboxylic acid was reacted with tert-butyl 2,6-dimethylpiperazine-1-carboxylate to give tert-butyl 4-(9-chloro-5,6,7,8-tetrahydroacridine-3-carbonyl)-2,6-dimethylpiperazine-1-carboxylate.

Step 2: The resulting intermediate (160 mg, 0.349 mmol) was dissolved in trifluoroacetic acid (1 mL) and dichloromethane (10 mL) for 24 h. The mixture was diluted with dichloromethane (50 mL) and the organic layer was washed with saturated sodium bicarbonate, brine, dried over anhydrous sodium sulfate, filtered and concentrated under reduced pressure to give (9-chloro-5,6,7,8-tetrahydroacridin-3-yl) (3,5-dimethylpiperazin-1-yl)methanone.

Step 3: The crude material from above (100 mg, 0.279 mmol) was dissolved in dichloromethane (5.0 mL) and triethylamine (58.9 mg, 0.582 mmol, 2.08 equiv) followed by n-propyl chloroformate (53.5 mg, 0.436 mmol, 1.56 equiv) were added at 0° C. After 1 h, the mixture was diluted with ethyl acetate (50 mL) and the organic layer was washed with saturated sodium bicarbonate, brine, dried over anhydrous sodium sulfate, and concentrated under reduced pressure. The crude material was purified by column chromatography (ethyl acetate/hexanes) to afford A020 as a white solid (20 mg, 16%) upon lyophilization.

¹H NMR (400 MHz, DMSO-d₆) δ 8.21 (d, J=8.6 Hz, 1H), 7.94 (d, J=1.2 Hz, 1H), 7.67 (dd, J=8.6, 1.6 Hz, 1H), 4.48-4.11 (m, 2H), 3.98 (t, J=6.5 Hz, 2H), 3.41 (s, 2H), 3.06 (s, 2H), 2.99 (s, 2H), 1.90 (s, 4H), 1.66-1.50 (m, 2H), 1.32-0.93 (m, 6H), 0.89 (t, J=7.4 Hz, 3H).

LCMS (ESI-TOF) m/z 444.1 [M+H⁺] with a purity of >95%.

### Isobutyl 4-(9-chloro-5,6,7,8-tetrahydroacridine-3-carbonyl)piperazine-1-carboxylate (A021)

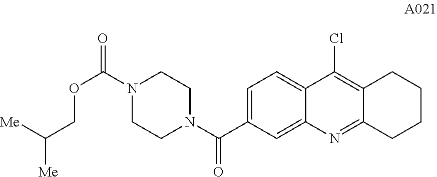 [SMILES: CC(C)COC(=O)N1CCN(C(=O)c2ccc3c(Cl)c4c(nc3c2)CCCC4)CC1]

To a solution of (9-chloro-5,6,7,8-tetrahydroacridin-3-yl)(piperazin-1-yl)methanone (66 mg, 0.2 mmol) in N,N-dimethylformamide (10 mL) at 4° C. was added N,N-diisopropylethylamine (0.35 mL, 2.0 mmol, 10 equiv) and isobutyl chloroformate (0.13 mL, 1.0 mmol, 5 equiv). The resulting mixture was stirred for 2 h at 4° C. followed by 4 h at room temperature. The mixture was quenched with brine and extracted with ethyl acetate. The organic layer was dried over anhydrous sodium sulfate, filtered and concentrated under reduced pressure. The crude material was purified by preparative-HPLC to afford A021 (4.8 mg, 6%) as a yellow oil.

¹H NMR (400 MHz, DMSO-d₆) δ 8.21 (d, J=8.6 Hz, 1H), 7.96 (d, J=1.2 Hz, 1H), 7.67 (dd, J=8.6, 1.6 Hz, 1H), 3.82 (d, J=6.5 Hz, 2H), 3.76-3.44 (m, 8H), 3.07 (s, 2H), 3.00 (s, 2H), 1.91 (s, 5H), 0.90 (d, J=6.6 Hz, 6H).

LCMS (ESI-TOF) m/z 430.1 [M+H⁺] with a purity of >96%.

### Propyl 4-(9-chloro-6-methyl-5,6,7,8-tetrahydroacridine-3-carbonyl)piperazine-1-carboxylate (A022)

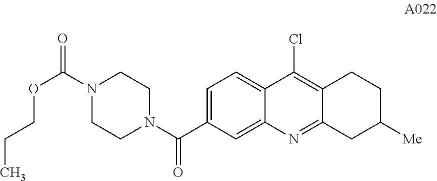 [SMILES: CCCOC(=O)N1CCN(C(=O)c2ccc3c(Cl)c4c(nc3c2)CC(C)CC4)CC1]

Compound A022 was prepared according to General Procedure A, B and C2 using 3-methylcyclohexanone (General Procedure A) and n-propyl piperazine-1-carboxylate (General Procedure C2) as starting materials.

¹H NMR (400 MHz, DMSO-d₆) δ 8.19 (d, J=8.4 Hz, 1H), 7.94 (s, 1H), 7.65 (dd, J=8.4, 1.6 Hz, 1H), 3.96 (t, J=6.4 Hz, 2H), 3.66-3.21 (m, 8H), 3.15-3.11 (m, 2H), 2.94-2.87 (m, 1H), 2.72-2.65 (m, 1H), 2.00 (br s, 2H), 1.60-1.48 (m, 3H), 1.00 (d, J=6.4 Hz, 3H), 0.89 (t, J=7.2 Hz, 3H).

LCMS (ESI-TOF) m/z 430.8 [M+H⁺] with a purity of >98%.

### Propyl 4-(9-chloro-8-methyl-5,6,7,8-tetrahydroacridine-3-carbonyl)piperazine-1-carboxylate (A023)

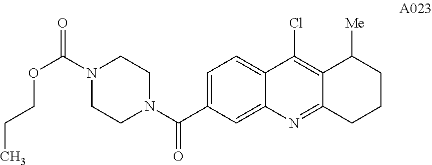 [SMILES: CCCOC(=O)N1CCN(C(=O)c2ccc3c(Cl)c4c(nc3c2)CCCC4C)CC1]

Compound A023 was prepared according to General Procedure A, B and C2 using 3-methylcyclohexanone (General Procedure A) and n-propyl piperazine-1-carboxylate (General Procedure C2) as starting materials.

¹H NMR (400 MHz, DMSO-d₆) δ 8.21 (d, J=8.0 Hz, 1H), 7.93 (s, 1H), 7.65 (dd, J=8.4, 1.6 Hz, 1H), 3.96 (t, J=6.4 Hz, 2H), 3.66-3.18 (m, 9H), 3.17-3.03 (m, 1H), 3.01-2.93 (m, 1H), 2.06-1.99 (m, 4H), 1.60-1.55 (m, 2H), 1.28 (d, J=6.8 Hz, 3H), 0.89 (t, J=7.6 Hz, 3H).

LCMS (ESI-TOF) m/z 430.8 [M+H⁺] with a purity of >97%.

### Propyl 4-(9-chloro-6-phenyl-5,6,7,8-tetrahydroacridine-3-carbonyl)piperazine-1-carboxylate (A024)

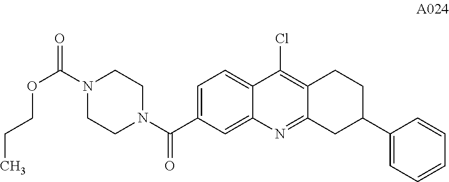 [SMILES: CCCOC(=O)N1CCN(C(=O)c2ccc3c(Cl)c4c(nc3c2)CC(c2ccccc2)CC4)CC1]

Compound A024 was prepared according to General Procedure A, B, and C2 using 3-phenylcyclohexanone (General Procedure A) and n-propyl piperazine-1-carboxylate (General Procedure C2) as starting materials.

¹H NMR (400 MHz, DMSO-d₆) δ 8.22 (d, J=8.8 Hz, 1H), 7.98 (s, 1H), 7.68 (dd, J=8.4, 1.6 Hz, 1H), 7.38-7.33 (m, 4H), 7.27-7.23 (m, 1H), 3.98 (t, J=6.4 Hz, 2H), 3.66-3.28 (m, 8H), 3.26-3.18 (m, 4H), 3.09-3.02 (m, 1H), 2.22-2.19 (m, 1H), 2.08-2.04 (m, 1H), 1.69-1.55 (m, 2H), 0.89 (t, J=7.2 Hz, 3H).

LCMS (ESI-TOF) m/z 492.2 [M+H⁺] with a purity of >98%.

### Propyl 4-(9-chloro-6-cyano-5,6,7,8-tetrahydroacridine-3-carbonyl)piperazine-1-carboxylate (A025)

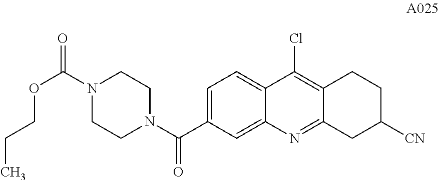 [SMILES: CCCOC(=O)N1CCN(C(=O)c2ccc3c(Cl)c4c(nc3c2)CC(C#N)CC4)CC1]

Compound A025 was prepared according to the General Procedure A, B, and C2 using 2-(3-oxocyclohexyl)acetonitrile (General Procedure A) and n-propyl piperazine-1-carboxylate (General Procedure C2) as starting materials.

¹H NMR (400 MHz, DMSO-d₆) δ 8.23 (d, J=8.0 Hz, 1H), 7.99 (d, J=0.8 Hz, 1H), 7.70 (dd, J=1.2, 8.4 Hz, 1H), 3.97 (t, J=6.8 Hz, 2H), 3.75-3.28 (m, 11H), 3.09 (t, J=6.6 Hz, 2H), 2.30-2.15 (m, 2H), 1.65-1.50 (m, 2H), 0.89 (t, J=7.4 Hz, 3H).

LCMS (ESI-TOF) m/z 441.2 [M+H⁺] with a purity of >96%.

### Propyl 4-(9-chloro-8-cyano-5,6,7,8-tetrahydroacridine-3-carbonyl)piperazine-1-carboxylate (A026)

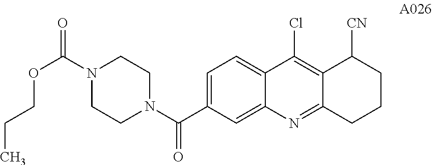 [SMILES: CCCOC(=O)N1CCN(C(=O)c2ccc3c(Cl)c4c(nc3c2)CCCC4C#N)CC1]

Compound A026 was prepared according to General Procedure A, B and C2 using 3-oxocyclohexanecarbonitrile (General Procedure A) and n-propyl piperazine-1-carboxylate (General Procedure C2) as starting materials.

¹H NMR (400 MHz, DMSO-d₆) δ 8.28 (d, J=8.8 Hz, 1H), 8.01 (d, J=0.8 Hz, 1H), 7.73 (dd, J=1.2, 8.4 Hz, 1H), 4.77 (d, J=2.8 Hz, 1H), 3.98 (t, J=6.6 Hz, 2H), 3.75-3.00 (m, 10H), 2.40-1.90 (m, 4H), 1.65-1.50 (m, 2H), 0.89 (t, J=7.2 Hz, 3H).

LCMS (ESI-TOF) m/z 441.2 [M+H⁺] with a purity of >95%.

### Propyl 4-(9-chloro-6-(pyridin-3-yl)-5,6,7,8-tetrahydroacridine-3-carbonyl)piperazine-1-carboxylate (A027)

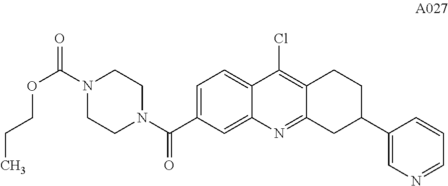 [SMILES: CCCOC(=O)N1CCN(C(=O)c2ccc3c(Cl)c4c(nc3c2)CC(c2cccnc2)CC4)CC1]

Compound A027 was prepared according to General Procedure A, B, and C2 using 3-(pyridin-3-yl)cyclohexanone (General Procedure A) and n-propyl piperazine-1-carboxylate (General Procedure C2) as starting materials.

¹H NMR (400 MHz, DMSO-d₆) δ 8.62 (br s, 1H), 8.48 (br s, 1H), 8.23 (d, J=8.4 Hz, 1H), 7.98 (s, 1H), 7.80 (d, J=6.4 Hz, 1H), 7.68 (d, J=8.4 Hz, 1H), 7.39 (br s, 1H), 3.97 (br s, 2H), 3.67-3.40 (m, 8H), 3.27-3.22 (m, 4H), 3.05 (br s, 1H), 2.22 (br s, 1H), 2.09 (br s, 1H), 1.59-1.57 (d, J=6.4 Hz, 2H), 0.88 (br s, 3H).

LCMS (ESI-TOF) m/z 493.2 [M+H⁺] with a purity of >95%.

### Propyl 4-(6-(aminomethyl)-9-chloro-5,6,7,8-tetrahydroacridine-3-carbonyl)piperazine-1-carboxylate (A028)

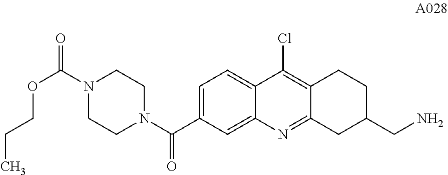 [SMILES: CCCOC(=O)N1CCN(C(=O)c2ccc3c(Cl)c4c(nc3c2)CC(CN)CC4)CC1]

Compound A028 was prepared from A025 as starting material following General Procedure D.

¹H NMR (400 MHz, DMSO-d₆) δ 8.19 (d, J=8.4 Hz, 1H), 7.94 (s, 1H), 7.65 (dd, J=8.8, 1.6 Hz, 1H), 3.98 (t, J=6.4 Hz, 2H), 3.71-3.20 (m, 8H), 3.21-3.11 (m, 2H), 2.91-2.82 (m, 1H), 2.74-2.66 (m, 1H), 2.62-2.61 (m, 2H), 2.10 (br s, 1H), 1.92 (br s, 1H), 1.61-1.47 (m, 3H), 0.89 (t, J=7.2 Hz, 3H).

LCMS (ESI-TOF) m/z 445.2 [M+H⁺] with a purity of >96%.

### Methyl 9-chloro-6-(4-(propoxycarbonyl)piperazine-1-carbonyl)-1,2,3,4-tetrahydroacridine-2-carboxylate (A029)

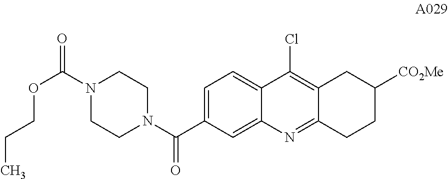 [SMILES: CCCOC(=O)N1CCN(C(=O)c2ccc3c(Cl)c4c(nc3c2)CCC(C(=O)OC)C4)CC1]

Compound A029 was prepared according to General Procedure A, B and C2 using methyl 4-oxocyclohexanecarboxylate (General Procedure A) and n-propyl piperazine-1-carboxylate (General Procedure C2) as starting materials.

¹H NMR (400 MHz, DMSO-d₆) δ8.21 (d, J=8.8 Hz, 1H), 7.95 (s, 1H), 7.67 (d, J=8.8 Hz, 1H), 3.98 (t, J=6.8 Hz, 2H), 3.68-3.60 (m, 5H), 3.50-3.32 (m, 6H), 3.27 (br s, 1H), 3.14-3.08 (m, 4H), 2.27-2.24 (m, 1H), 2.01-1.98 (m, 1H), 1.60-1.55 (m, 2H), 0.89 (t, J=6.8 Hz, 3H).

LCMS (ESI-TOF) m/z 474.27 [M+H⁺] with a purity of >97%.

### Propyl 4-(9-chloro-5,6,7,8-tetrahydroacridine-3-carbonyl)-2-ethylpiperazine-1-carboxylate (A030)

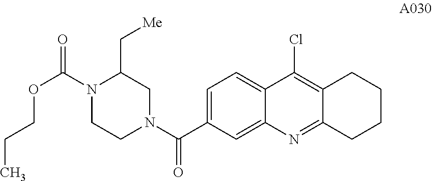 [SMILES: CCCOC(=O)N1CCN(C(=O)c2ccc3c(Cl)c4c(nc3c2)CCCC4)CC1CC]

Step 1: According to General Procedure C1, commercially available 9-chloro-5,6,7,8-tetrahydroacridine-3-carboxylic acid was reacted with tert-butyl 2-ethylpiperazine-1-carboxylate to give tert-butyl 4-(9-chloro-5,6,7,8-tetrahydroacridine-3-carbonyl)-2-ethylpiperazine-1-carboxylate.

Step 2: The resulting intermediate (220 mg, 0.48 mmol) was dissolved in trifluoroacetic acid (1 mL) and dichloromethane (10 mL) for 24 h. The mixture was diluted with dichloromethane (50 mL) and the organic layer was washed with saturated sodium bicarbonate, brine, dried over anhydrous sodium sulfate, filtered and concentrated under reduced pressure to afford (9-chloro-5,6,7,8-tetrahydroacridin-3-yl)(3-ethylpiperazin-1-yl)methanone.

Step 3: The crude material from above (120 mg, 0.335 mmol) was dissolved in dichloromethane (5.0 mL) and triethylamine (58.9 mg, 0.582 mmol, 1.73 equiv) followed by n-propyl chloroformate (53.5 mg, 0.436 mmol, 1.30 equiv) were added at 0° C. After 1 h, the mixture was diluted with ethyl acetate (50 mL) and the organic layer was washed with saturated sodium bicarbonate, brine, dried over anhydrous sodium sulfate, and concentrated under reduced pressure. The crude material was purified by column chromatography (ethyl acetate/hexanes) to afford A030 as a white solid (50 mg, 34%) upon lyophilization.

¹H NMR (400 MHz, DMSO-d₆) δ 8.20 (d, J=8.6 Hz, 1H), 7.92 (s, 1H), 7.64 (d, J=7.9 Hz, 1H), 4.45 (s, 1H), 4.25-3.70 (m, 4H), 3.59-3.36 (m, 1H), 3.22-2.82 (m, 7H), 1.90 (s, 4H), 1.78-1.47 (m, 4H), 0.89 (t, J=7.3 Hz, 5H), 0.56 (s, 1H).

LCMS (ESI-TOF) m/z 444.1 [M+H⁺] with a purity of >98%.

### Propyl 4-(9-chloro-5,6,7,8-tetrahydroacridine-3-carbonyl)-3-methylpiperazine-1-carboxylate (A031)

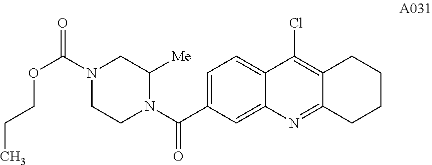 [SMILES: CCCOC(=O)N1CCN(C(=O)c2ccc3c(Cl)c4c(nc3c2)CCCC4)C(C)C1]

Step 1: According to General Procedure C1, commercially available 9-chloro-5,6,7,8-tetrahydroacridine-3-carboxylic acid was reacted with tert-butyl 2-ethylpiperazine-1-carboxylate to afford tert-butyl 4-(9-chloro-5,6,7,8-tetrahydroacridine-3-carbonyl)-3-methylpiperazine-1-carboxylate.

Step 2: The resulting intermediate (220 mg, 0.496 mmol) was dissolved in trifluoroacetic acid (1 mL) and dichloromethane (10 mL) for 24 h. The mixture was diluted with dichloromethane (50 mL) and the organic layer was washed with saturated sodium bicarbonate, brine, dried over anhydrous sodium sulfate, filtered and concentrated under reduced pressure to give (9-chloro-5,6,7,8-tetrahydroacridin-3-yl)(2-methylpiperazin-1-yl)methanone.

Step 3: The crude material from above (120 mg, 0.349 mmol) was dissolved in dichloromethane (5.0 mL) and triethylamine (58.9 mg, 0.582 mmol, 1.73 equiv) followed by n-propyl chloroformate (53.5 mg, 0.436 mmol, 1.25 equiv) were added at 0° C. After 1 h, the mixture was diluted with ethyl acetate (50 mL) and the organic layer was washed with saturated sodium bicarbonate, brine, dried over anhydrous sodium sulfate, and concentrated under reduced pressure. The crude material was purified by column chromatography (ethyl acetate/hexanes) to afford A031 as a white solid (60 mg, 40%) upon lyophilization.

¹H NMR (400 MHz, DMSO-d₆) δ8.20 (d, J=8.6 Hz, 1H), 7.91 (d, J=1.2 Hz, 1H), 7.63 (dd, J=8.6, 1.5 Hz, 1H), 4.96-3.69 (m, 6H), 3.25-2.78 (m, 7H), 1.90 (s, 4H), 1.63-1.45 (m, 2H), 1.16 (d, J=4.7 Hz, 3H), 0.89 (t, J=7.4 Hz, 3H).

LCMS (ESI-TOF) m/z 430.1 [M+H⁺] with a purity of >96%.

### Propyl 4-(9-chloro-5,6,7,8-tetrahydroacridine-3-carbonyl)-2-(hydroxymethyl)piperazine-1-carboxylate (A032)

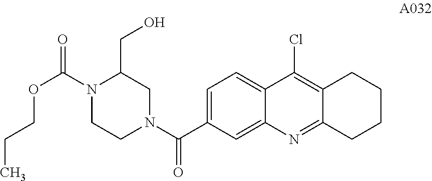 [SMILES: CCCOC(=O)N1CCN(C(=O)c2ccc3c(Cl)c4c(nc3c2)CCCC4)CC1CO]

Step 1: According to General Procedure C1, commercially available 9-chloro-5,6,7,8-tetrahydroacridine-3-carboxylic acid was reacted with tert-butyl-2-(hydroxymethyl)piperazine-1-carboxylate to give tert-butyl 4-(9-chloro-5,6,7,8-tetrahydroacridine-3-carbonyl)-2-(hydroxymethyl)piperazine-1-carboxylate.

Step 2: The resulting intermediate (170 mg, 0.37 mmol) was dissolved in trifluoroacetic acid (1 mL) and dichloromethane (10 mL) for 24 h. The mixture was diluted with dichloromethane (50 mL) and the organic layer was washed with saturated sodium bicarbonate, brine, dried over anhydrous sodium sulfate, filtered and concentrated under reduced pressure to give (9-chloro-5,6,7,8-tetrahydroacridin-3-yl) (3-(hydroxymethyl)piperazin-1-yl)methanone .

Step 3: The crude material from above (120 mg, 0.333 mmol) was dissolved in dichloromethane (5.0 mL) and triethylamine (58.9 mg, 0.582 mmol, 1.74 equiv) followed by n-propyl chloroformate (53.5 mg, 0.436 mmol, 1.31 equiv) were added at 0° C. After 1 h, the mixture was diluted with ethyl acetate (50 mL) and the organic layer was washed with saturated sodium bicarbonate, brine, dried over anhydrous sodium sulfate, and concentrated under reduced pressure. The crude material was purified by column chromatography (ethyl acetate/hexanes) to afford A032 as a white solid (20 mg, 13%) upon lyophilization.

¹H NMR (400 MHz, DMSO-d₆) δ 8.18 (d, J=8.6 Hz, 1H), 7.93 (d, J=1.2 Hz, 1H), 7.64 (d, J=8.6 Hz, 1H), 4.97-4.63 (m, 1H), 4.60-4.27 (m, 1H), 4.26-3.34 (m, 8H), 3.23-2.91 (m, 6H), 1.90 (s, 4H), 1.66-1.49 (m, 2H), 0.89 (t, J=7.4 Hz, 3H).

LCMS (ESI-TOF) m/z 446.1 [M+H⁺] with a purity of >96%.

### Propyl 4-(9-chloro-5,6,7,8-tetrahydroacridine-3-carbonyl)-2,3-dimethylpiperazine-1-carboxylate (A033)

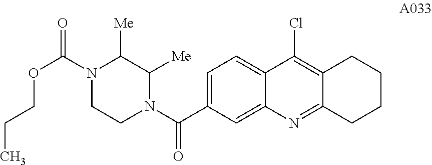 [SMILES: CCCOC(=O)N1CCN(C(=O)c2ccc3c(Cl)c4c(nc3c2)CCCC4)C(C)C1C]

Step 1: According to General Procedure C1, commercially available 9-chloro-5,6,7,8-tetrahydroacridine-3-carboxylic acid was reacted with tert-butyl 2,3-dimethylpiperazine-1-carboxylate to afford tert-butyl 4-(9-chloro-5,6,7,8-tetrahydroacridine-3-carbonyl)-2,3-dimethylpiperazine-1-carboxylate.

Step 2: The resulting intermediate (170 mg, 0.371 mmol) was dissolved in trifluoroacetic acid (1 mL) and dichloromethane (10 mL) for 24 h. The mixture was diluted with dichloromethane (50 mL) and the organic layer was washed with saturated sodium bicarbonate, brine, dried over anhydrous sodium sulfate, filtered and concentrated under reduced pressure to give (9-chloro-5,6,7,8-tetrahydroacridin-3-yl)(2,3-dimethylpiperazin-1-yl)methanone.

Step 3: The crude material from above (120 mg, 0.335 mmol) was dissolved in dichloromethane (5.0 mL) and triethylamine (58.9 mg, 0.582 mmol, 1.73 equiv) followed by n-propyl chloroformate (53.5 mg, 0.436 mmol, 1.30 equiv) were added at 0° C. After 1 h, the mixture was diluted with ethyl acetate (50 mL) and the organic layer was washed with saturated sodium bicarbonate, brine, dried over anhydrous sodium sulfate, and concentrated under reduced pressure. The crude material was purified by column chromatography (ethyl acetate/hexanes) to afford A033 as a white solid (30 mg, 20%) upon lyophilization.

¹H NMR (400 MHz, DMSO-d₆) δ 8.19 (d, J=8.6 Hz, 1H), 7.92 (d, J=1.2 Hz, 1H), 7.63 (dd, J=8.6, 1.5 Hz, 1H), 4.23-4.09 (m, 1H), 4.08-3.89 (m, 3H), 3.73-3.60 (m, 1H), 3.57-3.44 (m, 2H), 3.43-3.34 (m, 1H), 3.06 (s, 2H), 2.98 (s, 2H), 1.90 (s, 4H), 1.68-1.50 (m, 2H), 1.25 (dd, J=6.9, 2.5 Hz, 6H), 0.89 (t, J=7.4 Hz, 3H).

LCMS (ESI-TOF) m/z 444.1 [M+H⁺] with a purity of >97%.

### Propyl 4-(9-chloro-7-(2-methoxy-2-oxoethyl)-5,6,7,8-tetrahydroacridine-3-carbonyl)piperazine-1-carboxylate (A034)

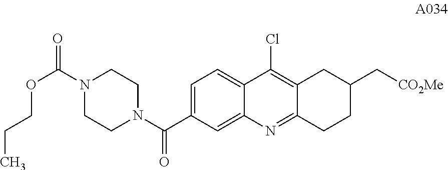 [SMILES: CCCOC(=O)N1CCN(C(=O)c2ccc3c(Cl)c4c(nc3c2)CCC(CC(=O)OC)C4)CC1]

Compound A034 was prepared according to General Procedure A, B and C2 using methyl 2-(4-oxocyclohexyl)acetate (General Procedure A) and n-propyl piperazine-1-carboxylate (General Procedure C2) as starting materials.

¹H NMR (400 MHz, DMSO-d₆) δ 8.19 (d, J=8.4 Hz, 1H), 7.95 (s, 1H), 7.65 (dd, J=8.8, 1.6 Hz 1H), 3.98 (t, J=6.4 Hz, 2H), 3.65-3.60 (m, 5H), 3.47-3.26 (m, 6H), 3.23-3.16 (m, 1H), 3.15-3.08 (m, 2H), 2.66-2.50 (m, 3H), 2.32-2.29 (m, 1H), 2.03(d, J=10.0 Hz, 1H), 1.66-1.55(m, 3H), 0.89 (t, J=7.6 Hz, 3H).

LCMS (ESI-TOF) m/z 488.3 [M+H⁺] with a purity of >95%.

### Propyl 4-(9-chloro-5,6,7,8-tetrahydroacridine-3-carbonyl)-2,2-dimethylpiperazine-1-carboxylate (A035)

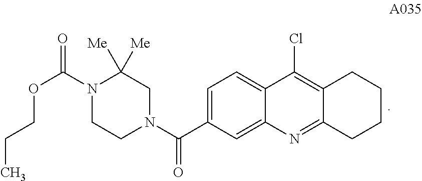 [SMILES: CCCOC(=O)N1CCN(C(=O)c2ccc3c(Cl)c4c(nc3c2)CCCC4)CC1(C)C]

Step 1: According to General Procedure C1, commercially available 9-chloro-5,6,7,8-tetrahydroacridine-3-carboxylic acid was reacted with tert-butyl 2,2-dimethylpiperazine-1-carboxylate to afford tert-butyl 4-(9-chloro-5,6,7,8-tetrahydroacridine-3-carbonyl)-2,2-dimethylpiperazine-1-carboxylate.

Step 2: The crude intermediate was dissolved in dichloromethane (5 mL) and trifluoroacetic acid (5 mL) for 4 h, then concentrated under reduced pressure to give (9-chloro-5,6,7,8-tetrahydroacridin-3-yl)(3,3-dimethylpiperazin-1-yl)methanone trifluoroacetate salt.

Step 3: The crude material from above was dissolved in N,N-dimethylformamide (10 mL) at 4° C. was added N,N-diisopropylethylamine (excess) and propyl chloroformate (excess). The resulting mixture was stirred for 2 h at 4° C. followed by 4 h at room temperature. The mixture was quenched with brine and extracted with ethyl acetate. The organic layer was dried over anhydrous sodium sulfate, filtered and concentrated under reduced pressure. The crude material was purified by preparative-HPLC to afford A035 (3%) as a yellow oil.

LCMS (ESI-TOF) m/z 444.2 [M+H⁺].

### Propyl 7-(9-chloro-5,6,7,8-tetrahydroacridine-3-carbonyl)-4,7-diazaspiro[2.5]octane-4-carboxylate (A036)

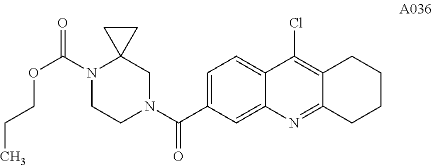 [SMILES: CCCOC(=O)N1CCN(C(=O)c2ccc3c(Cl)c4c(nc3c2)CCCC4)CC12CC2]

Step 1: According to General Procedure C1, commercially available 9-chloro-5,6,7,8-tetrahydroacridine-3-carboxylic acid was reacted with tert-butyl 4,7-diazaspiro[2.5]octane-4-carboxylate to afford tert-butyl 7-(9-chloro-5,6,7,8-tetrahydroacridine-3-carbonyl)-4,7-diazaspiro[2.5]octane-4-carboxylate.

Step 2: The crude intermediate was dissolved in dichloromethane (5 mL) and trifluoroacetic acid (5 mL) for 4 h, then concentrated under reduced pressure to give (9-chloro-5,6,7,8-tetrahydroacridin-3-yl)(4,7-diazaspiro[2.5]octan-7-yl)methanone trifluoroacetate salt.

Step 3: The crude material from above was dissolved in N,N-dimethylformamide (10 mL) at 4° C. was added N,N-diisopropylethylamine (excess) and propyl chloroformate (excess). The resulting mixture was stirred for 2 h at 4° C. followed by 4 h at room temperature. The mixture was quenched with brine and extracted with ethyl acetate. The organic layer was dried over anhydrous sodium sulfate, filtered and concentrated under reduced pressure. The crude material was purified by preparative-HPLC to afford A036 (3%) as a yellow oil.

¹H NMR (400 MHz, DMSO-d₆) δ 8.19 (d, J=8.6 Hz, 1H), 8.00 (s, 1H), 7.69 (dd, J=8.6, 1.5 Hz, 1H), 3.98 (t, J=6.4 Hz, 2H), 3.79-3.46 (m, 7H), 3.07 (s, 2H), 2.99 (s, 2H), 1.90 (s, 4H), 1.58 (dd, J=14.1, 7.1 Hz, 2H), 0.89 (t, J=7.4 Hz, 3H), 0.73 (br s, 4H).

LCMS (ESI-TOF) m/z 442.1 [M+H⁺] with a purity of >96%.

### Propyl 4-(9-chloro-5,6,7,8-tetrahydroacridine-3-carbonyl)-4,7-diazaspiro[2.5]octane-7-carboxylate (A037)

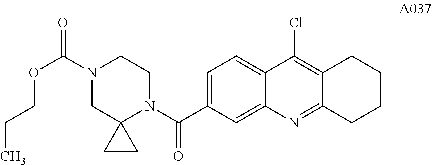 [SMILES: CCCOC(=O)N1CCN(C(=O)c2ccc3c(Cl)c4c(nc3c2)CCCC4)C2(CC2)C1]

Step 1: According to General Procedure C1, commercially available 9-chloro-5,6,7,8-tetrahydroacridine-3-carboxylic acid was reacted with tert-butyl 4,7-diazaspiro[2.5]octane-7-carboxylate to afford tert-butyl 4-(9-chloro-5,6,7,8-tetrahydroacridine-3-carbonyl)-4,7-diazaspiro[2.5]octane-7-carboxylate.

Step 2: The crude intermediate was dissolved in dichloromethane (5 mL) and trifluoroacetic acid (5 mL) for 4 h, then concentrated under reduced pressure to give (9-chloro-5,6,7,8-tetrahydroacridin-3-yl)(4,7-diazaspiro [2.5]octan-4-yl)methanone trifluoroacetate salt.

Step 3: The crude material from above was dissolved in N,N-dimethylformamide (10 mL) at 4° C. was added N,N-diisopropylethylamine (excess) and propyl chloroformate (excess). The resulting mixture was stirred for 2 h at 4° C. followed by 4 h at room temperature. The mixture was quenched with brine and extracted with ethyl acetate. The organic layer was dried over anhydrous sodium sulfate, filtered and concentrated under reduced pressure. The crude material was purified by preparative-HPLC to afford A037 (4%) as a yellow oil.

¹H NMR (400 MHz, DMSO-d₆) δ 8.20 (d, J=8.7 Hz, 1H), 7.92 (s, 1H), 7.64 (s, 1H), 3.99 (t, J=6.4 Hz, 2H), 3.83-3.15 (m, 6H), 3.07 (s, 2H), 2.99 (s, 2H), 1.90 (s, 4H), 1.64-1.53 (m, 2H), 1.07-0.76 (m, 6H), 0.58 (s, 1H).

LCMS (ESI-TOF) m/z 442.1 [M+H⁺] with a purity of >94%.

### Butyl 4-(9-chloro-5,6,7,8-tetrahydroacridine-3-carbonyl)piperazine-1-carboxylate (A038)

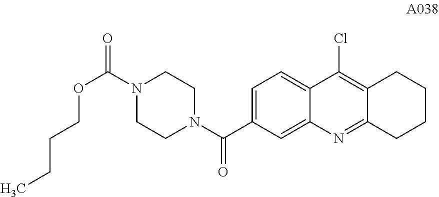 [SMILES: CCCCOC(=O)N1CCN(C(=O)c2ccc3c(Cl)c4c(nc3c2)CCCC4)CC1]

Intermediate (9-chloro-5,6,7,8-tetrahydroacridin-3-yl)(piperazin-1-yl)methanone was subjected to General Procedure C1 with butyl chloroformate to obtain A038.

¹H NMR (400 MHz, DMSO-d₆) δ 8.20 (d, J=8.5 Hz, 1H), 7.95 (d, J=1.3 Hz, 1H), 7.66 (dd, J=8.5, 1.5 Hz, 1H), 4.03 (t, J=6.5 Hz, 2H), 3.83-3.54 (m, 8H), 3.07 (s, 2H), 3.00 (s, 2H), 1.90 (s, 4H), 1.62-1.50 (m, 2H), 1.43-1.27 (m, 2H), 0.90 (t, J=7.3 Hz, 3H).

LCMS (ESI-TOF) m/z 430.2 [M+H⁺] with a purity of >96%.

### (9-Chloro-5,6,7,8-tetrahydroacridin-3-yl)(9-(5-methylisoxazole-3-carbonyl)-3,9-diazabicyclo[3.3.1]nonan-3-yl)methanone (A039)

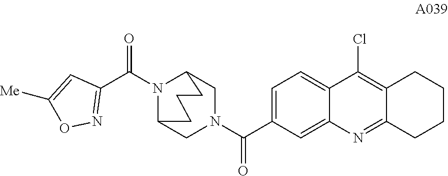 [SMILES: Cc1cc(C(=O)N2C3CCCC2CN(C(=O)c2ccc4c(Cl)c5c(nc4c2)CCCC5)C3)no1]

Intermediate (3,9-diazabicyclo[3.3.1]nonan-3-yl)(9-chloro-5,6,7,8-tetrahydroacridin-3-yl)methanone from synthesis of A013 was subjected to General Procedure C1 with 5-methylisoxazole-3-carboxylic acid to afford A039.

¹H NMR (400 MHz, DMSO-d₆) δ 8.26 (t, J=8.4 Hz, 1H), 7.93 (s, 1H), 7.68 (d, J=8.4 Hz, 1H), 6.50 (d, J=11.2 Hz, 1H), 4.86-4.46 (m, 3H), 3.80-3.48 (m, 2H), 3.23-3.14 (m, 1H), 3.09 (s, 2H), 3.00 (s, 2H), 2.46 (d, J=19.4 Hz, 3H), 2.11 (s, 1H), 2.01-1.53 (m, 9H).

LCMS (ESI-TOF) m/z 479.2 [M+H⁺] with purity >96%.

### (9-Chloro-5,6,7,8-tetrahydroacridin-3-yl)(9-(5-methyl-1H-pyrazole-3-carbonyl)-3,9-diazabicyclo[3.3.1]nonan-3-yl)methanone (A040)

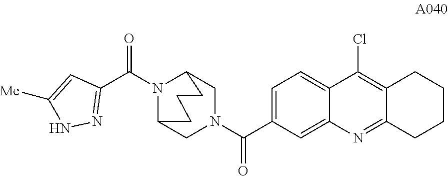 [SMILES: Cc1cc(C(=O)N2C3CCCC2CN(C(=O)c2ccc4c(Cl)c5c(nc4c2)CCCC5)C3)n[nH]1]

Intermediate (3,9-diazabicyclo[3.3.1]nonan-3-yl)(9-chloro-5,6,7,8-tetrahydroacridin-3-yl)methanone from synthesis of A013 was subjected to General Procedure C1 with 5-methyl-1H-pyrazole-3-carboxylic acid to afford A040.

¹H NMR (400 MHz, DMSO-d₆) δ 8.24 (t, J=7.8 Hz, 1H), 7.91 (s, 1H), 7.66 (d, J=8.6 Hz, 1H), 6.33 (d, J=9.2 Hz, 1H), 5.22-4.46 (m, 3H), 3.82-3.57 (m, 3H), 3.08 (s, 2H), 3.00 (s, 2H), 2.24 (d, J=16.3 Hz, 3H), 2.17-2.03 (m, 1H), 1.99-1.48 (m, 8H).

LCMS (ESI-TOF) m/z 478.2 [M+H⁺] with purity >94%.

### Butyl 3-(9-chloro-5,6,7,8-tetrahydroacridine-3-carbonyl)-3,9-diazabicyclo[3.3.1]nonane-9-carboxylate (A041)

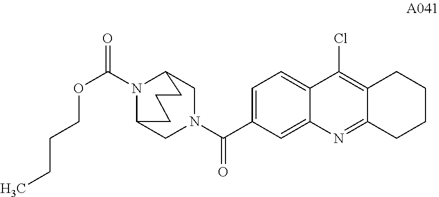 [SMILES: CCCCOC(=O)N1C2CCCC1CN(C(=O)c1ccc3c(Cl)c4c(nc3c1)CCCC4)C2]

Intermediate (3,9-diazabicyclo [3.3.1]nonan-3-yl)(9-chloro-5,6,7,8-tetrahydroacridin-3-yl)methanone from synthesis of A013 was subjected to General Procedure C1 with butyl chloroformate as reagent to afford A041.

¹H NMR (400 MHz, DMSO-d₆) δ 8.24 (d, J=8.6 Hz, 1H), 7.89 (s, 1H), 7.64 (d, J=8.6 Hz, 1H), 4.61 (d, J=13.1 Hz, 1H), 4.24 (s, 1H), 4.16-3.89 (m, 3H), 3.73-3.55 (m, 2H), 3.17-3.03 (m, 3H), 2.99 (s, 2H), 2.13-1.97 (m, 1H), 1.99-1.26 (m, 13H), 1.00-0.75 (m, 3H).

LCMS (ESI-TOF) m/z 470.3 [M+H⁺] with purity >96%.

### 2-Methyl 1-propyl 4-(9-chloro-5,6,7,8-tetrahydroacridine-3-carbonyl)piperazine-1,2-dicarboxylate (A042)

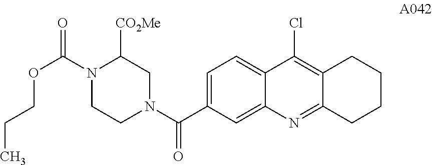 [SMILES: CCCOC(=O)N1CCN(C(=O)c2ccc3c(Cl)c4c(nc3c2)CCCC4)CC1C(=O)OC]

Step 1: According to General Procedure C1, commercially available 9-chloro-5,6,7,8-tetrahydroacridine-3-carboxylic acid was reacted with tert-butyl methyl piperazine-1,2-dicarboxylate to afford 1-(tert-butyl) 2-methyl 4-(9-chloro-5,6,7,8-tetrahydroacridine-3-carbonyl)piperazine-1,2-dicarboxylate.

Step 2: To a solution of the the above intermediate (184.2 mg, 0.378 mmol) in dichloromethane (1.1 mL) was added trifluoroacetic acid (0.59 mL, 7.71 mmol, 20 equiv). The resulting mixture was stirred for 2 h before concentrating under reduced pressure to give methyl 4-(9-chloro-5,6,7,8-tetrahydroacridine-3-carbonyl)piperazine-2-carboxylate.

Step 3: The crude material from above (104 mg, 0.268 mmol) was dissolved in dichloromethane (1.5 mL) and triethylamine (0.080 mL, 0.574 mmol, 2.15 equiv) and propyl chloroformate (0.050 mL, 0.445 mmol, 1.67 equiv) were added. The mixture was stirred for 1 h before quenching by the addition of saturated sodium bicarbonate. The aqueous layer was extracted 3 times with ethyl acetate and the combined organics were dried over anhydrous sodium sulfate, filtered and concentrated under reduced pressure. The crude material was purified by column chromatography (ethyl acetate/hexanes) to afford A042 as a yellow solid (88.7 mg, 70%) upon lyophilization.

¹H NMR (400 MHz, DMSO-d₆) δ 8.21 (d, J=8.6 Hz, 1H), 7.85 (s, 1H), 7.58 (d, J=8.5 Hz, 1H), 4.86-4.32 (m, 3H), 4.09-3.85 (m, 3H), 3.78-3.46 (m, 4H), 3.16-2.99 (m, 6H), 1.90 (s, 4H), 1.64-1.48 (m, 2H), 0.94-0.78 (m, 3H).

LCMS (ESI-TOF) m/z 474.1 [M+H⁺] with a purity of >96%.

### Propyl 4-(9-chloro-5,6,7,8-tetrahydroacridine-3-carbonyl)-3,5-dimethylpiperazine-1-carboxylate (A043)

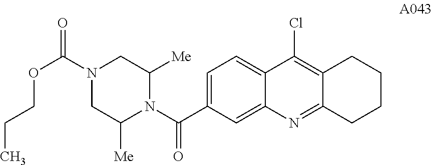 [SMILES: CCCOC(=O)N1CC(C)N(C(=O)c2ccc3c(Cl)c4c(nc3c2)CCCC4)C(C)C1]

Step 1: According to General Procedure C1, commercially available 9-chloro-5,6,7,8-tetrahydroacridine-3-carboxylic acid was reacted with tert-butyl 3,5-dimethylpiperazine-1-carboxylate to give tert-butyl 4-(9-chloro-5,6,7,8-tetrahydroacridine-3-carbonyl)-3,5-dimethylpiperazine-1-carboxylate.

Step 2: The resulting intermediate (200 mg, 0.437 mmol) was dissolved in trifluoroacetic acid (1 mL) and dichloromethane (10 mL) for 24 h. The mixture was diluted with dichloromethane (50 mL) and the organic layer was washed with saturated sodium bicarbonate, brine, dried over anhydrous sodium sulfate, filtered and concentrated under reduced pressure to give (9-chloro-5,6,7,8-tetrahydroacridin-3-yl) (2,6-dimethylpiperazin-1-yl)methanone .

Step 3: The crude material from above (120 mg, 0.335 mmol) was dissolved in dichloromethane (5.0 mL) and triethylamine (58.9 mg, 0.582 mmol, 1.73 equiv) followed by n-propyl chloroformate (53.5 mg, 0.436 mmol, 1.30 equiv) were added at 0° C. After 1 h, the mixture was diluted with ethyl acetate (50 mL) and the organic layer was washed with saturated sodium bicarbonate, brine, dried over anhydrous sodium sulfate, and concentrated under reduced pressure. The crude material was purified by column chromatography (ethyl acetate/hexanes) to afford A043 as a white solid (50 mg, 34%) upon lyophilization.

¹H NMR (400 MHz, DMSO-d₆) δ 8.19 (d, J=8.6 Hz, 1H), 7.91 (d, J=1.2 Hz, 1H), 7.62 (dd, J=8.6, 1.5 Hz, 1H), 4.73-3.62 (m, 6H), 3.23-2.93 (m, 6H), 1.90 (s, 4H), 1.58 (dq, J=13.9, 7.0 Hz, 2H), 1.20 (s, 6H), 0.89 (t, J=7.4 Hz, 3H).

LCMS (ESI-TOF) m/z 444.1 [M+H⁺] with a purity of >95%.

### Methyl 9-chloro-6-(4-(propoxycarbonyl)piperazine-1-carbonyl)-1,2,3,4-tetrahydroacridine-3-carboxylate (A044)

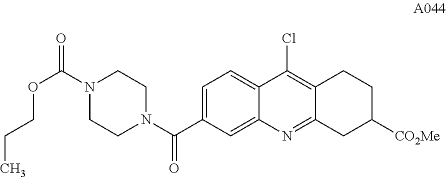 [SMILES: CCCOC(=O)N1CCN(C(=O)c2ccc3c(Cl)c4c(nc3c2)CC(C(=O)OC)CC4)CC1]

Compound A044 was prepared according to General Procedure A, B, and C2 using methyl 3-oxocyclohexanecarboxylate (General Procedure A) and n-propyl piperazine-1-carboxylate (General Procedure C2) as starting materials.

¹H NMR (400 MHz, DMSO-d₆) δ 8.20 (d, J=8.8 Hz, 1H), 7.97 (s, 1H), 7.67 (d, J=10.0 Hz, 1H), 3.8 (t, J=6.8 Hz, 2H), 3.66 (s, 5H), 3.54-3.31 (m, 6H), 3.27-3.23 (m, 2H), 3.09-3.04 (m, 3H), 2.32 (m, 1H), 2.01 (br s, 1H), 1.59-1.57 (m, 2H), 0.89 (t, J=6.8 Hz, 3H).

LCMS (ESI-TOF) m/z 474.4 [M+H⁺] with a purity of >97%.

### Propyl 4-(7-(aminomethyl)-9-chloro-5,6,7,8-tetrahydroacridine-3-carbonyl)piperazine-1-carboxylate (A045)

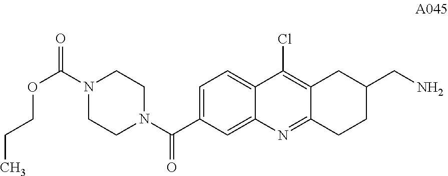 [SMILES: CCCOC(=O)N1CCN(C(=O)c2ccc3c(Cl)c4c(nc3c2)CCC(CN)C4)CC1]

Compound A045 was prepared using A046 as starting material according to General Procedure D.

¹H NMR (400 MHz, DMSO-d₆) δ 8.19 (d, J=8.8 Hz, 1H), 7.94 (s, 1H), 7.65 (dd, J=1.6 Hz, 1H), 3.98 (t, J=6.8 Hz, 2H), 3.66-3.23 (m, 8H), 3.26-3.00 (m, 4H), 2.69-2.49 (m, 2H), 2.09-2.07 (m, 1H), 1.82-1.46 (m, 6H), 0.89 (t, J=7.6 Hz, 3H).

LCMS (ESI-TOF) m/z 455.4 [M+H⁺] with a purity of >98%.

### Propyl 4-(9-chloro-7-cyano-5,6,7,8-tetrahydroacridine-3-carbonyl)piperazine-1-carboxylate (A046)

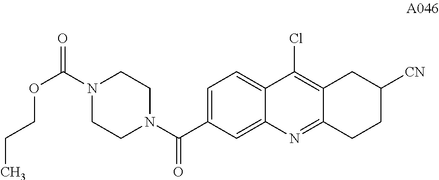 [SMILES: CCCOC(=O)N1CCN(C(=O)c2ccc3c(Cl)c4c(nc3c2)CCC(C#N)C4)CC1]

Compound A046 was prepared according to General Procedure A, B and C2 using 4-oxocyclohexanecarbonitrile (General Procedure A) and n-propyl piperazine-1-carboxylate (General Procedure C2) as starting materials.

¹H NMR (400 MHz, DMSO-d₆) δ 8.22 (d, J=8.4 Hz, 1H), 7.98 (s, 1H), 7.69 (dd, J=8.4, 1.6 Hz, 1H), 3.96 (t, J=6.4 Hz, 2H), 3.66-3.31 (m, 8H), 3.21-3.16 (m, 4H), 2.32-2.16 (m, 2H), 1.60-1.55 (m, 3H), 0.89 (t, J=7.6 Hz, 3H).

LCMS (ESI-TOF) m/z 441.2 [M+H⁺] with a purity of >98%.

### Propyl 4-(6-allyl-9-chloro-5,6,7,8-tetrahydroacridine-3-carbonyl)piperazine-1-carboxylate (A047)

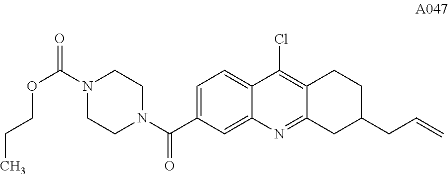 [SMILES: C=CCC1CCc2c(nc3cc(C(=O)N4CCN(C(=O)OCCC)CC4)ccc3c2Cl)C1]

Compound A047 was prepared according to General Procedure A, B, and C2 using 3-allylcyclohexanone (General Procedure A) and n-propyl piperazine-1-carboxylate (General Procedure C2) as starting materials.

¹H NMR (400 MHz, DMSO-d₆) δ 8.19 (d, J=8.8 Hz, 1H), 7.94 (s, 1H), 7.65 (dd, J=8.4, 1.6 Hz, 1H), 5.95-5.85 (m, 1H), 5.13-5.07 (t, J=14.4 Hz, 2H), 3.98 (t, J=6.4 Hz, 2H), 3.66-3.31 (m, 8H), 3.17-3.10 (m, 2H), 2.92-2.83 (m, 1H), 2.76-2.69 (m, 1H), 2.19-2.15 (m, 2H), 2.07-1.95 (m, 2H), 1.67-1.46 (m, 3H), 0.89 (t, J=7.2 Hz, 3H).

LCMS (ESI-TOF) m/z 456.2 [M+H⁺] with a purity of >97%.

### Propyl 4-(9-chloro-5,6,7,8-tetrahydroacridine-3-carbonyl)-2-(methylcarbamoyl)piperazine-1-carboxylate (A048)

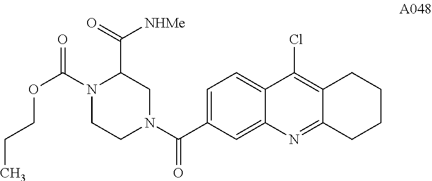 [SMILES: CCCOC(=O)N1CCN(C(=O)c2ccc3c(Cl)c4c(nc3c2)CCCC4)CC1C(=O)NC]

Step 1: To a solution of Compound A042 (300.5 mg, 0.634 mmol) in methanol (2 mL), 1,4-dioxane (1 mL) and water (2 mL) was added lithium hydroxide (34.4 mg, 1.436 mmol, 2.26 equiv). Upon completion, ethyl acetate was added and the pH was adjusted to 3 using concentrated hydrochloric acid. The organic layer was separated and the aqueous phase was extracted with ethyl acetate thrice and the combined organics were dried over anhydrous sodium sulfate, filtered and concentrated under reduced pressure to afford 4-(9-chloro-5,6,7,8-tetrahydroacridine-3-carbonyl)-1-(propoxycarbonyl)piperazine-2-carboxylic acid.

Step 2: To a solution of the crude intermediate (150 mg, 0.326 mmol) in dichloromethane (2 mL) and N,N-dimethylformamide (5 μL) was added oxalyl chloride (0.2 mL, 0.489 mmol, 1.5 equiv). After 2 h, the solvent was removed under reduced pressure. The residue (77.8 mg, 0.163 mmol) was re-dissolved in tetrahydrofuran (1 mL) and triethylamine (32.9 mg, 0.325 mmol, 2 equiv) followed by methylamine (5.05 mg, 0.163 mmol, 1 equiv) were added. After 20 min, saturated ammonium chloride was added and the mixture was extracted with ethyl acetate. The combined organics were washed with saturated sodium bicarbonate, brine, dried over anhydrous sodium sulfate, filtered and concentrated under reduced pressure. The crude material was purified by column chromatography (ethyl acetate/dichloromethane) to afford compound A048 as a white solid (2.8 mg, 4%) upon lyophilization.

LCMS (ESI-TOF) m/z 473.2 [M+H⁺] with a purity of >95%.

### 4-(9-Chloro-5,6,7,8-tetrahydroacridine-3-carbonyl)-N-propylpiperazine-1-carboxamide (A049)

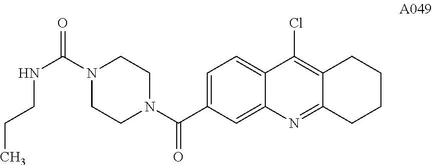 [SMILES: CCCNC(=O)N1CCN(C(=O)c2ccc3c(Cl)c4c(nc3c2)CCCC4)CC1]

Step 1: General Procedure C1 was performed between commercially available 9-chloro-5,6,7,8-tetrahydroacridine-3-carboxylic acid and tert-butyl piperazine-1-carboxylate as starting materials to obtain tert-butyl 4-(9-chloro-5,6,7,8-tetrahydroacridine-3-carbonyl)piperazine-1-carboxylate.

Step 2: The resulting intermediate (800 mg, 1.861 mmol) was dissolved in trifluoroacetic acid (2 mL) and dichloromethane (15 mL) for 24 h. The mixture was diluted with dichloromethane (50 mL) and the organic layer was washed with saturated sodium bicarbonate, brine, dried over anhydrous sodium sulfate, filtered and concentrated under reduced pressure to give (9-chloro-5,6,7,8-tetrahydroacridin-3-yl) (piperazin-1-yl)methanone.

Step 3: Propylamine (11 μL, 0.14 mmol, 1 equiv), N,N-diisoproylethylamine (0.1 mL, 0.57 mmol, 4.07 equiv) and N,N′-disuccinimidyl carbonate (56.3 mg, 0.21 mmol, 1.5 equiv) were dissolved in dichloromethane (1 mL). After 2 h, a solution of intermediate (46.2 mg, 0.14 mmol) and N,N-diisoproylethylamine (0.15 mL, 0.86 mmol, 6.15 equiv) in dichloromethane (2 mL) was added. After 1 h of stirring, the mixture was concentrated and purified by column chromatography (methanol/dichloromethane) to afford A049 as a white solid (30 mg, 52%) upon lyophilization.

¹H NMR (400 MHz, DMSO-d₆) δ 8.19 (d, J=8.6 Hz, 1H), 7.93 (d, J=1.2 Hz, 1H), 7.65 (dd, J=8.6, 1.6 Hz, 1H), 6.55 (t, J=5.4 Hz, 1H), 3.76-3.35 (m, 8H), 3.11-2.90 (m, 6H), 1.90 (s, 4H), 1.51-1.33 (m, 2H), 0.83 (t, J=7.4 Hz, 3H).

LCMS (ESI-TOF) m/z 415.2 [M+H⁺] with a purity of >99%.

### 1-(4-(9-Chloro-5,6,7,8-tetrahydroacridine-3-carbonyl)piperazin-1-yl)pent-4-en-1-one (A050)

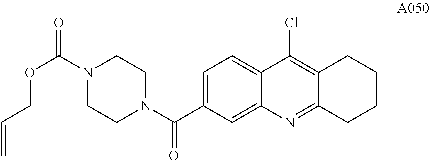 [SMILES: C=CCOC(=O)N1CCN(C(=O)c2ccc3c(Cl)c4c(nc3c2)CCCC4)CC1]

Intermediate (9-chloro-5,6,7,8-tetrahydroacridin-3-yl)(piperazin-1-yl)methanone (0.1 mmol) and N,N-diisopropylethylamine (0.175 mL, 1.0 mmol, 10 equiv) were stirred in N,N-dimethylformamide (10 mL) at 4° C. for 15 min before pent-4-enoyl chloride (0.11 mL, 1.0 mmol, 10 equiv) was added dropwise. The reaction was stirred for 2 h at the same temperature before warming to room temperature for another 4 h. The mixture was then quenched with brine and extracted with ethyl acetate. The organic layer was dried over anhydrous sodium sulfate, filtered and concentrated under reduced pressure. The crude material was purified by preparative-HPLC to affod A050 (6.55 mg, 16%) as a yellow oil.

¹H NMR (400 MHz, DMSO-d₆) δ 8.22 (d, J=8.6 Hz, 1H), 7.97 (d, J=1.2 Hz, 1H), 7.68 (dd, J=8.6, 1.5 Hz, 1H), 5.95-5.77 (m, 1H), 5.05 (d, J=17.2 Hz, 1H), 4.96 (d, J=9.7 Hz, 1H), 3.83-3.26 (m, 8H), 3.08 (s, 2H), 2.99 (s, 2H), 2.43 (s, 2H), 2.25 (dd, J=13.9, 6.6 Hz, 2H), 1.91 (d, J=2.8 Hz, 4H).

LCMS (ESI-TOF) m/z 412.2 [M+H⁺] with purity >94%.

### 3-Methyl 1-propyl 4-(9-chloro-5,6,7,8-tetrahydroacridine-3-carbonyl)piperazine-1,3-dicarboxylate (A051)

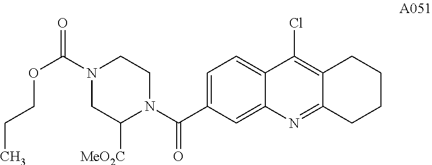 [SMILES: CCCOC(=O)N1CCN(C(=O)c2ccc3c(Cl)c4c(nc3c2)CCCC4)C(C(=O)OC)C1]

Compound A051 was performed according to General Procedure C1 between commercially available 9-chloro-5,6,7,8-tetrahydroacridine-3-carboxylic acid and 3-methyl-1-propylpiperazine-1,3-dicarboxylate as starting materials.

¹H NMR (400 MHz, 80° C., DMSO-d₆) δ 8.21 (d, J=8.5 Hz, 1H), 7.90 (s, 1H), 7.60 (d, J=8.6 Hz, 1H), 5.24-4.73 (m, 2H), 4.41 (d, J=14.1 Hz, 1H), 3.97 (t, J=6.3 Hz, 2H), 3.90-3.68 (m, 5H), 3.38-3.20 (m, 3H), 3.00 (br s, 3H), 1.91 (s, 4H), 1.58 (dq, J=14.3, 7.0 Hz, 2H), 0.89 (t , J=7.3 Hz, 3H).

LCMS (ESI-TOF) m/z 474.1 [M+H⁺] with a purity of >97%.

### Propyl 2-(acetoxymethyl)-4-(9-chloro-5,6,7,8-tetrahydroacridine-3-carbonyl)piperazine-1-carboxylate (A052)

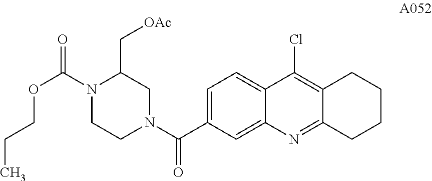 [SMILES: CCCOC(=O)N1CCN(C(=O)c2ccc3c(Cl)c4c(nc3c2)CCCC4)CC1COC(C)=O]

Step 1: According to General Procedure C1, commercially available 9-chloro-5,6,7,8-tetrahydroacridine-3-carboxylic acid was reacted with tert-butyl 2-(hydroxymethyl)piperazine-1-carboxylate to give tert-butyl 4-(9-chloro-5,6,7,8-tetrahydroacridine-3-carbonyl)-2-(hydroxymethyl)piperazine-1-carboxylate.

Step 2: Intermediate from Step 1 (92 mg, 0.20 mmol) and 4-dimethylaminopyridine (1.22 mg, 0.010 mmol, 0.05 equiv) was dissolved in dichloromethane (1 mL) at 0° C. Triethylamine (83 μL, 0.60 mmol, 3 equiv) followed by acetic anhydride (22 μL, 0.24 mmol, 1.2 equiv) was added. The reaction mixture was stirred for 15 min before diluting with dichloromethane (50 mL). The organic layer was separated and washed with saturated sodium bicarbonate, brine, dried over anhydrous sodium sulfate, filtered and concentrated to give tert-butyl 2-(acetoxymethyl)-4-(9-chloro-5,6,7,8-tetrahydroacridine-3-carbonyl)piperazine-1-carboxylate.

Step 3: The resulting crude intermediate (80 mg, 0.159 mmol) was dissolved in trifluoroacetic acid (0.4 mL) and dichloromethane (5 mL) for 24 h. The mixture was diluted with dichloromethane (50 mL) and the organic layer was washed with saturated sodium bicarbonate, brine, dried over anhydrous sodium sulfate, filtered and concentrated under reduced pressure to give (4-(9-chloro-5,6,7,8-tetrahydroacridine-3-carbonyl)piperazin-2-yl)methyl acetate.

Step 4: The crude material from above (70 mg, 0.174 mmol) was dissolved in dichloromethane (5.0 mL) and triethylamine (58.9 mg, 0.582 mmol, 3.34 equiv) followed by n-propyl chloroformate (53.5 mg, 0.436 mmol, 2.5 equiv) were added at 0° C. After 1 h, the mixture was diluted with ethyl acetate (50 mL) and the organic layer was washed with saturated sodium bicarbonate, brine, dried over anhydrous sodium sulfate, and concentrated under reduced pressure. The crude material was purified by column chromatography (ethyl acetate/hexanes) to afford A052 as a white solid (10 mg, 12%) upon lyophilization.

¹H NMR (400 MHz, DMSO-d₆) δ 8.20 (d, J=8.6 Hz, 1H), 7.93 (s, 1H), 7.63 (s, 1H), 4.59-3.34 (m, 9H), 3.24-2.74 (m, 6H), 2.10-1.66 (m, 7H), 1.67-1.48 (m, 2H), 0.89 (t, J=7.3 Hz, 3H).

LCMS (ESI-TOF) m/z 488.1 [M+H⁺] with a purity of >97%.

### Propyl 4-(9-chloro-6-(pyridin-2-yl)-5,6,7,8-tetrahydroacridine-3-carbonyl)piperazine-1-carboxylate (A053)

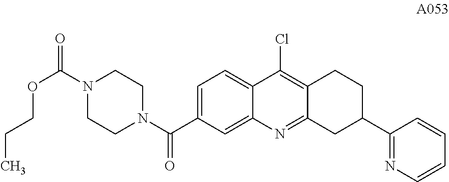 [SMILES: CCCOC(=O)N1CCN(C(=O)c2ccc3c(Cl)c4c(nc3c2)CC(c2ccccn2)CC4)CC1]

Compound A053 was prepared according to General Procedure E, F, C2 and G using 3-(pyridin-2-yl)cyclohexanone (General Procedure E) and n-propyl piperazine-1-carboxylate (General Procedure C2) as starting materials.

¹H NMR (400 MHz, DMSO-d₆) δ 8.54 (d, J=4.4 Hz, 1H), 8.22 (d, J=8.4 Hz, 1H), 7.98 (d, J=1.2 Hz, 1H), 7.78 (dt, J=7.6, 1.6 Hz, 1H), 7.68 (dd, J=8.4, 1.6 Hz, 1H), 7.43 (d, J=8.0 Hz, 1H), 7.28-7.25 (m, 1H), 3.98 (t, J=6.4 Hz, 2H), 3.66-3.38 (m, 11H), 3.25-3.05 (m, 2H), 2.32.-2.27 (m, 1H), 2.15 (br s, 1H), 1.61-1.55 (m, 2H), 0.89 (t, J=7.2 Hz, 3H).

LCMS (ESI-TOF) m/z 493.5 [M+H⁺] with a purity of >95%.

### Propyl 4-(9-chloro-5,6,7,8-tetrahydroacridine-3-carbonyl)-2-((isobutyryloxy)methyl)piperazine-1-carboxylate (A054)

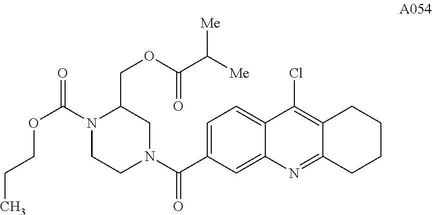 [SMILES: CCCOC(=O)N1CCN(C(=O)c2ccc3c(Cl)c4c(nc3c2)CCCC4)CC1COC(=O)C(C)C]

Step 1: According to General Procedure C1, commercially available 9-chloro-5,6,7,8-tetrahydroacridine-3-carboxylic acid was reacted with tert-butyl 2-(hydroxymethyl)piperazine-1-carboxylate to give tert-butyl 4-(9-chloro-5,6,7,8-tetrahydroacridine-3-carbonyl)-2-(hydroxymethyl)piperazine-1-carboxylate.

Step 2: Intermediate from Step 1 (92 mg, 0.20 mmol) and 4-dimethylaminopyridine (1.22 mg, 0.010 mmol, 0.05 equiv) was dissolved in dichloromethane (1 mL) at 0° C. Triethylamine (83 μL, 0.60 mmol, 3 equiv) followed by isobutyryl chloride (25.6 mg, 0.24 mmol, 1.2 equiv) was added. The reaction mixture was stirred for 15 min before diluting with dichloromethane (50 mL). The organic layer was separated and washed with saturated sodium bicarbonate, brine, dried over anhydrous sodium sulfate, filtered and concentrated to give tert-butyl 4-(9-chloro-5,6,7,8-tetrahydroacridine-3-carbonyl)-2-((isobutyryloxy)methyl)piperazine-1-carboxylate.

Step 3: The resulting crude intermediate (80 mg, 0.151 mmol) was dissolved in trifluoroacetic acid (0.5 mL) and dichloromethane (5 mL) for 24 h. The mixture was diluted with dichloromethane (50 mL) and the organic layer was washed with saturated sodium bicarbonate, brine, dried over anhydrous sodium sulfate, filtered and concentrated under reduced pressure to give (4-(9-chloro-5,6,7,8-tetrahydroacridine-3-carbonyl)piperazin-2-yl)methyl isobutyrate.

Step 4: The crude material from above (60 mg, 0.14 mmol) was dissolved in dichloromethane (5.0 mL) and triethylamine (58.9 mg, 0.582 mmol, 4.16 equiv) followed by n-propyl chloroformate (53.5 mg, 0.436 mmol, 3.12 equiv) were added at 0° C. After 1 h, the mixture was diluted with ethyl acetate (50 mL) and the organic layer was washed with saturated sodium bicarbonate, brine, dried over anhydrous sodium sulfate, and concentrated under reduced pressure. The crude material was purified by column chromatography (ethyl acetate/hexanes) to afford A054 as a white solid (15 mg, 21%) upon lyophilization.

¹H NMR (400 MHz, DMSO-d₆) δ 8.20 (d, J=8.6 Hz, 1H), 7.94 (s, 1H), 7.63 (s, 1H), 4.60-3.35 (m, 9H), 3.24-2.79 (m, 7H), 1.90 (s, 4H), 1.69-1.48 (m, 2H), 1.08 (s, 3H), 0.96-0.70 (m, 6H).

LCMS (ESI-TOF) m/z 517.2 [M+H⁺] with a purity of >96%.

### Propyl 4-(9-chloro-5,6,7,8-tetrahydroacridine-3-carbonyl)-2-(((3-methoxypropanoyl)oxy)methyl)-piperazine-1-carboxylate (A055)

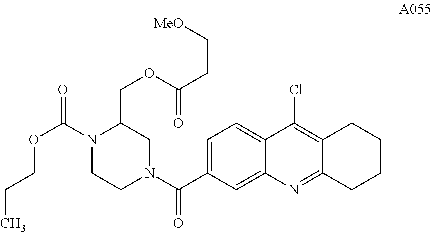 [SMILES: CCCOC(=O)N1CCN(C(=O)c2ccc3c(Cl)c4c(nc3c2)CCCC4)CC1COC(=O)CCOC]

Step 1: According to General Procedure C1, commercially available 9-chloro-5,6,7,8-tetrahydroacridine-3-carboxylic acid was reacted with tert-butyl 2-(hydroxymethyl)piperazine-1-carboxylate to give tert-butyl 4-(9-chloro-5,6,7,8-tetrahydroacridine-3-carbonyl)-2-(hydroxymethyl)piperazine-1-carboxylate.

Step 2: Intermediate from Step 1 (92 mg, 0.20 mmol) and 4-dimethylaminopyridine (4.9 mg, 0.040 mmol, 0.2 equiv) was dissolved in dichloromethane (5 mL) at 0° C. Triethylamine (42 μL, 0.30 mmol, 1.5 equiv) followed by 3-methoxypropionyl chloride (29.4 mg, 0.24 mmol, 1.2 equiv) was added. The reaction mixture was stirred for 15 min before diluting with dichloromethane (50 mL). The organic layer was separated and washed with saturated sodium bicarbonate, brine, dried over anhydrous sodium sulfate, filtered and concentrated to afford tert-butyl 4-(9-chloro-5,6,7,8-tetrahydroacridine-3-carbonyl)-2-(((3-methoxypropanoyl)oxy)methyl)piperazine-1-carboxylate.

Step 3: The resulting crude intermediate (80 mg, 0.147 mmol) was dissolved in trifluoroacetic acid (0.5 mL) and dichloromethane (5 mL) for 24 h. The mixture was diluted with dichloromethane (50 mL) and the organic layer was washed with saturated sodium bicarbonate, brine, dried over anhydrous sodium sulfate, filtered and concentrated under reduced pressure to afford (4-(9-chloro-5,6,7,8-tetrahydroacridine-3-carbonyl)piperazin-2-yl)methyl 3-methoxypropanoate.

Step 4: The crude material from above (50 mg, 0.112 mmol) was dissolved in dichloromethane (5.0 mL) and triethylamine (58.9 mg, 0.582 mmol, 5.19 equiv) followed by n-propyl chloroformate (53.5 mg, 0.436 mmol, 3.89 equiv) were added at 0° C. After 1 h, the mixture was diluted with ethyl acetate (50 mL) and the organic layer was washed with saturated sodium bicarbonate, brine, dried over anhydrous sodium sulfate, and concentrated under reduced pressure. The crude material was purified by column chromatography (ethyl acetate/hexanes) to afford A055 as a white solid (8 mg, 13%) upon lyophilization.

¹H NMR (400 MHz, DMSO-d₆) δ 8.20 (d, J=8.6 Hz, 1H), 7.92 (s, 1H), 7.63 (s, 1H), 4.58-3.46 (m, 12H), 3.23-2.85 (m, 10H), 1.90 (s, 4H), 1.66-1.50 (m, 2H), 0.89 (t, J=7.3 Hz, 3H).

LCMS (ESI-TOF) m/z 533.1 [M+H⁺] with a purity of >94%.

### Propyl 2-carbamoyl-4-(9-chloro-5,6,7,8-tetrahydroacridine-3-carbonyl)piperazine-1-carboxylate (A056)

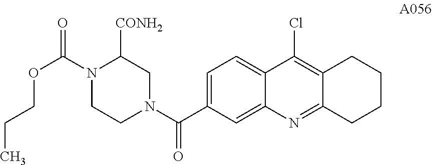 [SMILES: CCCOC(=O)N1CCN(C(=O)c2ccc3c(Cl)c4c(nc3c2)CCCC4)CC1C(N)=O]

Step 1: According to General Procedure C1, commercially available 9-chloro-5,6,7,8-tetrahydroacridine-3-carboxylic acid was reacted with tert-butyl 2-carbamoylpiperazine-1-carboxylate to give tert-butyl 2-c arbamoyl-4-(9-chloro-5,6,7,8-tetrahydroacridine-3-carbonyl)piperazine-1-carboxylate.

Step 2: The resulting crude intermediate (98.1 mg, 0.207 mmol) was dissolved in trifluoroacetic acid (0.16 mL) and dichloromethane (0.16 mL) for 20 min. The mixture was concentrated under reduced pressure and then diluted with ethyl acetate. The organic layer was washed with saturated sodium bicarbonate, dried over anhydrous sodium sulfate, filtered and concentrated under reduced pressure to give 4-(9-chloro-5,6,7,8-tetrahydroacridine-3-carbonyl)piperazine-2-carboxamide.

Step 3: The crude material from above (57.8 mg, 0.155 mmol) was dissolved in dichloromethane (1.6 mL) and triethylamine (29 mg, 0.287 mmol, 1.84 equiv) followed by n-propyl chloroformate (24.0 mg, 0.196 mmol, 1.26 equiv) were added at room temperature. The mixture was stirred for 20 min before quenching by the addition of saturated sodium bicarbonate. The aqueous layer was extracted 3 times with ethyl acetate and the combined organics were dried over anhydrous sodium sulfate, filtered and concentrated under reduced pressure. The crude material was purified by column chromatography (100% ethyl acetate) followed by preparative HPLC to afford A056 as a white solid (24.6 mg, 35%) upon lyophilization.

¹H NMR (400 MHz, 80° C., DMSO-d₆) δ 8.17 (d, J=8.6 Hz, 1H), 7.87 (s, 1H), 7.56 (d, J=8.5 Hz, 1H), 7.24-6.76 (m, 2H), 4.49-4.24 (m, 2H), 3.98 (dd, J=10.3, 6.3 Hz, 3H), 3.80 (d, J=12.8 Hz, 1H), 3.49 (dd, J=26.9, 12.2 Hz, 2H), 3.13 (dd, J=22.2, 11.8 Hz, 1H), 3.04 (s, 2H), 2.99 (s, 2H), 1.91 (s, 4H), 1.58 (dq, J=13.8, 6.8 Hz, 2H), 0.89 (t, J=7.3 Hz, 3H).

LCMS (ESI-TOF) m/z 459.1 [M+H⁺] with a purity of >97%.

### Propyl 4-(9-chloro-6-(pyrimidin-5-yl)-5,6,7,8-tetrahydroacridine-3-carbonyl)piperazine-1-carboxylate (A057)

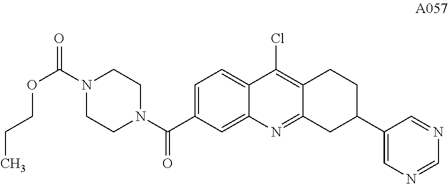 [SMILES: CCCOC(=O)N1CCN(C(=O)c2ccc3c(Cl)c4c(nc3c2)CC(c2cncnc2)CC4)CC1]

Compound A057 was prepared according to General Procedure A, B and C2 using 3-(pyrimidin-5-yl)cyclohexanone (General Procedure A) and n-propyl piperazine-1-carboxylate (General Procedure C2) as starting materials.

¹H NMR (400 MHz, DMSO-d₆) δ 9.11 (s, 1H), 8.87 (s, 2H), 8.24 (d, J=8.8 Hz, 1H), 7.98 (d, J=1.6 Hz, 1H), 7.69 (dd, J=8.8, 1.6 Hz, 1H), 3.98 (t, J=6.4 Hz, 2H), 3.41-3.33 (m, 10H), 3.26-3.24 (m, 2H), 3.10-3.06 (m, 1H), 2.33-2.32 (m, 1H), 2.21-2.10 (m, 1H), 1.61-1.58 (m, 2H), 0.89 (t, J=7.2 Hz, 3H).

LCMS (ESI-TOF) m/z 494.2 [M+H⁺] with a purity of >98%.

### Propyl 4-(9-chloro-6-(pyridin-4-yl)-5,6,7,8-tetrahydroacridine-3-carbonyl)piperazine-1-carboxylate (A058)

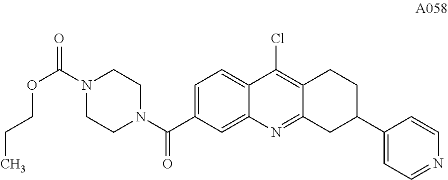 [SMILES: CCCOC(=O)N1CCN(C(=O)c2ccc3c(Cl)c4c(nc3c2)CC(c2ccncc2)CC4)CC1]

Compound A058 was prepared according to General Procedure A, B and C2 using 3-(pyridin-4-yl)cyclohexanone (General Procedure A) and n-propyl piperazine-1-carboxylate (General Procedure C2) as starting materials.

¹H NMR (400 MHz, DMSO-d₆) δ 8.54 (d, J=5.6 Hz, 2H), 8.23 (d, J=8.8 Hz, 1H), 7.98 (s, 1H), 7.68 (d, J=1.6 Hz, 1H), 7.41 (d, J=5.6 Hz, 2H), 3.98 (t, J=6.4 Hz, 2H), 3.67-3.35 (m, 8H), 3.24-3.20 (m, 4H), 3.10-3.01(m,1H), 2.23 (br s, 1H), 2.07-2.04 (m, 1H),1.60-1.55 (m, 2H), 0.89 (t, J=7.2 Hz, 3H).

LCMS (ESI-TOF) m/z 493.2 [M+H⁺] with a purity of >97%.

### Propyl 3-carbamoyl-4-(9-chloro-5,6,7,8-tetrahydroacridine-3-carbonyl)piperazine-1-carboxylate (A059)

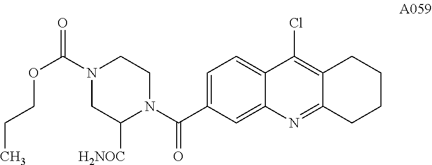 [SMILES: CCCOC(=O)N1CCN(C(=O)c2ccc3c(Cl)c4c(nc3c2)CCCC4)C(C(N)=O)C1]

Compound A059 was prepared according to General Procedure C 1 , using commercially available 9-chloro-5,6,7,8-tetrahydroacridine-3-carboxylic acid and n-propyl 3-carbamoylpiperazine-1-carboxylate as starting materials.

¹H NMR (400 MHz, 80° C., DMSO-d₆) δ 8.18 (d, J=8.5 Hz, 1H), 7.94 (s, 1H), 7.62 (d, J=8.5 Hz, 1H), 7.45-6.83 (m, 2H), 5.13-4.22 (m, 2H), 4.05-3.90 (m, 2H), 3.81 (br s, 1H), 3.47 (br s, 1H), 3.30 (d, J=9.8 Hz, 1H), 3.11-2.91 (m, 5H), 2.52 (s, 1H), 1.91 (s, 4H), 1.64-1.50 (m, 2H), 0.89 (t, J=7.4 Hz, 3H).

LCMS (ESI-TOF) m/z 459.1 [M+H⁺] with a purity of >98%.

### Propyl 4-(9-chloro-5,6,7,8-tetrahydroacridine-3-carbonyl)-2-(2-hydroxyethyl)piperazine-1-carboxylate (A060)

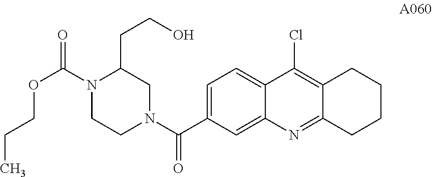 [SMILES: CCCOC(=O)N1CCN(C(=O)c2ccc3c(Cl)c4c(nc3c2)CCCC4)CC1CCO]

Step 1: According to General Procedure C1, commercially available 9-chloro-5,6,7,8-tetrahydroacridine-3-carboxylic acid was reacted with tert-butyl 2-(2-hydroxyethyl)piperazine-1-carboxylate to give tert-butyl 4-(9-chloro-5,6,7,8-tetrahydroacridine-3-carbonyl)-2-(2-hydroxyethyl)piperazine-1-carboxylate.

Step 2: The resulting crude intermediate (54.3 mg, 0.115 mmol) was dissolved in trifluoroacetic acid (1.05 mL) and dichloromethane (1.5 mL) for 30 min. The mixture was concentrated under reduced pressure and then diluted with ethyl acetate. The organic layer was washed with saturated sodium bicarbonate, dried over anhydrous sodium sulfate, filtered and concentrated under reduced pressure to give (9-chloro-5,6,7,8-tetrahydroacridin-3-yl)(3-(2-hydroxyethyl)piperazin-1-yl)methanone.

Step 3: The crude material from above (16.5 mg, 0.044 mmol) was dissolved in dichloromethane (1 mL) and triethylamine (10.88 mg, 0.108 mmol, 2.44 equiv) followed by n-propyl chloroformate (8.1 mg, 0.066 mmol, 1.5 equiv) were added at room temperature. After 20 min, saturated ammonium chloride was added and the aqueous layer was extracted with dichloromethane. The organic layers were washed with brine, dried over anhydrous sodium sulfate, and concentrated under reduced pressure. The crude material was purified by column chromatography (ethyl acetate/hexanes) to afford A060 as a white solid (4.88 mg, 24%) upon lyophilization.

¹H NMR (400 MHz, DMSO-d₆) δ 8.20 (d, J=8.6 Hz, 1H), 7.92 (s, 1H), 7.64 (d, J=6.2 Hz, 1H), 4.70-3.69 (m, 6H), 3.17-2.84 (m, 9H), 1.90 (s, 4H), 1.83-1.37 (m, 5H), 0.89 (t, J=7.3 Hz, 3H).

LCMS (ESI-TOF) m/z 460.1 [M+H⁺] with a purity of >99%.

### Propyl 2-((butyryloxy)methyl)-4-(9-chloro-5,6,7,8-tetrahydroacridine-3-carbonyl)piperazine-1-carboxylate (A061)

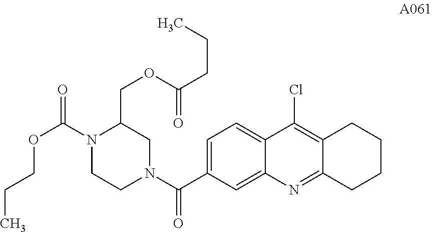 [SMILES: CCCOC(=O)N1CCN(C(=O)c2ccc3c(Cl)c4c(nc3c2)CCCC4)CC1COC(=O)CCC]

Step 1: According to General Procedure C1, commercially available 9-chloro-5,6,7,8-tetrahydroacridine-3-carboxylic acid was reacted with tert-butyl 2-(hydroxymethyl)piperazine-1-carboxylate to give tert-butyl 4-(9-chloro-5,6,7,8-tetrahydroacridine-3-carbonyl)-2-(hydroxymethyl)piperazine-1-carboxylate.

Step 2: Intermediate from Step 1 (92 mg, 0.20 mmol) and 4-dimethylaminopyridine (4.9 mg, 0.040 mmol, 0.2 equiv) was dissolved in dichloromethane (5 mL) at 0° C. Triethylamine (30.4 μL, 0.30 mmol, 1.5 equiv) followed by butyryl chloride (25.6 mg, 0.24 mmol, 1.2 equiv) was added. The reaction mixture was stirred for 15 min before diluting with dichloromethane (50 mL). The organic layer was separated and washed with saturated sodium bicarbonate, brine, dried over anhydrous sodium sulfate, filtered and concentrated to afford tert-butyl 2-((butyryloxy)methyl)-4-(9-chloro-5 ,6,7,8-tetrahydroacridine-3-carbonyl)piperazine-1-carboxylate.

Step 3: The resulting crude intermediate (80 mg, 0.151 mmol) was dissolved in trifluoroacetic acid (0.5 mL) and dichloromethane (5 mL) for 24 h. The mixture was diluted with dichloromethane (50 mL) and the organic layer was washed with saturated sodium bicarbonate, brine, dried over anhydrous sodium sulfate, filtered and concentrated under reduced pressure to give (4-(9-chloro-5,6,7,8-tetrahydroacridine-3-carbonyl)piperazin-2-yl)methyl butyrate.

Step 4: The crude material from above (50 mg, 0.116 mmol) was dissolved in dichloromethane (5.0 mL) and triethylamine (58.9 mg, 0.582 mmol, 5 equiv) followed by n-propyl chloroformate (53.5 mg, 0.436 mmol, 3.75 equiv) were added at 0° C. After 1 h, the mixture was diluted with ethyl acetate (50 mL) and the organic layer was washed with saturated sodium bicarbonate, brine, dried over anhydrous sodium sulfate, and concentrated under reduced pressure. The crude material was purified by column chromatography (ethyl acetate/hexanes) to afford A061 as a white solid (8 mg, 13%) upon lyophilization.

¹H NMR (400 MHz, DMSO-d₆) δ 8.20 (d, J=8.6 Hz, 1H), 7.93 (s, 1H), 7.64 (s, 1H), 4.65-3.36 (m, 9H), 3.24-2.91 (m, 7H), 1.90 (s, 5H), 1.58 (dq, J=14.2, 7.1 Hz, 3H), 0.97-0.54 (m, 7H).

LCMS (ESI-TOF) m/z 516.2 [M+H⁺] with a purity of >99%.

### Propyl (2S)-4-(9-chloro-7-(2-methoxy-2-oxoethyl)-5,6,7,8-tetrahydroacridine-3-carbonyl)-2-methylpiperazine-1-carboxylate (A062)

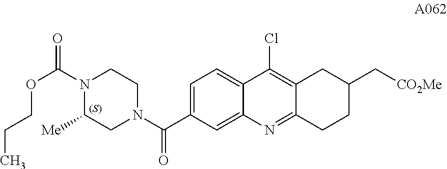

From the intermediate of General Procedure B in the synthesis of A034, General Procedure C1 was conducted with (S)-n-propyl 2-methylpiperazine-1-carboxylate to afford compound A062.

¹H NMR (400 MHz, DMSO-d₆) δ 8.21 (d, J=8.6 Hz, 1H), 7.94 (s, 1H), 7.67 (s, 1H), 4.68-3.47 (m, 9H), 3.26-2.86 (m, 6H), 2.72-2.55 (m, 3H), 2.36-2.26 (m, 1H), 2.12-1.97 (m, 1H), 1.72-1.45 (m, 3H), 1.27-0.95 (m, 3H), 0.89 (t, J=7.4 Hz, 3H).

LCMS (ESI-TOF) m/z 502.2 [M+H⁺] with a purity of >99%.

### Propyl (2R)-4-(9-chloro-7-(2-methoxy-2-oxoethyl)-5,6,7,8-tetrahydroacridine-3-carbonyl)-2-methylpiperazine-1-carboxylate (A063)

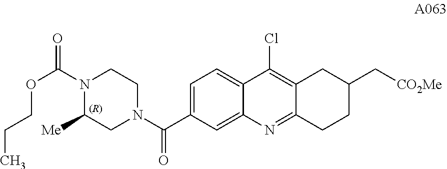

From the intermediate of General Procedure B in the synthesis of A034, General Procedure C1 was conducted with (R)-n-propyl 2-methylpiperazine-1-carboxylate to afford compound A063.

¹H NMR (400 MHz, 80° C., DMSO-d₆) δ 8.18 (d, J=8.5 Hz, 1H), 7.90 (s, 1H), 7.61 (d, J=8.6 Hz, 1H), 4.47 (s, 1H), 4.15-3.71 (m, 5H), 3.48 (s, 3H), 3.30 (s, 1H), 3.21-3.11 (m, 2H), 3.05 (s, 2H), 3.00 (s, 2H), 2.74-2.51 (m, 2H), 1.91 (s, 4H), 1.64-1.48 (m, 2H), 0.89 (t, J=7.4 Hz, 3H).

LCMS (ESI-TOF) m/z 488.1 [M+H⁺] with a purity of >99%.

### Propyl 4-(9-chloro-5,6,7,8-tetrahydroacridine-3-carbonyl)-3-(trifluoromethyl)piperazine-1-carboxylate (A064)

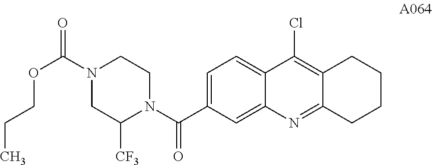 [SMILES: CCCOC(=O)N1CCN(C(=O)c2ccc3c(Cl)c4c(nc3c2)CCCC4)C(C(F)(F)F)C1]

Step 1: According to General Procedure C1, commercially available 9-chloro-5,6,7,8-tetrahydroacridine-3-carboxylic acid was reacted with tert-butyl 3-trifluoromethylpiperazine-1-carboxylate to afford tert-butyl 4-(9-chloro-5,6,7,8-tetrahydroacridine-3-carbonyl)-3-(trifluoromethyl)piperazine-1-carboxylate.

Step 2: The resulting intermediate (20 mg, 0.40 mmol) was dissolved in trifluoroacetic acid (0.2 mL) and dichloromethane (2 mL) for 24 h. The mixture was diluted with dichloromethane (50 mL) and the organic layer was washed with saturated sodium bicarbonate, brine, dried over anhydrous sodium sulfate, filtered and concentrated under reduced pressure to afford (9-chloro-5,6,7,8-tetrahydroacridin-3-yl) (2-(trifluoromethyl)piperazin-1-yl)methanone.

Step 3: The crude material from above (12 mg, 0.030 mmol) was dissolved in dichloromethane (2.0 mL) and triethylamine (58.9 mg, 0.582 mmol, 19.3 equiv) followed by n-propyl chloroformate (53.5 mg, 0.436 mmol, 14.5 equiv) were added at 0° C. After 1 h, the mixture was diluted with ethyl acetate (30 mL) and the organic layer was washed with saturated sodium bicarbonate, brine, dried over anhydrous sodium sulfate, and concentrated under reduced pressure. The crude material was purified by column chromatography (ethyl acetate/hexanes) to afford A064 as a white solid (5 mg, 34%) upon lyophilization.

¹H NMR (400 MHz, DMSO-d₆) δ 8.23 (d, J=8.6 Hz, 1H), 7.97 (s, 1H), 7.65 (d, J=8.5 Hz, 1H), 5.42 (s, 1H), 4.55-3.32 (m, 7H), 3.19-3.00 (m, 5H), 1.90 (s, 4H), 1.57 (d, J=6.4 Hz, 2H), 0.88 (s, 3H). ¹⁹F NMR (376 MHz, DMSO-d₆) δ-68.79-−69.83 (m, 3F).

LCMS (ESI-TOF) m/z 484.1 [M+H⁺] with a purity of >98%.

### Propyl 4-(9-chloro-6-(cyanomethyl)-5,6,7,8-tetrahydroacridine-3-carbonyl)piperazine-1-carboxylate (A065)

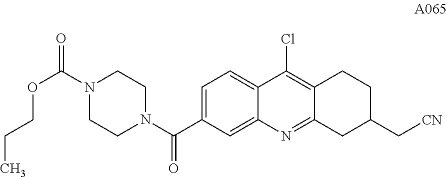 [SMILES: CCCOC(=O)N1CCN(C(=O)c2ccc3c(Cl)c4c(nc3c2)CC(CC#N)CC4)CC1]

Compound A065 was prepared according to General Procedure A, B and C2 using 2-(3-oxocyclohexyl)acetonitrile (General Procedure A) and n-propyl piperazine-1-carboxylate (General Procedure C2) as starting materials.

¹H NMR (400 MHz, DMSO-d₆) δ 8.20 (d, J=8.0 Hz, 1H), 7.96 (d, J=1.6 Hz, 1H), 7.67 (dd, J=8.4, 1.6 Hz, 1H), 3.98 (t, J=6.4 Hz, 2H), 3.66-3.26 (m, 8H), 3.25-3.15 (m, 2H), 2.98-2.84 (m, 2H), 2.82-2.71 (m, 2H), 2.32-2.28 (m, 1H), 2.16-2.12 (m, 1H), 1.67-1.55 (m, 3H), 0.89 (t, J=7.6 Hz, 3H).

LCMS (ESI-TOF) m/z 455.4 [M+H⁺] with a purity of >96%.

### Propyl 4-(7-(2-aminoethyl)-9-chloro-5,6,7,8-tetrahydroacridine-3-carbonyl)piperazine-1-carboxylate (A066)

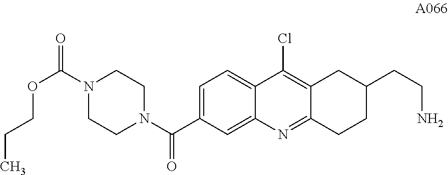 [SMILES: CCCOC(=O)N1CCN(C(=O)c2ccc3c(Cl)c4c(nc3c2)CCC(CCN)C4)CC1]

Compound A066 was prepared using A078 as starting material according to General Procedure D.

¹H NMR (400 MHz, DMSO-d₆) δ 8.19 (d, J=8.8 Hz, 1H), 7.94 (s, 1H), 7.65 (dd, J=8.4, 1.2 Hz, 1H), 6.72 (br s, 2H), 3.98 (t, J=6.4 Hz, 2H), 3.70-3.20 (m, 8H), 3.20-3.00 (m, 3H), 2.71-2.67 (m, 1H), 2.53-2.49 (m, 1H), 2.04-2.01 (m, 2H), 1.60-1.51 (m, 5H), 0.89 (t, J=7.2 Hz, 3H).

LCMS (ESI-TOF) m/z 459.2 [M+H⁺] with a purity of >95%.

### Propyl (3S)-4-(6-(aminomethyl)-9-chloro-5,6,7,8-tetrahydroacridine-3-carbonyl)-3-methylpiperazine-1-carboxylate (A067)

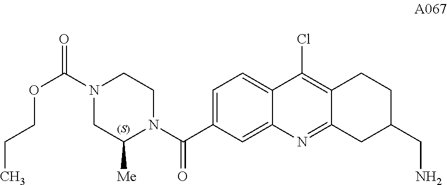

Compound A067 was prepared was prepared according to General Procedure A, B, C2 and D using 2-(3-oxocyclohexyl)acetamide (General Procedure A) and (S)-propyl 3-methylpiperazine-1-carboxylate (General Procedure C2) as starting materials.

¹H NMR (400 MHz, DMSO-d₆) δ 8.19 (d, J=8.8 Hz, 1H), 7.91 (s, 1H), 7.63 (dd, J=8.8, 1.2 Hz, 1H), 4.01-3.78 (m, 5H), 3.20-2.84 (m, 7H), 2.76-2.61 (m, 3H), 2.10-2.07 (m, 1H), 1.84 (br s, 1H), 1.60-1.48 (m, 3H), 1.17 (m, 3H), 0.89 (t, J=7.2 Hz, 3H).

LCMS (ESI-TOF) m/z 459.3 [M+H⁺] with a purity of >97%.

### Propyl 4-(9-chloro-7-((dimethylamino)methyl)-5,6,7,8-tetrahydroacridine-3-carbonyl)piperazine-1-carboxylate (A068)

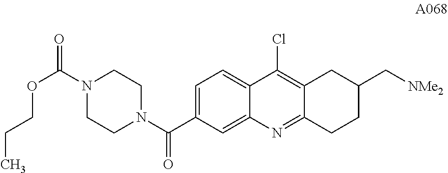 [SMILES: CCCOC(=O)N1CCN(C(=O)c2ccc3c(Cl)c4c(nc3c2)CCC(CN(C)C)C4)CC1]

Compound A068 was prepared according to General Procedure A, B and C2 using 4-((dimethylamino)methyl)cyclohexanone (General Procedure A) and n-propyl piperazine-1-carboxylate (General Procedure C2) as starting materials.

¹H NMR (400 MHz, DMSO-d₆) δ 8.19 (d, J=8.8 Hz, 1H), 7.94 (s, 1H), 7.65 (dd, J=8.8, 1.6 Hz, 1H), 3.98 (t, J=6.8 Hz, 2H), 3.71-3.26 (m, 8H), 3.25-3.20 (m, 1H), 3.21-3.06 (m, 2H), 2.55-2.49 (m, 1H), 2.32-2.03 (m, 10H), 1.61-1.50 (m, 3H), 0.89 (t, J=7.6 Hz, 3H).

LCMS (ESI-TOF) m/z 473.3 [M+H⁺] with a purity of >98%.

### Propyl 4-(9-chloro-5,6,7,8-tetrahydroacridine-3-carbonyl)-2-(2-methoxy-2-oxoethyl)piperazine-1-carboxylate (A069)

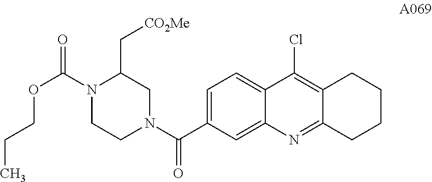 [SMILES: CCCOC(=O)N1CCN(C(=O)c2ccc3c(Cl)c4c(nc3c2)CCCC4)CC1CC(=O)OC]

Step 1: According to General Procedure C1, commercially available 9-chloro-5,6,7,8-tetrahydroacridine-3-carboxylic acid was reacted with tert-butyl 2-(2-methoxy-2-oxoethyl)piperazine-1-carboxylate to give tert-butyl 4-(9-chloro-5,6,7,8-tetrahydroacridine-3-carbonyl)-2-(2-methoxy-2-oxoethyl)piperazine-1-carboxylate.

Step 2: The resulting intermediate (251 mg, 0.50 mmol) was dissolved in trifluoroacetic acid (0.77 mL) and dichloromethane (1.2 mL) for 30 min. The mixture was concentrated and ethyl acetate was added. The organic layer was washed with saturated sodium bicarbonate, brine, dried over anhydrous sodium sulfate, filtered and concentrated under reduced pressure to give methyl 2-(4-(9-chloro-5,6,7,8-tetrahydroacridine-3-carbonyl)piperazin-2-yl)acetate.

Step 3: The crude material from above (201 mg, 0.500 mmol) was dissolved in dichloromethane (2.0 mL) and triethylamine (101.22 mg, 1.00 mmol, 2 equiv) followed by n-propyl chloroformate (91.4 mg, 0.75 mmol, 1.5 equiv) were added at 0° C. After 30 min, the mixture was quenched with saturated ammonium chloride and the organic layer was separated. The aqueous layer was extracted with dichloromethane and the combined organic layer was washed with saturated sodium bicarbonate, brine, dried over anhydrous sodium sulfate, and concentrated under reduced pressure. The crude material was purified by column chromatography (ethyl acetate/hexanes) to afford A069 as a white solid (208 mg, 85%) upon lyophilization.

¹H NMR (400 MHz, 80° C., DMSO-d₆) δ 8.18 (d, J=8.5 Hz, 1H), 7.90 (s, 1H), 7.61 (d, J=8.6 Hz, 1H), 4.47 (s, 1H), 4.15-3.71 (m, 5H), 3.48 (s, 3H), 3.30 (s, 1H), 3.21-3.11 (m, 2H), 3.05 (s, 2H), 3.00 (s, 2H), 2.74-2.51 (m, 2H), 1.91 (s, 4H), 1.64-1.48 (m, 2H), 0.89 (t, J=7.4 Hz, 3H).

LCMS (ESI-TOF) m/z 488.1 [M+H⁺] with a purity of >99%.

### Propyl 4-(9-chloro-5,6,7,8-tetrahydroacridine-3-carbonyl)-3-(2-methoxy-2-oxoethyl)piperazine-1-carboxylate (A070)

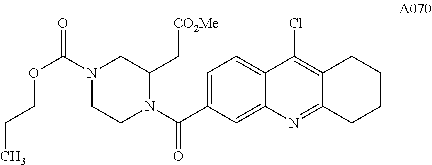 [SMILES: CCCOC(=O)N1CCN(C(=O)c2ccc3c(Cl)c4c(nc3c2)CCCC4)C(CC(=O)OC)C1]

Compound A070 was prepared according to General Procedure C1, using commercially available 9-chloro-5,6,7,8-tetrahydroacridine-3-carboxylic acid and n-propyl 3-(2-methoxy-2-oxoethyl)piperazine-1-carboxylate as starting materials.

¹H NMR (400 MHz, 80° C., DMSO-d₆) δ 8.18 (d, J=8.6 Hz, 1H), 7.91 (s, 1H), 7.60 (d, J=8.5 Hz, 1H), 4.69 (br s, 1H), 4.15-3.69 (m, 5H), 3.59 (s, 3H), 3.23 (dd, J=13.6, 3.6 Hz, 2H), 3.06 (s, 2H), 3.00 (s, 3H), 2.69 (d, J=7.3 Hz, 2H), 1.90 (d, J=3.0 Hz, 4H), 1.64-1.46 (m, 2H), 0.89 (t, J=7.4 Hz, 3H).

LCMS (ESI-TOF) m/z 488.1 [M+H⁺] with a purity of >96%.

### Propyl 4-(9-chloro-5,6,7,8-tetrahydroacridine-3-carbonyl)-3-(2-(dimethylamino)-2-oxoethyl)piperazine-

1-carboxylate (A071) and propyl 4-(9-chloro-5,6,7,8-tetrahydroacridine-3-carbonyl)-3-(2-(methylamino)-2-oxoethyl)piperazine-1-carboxylate (A072)

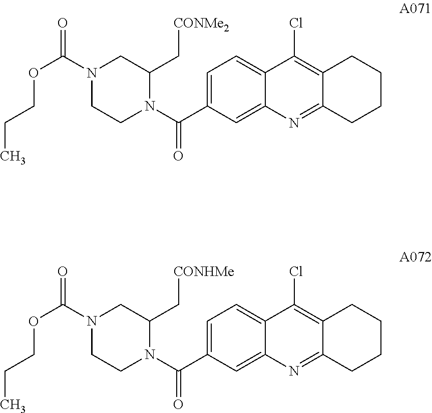

Step 1: To a solution of A070 (153.4 mg, 0.3144 mmol) in methanol (1 mL) and 1,4-dioxane (0.5 mL) was added an aqueous solution of lithium hydroxide (75.3 mg, 3.144 mmol, 10 equiv) in water (1 mL) at 0° C. The reaction was allowed to stir for 30 min before quenching by the addition of concentrated hydrochloric acid and ethyl acetate to pH 2. The organic layer was separated and the aqueous layer was extracted thrice with ethyl acetate. The combined organics were dried over anhydrous sodium sulfate, filtered and concentrated under reduced pressure to afford 2-(1-(9-chloro-5,6,7,8-tetrahydroacridine-3-carbonyl)-4-(propoxycarbonyl)piperazin-2-yl)acetic acid.

Step 2: To a solution of crude intermediate (106.1 mg, 0.2239 mmol) in dichloromethane (2.2 mL) and N,N-dimethylformamide (0.004 mL) was added oxalyl chloride (0.04 mL, 0.466 mmol, 2.08 equiv). When bubbling has ceased (10 min), the suspension was sonicated for 30 min and then stirred for another 1.5 h. The contents were concentrated under reduced pressure. To the resulting residue (36.73 mg, 0.075 mmol) in tetrahydrofuran (1 mL) was added simultaneously triethylamine (0.03 mL, 0.215 mmol, 2.9 equiv) and a 2.0 M solution of dimethylamine (0.05 mL, 0.1 mmol, 1.34 equiv) in ethanol. The mixture was stirred for 3 h before quenching with saturated sodium bicarbonate. The aqueous layer was extracted 3 times with ethyl acetate, the combined organics was dried over anhydrous sodium sulfate, filtered and concentrated under reduced pressure. The crude material was purified by column chromatography (ethyl acetate/hexanes) to afford A071 as a yellow solid (21.5 mg, 58%) upon lyophilization.

In a separate pot, the acid chloride residue (36.73 mg, 0.075 mmol) in tetrahydrofuran (1 mL) was added simultaneously triethylamine (0.03 mL, 0.215 mmol, 2.9 equiv) and a 2.0 M solution of methylamine (0.05 mL, 0.1 mmol, 1.34 equiv) in tetrahydrofuran. The mixture was stirred for 18 h before quenching with saturated sodium bicarbonate. The aqueous layer was extracted 3 times with ethyl acetate, the combined oganics was dried over anhydrous sodium sulfate, filtered and concentrated under reduced pressure. The crude material was purified by preparative HPLC to afford A072 as a white solid (2.2 mg, 6%) upon lyophilization.

A071: ¹H NMR (400 MHz, 80° C., DMSO-d₆) δ 8.17 (d, J=8.6 Hz, 1H), 7.91 (s, 1H), 7.60 (d, J=8.6 Hz, 1H), 4.68 (br s, 1H), 4.07-3.85 (m, 4H), 3.36-3.12 (m, 2H), 3.05 (s, 6H), 3.01-2.67 (m, 8H), 1.90 (d, J=3.1 Hz, 4H), 1.64-1.48 (m, 2H), 0.87 (t, J=7.4 Hz, 3H).

LCMS (ESI-TOF) m/z 501.2 [M+H⁺] with a purity of >96%.

A072: ¹H NMR (400 MHz, 80° C., DMSO-d₆) δ 8.17 (d, J=8.6 Hz, 1H), 7.90 (s, 1H), 7.59 (d, J=8.5 Hz, 1H), 4.63 (s, 1H), 4.05-3.79 (m, 4H), 3.52 (s, 1H), 3.22-2.89 (m, 5H), 2.55 (d, J=4.3 Hz, 3H), 2.44 (d, J=7.3 Hz, 1H), 1.91 (s, 4H), 1.58 (dq, J=14.2, 7.2 Hz, 2H), 0.88 (t, J=7.3 Hz, 3H).

LCMS (ESI-TOF) m/z 487.2 [M+H⁺] with a purity of >94%.

### Propyl (2S)-4-(9-chloro-6-(pyridin-2-yl)-5,6,7,8-tetrahydroacridine-3-carbonyl)-2-methylpiperazine-1-carboxylate (A073)

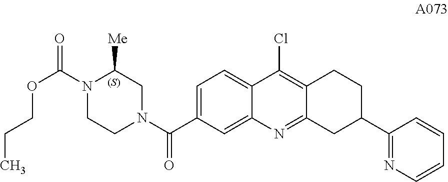

Intermediate from General Procedure F in the synthesis of A053 was subjected to General Procedure C1 with (S)-n-propyl 2-methylpiperazine-1-carboxylate as reagent, followed by General Procedure G to afford A073.

¹H NMR (400 MHz, DMSO-d₆) δ 8.54 (d, J=4.6 Hz, 1H), 8.23 (d, J=8.6 Hz, 1H), 7.96 (s, 1H), 7.78 (td, J=7.7, 1.7 Hz, 1H), 7.68 (s, 1H), 7.43 (d, J=7.9 Hz, 1H), 7.27 (dd, J=6.5, 4.9 Hz, 1H), 4.58-4.04 (m, 2H), 4.03-3.92 (m, 2H), 3.91-3.35 (m, 6H), 3.24-2.83 (m, 4H), 2.30 (d, J=20.9 Hz, 1H), 2.11 (s, 1H), 1.64-1.49 (m, 2H), 1.32-0.92 (m, 3H), 0.88 (t, J=7.4 Hz, 3H).

LCMS (ESI-TOF) m/z 507.2 [M+H⁺] with a purity of >98%.

### Propyl (2R)-4-(9-chloro-6-(pyridin-2-yl)-5,6,7,8-tetrahydroacridine-3-carbonyl)-2-methylpiperazine-1-carboxylate (A074)

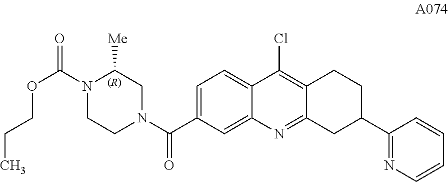

Intermediate from General Procedure F in the synthesis of A053 was subjected to General Procedure C1 with (R)-n-propyl 2-methylpiperazine-1-carboxylate as reagent, followed by General Procedure G to afford A074.

¹H NMR (400 MHz, DMSO-d₆) δ 8.54 (d, J=4.0 Hz, 1H), 8.23 (d, J=8.6 Hz, 1H), 7.96 (s, 1H), 7.78 (td, J=7.7, 1.7 Hz, 1H), 7.68 (s, 1H), 7.43 (d, J=7.8 Hz, 1H), 7.26 (dd, J=7.1, 5.2 Hz, 1H), 4.52-4.03 (m, 2H), 4.03-3.92 (m, 2H), 3.91-3.34 (m, 6H), 3.24-2.85 (m, 4H), 2.36-2.22 (m, 1H), 2.10 (s, 1H), 1.66-1.49 (m, 2H), 1.32-0.94 (m, 3H), 0.88 (t, J=7.4 Hz, 3H).

LCMS (ESI-TOF) m/z 507.2 [M+H⁺] with a purity of >99%.

### Propyl 4-(9-chloro-7-(2-(dimethylamino)ethyl)-5,6,7,8-tetrahydroacridine-3-carbonyl)piperazine-1-carboxylate (A075)

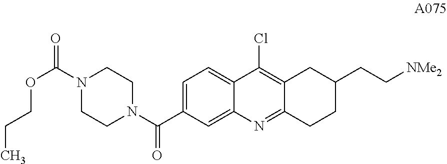 [SMILES: CCCOC(=O)N1CCN(C(=O)c2ccc3c(Cl)c4c(nc3c2)CCC(CCN(C)C)C4)CC1]

Compound A075 was prepared according to General Procedure A, B, C2 using 4-(2-(dimethylamino)ethyl)cyclohexanone (General Procedure A) and n-propyl piperazine-1-carboxylate (General Procedure C2) as starting materials.

¹H NMR (400 MHz, DMSO-d₆) δ 8.19 (d, J=8.4 Hz, 1H), 7.94 (s, 1H), 7.65 (dd, J=8.8, 1.6 Hz, 1H), 3.98 (t, J=6.4 Hz, 2H), 3.66-3.39 (m, 8H), 3.23-3.00 (m, 3H), 2.59-2.54 (m, 1H), 2.38-2.34 (m, 2H), 2.16 (s, 6H), 2.11-2.04 (m, 1H), 1.92 (br s, 1H), 1.61-1.50 (m, 5H), 0.89 (t, J=7.6 Hz, 3H).

LCMS (ESI-TOF) m/z 487.3 [M+H⁺] with a purity of >98%.

### Propyl 4-(6-(2-aminoethyl)-9-chloro-5,6,7,8-tetrahydroacridine-3-carbonyl)piperazine-1-carboxylate (A076)

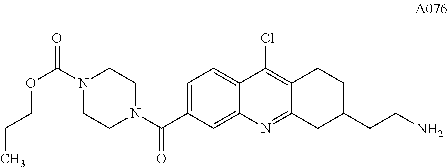 [SMILES: CCCOC(=O)N1CCN(C(=O)c2ccc3c(Cl)c4c(nc3c2)CC(CCN)CC4)CC1]

Compound A076 was prepared using A065 as starting material according to General Procedure D.

¹H NMR (400 MHz, DMSO-d₆) δ 8.19 (d, J=8.8 Hz, 1H), 7.94 (s, 1H), 7.65 (dd, J=8.4, 1.6 Hz, 1H), 3.98 (t, J=8.0 Hz, 2H), 3.66-3.60 (m, 8H), 3.30-3.09 (m, 4H), 2.93-2.84 (m, 1H), 2.74-2.67 (m, 3H), 2.02-1.99 (m, 2H), 1.61-1.45 (m, 5H), 0.89 (t, J=7.2 Hz, 3H).

LCMS (ESI-TOF) m/z 459.3 [M+H⁺] with a purity of >97%.

### Propyl 4-(6-(2-amino-2-oxoethyl)-9-chloro-5,6,7,8-tetrahydroacridine-3-carbonyl)piperazine-1-carboxylate (A077)

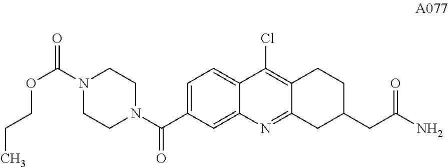 [SMILES: CCCOC(=O)N1CCN(C(=O)c2ccc3c(Cl)c4c(nc3c2)CC(CC(N)=O)CC4)CC1]

Compound A077 was prepared using A065 as starting material in dimethylsulfoxide at 0° C. and potassium carbonate followed by 30% hydrogen peroxide was added. The reaction mixture was stirred at room temperature for 12 h. The reaction mass was diluted with ethyl acetate and the organic layer was washed with water, brine, dried over anhydrous sodium sulfate and concentrated to afford A077.

¹H NMR (400 MHz, DMSO-d₆) δ 8.19 (d, J=8.4 Hz, 1H), 7.94 (s, 1H), 7.65 (dd, J=8.0, 1.6 Hz, 1H), 7.34 (br s, 1H), 6.83 (s, 1H), 3.98 (t, J=6.8 Hz, 2H), 3.66-3.26 (m, 8H), 3.20-3.10 (m, 2H), 2.99-2.86 (m, 1H), 2.79-2.72 (m, 1H), 2.32 (br s, 1H), 2.19-2.17 (m, 2H), 2.06-2.03(m, 1H),1.61-1.55 (m, 3H), 0.89 (t, J=7.6 Hz, 3H).

LCMS (ESI-TOF) m/z 473.3 [M+H⁺] with a purity of >97%.

### Propyl 4-(9-chloro-7-(cyanomethyl)-5,6,7,8-tetrahydroacridine-3-carbonyl)piperazine-1-carboxylate (A078)

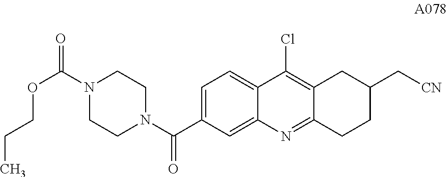 [SMILES: CCCOC(=O)N1CCN(C(=O)c2ccc3c(Cl)c4c(nc3c2)CCC(CC#N)C4)CC1]

Compound A078 was prepared according to General Procedure A, B and C2 using 2-(4-oxocyclohexyl)acetonitrile (General Procedure A) and n-propyl piperazine-1-carboxylate for Step 3 (General Procedure C2) as starting materials.

¹H NMR (400 MHz, DMSO-d₆) δ 8.21 (d, J=8.8 Hz, 1H), 7.95 (s, 1H), 7.67 (dd, J=8.4, 1.6 Hz, 1H), 3.98 (t, J=6.8 Hz, 2H), 3.66-3.27 (m, 8H) 3.16-3.11 (m, 3H), 2.80-2.77 (m, 2H), 2.71-2.64 (m, 1H), 2.32-2.28 (m, 1H), 2.11 (d, J=10.8 Hz, 1H), 1.70-1.55 (m, 3H), 0.89 (t, J=7.6 Hz, 3H).

LCMS (ESI-TOF) m/z 455.2 [M+H⁺] with a purity of >99%.

### Propyl 4-(7-(2-amino-2-oxoethyl)-9-chloro-5,6,7,8-tetrahydroacridine-3-carbonyl)piperazine-1-carboxylate (A079)

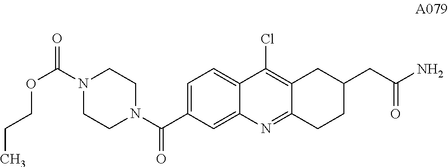 [SMILES: CCCOC(=O)N1CCN(C(=O)c2ccc3c(Cl)c4c(nc3c2)CCC(CC(N)=O)C4)CC1]

To a solution of compound A078 (91 mg, 0.2 mmol) in dimethylsulfoxide (1 mL) was added potassium carbonate (41.4 mg, 0.3 mmol, 1.5 equiv) and 30% hydrogen peroxide (100 μL). The reaction was stirred at room temperature for 24 h before quenching with minimal amount of saturated sodium thiosulfate. The aqueous layer was extracted thrice with ethyl acetate, and the combined organic layers were washed with brine, dried over sodium sulfate, filtered and concentrated. The crude material was purified by column chromatography (methanol/dichloromethane) to afford A079 (50 mg, 53%) as a white solid upon lyophilization.

¹H NMR (400 MHz, DMSO-d₆) δ 8.20 (d, J=8.6 Hz, 1H), 7.94 (s, 1H), 7.65 (d, J=8.7 Hz, 1H), 7.38 (s, 1H), 6.85 (s, 1H), 3.97 (t, J=6.6 Hz, 2H), 3.84-3.35 (m, 8H), 3.25-3.00 (m, 3H), 2.59 (dd, J=17.6, 9.5 Hz, 1H), 2.36-2.16 (m, 3H), 2.03 (d, J=12.4 Hz, 1H), 1.58 (dd, J=14.0, 6.9 Hz, 3H), 0.89 (t, J=7.2 Hz, 3H).

LCMS (ESI-TOF) m/z 473.2 [M+H⁺] with a purity of >98%.

### Propyl 8-(9-chloro-5,6,7,8-tetrahydroacridine-3-carbonyl)-6,8-diazabicyclo[3.2.2]nonane-6-carboxylate (A080)

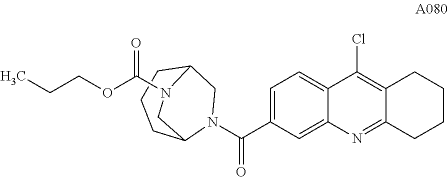 [SMILES: CCCOC(=O)N1CC2CCCC1CN2C(=O)c1ccc2c(Cl)c3c(nc2c1)CCCC3]

Step 1: According to General Procedure C1, commercially available 9-chloro-5,6,7,8-tetrahydroacridine-3-carboxylic acid was reacted with tert-butyl 6,8-diazabicyclo[3.2.2]nonane-6-carboxylate to give tert-butyl 8-(9-chloro-5,6,7,8-tetrahydroacridine-3-carbonyl)-6,8-diazabicyclo[3.2.2]nonane-6-carboxylate.

Step 2: The resulting intermediate (300 mg, 0.638 mmol) was dissolved in trifluoroacetic acid (1.2 mL) and dichloromethane (6 mL) for 24 h. The mixture was diluted with dichloromethane (50 mL) and the organic layer was washed with saturated sodium bicarbonate, brine, dried over anhydrous sodium sulfate, filtered and concentrated under reduced pressure to afford (6,8-diazabicyclo[3.2.2]nonan-6-yl) (9-chloro-5,6,7,8-tetrahydroacridin-3-yl)methanone.

Step 3: The crude material from above (100 mg, 0.27 mmol) was dissolved in dichloromethane (5.0 mL) and triethylamine (58.9 mg, 0.582 mmol, 2.15 equiv) followed by n-propyl chloroformate (53.5 mg, 0.436 mmol, 1.61 equiv) were added at 0° C. After 1 h, the mixture was diluted with ethyl acetate (50 mL) and the organic layer was washed with saturated sodium bicarbonate, brine, dried over anhydrous sodium sulfate, and concentrated under reduced pressure. The crude material was purified by column chromatography (ethyl acetate/hexanes) to afford A080 as a white solid (30 mg, 24%) upon lyophilization.

¹H NMR (400 MHz, 80° C., DMSO-d₆) δ 8.18 (d, J=8.5 Hz, 1H), 7.99 and 7.88 (2×s, 1H), 7.62 (s, 1H), 5.04-3.24 (m, 9H), 3.06 (s, 2H), 2.99 (s, 2H), 2.03-1.71 (m, 6H), 1.66-1.43 (m, 5H), 0.89 (s, 3H).

LCMS (ESI-TOF) m/z 456.2 [M+H⁺] with a purity of >96%.

### Propyl (3S)-4-(7-(2-aminoethyl)-9-chloro-5,6,7,8-tetrahydroacridine-3-carbonyl)-3-methylpiperazine-1-carboxylate (A081)

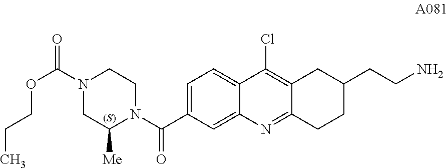

Compound A081 was prepared according to General Procedure A, B, C2 and D using 2-(4-oxocyclohexyl)acetonitrile (General Procedure A) and (S)-n-propyl 3-methylpiperazine-1-carboxylate (General Procedure C2) as starting materials.

¹H NMR (400 MHz, DMSO-d₆) δ 8.18 (d, J=8.8 Hz, 1H), 7.89 (s, 1H), 7.61 (d, J=8.8 Hz, 1H), 4.21-3.75 (m, 5H), 3.19-2.99 (m, 5H), 2.94-2.65 (m, 4H), 2.54-2.43 (m, 1H), 2.03-1.95 (m, 2H), 1.58-1.51 (m, 5H), 1.16 (d, J=4.8 Hz, 3H), 0.89 (t, J=7.6 Hz, 3H).

LCMS (ESI-TOF) m/z 473.6 [M+H⁺] with a purity of >95%.

### Propyl (3S)-4-(9-chloro-7-(2-(dimethylamino)ethyl)-5,6,7,8-tetrahydroacridine-3-carbonyl)-3-methylpiperazine-1-carboxylate (A082)

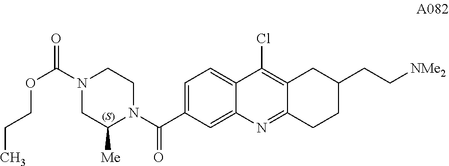

Compound A082 was prepared according to General procedures steps A, B and C2 using 4-(2-(dimethylamino)ethyl)cyclohexanone (General Procedure A) and (S)-n-propyl 3-methylpiperazine-1-carboxylate (General Procedure C2) as starting materials.

¹H NMR (400 MHz, DMSO-d₆) δ 8.19 (d, J=8.8 Hz, 1H), 7.91 (s, 1H), 7.63 (dd, J=8.8, 1.6 Hz, 1H), 4.02-3.77 (m, 5H), 3.26-2.99 (m, 7H), 2.59-2.54 (m, 1H), 2.37-2.32 (m, 2H), 2.15 (s, 6H), 2.10-2.03 (m, 1H), 1.91 (s, 1H), 1.58-1.48 (m, 5H), 1.16 (d, J=4.8 Hz, 3H), 0.89 (t, J=7.6 Hz, 3H).

LCMS (ESI-TOF) m/z 501.3 [M+H⁺] with a purity of >98%.

### (S)-(9-Chloro-5,6,7,8-tetrahydroacridin-3-yl)(2-methyl-4-(5-methylisoxazole-3-carbonyl)piperazin-1-yl)methanone (A083)

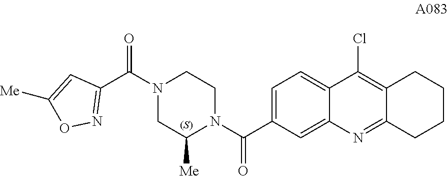

Step 1: 5-methylisoxazole-3-carboxylic acid (20 mg, 0.15 mmol) and tert-butyl (S)-2-methylpiperazine-1-carboxylate (40.1 mg, 0.20 mmol, 1.33 equiv) were dissolved in N,N-dimethylformamide (10 mL) before HATU (190 mg, 0.5 mmol, 3.33 equiv), N,N-diisopropylethylamine (0.35 mL, 2.0 mmol, 13.3 equiv) and 4-dimethylaminopyridine (1 mg) were added. The mixture was stirred for 4 h at room temperature before adding brine and extracting with ethyl acetate. The combined organic layers were dried over anhydrous sodium sulfate, filtered and concentrated under reduced pressure to give tert-butyl (S)-2-methyl-4-(5-methylisoxazole-3-carbonyl)piperazine-1-carboxylate.

Step 2: The crude material was dissolved in dichloromethane (5 mL) and trifluoroacetic acid (5 mL) for 4 h before concentrating. The crude material was purified by column chromatography (ethyl acetate/hexanes) to give (S)-(5-methylisoxazol-3-yl)(3-methylpiperazin-1-yl)methanone (15 mg, 48% over 2 steps).

Step 3: The intermediate from above was subjected to General Procedure C1 with 9-chloro-5,6,7,8-tetrahydroacridine-3-carboxylic acid to afford A083.

¹H NMR (400 MHz, DMSO-d₆) δ 8.21 (d, J=8.5 Hz, 1H), 7.95 (s, 1H), 7.66 (d, J=8.4 Hz, 1H), 6.49 (d, J=21.4 Hz, 1H), 4.58-3.76 (m, 7H), 3.07 (s, 2H), 2.99 (s, 2H), 2.46 (s, 3H), 1.90 (s, 4H), 1.23-1.08 (m, 3H).

LCMS (ESI-TOF) m/z 453.2 [M+H⁺] with purity >96%.

### Propyl (3S)-4-(9-chloro-7-(2-methoxy-2-oxoethyl)-5,6,7,8-tetrahydroacridine-3-carbonyl)-3-methylpiperazine-1-carboxylate (A084)

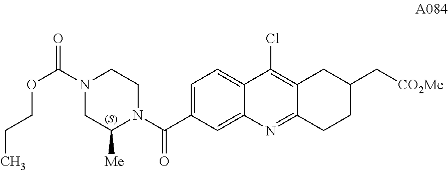

From the intermediate of General Procedure B in the synthesis of A034, General Procedure C1 was conducted with (S)-n-propyl 3-methylpiperazine-1-carboxylate to afford compound A084.

¹H NMR (400 MHz, DMSO-d₆) δ 8.19 (d, J=8.7 Hz, 1H), 7.89 (s, 1H), 7.61 (d, J=8.7 Hz, 1H), 4.33 (s, 1H), 4.03-3.74 (m, 5H), 3.66 (s, 3H), 3.33-2.89 (m, 8H), 2.65 (dd, J=17.3, 10.4 Hz, 1H), 2.31 (s, 1H), 2.06 (d, J=14.6 Hz, 1H), 1.73-1.51 (m, 3H), 1.18 (d, J=6.7 Hz, 3H), 0.89 (t, J=7.4 Hz, 3H).

LCMS (ESI-TOF) m/z 502.2 [M+H⁺] with a purity of >98%.

### Propyl (3R)-4-(9-chloro-7-(2-methoxy-2-oxoethyl)-5,6,7,8-tetrahydroacridine-3-carbonyl)-3-methylpiperazine-1-carboxylate (A085)

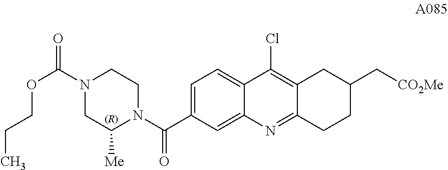

From the intermediate of General Procedure B in the synthesis of A034, General Procedure C1 was conducted with (R)-n-propyl 3-methylpiperazine-1-carboxylate to afford compound A085.

¹H NMR (400 MHz, 80° C., DMSO-d₆) δ 8.19 (d, J=8.6 Hz, 1H), 7.90 (s, 1H), 7.61 (d, J=8.3 Hz, 1H), 4.37-4.28 (m, 1H), 4.04-3.76 (m, 6H), 3.66 (s, 3H), 3.32-3.09 (m, 5H), 3.01-2.91 (m, 1H), 2.65 (dd, J=17.2, 10.4 Hz, 1H), 2.31 (s, 1H), 2.11-2.00 (m, 1H), 1.70-1.51 (m, 4H), 1.18 (d, J=6.8 Hz, 3H), 0.89 (t, J=7.4 Hz, 3H).

LCMS (ESI-TOF) m/z 502.1 [M+H⁺] with a purity of >95%.

### Propyl (3S)-4-(9-chloro-6-(pyridin-2-yl)-5,6,7,8-tetrahydroacridine-3-carbonyl)-3-methylpiperazine-1-carboxylate (A086)

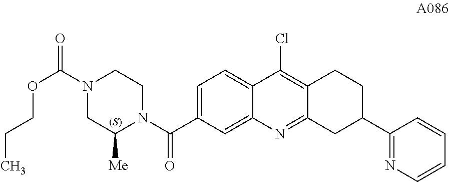

Intermediate from General Procedure F in the synthesis of A053 was subjected to General Procedure C1 with (S)-n-propyl 3-methylpiperazine-1-carboxylate as reagent, followed by General Procedure G to afford A086.

¹H NMR (400 MHz, DMSO-d₆) δ 8.54 (d, J=4.0 Hz, 1H), 8.22 (d, J=8.6 Hz, 1H), 7.95 (d, J=1.2 Hz, 1H), 7.78 (td, J=7.7, 1.8 Hz, 1H), 7.65 (dd, J=8.6, 1.4 Hz, 1H), 7.43 (d, J=7.8 Hz, 1H), 7.29-7.20 (m, 1H), 4.97-3.34 (m, 9H), 3.27-2.73 (m, 5H), 2.35-2.22 (m, 1H), 2.10 (s, 1H), 1.58 (dd, J=14.0, 6.9 Hz, 2H), 1.17 (d, J=5.1 Hz, 3H), 0.89 (t, J=7.3 Hz, 3H).

LCMS (ESI-TOF) m/z 507.2 [M+H⁺] with a purity of >99%.

### Propyl (3R)-4-(9-chloro-6-(pyridin-2-yl)-5,6,7,8-tetrahydroacridine-3-carbonyl)-3-methylpiperazine-1-carboxylate (A087)

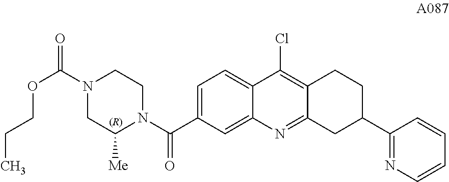

Intermediate from General Procedure F in the synthesis of A053 was subjected to General Procedure C1 with (R)-n-propyl 3-methylpiperazine-1-carboxylate as reagent, followed by General Procedure G to afford A087.

¹H NMR (400 MHz, DMSO-d₆) δ 8.54 (d, J=3.9 Hz, 1H), 8.22 (d, J=8.6 Hz, 1H), 7.95 (s, 1H), 7.78 (t, J=6.9 Hz, 1H), 7.65 (d, J=9.0 Hz, 1H), 7.43 (d, J=7.8 Hz, 1H), 7.32-7.17 (m, 1H), 4.78-3.36 (m, 9H), 3.21-2.86 (m, 5H), 2.28 (s, 1H), 2.10 (s, 1H), 1.65-1.49 (m, 2H), 1.16 (s, 3H), 0.89 (t, J=7.3 Hz, 3H).

LCMS (ESI-TOF) m/z 507.2 [M+H⁺] with a purity of >98%.

### Propyl (3S)-4-(6-(2-aminoethyl)-9-chloro-5,6,7,8-tetrahydroacridine-3-carbonyl)-3-methylpiperazine-1-carboxylate (A088)

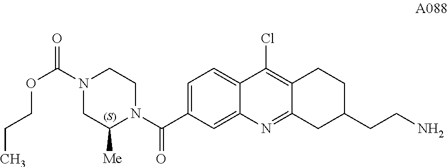

Compound A088 was prepared was prepared according to General procedure steps A, B, C2 and D using 2-(3-oxocyclohexyl)acetamide (General Procedure A) and (S)-n-propyl 3-methylpiperazine-1-carboxylate (General Procedure C2) as starting materials.

¹H NMR (400 MHz, DMSO-d₆) δ 8.20 (d, J=8.8 Hz, 1H), 7.91 (s, 1H), 7.63 (d, J=8.4 Hz, 1H), 4.62-4.10 (m, 3H), 3.99 (t, J=6.4 Hz, 2H), 3.78 (br s, 2H), 3.20-3.11 (m, 5H), 2.94-2.66 (m, 5H), 2.21-2.07 (m, 2H), 1.60-1.52 (m, 5H), 1.16 (m, 3H), 0.89 (t, J=7.2 Hz, 3H).

LCMS (ESI-TOF) m/z 473.3 [M+H⁺] with a purity of >96%.

### Propyl 4-(9-chloro-7-((5-methyl-1,2,4-oxadiazol-3-yl)methyl)-5,6,7,8-tetrahydroacridine-3-carbonyl)piperazine-1-carboxylate (A089)

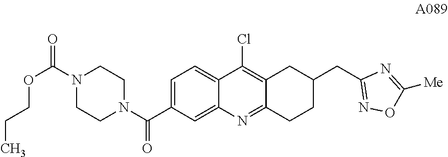 [SMILES: CCCOC(=O)N1CCN(C(=O)c2ccc3c(Cl)c4c(nc3c2)CCC(Cc2noc(C)n2)C4)CC1]

Compound A089 was prepared according to General Procedure A, B and C2 using 4-((5-methyl-1,2,4-oxadiazol-3-yl)methyl)cyclohexanone (General Procedure A) and n-propyl piperazine-1-carboxylate (General Procedure C2) as starting materials.

¹H NMR (400 MHz, DMSO-d₆) δ 8.19 (d, J=8.4 Hz, 1H), 7.94 (s, 1H), 7.65 (d, J=7.2 Hz, 1H), 3.98 (t, J=6.8 Hz, 2H), 3.67-3.26 (m, 8H), 3.22-3.04 (m, 3H), 2.94-2.67 (m, 3H), 2.66 (s, 3H), 2.32 (br s, 1H), 2.04 (d, J=10.8 Hz, 1H), 1.67-1.55 (m, 3H), 0.89 (t, J=7.2 Hz, 3H).

LCMS (ESI-TOF) m/z 512.26 [M+H⁺] with a purity of >99%.

### Propyl (3S)-4-(9-chloro-6-(guanidinomethyl)-5,6,7,8-tetrahydroacridine-3-carbonyl)-3-methylpiperazine-1-carboxylate (A090)

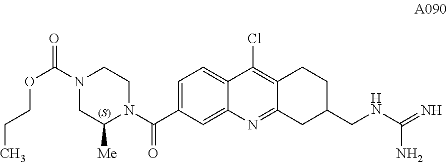

Compound A067 (0.025 mmol) was stirred in N,N-dimethylformamide (2 mL) before 5-methylthioisourea hemisulfate salt (0.2 mmol), N,N-diisopropylethylamine (349 μL, 2.0 mmol) were added and left to react for 2 h at 80° C. Upon cooling, the reaction mixture was filtered to give a yellow solution, which was concentrated to dryness. The crude material was purified by preparative HPLC to obtain compound A090 as a yellow oil (trifluoroacetate salt) (28%).

LCMS (ESI-TOF) m/z 501.2 [M+H⁺] with a purity of >95%.

### Propyl (3S)-4-(9-chloro-6-(2-guanidinoethyl)-5,6,7,8-tetrahydroacridine-3-carbonyl)-3-methylpiperazine-1-carboxylate (A091)

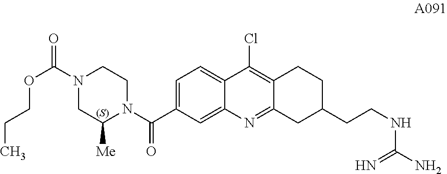

Compound A088 (0.025 mmol) was stirred in N,N-dimethylformamide (2 mL) before 5-methylthioisourea hemisulfate salt (0.2 mmol), N,N-diisopropylethylamine (349 μL, 2.0 mmol) were added and left to react for 2 h at 80° C. Upon cooling, the reaction mixture was filtered to give a yellow solution, which was concentrated to dryness. The crude material was purified by preparative HPLC to obtain compound A091 as a yellow oil (trifluoroacetate salt) (39%). ¹H NMR (400 MHz, CD₃OD) δ 8.42 (d, J=8.7 Hz, 1H), 8.02 (s, 1H), 7.75 (dd, J=8.6, 1.2 Hz, 1H), 4.36-3.79 (m, 5H), 3.59-3.13 (m, 7H), 3.10-2.83 (m, 3H), 2.28-2.16 (m, 1H), 2.15-2.01 (m, 1H), 1.78 (dd, J=14.4, 7.1 Hz, 2H), 1.73-1.59 (m, 3H), 1.31 (d, J=5.9 Hz, 3H), 0.96 (t, J=7.4 Hz, 3H).

LCMS (ESI-TOF) m/z 515.3 [M+H⁺] with a purity of >98%.

### Propyl (3S)-4-(9-chloro-6-(pyridin-4-yl)-5,6,7,8-tetrahydroacridine-3-carbonyl)-3-methylpiperazine-1-carboxylate (A092)

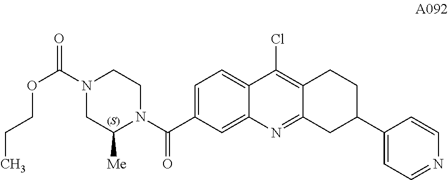

Intermediate from General Procedure F in the synthesis of A058 was subjected to General Procedure C1 with (S)-n-propyl 3-methylpiperazine-1-carboxylate as reagent, followed by General Procedure G to afford A092.

¹H NMR (400 MHz, 80° C., DMSO-d₆) δ 8.52 (d, J=5.3 Hz, 2H), 8.22 (d, J=8.6 Hz, 1H), 7.93 (s, 1H), 7.63 (d, J=8.6 Hz, 1H), 7.37 (d, J=5.2 Hz, 2H), 4.33 (s, 1H), 4.07-3.70 (m, 5H), 3.44-2.90 (m, 8H), 2.26 (s, 1H), 2.07 (s, 1H), 1.66-1.44 (m, 2H), 1.18 (d, J=6.7 Hz, 3H), 0.89 (t, J=7.4 Hz, 3H).

LCMS (ESI-TOF) m/z 507.2 [M+H⁺] with a purity of >99%.

### Propyl (3R)-4-(9-chloro-6-(pyridin-4-yl)-5,6,7,8-tetrahydroacridine-3-carbonyl)-3-methylpiperazine-1-carboxylate (A093)

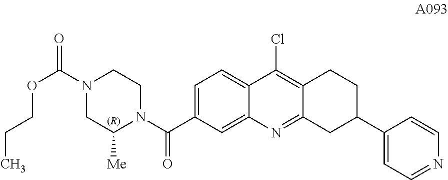

Intermediate from General Procedure F in the synthesis of A058 was subjected to General Procedure C1 with (R)-n-propyl 3-methylpiperazine-1-carboxylate as reagent, followed by General Procedure G to afford A093.

¹H NMR (400 MHz, 80° C., DMSO-d₆) δ 8.52 (d, J=5.2 Hz, 2H), 8.22 (d, J=8.6 Hz, 1H), 7.93 (s, 1H), 7.63 (d, J=8.5 Hz, 1H), 7.37 (d, J=5.3 Hz, 2H), 4.33 (s, 1H), 4.06-3.72 (m, 5H), 3.43-2.93 (m, 8H), 2.25 (s, 1H), 2.07 (s, 1H), 1.65-1.50 (m, 2H), 1.18 (d, J=6.7 Hz, 3H), 0.89 (t, J=7.4 Hz, 3H).

LCMS (ESI-TOF) m/z 507.2 [M+H⁺] with a purity of >98%.

### Propyl 4-(9-chloro-6-(5-fluoropyridin-2-yl)-5,6,7,8-tetrahydroacridine-3-carbonyl)piperazine-1-carboxylate (A094)

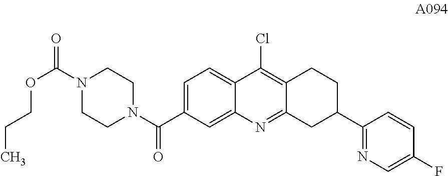 [SMILES: CCCOC(=O)N1CCN(C(=O)c2ccc3c(Cl)c4c(nc3c2)CC(c2ccc(F)cn2)CC4)CC1]

Compound A094 was prepared according to General Procedure E, F, C2 and G using 3-(5-fluoropyridin-2-yl)cyclohexanone (General Procedure E) and n-propyl piperazine-1-carboxylate (General Procedure C2) as starting materials.

¹H NMR (400 MHz, DMSO-d₆) δ 8.53 (d, J=3.2 Hz, 1H), 8.22 (d, J=8.8 Hz, 1H), 7.97 (s, 1H), 7.75-7.66 (m, 2H), 7.54-7.51 (m, 1H), 3.98 (t, J=6.6 Hz, 2H), 3.67-3.29 (m, 11H), 3.18-3.12 (m, 1H), 3.09-3.00 (m, 1H), 2.33-2.26 (m, 1H), 2.10-2.06 (m, 1H), 1.60-1.55 (m, 2H), 0.89 (t, J=6.8 Hz, 3H).

LCMS (ESI-TOF) m/z 511.3 [M+H⁺] with a purity of >98%.

### Propyl (3S)-4-(9-chloro-6-(5-fluoropyridin-2-yl)-5,6,7,8-tetrahydroacridine-3-carbonyl)-3-methylpiperazine-1-carboxylate (A095)

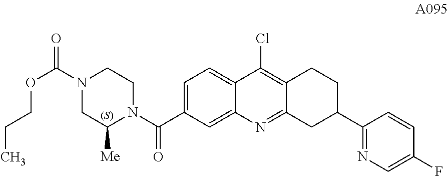

Intermediate from General Procedure F in the synthesis of A094 was subjected to General Procedure C2 with (S)-n-propyl 3-methylpiperazine-1-carboxylate as reagent, followed by General Procedure G to afford A095.

¹H NMR (400 MHz, DMSO-d₆) δ 8.53 (d, J=3.2 Hz, 1H), 8.22 (d, J=8 Hz, 1H), 7.95 (s, 1H), 7.75-7.64 (m, 2H), 7.54-7.51 (m, 1H), 3.98-3.79 (m, 5H), 3.50-3.28 (m, 4H), 3.18-3.0 (m, 5H), 2.32-2.26 (m, 1H), 2.06 (m,1H), 1.57 (d, J=6.8 Hz, 2H), 1.16 (m, 3H), 0.89 (t, J=7.2 Hz, 3H).

LCMS (ESI-TOF) m/z 525.6 [M+H⁺] with a purity of >97%.

### Propyl 4-(9-chloro-6-(3-methylpyridin-2-yl)-5,6,7,8-tetrahydroacridine-3-carbonyl)piperazine-1-carboxylate (A096)

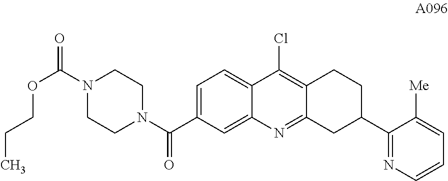 [SMILES: CCCOC(=O)N1CCN(C(=O)c2ccc3c(Cl)c4c(nc3c2)CC(c2ncccc2C)CC4)CC1]

Compound A096 was prepared according to General Procedure E, F, C2 and G using 3-(3-methylpyridin-2-yl)cyclohexanone (General Procedure E) and n-propyl piperazine-1-carboxylate (General Procedure C1) as starting materials.

¹H NMR (400 MHz, DMSO-d₆) δ 8.35 (d, J=4.4 Hz, 1H), 8.22 (d, J=8.4 Hz, 1H), 7.97 (s, 1H), 7.67 (d, J=8.8 Hz, 1H), 7.59 (d, J=6.8 Hz, 1H), 7.18-7.15 (m, 1H), 3.98 (t, J=6.4 Hz, 2H), 3.67-3.35 (m, 10H), 3.25-3.07 (m, 4H), 2.39 (s, 3H), 2.13-2.07 (m, 2H), 1.59-1.55 (m, 2H), 0.89 (t, J=7.2 Hz, 3H).

LCMS (ESI-TOF) m/z 507.6 [M+H⁺] with a purity of >96%.

### Propyl (2S)-4-(9-chloro-6-(5-fluoropyridin-2-yl)-5,6,7,8-tetrahydroacridine-3-carbonyl)-2-methylpiperazine-1-carboxylate (A097)

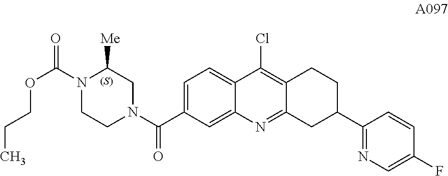

Intermediate from General Procedure F in the synthesis of A094 was subjected to General Procedure C1 with (S)-n-propyl 2-methylpiperazine-1-carboxylate as reagent, followed by General Procedure G to afford A097.

¹H NMR (400 MHz, DMSO-d₆) δ 8.53 (d, J=2.9 Hz, 1H), 8.23 (d, J=8.6 Hz, 1H), 7.96 (s, 1H), 7.77-7.62 (m, 2H), 7.52 (dd, J=8.7, 4.5 Hz, 1H), 4.54-3.34 (m, 10H), 3.25-2.88 (m, 4H), 2.27 (s, 1H), 2.09 (s, 1H), 1.65-1.51 (m, 2H), 1.29-0.94 (m, 3H), 0.88 (t, J=7.4 Hz, 3H).

LCMS (ESI-TOF) m/z 525.2 [M+H⁺] with a purity of >99%.

### Propyl (2R)-4-(9-chloro-6-(5-fluoropyridin-2-yl)-5,6,7,8-tetrahydroacridine-3-carbonyl)-2-methylpiperazine-1-carboxylate (A098)

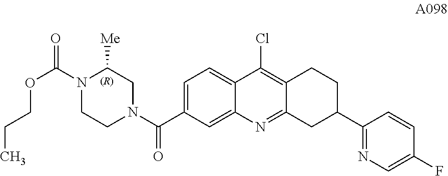

Intermediate from General Procedure F in the synthesis of A094 was subjected to General Procedure C1 with (R)-n-propyl 2-methylpiperazine-1-carboxylate as reagent, followed by General Procedure G to afford A098.

¹H NMR (400 MHz, DMSO-d₆) δ 8.53 (d, J=2.9 Hz, 1H), 8.23 (d, J=8.6 Hz, 1H), 7.96 (br s, 1H), 7.77-7.61 (m, 2H), 7.52 (dd, J=8.7, 4.5 Hz, 1H), 4.53-3.35 (m, 9H), 3.24-2.86 (m, 5H), 2.35-2.20 (m, 1H), 2.16-1.98 (m, 1H), 1.65-1.50 (m, 2H), 1.29-0.95 (m, 3H), 0.88 (t, J=7.4 Hz, 3H).

LCMS (ESI-TOF) m/z 525.2 [M+H⁺] with a purity of >99%.

### Propyl 4-(9-chloro-6-(3-fluoropyridin-2-yl)-5,6,7,8-tetrahydroacridine-3-carbonyl)piperazine-1-carboxylate (A099)

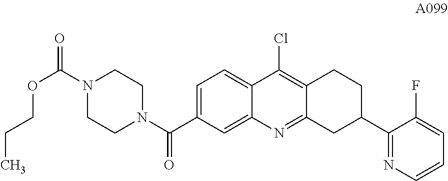 [SMILES: CCCOC(=O)N1CCN(C(=O)c2ccc3c(Cl)c4c(nc3c2)CC(c2ncccc2F)CC4)CC1]

Compound A099 was prepared according to General Procedure E, F, C2 and G using 3-(3-fluoropyridin-2-yl) cyclohexanone (General Procedure E) and n-propyl piperazine-1-carboxylate (General Procedure C2) as starting materials.

¹H NMR (400 MHz, DMSO-d₆) δ 8.38 (d, J=4.4 Hz, 1H), 8.22 (d, J=8.8 Hz, 1H), 7.97 (s, 1H), 7.74-7.66 (m, 2H), 7.41-7.37 (m, 1H), 3.98 (t, J=6.4 Hz, 2H), 3.71-3.38 (m, 10H), 3.19-3.08 (m, 3H), 2.23-2.12 (m, 2H), 1.61-1.55 (m, 2H), 0.89 (t, J=7.2 Hz, 3H).

LCMS (ESI-TOF) m/z 511.3 [M+H⁺] with a purity of >97%.

### Propyl (3S)-4-(9-chloro-6-(3-fluoropyridin-2-yl)-5,6,7,8-tetrahydroacridine-3-carbonyl)-3-methylpiperazine-1-carboxylate (A100)

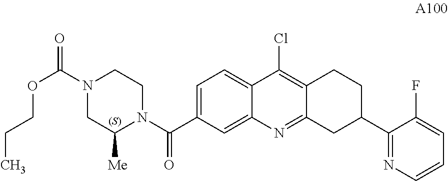

Intermediate from General Procedure F in the synthesis of A099 was subjected to General Procedure C2 with (S)-n-propyl 3-methylpiperazine-1-carboxylate as reagent, followed by General Procedure G to afford A100.

¹H NMR (400 MHz, DMSO-d₆) δ 8.39 (d, J=4.4 Hz, 1H), 8.22 (d, J=8.4 Hz, 1H), 7.94 (s, 1H), 7.74-7.64 (m, 2H), 7.41-7.37 (m, 1H), 3.99-3.71 (m, 7H), 3.49-3.42 (m, 1H), 3.16-2.97 (m, 6H), 2.27-2.26 (m, 1H), 2.14-2.12 (m, 1H), 1.60-1.55 (m, 2H), 1.17 (br s, 3H) 0.88 (t, J=7.2 Hz, 3H).

LCMS (ESI-TOF) m/z 525.2 [M+H⁺] with a purity of >99%.

### Propyl (R/S)-4-(9-chloro-6-(pyridin-2-yl)-5,6,7,8-tetrahydroacridine-3-carbonyl)piperazine-1-carboxylate (A053 (Ent-1/2))

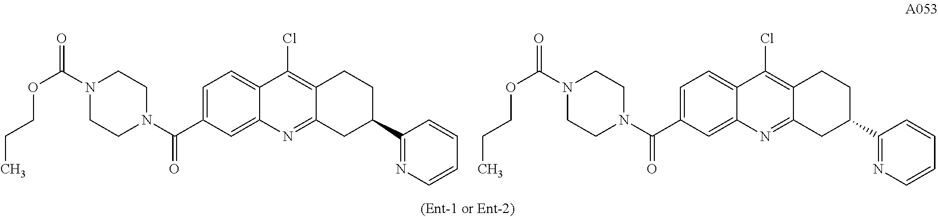

Compound A053(Ent-1) was isolated from SFC purification of A053.

¹H NMR (400 MHz, DMSO-d₆) δ 8.55 (d, J=4.8 Hz, 1H), 8.20 (d, J=8.4 Hz, 1H), 7.96 (s, 1H), 7.84-7.69 (m, 1H) 7.66 (dd, J=1.6, 8.8 Hz, 1H), 7.46 (d, J=7.2 Hz, 1H), 7.34-7.26 (m, 1H), 3.96 (t, J=6.6 Hz, 2H), 3.70-3.00 (m, 13H), 2.27 (br s, 1H), 2.15-2.05 (m, 1H), 1.65-1.50 (m, 2H), 0.87 (t, J=7.2 Hz, 3H).

LCMS (ESI-TOF) m/z 493.3 [M+H⁺] with a purity of >98%.

Compound A053 (Ent-2) was isolated from SFC purification of A053.

¹H NMR (400 MHz, DMSO-d₆) δ 8.54 (d, J=4.0 Hz, 1H), 8.22 (d, J=8.4 Hz, 1H), 7.98 (s, 1H), 7.78 (t, J=7.6 Hz, 1H) 7.67 (d, J=8.8 Hz, 1H), 7.42 (d, J=7.6 Hz, 1H), 7.26 (t, J=6.0 Hz, 1H), 3.97 (t, J=6.6 Hz, 2H), 3.75-3.00 (m, 13H), 2.28 (br s, 1H), 2.11 (br s, 1H), 1.65-1.52 (m, 2H), 0.89 (t, J=7.2 Hz, 3H).

LCMS (ESI-TOF) m/z 493.3 [M+H⁺] with a purity of >99%.

### Propyl (3S)-4-(9-chloro-6-(3-methylpyridin-2-yl)-5,6,7,8-tetrahydroacridine-3-carbonyl)-3-methylpiperazine-1-carboxylate (A101)

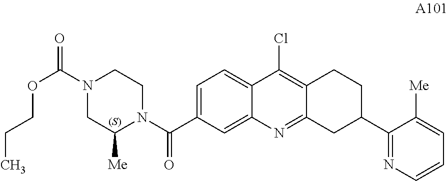

Intermediate from General Procedure F in the synthesis of A096 was subjected to General Procedure C1 with (S)-n-propyl 3-methylpiperazine-1-carboxylate as reagent, followed by General Procedure G to afford A101.

¹H NMR (400 MHz, DMSO-d₆) δ 8.36 (d, J=4.4 Hz, 1H), 8.22 (d, J=8.8 Hz, 1H), 7.94 (s, 1H), 7.66-7.59 (m, 2H), 7.18-7.15 (m, 1H), 3.99-3.77 (m, 5H), 3.57-3.54 (m, 1H), 3.48-3.35 (m, 1H), 3.30-2.97 (m, 7H), 2.39 (s, 3H), 2.14-2.07 (m, 2H), 1.60-1.55 (m, 2H),1.17 (s, 3H), 0.89 (t, J=7.2 Hz, 3H).

LCMS (ESI-TOF) m/z 521.3 [M+H⁺] with a purity of >99%.

### Propyl (3R)-4-(6-(aminomethyl)-9-chloro-5,6,7,8-tetrahydroacridine-3-carbonyl)-3-methylpiperazine-1-carboxylate (A102)

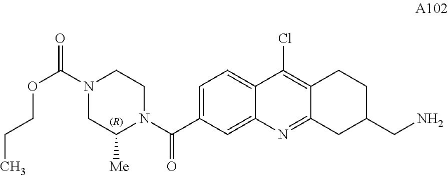

Compound A102 was prepared according to General Procedure A, B, C2 and D using 2-(3-oxocyclohexyl)acetamide (General Procedure A) and (R)-n-propyl 3-methylpiperazine-1-carboxylate (General Procedure C2) as starting materials.

¹H NMR (400 MHz, DMSO-d₆) δ 8.19 (d, J=8.0 Hz, 1H), 7.91 (s, 1H), 7.63 (d, J=8.4 Hz, 1H), 3.98-3.97 (m, 2H), 3.79 (br s, 1H), 3.30-2.87 (m, 7H), 2.75-2.63 (m, 3H), 2.49 (m, 2H), 2.15-2.08 (m, 1H), 1.92-1.61 (m, 1H), 1.58-1.56 (m, 3H), 1.16 (m, 3H), 0.89 (t, J=6.8 Hz, 3H).

LCMS (ESI-TOF) m/z 459.31 [M+H⁺] with a purity of >95%.

### Propyl 4-(9-chloro-6-(pyridin-2-ylmethyl)-5,6,7,8-tetrahydroacridine-3-carbonyl)piperazine-1-carboxylate (A103)

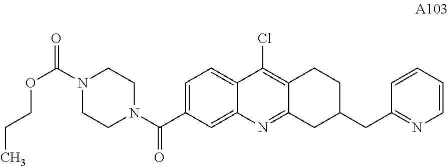 [SMILES: CCCOC(=O)N1CCN(C(=O)c2ccc3c(Cl)c4c(nc3c2)CC(Cc2ccccn2)CC4)CC1]

Compound A103 was prepared according to General Procedure E, F, C2 and G using 3-(pyridin-2-ylmethyl)cyclohexanone (General Procedure E) and n-propyl piperazine-1-carboxylate (General Procedure C2) as starting materials.

¹H NMR (400 MHz, DMSO-d₆) δ 8.53 (d, J=4.4 Hz, 1H), 8.18 (d, J=8.4 Hz, 1H), 7.91 (s, 1H), 7.76-7.72 (m, 1H), 7.64 (d, J=8.0 Hz, 1H), 7.32 (d, J=8.0 Hz, 1H), 7.25-7.22 (m, 1H), 3.98 (t, J=6.4 Hz, 2H), 3.65-3.26 (m, 8H), 3.17-3.12 (m, 1H), 3.07-3.02 (m, 1H), 2.93-2.78 (m, 4H), 2.42-2.40 (m,1H), 2.05-2.02 (m, 1H), 1.64-1.55 (m, 3H), 0.89 (t, J=7.2 Hz, 3H).

LCMS (ESI-TOF) m/z 507.3 [M+H⁺] with a purity of >99%.

### Propyl 4-(9-chloro-6-(2-methylpyridin-4-yl)-5,6,7,8-tetrahydroacridine-3-carbonyl)piperazine-1-carboxylate (A104)

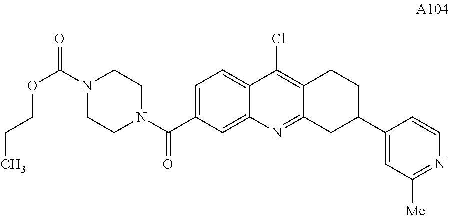 [SMILES: CCCOC(=O)N1CCN(C(=O)c2ccc3c(Cl)c4c(nc3c2)CC(c2ccnc(C)c2)CC4)CC1]

Compound A104 was prepared according to the General Procedure E, F, C2 and G using 3-(2-methylpyridin-4-yl)cyclohexanone (General Procedure E) and n-propyl piperazine-1-carboxylate (General Procedure C2) as starting materials.

¹H NMR (400 MHz, DMSO-d₆) δ 8.39 (d, J=5.6 Hz, 1H), 8.23 (d, J=8.4 Hz, 1H), 7.97 (s, 1H), 7.68 (d, J=8.4 Hz, 1H), 7.27 (s, 1H), 7.19 (d, J=5.2 Hz, 1H), 3.98 (t, J=6.8 Hz, 2H), 3.66-3.26 (m, 7H), 3.20-2.99 (m, 6H), 3.47 (s, 3H), 2.21 (br s, 1H), 2.07-2.01 (m, 1H), 1.61-1.55 (m, 2H), 0.89 (t, J=7.2 Hz, 3H).

LCMS (ESI-TOF) m/z 507.3 [M+H⁺] with a purity of >99%.

### Propyl 4-(9-chloro-6-(2-(dimethylamino)ethyl)-5,6,7,8-tetrahydroacridine-3-carbonyl)piperazine-1-carboxylate (A105)

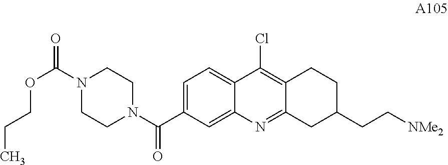 [SMILES: CCCOC(=O)N1CCN(C(=O)c2ccc3c(Cl)c4c(nc3c2)CC(CCN(C)C)CC4)CC1]

Compound A105 was prepared according to General Procedure A, B and C2 using 3-(2-(dimethylamino)ethyl)cyclohexanone (General Procedure A) and n-propyl piperazine-1-carboxylate (General Procedure C2) as starting materials.

¹H NMR (400 MHz, DMSO-d₆) δ 8.19 (d, J=8.8 Hz, 1H), 7.94 (s, 1H), 7.65 (d, J=8.8 Hz, 1H), 3.97 (t, J=6.4 Hz, 2H), 3.70-3.35 (m, 8H), 3.20-2.92 (m, 2H), 2.92-2.83 (m, 1H), 2.76-2.69 (m, 1H), 2.37-2.31 (m, 2H), 2.14 (s, 6H), 2.07-2.04 (m, 1H), 1.94-1.93 (m, 1H) 1.60-1.47 (m, 5H), 0.95 (t, J=7.6 Hz, 3H).

LCMS (ESI-TOF) m/z 487.2 [M+H⁺] with a purity of >96%.

### Propyl (3R)-4-(9-chloro-6-(5-fluoropyridin-2-yl)-5,6,7,8-tetrahydroacridine-3-carbonyl)-3-methylpiperazine-1-carboxylate (A106)

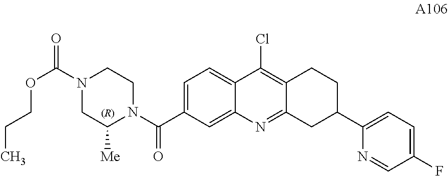

Intermediate from General Procedure F in the synthesis of A094 was subjected to General Procedure C1 with (R)-n-propyl 3-methylpiperazine-1-carboxylate as reagent, followed by General Procedure G to afford A106.

¹H NMR (400 MHz, 80° C., DMSO-d₆) δ 8.48 (d, J=2.5 Hz, 1H), 8.21 (d, J=8.5 Hz, 1H), 7.92 (s, 1H), 7.71-7.57 (m, 2H), 7.48 (dd, J=8.6, 4.4 Hz, 1H), 4.34 (s, 1H), 4.09-3.70 (m, 5H), 3.49-3.29 (m, 3H), 3.27-3.12 (m, 3H), 3.12-2.91 (m, 2H), 2.39-2.19 (m, 1H), 2.18-2.00 (m, 1H), 1.71-1.50 (m, 2H), 1.18 (d, J=6.7 Hz, 3H), 0.89 (t, J=7.3 Hz, 3H).

LCMS (ESI-TOF) m/z 525.2 [M+H⁺] with a purity of >99%.

### Propyl (2S)-4-(9-chloro-6-(3-methylpyridin-2-yl)-5,6,7,8-tetrahydroacridine-3-carbonyl)-2-methylpiperazine-1-carboxylate (A107)

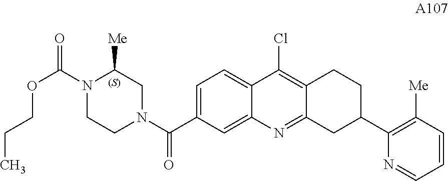

Intermediate from General Procedure F in the synthesis of A096 was subjected to General Procedure C1 with (S)-n-propyl 2-methylpiperazine-1-carboxylate as reagent, followed by General Procedure G to afford A107.

¹H NMR (400 MHz, 80° C., DMSO-d₆) δ 8.34 (d, J=4.6 Hz, 1H), 8.22 (d, J=8.5 Hz, 1H), 7.93 (s, 1H), 7.64 (d, J=8.5 Hz, 1H), 7.56 (d, J=7.6 Hz, 1H), 7.13 (dd, J=7.4, 4.8 Hz, 1H), 4.42-3.67 (m, 6H), 3.65-3.37 (m, 2H), 3.37-3.07 (m, 6H), 2.39 (s, 3H), 2.22-1.98 (m, 2H), 1.64-1.53 (m, 2H), 1.11 (d, J=6.2 Hz, 3H), 0.89 (t, J=7.4 Hz, 3H).

LCMS (ESI-TOF) m/z 521.2 [M+H⁺] with a purity of >99%.

### Propyl (2R)-4-(9-chloro-6-(3-methylpyridin-2-yl)-5,6,7,8-tetrahydroacridine-3-carbonyl)-2-methylpiperazine-1-carboxylate (A108)

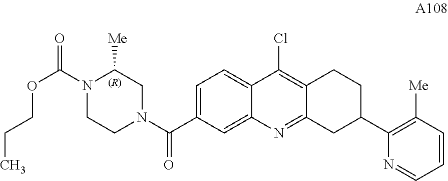

Intermediate from General Procedure F in the synthesis of A096 was subjected to General Procedure C1 with (R)-n-propyl 2-methylpiperazine-1-carboxylate as reagent, followed by General Procedure G to afford A108.

¹H NMR (400 MHz, 80° C., DMSO-d₆) δ 8.34 (d, J=4.6 Hz, 1H), 8.22 (d, J=8.5 Hz, 1H), 7.93 (s, 1H), 7.64 (d, J=8.5 Hz, 1H), 7.56 (d, J=7.6 Hz, 1H), 7.13 (dd, J=7.4, 4.8 Hz, 1H), 4.42-3.67 (m, 6H), 3.65-3.37 (m, 2H), 3.37-3.07 (m, 6H), 2.39 (s, 3H), 2.22-1.98 (m, 2H), 1.64-1.53 (m, 2H), 1.11 (d, J=6.2 Hz, 3H), 0.89 (t, J=7.4 Hz, 3H).

LCMS (ESI-TOF) m/z 521.2 [M+H⁺] with a purity of >99%.

### Propyl (3R)-4-(9-chloro-6-(3-methylpyridin-2-yl)-5,6,7,8-tetrahydroacridine-3-carbonyl)-3-methylpiperazine-1-carboxylate (A109)

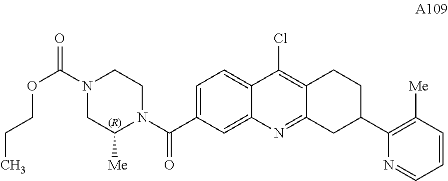

Intermediate from General Procedure F in the synthesis of A096 was subjected to General Procedure C1 with (R)-n-propyl 3-methylpiperazine-1-carboxylate as reagent, followed by General Procedure G to afford A109.

¹H NMR (400 MHz, 80° C., DMSO-d₆) δ 8.34 (d, J=4.4 Hz, 1H), 8.21 (d, J=8.6 Hz, 1H), 7.91 (s, 1H), 7.62 (d, J=8.6 Hz, 1H), 7.56 (d, J=7.4 Hz, 1H), 7.13 (dd, J=7.5, 4.8 Hz, 1H), 4.34 (br s, 1H), 4.06-3.71 (m, 5H), 3.58 (dd, J=12.3, 7.8 Hz, 1H), 3.46 (dd, J=17.1, 10.5 Hz, 1H), 3.20 (dd, J=24.6, 14.2 Hz, 4H), 3.13-2.91 (m, 2H), 2.39 (s, 3H), 2.23-2.02 (m, 2H), 1.65-1.53 (m, 2H), 1.19 (d, J=6.7 Hz, 3H), 0.89 (t, J=7.4 Hz, 3H).

LCMS (ESI-TOF) m/z 521.2 [M+H⁺] with a purity of >99%.

### Propyl 4-(9-chloro-5,6,7,8-tetrahydroacridine-3-carbonyl)-2-(methoxymethyl)piperazine-1-carboxylate (A110)

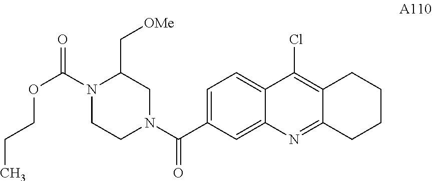 [SMILES: CCCOC(=O)N1CCN(C(=O)c2ccc3c(Cl)c4c(nc3c2)CCCC4)CC1COC]

Step 1: According to General Procedure C1, commercially available 9-chloro-5,6,7,8-tetrahydroacridine-3-carboxylic acid was reacted with tert-butyl 2-(methoxymethyl)piperazine-1-carboxylate to give tert-butyl 4-(9-chloro-5,6,7,8-tetrahydroacridine-3-carbonyl)-2-(methoxymethyl)piperazine-1-carboxylate.

Step 2: The resulting intermediate (150 mg, 0.316 mmol) was dissolved in trifluoroacetic acid (1.2 mL) and dichloromethane (6 mL) for 24 h. The mixture was diluted with dichloromethane (50 mL) and the organic layer was washed with saturated sodium bicarbonate, brine, dried over anhydrous sodium sulfate, filtered and concentrated under reduced pressure to give (9-chloro-5,6,7,8-tetrahydroacridin-3-yl)(3-(methoxymethyl)piperazin-1-yl)methanone.

Step 3: The crude material from above (100 mg, 0.267 mmol) was dissolved in dichloromethane (5.0 mL) and triethylamine (54.1 mg, 0.535 mmol, 2 equiv) followed by n-propyl chloroformate (49.2 mg, 0.401 mmol, 1.5 equiv) were added at 0° C. After 1 h, the mixture was diluted with ethyl acetate (50 mL) and the organic layer was washed with saturated sodium bicarbonate, brine, dried over anhydrous sodium sulfate, and concentrated under reduced pressure. The crude material was purified by column chromatography (ethyl acetate/hexanes) to afford A110 as a white solid (100 mg, 81%) upon lyophilization.

¹H NMR (400 MHz, DMSO-d₆) δ 8.19 (d, J=8.6 Hz, 1H), 7.93 (s, 1H), 7.67 (dd, J=21.4, 7.8 Hz, 1H), 4.64-3.39 (m, 9H), 3.22-2.84 (m, 9H), 1.90 (t, J=2.9 Hz, 4H), 1.58 (dq, J=14.1, 7.0 Hz, 2H), 0.88 (dd, J=9.5, 5.3 Hz, 3H).

LCMS (ESI-TOF) m/z 460.2 [M+H⁺] with a purity of >99%.

### Propyl (3R)-4-(6-(2-aminoethyl)-9-chloro-5,6,7,8-tetrahydroacridine-3-carbonyl)-3-methylpiperazine-1-carboxylate (A111)

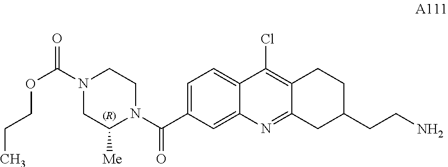

Compound A111 was prepared was prepared according to General procedure steps A, B, C2 and D using 2-(3-oxocyclohexyl)acetamide (General Procedure A) and (R)-n-propyl 3-methylpiperazine-1-carboxylate (General Procedure C2) as starting materials.

¹H NMR (400 MHz, DMSO-d₆) δ 8.20 (d, J=8.4 Hz, 1H), 7.91 (s, 1H), 7.63 (d, J=8.4 Hz, 1H), 4.61-3.71 (m, 6H), 3.17-3.11 (m, 4H), 2.94-2.85 (m, 2H), 2.74-2.66 (m, 3H), 2.06-1.98 (m, 2H), 1.61-1.48 (m, 5H), 1.17 (br s, 3H), 0.89 (t, J=7.2 Hz, 3H).

LCMS (ESI-TOF) m/z 473.42 [M+H⁺] with a purity of >96%.

### Propyl 4-(9-chloro-6-(pyrazin-2-yl)-5,6,7,8-tetrahydroacridine-3-carbonyl)piperazine-1-carboxylate (A112)

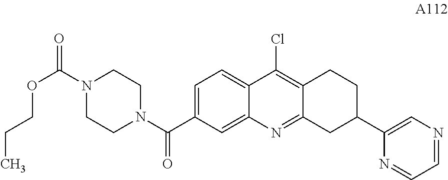 [SMILES: CCCOC(=O)N1CCN(C(=O)c2ccc3c(Cl)c4c(nc3c2)CC(c2cnccn2)CC4)CC1]

Compound A112 was prepared according to General Procedure E, F, C2 and G using 3-(pyrazin-2-yl)cyclohexan-1-one (General Procedure E) and n-propyl piperazine-1-carboxylate (General Procedure C2) as starting materials.

¹H NMR (400 MHz, DMSO-d₆) δ 8.76 (d, J=0.8 Hz, 1H), 8.62-8.61 (m, 1H), 8.55 (d, J=2.4 Hz, 1H), 8.23 (d, J=8.4 Hz, 1H), 7.98 (d, J=1.2 Hz, 1H), 7.68 (dd, J=8.4, 1.6 Hz, 1H), 3.97 (t, J=6.4 Hz, 2H), 3.67-3.03 (m, 13H), 2.33-2.31 (m, 1H), 2.17-2.09 (m, 1H), 1.61-1.55 (m, 2H), 0.89 (t, J=7.2 Hz, 3H).

LCMS (ESI-TOF) m/z 494.2 [M+H⁺] with a purity of >98%.

### Propyl (3R)-4-(9-chloro-6-(3-fluoropyridin-2-yl)-5,6,7,8-tetrahydroacridine-3-carbonyl)-3-methylpiperazine-1-carboxylate (A113)

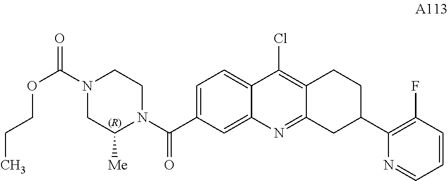

Intermediate from General Procedure F in the synthesis of A099 was subjected to General Procedure C1 with (R)-n-propyl 3-methylpiperazine-1-carboxylate as reagent, followed by General Procedure G to afford A109.

¹H NMR (400 MHz, DMSO-d₆) δ 8.39 (d, J=4.6 Hz, 1H), 8.22 (d, J=8.6 Hz, 1H), 7.95 (s, 1H), 7.72 (t, J=9.4 Hz, 1H), 7.66 (d, J=8.6 Hz, 1H), 7.39 (dt, J=8.5, 4.4 Hz, 1H), 4.81-3.58 (m, 10H), 3.22-2.83 (m, 4H), 2.29-2.18 (m, 1H), 2.18-2.02 (m, 1H), 1.66-1.44 (m, 2H), 1.17 (d, J=5.0 Hz, 3H), 0.88 (t, J=7.4 Hz, 3H).

LCMS (ESI-TOF) m/z 525.2 [M+H⁺] with a purity of >99%.

### Propyl (3R)-4-(9-chloro-6-(2-methylpyridin-4-yl)-5,6,7,8-tetrahydroacridine-3-carbonyl)-3-methylpiperazine-. 1-carboxylate (A114)

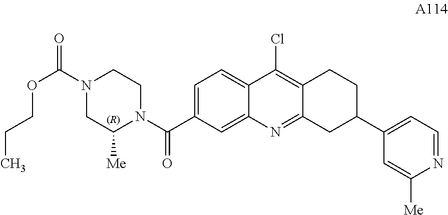

Intermediate from General Procedure F in the synthesis of A104 was subjected to General Procedure C1 with (R)-n-propyl 3-methylpiperazine-1-carboxylate as reagent, followed by General Procedure G to afford A114.

¹H NMR (400 MHz, DMSO-d₆) δ 8.39 (d, J=5.1 Hz, 1H), 8.23 (d, J=8.6 Hz, 1H), 7.95 (s, 1H), 7.66 (d, J=8.3 Hz, 1H), 7.28 (s, 1H), 7.19 (d, J=5.0 Hz, 1H), 4.92-3.58 (m, 5H), 3.36-2.85 (m, 9H), 2.47 (s, 3H), 2.29-2.14 (m, 1H), 2.14-1.93 (m, 1H), 1.67-1.47 (m, 2H), 1.17 (d, J=5.6 Hz, 3H), 0.89 (t, J=7.3 Hz, 3H). LCMS (ESI-TOF) m/z 522.0 [M+H⁺] with a purity of >98%.

### Propyl (2R)-4-(9-chloro-6-(3-fluoropyridin-2-yl)-5,6,7,8-tetrahydroacridine-3-carbonyl)-2-methylpiperazine-1-carboxylate (A115)

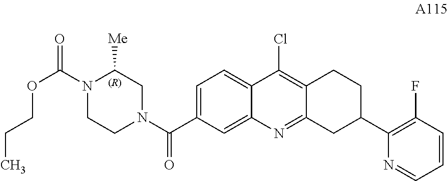

Intermediate from General Procedure F in the synthesis of A099 was subjected to General Procedure C1 with (R)-n-propyl 2-methylpiperazine-1-carboxylate as reagent, followed by General Procedure G to afford A115.

¹H NMR (400 MHz, DMSO-d₆) δ 8.39 (d, J=4.2 Hz, 1H), 8.23 (d, J=8.6 Hz, 1H), 7.95 (s, 1H), 7.75-7.63 (m, 2H), 7.39 (dt, J=8.5, 4.4 Hz, 1H), 4.60-3.55 (m, 5H), 3.51-2.82 (m, 9H), 2.29-2.18 (m, 1H), 2.17-2.04 (m, 1H), 1.66-1.49 (m, 2H), 1.33-0.95 (m, 3H), 0.88 (t, J=7.4 Hz, 3H).

LCMS (ESI-TOF) m/z 525.2 [M+H⁺] with a purity of >98%.

### Propyl 4-(9-chloro-5,6,7,8-tetrahydroacridine-3-carbonyl)-3-cyanopiperazine-1-carboxylate (A116)

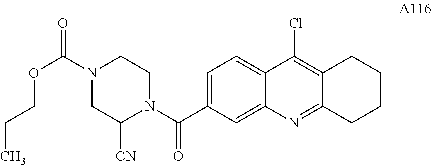 [SMILES: CCCOC(=O)N1CCN(C(=O)c2ccc3c(Cl)c4c(nc3c2)CCCC4)C(C#N)C1]

Step 1: According to General Procedure C1, commercially available 9-chloro-5,6,7,8-tetrahydroacridine-3-carboxylic acid was reacted with tert-butyl 3-cyanopiperazine-1-carboxylate to give tert-butyl 4-(9-chloro-5,6,7,8-tetrahydroacridine-3-carbonyl)-3-cyanopiperazine-1-carboxylate.

Step 2: The resulting intermediate (90 mg, 0.198 mmol) was dissolved in trifluoroacetic acid (1 mL) and dichloromethane (5 mL) for 24 h. The mixture was diluted with dichloromethane (50 mL) and the organic layer was washed with saturated sodium bicarbonate, brine, dried over anhydrous sodium sulfate, filtered and concentrated under reduced pressure to give 1-(9-chloro-5,6,7,8-tetrahydroacridine-3-carbonyl)piperazine-2-carbonitrile.

Step 3: The crude material from above (82.4 mg, 0.23 mmol) was dissolved in dichloromethane (2.0 mL) and triethylamine (64 μL, 0.46 mmol, 2 equiv) followed by n-propyl chloroformate (40 μL, 0.35 mmol, 1.5 equiv) were added at 0° C. After 1 h, the mixture was diluted with ethyl acetate (50 mL) and the organic layer was washed with saturated sodium bicarbonate, brine, dried over anhydrous sodium sulfate, and concentrated under reduced pressure. The crude material was purified by column chromatography (ethyl acetate/hexanes) to afford A116 as a white solid (44.7 mg, 44%) upon lyophilization.

¹H NMR (400 MHz, 80° C., DMSO-d₆) δ 8.23 (d, J=8.6 Hz, 1H), 8.01 (s, 1H), 7.67 (d, J=8.8 Hz, 1H), 5.53 (s, 1H), 4.28 (d, J=14.0 Hz, 1H), 4.11-3.85 (m, 4H), 3.35 (dd, J=14.1, 3.6 Hz, 1H), 3.21 (t, J=12.7 Hz, 1H), 3.15-2.97 (m, 5H), 1.91 (s, 4H), 1.73-1.42 (m, 2H), 0.91 (t, J=7.4 Hz, 3H).

LCMS (ESI-TOF) m/z 441.1 [M+H⁺] with a purity of >98%.

### Propyl 4-(9-chloro-5,6,7,8-tetrahydroacridine-3-carbonyl)-2-cyanopiperazine-1-carboxylate (A117)

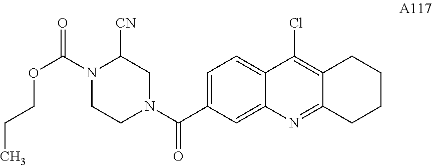 [SMILES: CCCOC(=O)N1CCN(C(=O)c2ccc3c(Cl)c4c(nc3c2)CCCC4)CC1C#N]

Step 1: According to General Procedure C1, commercially available 9-chloro-5,6,7,8-tetrahydroacridine-3-carboxylic acid was reacted with tert-butyl 2-cyanopiperazine-1-carboxylate to give tert-butyl 4-(9-chloro-5,6,7,8-tetrahydroacridine-3-carbonyl)-2-cyanopiperazine-1-carboxylate.

Step 2: The resulting intermediate (192.8 mg, 0.424 mmol) was dissolved in trifluoroacetic acid (2 mL) and dichloromethane (5 mL) for 24 h. The mixture was diluted with dichloromethane (50 mL) and the organic layer was washed with saturated sodium bicarbonate, brine, dried over anhydrous sodium sulfate, filtered and concentrated under reduced pressure to give 4-(9-chloro-5,6,7,8-tetrahydroacridine-3-carbonyl)piperazine-2-carbonitrile.

Step 3: The crude material from above (150 mg, 0.423 mmol) was dissolved in dichloromethane (2.0 mL) and triethylamine (120 μL, 0.846 mmol, 2 equiv) followed by n-propyl chloroformate (71 μL, 0.635 mmol, 1.5 equiv) were added at 0° C. After 1 h, the mixture was diluted with ethyl acetate (50 mL) and the organic layer was washed with saturated sodium bicarbonate, brine, dried over anhydrous sodium sulfate, and concentrated under reduced pressure. The crude material was purified by column chromatography (ethyl acetate/hexanes) to afford A117 as a white solid (54.3 mg, 29%) upon lyophilization.

¹H NMR (400 MHz, 80° C., DMSO-d₆) δ 8.22 (d, J=8.6 Hz, 1H), 7.95 (s, 1H), 7.64 (d, J=8.6 Hz, 1H), 5.28 (s, 1H), 4.46-3.81 (m, 5H), 3.42 (d, J=13.3 Hz, 1H), 3.25-2.87 (m, 6H), 1.91 (s, 4H), 1.69-1.54 (m, 2H), 0.91 (t, J=7.4 Hz, 3H).

LCMS (ESI-TOF) m/z 441.1 [M+H⁺] with a purity of >97%.

### Propyl 4-(9-chloro-5-methyl-5,6,7,8-tetrahydroacridine-3-carbonyl)piperazine-1-carboxylate (A118)

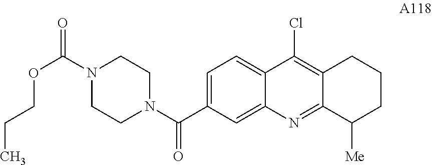 [SMILES: CCCOC(=O)N1CCN(C(=O)c2ccc3c(Cl)c4c(nc3c2)C(C)CCC4)CC1]

Step 1: According to General Procedure I, 2-amino-terephthalic acid was cyclized with 2-methylcyclohexanone (2 equiv) to obtain crude 5-methyl-9-oxo-5,6,7,8,9,10-hexahydroacridine-3-carboxylic acid.

Step 2: The resulting intermediate was reacted with n-propyl piperazine-1-carboxylate via General Procedure C1 to obtain propyl 4-(5-methyl-9-oxo-5,6,7,8,9,10-hexahydroacridine-3-carbonyl)piperazine-1-carboxylate.

Step 3: The material from above was subjected to General Procedure B to afford A118 as a white solid upon lyophilization.

¹H NMR (400 MHz, DMSO-d₆) δ 8.19 (d, J=8.6 Hz, 1H), 7.97 (d, J=1.2 Hz, 1H), 7.66 (dd, J=8.6, 1.6 Hz, 1H), 3.97 (t, J=6.6 Hz, 2H), 3.79-3.34 (m, 8H), 3.18-3.07 (m, 1H), 3.00 (t, J=6.5 Hz, 2H), 2.13-1.91 (m, 2H), 1.83 (ddd, J=10.0, 9.6, 4.9 Hz, 1H), 1.73-1.52 (m, 3H), 1.43 (d, J=7.0 Hz, 3H), 0.89 (t, J=7.3 Hz, 3H).

LCMS (ESI-TOF) m/z 430.2 [M+H⁺] with a purity of >99%.

### Propyl 4-(9-chloro-5-phenyl-5,6,7,8-tetrahydroacridine-3-carbonyl)piperazine-1-carboxylate (A119)

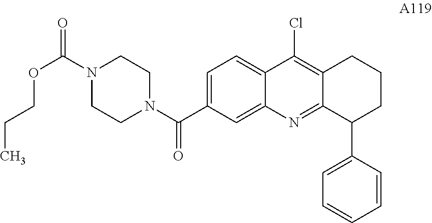 [SMILES: CCCOC(=O)N1CCN(C(=O)c2ccc3c(Cl)c4c(nc3c2)C(c2ccccc2)CCC4)CC1]

Compound A119 was synthesized using General Procedure I with 2-amino-terephthalic acid and 2-phenylcyclohexanone, followed by General Procedure C1 using n-propyl piperazine-1-carboxylate as reagent.

¹H NMR (400 MHz, DMSO-d₆) δ 8.24 (d, J=8.6 Hz, 1H), 7.84 (d, J=1.2 Hz, 1H), 7.67 (dd, J=8.6, 1.5 Hz, 1H), 7.26 (t, J=7.4 Hz, 2H), 7.17 (t, J=7.3 Hz, 1H), 7.01 (d, J=7.2 Hz, 2H), 4.52 (t, J=5.6 Hz, 1H), 3.96 (t, J=6.6 Hz, 2H), 3.80-3.34 (m, 8H), 3.21-2.96 (m, 2H), 2.23 (td, J=12.8, 6.2 Hz, 1H), 2.04 (td, J=10.7, 5.3 Hz, 1H), 1.92-1.77 (m, 2H), 1.65-1.48 (m, 2H), 0.87 (t, J=7.3 Hz, 3H).

LCMS (ESI-TOF) m/z 492.2 [M+H⁺] with a purity of >99%.

### Propyl (3R)-4-(9-chloro-6-(2-(dimethylamino)ethyl)-5,6,7,8-tetrahydroacridine-3-carbonyl)-3-methylpiperazine-1-carboxylate (A120)

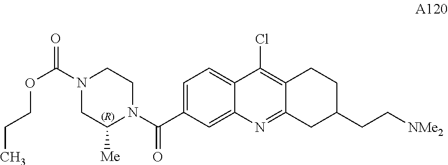

Compound A120 was prepared according to General Procedure A, B and C2 using 3-(2-(dimethylamino)ethyl)cyclohexanone (General Procedure A) and (R)-n-propyl 3-methylpiperazine-1-carboxylate (General Procedure C2) as starting materials.

¹H NMR (400 MHz, DMSO-d₆) δ 8.19 (d, J=8.4 Hz, 1H), 7.91 (s, 1H), 7.64 (d, J=9.2 Hz, 1H), 4.25-3.69 (m, 4H), 3.26-2.84 (m, 7H), 2.76-2.69 (m, 1H), 2.39-2.32 (m, 3H), 2.14 (s, 6H), 2.07-2.04 (m, 1H), 1.98-1.93 (m, 1H), 1.60-1.47 (m, 5H), 1.16-1.15 (m, 3H), 0.88 (t, J=7.2 Hz, 3H).

LCMS (ESI-TOF) m/z 501.6 [M+H⁺] with a purity of >97%.

### Propyl 4-(9-chloro-6-(2-(pyrrolidin-1-yl)ethyl)-5,6,7,8-tetrahydroacridine-3-carbonyl)piperazine-1-carboxylate (A121)

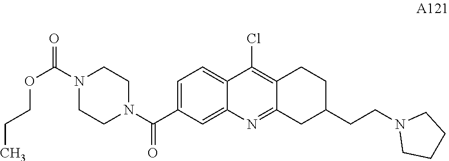 [SMILES: CCCOC(=O)N1CCN(C(=O)c2ccc3c(Cl)c4c(nc3c2)CC(CCN2CCCC2)CC4)CC1]

Compound A121 was prepared according to General Procedure A, B and C2 using 3-(2-(pyrrolidine)ethyl)cyclohexanone (General Procedure A) and n-propyl piperazine-1-carboxylate (General Procedure C2) as starting materials.

¹H NMR (400 MHz, DMSO-d₆) δ 8.19 (d, J=8.8 Hz, 1H), 7.94 (s, 1H), 7.65 (d, J=8.0 Hz, 1H), 3.97 (t, J=6.4 Hz, 2H), 3.66-3.36 (m, 8H), 3.21-3.10 (m, 4H), 2.92-2.86 (m, 3H), 2.77-2.67 (m, 3H), 2.05-1.96 (m, 2H), 1.70-1.55 (m, 9H), 0.88 (t, J=7.6 Hz, 3H).

LCMS (ESI-TOF) m/z 513.5 [M+H⁺] with a purity of >95%.

### Propyl (2R)-4-(9-chloro-6-(2-methylpyridin-4-yl)-5,6,7,8-tetrahydroacridine-3-carbonyl)-2-methylpiperazine-1-carboxylate (A122)

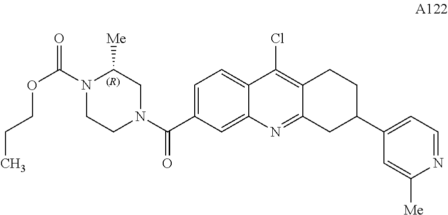

Intermediate from General Procedure F in the synthesis of A104 was subjected to General Procedure C1 with (R)-n-propyl 2-methylpiperazine-1-carboxylate as reagent, followed by General Procedure G to afford A122.

¹H NMR (400 MHz, DMSO-d₆) δ 8.39 (d, J=5.1 Hz, 1H), 8.24 (d, J=8.6 Hz, 1H), 7.96 (br s, 1H), 7.69 (br s, 1H), 7.28 (s, 1H), 7.19 (d, J=5.8 Hz, 1H), 4.58-4.04 (m, 2H), 4.03-3.91 (m, 2H), 3.91-3.34 (m, 4H), 3.27-2.89 (m, 6H), 2.47 (s, 3H), 2.21 (br s, 1H), 2.12-1.95 (m, 1H), 1.65-1.51 (m, 2H), 1.29-0.93 (m, 3H), 0.88 (t, J=7.4 Hz, 3H).

LCMS (ESI-TOF) m/z 521.2 [M+H⁺] with a purity of >99%.

### Propyl (2S)-4-(9-chloro-6-(2-methylpyridin-4-yl)-5,6,7,8-tetrahydroacridine-3-carbonyl)-2-methylpiperazine-1-carboxylate (A123)

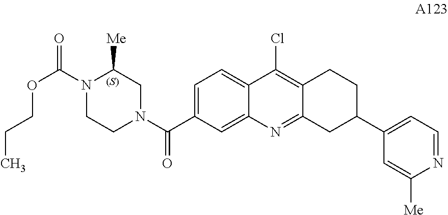

Intermediate from General Procedure F in the synthesis of A104 was subjected to General Procedure C1 with (S)-n-propyl 2-methylpiperazine-1-carboxylate as reagent, followed by General Procedure G to afford A123.

¹H NMR (400 MHz, DMSO-d₆) δ 8.39 (d, J=5.1 Hz, 1H), 8.24 (d, J=8.6 Hz, 1H), 7.95 (br s, 1H), 7.68 (br s, 1H), 7.28 (s, 1H), 7.20 (d, J=4.8 Hz, 1H), 4.60-4.03 (m, 2H), 4.05-3.92 (m, 2H), 3.90-3.43 (m, 4H), 3.28-2.87 (m, 6H), 2.47 (s, 3H), 2.21 (br s, 1H), 2.12-1.95 (m, 1H), 1.65-1.47 (m, 2H), 1.30-0.94 (m, 3H), 0.88 (t, J=7.4 Hz, 3H).

LCMS (ESI-TOF) m/z 521.2 [M+H⁺] with a purity of >98%.

### Propyl (2S)-4-(9-chloro-6-(3-fluoropyridin-2-yl)-5,6,7,8-tetrahydroacridine-3-carbonyl)-2-methylpiperazine-1-carboxylate (A124)

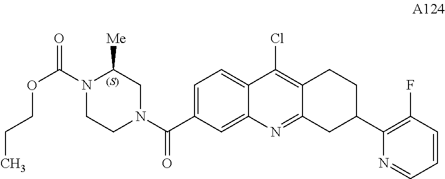

Intermediate from General Procedure F in the synthesis of A099 was subjected to General Procedure C1 with (S)-n-propyl 2-methylpiperazine-1-carboxylate as reagent, followed by General Procedure G to afford A124.

¹H NMR (400 MHz, DMSO-d₆) δ 8.39 (dd, J=3.2, 1.4 Hz, 1H), 8.23 (d, J=8.6 Hz, 1H), 7.95 (br s, 1H), 7.78-7.58 (m, 2H), 7.39 (dt, J=8.5, 4.4 Hz, 1H), 4.55-4.06 (m, 2H), 4.05-3.90 (m, 2H), 3.90-3.34 (m, 6H), 3.23-2.88 (m, 4H), 2.29-2.18 (m, 1H), 2.11 (ddd, J=16.4, 13.2, 8.3 Hz, 1H), 1.64-1.51 (m, 2H), 1.22-0.98 (m, 3H), 0.88 (t, J=7.4 Hz, 3H).

LCMS (ESI-TOF) m/z 525.2 [M+H⁺] with a purity of >98%.

### Propyl 4-(9-chloro-5,6,7,8-tetrahydroacridine-3-carbonyl)-3-(hydroxymethyl)piperazine-1-carboxylate (A125)

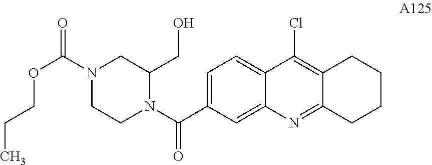 [SMILES: CCCOC(=O)N1CCN(C(=O)c2ccc3c(Cl)c4c(nc3c2)CCCC4)C(CO)C1]

Step 1: According to General Procedure C1, commercially available 9-chloro-5,6,7,8-tetrahydroacridine-3-carboxylic acid was reacted with tert-butyl 3-(hydroxymethyl)piperazine-1-carboxylate to give tert-butyl 4-(9-chloro-5,6,7,8-tetrahydroacridine-3-carbonyl)-3-(hydroxymethyl)piperazine-1-carboxylate.

Step 2: The resulting intermediate (230 mg, 0.5 mmol) was dissolved in trifluoroacetic acid (2 mL) and dichloromethane (5 mL) for 2 h. The mixture was diluted with dichloromethane (50 mL) and the organic layer was washed with saturated sodium bicarbonate, brine, dried over anhydrous sodium sulfate, filtered and concentrated under reduced pressure to give (9-chloro-5,6,7,8-tetrahydroacridin-3-yl)(2-(hydroxymethyl)piperazin-1-yl)methanone.

Step 3: The crude material from above (180 mg, 0.5 mmol) was dissolved in dichloromethane (5.0 mL) and triethylamine (0.14 mL, 1.0 mmol, 2 equiv) followed by n-propyl chloroformate (57 μL, 0.5 mmol, 1 equiv) were added at 0° C. After 1 h, the mixture was diluted with ethyl acetate (50 mL) and the organic layer was washed with saturated sodium bicarbonate, brine, dried over anhydrous sodium sulfate, and concentrated under reduced pressure. The crude material was purified by preparative-HPLC to afford A125 as a white solid (60 mg, 27%) upon lyophilization.

¹H NMR (400 MHz, 80° C., DMSO-d₆) δ 8.16 (d, J=8.6 Hz, 1H), 7.95 (s, 1H), 7.63 (d, J=8.7 Hz, 1H), 4.66 (s, 1H), 4.38-3.69 (m, 6H), 3.63-3.39 (m, 2H), 3.20-2.89 (m, 6H), 1.90 (s, 4H), 1.63-1.49 (m, 2H), 0.89 (t, J=7.4 Hz, 3H).

LCMS (ESI-TOF) m/z 446.1 [M+H⁺] with a purity of >97%.

### Propyl (3S)-4-(9-chloro-6-(2-(dimethylamino)ethyl)-5,6,7,8-tetrahydroacridine-3-carbonyl)-3-methylpiperazine-1-carboxylate (A126)

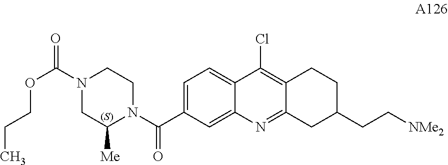

Compound A126 was prepared according to General Procedure A, B and C2 using 3-(2-(dimethylamino)ethyl)cyclohexanone (General Procedure A) and (S)-n-propyl 3-methylpiperazine-1-carboxylate (General Procedure C2) as starting materials.

¹H NMR (400 MHz, DMSO-d₆) δ 8.19 (d, J=8.4 Hz, 1H), 7.90 (s, 1H), 7.64 (d, J=8.8 Hz, 1H), 4.00-3.78 (m, 5H), 3.26-3.10 (m, 3H), 2.92-2.86 (m, 3H), 2.76-2.66 (m, 2H), 2.35-2.33 (m, 2H), 2.14 (s, 6H), 2.10-2.04 (m, 1H), 1.99-1.93 (m, 1H), 1.60-1.50 (m, 5H), 1.21-1.15 (m, 3H) 0.88 (t, J=7.2 Hz, 3H).

LCMS (ESI-TOF) m/z 501.6 [M+H⁺] with a purity of >98%.

### Propyl (2R)-4-(9-chloro-5-methyl-5,6,7,8-tetrahydroacridine-3-carbonyl)-2-methylpiperazine-1-carboxylate (A127)

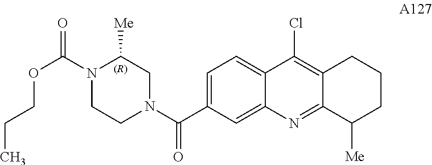

Compound A127 was synthesized using General Procedure I with 2-amino-terephthalic acid and 2-methylcyclohexanone, followed by General Procedure C1 using (R)-n-propyl 2-methylpiperazine-1-carboxylate as reagent.

¹H NMR (400 MHz, 80° C., DMSO-d₆) δ 8.18 (d, J=8.5 Hz, 1H), 7.93 (s, 1H), 7.62 (d, J=8.1 Hz, 1H), 4.41-3.63 (m, 6H), 3.36-2.95 (m, 6H), 2.14-2.03 (m, 1H), 2.02-1.90 (m, 1H), 1.90-1.77 (m, 1H), 1.71-1.52 (m, 3H), 1.43 (d, J=7.0 Hz, 3H), 1.11 (d, J=6.3 Hz, 3H), 0.89 (t, J=7.4 Hz, 3H).

LCMS (ESI-TOF) m/z 444.1 [M+H⁺] with a purity of >98%.

### Propyl 4-(9-chloro-6-(thiazol-2-yl)-5,6,7,8-tetrahydroacridine-3-carbonyl)piperazine-1-carboxylate (A128)

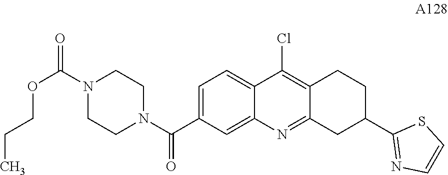 [SMILES: CCCOC(=O)N1CCN(C(=O)c2ccc3c(Cl)c4c(nc3c2)CC(c2nccs2)CC4)CC1]

Compound A128 was prepared according to General Procedure A, B and C1 using 3 3-(thiazol-2-yl)cyclohexan-1-one (General Procedure A) and n-propyl piperazine-1-carboxylate (General Procedure C1) as starting materials.

¹H NMR (400 MHz, DMSO-d₆) δ 8.22 (d, J=8.6 Hz, 1H), 7.99 (d, J=1.2 Hz, 1H), 7.75 (d, J=3.3 Hz, 1H), 7.68 (dd, J=8.6, 1.5 Hz, 1H), 7.66 (d, J=3.3 Hz, 1H), 3.98 (t, J=6.6 Hz, 2H), 3.84-3.34 (m, 11H), 3.11 (t, J=7.6 Hz, 2H), 2.49-2.40 (m, 1H), 2.22-2.07 (m, 1H), 1.64-1.54 (m, 2H), 0.89 (t, J=7.3 Hz, 3H).

LCMS (ESI-TOF) m/z 499.1 [M+H⁺] with a purity of >98%.

### Propyl (2R)-4-(9-chloro-6-(thiazol-2-yl)-5,6,7,8-tetrahydroacridine-3-carbonyl)-2-methylpiperazine-1-carboxylate (A129)

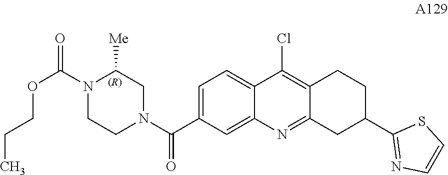

Compound A129 was prepared according to General Procedure A, B and C1 using 3 3-(thiazol-2-yl)cyclohexan-1-one (General Procedure A) and (R)-n-propyl 2-methylpiperazine-1-carboxylate (General Procedure C1) as starting materials.

¹H NMR (400 MHz, DMSO-d₆) δ 8.22 (d, J=8.6 Hz, 1H), 7.97 (br s, 1H), 7.75 (dd, J=3.3, 1.0 Hz, 1H), 7.72-7.61 (m, 2H), 4.54-4.03 (m, 2H), 4.03-3.89 (m, 2H), 3.89-3.65 (m, 2H), 3.63-3.26 (m, 4H), 3.24-2.86 (m, 4H), 2.48-2.39 (m, 1H), 2.24-2.08 (m, 1H), 1.67-1.51 (m, 2H), 1.29-0.96 (m, 3H), 0.89 (t, J=7.4 Hz, 3H).

LCMS (ESI-TOF) m/z 513.1 [M+H⁺] with a purity of >97%.

### Propyl 4-(9-chloro-6-(5-methylpyridin-2-yl)-5,6,7,8-tetrahydroacridine-3-carbonyl)piperazine-1-carboxylate (A130)

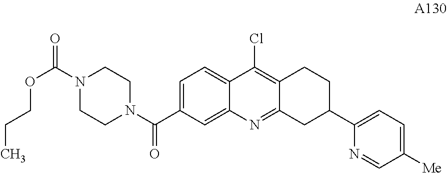 [SMILES: CCCOC(=O)N1CCN(C(=O)c2ccc3c(Cl)c4c(nc3c2)CC(c2ccc(C)cn2)CC4)CC1]

Compound A130 was prepared according to General Procedure E, F, C1 and G using 3-(5-methylpyridin-2-yl)cyclohexan-1-one (General Procedure E) and n-propyl piperazine-1-carboxylate (General Procedure C1) as starting materials.

¹H NMR (400 MHz, 80° C., DMSO-d₆) δ 8.36 (s, 1H), 8.20 (d, J=8.5 Hz, 1H), 7.95 (s, 1H), 7.65 (d, J=8.3 Hz, 1H), 7.56 (d, J=7.9 Hz, 1H), 7.28 (d, J=8.0 Hz, 1H), 3.99 (t, J=6.5 Hz, 2H), 3.75-3.11 (m, 13H), 2.28 (s, 4H), 2.08 (br s, 1H), 1.59 (dd, J=14.2, 7.3 Hz, 2H), 0.89 (t, J=7.4 Hz, 3H).

LCMS (ESI-TOF) m/z 507.2 [M+H⁺] with a purity of >98%.

### Propyl (2R)-4-(9-chloro-6-(5-methylpyridin-2-yl)-5,6,7,8-tetrahydroacridine-3-carbonyl)-2-methylpiperazine-1-carboxylate (A131)

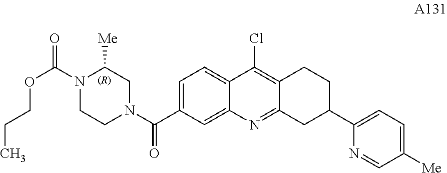

Compound A131 was prepared according to General Procedure E, F, C1 and G using 3-(5-methylpyridin-2-yl)cyclohexan-1-one (General Procedure E) and (R)-n-propyl 2-methylpiperazine-1-carboxylate (General Procedure C1) as starting materials.

¹H NMR (400 MHz, 80° C., DMSO-d₆) δ 8.35 (s, 1H), 8.21 (d, J=8.6 Hz, 1H), 7.93 (s, 1H), 7.64 (d, J=8.6 Hz, 1H), 7.56 (d, J=7.9 Hz, 1H), 7.28 (d, J=7.8 Hz, 1H), 4.56-3.67 (m, 6H), 3.45-3.10 (m, 8H), 2.28 (s, 4H), 2.16-2.01 (m, 1H), 1.73-1.47 (m, 2H), 1.11 (d, J=6.3 Hz, 3H), 0.89 (t, J=7.4 Hz, 3H).

LCMS (ESI-TOF) m/z 521.2 [M+H⁺] with a purity of >97%.

### Propyl 4-(9-chloro-6-(4-methylpyridin-2-yl)-5,6,7,8-tetrahydroacridine-3-carbonyl)piperazine-1-carboxylate (A132)

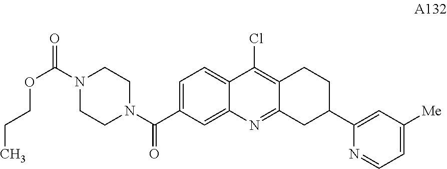 [SMILES: CCCOC(=O)N1CCN(C(=O)c2ccc3c(Cl)c4c(nc3c2)CC(c2cc(C)ccn2)CC4)CC1]

Compound A132 was prepared according to General Procedure E, F, C1 and G using 3-(4-methylpyridin-2-yl)cyclohexanone (General Procedure E) and n-propyl piperazine-1-carboxylate (General Procedure C1) as starting materials.

¹H NMR (400 MHz, 80° C., DMSO-d₆) δ 8.36 (d, J=5.1 Hz, 1H), 8.20 (d, J=8.6 Hz, 1H), 7.95 (s, 1H), 7.64 (d, J=8.6 Hz, 1H), 7.23 (s, 1H), 7.06 (d, J=4.5 Hz, 1H), 3.99 (t, J=6.6 Hz, 2H), 3.69-3.26 (m, 11H), 3.17 (dd, J=14.1, 8.4 Hz, 2H), 2.32 (s, 3H), 2.27 (br s, 1H), 2.15-2.02 (m, 1H), 1.66-1.51 (m, 2H), 0.89 (t, J=7.4 Hz, 3H).

LCMS (ESI-TOF) m/z 507.2 [M+H⁺] with a purity of >99%.

### Propyl (2R)-4-(9-chloro-6-(4-methylpyridin-2-yl)-5,6,7,8-tetrahydroacridine-3-carbonyl)-2-methylpiperazine-1-carboxylate (A133)

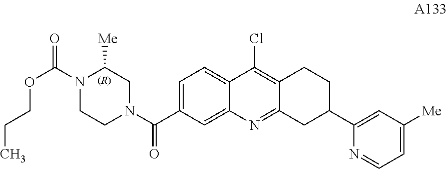

Compound A133 was prepared according to General Procedure E, F, C1 and G using 3-(4-methylpyridin-2-yl)cyclohexanone (General Procedure E) and (R)-n-propyl 2-methylpiperazine-1-carboxylate (General Procedure C1) as starting materials.

¹H NMR (400 MHz, 80° C., DMSO-d₆) δ 8.36 (d, J=5.0 Hz, 1H), 8.21 (d, J=8.7 Hz, 1H), 7.93 (s, 1H), 7.64 (d, J=8.3 Hz, 1H), 7.23 (s, 1H), 7.06 (d, J=4.5 Hz, 1H), 4.39-3.65 (m, 6H), 3.44-3.06 (m, 8H), 2.32 (s, 3H), 2.27 (br s, 1H), 2.17-2.00 (m, 1H), 1.64-1.52 (m, 2H), 1.11 (d, J=6.6 Hz, 3H), 0.89 (t, J=7.4 Hz, 3H).

LCMS (ESI-TOF) iniz 521.2 [M+H⁺] with a purity of >99%.

### Propyl 4-(9-chloro-6-(pyrimidin-4-yl)-5,6,7,8-tetrahydroacridine-3-carbonyl)piperazine-1-carboxylate (A134)

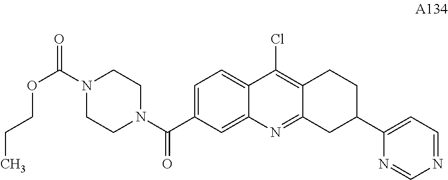 [SMILES: CCCOC(=O)N1CCN(C(=O)c2ccc3c(Cl)c4c(nc3c2)CC(c2ccncn2)CC4)CC1]

Compound A134 was prepared according to General Procedure E, F, C2 and G using 3-(pyrimidin-4-yl)cyclohexanone (General Procedure E) and n-propyl piperazine-1-carboxylate (General Procedure C2) as starting materials.

¹H NMR (400 MHz, CDCl₃) δ 9.19 (s, 1H), 8.68 (d, J=5.2 Hz, 1H), 8.27 (d, J=8.8 Hz, 1H), 8.00(s, 1H), 7.62 (dd, J=1.6, 8.4 Hz, 1H), 7.28 (d, J=6.4 Hz, 1H), 4.08 (t, J=6.4 Hz, 2H), 3.81-3.36 (m, 10H), 3.34-3.27 (m, 2H), 3.12-3.03 (m, 1H), 2.44-2.41 (m, 1H), 2.22-2.16 (m, 1H), 1.69-1.64 (m, 2H), 0.95 (t, J=7.6 Hz, 3H).

LCMS (ESI-TOF) m/z 494.3 [M+H⁺] with a purity of >99%.

### Propyl (2R)-4-(9-chloro-6-(2-(dimethylamino)ethyl)-5,6,7,8-tetrahydroacridine-3-carbonyl)-2-methylpiperazine-1-carboxylate (A135)

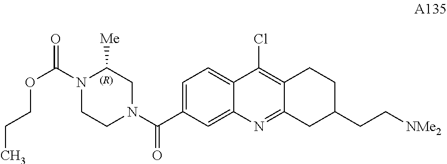

Compound A135 was prepared according to General Procedure A, B and C2 using 3-(2-(dimethylamino)ethyl)cyclohexanone (General Procedure A) and (R)-n-propyl 2-methylpiperazine-1-carboxylate (General Procedure C2) as starting materials.

¹H NMR (400 MHz, DMSO-d₆) δ 8.19 (d, J=8.8 Hz, 1H), 7.95-7.88 (m, 1H), 7.71-7.61 (m, 1H), 4.50-3.90 (m, 4H), 3.89-3.39 (m, 2H), 3.21-2.85 (m, 6H), 2.76-2.72 (m, 1H), 2.37-2.31 (m, 2H), 2.19 (s, 6H), 2.21-1.93 (m, 2H), 1.60-1.49 (m, 5H), 1.17-1.00 (m, 3H), 0.88 (t, J=7.2 Hz, 3H).

LCMS (ESI-TOF) m/z 501.4 [M+H⁺] with a purity of >97%.

### Propyl (2S)-4-(9-chloro-6-(2-(dimethylamino)ethyl)-5,6,7,8-tetrahydroacridine-3-carbonyl)-2-methylpiperazine-1-carboxylate (A136)

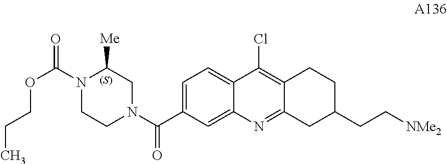

Compound A136 was prepared according to General Procedure A, B and C2 using 3-(2-(dimethylamino)ethyl)cyclohexanone (General Procedure A) and (S)-n-propyl 2-methylpiperazine-1-carboxylate (General Procedure C2) as starting materials.

¹H NMR (400 MHz, DMSO-d₆) δ 8.19 (d, J=8.8 Hz, 1H), 7.92 (br s, 1H), 7.71-7.60 (m, 1H), 4.50-3.87 (m, 4H), 3.72-3.35 (m, 2H), 3.25-2.84 (m, 6H), 2.76-2.67 (m, 1H), 2.39-2.28 (m, 2H), 2.19 (s, 6H), 2.20-1.93 (m, 2H), 1.62-1.48 (m, 5H), 1.17-1.00 (m, 3H), 0.88 (t, J=7.2 Hz, 3H).

LCMS (ESI-TOF) m/z 501.4 [M+H⁺] with a purity of >98%.

### Propyl (3S)-4-(9-chloro-6-(prop-2-yn-1-yl)-5,6,7,8-tetrahydroacridine-3-carbonyl)-3-methylpiperazine-1-carboxylate (A137)

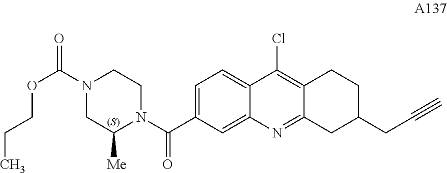

Compound A137 was prepared according to General Procedure A, B and C2 using 3-(prop-2-ynyl)cyclohexanone (General Procedure A) and (S)-n-propyl 3-methylpiperazine-1-carboxylate (General Procedure C2) as starting materials.

¹H NMR (400 MHz, DMSO-d₆) δ 8.20 (d, J=8.8 Hz, 1H), 7.92 (s, 1H), 7.64 (d, J=7.2 Hz,1H), 4.61-3.65 (m, 7H), 3.24-3.14 (m, 4H), 2.95-2.80 (m, 4H), 2.36-2.32 (m, 1H), 2.22-2.11 (m, 2H), 1.63-1.55 (m, 3H), 1.16 (s, 3H), 0.89 (t, J=7.2 Hz, 3H).

LCMS (ESI-TOF) m/z 486.3 [M+H⁺] with a purity of >99%.

### Propyl 4-(9-chloro-6-(2-cyanopyridin-4-yl)-5,6,7,8-tetrahydroacridine-3-carbonyl)piperazine-1-carboxylate (A138)

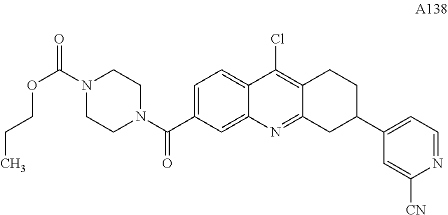 [SMILES: CCCOC(=O)N1CCN(C(=O)c2ccc3c(Cl)c4c(nc3c2)CC(c2ccnc(C#N)c2)CC4)CC1]

Compound A138 was prepared according to General Procedure E, F, C2 and G using 4-(3-oxocyclohexyl)picolinonitrile (General Procedure E) and n-propyl piperazine-1-carboxylate (General Procedure C2) as starting materials.

¹H NMR (400 MHz, DMSO-d₆) δ 8.72 (d, J=4.8 Hz, 1H), 8.23 (d, J=8.4 Hz, 1H), 8.13 (s, 2H), 7.98 (s, 1H), 7.77 (dd, J=4.8 Hz, 1.6 Hz, 1H), 7.69 (d, J=8.4 Hz, 1H), 3.98 (t, J=6.4 Hz, 2H), 3.70-3.31 (m, 10H), 3.24-3.21 (m, 2H), 3.09-3.04 (m, 1H), 2.31-2.21 (m, 1H), 2.11-2.08 (m, 1H), 1.60-1.55 (m, 2H), 0.89 (t, J=7.2 Hz, 3H).

LCMS (ESI-TOF) m/z 518.3 [M+H⁺] with a purity of >97%.

### Propyl 4-(6-(2-carbamoylpyridin-4-yl)-9-chloro-5,6,7,8-tetrahydroacridine-3-carbonyl)piperazine-1-carboxylate (A139)

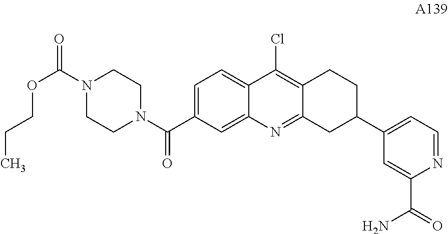 [SMILES: CCCOC(=O)N1CCN(C(=O)c2ccc3c(Cl)c4c(nc3c2)CC(c2ccnc(C(N)=O)c2)CC4)CC1]

Compound A138 was dissolved in dimethylsulfoxide and was cooled to 0° C. Potassium carbonate (5 equiv) followed by 30% hydrogen peroxide (2 equiv) was added and the reaction mixture was stirred at room temperature for 1 h. The reaction was diluted with ethyl acetate and the organic layer was washed with water, brine, dried over anhydrous sodium sulfate, filtered and concentrated to afford compound A139.

¹H NMR (400 MHz, DMSO-d₆) δ 8.60 (d, J=4.8 Hz, 1H), 8.23 (d, J=8.4 Hz, 1H), 8.11 (s, 1H), 8.06 (s, 1H), 7.98 (s, 1H), 7.69-7.61 (m, 3H), 3.98 (t, J=6.4 Hz, 2H), 3.70-3.31 (m, 10H), 3.25-3.07 (m, 3H), 2.31-2.25 (m, 1H), 2.41-1.91 (m, 1H), 1.60-1.55 (m, 2H), 0.89 (t, J=7.2 Hz, 3H).

LCMS (ESI-TOF) m/z 536.4 [M+H⁺] with a purity of >97%.

### Propyl (2R)-4-(9-chloro-6-(2-cyanopyridin-4-yl)-5,6,7,8-tetrahydroacridine-3-carbonyl)-2-methylpiperazine-1-carboxylate (A140)

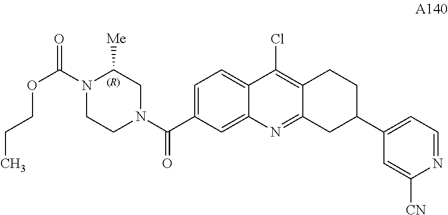

Compound A140 was prepared according to General Procedure E, F, C2 and G using 4-(3-oxocyclohexyl)picolinonitrile (General Procedure E) and (R)-n-propyl 2-methylpiperazine-1-carboxylate (General Procedure C2) as starting materials.

¹H NMR (400 MHz, DMSO-d₆) δ 8.72 (d, J=5.2 Hz, 1H), 8.24 (d, J=8.8 Hz, 1H), 8.13 (s, 1H), 7.96 (br s, 1H), 7.77 (d, J=4.4 Hz, 1H), 7.69 (br s, 1H), 4.51-3.41 (m, 9H), 3.25-3.01 (m, 5H), 2.31-2.21 (m, 1H), 2.19-2.01 (m, 1H), 1.62-1.53 (m, 2H), 1.23-1.00 (m, 3H), 0.89 (t, J=7.4 Hz, 3H).

LCMS (ESI-TOF) m/z 532.5 [M+H⁺] with a purity of >95%.

### Propyl (2R)-4-(6-(2-carbamoylpyridin-4-yl)-9-chloro-5,6,7,8-tetrahydroacridine-3-carbonyl)-2-methylpiperazine-1-carboxylate (A141)

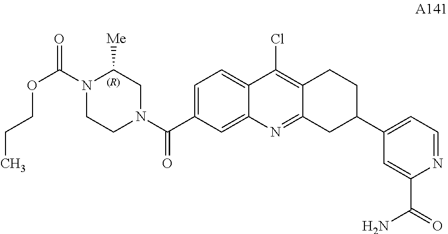

Compound A140 was dissolved in dimethylsulfoxide and was cooled to 0° C. Potassium carbonate (5 equiv) followed by 30% hydrogen peroxide (2 equiv) was added and the reaction mixture was stirred at room temperature for 1 h. The reaction was diluted with ethyl acetate and the organic layer was washed with water, brine, dried over anhydrous sodium sulfate, filtered and concentrated to afford compound A141.

¹H NMR (400 MHz, DMSO-d₆) δ 8.61 (d, J=4.8 Hz, 1H), 8.27 (d, J=9.2 Hz, 1H), 8.06 (s, 1H), 7.96 (s, 1H), 7.91-7.72 (s, 2H), 7.69-7.63 (m, 2H), 4.49-3.77 (m, 5H), 3.51-3.08 (m, 9H), 2.40-2.26 (m, 1H), 2.22-2.02 (m, 1H), 1.61-1.55 (m, 2H), 1.25-0.90 (m, 3H), 0.89 (t, J=7.2 Hz, 3H).

LCMS (ESI-TOF) m/z 550.3 [M+H⁺] with a purity of >96%.

### Propyl (2R)-4-(9-chloro-6-(prop-2-yn-1-yl)-5,6,7,8-tetrahydroacridine-3-carbonyl)-2-methylpiperazine-1-carboxylate (A142)

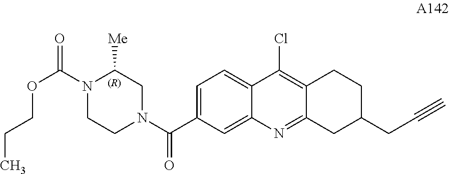

Compound A142 was prepared according to General Procedure A, B and C2 using 3-(prop-2-ynyl)cyclohexanone (General Procedure A) and (R)-n-propyl 2-methylpiperazine-1-carboxylate (General Procedure C2) as starting materials.

¹H NMR (400 MHz, DMSO-d₆) δ 8.20 (d, J=8.8 Hz, 1H), 7.93 (br s, 1H), 7.66 (br s, 1H), 4.51-3.45 (m, 6H), 3.25-2.79 (m, 8H), 2.36-2.34 (m, 2H), 2.22-2.12 (m, 2H), 1.63-1.53 (m, 3H), 1.31-1.16 (br s, 3H), 0.89 (t, J=7.2 Hz, 3H).

LCMS (ESI-TOF) m/z 468.2 [M+H⁺] with a purity of >98%.

### Propyl (3S)-4-(9-chloro-6-(2-cyanopyridin-4-yl)-5,6,7,8-tetrahydroacridine-3-carbonyl)-3-methylpiperazine-1-carboxylate (A143)

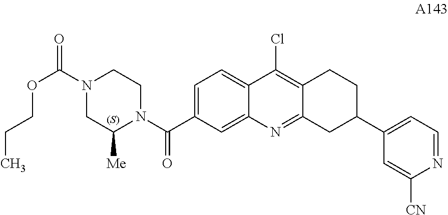

Compound A143 was prepared according to General Procedure E, F, C2 and G using 4-(3-oxocyclohexyl)picolinonitrile (General Procedure E) and (S)-n-propyl 3-methylpiperazine-1-carboxylate (General Procedure C2) as starting materials.

¹H NMR (400 MHz, DMSO-d₆) δ 8.73 (d, J=5.6 Hz, 1H), 8.24 (d, J=8.8 Hz, 1H), 8.14 (s, 1H), 7.95 (s, 1H), 7.77 (d, J=4.0 Hz, 1H), 7.67 (d, J=9.6 Hz, 1H), 4.01-3.71 (m, 3H), 3.41-3.27 (m, 4H), 3.21-2.91 (m, 7H), 2.32-2.20 (m, 1H), 2.15-2.09 (m, 1H),1.60-1.55 (m, 2H), 1.23-1.10 (m, 3H), 0.89 (t, J=7.4 Hz, 3H).

LCMS (ESI-TOF) m/z 532.2 [M+H⁺] with a purity of >98%.

### Propyl 4-(6-(2-(aminomethyl)pyridin-4-yl)-9-chloro-5,6,7,8-tetrahydroacridine-3-carbonyl)piperazine-1-carboxylate (A144)

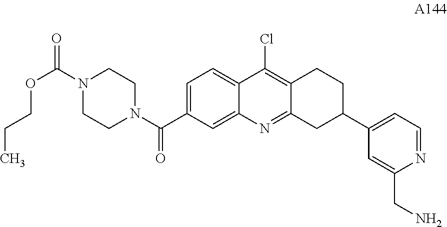 [SMILES: CCCOC(=O)N1CCN(C(=O)c2ccc3c(Cl)c4c(nc3c2)CC(c2ccnc(CN)c2)CC4)CC1]

Compound A144 was synthesized according to General Procedure D using compound A138 as starting material.

¹H NMR (400 MHz, DMSO-d₆) δ 8.57 (d, J=5.6 Hz, 1H), 8.26 (br s, 2H), 8.21 (d, J=8.8 Hz, 1H), 7.95 (s, 1H), 7.67 (dd, J=8.8 Hz, 1.6 Hz, 1H), 7.51 (s, 1H), 7.42 (d, J=4.0 Hz, 1H), 4.18 (t, J=5.6 Hz, 2H), 3.96 (t, J=6.4 Hz, 2H), 3.70-3.31 (m, 6H), 3.29-3.09 (m, 5H), 3.09-3.06 (m, 2H), 2.25-2.21 (m, 1H), 2.10-2.04 (m, 1H), 1.58-1.53 (m, 2H), 0.89 (t, J=7.4 Hz, 3H).

LCMS (ESI-TOF) m/z 522.3 [M+H⁺] with a purity of >95%.

### Propyl (3S)-4-(6-(2-carbamoylpyridin-4-yl)-9-chloro-5,6,7,8-tetrahydroacridine-3-carbonyl)-3-methylpiperazine-1-carboxylate (A145)

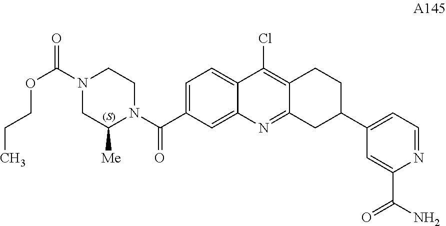

Compound A143 was dissolved in dimethylsulfoxide and was cooled to 0° C. Potassium carbonate (5 equiv) followed by 30% hydrogen peroxide (2 equiv) was added and the reaction mixture was stirred at room temperature for 1 h. The reaction was diluted with ethyl acetate and the organic layer was washed with water, brine, dried over anhydrous sodium sulfate, filtered and concentrated to afford compound A145.

¹H NMR (400 MHz, DMSO-d₆) δ 8.60 (d, J=4.8 Hz, 1H), 8.23 (d, J=8.8 Hz, 1H), 8.11 (s, 1H), 8.06 (s, 1H), 7.95 (s, 1H), 7.68-7.61 (m, 3H), 4.41-3.71 (m, 5H), 3.41-3.30 (m, 3H), 3.25-3.02 (m, 6H), 2.32-2.26 (m, 1H), 2.22-2.12 (m, 1H), 1.60-1.55 (m, 2H), 1.18-1.10 (m, 3H), 0.89 (t, J=7.4 Hz, 3H).

LCMS (ESI-TOF) m/z 550.2 [M+H⁺] with a purity of >99%.

### Propyl 4-(5-allyl-9-chloro-5,6,7,8-tetrahydroacridine-3-carbonyl)piperazine-1-carboxylate (A146)

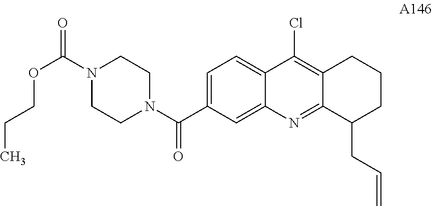 [SMILES: C=CCC1CCCc2c1nc1cc(C(=O)N3CCN(C(=O)OCCC)CC3)ccc1c2Cl]

Compound A146 was synthesized via General Procedure A, B and C1 using 2-allylcyclohexanone (General Procedure A) and n-propyl piperazine-1-carboxylate (General Procedure C1) as reagents.

¹H NMR (400 MHz, DMSO-d6) δ 8.19 (d, J=8.6 Hz, 1H), 7.97 (d, J=11.4 Hz, 1H), 7.67 (d, J=8.5 Hz, 1H), 5.88 (td, J=16.9, 7.9 Hz, 1H), 5.08 (dd, J=22.3, 13.6 Hz, 2H), 3.98 (t, J=6.6 Hz, 2H), 3.55 (d, J=99.3 Hz, 8H), 2.97 (dd, J=63.8, 32.0 Hz, 4H), 2.48-2.40 (m, 1H), 1.98 (s, 2H), 1.84-1.62 (m, 2H), 1.58 (dd, J=13.9, 6.9 Hz, 2H), 0.89 (t, J=7.3 Hz, 3H).

LCMS (ESI-TOF) m/z 456.2 [M+H⁺] with a purity of >96%.

### Propyl 4-(9-chloro-6-(5-ethynylpyridin-2-yl)-5,6,7,8-tetrahydroacridine-3-carbonyl)piperazine-1-carboxylate (A147)

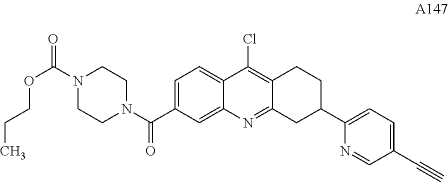 [SMILES: C#Cc1ccc(C2CCc3c(nc4cc(C(=O)N5CCN(C(=O)OCCC)CC5)ccc4c3Cl)C2)nc1]

Compound A147 was prepared according to General Procedure E, F, C2 and G using 3-(5-ethynylpyridin-2-yl)cyclohexanone (General Procedure E) and n-propyl piperazine-1-carboxylate (General Procedure C2) as starting materials.

¹H NMR (400 MHz, DMSO-d₆) δ 8.64 (d, J=1.6 Hz, 1H), 8.22 (d, J=8.4 Hz, 1H), 7.97 (s, 1H), 7.89 (dd, J=2.0, 8.4 Hz, 1H), 7.67 (d, J=8.8 Hz, 1H), 7.47 (d, J=7.6 Hz, 1H), 4.39 (s, 1H), 3.97 (t, J=6.8 Hz, 2H), 3.75-2.90 (m, 13H), 2.30 (br s, 1H), 2.10 (br s, 1H), 1.65-1.50 (m, 2H), 0.89 (t, J=7.2 Hz, 3H)

LCMS (ESI-TOF) m/z 517.3 [M+H⁺] with a purity of >98%.

### Propyl 4-(9-chloro-6-(2-cyanopyrimidin-4-yl)-5,6,7,8-tetrahydroacridine-3-carbonyl)piperazine-1-carboxylate (A148)

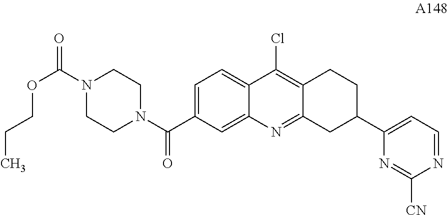 [SMILES: CCCOC(=O)N1CCN(C(=O)c2ccc3c(Cl)c4c(nc3c2)CC(c2ccnc(C#N)n2)CC4)CC1]

Compound A148 was prepared according to General Procedure A, B and C2 using 4-(3-oxocyclohexyl)pyrimidine-2-carbonitrile (General Procedure A) and n-propyl piperazine-1-carboxylate (General Procedure C2) as starting materials.

¹H NMR (400 MHz, DMSO-d₆) δ 8.98 (d, J=5.2 Hz, 1H), 8.22 (d, J=8.8 Hz, 1H), 7.98-7.94 (m, 2H), 7.68 (dd, J=1.2, 8.8 Hz, 1H), 3.99-3.95 (m, 2H), 3.80-3.40 (m, 10H), 3.30-2.90 (m, 3H), 2.40-1.90 (m, 2H), 1.58 (dd, J=6.8, 14.4 Hz, 2H), 0.897 (t, J=7.2 Hz, 3H).

LCMS (ESI-TOF) m/z 519.3 [M+H⁺] with a purity of >96%.

### Propyl (2R)-4-(9-chloro-6-(5-ethynylpyridin-2-yl)-5,6,7,8-tetrahydroacridine-3-carbonyl)-2-methylpiperazine-1-carboxylate (A149)

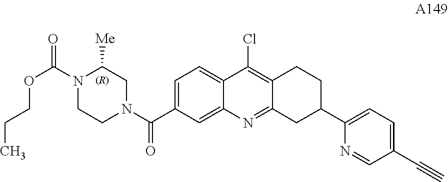

Compound A149 was prepared according to General Procedure E, F, C2 and G using 3-(5-ethynylpyridin-2-yl)cyclohexanone (General Procedure E) and (R)-n-propyl 2-methylpiperazine-1-carboxylate (General Procedure C2) as starting materials.

¹H NMR (400 MHz, DMSO-d₆) δ 8.64 (d, J=1.6 Hz, 1H), 8.22 (d, J=8.8 Hz, 1H), 7.95 (br s, 1H), 7.90 (dd, J=2.4, 8.0 Hz, 1H), 7.68 (br s, 1H), 7.47 (d, J=7.6 Hz, 1H), 4.39 (s, 1H), 4.40-2.90 (m, 14H), 2.27 (br s, 1H), 2.15-2.05 (m, 1H), 1.65-1.50 (m, 2H), 1.40-1.10 (m, 3H), 0.88 (t, J=7.2 Hz, 3H).

LCMS (ESI-TOF) m/z 531.6 [M+H⁺] with a purity of >98%.

### Propyl (3S)-4-(9-chloro-6-(5-ethynylpyridin-2-yl)-5,6,7,8-tetrahydroacridine-3-carbonyl)-3-methylpiperazine-1-carboxylate (A150)

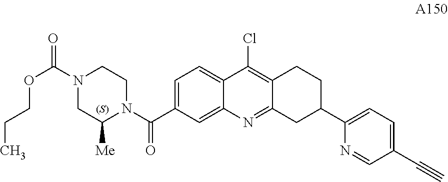

Compound A150 was prepared according to General Procedure E, F, C2 and G using 3-(5-ethynylpyridin-2-yl)cyclohexanone (General Procedure E) and (S)-n-propyl 3-methylpiperazine-1-carboxylate (General Procedure C2) as starting materials.

¹H NMR (400 MHz, DMSO-d₆) δ 8.64 (s, 1H), 8.22 (d, J=8.8 Hz, 1H), 7.95 (s, 1H), 7.89 (dd, J=2.0 , 8.4 Hz, 1H), 7.65 (d, J=8.8 Hz, 1H), 7.47 (d, J=7.6 Hz, 1H), 4.39 (s, 1H), 4.10-3.60 (m, 5H), 3.50-2.90 (m, 9H), 2.27 (br s, 1H), 2.09 (br s, 1H), 1.65-1.50 (m, 2H), 1.17 (br s, 3H), 0.89 (t, J=7.2 Hz, 3H).

LCMS (ESI-TOF) m/z 531.5 [M+H⁺] with a purity of >99%.

### Propyl 4-(9-chloro-6-(1-methyl-1H-pyrazol-4-yl)-5,6,7,8-tetrahydroacridine-3-carbonyl)piperazine-1-carboxylate (A151)

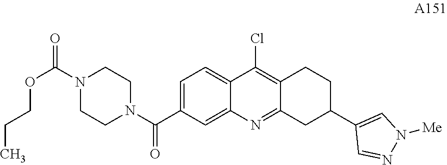 [SMILES: CCCOC(=O)N1CCN(C(=O)c2ccc3c(Cl)c4c(nc3c2)CC(c2cnn(C)c2)CC4)CC1]

Compound A148 was prepared according to General Procedure A, B and C2 using 3-(1-methyl-1H-pyrazol-4-yl)cyclohexanone (General Procedure A) and n-propyl piperazine-1-carboxylate (General Procedure C2) as starting materials.

¹H NMR (400 MHz, DMSO-d₆) δ 8.20 (d, J=8.4 Hz, 1H), 7.97 (d, J=1.6 Hz, 1H), 7.68 (dd, J=1.6, 8.4 Hz, 1H), 7.60 (s, 1H), 7.39 (s, 1H), 3.97 (t, J=6.4 Hz, 2H), 3.78 (s, 3H), 3.70-3.36 (m, 9H), 3.29-3.01 (m, 4H), 2.30-2.24 (m, 1H), 1.90-1.70 (m, 1H), 1.61-1.55 (m, 2H), 0.88 (t, J=6.8 Hz, 3H).

LCMS (ESI-TOF) m/z 496.3 [M+H⁺] with a purity of >95%.

### Propyl 4-(4-chloro-2-(pyridin-3-yl)quinoline-7-carbonyl)piperazine-1-carboxylate (B001)

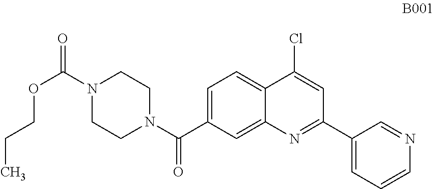 [SMILES: CCCOC(=O)N1CCN(C(=O)c2ccc3c(Cl)cc(-c4cccnc4)nc3c2)CC1]

Compound B001 was prepared according to General Procedure H, K, J and C2 using ethyl 3-oxo-3-(pyridin-3-yl)propanoate (General Procedure H) and n-propyl piperazine-1-carboxylate (General Procedure C2) as starting materials.

¹H NMR (400 MHz, DMSO-d6) δ 9.49 (d, J=2.0 Hz, 1H), 8.74-8.73 (m, 1H) 8.69-8.66 (m, 1H), 8.59 (s, 1H), 8.32-8.30 (d, J=8.4 Hz, 1H), 8.18 (d, J=1.2 Hz, 1H), 7.78 (dd, J=1.6, 8.8 Hz, 1H), 7.63-7.59 (m, 1H), 3.97 (t, J=6.6 Hz, 2H), 3.70 (br s, 2H), 3.51-3.36 (m, 6H), 1.61-1.56 (m, 2H), 0.89 (t, J=7.4 Hz, 3H).

LCMS (ESI-TOF) m/z 439.4 [M+H⁺] with a purity of >99%.

### Propyl 4-(4-chloro-2-phenylquinoline-7-carbonyl)piperazine-1-carboxylate (B002)

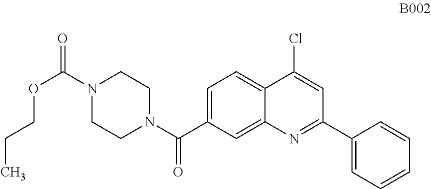 [SMILES: CCCOC(=O)N1CCN(C(=O)c2ccc3c(Cl)cc(-c4ccccc4)nc3c2)CC1]

Compound B002 was prepared according to General Procedure H, K, J and C2 using ethyl 3-oxo-3-phenylpropanoate (General Procedure H) and n-propylpiperazine-1-carboxylate (General Procedure C2) as starting materials.

¹H NMR (400 MHz, DMSO-d₆) δ 8.49 (s, 1H), 8.34-8.27 (m, 3H), 8.15 (d, J=0.8 Hz, 1H), 7.75 (dd, J=1.6, 8.8 Hz, 1H), 7.57 (dd, J=5.6, 13.2 Hz, 3H), 3.97 (t, J=6.6 Hz, 2H), 3.70-3.37 (m, 8H), 1.59-1.57 (m, 2H), 0.89 (t, J=7.4 Hz, 3H).

LCMS (ESI-TOF) m/z 438.2 [M+H⁺] with a purity of >98%.

### Propyl 4-(4-chloro-3-methyl-2-phenylquinoline-7-carbonyl)piperazine-1-carboxylate (B003)

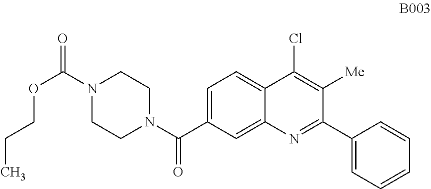 [SMILES: CCCOC(=O)N1CCN(C(=O)c2ccc3c(Cl)c(C)c(-c4ccccc4)nc3c2)CC1]

Compound B003 was prepared according to General Procedure I, K and C1 using propiophenone (General Procedure I) and n-propyl piperazine-1-carboxylate (General Procedure C1) as starting materials.

¹H NMR (400 MHz, DMSO-d₆) δ 8.32 (d, J=8.8 Hz, 1H), 8.11 (s, 1H), 7.68 (dd, J=8.8, 1.6 Hz, 1H), 7.57-7.47 (m, 5H), 4.06 (t, J=6.4 Hz, 2H), 3.81-3.45 (m, 8H), 2.55 (s, 3H), 1.69-1.63 (m, 2H), 0.95 (t, J=7.6 Hz, 3H).

LCMS (ESI-TOF) m/z 452.2 [M+H⁺] with a purity of >99%.

### Propyl 4-(4-chloro-3-ethyl-2-phenylquinoline-7-carbonyl)piperazine-1-carboxylate (B004)

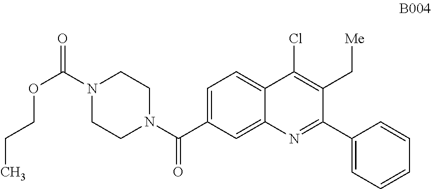 [SMILES: CCCOC(=O)N1CCN(C(=O)c2ccc3c(Cl)c(CC)c(-c4ccccc4)nc3c2)CC1]

Compound B004 was prepared similar to B003 using the 1-phenylbutan-1-one and n-propyl piperazine-1-carboxylate (General Procedure C2) as starting materials.

¹H NMR (400 MHz, CDCl₃) δ 8.34 (d, J=8.4 Hz, 1H), 8.10 (d, J=1.2 Hz, 1H), 7.68 (dd, J=1.6, 8.4 Hz, 1H), 7.50-7.40 (m, 5H), 4.06 (t, J=6.4 Hz, 2H), 3.91-3.30 (m, 8H), 2.98-2.93 (m, 2H), 1.69-1.52 (m, 2H), 1.17-1.13 (m, 3H), 0.95 (t, J=7.2 Hz, 3H).

LCMS (ESI-TOF) m/z 466.3 [M+H⁺] with a purity of >99%.

### Propyl 4-(4-chloro-2-(5-methylpyridin-3-yl)quinoline-7-carbonyl)piperazine-1-carboxylate (B005)

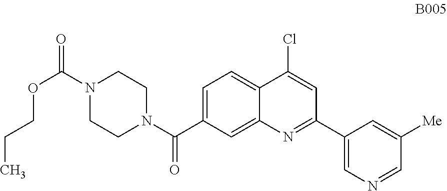 [SMILES: CCCOC(=O)N1CCN(C(=O)c2ccc3c(Cl)cc(-c4cncc(C)c4)nc3c2)CC1]

Compound B005 was prepared according to General Procedure H, K, J and C2 using ethyl 4-(5-methylpyridin-3-yl)-3-oxobutanoate (General Procedure H) and n-propyl piperazine-1-carboxylate (General Procedure C2) as starting materials.

¹H NMR (400 MHz, DMSO-d₆) δ 9.29 (d, J=2.0 Hz, 1H), 8.57 (s, 2H), 8.51 (s, 1H), 8.30 (d, J=8.8 Hz, 1H), 8.18 (d, J=0.8 Hz, 1H), 7.78 (dd, J=1.2, 8.8 Hz, 1H), 3.98 (t, J=6.4 Hz, 2H), 3.72-3.32 (m, 8H), 2.44 (s, 3H), 1.61-1.56 (m, 2H), 0.89 (t, J=7.4 Hz, 3H).

LCMS (ESI-TOF) m/z 453.2 [M+H⁺] with a purity of >97%.

### Propyl (S)-4-(4-chloro-3-methyl-2-phenylquinoline-7-carbonyl)-2-methylpiperazine-1-carboxylate (B006)

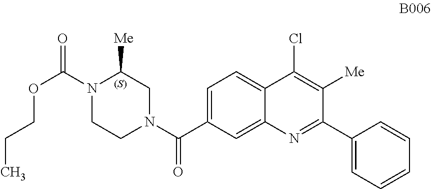

Intermediate from General Procedure K in the synthesis of B003 was subjected to General Procedure C1 with (S)-n-propyl 2-methylpiperazine-1-carboxylate as reagent to afford B006.

¹H NMR (400 MHz, 80° C., DMSO-d₆) δ 8.28 (d, J=8.6 Hz, 1H), 8.02 (s, 1H), 7.72 (d, J=8.6 Hz, 1H), 7.60 (d, J=6.8 Hz, 2H), 7.56-7.47 (m, 3H), 4.23 (s, 1H), 3.98 (pd, J=10.5, 6.6 Hz, 3H), 3.81 (d, J=11.7 Hz, 1H), 3.32-3.07 (m, 4H), 2.50 (d, J=4.5 Hz, 3H), 1.65-1.50 (m, 2H), 1.12 (d, J=6.5 Hz, 3H), 0.89 (t, J=7.4 Hz, 3H).

LCMS (ESI-TOF) m/z 466.2 [M+H⁺] with a purity of >97%.

### Propyl (R)-4-(4-chloro-3-methyl-2-phenylquinoline-7-carbonyl)-2-methylpiperazine-1-carboxylate (B007)

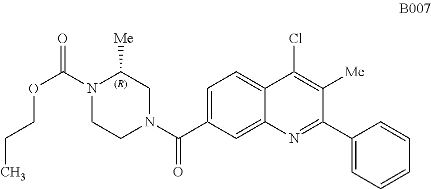

Intermediate from General Procedure K in the synthesis of B003 was subjected to General Procedure C1 with (R)-n-propyl 2-methylpiperazine-1-carboxylate as reagent to afford B007.

¹H NMR (400 MHz, 80° C., DMSO-d₆) δ 8.28 (d, J=8.6 Hz, 1H), 8.02 (s, 1H), 7.72 (d, J=8.6 Hz, 1H), 7.60 (d, J=7.6 Hz, 2H), 7.56-7.46 (m, 3H), 4.23 (s, 1H), 3.99 (pd, J=10.5, 6.4 Hz, 3H), 3.81 (d, J=11.1 Hz, 1H), 3.36-3.07 (m, 4H), 2.50 (d, J=5.8 Hz, 3H), 1.68-1.51 (m, 2H), 1.12 (d, J=6.6 Hz, 3H), 0.89 (t, J=7.4 Hz, 3H).

LCMS (ESI-TOF) m/z 466.2 [M+H⁺] with a purity of >96%.

### Propyl 4-(4-chloro-2-(pyridin-2-yl)quinoline-7-carbonyl)piperazine-1-carboxylate (B008)

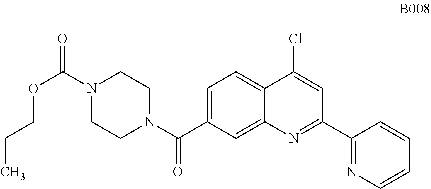 [SMILES: CCCOC(=O)N1CCN(C(=O)c2ccc3c(Cl)cc(-c4ccccn4)nc3c2)CC1]

Compound B008 was prepared according to General Procedure H, K, J and C2 using ethyl 3-oxo-3-(pyridin-2-yl)propanoate (General Procedure H) and n-propyl piperazine-1-carboxylate (General Procedure C2) as starting materials.

¹H NMR (400 MHz, DMSO-d₆) δ 8.79 (d, J=4.4 Hz, 1H), 8.72 (s, 1H), 8.60 (d, J=8.4 Hz, 1H), 8.32 (d, J=8.4 Hz, 1H), 8.20 (m, 1H), 8.09-8.04 (m, 1H), 7.80 (dd, J=1.6, 8.8 Hz, 1H), 7.60-7.57 (m, 1H), 3.98 (t, J=6.6 Hz, 2H), 3.76-3.35 (m, 8H), 1.61-1.56 (m, 2H), 0.89 (t, J=7.2 Hz, 3H).

LCMS (ESI-TOF) m/z 439.5 [M+H⁺] with a purity of >98%.

### Propyl 4-(2-benzyl-4-chloroquinoline-7-carbonyl)piperazine-1-carboxylate (B009)

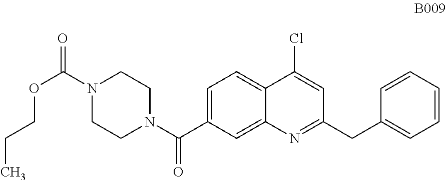 [SMILES: CCCOC(=O)N1CCN(C(=O)c2ccc3c(Cl)cc(Cc4ccccc4)nc3c2)CC1]

Compound B009 was prepared according to General Procedure H, K, J and C2 using ethyl 3-oxo-5-phenylpentanoate (General Procedure H) and n-propyl piperazine-1-carboxylate (General Procedure C2) as starting materials.

¹H NMR (400 MHz, DMSO-d₆) δ 8.21 (d, J=8.0 Hz, 1H), 8.04 (m, 1H), 7.76 (s, 1H), 7.71 (dd, J=7.2, 1.6 Hz, 1H), 7.37-7.29 (m, 4H), 7.24-7.20 (m, 1H), 4.29 (s, 2H), 3.97 (t, J=6.6 Hz, 2H), 3.82-3.32 (m, 8H), 1.61-1.55 (m, 2H), 0.88 (t, J=7.0 Hz, 3H).

LCMS (ESI-TOF) m/z 452.2 [M+H⁺] with a purity of >95%.

### Propyl 4-(4-chloro-2-(mtolyl)quinoline-7-carbonyl)piperazine-1-carboxylate (B010)

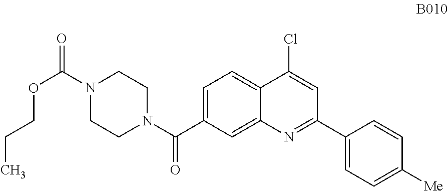 [SMILES: CCCOC(=O)N1CCN(C(=O)c2ccc3c(Cl)cc(-c4ccc(C)cc4)nc3c2)CC1]

Compound B010 was prepared according to General Procedure H, K, J and C2 using ethyl 3-oxo-3-p-tolylpropanoate (General Procedure H) and n-propyl piperazine-1-carboxylate (General Procedure C2) as starting materials.

¹H NMR (400 MHz, DMSO-d₆) δ 8.45 (s, 1H), 8.27-8.23 (m, 3H), 8.12 (d, J=0.8 Hz, 1H), 7.73 (dd, J=1.6, 8.8 Hz, 1H), 7.38 (d, J=8.0 Hz, 2H), 3.98 (t, J=6.4 Hz, 2H), 3.69-3.35(m, 8H), 2.40 (s, 3H), 1.61-1.56 (m, 2H), 0.89 (t, J=7.4 Hz, 3H).

LCMS (ESI-TOF) m/z 452.2 [M+H⁺] with a purity of >99%.

### Propyl 4-(4-chloro-2-(m-tolyl)quinoline-7-carbonyl)piperazine-1-carboxylate (B011)

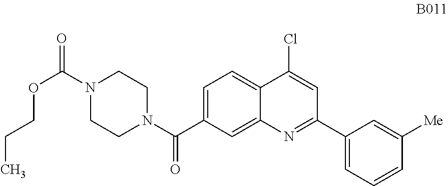 [SMILES: CCCOC(=O)N1CCN(C(=O)c2ccc3c(Cl)cc(-c4cccc(C)c4)nc3c2)CC1]

Compound B011 was prepared according to General Procedure H, K, J and C2 using ethyl 3-oxo-3-m-tolylpropanoate (General Procedure H) and n-propyl piperazine-1-carboxylate (General Procedure C2) as starting materials.

¹H NMR (400 MHz, DMSO-d₆) δ 8.46 (s, 1H), 8.27 (d, J=8.4 Hz, 1H), 8.16-8.10 (m, 3H), 7.74 (dd, J=1.2, 8.4 Hz, 1H), 7.46 (t, J=7.6 Hz, 1H), 7.36 (d, J=7.2 Hz, 1H), 3.98 (t, J=6.6 Hz, 2H), 3.70-3.36 (m, 8H), 2.44 (s, 3H), 1.59 (dd, J=6.8, 13.6 Hz, 2H), 0.89 (t, J=7.4 Hz, 3H).

LCMS (ESI-TOF) m/z 452.2 [M+H⁺] with a purity of >99%.

### Propyl 4-(4-chloro-2-(o-tolyl)quinoline-7-carbonyl)piperazine-1-carboxylate (B012)

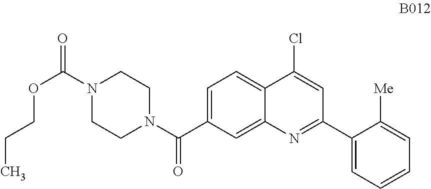 [SMILES: CCCOC(=O)N1CCN(C(=O)c2ccc3c(Cl)cc(-c4ccccc4C)nc3c2)CC1]

Compound B012 was prepared according to General Procedure H, K, J and C2 using ethyl 3-oxo-4-o-tolylbutanoate (General Procedure H) and n-propyl piperazine-1-carboxylate (General Procedure C2) as starting materials.

¹H NMR (400 MHz, DMSO-d₆) δ 8.31 (d, J=8.4 Hz, 1H), 8.12 (s, 1H), 8.03 (s, 1H), 7.79 (d, J=8.4 Hz, 1H), 7.55 (d, J=7.2 Hz, 1H), 7.45-7.30 (m, 3H), 3.97 (t, J=6.6 Hz, 2H), 3.75-3.35 (m, 8H), 2.41 (s, 3H), 1.65-1.50 (m, 2H), 0.89 (t, J=6.8 Hz, 3H).

LCMS (ESI-TOF) m/z 452.2 [M+H⁺] with a purity of >98%.

### Propyl 4-(4-chloro-2-(pyridin-4-yl)quinoline-7-carbonyl)piperazine-1-carboxylate (B013)

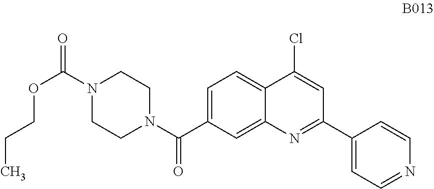 [SMILES: CCCOC(=O)N1CCN(C(=O)c2ccc3c(Cl)cc(-c4ccncc4)nc3c2)CC1]

Compound B013 was prepared according to General Procedure H, K, J and C2 using ethyl 3-oxo-3-(pyridin-4-yl)propanoate (General Procedure H) and n-propyl piperazine-1-carboxylate (General Procedure C2) as starting materials.

¹H NMR (400 MHz, DMSO-d₆) δ 8.80 (d, J=6.0 Hz, 2H), 8.61 (s, 1H), 8.33 (d, J=8.8 Hz, 1H), 8.28 (dd, J=1.6 , 4.4 Hz, 2H), 8.20 (s, 1H), 7.82 (dd, J=1.6, 8.8 Hz, 1H), 3.98 (t, J=6.6 Hz, 2H), 3.81-3.34 (m, 8H), 1.61-1.56 (m, 2H), 0.89 (t, J=7.4 Hz, 3H).

LCMS (ESI-TOF) m/z 439.2 [M+H⁺] with a purity of >98%.

### Propyl (S)-4-(4-chloro-2-phenylquinoline-7-carbonyl)-3-methylpiperazine-1-carboxylate (B014)

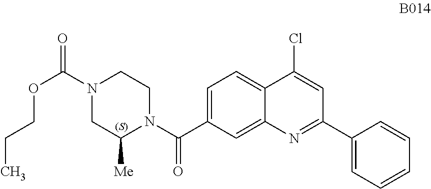

Intermediate from General Procedure K in the synthesis of B002 was subjected to General Procedure C1 with (S)-n-propyl 3-methylpiperazine-1-carboxylate as reagent to afford B014.

¹H NMR (400 MHz, 80° C., DMSO-d₆) δ 8.37 (s, 1H), 8.33-8.23 (m, 3H), 8.10 (s, 1H), 7.70 (d, J=8.3 Hz, 1H), 7.62-7.49 (m, 3H), 4.36 (br s, 1H), 4.12-3.73 (m, 6H), 3.28-3.16 (m, 2H), 1.60 (dd, J=14.2, 7.0 Hz, 2H), 1.21 (d, J=6.8 Hz, 3H), 0.90 (t, J=7.4 Hz, 3H).

LCMS (ESI-TOF) m/z 452.1 [M+H⁺] with a purity of >98%.

### Propyl (R)-4-(4-chloro-2-phenylquinoline-7-carbonyl)-3-methylpiperazine-1-carboxylate (B015)

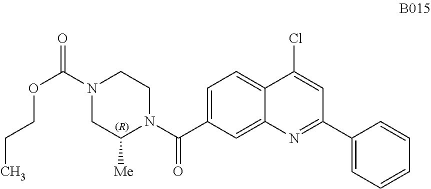

Intermediate from General Procedure K in the synthesis of B002 was subjected to General Procedure C1 with (R)-n-propyl 3-methylpiperazine-1-carboxylate as reagent to afford B015.

¹H NMR (400 MHz, 80° C., DMSO-d₆) δ 8.37 (s, 1H), 8.32-8.24 (m, 3H), 8.10 (s, 1H), 7.70 (d, J=8.6 Hz, 1H), 7.55 (p, J=6.1 Hz, 3H), 4.37 (br s, 1H), 4.08-3.72 (m, 6H), 3.34-3.13 (m, 2H), 1.65-1.53 (m, 2H), 1.21 (d, J=6.7 Hz, 3H), 0.90 (t, J=7.4 Hz, 3H).

LCMS (ESI-TOF) m/z 452.1 [M+H⁺] with a purity of >98%.

### Propyl (S)-4-(4-chloro-3-methyl-2-phenylquinoline-7-carbonyl)-3-methylpiperazine-1-carboxylate (B016)

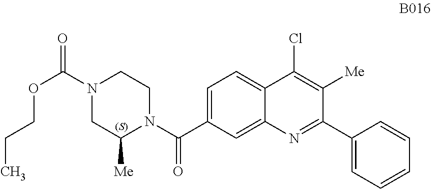

Intermediate from General Procedure K in the synthesis of B003 was subjected to General Procedure C1 with (S)-n-propyl 3-methylpiperazine-1-carboxylate as reagent to afford B016.

¹H NMR (400 MHz, 80° C., DMSO-d₆) δ 8.37 (s, 1H), 8.33-8.23 (m, 2H), 8.10 (s, 1H), 7.70 (d, J=8.3 Hz, 1H), 7.62-7.49 (m, 3H), 4.36 (br s, 1H), 4.12-3.73 (m, 6H), 3.28-3.16 (m, 2H), 2.51 (s, 3H), 1.60 (dd, J=14.2, 7.0 Hz, 2H), 1.21 (d, J=6.8 Hz, 3H), 0.90 (t, J=7.4 Hz, 3H).

LCMS (ESI-TOF) m/z 466.2 [M+H⁺] with a purity of >95%.

### Propyl (R)-4-(4-chloro-3-methyl-2-phenylquinoline-7-carbonyl)-3-methylpiperazine-1-carboxylate (B017)

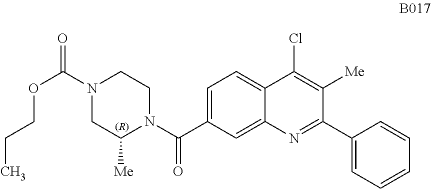

Intermediate from General Procedure K in the synthesis of B003 was subjected to General Procedure C1 with (R)-n-propyl 3-methylpiperazine-1-carboxylate as reagent to afford B017.

¹H NMR (400 MHz, 80° C., DMSO-d₆) δ 8.28 (d, J=8.6 Hz, 1H), 8.01 (s, 1H), 7.70 (d, J=8.7 Hz, 1H), 7.60 (d, J=7.0 Hz, 2H), 7.56-7.46 (m, 3H), 4.36 (br s, 1H), 4.04-3.72 (m, 6H), 3.27-3.12 (m, 2H), 2.51 (s, 3H), 1.59 (dq, J=13.8, 6.9 Hz, 2H), 1.19 (d, J=6.7 Hz, 3H), 0.89 (t, J=7.4 Hz, 3H).

LCMS (ESI-TOF) m/z 466.2 [M+H⁺] with a purity of >97%.

### Propyl (S)-4-(4-chloro-2-phenylquinoline-7-carbonyl)-2-methylpiperazine-1-carboxylate (B018)

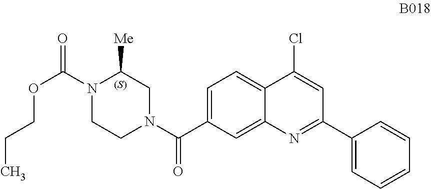

Intermediate from General Procedure K in the synthesis of B002 was subjected to General Procedure C1 with (S)-n-propyl 2-methylpiperazine-1-carboxylate as reagent to afford B018.

¹H NMR (400 MHz, 80° C., DMSO-d₆) δ 8.38 (s, 1H), 8.29 (dd, J=7.5, 3.3 Hz, 3H), 8.11 (s, 1H), 7.72 (d, J=8.5 Hz, 1H), 7.60-7.50 (m, 3H), 4.25 (s, 1H), 3.99 (pd, J=10.6, 6.6 Hz, 3H), 3.82 (d, J=12.5 Hz, 1H), 3.38-3.08 (m, 4H), 1.66-1.51 (m, 2H), 1.14 (d, J=6.3 Hz, 3H), 0.89 (t, J=7.4 Hz, 3H).

LCMS (ESI-TOF) m/z 452.1 [M+H⁺] with a purity of >97%.

### Propyl (R)-4-(4-chloro-2-phenylquinoline-7-carbonyl)-2-methylpiperazine-1-carboxylate (B019)

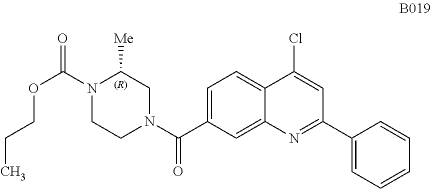

Intermediate from General Procedure K in the synthesis of B002 was subjected to General Procedure C1 with (R)-n-propyl 2-methylpiperazine-1-carboxylate as reagent to afford B019.

¹H NMR (400 MHz, 80° C., DMSO-d₆) δ 8.38 (s, 1H), 8.32-8.23 (m, 3H), 8.11 (s, 1H), 7.72 (d, J=8.6 Hz, 1H), 7.56 (q, J=6.6 Hz, 3H), 4.25 (s, 1H), 3.99 (pd, J=10.6, 6.5 Hz, 3H), 3.82 (d, J=12.6 Hz, 1H), 3.36-3.06 (m, 4H), 1.65-1.51 (m, 2H), 1.14 (d, J=6.6 Hz, 3H), 0.89 (t, J=7.4 Hz, 3H).

LCMS (ESI-TOF) m/z 452.1 [M+H⁺] with a purity of >95%.

### Propyl 4-(4-chloro-3-methyl-2-(4-(trifluoromethyl)phenyl)quinoline-7-carbonyl)piperazine-1-carboxylate (B020)

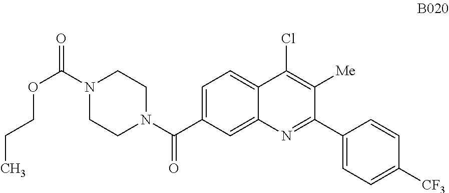 [SMILES: CCCOC(=O)N1CCN(C(=O)c2ccc3c(Cl)c(C)c(-c4ccc(C(F)(F)F)cc4)nc3c2)CC1]

Compound B020 was synthesized according to General Procedure I, K and C1 using 4-trifluoromethylpropiophenone (General Procedure I) and n-propyl piperazine-1-carboxylate (General Procedure C1) as starting materials.

¹H NMR (400 MHz, DMSO-d₆) δ 8.31 (d, J=8.6 Hz, 1H), 8.08 (d, J=1.1 Hz, 1H), 7.89 (dd, J=19.3, 8.4 Hz, 4H), 7.79 (dd, J=8.6, 1.5 Hz, 1H), 3.97 (t, J=6.6 Hz, 2H), 3.73-3.36 (m, 8H), 2.51 (s, 3H), 1.64-1.50 (m, 2H), 0.89 (t, J=7.2 Hz, 3H).

LCMS (ESI-TOF) m/z 520.1 [M+H⁺] with a purity of >98%.

### Propyl 4-(4-chloro-2-(4-chlorophenyl)-3-methylquinoline-7-carbonyl)piperazine-1-carboxylate (B021)

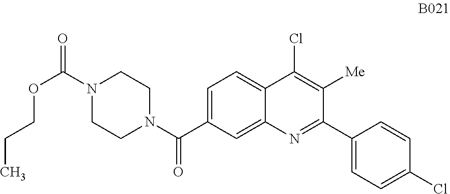 [SMILES: CCCOC(=O)N1CCN(C(=O)c2ccc3c(Cl)c(C)c(-c4ccc(Cl)cc4)nc3c2)CC1]

Compound B021 was synthesized according to General Procedure I, K and C1 using 4-chloropropiophenone (General Procedure I) and n-propyl piperazine-1-carboxylate (General Procedure C1) as starting materials.

¹H NMR (400 MHz, DMSO-d₆) δ 8.29 (d, J=8.6 Hz, 1H), 8.07 (d, J=1.3 Hz, 1H), 7.77 (dd, J=8.6, 1.5 Hz, 1H), 7.64 (dd, J=27.1, 8.5 Hz, 4H), 3.97 (t, J=6.6 Hz, 2H), 3.79-3.36 (m, 8H), 1.66-1.49 (m, 2H), 0.89 (t, J=7.3 Hz, 3H).

LCMS (ESI-TOF) m/z 486.1 [M+H⁺] with a purity of >96%.

### Propyl 4-(4-chloro-2-(4-fluorophenyl)-3-methylquinoline-7-carbonyl)piperazine-1-carboxylate (B022)

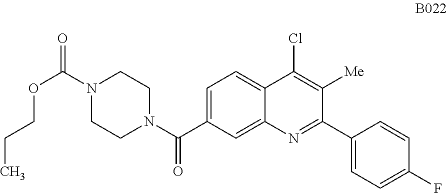 [SMILES: CCCOC(=O)N1CCN(C(=O)c2ccc3c(Cl)c(C)c(-c4ccc(F)cc4)nc3c2)CC1]

Compound B022 was synthesized according to General Procedure I, K and C1 using 4-fluoropropiophenone (General Procedure I) and n-propyl piperazine-1-carboxylate (General Procedure C1) as starting materials.

¹H NMR (400 MHz, 80° C., DMSO-d₆) δ 8.27 (d, J=8.6 Hz, 1H), 8.04 (s, 1H), 7.73 (d, J=8.5 Hz, 1H), 7.70-7.62 (m, 2H), 7.33 (t, J=8.9 Hz, 2H), 3.99 (t, J=6.5 Hz, 2H), 3.62-3.37 (m, 8H), 2.51 (s, 3H), 1.70-1.52 (m, 2H), 0.89 (t, J=7.4 Hz, 3H).

LCMS (ESI-TOF) m/z 470.1 [M+H⁺] with a purity of >98%.

### Propyl (R)-4-(4-chloro-3-methyl-2-(p-tolyl)quinoline-7-carbonyl)-3-methylpiperazine-1-carboxylate (B023)

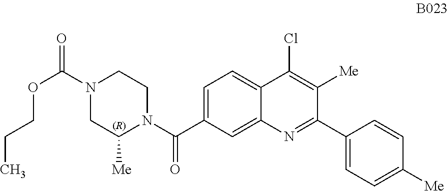

Compound B023 was synthesized according to General Procedure I, K and C1 using 4-methylpropiophenone (General Procedure I) and (R)-n-propyl 3-methylpiperazine-1-carboxylate (General Procedure C1) as starting materials.

¹H NMR (400 MHz, DMSO-d₆) δ 8.26 (d, J=8.6 Hz, 1H), 7.99 (s, 1H), 7.69 (d, J=8.6 Hz, 1H), 7.50 (d, J=7.7 Hz, 2H), 7.33 (d, J=7.8 Hz, 2H), 4.35 (s, 1H), 4.08-3.68 (m, 5H), 3.29-3.12 (m, 2H), 3.02-2.89 (m, 1H), 2.51 (s, 3H), 2.41 (s, 3H), 1.67-1.45 (m, 2H), 1.19 (d, J=6.7 Hz, 3H), 0.89 (t, J=7.4 Hz, 3H).

LCMS (ESI-TOF) m/z 480.2 [M+H⁺] with a purity of >99%.

### Propyl (S)-4-(4-chloro-3-methyl-2-(p-tolyl)quinoline-7-carbonyl)-3-methylpiperazine-1-carboxylate (B024)

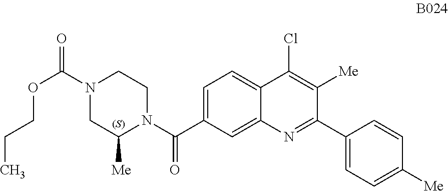

Compound B024 was synthesized according to General Procedure I, K and C1 using 4-methylpropiophenone (General Procedure I) and (S)-n-propyl 3-methylpiperazine-1-carboxylate (General Procedure C1) as starting materials.

¹H NMR (400 MHz, DMSO-d₆) δ 8.26 (d, J=8.6 Hz, 1H), 7.99 (s, 1H), 7.69 (d, J=8.7 Hz, 1H), 7.50 (d, J=7.9 Hz, 2H), 7.33 (d, J=7.8 Hz, 2H), 4.35 (s, 1H), 4.04-3.72 (m, 5H), 3.30-2.90 (m, 3H), 2.51 (s, 3H), 2.41 (s, 3H), 1.66-1.51 (m, 2H), 1.19 (d, J=6.7 Hz, 3H), 0.89 (t, J=7.4 Hz, 3H).

LCMS (ESI-TOF) m/z 480.2 [M+H⁺] with a purity of >97%.

### Propyl 4-(4-chloro-3-methyl-2-(p-tolyl)quinoline-7-carbonyl)piperazine-1-carboxylate (B025)

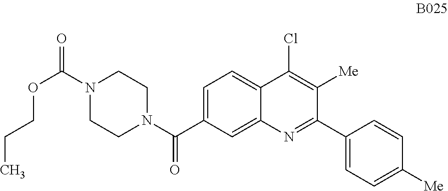 [SMILES: CCCOC(=O)N1CCN(C(=O)c2ccc3c(Cl)c(C)c(-c4ccc(C)cc4)nc3c2)CC1]

Compound B025 was synthesized according to General Procedure I, K and C1 using 4-methylpropiophenone (General Procedure I) and n-propyl piperazine-1-carboxylate (General Procedure C1) as starting materials.

¹H NMR (400 MHz, 80° C., DMSO-d₆) δ 8.26 (d, J=8.6 Hz, 1H), 8.03 (s, 1H), 7.72 (d, J=8.6 Hz, 1H), 7.50 (d, J=7.8 Hz, 2H), 7.33 (d, J=7.8 Hz, 2H), 3.99 (t, J=6.5 Hz, 2H), 3.64-3.34 (m, 8H), 2.51 (d, J=5.1 Hz, 3H), 2.41 (s, 3H), 1.64-1.50 (m, 2H), 0.89 (t, J=7.4 Hz, 3H).

LCMS (ESI-TOF) m/z 466.1 [M+H⁺] with a purity of >98%.

### Propyl (S)-4-(4-chloro-2-(4-methoxyphenyl)-3-methylquinoline-7-carbonyl)-3-methylpiperazine-1-carboxylate (B026)

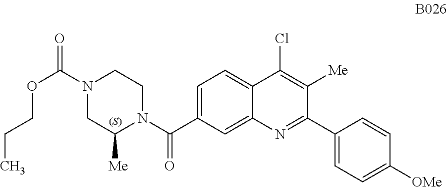

Compound B026 was synthesized according to General Procedure I, K and C1 using 4-methoxypropiophenone (General Procedure I) and (S)-n-propyl 3-methylpiperazine-1-carboxylate (General Procedure C1) as starting materials.

¹H NMR (400 MHz, DMSO-d₆) δ 8.24 (t, J=19.6 Hz, 1H), 8.01 (s, 1H), 7.71 (d, J=8.5 Hz, 1H), 7.60 (d, J=8.6 Hz, 2H), 7.09 (d, J=8.7 Hz, 2H), 4.09-3.55 (m, 9H), 3.26-2.79 (m, 3H), 2.55 (s, 3H), 1.65-1.48 (m, 2H), 1.17 (s, 3H), 0.89 (t, J=7.3 Hz, 3H).

LCMS (ESI-TOF) m/z 496.1 [M+H⁺] with a purity of >95%.

### Propyl 4-(4-chloro-3-methyl-2-(o-tolyl)quinoline-7-carbonyl)piperazine-1-carboxylate (B027)

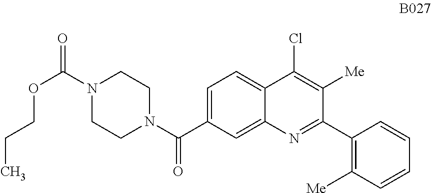 [SMILES: CCCOC(=O)N1CCN(C(=O)c2ccc3c(Cl)c(C)c(-c4ccccc4C)nc3c2)CC1]

Compound B027 was synthesized according to General Procedure I, K and C1 using 2-methylpropiophenone (General Procedure I) and n-propyl piperazine-1-carboxylate (General Procedure C1) as starting materials.

¹H NMR (400 MHz, DMSO-d₆) δ 8.30 (d, J=8.6 Hz, 1H), 8.05 (d, J=1.2 Hz, 1H), 7.78 (dd, J=8.6, 1.5 Hz, 1H), 7.46-7.30 (m, 3H), 7.27 (d, J=7.4 Hz, 1H), 3.97 (t, J=6.6 Hz, 2H), 3.76-3.35 (m, 8H), 2.30 (s, 3H), 2.04 (s, 3H), 1.65-1.49 (m, 2H), 0.88 (t, J=7.3 Hz, 3H).

LCMS (ESI-TOF) m/z 466.1 [M+H⁺] with a purity of >96%.

### Propyl 4-(4-chloro-3-methyl-2-(m-tolyl)quinoline-7-carbonyl)piperazine-1-carboxylate (B028)

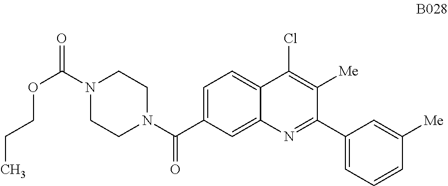 [SMILES: CCCOC(=O)N1CCN(C(=O)c2ccc3c(Cl)c(C)c(-c4cccc(C)c4)nc3c2)CC1]

Compound B028 was synthesized according to General Procedure I, K and C1 using 3-methylpropiophenone (General Procedure I) and n-propyl piperazine-1-carboxylate (General Procedure C1) as starting materials.

¹H NMR (400 MHz, DMSO-d₆) δ 8.28 (d, J=8.6 Hz, 1H), 8.06 (d, J=1.3 Hz, 1H), 7.75 (dd, J=8.6, 1.5 Hz, 1H), 7.47-7.36 (m, 3H), 7.33 (d, J=6.5 Hz, 1H), 3.97 (t, J=6.6 Hz, 2H), 3.75-3.34 (m, 8H), 2.49 (s, 3H), 2.41 (s, 3H), 1.65-1.51 (m, 2H), 0.89 (t, J=7.3 Hz, 3H).

LCMS (ESI-TOF) m/z 466.1 [M+H⁺] with a purity of >95%.

### Propyl 4-(4-chloro-2-(3-methoxyphenyl)-3-methylquinoline-7-carbonyl)piperazine-1-carboxylate (B029)

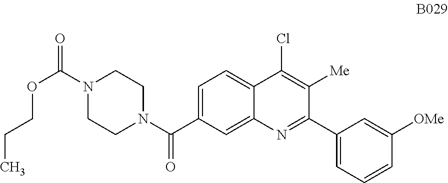 [SMILES: CCCOC(=O)N1CCN(C(=O)c2ccc3c(Cl)c(C)c(-c4cccc(OC)c4)nc3c2)CC1]

Compound B029 was synthesized according to General Procedure I, K and C1 using 3-methoxypropiophenone (General Procedure I) and n-propyl piperazine-1-carboxylate (General Procedure C1) as starting materials.

¹H NMR (400 MHz, 80° C., DMSO-d₆) δ 8.27 (d, J=8.6 Hz, 1H), 8.04 (s, 1H), 7.73 (d, J=8.6 Hz, 1H), 7.43 (t, J=7.8 Hz, 1H), 7.14 (d, J=7.0 Hz, 2H), 7.07 (d, J=9.9 Hz, 1H), 3.99 (t, J=6.6 Hz, 2H), 3.83 (s, 3H), 3.59-3.40 (m, 8H), 2.50 (s, 3H), 1.59 (dq, J=14.2, 7.0 Hz, 2H), 0.89 (t, J=7.4 Hz, 3H).

LCMS (ESI-TOF) m/z 482.1 [M+H⁺] with a purity of >96%.

### Propyl 4-(4-chloro-2-(2,3-difluorophenyl)-3-methylquinoline-7-carbonyl)piperazine-1-carboxylate (B030)

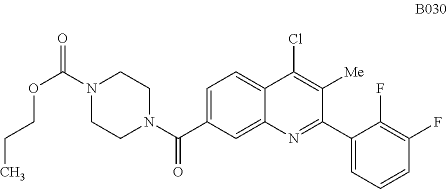 [SMILES: CCCOC(=O)N1CCN(C(=O)c2ccc3c(Cl)c(C)c(-c4cccc(F)c4F)nc3c2)CC1]

Compound B030 was synthesized according to General Procedure I, K and C1 using 2,3-difluoropropiophenone (General Procedure I) and n-propyl piperazine-1-carboxylate (General Procedure C1) as starting materials.

¹H NMR (400 MHz, DMSO-d₆) δ 8.33 (d, J=8.6 Hz, 1H), 8.10 (s, 1H), 7.82 (d, J=8.7 Hz, 1H), 7.70-7.55 (m, 1H), 7.42 (dd, J=9.6, 5.9 Hz, 2H), 3.97 (t, J=6.6 Hz, 2H), 3.77-3.50 (m, 8H), 2.42 (s, 3H), 1.64-1.52 (m, 2H), 0.88 (dd, J=13.4, 6.3 Hz, 3H).

LCMS (ESI-TOF) m/z 488.1 [M+H⁺] with a purity of >97%.

### Propyl 4-(4-chloro-2-(4-methoxyphenyl)-3-methylquinoline-7-carbonyl)piperazine-1-carboxylate (B031)

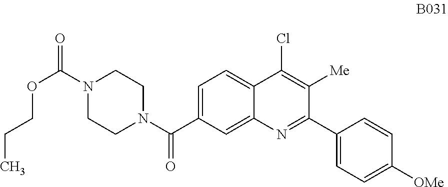 [SMILES: CCCOC(=O)N1CCN(C(=O)c2ccc3c(Cl)c(C)c(-c4ccc(OC)cc4)nc3c2)CC1]

Compound B031 was synthesized according to General Procedure I, K and C1 using 4-methoxypropiophenone (General Procedure I) and n-propyl piperazine-1-carboxylate (General Procedure C1) as starting materials.

¹H NMR (400 MHz, DMSO-d₆) δ 8.25 (d, J=8.6 Hz, 1H), 8.02 (s, 1H), 7.70 (d, J=8.6 Hz, 1H), 7.57 (d, J=8.5 Hz, 2H), 7.08 (d, J=8.5 Hz, 2H), 3.99 (t, J=6.5 Hz, 2H), 3.85 (s, 3H), 3.75-3.42 (m, 8H), 2.54 (s, 3H), 1.64-1.51 (m, 2H), 0.88 (dd, J=13.2, 5.9 Hz, 3H).

LCMS (ESI-TOF) m/z 482.1 [M+H⁺] with a purity of >99%.

### Propyl 4-(4-chloro-2-(3-cyanophenyl)-3-methylquinoline-7-carbonyl)piperazine-1-carboxylate (B032)

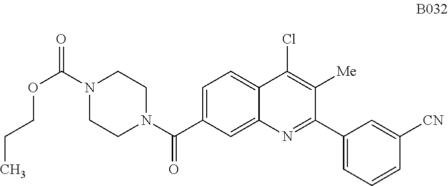 [SMILES: CCCOC(=O)N1CCN(C(=O)c2ccc3c(Cl)c(C)c(-c4cccc(C#N)c4)nc3c2)CC1]

Compound B032 was synthesized according to General Procedure I, K and C1 using 3-cyanopropiophenone (General Procedure I) and n-propyl piperazine-1-carboxylate (General Procedure C1) as starting materials.

¹H NMR (400 MHz, DMSO-d₆) δ 8.30 (d, J=8.6 Hz, 1H), 8.07 (s, 2H), 7.95 (t, J=6.5 Hz, 2H), 7.79-7.68 (m, 2H), 3.99 (t, J=6.6 Hz, 2H), 3.50 (d, J=30.3 Hz, 8H), 2.51 (s, 3H), 1.65-1.51 (m, 2H), 0.89 (t, J=7.4 Hz, 3H).

LCMS (ESI-TOF) m/z 477.1 [M+H⁺] with a purity of >99%.

### Propyl 4-(2-(3-carbamoylphenyl)-4-chloroquinoline-7-carbonyl)piperazine-1-carboxylate (B033)

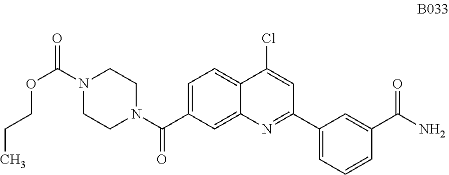 [SMILES: CCCOC(=O)N1CCN(C(=O)c2ccc3c(Cl)cc(-c4cccc(C(N)=O)c4)nc3c2)CC1]

Compound B033 was synthesized according to General Procedure I, K and C1 using 3-acetylbenzonitrile (General Procedure I) and n-propyl piperazine-1-carboxylate (General Procedure C1) as starting materials. B033 was a side-product obtained from General Procedure K.

¹H NMR (400 MHz, DMSO-d₆) δ 8.77 (s, 1H), 8.57 (s, 1H), 8.49 (d, J=7.8 Hz, 1H), 8.31 (d, J=8.6 Hz, 1H), 8.20-8.13 (m, 2H), 8.04 (d, J=7.8 Hz, 1H), 7.77 (dd, J=8.6, 1.6 Hz, 1H), 7.67 (t, J=7.7 Hz, 1H), 7.53 (s, 1H), 3.98 (t, J=6.6 Hz, 2H), 3.81-3.33 (m, 8H), 1.65-1.50 (m, 2H), 0.88 (dd, J=14.6, 7.5 Hz, 3H).

LCMS (ESI-TOF) m/z 481.1 [M+H⁺] with a purity of >99%.

### Propyl (S)-4-(4-chloro-2-(4-methoxyphenyl)-3-methylquinoline-7-carbonyl)-2-methylpiperazine-1-carboxylate (B034)

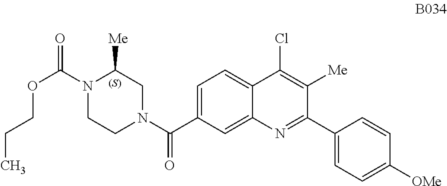

Compound B034 was synthesized according to General Procedure I, K and C1 using 4-methoxypropiophenone (General Procedure I) and (S)-n-propyl 2-methylpiperazine-1-carboxylate (General Procedure C1) as starting materials.

¹H NMR (400 MHz, DMSO-d₆) δ 8.26 (d, J=8.5 Hz, 1H), 8.00 (s, 1H), 7.70 (d, J=8.5 Hz, 1H), 7.58 (d, J=8.4 Hz, 2H), 7.08 (d, J=8.4 Hz, 2H), 4.41-3.69 (m, 8H), 3.37-3.08 (m, 4H), 2.54 (s, 3H), 1.70-1.48 (m, 2H), 1.12 (d, J=6.4 Hz, 3H), 0.97-0.76 (m, 3H).

LCMS (ESI-TOF) m/z 496.2 [M+H⁺] with a purity of >98%.

### Propyl 4-(2-(3-carbamoylphenyl)-4-chloro-3-methylquinoline-7-carbonyl)piperazine-1-carboxylate (B035)

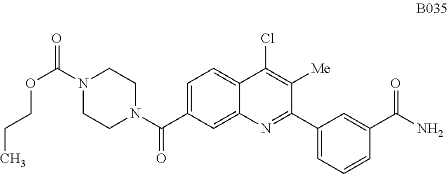 [SMILES: CCCOC(=O)N1CCN(C(=O)c2ccc3c(Cl)c(C)c(-c4cccc(C(N)=O)c4)nc3c2)CC1]

Compound B035 was synthesized according to General Procedure I, K and C1 using 3-propionylbenzonitrile (General Procedure I) and n-propyl piperazine-1-carboxylate (General Procedure C1) as starting materials. B035 was a side-product obtained from General Procedure K.

¹H NMR (400 MHz, DMSO-d₆) δ 8.30 (d, J=8.6 Hz, 1H), 8.12 (s, 1H), 8.09 (d, J=1.3 Hz, 1H), 8.07 (br s, 1H), 8.02 (d, J=7.9 Hz, 1H), 7.82-7.72 (m, 2H), 7.63 (t, J=7.7 Hz, 1H), 7.47 (br s, 1H), 3.97 (t, J=6.6 Hz, 2H), 3.78-3.36 (m, 8H), 2.52 (s, 3H), 1.63-1.52 (m, 2H), 0.89 (t, J=7.2 Hz, 3H).

LCMS (ESI-TOF) m/z 495.1 [M+H⁺] with a purity of >96%.

### Propyl (R)-4-(4-chloro-2-(4-methoxyphenyl)-3-methylquinoline-7-carbonyl)-2-methylpiperazine-1-carboxylate (B036)

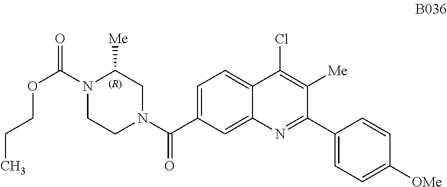

Compound B036 was synthesized according to General Procedure I, K and C1 using 4-methoxypropiophenone (General Procedure I) and (R)-n-propyl 2-methylpiperazine-1-carboxylate (General Procedure C1) as starting materials.

¹H NMR (400 MHz, DMSO-d₆) δ 8.28 (d, J=8.6 Hz, 1H), 8.02 (s, 1H), 7.72 (d, J=8.5 Hz, 1H), 7.60 (d, J=8.5 Hz, 2H), 7.10 (d, J=8.6 Hz, 2H), 4.33-3.72 (m, 8H), 3.41-3.07 (m, 4H), 2.56 (s, 3H), 1.67-1.49 (m, 2H), 1.14 (d, J=6.4 Hz, 3H), 0.91 (t, J=7.4 Hz, 3H).

LCMS (ESI-TOF) m/z 496.2 [M+H⁺] with a purity of >97%.

### Propyl 4-(4-chloro-2-(4-cyanophenyl)-3-methylquinoline-7-carbonyl)piperazine-1-carboxylate (B037)

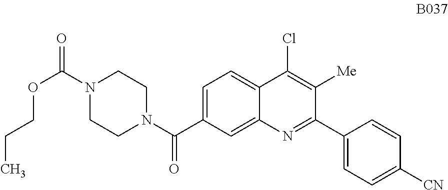 [SMILES: CCCOC(=O)N1CCN(C(=O)c2ccc3c(Cl)c(C)c(-c4ccc(C#N)cc4)nc3c2)CC1]

Compound B037 was synthesized according to General Procedure I, K and C1 using 4-propionylbenzonitrile (General Procedure I) and n-propyl piperazine-1-carboxylate (General Procedure C1) as starting materials.

¹H NMR (400 MHz, DMSO-d₆) δ 8.31 (d, J=8.6 Hz, 1H), 8.09 (d, J=1.0 Hz, 1H), 8.03 (d, J=8.3 Hz, 2H), 7.85 (d, J=8.4 Hz, 2H), 7.79 (dd, J=9.7, 2.6 Hz, 1H), 3.97 (t, J=6.5 Hz, 2H), 3.76-3.46 (m, 8H), 1.64-1.52 (m, 2H), 0.89 (t, J=7.3 Hz, 3H).

LCMS (ESI-TOF) m/z 477.1 [M+H⁺] with a purity of >99%.

### Propyl 4-(2-(3-(aminomethyl)phenyl)-4-chloro-3-methylquinoline-7-carbonyl)piperazine-1-carboxylate (B038)

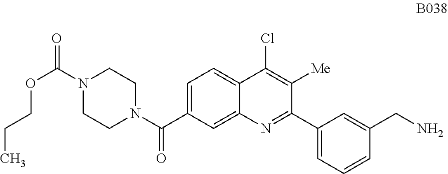 [SMILES: CCCOC(=O)N1CCN(C(=O)c2ccc3c(Cl)c(C)c(-c4cccc(CN)c4)nc3c2)CC1]

Compound B038 was synthesized according to General Procedure I, K, C1 and D using 3-propionylbenzonitrile (General Procedure I) and n-propyl piperazine-1-carboxylate (General Procedure C1) as starting materials.

¹H NMR (400 MHz, 80° C., DMSO-d₆) δ 8.27 (d, J=8.5 Hz, 1H), 8.04 (s, 1H), 7.73 (d, J=8.7 Hz, 1H), 7.55 (s, 1H), 7.49-7.38 (m, 3H), 3.99 (t, J=6.4 Hz, 2H), 3.83 (s, 2H), 3.50 (d, J=30.8 Hz, 8H), 2.50 (d, J=3.9 Hz, 3H), 1.65-1.51 (m, 2H), 0.89 (t, J=7.3 Hz, 3H).

LCMS (ESI-TOF) m/z 481.2 [M+H⁺] with a purity of >99%.

### Propyl 4-(2-(3-(aminomethyl)phenyl)-4-chloroquinoline-7-carbonyl)piperazine-1-carboxylate (B039)

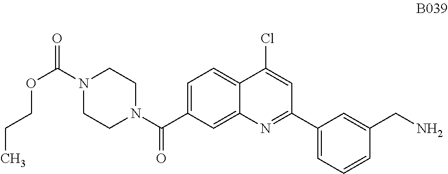 [SMILES: CCCOC(=O)N1CCN(C(=O)c2ccc3c(Cl)cc(-c4cccc(CN)c4)nc3c2)CC1]

Compound B039 was synthesized according to General Procedure I, K, C1 and D using 3-acetylbenzonitrile (General Procedure I) and n-propyl piperazine-1-carboxylate (General Procedure C1) as starting materials.

¹H NMR (400 MHz, 80° C., DMSO-d₆) δ 8.43-8.36 (m, 1H), 8.26 (t, J=8.0 Hz, 2H), 8.13 (s, 2H), 7.72 (d, J=8.5 Hz, 1H), 7.55-7.45 (m, 2H), 3.99 (t, J=6.4 Hz, 2H), 3.86 (s, 2H), 3.62-3.40 (m, 8H), 1.62-1.53 (m, 2H), 0.90 (t, J=7.5 Hz, 3H).

LCMS (ESI-TOF) m/z 467.2 [M+H⁺] with a purity of >98%.

### Propyl 4-(4-chloro-2-(phenylamino)quinoline-7-carbonyl)piperazine-1-carboxylate (B040)

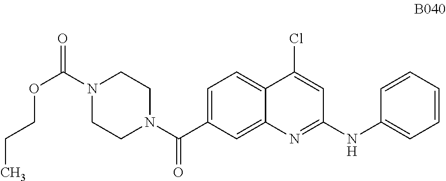 [SMILES: CCCOC(=O)N1CCN(C(=O)c2ccc3c(Cl)cc(Nc4ccccc4)nc3c2)CC1]

Step 1: Phosphorus oxychloride (1.9 mL) was added to malonic acid (378.6 mg, 3.64 mmol, 1.1 equiv) at 0° C. After 10 min, methyl 3-aminobenzoate (500 mg, 3.308 mmol) was added at the same temperature, before warming up to room temperature. The mixture was heated to reflux for 4 h before cooling it to room temperature. The contents were emptied into 2M aqueous sodium hydroxide and diluted with dichloromethane. At pH 7, the aqueous layer was extracted 4 times with dichloromethane, and the combined organic layers were washed with water and then brine. Upon drying over sodium sulfate, the mixture was filtered and concentrated. The crude material was purified by column chromatography to afford methyl 2,4-dichloroquinoline-7-carboxylate (112.2 mg, 13%).

Step 2: To a previously dried reaction vessel was added the above intermediate (28.5 mg, 0.11 mmol), aniline (25 mg, 0.139 mmol, 1.26 equiv), caesium carbonate (72 mg, 0.22 mmol, 2 equiv), XantPhos (19 mg, 0.0328 mmol, 0.3 equiv), tris(dibenzylideneacetone)dipalladium(0) (10 mg, 0.0109 mmol, 0.1 equiv) and previously degassed 1,4-dioxane (1 mL). The resulting mixture was heated at 110° C. for 2 h before filtering the contents with dichloromethane. The concentrated crude material was purified by column chromatography to afford methyl 4-chloro-2-(phenylamino)quinoline-7-carboxylate (30 mg, 87%).

Step 3: Methyl 4-chloro-2-(phenylamino)quinoline-7-carboxylate was hydrolysed according to General Procedure K to afford 4-chloro-2-(phenylamino)quinoline-7-carboxylic acid.

Step 4: Compound B040 was synthesized according to General Procedure C1 using the above intermediate and n-propyl piperazine-1-carboxylate as reagents.

¹H NMR (400 MHz, DMSO-d₆) δ 9.64 (s, 1H), 8.03 (d, J=8.4 Hz, 1H), 7.94 (d, J=7.7 Hz, 2H), 7.73 (d, J=1.3 Hz, 1H), 7.40 (dd, J=8.3, 1.5 Hz, 1H), 7.38-7.33 (m, 2H), 7.31 (s, 1H), 7.01 (t, J=7.3 Hz, 1H), 3.97 (t, J=6.6 Hz, 2H), 3.75-3.33 (m, 8H), 1.58 (dd, J=13.8, 7.1 Hz, 2H), 0.89 (t, J=7.3 Hz, 3H).

LCMS (ESI-TOF) m/z 453.1 [M+H⁺] with a purity of >97%.

### Propyl 4-(4-chloro-2-((4-methoxyphenyl)amino)quinoline-7-carbonyl)piperazine-1-carboxylate (B041)

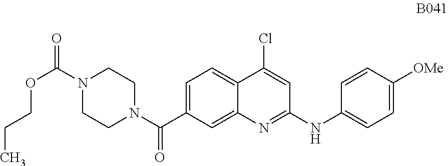 [SMILES: CCCOC(=O)N1CCN(C(=O)c2ccc3c(Cl)cc(Nc4ccc(OC)cc4)nc3c2)CC1]

Step 1: To a previously dried reaction vessel was added the above intermediate methyl 2,4-dichloroquinoline-7-carboxylate (30 mg, 0.12 mmol), 4-methoxyaniline (18 mg, 0.1 mmol, 0.83 equiv), caesium carbonate (78 mg, 0.239 mmol, 2 equiv), XantPhos (20 mg, 0.0346 mmol, 0.29 equiv), tris(dibenzylideneacetone)dipalladium(0) (11 mg, 0.012 mmol, 0.1 equiv) and previously degassed 1,4-dioxane (1.5 mL). The resulting mixture was heated at 110° C. for 2 h before filtering the contents with dichloromethane. The concentrated crude material was purified by column chromatography to afford methyl 4-chloro-2-((4-methoxyphenyl)amino)quinoline-7-carboxylate (10 mg, 27%).

Step 2: Methyl 4-chloro-2-((4-methoxyphenyl)amino)quinoline-7-carboxylate was hydrolysed according to General Procedure K to afford 4-chloro-2-((4-methoxyphenyl)amino)quinoline-7-carboxylic acid.

Step 3: Compound B041 was synthesized according to General Procedure C1 using the above intermediate and n-propyl piperazine-1-carboxylate as reagents.

¹H NMR (400 MHz, DMSO-d₆) δ 9.48 (s, 1H), 7.99 (d, J=8.3 Hz, 1H), 7.82 (d, J=9.0 Hz, 2H), 7.66 (d, J=1.3 Hz, 1H), 7.36 (dd, J=8.3, 1.5 Hz, 1H), 7.23 (s, 1H), 6.94 (d, J=9.1 Hz, 2H), 3.97 (t, J=6.6 Hz, 2H), 3.75 (s, 3H), 3.71-3.35 (m, 8H), 1.65-1.51 (m, 2H), 0.89 (t, J=7.3 Hz, 3H).

LCMS (ESI-TOF) m/z 483.1 [M+H⁺] with a purity of >96%.

### Propyl 4-(4-chloro-2-(pyridin-3-ylamino)quinoline-7-carbonyl)piperazine-1-carboxylate (B042)

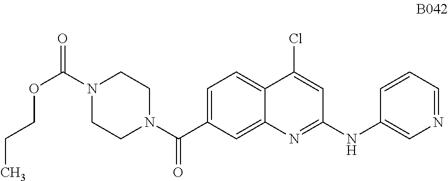 [SMILES: CCCOC(=O)N1CCN(C(=O)c2ccc3c(Cl)cc(Nc4cccnc4)nc3c2)CC1]

Step 1: To a previously dried reaction vessel was added the above intermediate methyl 2,4-dichloroquinoline-7-carboxylate (30 mg, 0.12 mmol), 3-aminopyridine (14 mg, 0.144 mmol, 1.2 equiv), caesium carbonate (78 mg, 0.239 mmol, 2 equiv), XantPhos (20 mg, 0.0346 mmol, 0.29 equiv), tris(dibenzylideneacetone)dipalladium(0) (11 mg, 0.012 mmol, 0.1 equiv) and previously degassed 1,4-dioxane (1.5 mL). The resulting mixture was heated at 110° C. for 2 h before filtering the contents with dichloromethane. The concentrated crude material was purified by column chromatography to afford methyl 4-chloro-2-(pyridin-3-ylamino)quinoline-7-carboxylate (17 mg, 45%).

Step 2: Methyl 4-chloro-2-(pyridin-3-ylamino)quinoline-7-carboxylate was hydrolysed according to General Procedure K to afford 4-chloro-2-(pyridin-3-ylamino)quinoline-7-carboxylic acid.

Step 3: Compound B042 was synthesized according to General Procedure C1 using the above intermediate and n-propyl piperazine-1-carboxylate as reagents.

¹H NMR (400 MHz, DMSO-d₆) δ 9.87 (s, 1H), 9.01 (d, J=2.4 Hz, 1H), 8.55-8.47 (m, 1H), 8.22 (dd, J=4.6, 1.3 Hz, 1H), 8.06 (d, J=8.4 Hz, 1H), 7.79 (d, J=1.2 Hz, 1H), 7.44 (dd, J=8.4, 1.5 Hz, 1H), 7.38 (dd, J=8.3, 4.7 Hz, 1H), 7.34 (s, 1H), 3.97 (t, J=6.6 Hz, 2H), 3.80-3.34 (m, 8H), 1.64-1.51 (m, 2H), 0.89 (t, J=7.3 Hz, 3H).

LCMS (ESI-TOF) m/z 454.1 [M+H⁺] with a purity of >99%.

### Propyl 4-(4-chloro-3-methyl-2-(3-nitrophenyl)quinoline-7-carbonyl)piperazine-1-carboxylate (B043)

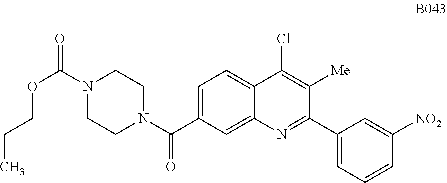 [SMILES: CCCOC(=O)N1CCN(C(=O)c2ccc3c(Cl)c(C)c(-c4cccc([N+](=O)[O-])c4)nc3c2)CC1]

Compound B043 was synthesized according to General Procedure I, K and C1 using 3-nitropropiophenone (General Procedure I) and n-propyl piperazine-1-carboxylate (General Procedure C1) as starting materials.

¹H NMR (400 MHz, DMSO-d₆) δ 8.49-8.46 (m, 1H), 8.38 (dd, J=8.2, 2.3 Hz, 1H), 8.32 (d, J=8.6 Hz, 1H), 8.16-8.10 (m, 2H), 7.85 (t, J=8.0 Hz, 1H), 7.80 (dd, J=8.6, 1.5 Hz, 1H), 3.97 (t, J=6.6 Hz, 2H), 3.81-3.32 (m, 8H), 2.54 (s, 3H), 1.67-1.51 (m, 2H), 0.89 (t, J=7.3 Hz, 3H).

LCMS (ESI-TOF) m/z 497.1 [M+H⁺] with a purity of >97%.

### Propyl 4-(2-(3-aminophenyl)-4-chloro-3-methylquinoline-7-carbonyl)piperazine-1-carboxylate (B044)

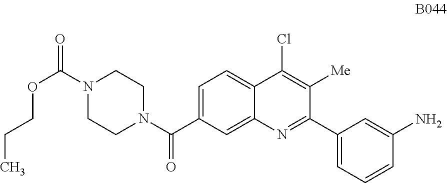 [SMILES: CCCOC(=O)N1CCN(C(=O)c2ccc3c(Cl)c(C)c(-c4cccc(N)c4)nc3c2)CC1]

Compound B043 (39 mg, 0.08 mmol) was dissolved in ethanol (2 mL) and iron powder (27 mg, 0.48 mmol, 6 equiv) was added. Solid ammonium chloride (47 mg, 0.879 mmol, 11 equiv) was dissolved in water (0.5 mL) and the solution was added to the slurry. The slurry was heated at 70° C. for 15 min and then diluted with methanol and filtered upon cooling. The concentrated crude material was purified by preparative HPLC to afford compound B044 as an off-white solid upon lyophilization (6 mg, 16%).

¹H NMR (400 MHz, DMSO-d₆) δ 8.25 (d, J=8.6 Hz, 1H), 8.01 (s, 1H), 7.71 (d, J=8.7 Hz, 1H), 7.15 (t, J=7.7 Hz, 1H), 6.77 (s, 1H), 6.69 (t, J=7.0 Hz, 2H), 5.04 (s, 2H), 3.99 (t, J=6.5 Hz, 2H), 3.50 (d, J=31.7 Hz, 8H), 1.66-1.52 (m, 2H), 0.89 (t, J=7.4 Hz, 3H).

LCMS (ESI-TOF) m/z 467.1 [M+H⁺] with a purity of >95%.

### Propyl (R)-4-(4-chloro-2-(4-methoxyphenyl)quinoline-7-carbonyl)-2-methylpiperazine-1-carboxylate (B045)

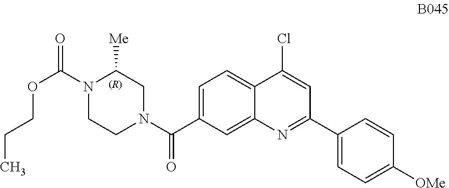

Compound B045 was synthesized according to General Procedure I, K and C1 using 4-methoxyacetophenone (General Procedure I) and (R)-n-propyl 2-methylpiperazine-1-carboxylate (General Procedure C1) as starting materials.

¹H NMR (400 MHz, 80° C., DMSO-d₆) δ 8.33 (s, 1H), 8.25 (t, J=8.5 Hz, 3H), 8.06 (s, 1H), 7.68 (d, J=8.7 Hz, 1H), 7.11 (d, J=8.9 Hz, 2H), 4.37-3.74 (m, 10H), 3.41-3.21 (m, 2H), 1.60 (dt, J=13.9, 6.9 Hz, 2H), 1.14 (d, J=5.6 Hz, 3H), 0.89 (t, J=7.4 Hz, 3H).

LCMS (ESI-TOF) m/z 482.1 [M+H⁺] with a purity of >99%.

### Propyl 4-(4-chloro-2-(4-methoxyphenyl)quinoline-7-carbonyl)piperazine-1-carboxylate (B046)

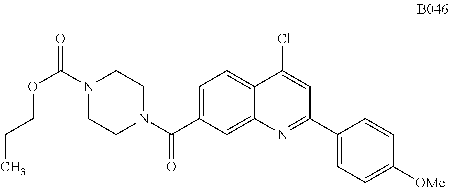 [SMILES: CCCOC(=O)N1CCN(C(=O)c2ccc3c(Cl)cc(-c4ccc(OC)cc4)nc3c2)CC1]

Compound B045 was synthesized according to General Procedure I, K and C1 using 4-methoxyacetophenone (General Procedure I) and n-propyl piperazine-1-carboxylate (General Procedure C1) as starting materials.

¹H NMR (400 MHz, 80° C., DMSO-d₆) δ 8.32 (s, 1H), 8.25 (t, J=9.3 Hz, 3H), 8.08 (s, 1H), 7.68 (d, J=8.2 Hz, 1H), 7.11 (d, J=8.5 Hz, 2H), 3.99 (t, J=6.6 Hz, 2H), 3.86 (s, 3H), 3.51 (d, J=30.8 Hz, 8H), 1.58 (dt, J=28.7, 14.4 Hz, 2H), 0.90 (t, J=7.5 Hz, 3H).

LCMS (ESI-TOF) m/z 468.2 [M+H⁺] with a purity of >99%.

### Propyl (S)-4-(4-chloro-2-(4-methoxyphenyl)quinoline-7-carbonyl)-3-methylpiperazine-1-carboxylate (B047)

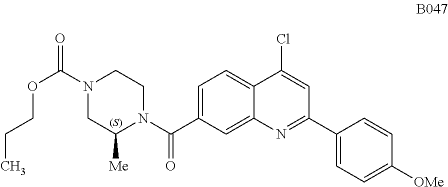

Compound B047 was synthesized according to General Procedure I, K and C1 using 4-methoxyacetophenone (General Procedure I) and (S)-n-propyl 3-methylpiperazine-1-carboxylate (General Procedure C1) as starting materials.

¹H NMR (400 MHz, DMSO-d₆) δ 8.33 (s, 1H), 8.30-8.21 (m, 3H), 8.04 (s, 1H), 7.66 (d, J=8.5 Hz, 1H), 7.11 (d, J=8.9 Hz, 2H), 4.57-3.68 (m, 10H), 3.19 (d, J=7.6 Hz, 2H), 1.59 (dd, J=14.2, 7.1 Hz, 2H), 1.20 (d, J=6.8 Hz, 3H), 0.90 (t, J=7.4 Hz, 3H).

LCMS (ESI-TOF) m/z 482.1 [M+H⁺] with a purity of >98%.

### Propyl 4-(4-chloro-2-(3,5-difluorophenyl)quinoline-7-carbonyl)piperazine-1-carboxylate (B048)

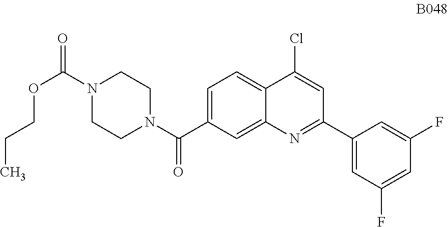 [SMILES: CCCOC(=O)N1CCN(C(=O)c2ccc3c(Cl)cc(-c4cc(F)cc(F)c4)nc3c2)CC1]

Compound B048 was synthesized according to General Procedure I, K and C1 using 3,5-difluoroacetophenone (General Procedure I) and n-propyl piperazine-1-carboxylate (General Procedure C2) as reagents.

¹H NMR (400 MHz, DMSO-d₆) δ 8.61 (s, 1H), 8.30 (d, J=8.6 Hz, 1H), 8.16 (d, J=23.4 Hz, 1H), 8.09 (d, J=7.0 Hz, 1H), 7.80 (d, J=8.5 Hz, 1H), 7.45 (t, J=9.0 Hz, 1H), 3.98 (t, J=6.5 Hz, 2H), 3.83-3.33 (m, 8H), 1.59 (dd, J=13.8, 6.9 Hz, 2H), 0.89 (t, J=7.1 Hz, 3H).

LCMS (ESI-TOF) m/z 474.1 [M+H⁺] with a purity of >98%.

### Propyl 4-(4-chloro-2-(3-fluorophenyl)quinoline-7-carbonyl)piperazine-1-carboxylate (B049)

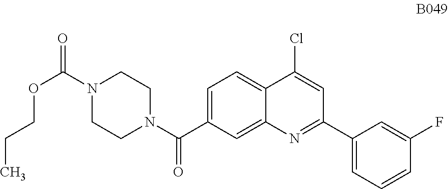 [SMILES: CCCOC(=O)N1CCN(C(=O)c2ccc3c(Cl)cc(-c4cccc(F)c4)nc3c2)CC1]

Compound B049 was synthesized according to General Procedure I, K and C1 using 3-fluoroacetophenone (General Procedure I) and n-propyl piperazine-1-carboxylate (General Procedure C1) as starting materials.

¹H NMR (400 MHz, 80° C., DMSO-d₆) δ 8.44 (s, 1H), 8.29 (d, J=8.5 Hz, 1H), 8.20-8.04 (m, 3H), 7.75 (dd, J=8.5, 1.4 Hz, 1H), 7.60 (dd, J=14.1, 8.0 Hz, 1H), 7.35 (td, J=8.2, 2.0 Hz, 1H), 3.99 (t, J=6.6 Hz, 2H), 3.52 (d, J=30.8 Hz, 8H), 1.68-1.52 (m, 2H), 0.90 (t, J=7.4 Hz, 3H).

LCMS (ESI-TOF) m/z 456.1 [M+H⁺] with a purity of >99%.

### Propyl 4-(4-chloro-2-(4-(pyrrolidin-1-yl)phenyl)quinoline-7-carbonyl)piperazine-1-carboxylate (B050)

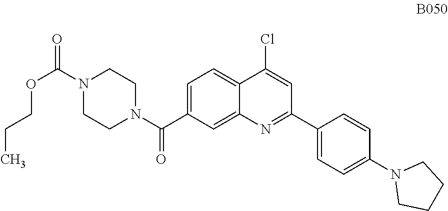 [SMILES: CCCOC(=O)N1CCN(C(=O)c2ccc3c(Cl)cc(-c4ccc(N5CCCC5)cc4)nc3c2)CC1]

Compound B050 was synthesized according to General Procedure I, K and C1 using 4′-(1-pyrrolidino)acetophenone (General Procedure I) and n-propyl piperazine-1-carboxylate (General Procedure C1) as starting materials.

¹H NMR (400 MHz, 80° C., DMSO-d₆) δ 8.22 (s, 1H), 8.16 (dd, J=8.5, 6.9 Hz, 3H), 7.99 (s, 1H), 7.59 (d, J=9.9 Hz, 1H), 6.68 (d, J=8.8 Hz, 2H), 3.99 (t, J=6.6 Hz, 2H), 3.51 (d, J=31.8 Hz, 8H), 3.34 (d, J=6.4 Hz, 4H), 2.00 (t, J=6.4 Hz, 4H), 1.60 (dd, J=14.1, 6.9 Hz, 2H), 0.90 (t, J=7.4 Hz, 3H).

LCMS (ESI-TOF) m/z 507.2 [M+H⁺] with a purity of >96%.

### Propyl 4-(4-chloroquinoline-7-carbonyl)piperazine-1-carboxylate (B051)

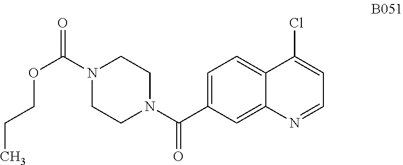 [SMILES: CCCOC(=O)N1CCN(C(=O)c2ccc3c(Cl)ccnc3c2)CC1]

Compound B051 was synthesized via General Procedure C2 using n-propyl piperazine-1-carboxylate and 4-chloroquinoline-6-carboxylic acid as starting materials.

¹H NMR (400 MHz, DMSO-d₆) δ 8.92 (d, J=4.7 Hz, 1H), 8.29 (d, J=8.6 Hz, 1H), 8.12 (s, 1H), 7.86 (d, J=4.7 Hz, 1H), 7.79 (d, J=8.6 Hz, 1H), 3.98 (t, J=6.6 Hz, 2H), 3.79-3.37 (m, 8H), 1.72-1.45 (m, 2H), 0.89 (t, J=7.3 Hz, 3H).

LCMS (ESI-TOF) m/z 362.1 [M+H⁺] with a purity of >96%.

### Propyl (R)-4-(4-chloro-2-(p-tolyl)quinoline-7-carbonyl)-2-methylpiperazine-1-carboxylate (B052)

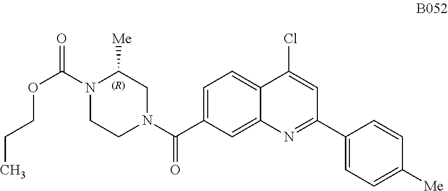

Compound B052 was synthesized according to General Procedure I, K and C2 using 4-methylacetophenone (General Procedure I) and (R)-n-propyl 2-methylpiperazine (General Procedure C2) as reagents.

¹H NMR (400 MHz, 80° C., DMSO-d₆) δ 8.35 (s, 1H), 8.26 (d, J=8.5 Hz, 1H), 8.19 (d, J=8.2 Hz, 2H), 8.09 (d, J=1.1 Hz, 1H), 7.70 (dd, J=8.5, 1.5 Hz, 1H), 7.37 (d, J=8.0 Hz, 2H), 4.37-3.71 (m, 6H), 3.44-3.10 (m, 3H), 2.40 (s, 3H), 1.72-1.47 (m, 2H), 1.14 (d, J=6.2 Hz, 3H), 0.89 (t, J=7.4 Hz, 3H).

LCMS (ESI-TOF) m/z 466.1 [M+H⁺] with a purity of >97%.

### Propyl (R)-4-(2-(3-carbamoylphenyl)-4-chloroquinoline-7-carbonyl)-2-methylpiperazine-1-carboxylate (B053)

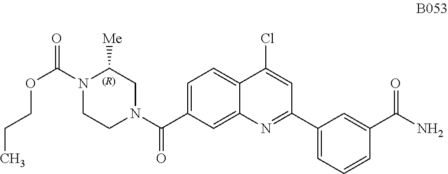

Compound B053 was synthesized according to General Procedure I, K and C1 using 3-acetylbenzonitrile (General Procedure I) and (R)-n-propyl 2-methylpiperazine-1-carboxylate (General Procedure C1) as starting materials. B053 was a side-product obtained from General Procedure K.

¹H NMR (400 MHz, 80° C., DMSO-d₆) δ 8.73 (s, 1H), 8.47 (s, 1H), 8.45 (d, J=7.9 Hz, 1H), 8.30 (d, J=8.5 Hz, 1H), 8.15 (s, 1H), 8.03 (d, J=7.7 Hz, 1H), 7.77-7.73 (m, 1H), 7.64 (t, J=7.7 Hz, 1H), 4.40-3.71 (m, 6H), 3.38-3.10 (m, 3H), 1.67-1.50 (m, 2H), 1.14 (d, J=6.2 Hz, 3H), 0.89 (t, J=7.4 Hz, 3H).

LCMS (ESI-TOF) m/z 495.1 [M+H⁺] with a purity of >96%.

### Propyl (R)-4-(2-(3-(aminomethyl)phenyl)-4-chloroquinoline-7-carbonyl)-2-methylpiperazine-1-carboxylate (B054)

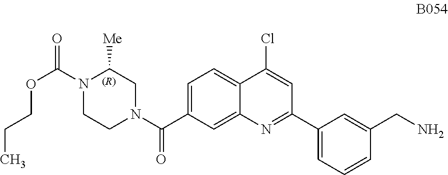

Compound B054 was synthesized according to General Procedure I, K, C1 and D using 3-acetylbenzonitrile (General Procedure I) and (R)-n-propyl 2-methylpiperazine-1-carboxylate (General Procedure C1) as starting materials.

¹H NMR (400 MHz, 80° C., DMSO-d₆) δ 8.38 (s, 1H), 8.30-8.22 (m, 2H), 8.15-8.07 (m, 2H), 7.72 (dd, J=8.5, 1.5 Hz, 1H), 7.53-7.44 (m, 2H), 4.34-3.74 (m, 7H), 3.43-3.05 (m, 4H), 1.66-1.51 (m, 2H), 1.14 (d, J=6.4 Hz, 3H), 0.89 (t, J=7.4 Hz, 3H).

LCMS (ESI-TOF) m/z 481.2 [M+H⁺] with a purity of >99%.

### Propyl 4-(4-chloro-2-(4-(dimethylamino)phenyl)quinoline-7-carbonyl)piperazine-1-carboxylate (B055)

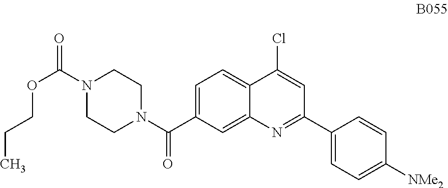 [SMILES: CCCOC(=O)N1CCN(C(=O)c2ccc3c(Cl)cc(-c4ccc(N(C)C)cc4)nc3c2)CC1]

Compound B055 was synthesized according to General Procedure I, K and C1 using 4′-dimethylaminoacetophenone (General Procedure I) and n-propyl piperazine-1-carboxylate (General Procedure C1) as starting materials.

¹H NMR (400 MHz, DMSO-d₆) δ 8.34 (s, 1H), 8.20 (dd, J=8.6, 4.7 Hz, 3H), 8.01 (s, 1H), 7.63 (d, J=8.5 Hz, 1H), 6.84 (d, J=9.0 Hz, 2H), 3.98 (t, J=6.5 Hz, 2H), 3.79-3.35 (m, 8H), 3.03 (s, 6H), 1.67-1.50 (m, 2H), 0.89 (t, J=7.3 Hz, 3H).

LCMS (ESI-TOF) m/z 481.2 [M+H⁺] with a purity of >95%.

### Propyl (R)-4-(2-(3-aminophenyl)-4-chloro-3-methylquinoline-7-carbonyl)-2-methylpiperazine-1-carboxylate (B056)

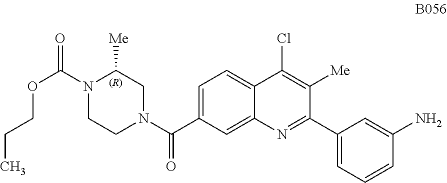

Step 1: Intermediate 2 for compound B043 was subjected to General Procedure C1 with (R)-n-propyl 2-methylpiperazine-1-carboxylate to afford propyl (R)-4-(4-chloro-3-methyl-2-(3-nitrophenyl)quinoline-7-carbonyl)-2-methylpiperazine-1-carboxylate.

Step 2: Intermediate from Step 1 (387 mg, 0.78 mmol) was dissolved in ethanol (20 mL) and iron powder (263 mg, 4.7 mmol, 6 equiv) was added. Solid ammonium chloride (460.1 mg, 8.6 mmol, 11 equiv) was dissolved in water (5 mL) and the solution was added to the slurry. The slurry was heated at 70° C. for 15 min and then diluted with methanol and filtered upon cooling. The concentrated crude material was purified by preparative HPLC to afford compound B056 as an off-white solid upon lyophilization (221 mg, 59%).

¹H NMR (400 MHz, 80° C., DMSO-d₆) δ 8.26 (d, J=8.6 Hz, 1H), 7.99 (d, J=1.1 Hz, 1H), 7.70 (dd, J=8.6, 1.5 Hz, 1H), 7.15 (t, J=7.8 Hz, 1H), 6.77 (d, J=1.7 Hz, 1H), 6.70 (dd, J=9.8, 4.8 Hz, 2H), 5.03 (s, 2H), 4.33-3.71 (m, 6H), 3.43-3.09 (m, 3H), 2.50 (s, 3H), 1.65-1.46 (m, 2H), 1.09 (t, J=16.2 Hz, 3H), 0.89 (t, J=7.4 Hz, 3H).

LCMS (ESI-TOF) m/z 481.2 [M+H⁺] with a purity of >98%.

### Propyl 4-(4-chloro-2-(4-ethynylphenyl)quinoline-7-carbonyl)piperazine-1-carboxylate (B057)

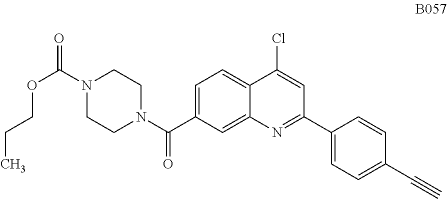 [SMILES: C#Cc1ccc(-c2cc(Cl)c3ccc(C(=O)N4CCN(C(=O)OCCC)CC4)cc3n2)cc1]

Compound B057 was synthesized according to General Procedure I, K and C1 using 4-acetylphenylacetylene (General Procedure I) and n-propyl piperazine-1-carboxylate (General Procedure C1) as starting materials.

¹H NMR (400 MHz, DMSO-d₆) δ 8.53 (s, 1H), 8.36 (d, J=8.4 Hz, 2H), 8.29 (d, J=8.5 Hz, 1H), 8.15 (s, 1H), 7.77 (dd, J=8.5, 1.4 Hz, 1H), 7.68 (d, J=8.4 Hz, 2H), 4.39 (s, 1H), 3.98 (t, J=6.5 Hz, 2H), 3.82-3.35 (m, 8H), 1.63-1.52 (m, 2H), 0.89 (t, J=7.3 Hz, 3H).

LCMS (ESI-TOF) m/z 462.1 [M+H⁺] with a purity of >99%.

### Propyl 4-(4-chloro-2-(3-(dimethylamino)phenyl)quinoline-7-carbonyl)piperazine-1-carboxylate (B058)

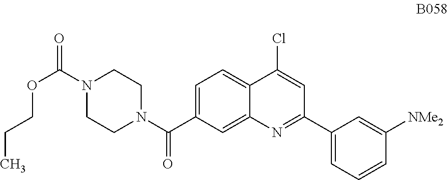 [SMILES: CCCOC(=O)N1CCN(C(=O)c2ccc3c(Cl)cc(-c4cccc(N(C)C)c4)nc3c2)CC1]

Compound B058 was synthesized according to General Procedure I, K and C1 using 3′-dimethylaminoacetophenone (General Procedure I) and n-propyl piperazine-1-carboxylate (General Procedure C1) as starting materials.

¹H NMR (400 MHz, DMSO-d₆) δ 8.45 (s, 1H), 8.27 (d, J=8.5 Hz, 1H), 8.14 (s, 1H), 7.73 (dd, J=8.5, 1.4 Hz, 1H), 7.62 (s, 1H), 7.59 (d, J=7.7 Hz, 1H), 7.37 (t, J=7.9 Hz, 1H), 6.91 (dd, J=8.2, 2.3 Hz, 1H), 3.98 (t, J=6.6 Hz, 2H), 3.82-3.34 (m, 8H), 3.02 (s, 6H), 1.63-1.51 (m, 2H), 0.89 (t, J=7.3 Hz, 3H).

LCMS (ESI-TOF) m/z 481.2 [M+H⁺] with a purity of >99%.

### Propyl (R)-4-(4-chloro-2-(4-ethynylphenyl)quinoline-7-carbonyl)-2-methylpiperazine-1-carboxylate (B059)

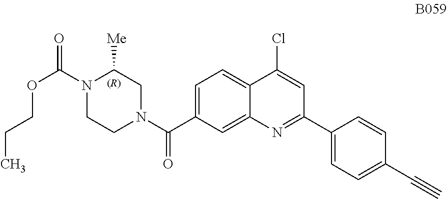

Compound B059 was synthesized according to General Procedure I, K and C1 using 4-acetylphenylacetylene (General Procedure I) and (R)-n-propyl 2-methylpiperazine-1-carboxylate (General Procedure C1) as starting materials.

¹H NMR (400 MHz, 80° C., DMSO-d₆) δ 8.42 (s, 1H), 8.32 (d, J=8.4 Hz, 2H), 8.29 (d, J=8.5 Hz, 1H), 8.12 (d, J=0.9 Hz, 1H), 7.74 (dd, J=8.5, 1.4 Hz, 1H), 7.65 (d, J=8.4 Hz, 2H), 4.36-3.68 (m, 7H), 3.42-3.09 (m, 3H), 1.67-1.52 (m, 2H), 1.14 (d, J=6.2 Hz, 3H), 0.89 (t, J=7.4 Hz, 3H).

LCMS (ESI-TOF) m/z 476.1 [M+H⁺] with a purity of >96%.

### Propyl (R)-4-(4-chloro-2-(4-(dimethylamino)phenyl)quinoline-7-carbonyl)-2-methylpiperazine-1-carboxylate (B060)

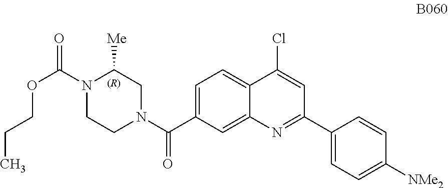

Compound B060 was synthesized according to General Procedure I, K and C1 using 4′-dimethylaminoacetophenone (General Procedure I) and (R)-n-propyl 2-methylpiperazine-1-carboxylate (General Procedure C1) as starting materials.

¹H NMR (400 MHz, 80° C., DMSO-d₆) δ 8.23 (s, 1H), 8.22-8.10 (m, 3H), 7.99 (s, 1H), 7.60 (d, J=8.5 Hz, 1H), 6.84 (d, J=9.0 Hz, 2H), 4.46-3.65 (m, 6H), 3.37-3.09 (m, 3H), 3.02 (s, 6H), 1.59 (dt, J=13.9, 7.1 Hz, 2H), 1.14 (d, J=6.3 Hz, 3H), 0.89 (t, J=7.4 Hz, 3H).

LCMS (ESI-TOF) m/z 495.2 [M+H⁺] with a purity of >98%.

### Propyl (R)-4-(4-chloro-2-(3-(dimethylamino)phenyl)quinoline-7-carbonyl)-2-methylpiperazine-1-carboxylate (B061)

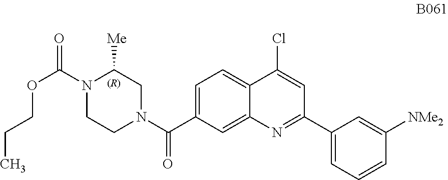

Compound B061 was synthesized according to General Procedure I, K and C1 using 3′-dimethylaminoacetophenone (General Procedure I) and (R)-n-propyl 2-methylpiperazine-1-carboxylate (General Procedure C1) as starting materials.

¹H NMR (400 MHz, 80° C., DMSO-d₆) δ 8.34 (s, 1H), 8.26 (d, J=8.5 Hz, 1H), 8.10 (s, 1H), 7.70 (dd, J=8.5, 1.5 Hz, 1H), 7.60 (s, 1H), 7.54 (d, J=7.7 Hz, 1H), 7.35 (t, J=7.9 Hz, 1H), 6.90 (dd, J=8.3, 2.2 Hz, 1H), 4.38-3.70 (m, 6H), 3.42-3.11 (m, 3H), 3.01 (s, 6H), 1.66-1.54 (m, 2H), 1.14 (d, J=6.2 Hz, 3H), 0.89 (t, J=7.4 Hz, 3H).

LCMS (ESI-TOF) m/z 495.2 [M+H⁺] with a purity of >98%.

### Propyl (R)-4-(4-chloro-2-(4-cyanophenyl)quinoline-7-carbonyl)-2-methylpiperazine-1-carboxylate (B062)

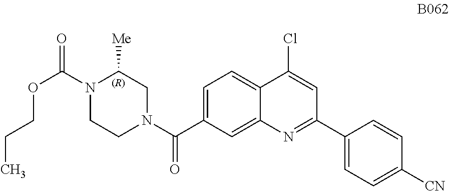

Compound B062 was synthesized according to General Procedure I, K and C1 using 4-acetylbenzonitrile (General Procedure I) and (R)-n-propyl 2-methylpiperazine-1-carboxylate (General Procedure C1) as starting materials.

¹H NMR (400 MHz, 80° C., DMSO-d₆) δ 8.49 (d, J=8.8 Hz, 3H), 8.31 (d, J=8.6 Hz, 1H), 8.15 (d, J=1.0 Hz, 1H), 8.00 (d, J=8.4 Hz, 2H), 7.78 (dd, J=8.5, 1.5 Hz, 1H), 4.50-3.56 (m, 6H), 3.43-3.09 (m, 3H), 1.67-1.52 (m, 2H), 1.14 (d, J=6.1 Hz, 3H), 0.89 (t, J=7.4 Hz, 3H).

LCMS (ESI-TOF) m/z 477.1 [M+H⁺] with a purity of >95%.

### Propyl (R)-4-(2-(4-carbamoylphenyl)-4-chloroquinoline-7-carbonyl)-2-methylpiperazine-1-carboxylate (B063)

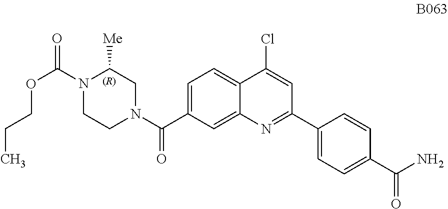

Compound B063 was synthesized according to General Procedure I, K and C1 using 4-acetylbenzonitrile (General Procedure I) and (R)-n-propyl 2-methylpiperazine-1-carboxylate (General Procedure C1) as starting materials. Compound B063 is a side-product in the synthesis of B062.

¹H NMR (400 MHz, 80° C., DMSO-d₆) δ 8.45 (s, 1H), 8.36 (d, J=7.9 Hz, 2H), 8.30 (d, J=8.6 Hz, 1H), 8.14 (s, 1H), 8.05 (d, J=7.9 Hz, 2H), 7.75 (d, J=8.5 Hz, 1H), 4.46-3.67 (m, 6H), 3.47-3.10 (m, 3H), 1.59 (dt, J=14.1, 7.1 Hz, 2H), 1.14 (d, J=6.2 Hz, 3H), 0.90 (t, J=7.4 Hz, 3H).

LCMS (ESI-TOF) m/z 495.1 [M+H⁺] with a purity of >95%.

### Propyl (R)-4-(2-(4-(aminomethyl)phenyl)-4-chloroquinoline-7-carbonyl)-2-methylpiperazine-1-carboxylate (B064)

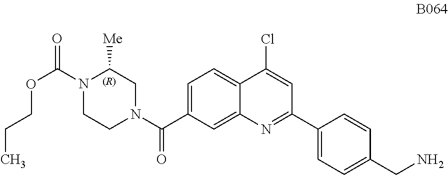

Compound B064 was synthesized from B062 using General Procedure D.

¹H NMR (400 MHz, 80° C., DMSO-d₆) δ 8.36 (s, 1H), 8.27 (d, J=8.5 Hz, 1H), 8.23 (d, J=8.3 Hz, 2H), 8.09 (s, 1H), 7.74-7.62 (m, 1H), 7.52 (d, J=8.2 Hz, 2H), 4.36-3.74 (m, 8H), 3.39-3.14 (m, 3H), 1.67-1.53 (m, 2H), 1.14 (d, J=6.4 Hz, 3H), 0.89 (t, J=7.4 Hz, 3H).

LCMS (ESI-TOF) m/z 481.2 [M+H⁺] with a purity of >97%.

### Propyl 4-(4-chloro-2-(4-fluorophenyl)quinoline-7-carbonyl)piperazine-1-carboxylate (B065)

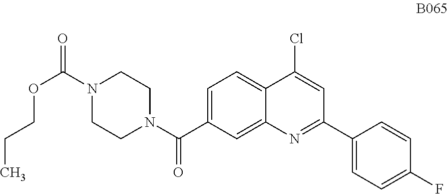 [SMILES: CCCOC(=O)N1CCN(C(=O)c2ccc3c(Cl)cc(-c4ccc(F)cc4)nc3c2)CC1]

Compound B065 was synthesized according to General Procedure I, K and C1 using 4-fluoroacetophenone (General Procedure I) and n-propyl piperazine-1-carboxylate as starting materials.

¹H NMR (400 MHz, 80° C., DMSO-d₆) δ 8.39 (s, 1H), 8.36 (dd, J=8.8, 5.6 Hz, 2H), 8.27 (d, J=8.5 Hz, 1H), 8.12 (s, 1H), 7.73 (dd, J=8.5, 1.4 Hz, 1H), 7.36 (t, J=8.8 Hz, 2H), 3.99 (t, J=6.6 Hz, 2H), 3.51 (d, J=30.7 Hz, 8H), 1.67-1.51 (m, 2H), 0.96-0.80 (m, 3H).

LCMS (ESI-TOF) m/z 456.1 [M+H⁺] with a purity of >99%.

### Propyl 4-(4-chloro-2-(4-(trifluoromethyl)phenyl)quinoline-7-carbonyl)piperazine-1-carboxylate (B066)

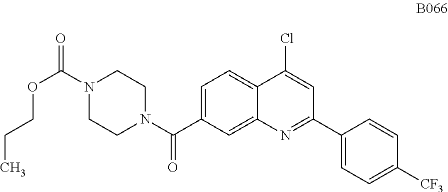 [SMILES: CCCOC(=O)N1CCN(C(=O)c2ccc3c(Cl)cc(-c4ccc(C(F)(F)F)cc4)nc3c2)CC1]

Compound B066 was synthesized according to General Procedure I, K and C1 using 4-trifluoroacetophenone (General Procedure I) and n-propyl piperazine-1-carboxylate as starting materials.

¹H NMR (400 MHz, 80° C., DMSO-d₆) δ 8.51 (d, J=8.1 Hz, 2H), 8.48 (s, 1H), 8.31 (d, J=8.5 Hz, 1H), 8.17 (s, 1H), 7.90 (d, J=8.2 Hz, 2H), 7.78 (d, J=8.5 Hz, 1H), 4.00 (t, J=6.5 Hz, 2H), 3.52 (d, J=31.9 Hz, 8H), 1.68-1.51 (m, 2H), 0.90 (t, J=7.4 Hz, 3H).

LCMS (ESI-TOF) m/z 506.1 [M+H⁺] with a purity of >95%.

### Propyl 4-(4-chloro-2-(3-ethylphenyl)quinoline-7-carbonyl)piperazine-1-carboxylate (B067)

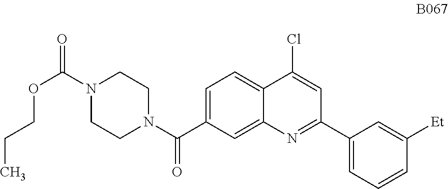 [SMILES: CCCOC(=O)N1CCN(C(=O)c2ccc3c(Cl)cc(-c4cccc(CC)c4)nc3c2)CC1]

Compound B067 was synthesized according to General Procedure I, K and C1 using 3-ethylacetophenone (General Procedure I) and n-propyl piperazine-1-carboxylate as starting materials.

¹H NMR (400 MHz, DMSO-d₆) δ 8.48 (s, 1H), 8.28 (d, J=8.5 Hz, 1H), 8.15 (t, J=10.8 Hz, 3H), 7.74 (dd, J=8.5, 1.4 Hz, 1H), 7.49 (t, J=7.6 Hz, 1H), 7.40 (d, J=7.6 Hz, 1H), 3.98 (t, J=6.6 Hz, 2H), 3.83-3.34 (m, 8H), 2.75 (q, J=7.6 Hz, 2H), 1.59 (dd, J=13.9, 6.9 Hz, 2H), 1.28 (t, J=7.6 Hz, 3H), 0.89 (t, J=7.3 Hz, 3H).

LCMS (ESI-TOF) m/z 466.2 [M+H⁺] with a purity of >99%.

### Propyl 4-(4-chloro-2-(4-fluoro-3-methylphenyl)quinoline-7-carbonyl)piperazine-1-carboxylate (B068)

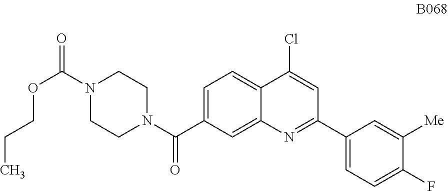 [SMILES: CCCOC(=O)N1CCN(C(=O)c2ccc3c(Cl)cc(-c4ccc(F)c(C)c4)nc3c2)CC1]

Compound B068 was synthesized according to General Procedure I, K and C1 using 4-fluoro-3-methylacetophenone (General Procedure I) and n-propyl piperazine-1-carboxylate as starting materials.

¹H NMR (400 MHz, DMSO-d₆) δ 8.48 (s, 1H), 8.29 (t, J=8.4 Hz, 2H), 8.24-8.17 (m, 1H), 8.13 (d, J=0.9 Hz, 1H), 7.74 (dd, J=8.5, 1.5 Hz, 1H), 7.33 (t, J=9.1 Hz, 1H), 3.98 (t, J=6.6 Hz, 2H), 3.74-3.34 (m, 8H), 2.37 (s, 3H), 1.59 (dd, J=13.8, 6.7 Hz, 3H), 0.89 (t, J=7.3 Hz, 3H).

LCMS (ESI-TOF) m/z 470.1 [M+H⁺] with a purity of >96%.

### Propyl (R)-4-(4-chloro-2-(3-morphohnophenyl)quinoline-7-carbonyl)-2-methylpiperazine-1-carboxylate (B069)

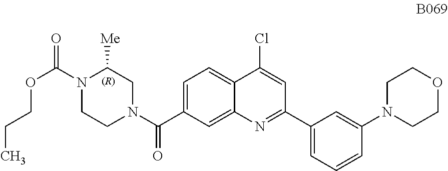

Compound B069 was synthesized according to General Procedure I, K and C1 using 1-(3-morpholin-4-yl-phenyl)ethanone (General Procedure I) and (R)-n-propyl 2-methylpiperazine-1-carboxylate as starting materials.

¹H NMR (400 MHz, 80° C., DMSO-d₆) δ 8.39 (s, 1H), 8.27 (d, J=8.5 Hz, 1H), 8.11 (s, 1H), 7.82 (s, 1H), 7.76-7.67 (m, 2H), 7.41 (t, J=7.9 Hz, 1H), 7.10 (d, J=8.0 Hz, 1H), 4.42-3.70 (m, 10H), 3.40-3.13 (m, 7H), 1.65-1.54 (m, 2H), 1.13 (s, 3H), 0.89 (t, J=7.4 Hz, 3H).

LCMS (ESI-TOF) m/z 537.2 [M+H⁺] with a purity of >95%.

### Propyl 4-(4-chloro-2-(3-ethynylphenyl)quinoline-7-carbonyl)piperazine-1-carboxylate (B070)

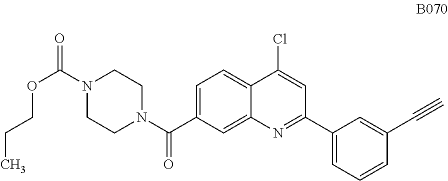 [SMILES: C#Cc1cccc(-c2cc(Cl)c3ccc(C(=O)N4CCN(C(=O)OCCC)CC4)cc3n2)c1]

Compound B070 was synthesized according to General Procedure I, K and C1 using 3-acetylphenylacetylene (General Procedure I) and n-propyl piperazine-1-carboxylate (General Procedure C1) as starting materials.

¹H NMR (400 MHz, DMSO-d₆) δ 8.56 (s, 1H), 8.45 (s, 1H), 8.37 (d, J=7.5 Hz, 1H), 8.29 (d, J=8.3 Hz, 1H), 8.18 (s, 1H), 7.77 (d, J=7.9 Hz, 1H), 7.70-7.53 (m, 2H), 4.31 (s, 1H), 3.98 (t, J=6.2 Hz, 2H), 3.81-3.36 (m, 8H), 1.59 (dd, J=11.0, 4.8 Hz, 2H), 0.89 (t, J=6.1 Hz, 3H).

LCMS (ESI-TOF) m/z 462.1 [M+H⁺] with a purity of >99%.

### Propyl (R)-4-(4-chloro-2-(3-ethynylphenyl)quinoline-7-carbonyl)-2-methylpiperazine-1-carboxylate (B071)

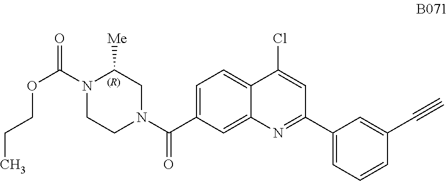

Compound B071 was synthesized according to General Procedure I, K and C1 using 3-acetylphenylacetylene (General Procedure I) and (R)-n-propyl 2-methylpiperazine-1-carboxylate (General Procedure C1) as starting materials.

¹H NMR (400 MHz, 80° C., DMSO-d₆) δ 8.46 (s, 1H), 8.41 (s, 1H), 8.33 (d, J=7.7 Hz, 1H), 8.29 (d, J=8.6 Hz, 1H), 8.14 (s, 1H), 7.74 (dd, J=8.5, 1.4 Hz, 1H), 7.60 (dt, J=15.3, 7.6 Hz, 2H), 4.35-3.70 (m, 7H), 3.41-3.10 (m, 3H), 1.68-1.52 (m, 2H), 1.14 (d, J=5.3 Hz, 3H), 0.93-0.85 (m, 3H).

LCMS (ESI-TOF) m/z 476.1 [M+H⁺] with a purity of >99%.

### Propyl 4-(4-chloro-2-(4-nitrophenyl)quinoline-7-carbonyl)piperazine-1-carboxylate (B072)

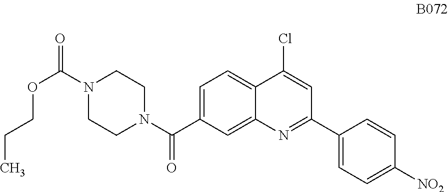 [SMILES: CCCOC(=O)N1CCN(C(=O)c2ccc3c(Cl)cc(-c4ccc([N+](=O)[O-])cc4)nc3c2)CC1]

Compound B072 was synthesized according to General Procedure I, K and C1 using 4-nitroacetophenone (General Procedure I) and n-propyl piperazine-1-carboxylate (General Procedure C1) as starting materials.

¹H NMR (400 MHz, 80° C., DMSO-d₆) δ 8.56 (d, J=8.9 Hz, 2H), 8.53 (s, 1H), 8.38 (d, J=8.9 Hz, 2H), 8.32 (d, J=8.6 Hz, 1H), 8.19 (d, J=0.9 Hz, 1H), 7.80 (dd, J=8.5, 1.5 Hz, 1H), 4.00 (t, J=6.6 Hz, 2H), 3.67-3.38 (m, 8H), 1.68-1.47 (m, 2H), 0.90 (t, J=7.4 Hz, 3H).

LCMS (ESI-TOF) m/z 483.1 [M+H⁺] with a purity of >97%.

### Propyl 4-(2-(4-aminophenyl)-4-chloroquinoline-7-carbonyl)piperazine-1-carboxylate (B073)

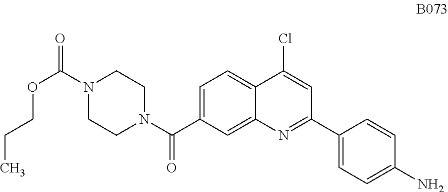 [SMILES: CCCOC(=O)N1CCN(C(=O)c2ccc3c(Cl)cc(-c4ccc(N)cc4)nc3c2)CC1]

Compound B072 (45 mg, 0.093 mmol) was dissolved in ethanol (5 mL) and iron powder (45 mg, 0.806 mmol, 8.6 equiv) was added. Solid ammonium chloride (45 mg, 0.841 mmol, 9 equiv) was dissolved in water (5 mL) and the solution was added to the slurry. The slurry was heated at 80° C. for 15 min and then diluted with methanol and filtered upon cooling. The concentrated crude material was purified by column chromatography to afford compound B073 as a yellow solid upon lyophilization (25 mg, 59%).

¹H NMR (400 MHz, 80° C., DMSO-d₆) δ 8.21-8.14 (m, 2H), 8.02 (d, J=8.6 Hz, 2H), 7.98 (s, 1H), 7.60 (d, J=8.5 Hz, 1H), 6.71 (d, J=8.6 Hz, 2H), 5.45 (s, 2H), 3.99 (t, J=6.5 Hz, 2H), 3.62-3.40 (m, 8H), 1.66-1.52 (m, 2H), 0.90 (t, J=7.4 Hz, 3H).

LCMS (ESI-TOF) m/z 453.1 [M+H⁺] with a purity of >99%.

### Propyl (R)-4-(2-(4-aminophenyl)-4-chloroquinoline-7-carbonyl)-2-methylpiperazine-1-carboxylate (B074)

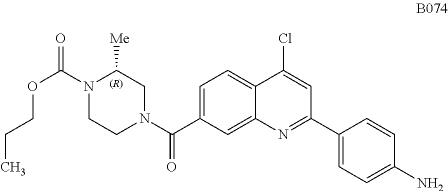

Step 1: Intermediate 2 for compound B072 was subjected to General Procedure C1 with (R)-n-propyl 2-methylpiperazine-1-carboxylate to afford propyl (R)-4-(4-chloro-2-(4-nitrophenyl)quinoline-7-carbonyl)-2-methylpiperazine-1-carboxylate.

Step 2: Intermediate from Step 1 (70 mg, 0.141 mmol) was dissolved in ethanol (5 mL) and iron powder (70 mg, 1.25 mmol, 8.9 equiv) was added. Solid ammonium chloride (70 mg, 1.31 mmol, 9.3 equiv) was dissolved in water (5 mL) and the solution was added to the slurry. The slurry was heated at 80° C. for 15 min and then diluted with methanol and filtered upon cooling. The concentrated crude material was purified by column chromatography to afford compound B074 as a yellow solid upon lyophilization (36.2 mg, 55%).

¹H NMR (400 MHz, 80° C., DMSO-d₆) δ 8.27-8.08 (m, 2H), 8.02 (d, J=8.5 Hz, 2H), 7.96 (s, 1H), 7.59 (d, J=8.5 Hz, 1H), 6.71 (d, J=8.6 Hz, 2H), 5.46 (s, 2H), 4.46-3.62 (m, 6H), 3.36-3.10 (m, 3H), 1.67-1.52 (m, 2H), 1.13 (d, J=6.4 Hz, 3H), 0.89 (t, J=7.4 Hz, 3H).

LCMS (ESI-TOF) m/z 467.1 [M+H⁺] with a purity of >99%.

### Propyl (R)-4-(4-chloro-2-(4-(pyrrolidin-1-yl)phenyl)quinoline-7-carbonyl)-2-methylpiperazine-1-carboxylate (B075)

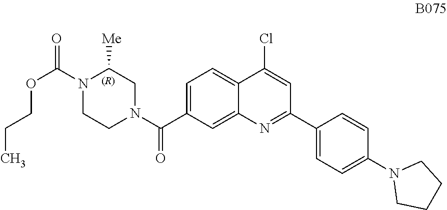

Compound B075 was synthesized according to General Procedure I, K and C1 using 4′-(1-pyrrolidino)acetophenone (General Procedure I) and (R)-n-propyl 2-methylpiperazine-1-carboxylate (General Procedure C1) as starting materials.

¹H NMR (400 MHz, 80° C., DMSO-d₆) δ 8.25-8.10 (m, 4H), 7.97 (d, J=1.0 Hz, 1H), 7.59 (dd, J=8.5, 1.4 Hz, 1H), 6.68 (d, J=8.9 Hz, 2H), 4.35-3.68 (m, 6H), 3.35 (t, J=6.5 Hz, 4H), 3.32-3.08 (m, 3H), 2.00 (t, J=6.5 Hz, 4H), 1.66-1.54 (m, 2H), 1.13 (d, J=6.4 Hz, 3H), 0.89 (t, J=7.4 Hz, 3H).

LCMS (ESI-TOF) m/z 521.2 [M+H⁺] with a purity of >99%.

### Propyl (S)-4-(4-chloro-2-(4-(dimethylamino)phenyl)quinoline-7-carbonyl)-3-methylpiperazine-1-carboxylate (B076)

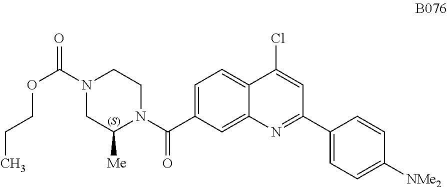

Compound B076 was synthesized according to General Procedure I, K and C1 using 4′-dimethylaminoacetophenone (General Procedure I) and (S)-n-propyl 3-methylpiperazine-1-carboxylate (General Procedure C1) as starting materials.

¹H NMR (400 MHz, 80° C., DMSO-d₆) δ 8.27-8.11 (m, 4H), 7.97 (d, J=1.1 Hz, 1H), 7.58 (dd, J=8.5, 1.6 Hz, 1H), 6.84 (d, J=8.6 Hz, 2H), 4.49-3.69 (m, 6H), 3.32-3.14 (m, 2H), 3.02 (s, 6H), 3.02-2.92 (m, 1H), 1.67-1.53 (m, 2H), 1.20 (d, J=6.8 Hz, 3H), 0.90 (t, J=7.4 Hz, 3H).

LCMS (ESI-TOF) m/z 495.2 [M+H⁺] with a purity of >95%.

### Propyl 4-(2-(3-aminophenyl)-4-chloroquinoline-7-carbonyl)piperazine-1-carboxylate (B077)

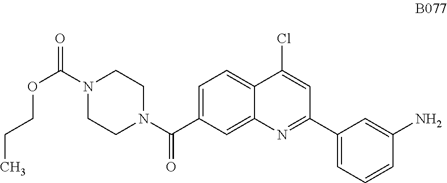 [SMILES: CCCOC(=O)N1CCN(C(=O)c2ccc3c(Cl)cc(-c4cccc(N)c4)nc3c2)CC1]

Step 1-3: Propyl 4-(4-chloro-2-(3-nitrophenyl)quinoline-7-carbonyl)piperazine-1-carboxylate was synthesized via General Procedure I, K and C1 using 3-nitroacetophenone (General Procedure I) and n-propyl piperazine-1-carboxylate (General Procedure C1) as starting materials.

Step 4: Intermediate from above (200 mg, 0.414 mmol) was dissolved in ethanol (6 mL) and iron powder (138.77 mg, 2.485 mmol, 6 equiv) was added. Solid ammonium chloride (243.7 mg, 4.56 mmol, 11 equiv) was dissolved in water (5 mL) and the solution was added to the slurry. The slurry was heated at 70° C. for 15 min and then diluted with methanol and filtered upon cooling. The concentrated crude material was purified by column chromatography to afford compound B077 as an off-white solid upon lyophilization (180 mg, 96%).

¹H NMR (400 MHz, DMSO-d₆) δ 8.27 (s, 1H), 8.26 (d, J=6.3 Hz, 1H), 8.09 (s, 1H), 7.73 (d, J=8.5 Hz, 1H), 7.52 (s, 1H), 7.40 (d, J=7.6 Hz, 1H), 7.20 (t, J=7.8 Hz, 1H), 6.74 (d, J=7.5 Hz, 1H), 5.29 (s, 2H), 3.98 (t, J=6.5 Hz, 2H), 3.82-3.33 (m, 8H), 1.59 (dd, J=13.9, 6.9 Hz, 2H), 0.89 (t, J=7.2 Hz, 3H).

LCMS (ESI-TOF) m/z 453.1 [M+H⁺] with a purity of >98%.

### Propyl (R)-4-(2-(3-aminophenyl)-4-chloroquinoline-7-carbonyl)-2-methylpiperazine-1-carboxylate (B078)

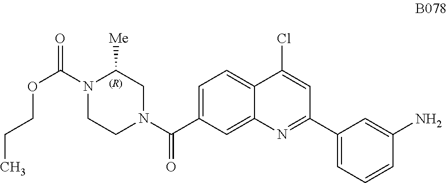

Step 1-3: Propyl (R)-4-(4-chloro-2-(3-nitrophenyl)quinoline-7-carbonyl)-2-methylpiperazine-1-carboxylate was synthesized via General Procedure I, K and C1 using 3-nitroacetophenone (General Procedure I) and (R)-n-propyl 2-methylpiperazine-1-carboxylate (General Procedure C1) as starting materials.

Step 4: Intermediate from above (300 mg, 0.604 mmol) was dissolved in ethanol (8 mL) and iron powder (202.3 mg, 3.622 mmol, 6 equiv) was added. Solid ammonium chloride (355.3 mg, 6.64 mmol, 11 equiv) was dissolved in water (5 mL) and the solution was added to the slurry. The slurry was heated at 70° C. for 15 min and then diluted with methanol and filtered upon cooling. The concentrated crude material was purified by column chromatography to afford compound B078 as an off-white solid upon lyophilization (180 mg, 64%).

¹H NMR (400 MHz, 80° C., DMSO-d₆) δ 8.26 (d, J=8.6 Hz, 1H), 8.20 (s, 1H), 8.06 (s, 1H), 7.70 (d, J=8.6 Hz, 1H), 7.52 (s, 1H), 7.39 (d, J=7.6 Hz, 1H), 7.20 (t, J=7.8 Hz, 1H), 6.75 (d, J=7.4 Hz, 1H), 5.08 (s, 2H), 4.35-3.69 (m, 6H), 3.41-3.10 (m, 3H), 1.69-1.52 (m, 2H), 1.13 (d, J=5.9 Hz, 3H), 0.89 (t, J=7.4 Hz, 3H).

LCMS (ESI-TOF) m/z 467.1 [M+H⁺] with a purity of >98%.

### Propyl (R)-4-(4-chloro-2-cyclopropylquinoline-7-carbonyl)-2-methylpiperazine-1-carboxylate (B079)

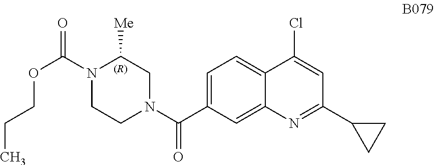

Compound B079 was synthesized via General Procedure L using propyl (R)-4-(2,4-dichloroquinoline-7-carbonyl)-2-methylpiperazine-1-carboxylate and cyclopropyl boronic acid as starting material.

¹H NMR (400 MHz, 80° C., DMSO-d₆) δ 8.17 (d, J=8.5 Hz, 1H), 7.87 (d, J=1.1 Hz, 1H), 7.71 (s, 1H), 7.60 (dd, J=8.5, 1.5 Hz, 1H), 4.35-3.68 (m, 6H), 3.31-3.09 (m, 3H), 2.43-2.26 (m, 1H), 1.70-1.51 (m, 2H), 1.20-1.03 (m, 7H), 0.93-0.85 (m, 3H).

LCMS (ESI-TOF) m/z 416.1 [M+H⁺] with a purity of >98%.

### Propyl (R)-4-(4-chloro-2-(thiazol-2-yl)quinoline-7-carbonyl)-2-methylpiperazine-1-carboxylate (B080)

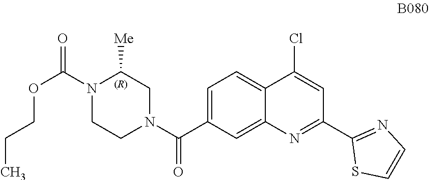

Compound B080 was synthesized according to General Procedure I, K and C1 using 2-acetylthiazole (General Procedure I) and (R)-n-propyl 2-methylpiperazine-1-carboxylate (General Procedure C1) as starting materials.

¹H NMR (400 MHz, 80° C., DMSO-d₆) δ 8.44 (s, 1H), 8.32 (d, J=8.6 Hz, 1H), 8.11 (d, J=0.9 Hz, 1H), 8.09 (d, J=3.1 Hz, 1H), 7.97 (d, J=3.1 Hz, 1H), 7.78 (dd, J=8.6, 1.5 Hz, 1H), 4.39-3.67 (m, 6H), 3.52-3.11 (m, 3H), 1.69-1.50 (m, 2H), 1.14 (d, J=6.1 Hz, 3H), 0.89 (t, J=7.4 Hz, 3H).

LCMS (ESI-TOF) m/z 459.0 [M+H⁺] with a purity of >99%.

### Propyl 3-(4-chloro-2-phenylquinoline-7-carbonyl)-3,9-diazabicyclo[3.3.1]nonane-9-carboxylate (B081)

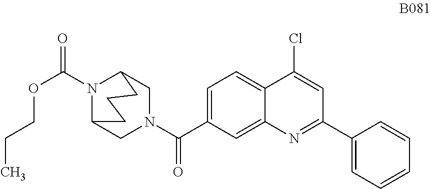 [SMILES: CCCOC(=O)N1C2CCCC1CN(C(=O)c1ccc3c(Cl)cc(-c4ccccc4)nc3c1)C2]

Step 1: Intermediate from General Procedure K in the synthesis of B002 was subjected to General Procedure C1 with tert-butyl 3,9-diazabicyclo[3.3.1]nonane-9-carboxylate as reagent to afford tert-butyl 3-(4-chloro-2-phenylquinoline-7-carbonyl)-3,9-diazabicyclo [3.3.1]nonane-9-carboxylate.

Step 2: Intermediate from Step 1 (118.5 mg, 0.2408 mmol) was dissolved in dichloromethane (0.2 mL) and trifluoroacetic acid was added (0.2 mL). After 20 min, the mixture was concentrated and re-dissolved in ethyl acetate. The organic layer was washed with saturated bicarbonate, dried over anhydrous sodium sulfate, filtered and concentrated to give crude (3,9-diazabicyclo[3.3.1]nonan-3-yl)(4-chloro-2-phenylquinolin-7-yl)methanone.

Step 3: The crude intermediate from above (77.7 mg, 0.1982 mmol) was dissolved in dichloromethane (2 mL) and triethylamine (60 μL, 0.43 mmol, 2.2 equiv) and propyl chloroformate (30 μL, 0.258 mmol, 1.3 equiv) were added. After 30 min, the mixture was quenched by adding saturated ammonium chloride, followed by extraction with dichloromethane. The combined organic layer was dried over anhydrous sodium sulfate, filtered and concentrated. The crude material was purified by column chromatography to afford B081 (27.7 mg, 29%) as a white solid upon lyophilization.

¹H NMR (400 MHz, 80° C., DMSO-d₆) δ 8.38 (s, 1H), 8.32-8.24 (m, 3H), 8.07 (d, J=1.1 Hz, 1H), 7.69 (dd, J=8.5, 1.6 Hz, 1H), 7.63-7.48 (m, 3H), 4.52-3.82 (m, 5H), 3.54-3.15 (m, 2H), 2.20-2.04 (m, 1H), 2.00-1.50 (m, 8H), 0.90 (t, J=7.4 Hz, 3H).

LCMS (ESI-TOF) m/z 478.1 [M+H⁺] with a purity of >98%.

### Propyl (R)-4-(4-chloro-2-(4-(dimethylcarbamoyl)phenyl)quinoline-7-carbonyl)-2-methylpiperazine-1-carboxylate (B082)

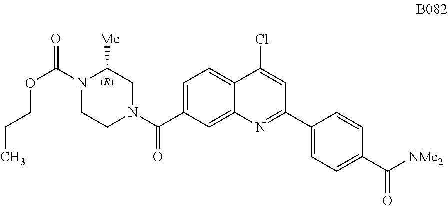

Compound B082 was synthesized according to General Procedure I, K and C1 using 4-acetyl-N,N-dimethylbenzamide (General Procedure I) and (R)-n-propyl 2-methylpiperazine-1-carboxylate (General Procedure C1) as starting materials.

¹H NMR (400 MHz, 80° C., DMSO-d₆) δ 8.42 (d, J=8.1 Hz, 1H), 8.35 (d, J=8.3 Hz, 2H), 8.29 (d, J=8.5 Hz, 1H), 8.13 (s, 1H), 7.74 (dd, J=8.5, 1.4 Hz, 1H), 7.58 (d, J=8.3 Hz, 2H), 4.40-3.73 (m, 6H), 3.40-3.10 (m, 3H), 2.99 (s, 6H), 1.68-1.54 (m, 2H), 1.14 (d, J=6.3 Hz, 3H), 0.89 (t, J=7.4 Hz, 3H).

LCMS (ESI-TOF) m/z 523.2 [M+H⁺] with a purity of >97%.

### Propyl (R)-4-(4-chloro-2-(3-(methylamino)phenyl)quinoline-7-carbonyl)-2-methylpiperazine-1-carboxylate (B083)

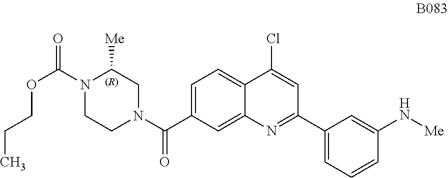

Compound B083 was synthesized according to General Procedure I, K and C1 using 1-(3-(methylamino)phenyl)ethanone (General Procedure I) and (R)-n-propyl 2-methylpiperazine-1-carboxylate (General Procedure C1) as starting materials.

¹H NMR (400 MHz, 80° C., DMSO-d₆) δ 8.26 (d, J=8.7 Hz, 1H), 8.25 (s, 1H), 8.08 (s, 1H), 7.70 (dd, J=8.5, 1.5 Hz, 1H), 7.49-7.36 (m, 2H), 7.26 (t, J=7.8 Hz, 1H), 6.72 (d, J=8.0 Hz, 1H), 5.61 (d, J=5.1 Hz, 1H), 4.34-3.75 (m, 6H), 3.39-3.09 (m, 3H), 2.80 (d, J=5.1 Hz, 3H), 1.67-1.49 (m, 2H), 1.14 (d, J=6.4 Hz, 3H), 0.89 (t, J=7.4 Hz, 3H).

LCMS (ESI-TOF) m/z 481.1 [M+H⁺] with a purity of >96%.

### Propyl 4-(4-chloro-2-(thiophen-2-yl)quinoline-7-carbonyl)piperazine-1-carboxylate (B084)

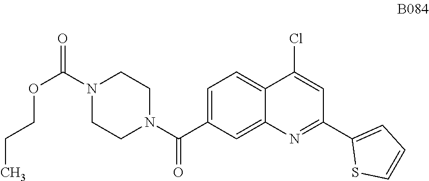 [SMILES: CCCOC(=O)N1CCN(C(=O)c2ccc3c(Cl)cc(-c4cccs4)nc3c2)CC1]

Compound B084 was synthesized according to General Procedure I, K and C1 using 2-acetylthiophene (General Procedure I) and n-propyl piperazine-1-carboxylate (General Procedure C1) as starting materials.

¹H NMR (400 MHz, DMSO-d₆) δ 8.49 (s, 1H), 8.23 (d, J=8.6 Hz, 1H), 8.16 (d, J=3.5 Hz, 1H), 8.02 (s, 1H), 7.81 (d, J=5.1 Hz, 1H), 7.70 (d, J=8.4 Hz, 1H), 7.30-7.18 (m, 1H), 3.98 (t, J=6.5 Hz, 2H), 3.78-3.34 (m, 8H), 1.59 (d, J=6.8 Hz, 2H), 0.89 (t, J=7.3 Hz, 3H).

LCMS (ESI-TOF) m/z 444.0 [M+H⁺] with a purity of >95%.

### Propyl (R)-4-(4-chloro-2-(thiophen-2-yl)quinoline-7-carbonyl)-2-methylpiperazine-1-carboxylate (B085)

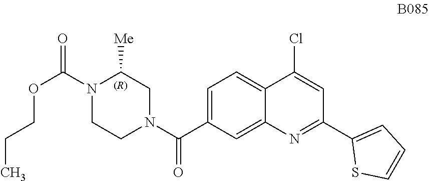

Intermediate from General Procedure K of the synthesis of B084 was reacted with (R)-n-propyl 2-methylpiperazine-1-carboxylic acid using General Procedure C1 to give compound B085.

¹H NMR (400 MHz, 80° C., DMSO-d₆) δ 8.35 (s, 1H), 8.23 (d, J=8.5 Hz, 1H), 8.07 (d, J=2.8 Hz, 1H), 7.98 (s, 1H), 7.76 (d, J=5.0 Hz, 1H), 7.67 (dd, J=8.5, 1.5 Hz, 1H), 7.23 (dd, J=5.0, 3.8 Hz, 1H), 4.36-3.67 (m, 6H), 3.39-3.08 (m, 3H), 1.67-1.51 (m, 2H), 1.13 (d, J=6.4 Hz, 3H), 0.89 (t, J=7.4 Hz, 3H).

LCMS (ESI-TOF) m/z 458.1 [M+H⁺] with a purity of >95%.

### Propyl (R)-4-(4-chloro-2-(1-methyl-1H-indazol-5-yl)quinoline-7-carbonyl)-2-methylpiperazine-1-carboxylate (B086)

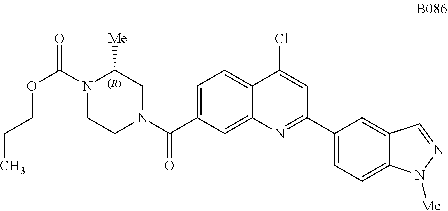

Compound B086 was synthesized according to General Procedure I, K and C1 using 1-(1-methyl-1H-indazol-5-yl)ethanone (General Procedure I) and (R)-n-propyl 2-methylpiperazine-1-carboxylate (General Procedure C1) as starting materials.

¹H NMR (400 MHz, 80° C., DMSO-d₆) δ 8.47 (s, 1H), 8.41 (dd, J=8.9, 1.6 Hz, 1H), 8.27 (d, J=8.6 Hz, 1H), 8.17 (s, 1H), 8.11 (d, J=1.0 Hz, 1H), 7.77 (d, J=8.9 Hz, 1H), 7.70 (dd, J=8.5, 1.5 Hz, 1H), 4.45-3.71 (m, 9H), 3.43-3.13 (m, 3H), 1.60 (dd, J=14.0, 6.7 Hz, 2H), 1.15 (d, J=6.7 Hz, 3H), 0.90 (t, J=7.4 Hz, 3H).

LCMS (ESI-TOF) m/z 506.2 [M+H⁺] with a purity of >97%.

### Propyl 4-(4-chloro-2-(4-fluoro-3-methoxyphenyl)quinoline-7-carbonyl)piperazine-1-carboxylate (B087)

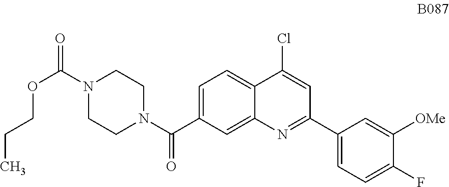 [SMILES: CCCOC(=O)N1CCN(C(=O)c2ccc3c(Cl)cc(-c4ccc(F)c(OC)c4)nc3c2)CC1]

Compound B087 was synthesized according to General Procedure I, K and C1 using 4-fluoro-3-methoxyacetophenone (General Procedure I) and n-propyl piperazine-1-carboxylate (General Procedure C1) as starting materials.

¹H NMR (400 MHz, 80° C., DMSO-d₆) δ 8.45 (s, 1H), 8.27 (d, J=8.4 Hz, 1H), 8.13 (d, J=1.0 Hz, 1H), 8.06 (dd, J=8.5, 2.0 Hz, 1H), 7.94-7.87 (m, 1H), 7.73 (dd, J=8.5, 1.5 Hz, 1H), 7.36 (dd, J=11.1, 8.6 Hz, 1H), 4.06-3.95 (m, 5H), 3.66-3.39 (m, 8H), 1.66-1.49 (m, 2H), 0.90 (t, J=7.4 Hz, 3H).

LCMS (ESI-TOF) m/z 486.1 [M+H⁺] with a purity of >99%.

### Propyl (R)-4-(4-chloro-2-(4-fluoro-3-methoxyphenyl)quinoline-7-carbonyl)-2-methylpiperazine-1-carboxylate (B088)

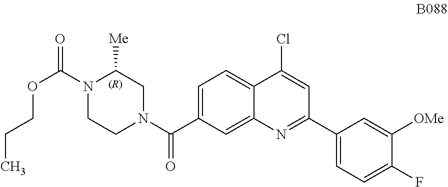

Compound B088 was synthesized according to C1 between intermediate K from synthesis of B087 and (R)-n-propyl 2-methylpiperazine-1-carboxylate (General Procedure C1) as starting materials.

¹H NMR (400 MHz, 80° C., DMSO-d₆) δ 8.44 (s, 1H), 8.28 (d, J=8.5 Hz, 1H), 8.11 (d, J=1.0 Hz, 1H), 8.06 (dd, J=8.4, 2.1 Hz, 1H), 7.91 (ddd, J=8.4, 4.4, 2.1 Hz, 1H), 7.72 (dd, J=8.5, 1.5 Hz, 1H), 7.35 (dd, J=11.2, 8.5 Hz, 1H), 4.36-3.75 (m, 9H), 3.39-3.11 (m, 3H), 1.66-1.54 (m, 2H), 1.14 (d, J=6.3 Hz, 3H), 0.89 (t, J=7.4 Hz, 3H).

LCMS (ESI-TOF) m/z 500.1 [M+H⁺] with a purity of >99%.

### Propyl (R)-4-(2-(4-(1H-imidazol-1-yl)phenyl)-4-chloroquinoline-7-carbonyl)-2-methylpiperazine-1-carboxylate (B089)

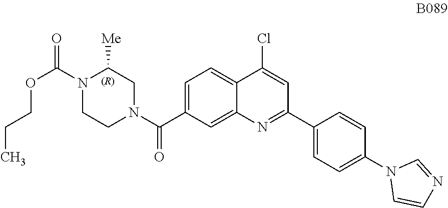

Compound B089 was synthesized according to General Procedure I, K and C1 using 4-(imidazol-1-yl)acetophenone (General Procedure I) and (R)-n-propyl 2-methylpiperazine-1-carboxylate (General Procedure C1) as starting materials.

¹H NMR (400 MHz, 80° C., DMSO-d₆) δ 8.46 (s, 1H), 8.45 (d, J=8.8 Hz, 2H), 8.31 (s, 1H), 8.29 (d, J=8.5 Hz, 1H), 8.13 (s, 1H), 7.83 (d, J=8.6 Hz, 2H), 7.79 (s, 1H), 7.73 (dd, J=8.5, 1.4 Hz, 1H), 7.14 (s, 1H), 4.38-3.61 (m, 6H), 3.40-3.10 (m, 3H), 1.66-1.51 (m, 2H), 1.15 (d, J=6.4 Hz, 3H), 0.90 (t, J=6.3 Hz, 3H).

LCMS (ESI-TOF) m/z 518.2 [M+H⁺] with a purity of >99%.

### Propyl (R)-4-(4-chloro-2-(4-morpholinophenyl)quinoline-7-carbonyl)-2-methylpiperazine-1-carboxylate (B090)

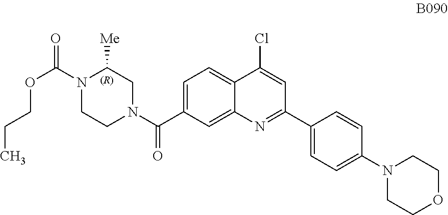

Compound B090 was synthesized according to General Procedure I, K and C1 using 4-morpholinoacetophenone (General Procedure I) and (R)-n-propyl 2-methylpiperazine-1-carboxylate (General Procedure C1) as starting materials.

¹H NMR (400 MHz, 80° C., DMSO-d₆) δ 8.27 (s, 1H), 8.20 (t, J=8.3 Hz, 3H), 8.02 (s, 1H), 7.63 (dd, J=8.5, 1.3 Hz, 1H), 7.07 (d, J=8.9 Hz, 2H), 4.37-3.72 (m, 10H), 3.36-3.23 (m, 5H), 3.16 (dd, J=27.3, 13.6 Hz, 2H), 1.66-1.52 (m, 2H), 1.14 (d, J=6.5 Hz, 3H), 0.89 (t, J=7.4 Hz, 3H).

LCMS (ESI-TOF) m/z 537.2 [M+H⁺] with a purity of >99%.

### Propyl (S)-4-(4-chloro-2-(1-methyl-1H-pyrazol-5-yl)quinoline-7-carbonyl)-3-methylpiperazine-1-carboxylate (B091)

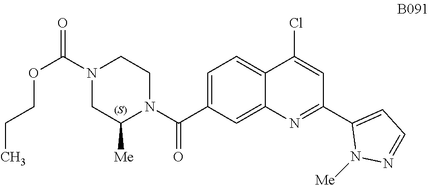

Compound B091 was synthesized according to General Procedure L using propyl (S)-4-(2,4-dichloroquinoline-7-carbonyl)-3-methylpiperazine-1-carboxylate and 1-methylpyrazole-5-boronic acid as starting materials.

¹H NMR (400 MHz, 80° C., DMSO-d₆) δ 8.27 (d, J=8.5 Hz, 1H), 8.21 (s, 1H), 8.11 (d, J=1.1 Hz, 1H), 7.73 (dd, J=8.5, 1.6 Hz, 1H), 7.54 (d, J=2.0 Hz, 1H), 7.11 (d, J=2.0 Hz, 1H), 4.40-4.36 (m, 1H), 4.33 (s, 3H), 4.09-3.74 (m, 5H), 3.30-2.93 (m, 3H), 1.67-1.53 (m, 2H), 1.21 (d, J=6.8 Hz, 3H), 0.89 (t, J=7.4 Hz, 3H). LCMS (ESI-TOF) m/z 456.1 [M+H⁺] with a purity of >99%.

### Propyl 4-(2-(4-carbamoylphenyl)-4-chloro-3-methylquinoline-7-carbonyl)piperazine-1-carboxylate (B092)

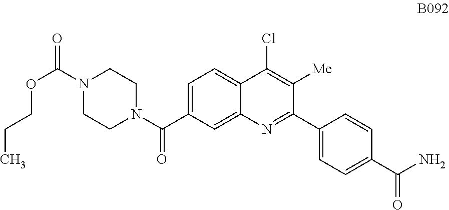 [SMILES: CCCOC(=O)N1CCN(C(=O)c2ccc3c(Cl)c(C)c(-c4ccc(C(N)=O)cc4)nc3c2)CC1]

Compound B092 was synthesized according to General Procedure I, K and C1 using 4-propanoylbenzonitrile (General Procedure I) and n-propyl piperazine-1-carboxylate (General Procedure C1) as starting materials. Compound B092 is a side product of the synthesis of propyl 4-(4-chloro-2-(4-cyanophenyl)-3-methylquinoline-7-carbonyl)piperazine-1-carboxylate.

¹H NMR (400 MHz, 80° C., DMSO-d₆) δ 8.29 (d, J=8.6 Hz, 1H), 8.06 (s, 1H), 8.01 (d, J=8.3 Hz, 2H), 7.76-7.72 (m, 1H), 7.68 (d, J=8.3 Hz, 2H), 3.99 (t, J=6.6 Hz, 2H), 3.62-3.35 (m, 8H), 2.51 (s, 3H), 1.78-1.47 (m, 2H), 0.89 (t, J=7.4 Hz, 3H).

LCMS (ESI-TOF) m/z 495.1 [M+H⁺] with a purity of >97%.

### Propyl 4-(2-(4-(aminomethyl)phenyl)-4-chloro-3-methylquinoline-7-carbonyl)piperazine-1-carboxylate (B093)

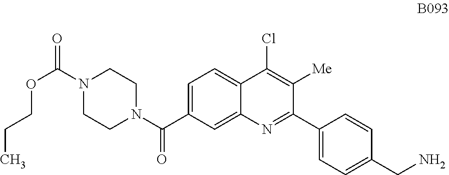 [SMILES: CCCOC(=O)N1CCN(C(=O)c2ccc3c(Cl)c(C)c(-c4ccc(CN)cc4)nc3c2)CC1]

Compound B093 was synthesized according to General Procedure D using propyl 4-(4-chloro-2-(4-cyanophenyl)-3-methylquinoline-7-carbonyl)piperazine-1-carboxylate as starting material (synthesized for B092)

¹H NMR (400 MHz, 80° C., DMSO-d₆) δ 8.26 (d, J=8.6 Hz, 1H), 8.03 (s, 1H), 7.72 (d, J=8.6 Hz, 1H), 7.54 (d, J=8.1 Hz, 2H), 7.48 (d, J=8.0 Hz, 2H), 3.99 (t, J=6.5 Hz, 2H), 3.84 (s, 2H), 3.67-3.26 (m, 8H), 2.52 (s, 3H), 1.74 (br s, 2H), 1.65-1.37 (m, 2H), 0.89 (t, J=7.4 Hz, 3H).

LCMS (ESI-TOF) m/z 481.2 [M+H⁺] with a purity of >99%.

### Propyl (R)-4-(4-chloro-2-(4-(N,N-dimethylsulfamoyl)phenyl)quinoline-7-carbonyl)-2-methylpiperazine-1-carboxylate (B094)

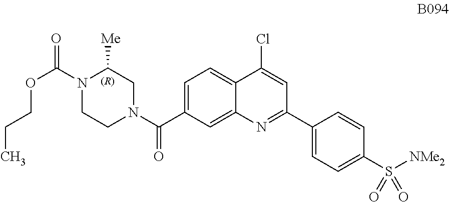

Compound B094 was synthesized according to General Procedure I, K and C1 using 4-acetyl-N,N-dimethyl-benzenesulfonamide (General Procedure I) and (R)-n-propyl 2-methylpiperazine-1-carboxylate (General Procedure C1) as starting materials.

¹H NMR (400 MHz, 80° C., DMSO-d₆) δ 8.53 (d, J=8.1 Hz, 2H), 8.49 (s, 1H), 8.32 (d, J=8.3 Hz, 1H), 8.16 (s, 1H), 7.92 (d, J=7.9 Hz, 2H), 7.77 (d, J=8.4 Hz, 1H), 4.36-3.69 (m, 6H), 3.41-3.10 (m, 3H), 2.71 (s, 6H), 1.66-1.47 (m, 2H), 1.15 (s, 3H), 0.89 (t, J=7.3 Hz, 3H).

LCMS (ESI-TOF) m/z 559.1 [M+H⁺] with a purity of >99%.

### Propyl (R)-4-(4-chloro-2-(4-(4-methylpiperazin-1-yl)phenyl)quinoline-7-carbonyl)-2-methylpiperazine-1-carboxylate (B095)

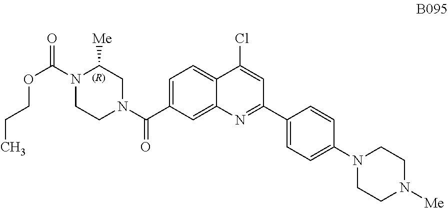

Compound B095 was synthesized according to General Procedure I, K and C1 using 4-(4-methylpiperazino)acetophenone (General Procedure I) and (R)-n-propyl 2-methylpiperazine-1-carboxylate (General Procedure C1) as starting materials.

¹H NMR (400 MHz, 80° C., DMSO-d₆) δ 8.27 (s, 1H), 8.21 (d, J=8.5 Hz, 1H), 8.17 (d, J=8.9 Hz, 2H), 8.01 (s, 1H), 7.63 (d, J=7.5 Hz, 1H), 7.05 (d, J=8.8 Hz, 2H), 4.37-3.63 (m, 6H), 3.21-3.09 (m, 2H), 3.05 (s, 9H), 2.24 (s, 3H), 1.67-1.52 (m, 2H), 1.13 (d, J=5.9 Hz, 3H), 0.89 (t, J=7.4 Hz, 3H).

LCMS (ESI-TOF) m/z 550.2 [M+H⁺] with a purity of >99%.

### Propyl (R)-4-(2-(3-(4H-1,2,4-triazol-4-yl)phenyl)-4-chloroquinoline-7-carbonyl)-2-methylpiperazine-1-carboxylate (B096)

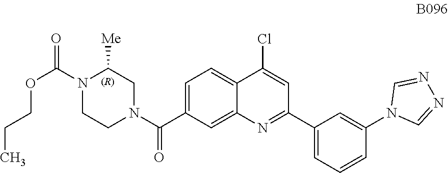

Compound B096 was synthesized according to General Procedure I, K and C1 using 1-[3-(4H-1,2,4-triazol-4-yl)phenyl]ethan-1-one (General Procedure I) and (R)-n-propyl 2-methylpiperazine-1-carboxylate (General Procedure C1) as starting materials.

¹H NMR (400 MHz, 80° C., DMSO-d₆) δ 9.16 (s, 2H), 8.59 (s, 1H), 8.53 (s, 1H), 8.41 (d, J=7.9 Hz, 1H), 8.31 (d, J=8.6 Hz, 1H), 8.16 (s, 1H), 7.84 (d, J=9.1 Hz, 1H), 7.79-7.69 (m, 2H), 4.40-3.65 (m, 6H), 3.37-3.08 (m, 3H), 1.67-1.53 (m, 2H), 1.14 (d, J=6.2 Hz, 3H), 0.89 (t, J=7.4 Hz, 3H).

LCMS (ESI-TOF) m/z 519.1 [M+H⁺] with a purity of >99%.

### Propyl 4-(2-(3-(aminomethyl)-4-methoxyphenyl)-4-chloroquinoline-7-carbonyl)piperazine-1-carboxylate (B097)

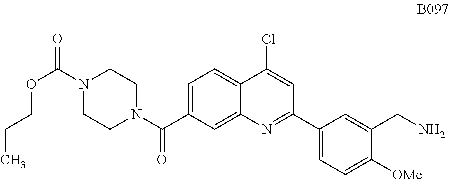 [SMILES: CCCOC(=O)N1CCN(C(=O)c2ccc3c(Cl)cc(-c4ccc(OC)c(CN)c4)nc3c2)CC1]

Compound B097 was synthesized according to General Procedure L and then General Procedure D using propyl 4-(2,4-dichloroquinoline-7-carbonyl)piperazine-1-carboxylate and 3-cyano-4-methoxyphenyl boronic acid as starting materials (General Procedure L).

¹H NMR (400 MHz, DMSO-d₆) δ 8.44 (s, 1H), 8.33 (s, 1H), 8.23 (dd, J=13.1, 5.3 Hz, 2H), 8.10 (s, 1H), 7.75-7.65 (m, 1H), 7.13 (d, J=8.7 Hz, 1H), 3.98 (t, J=6.5 Hz, 2H), 3.89 (s, 3H), 3.78 (s, 2H), 3.73-3.37 (m, 10H), 1.59 (dd, J=13.7, 7.1 Hz, 2H), 0.89 (t, J=7.1 Hz, 3H).

LCMS (ESI-TOF) m/z 497.2 [M+H⁺] with a purity of >98%.

### Propyl (R)-4-(4-chloro-2-(4-((dimethylamino)methyl)phenyl)quinoline-7-carbonyl)-2-methylpiperazine-1-carboxylate (B098)

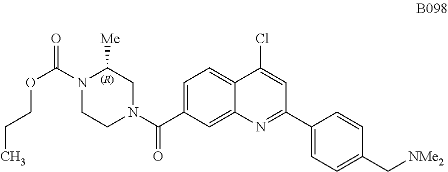

Compound B098 was synthesized according to General Procedure I, K and C1 using 1-[4-(dimethylaminomethyl)phenyl]ethan-1-one (General Procedure I) and (R)-n-propyl 2-methylpiperazine-1-carboxylate (General Procedure C1) as starting materials.

¹H NMR (400 MHz, 80° C., DMSO-d₆) δ 8.37 (s, 1H), 8.27 (d, J=8.6 Hz, 1H), 8.24 (d, J=8.2 Hz, 2H), 8.10 (s, 1H), 7.71 (d, J=8.4 Hz, 1H), 7.48 (d, J=8.2 Hz, 2H), 4.40-3.62 (m, 6H), 3.49 (s, 2H), 3.34-3.13 (m, 3H), 2.21 (s, 6H), 1.72-1.51 (m, 2H), 1.14 (d, J=6.6 Hz, 3H), 0.89 (t, J=7.4 Hz, 3H).

LCMS (ESI-TOF) m/z 509.2 [M+H⁺] with a purity of >99%.

### Propyl 4-(4-chloro-2-(4-methoxy-3-methylphenyl)quinoline-7-carbonyl)piperazine-1-carboxylate (B099)

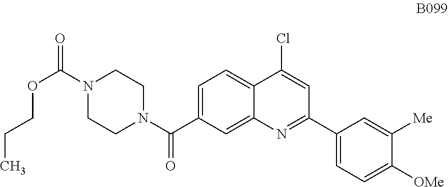 [SMILES: CCCOC(=O)N1CCN(C(=O)c2ccc3c(Cl)cc(-c4ccc(OC)c(C)c4)nc3c2)CC1]

Compound B099 was synthesized according to General Procedure L using propyl 4-(2,4-dichloroquinoline-7-carbonyl)piperazine-1-carboxylate and 4-methoxy-3-methylphenyl boronic acid as starting materials.

¹H NMR (400 MHz, DMSO-d₆) δ 8.42 (s, 1H), 8.24 (d, J=8.5 Hz, 1H), 8.21-8.11 (m, 2H), 8.09 (s, 1H), 7.72-7.66 (m, 1H), 7.12 (d, J=9.3 Hz, 1H), 3.98 (t, J=6.6 Hz, 2H), 3.89 (s, 3H), 3.73-3.37 (m, 8H), 2.27 (s, 3H), 1.59 (dd, J=13.8, 6.7 Hz, 2H), 0.89 (t, J=7.3 Hz, 3H).

LCMS (ESI-TOF) m/z 482.1 [M+H⁺] with a purity of >99%.

### Propyl 4-(4-chloro-2-(3-(hydroxymethyl)-4-methoxyphenyl)quinoline-7-carbonyl)piperazine-1-carboxylate (B100)

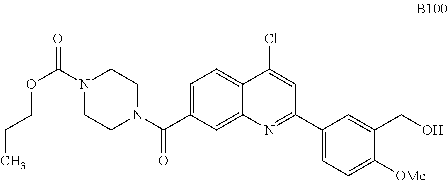 [SMILES: CCCOC(=O)N1CCN(C(=O)c2ccc3c(Cl)cc(-c4ccc(OC)c(CO)c4)nc3c2)CC1]

Compound B100 was synthesized according to General Procedure L using propyl 4-(2,4-dichloroquinoline-7-carbonyl)piperazine-1-carboxylate and 3-hydroxymethyl-4-methoxyphenyl boronic acid as starting materials.

¹H NMR (400 MHz, DMSO-d₆) δ 8.39 (s, 2H), 8.25 (d, J=8.5 Hz, 1H), 8.21 (dd, J=8.7, 2.2 Hz, 1H), 8.11 (s, 1H), 7.70 (dd, J=8.5, 1.4 Hz, 1H), 7.13 (d, J=8.7 Hz, 1H), 5.16 (t, J=5.5 Hz, 1H), 4.59 (d, J=5.1 Hz, 2H), 3.98 (t, J=6.6 Hz, 2H), 3.88 (s, 3H), 3.81-3.34 (m, 8H), 1.59 (dd, J=13.9, 6.9 Hz, 2H), 0.89 (t, J=7.2 Hz, 3H).

LCMS (ESI-TOF) m/z 496.1 [M+H⁺] with a purity of >97%.

### Propyl 4-(4-chloro-2-(4-cyano-3-methylphenyl)quinoline-7-carbonyl)piperazine-1-carboxylate (B101)

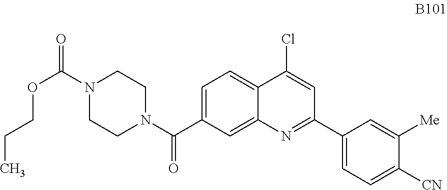 [SMILES: CCCOC(=O)N1CCN(C(=O)c2ccc3c(Cl)cc(-c4ccc(C#N)c(C)c4)nc3c2)CC1]

Compound B101 was synthesized according to General Procedure L using propyl 4-(2,4-dichloroquinoline-7-carbonyl)piperazine-1-carboxylate and 4-cyano-3-methylphenyl boronic acid as starting materials.

¹H NMR (400 MHz, DMSO-d₆) δ 8.59 (s, 1H), 8.44 (s, 1H), 8.32 (t, J=7.7 Hz, 2H), 8.19 (s, 1H), 7.98 (d, J=8.2 Hz, 1H), 7.85-7.77 (m, 1H), 3.98 (t, J=6.6 Hz, 2H), 3.73-3.36 (m, 8H), 2.62 (s, 3H), 1.59 (dd, J=13.8, 7.0 Hz, 2H), 0.89 (t, J=7.3 Hz, 3H).

LCMS (ESI-TOF) m/z 477.1 [M+H⁺] with a purity of >99%.

### Propyl 4-(4-chloro-2-(3-(dimethylcarbamoyl)phenyl)quinoline-7-carbonyl)piperazine-1-carboxylate (B102)

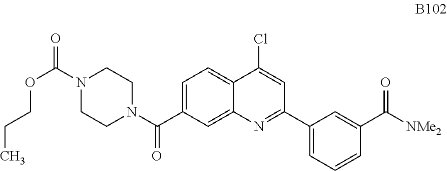 [SMILES: CCCOC(=O)N1CCN(C(=O)c2ccc3c(Cl)cc(-c4cccc(C(=O)N(C)C)c4)nc3c2)CC1]

Compound B102 was synthesized according to General Procedure L using propyl 4-(2,4-dichloroquinoline-7-carbonyl)piperazine-1-carboxylate and 3-(dimethylcarbamoyl)phenyl boronic acid as starting materials.

¹H NMR (400 MHz, DMSO-d₆) δ 8.56 (s, 1H), 8.40 (d, J=7.9 Hz, 1H), 8.36 (s, 1H), 8.30 (d, J=8.5 Hz, 1H), 8.18 (s, 1H), 7.77 (dd, J=8.6, 1.5 Hz, 1H), 7.64 (t, J=7.7 Hz, 1H), 7.57 (d, J=7.6 Hz, 1H), 3.98 (t, J=6.6 Hz, 2H), 3.79-3.35 (m, 8H), 3.11-2.91 (m, 6H), 1.59 (dd, J=13.6, 6.8 Hz, 2H), 0.89 (t, J=7.2 Hz, 3H).

LCMS (ESI-TOF) m/z 509.2 [M+H⁺] with a purity of >99%.

### Propyl 4-(4-chloro-2-(4-sulfamoylphenyl)quinoline-7-carbonyl)piperazine-1-carboxylate (B103)

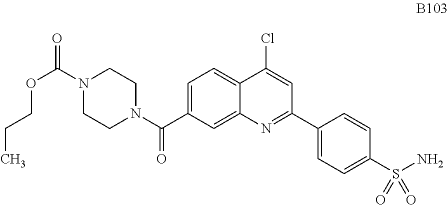 [SMILES: CCCOC(=O)N1CCN(C(=O)c2ccc3c(Cl)cc(-c4ccc(S(N)(=O)=O)cc4)nc3c2)CC1]

Compound B103 was synthesized according to General Procedure L using propyl 4-(2,4-dichloroquinoline-7-carbonyl)piperazine-1-carboxylate and 4-sulfamoylphenyl boronic acid as starting materials.

¹H NMR (400 MHz, DMSO-d₆) δ 8.58 (s, 1H), 8.52 (d, J=8.5 Hz, 2H), 8.32 (d, J=8.6 Hz, 1H), 8.19 (s, 1H), 8.01 (d, J=8.4 Hz, 2H), 7.80 (d, J=8.5 Hz, 1H), 7.48 (s, 2H), 3.98 (t, J=6.6 Hz, 2H), 3.85-3.34 (m, 8H), 1.59 (dd, J=13.9, 7.1 Hz, 2H), 0.89 (t, J=7.3 Hz, 3H).

LCMS (ESI-TOF) m/z 517.1 [M+H⁺] with a purity of >99%.

### Propyl 4-(4-chloro-2-(3-((dimethylamino)methyl)-4-methoxyphenyl)quinoline-7-carbonyl)piperazine-1-carboxylate (B104)

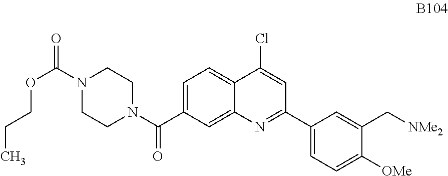 [SMILES: CCCOC(=O)N1CCN(C(=O)c2ccc3c(Cl)cc(-c4ccc(OC)c(CN(C)C)c4)nc3c2)CC1]

Compound B104 was synthesized according to General Procedure L using propyl 4-(2,4-dichloroquinoline-7-carbonyl)piperazine-1-carboxylate and [3-(dimethylaminomethyl)-4-methoxyphenyl]boronic acid as starting materials.

¹H NMR (400 MHz, DMSO-d₆) δ 8.40 (s, 1H), 8.34-8.18 (m, 3H), 8.11 (s, 1H), 7.70 (dd, J=8.5, 1.4 Hz, 1H), 7.16 (d, J=8.7 Hz, 1H), 3.98 (t, J=6.6 Hz, 2H), 3.88 (s, 3H), 3.72-3.37 (m, 10H), 2.21 (s, 6H), 1.59 (dd, J=13.9, 7.0 Hz, 2H), 0.89 (t, J=7.2 Hz, 3H).

LCMS (ESI-TOF) m/z 525.2 [M+H⁺] with a purity of >98%.

### Propyl 4-(4-chloro-2-(3-(morpholinomethyl)phenyl)quinoline-7-carbonyl)piperazine-1-carboxylate (B105)

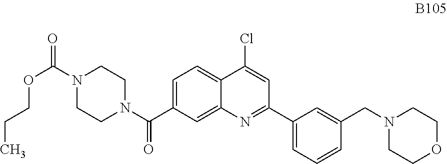 [SMILES: CCCOC(=O)N1CCN(C(=O)c2ccc3c(Cl)cc(-c4cccc(CN5CCOCC5)c4)nc3c2)CC1]

Compound B105 was synthesized according to General Procedure L using propyl 4-(2,4-dichloroquinoline-7-carbonyl)piperazine-1-carboxylate and 3-(morpholin-4-ylmethyl)phenyl boronic acid as starting materials.

¹H NMR (400 MHz, DMSO-d₆) δ 8.47 (s, 1H), 8.29 (d, J=8.6 Hz, 1H), 8.26 (s, 1H), 8.21 (d, J=7.4 Hz, 1H), 8.16 (s, 1H), 7.75 (d, J=8.5 Hz, 1H), 7.58-7.47 (m, 2H), 3.98 (t, J=6.6 Hz, 2H), 3.82-3.35 (m, 14H), 2.41 (s, 4H), 1.59 (dd, J=14.2, 7.1 Hz, 2H), 0.89 (t, J=7.3 Hz, 3H).

LCMS (ESI-TOF) m/z 537.2 [M+H⁺] with a purity of >99%.

### Propyl (R)-4-(4-chloro-2-(3-cyano-4-methoxyphenyl)quinoline-7-carbonyl)-2-methylpiperazine-1-carboxylate (B106)

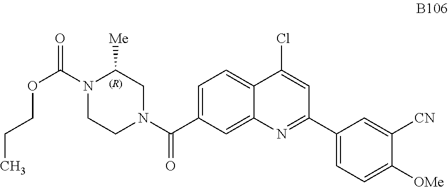

Compound B106 was synthesized according to General Procedure L using propyl (R)-4-(2,4-dichloroquinoline-7-carbonyl)-2-methylpiperazine-1-carboxylate and 3-cyano-4-methoxyphenyl boronic acid as starting materials.

¹H NMR (400 MHz, 80° C., DMSO-d₆) δ 8.69-8.58 (m, 2H), 8.48 (s, 1H), 8.27 (d, J=8.6 Hz, 1H), 8.11 (s, 1H), 7.72 (dd, J=8.5, 1.5 Hz, 1H), 7.43 (d, J=8.9 Hz, 1H), 4.34-3.69 (m, 9H), 3.35-3.08 (m, 3H), 1.70-1.49 (m, 2H), 1.14 (d, J=5.7 Hz, 3H), 0.89 (t, J=7.4 Hz, 3H).

LCMS (ESI-TOF) m/z 507.1 [M+H⁺] with a purity of >99%.

### Propyl (R)-4-(2-(3-(aminomethyl)-4-methoxyphenyl)-4-chloroquinoline-7-carbonyl)-2-methylpiperazine-1-carboxylate (B107)

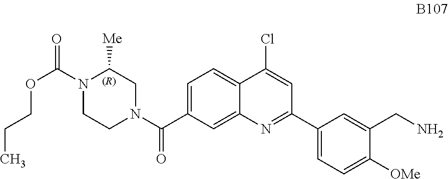

Compound B107 was synthesized according to General Procedure D using compound B106 as starting material.

¹H NMR (400 MHz, 80° C., DMSO-d₆) δ 8.34 (s, 1H), 8.28 (d, J=2.2 Hz, 1H), 8.24 (d, J=8.5 Hz, 1H), 8.18 (dd, J=8.5, 2.2 Hz, 1H), 8.07 (s, 1H), 7.67 (d, J=8.5 Hz, 1H), 7.12 (d, J=8.6 Hz, 1H), 4.35-3.62 (m, 11H), 3.36-3.12 (m, 3H), 1.70-1.53 (m, 4H), 1.13 (s, 3H), 0.89 (t, J=7.4 Hz, 3H).

LCMS (ESI-TOF) m/z 511.1 [M+H⁺] with a purity of >98%.

### Propyl 4-(2-(4-(aminomethyl)-3-methylphenyl)-4-chloroquinoline-7-carbonyl)piperazine-1-carboxylate (B108)

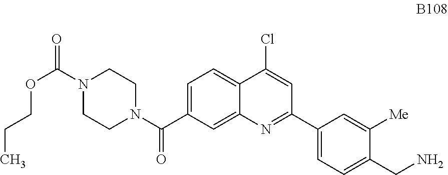 [SMILES: CCCOC(=O)N1CCN(C(=O)c2ccc3c(Cl)cc(-c4ccc(CN)c(C)c4)nc3c2)CC1]

Compound B108 was synthesized according to General Procedure D using compound B101 as starting material.

¹H NMR (400 MHz, 80° C., DMSO-d₆) δ 8.37-8.31 (m, 1H), 8.29-8.22 (m, 1H), 8.11 (d, J=1.1 Hz, 1H), 8.09-7.98 (m, 2H), 7.74-7.65 (m, 1H), 7.58-7.44 (m, 1H), 4.06 (dt, J=26.9, 6.4 Hz, 2H), 3.81 (s, 2H), 3.69-3.32 (m, 8H), 2.43-2.34 (m, 3H), 1.71-1.53 (m, 4H), 0.99-0.77 (m, 3H).

LCMS (ESI-TOF) m/z 481.2 [M+H⁺] with a purity of >98%.

### Propyl (R)-4-(4-chloro-2-(1-methylcyclopropyl)quinoline-7-carbonyl)-2-methylpiperazine-1-carboxylate (B109)

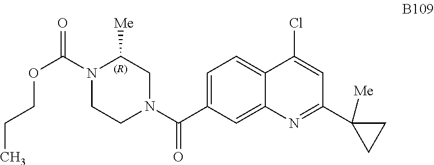

Step 1: Intermediate ethyl 4-chloro-2-(prop-1-en-2-yl)quinoline-7-carboxylate was synthesized under the same conditions in General Procedure L using ethyl 2,4-dichloroquinoline-7-carboxylate and prop-1-en-2-ylboronic acid as starting material.

Step 2: To a stirred suspension of trimethylsulfoxonium iodide (1.2 g, 5.44 mmol) in dimethylsulfoxide (10 mL) and tetrahydrofuran (10 mL) was added potassium tert-butoxide (610 g, 5.44 mmol) in one portion at room temperature. After 30 min at the same temperature, a solution of ethyl 4-chloro-2-(prop-1-en-2-yl)quinoline-7-carboxylate (1 g, 3.63 mmol) in tetrahydrofuran (10 mL) was added. The resulting mixture was stirred at room temperature for 18 h and then quenched with water (20 mL) and extracted with ethyl acetate (3×100 mL). The combined organic layer was washed with brine and dried over anhydrous sodium sulfate, filtered, then concentrated to afford crude ethyl 4-chloro-2-(1-methylcyclopropyl)quinoline-7-carboxylate (1 g, 95%) as a brown color gum.

Step 3: 4-Chloro-2-(1-methylcyclopropyl)quinoline-7-carboxylic acid was synthesized from crude ethyl 4-chloro-2-(1-methylcyclopropyl)quinoline-7-carboxylate using General Procedure K.

Step 4: Compound B109 was synthesized according to General Procedure C1 using 4-chloro-2-(1-methylcyclopropyl)quinoline-7-carboxylic acid and (R)-n-propyl 2-methylpiperazine-1-carboxylate as starting materials.

¹H NMR (400 MHz, 80° C., DMSO-d₆) δ 8.20 (d, J=8.0 Hz, 1H), 7.99 (s, 1H), 7.55 (dd, J=1.6, 8.8 Hz, 1H), 7.54 (s, 1H), 4.75-2.95 (m, 9H), 1.66 (q, J=7.2 Hz, 2H), 1.61 (s, 3H), 1.45-1.35 (m, 2H), 1.35-1.05 (m, 3H), 1.00-0.90 (m, 5H).

LCMS (ESI-TOF) m/z 430.2 [M+H⁺] with a purity of >98%.

### Propyl 4-(4-chloro-2-(4-((dimethylamino)methyl)phenyl)quinoline-7-carbonyl)piperazine-1-carboxylate (B110)

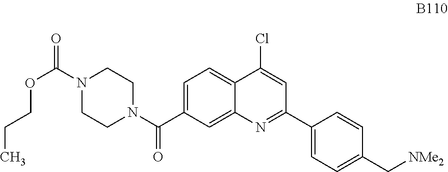 [SMILES: CCCOC(=O)N1CCN(C(=O)c2ccc3c(Cl)cc(-c4ccc(CN(C)C)cc4)nc3c2)CC1]

Compound B110 was synthesized according to General Procedure I, K and C1 using 1-[4-(dimethylaminomethyl)phenyl]ethan-1-one (General Procedure I) and n-propyl piperazine-1-carboxylate (General Procedure C1) as starting materials.

¹H NMR (400 MHz, 80° C., DMSO-d₆) δ 8.36 (s, 1H), 8.25 (dd, J=10.8, 8.5 Hz, 3H), 8.12 (s, 1H), 7.72 (d, J=8.6 Hz, 1H), 7.48 (d, J=8.1 Hz, 2H), 4.00 (t, J=6.6 Hz, 2H), 3.64-3.34 (m, 10H), 2.21 (s, 6H), 1.65-1.53 (m, 2H), 0.90 (t, J=7.4 Hz, 3H).

LCMS (ESI-TOF) m/z 495.2 [M+H⁺] with a purity of >97%.

### Propyl (R)-4-(4-chloro-2-(3-((dimethylamino)methyl)phenyl)quinoline-7-carbonyl)-2-methylpiperazine-1-carboxylate (B111)

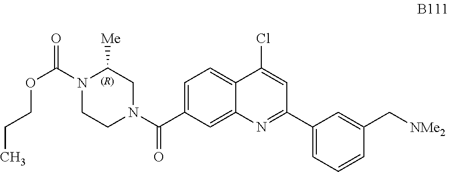

Compound B111 was synthesized according to General Procedure I, K and C1 using 1-[3-(dimethylaminomethyl)phenyl]ethan-1-one (General Procedure I) and (R)-n-propyl 2-methylpiperazine-1-carboxylate (General Procedure C1) as starting materials.

¹H NMR (400 MHz, 80° C., DMSO-d₆) δ 8.36 (s, 1H), 8.28 (d, J=8.5 Hz, 1H), 8.20 (s, 1H), 8.15 (d, J=7.7 Hz, 1H), 8.12 (s, 1H), 7.72 (d, J=7.1 Hz, 1H), 7.55-7.43 (m, 2H), 4.40-3.65 (m, 6H), 3.52 (s, 2H), 3.40-3.17 (m, 3H), 2.22 (s, 6H), 1.69-1.52 (m, 2H), 1.14 (d, J=6.4 Hz, 3H), 0.89 (t, J=7.4 Hz, 3H).

LCMS (ESI-TOF) m/z 509.2 [M+H⁺] with a purity of >98%.

### Propyl 4-(4-chloro-2-(3-((dimethylamino)methyl)phenyl)quinoline-7-carbonyl)piperazine-1-carboxylate (B112)

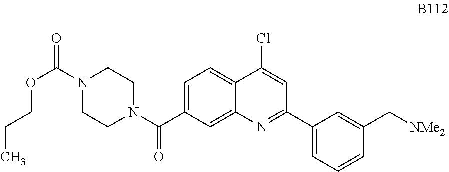 [SMILES: CCCOC(=O)N1CCN(C(=O)c2ccc3c(Cl)cc(-c4cccc(CN(C)C)c4)nc3c2)CC1]

Compound B112 was synthesized according to General Procedure I, K and C1 using 1-[3-(dimethylaminomethyl)phenyl]ethan-1-one (General Procedure I) and n-propyl piperazine-1-carboxylate (General Procedure C1) as starting materials.

¹H NMR (400 MHz, 80° C., DMSO-d₆) δ 8.36 (s, 1H), 8.27 (d, J=8.5 Hz, 1H), 8.20 (s, 1H), 8.17-8.12 (m, 2H), 7.72 (d, J=8.5 Hz, 1H), 7.55-7.41 (m, 2H), 3.99 (t, J=6.6 Hz, 2H), 3.64-3.41 (m, 10H), 2.22 (s, 6H), 1.67-1.52 (m, 2H), 0.90 (t, J=7.4 Hz, 3H).

LCMS (ESI-TOF) m/z 495.2 [M+H⁺] with a purity of >96%.

### Propyl (R)-4-(2-(4-carbamoylphenyl)-4-chloro-3-methylquinoline-7-carbonyl)-2-methylpiperazine-1-carboxylate (B113)

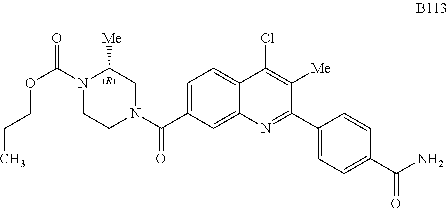

Compound B113 was synthesized according to General Procedure I, K and C1 using 4-propanoylbenzonitrile (General Procedure I) and (R)-n-propyl 2-methylpiperazine-1-carboxylate (General Procedure C1) as starting materials. Compound B113 is a side product of the synthesis of compound B114.

¹H NMR (400 MHz, 80° C., DMSO-d₆) δ 8.29 (d, J=8.6 Hz, 1H), 8.04 (s, 1H), 8.02 (d, J=8.3 Hz, 2H), 7.74 (dd, J=8.6, 1.5 Hz, 1H), 7.68 (d, J=8.3 Hz, 2H), 4.37-3.61 (m, 6H), 3.40-3.13 (m, 3H), 2.51 (s, 3H), 1.66-1.50 (m, 2H), 1.09 (t, J=16.6 Hz, 3H), 0.89 (t, J=7.4 Hz, 3H).

LCMS (ESI-TOF) m/z 509.2 [M+H⁺] with a purity of >98%.

### Propyl (R)-4-(4-chloro-2-(4-cyanophenyl)-3-methylquinoline-7-carbonyl)-2-methylpiperazine-1-carboxylate (B114)

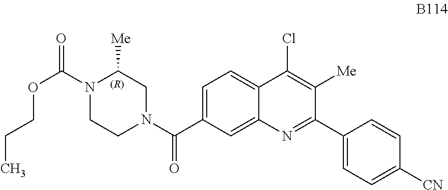

Compound B114 was synthesized according to General Procedure I, K and C1 using 4-propanoylbenzonitrile (General Procedure I) and (R)-n-propyl 2-methylpiperazine-1-carboxylate (General Procedure C1) as starting materials.

¹H NMR (400 MHz, 80° C., DMSO-d₆) δ 8.30 (d, J=8.6 Hz, 1H), 8.05 (s, 1H), 7.97 (d, J=8.3 Hz, 2H), 7.82 (d, J=8.3 Hz, 2H), 7.76 (d, J=8.6 Hz, 1H), 4.28-3.73 (m, 6H), 3.34-3.17 (d, J=9.4 Hz, 3H), 2.50 (s, 3H), 1.59 (dd, J=14.1, 6.8 Hz, 2H), 1.12 (d, J=6.4 Hz, 3H), 0.89 (t, J=7.4 Hz, 3H).

LCMS (ESI-TOF) m/z 491.2 [M+H⁺] with a purity of >98%.

### Propyl 4-(4-chloro-2-(2-methylpyridin-4-yl)quinoline-7-carbonyl)piperazine-1-carboxylate (B115)

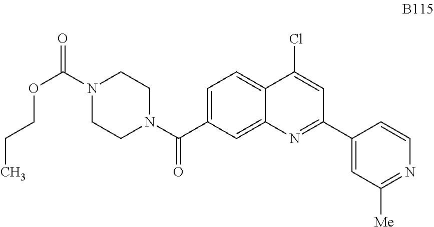 [SMILES: CCCOC(=O)N1CCN(C(=O)c2ccc3c(Cl)cc(-c4ccnc(C)c4)nc3c2)CC1]

Compound B115 was synthesized according to General Procedure L using propyl 4-(2,4-dichloroquinoline-7-carbonyl)piperazine-1-carboxylate and 2-methylpyridin-4-ylboronic acid as starting materials.

¹H NMR (400 MHz, DMSO-d₆) δ 8.65 (d, J=5.2 Hz, 1H), 8.58 (s, 1H), 8.32 (d, J=8.6 Hz, 1H), 8.21 (s, 1H), 8.16 (s, 1H), 8.07 (d, J=5.1 Hz, 1H), 7.81 (dd, J=8.6, 1.3 Hz, 1H), 3.98 (t, J=6.6 Hz, 2H), 3.76-3.36 (m, 8H), 2.60 (d, J=8.1 Hz, 3H), 1.59 (dd, J=14.0, 7.1 Hz, 2H), 0.89 (t, J=7.3 Hz, 3H).

LCMS (ESI-TOF) m/z 453.1 [M+H⁺] with a purity of >99%.

### Propyl 4-(4-chloro-2-(4-(N-methylsulfamoyl)phenyl)quinoline-7-carbonyl)piperazine-1-carboxylate (B116)

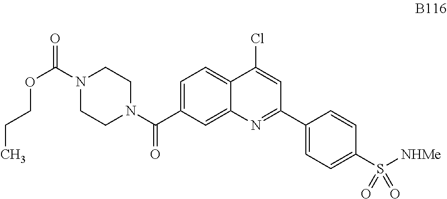 [SMILES: CCCOC(=O)N1CCN(C(=O)c2ccc3c(Cl)cc(-c4ccc(S(=O)(=O)NC)cc4)nc3c2)CC1]

Compound B116 was synthesized according to General Procedure L using propyl 4-(2,4-dichloroquinoline-7-carbonyl)piperazine-1-carboxylate and 4-(methylsulfamoyl)phenyl boronic acid as starting materials.

¹H NMR (400 MHz, DMSO-d₆) δ 8.58 (s, 1H), 8.54 (d, J=7.8 Hz, 1H), 8.32 (d, J=8.5 Hz, 1H), 8.19 (s, 1H), 7.96 (d, J=8.4 Hz, 2H), 7.84-7.76 (m, 1H), 7.59 (s, 1H), 3.98 (t, J=6.6 Hz, 2H), 3.78-3.36 (m, 8H), 2.48 (s, 3H), 1.59 (dd, J=13.8, 6.8 Hz, 2H), 0.94-0.78 (m, 3H).

LCMS (ESI-TOF) m/z 531.1 [M+H⁺] with a purity of >99%.

### Propyl 4-(4-chloro-2-(3-sulfamoylphenyl)quinoline-7-carbonyl)piperazine-1-carboxylate (B117)

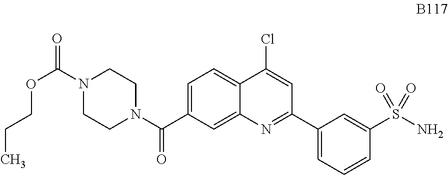 [SMILES: CCCOC(=O)N1CCN(C(=O)c2ccc3c(Cl)cc(-c4cccc(S(N)(=O)=O)c4)nc3c2)CC1]

Compound B117 was synthesized according to General Procedure L using propyl 4-(2,4-dichloroquinoline-7-carbonyl)piperazine-1-carboxylate and (3-sulfamoylphenyl)boronic acid as starting materials.

¹H NMR (400 MHz, DMSO-d₆) δ 8.80 (s, 1H), 8.56-8.47 (m, 2H), 8.32 (d, J=8.6 Hz, 1H), 8.20 (s, 1H), 7.99 (d, J=7.6 Hz, 1H), 7.83-7.74 (m, 2H), 7.48 (s, 2H), 3.98 (t, J=6.6 Hz, 2H), 3.81-3.35 (m, 8H), 1.59 (dd, J=14.1, 7.0 Hz, 2H), 0.89 (t, J=7.3 Hz, 3H).

LCMS (ESI-TOF) m/z 517.1 [M+H⁺] with a purity of >99%.

### Propyl 4-(4-chloro-2-(4-(methylcarbamoyl)phenyl)quinoline-7-carbonyl)piperazine-1-carboxylate (B118)

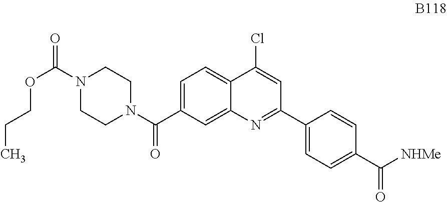 [SMILES: CCCOC(=O)N1CCN(C(=O)c2ccc3c(Cl)cc(-c4ccc(C(=O)NC)cc4)nc3c2)CC1]

Compound B118 was synthesized according to General Procedure L using propyl 4-(2,4-dichloroquinoline-7-carbonyl)piperazine-1-carboxylate and 4-(N-methylaminocarbonyl)phenylboronic acid as starting materials.

¹H NMR (400 MHz, DMSO-d₆) δ 8.59 (d, J=4.6 Hz, 1H), 8.56 (s, 1H), 8.42 (d, J=8.5 Hz, 2H), 8.30 (d, J=8.5 Hz, 1H), 8.18 (s, 1H), 8.03 (d, J=8.4 Hz, 2H), 7.78 (dd, J=8.6, 1.4 Hz, 1H), 3.98 (t, J=6.6 Hz, 2H), 3.81-3.36 (m, 8H), 2.83 (d, J=4.5 Hz, 3H), 1.59 (dd, J=13.9, 6.8 Hz, 2H), 0.89 (t, J=7.2 Hz, 3H).

LCMS (ESI-TOF) m/z 495.1 [M+H⁺] with a purity of >98%.

### Propyl (R)-4-(4-chloro-2-(4-((methylamino)methyl)phenyl)quinoline-7-carbonyl)-2-methylpiperazine-1-carboxylate (B119)

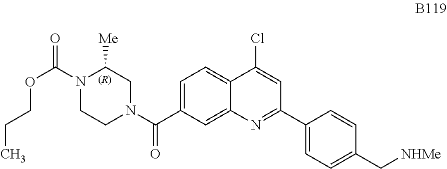

Step 1: Intermediate propyl (R)-4-(4-chloro-2-(4-formylphenyl)quinoline-7-carbonyl)-2-methylpiperazine-1-carboxylate was synthesized according to General Procedure L using propyl (R)-4-(2,4-dichloroquinoline-7-carbonyl)-2-methylpiperazine-1-carboxylate and 4-formylphenyl boronic acid as starting materials.

Step 2: To a solution of propyl (R)-4-(4-chloro-2-(4-formylphenyl)quinoline-7-carbonyl)-2-methylpiperazine-1-carboxylate (242 mg, 0.504 mmol) in methanol (5 mL) was added a 2 M solution of methylamine in tetrahydrofuran (0.52 mL, 1.04 mmol, 2.1 equiv). The mixture was cooled to 0° C. and sodium borohydride (40 mg, 1.06 mmol, 2.1 equiv) was added. After 15 min, the reaction was quenched by adding water (5 mL) and ethyl acetate (100 mL). The organic layer was separated and washed twice with water (50 mL) and thrice with brine (50 mL), dried over anhydrous sodium sulfate, filtered and concentrated. The crude material was purified by column chromatography to afford compound B119 as a yellow solid upon lyophilisation (100 mg, 40%).

¹H NMR (400 MHz, 80° C., DMSO-d₆) δ 8.36 (s, 1H), 8.27 (d, J=8.6 Hz, 1H), 8.23 (d, J=8.2 Hz, 2H), 8.09 (s, 1H), 7.70 (d, J=8.5 Hz, 1H), 7.50 (d, J=8.1 Hz, 2H), 4.40-3.77 (m, 6H), 3.74 (s, 2H), 3.39-3.10 (m, 3H), 2.33 (s, 3H), 2.00 (br s, 1H), 1.66-1.54 (m, 2H), 1.14 (d, J=6.3 Hz, 3H), 0.89 (t, J=7.4 Hz, 3H).

LCMS (ESI-TOF) m/z 495.2 [M+H⁺] with a purity of >99%.

### Propyl 4-(4-chloro-2-(4-methoxy-3-((methylamino)methyl)phenyl)quinoline-7-carbonyl)piperazine-1-carboxylate (B120)

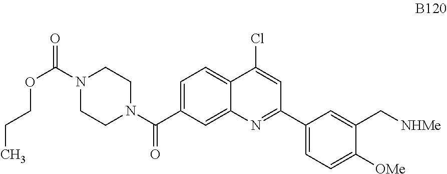 [SMILES: CCCOC(=O)N1CCN(C(=O)c2ccc3c(Cl)cc(-c4ccc(OC)c(CNC)c4)nc3c2)CC1]

In the synthesis of compound B100, a side product propyl 4-(4-chloro-2-(3-formyl-4-methoxyphenyl)quinoline-7-carbonyl)piperazine-1-carboxylate was isolated. A solution of this side product (20 mg, 0.0403 mmol) in methanol (2 mL) was added a 2 M solution of methylamine in tetrahydrofuran (0.05 mL, 0.1 mmol, 2.5 equiv). Upon cooling to 0° C., sodium borohydride (5 mg, 0.132 mmol, 3.3 equiv) was added. After 15 min, the reaction was quenched by adding water (1 mL) and ethyl acetate (50 mL). The organic layer was separated and washed twice with water (20 mL) and thrice with brine (20 mL), dried over anhydrous sodium sulfate, filtered and concentrated. The crude material was purified by column chromatography to afford compound B120 as a yellow solid upon lyophilisation (5 mg, 24%).

¹H NMR (400 MHz, 80° C., DMSO-d₆) δ 8.42 (s, 1H), 8.30 (d, J=5.8 Hz, 1H), 8.25 (d, J=8.6 Hz, 2H), 8.10 (s, 1H), 7.70 (d, J=8.5 Hz, 1H), 7.16 (d, J=8.6 Hz, 1H), 3.98 (t, J=6.6 Hz, 2H), 3.90 (s, 3H), 3.76 (s, 2H), 3.74-3.37 (m, 8H), 2.36 (s, 3H), 1.59 (d, J=6.9 Hz, 2H), 0.89 (t, J=7.3 Hz, 3H).

LCMS (ESI-TOF) m/z 511.2 [M+H⁺] with a purity of >99%.

### Propyl (R)-4-(2-(4-acetamidophenyl)-4-chloroquinoline-7-carbonyl)-2-methylpiperazine-1-carboxylate (B121)

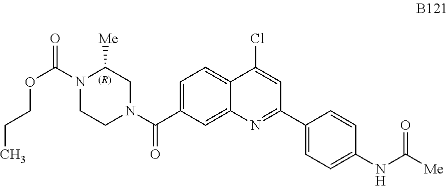

Compound B121 was synthesized according to General Procedure L using propyl (R)-4-(2,4-dichloroquinoline-7-carbonyl)-2-methylpiperazine-1-carboxylate and 4-acetylaminophenyl boronic acid as starting materials.

¹H NMR (400 MHz, 80° C., DMSO-d₆) δ 9.95 (s, 1H), 8.33 (s, 1H), 8.25 (dd, J=8.6, 3.7 Hz, 3H), 8.07 (s, 1H), 7.75 (d, J=8.7 Hz, 2H), 7.69 (d, J=8.5 Hz, 1H), 4.45-3.73 (m, 6H), 3.35-3.13 (m, 3H), 2.09 (s, 3H), 1.67-1.51 (m, 2H), 1.14 (d, J=5.9 Hz, 3H), 0.89 (t, J=7.4 Hz, 3H).

LCMS (ESI-TOF) m/z 509.1 [M+H⁺] with a purity of >98%.

### Propyl (R)-4-(2-(4-(aminomethyl)phenyl)-4-chloro-3-methylquinoline-7-carbonyl)-2-methylpiperazine-1-carboxylate (B122)

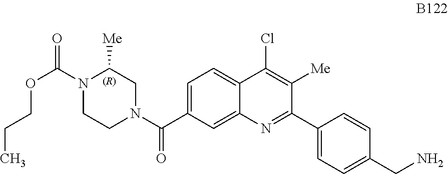

Compound B122 was synthesized according to General Procedure D using compound B114 as starting material.

¹H NMR (400 MHz, 80° C., DMSO-d₆) δ 8.27 (d, J=8.6 Hz, 1H), 8.01 (s, 1H), 7.71 (dd, J=8.6, 1.4 Hz, 1H), 7.54 (d, J=8.1 Hz, 2H), 7.48 (d, J=8.0 Hz, 2H), 4.40-3.66 (m, 8H), 3.39-3.16 (m, 3H), 2.52 (s, 3H), 1.66-1.52 (m, 2H), 1.12 (d, J=6.6 Hz, 3H), 0.89 (t, J=7.4 Hz, 3H).

LCMS (ESI-TOF) m/z 495.2 [M+H⁺] with a purity of >99%.

### Propyl 4-(4-chloro-2-(1-methyl-1H-indazol-5-yOquinoline-7-carbonyl)piperazine-1-carboxylate (B123)

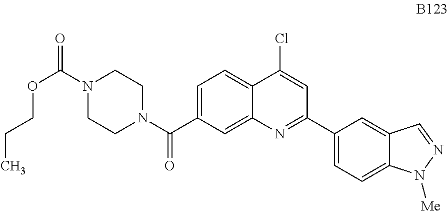 [SMILES: CCCOC(=O)N1CCN(C(=O)c2ccc3c(Cl)cc(-c4ccc5c(cnn5C)c4)nc3c2)CC1]

Compound B123 was synthesized according to General Procedure L using propyl 4-(2,4-dichloroquinoline-7-carbonyl)piperazine-1-carboxylate and 1-methylindazol-5-ylboronic acid as starting materials.

¹H NMR (400 MHz, DMSO-d₆) δ 8.78 (s, 1H), 8.57 (s, 1H), 8.45 (dd, J=8.9, 1.6 Hz, 1H), 8.27 (d, J=8.5 Hz, 1H), 8.22 (s, 1H), 8.14 (s, 1H), 7.82 (d, J=8.9 Hz, 1H), 7.73 (dd, J=8.5, 1.5 Hz, 1H), 4.11 (s, 3H), 3.98 (t, J=6.5 Hz, 2H), 3.82-3.38 (m, 8H), 1.59 (dd, J=13.9, 6.8 Hz, 2H), 0.89 (t, J=7.2 Hz, 3H).

LCMS (ESI-TOF) m/z 492.1 [M+H⁺] with a purity of >99%.

### Propyl (R)-4-(4-chloro-2-(3-((methylamino)methyl)phenyl)quinoline-7-carbonyl)-2-methylpiperazine-1-carboxylate (B124)

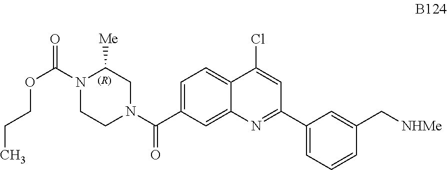

Step 1: Step 1: Intermediate propyl (R)-4-(4-chloro-2-(3-formylphenyl)quinoline-7-carbonyl)-2-methylpiperazine-1-carboxylate was synthesized according to General Procedure L using propyl (R)-4-(2,4-dichloroquinoline-7-carbonyl)-2-methylpiperazine-1-carboxylate and 3-formylphenyl boronic acid as starting materials.

Step 2: To a solution of propyl (R)-4-(4-chloro-2-(3-formylphenyl)quinoline-7-carbonyl)-2-methylpiperazine-1-carboxylate (350 mg, 0.729 mmol) in methanol (5 mL) was added a 2 M solution of methylamine in tetrahydrofuran (0.73 mL, 1.46 mmol, 2 equiv). The mixture was cooled to 0° C. and sodium borohydride (55 mg, 1.46 mmol, 2 equiv) was added. After 15 min, the reaction was quenched by adding water (5 mL) and ethyl acetate (100 mL). The organic layer was separated and washed twice with water (50 mL) and thrice with brine (50 mL), dried over anhydrous sodium sulfate, filtered and concentrated. The crude material was purified by column chromatography to afford compound B124 as a yellow solid upon lyophilisation (150 mg, 42%).

¹H NMR (400 MHz, 80° C., DMSO-d₆) δ 8.37 (s, 1H), 8.28 (d, J=8.5 Hz, 1H), 8.23 (s, 1H), 8.14 (d, J=6.3 Hz, 1H), 8.12 (s, 1H), 7.72 (dd, J=8.5, 1.4 Hz, 1H), 7.55-7.43 (m, 2H), 4.43-3.68 (m, 8H), 3.39-3.10 (m, 3H), 2.34 (s, 3H), 2.06 (br s, 1H), 1.69-1.52 (m, 2H), 1.14 (d, J=5.6 Hz, 3H), 0.89 (t, J=7.4 Hz, 3H).

LCMS (ESI-TOF) m/z 495.2 [M+H⁺] with a purity of >99%.

### Propyl 4-(4-chloro-2-cyclopropylquinoline-7-carbonyl)piperazine-1-carboxylate (B125)

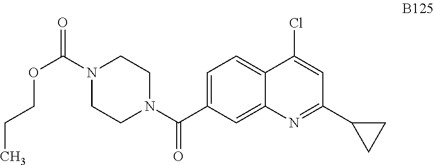 [SMILES: CCCOC(=O)N1CCN(C(=O)c2ccc3c(Cl)cc(C4CC4)nc3c2)CC1]

Compound B125 was synthesized according to General Procedure L using propyl 4-(2,4-dichloroquinoline-7-carbonyl)piperazine-1-carboxylate and cyclopropylboronic acid as starting materials.

¹H NMR (400 MHz, DMSO-d₆) δ 8.18 (d, J=8.5 Hz, 1H), 7.90 (s, 1H), 7.80 (s, 1H), 7.63 (dd, J=8.5, 1.3 Hz, 1H), 3.97 (t, J=6.6 Hz, 2H), 3.76-3.34 (m, 8H), 2.39-2.29 (m, 1H), 1.58 (dd, J=14.0, 7.0 Hz, 2H), 1.17-1.05 (m, 4H), 0.93-0.79 (m, 3H).

LCMS (ESI-TOF) m/z 402.1 [M+H⁺] with a purity of >99%.

### Propyl 4-(4-chloro-2-(5-((dimethylamino)methyl)thiophen-2-yl)quinoline-7-carbonyl)piperazine-1-carboxylate (B126)

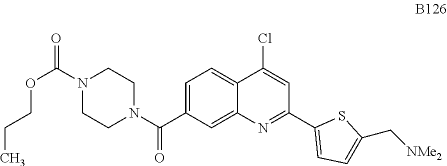 [SMILES: CCCOC(=O)N1CCN(C(=O)c2ccc3c(Cl)cc(-c4ccc(CN(C)C)s4)nc3c2)CC1]

Compound B126 was synthesized according to General Procedure L using propyl 4-(2,4-dichloroquinoline-7-carbonyl)piperazine-1-carboxylate and 5-((dimethylamino)methyl)thiophen-2-ylboronic acid, pinacol ester as starting materials.

¹H NMR (400 MHz, 80° C., DMSO-d₆) δ 8.29 (s, 1H), 8.21 (d, J=8.5 Hz, 1H), 7.97 (d, J=1.2 Hz, 1H), 7.90 (d, J=3.7 Hz, 1H), 7.66 (dd, J=8.5, 1.5 Hz, 1H), 7.04 (d, J=3.7 Hz, 1H), 3.99 (t, J=6.6 Hz, 2H), 3.66 (s, 2H), 3.58-3.41 (m, 8H), 2.25 (s, 6H), 1.69-1.51 (m, 2H), 0.90 (t, J=7.4 Hz, 3H).

LCMS (ESI-TOF) m/z 501.1 [M+H⁺] with a purity of >98%.

### Propyl 4-(4-chloro-2-(2-methyl-2H-indazol-5-yl)quinoline-7-carbonyl)piperazine-1-carboxylate (B127)

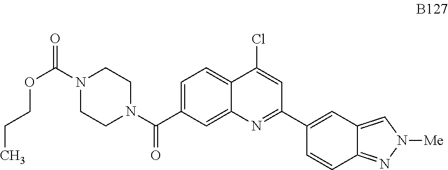 [SMILES: CCCOC(=O)N1CCN(C(=O)c2ccc3c(Cl)cc(-c4ccc5nn(C)cc5c4)nc3c2)CC1]

Compound B127 was synthesized according to General Procedure L using propyl 4-(2,4-dichloroquinoline-7-carbonyl)piperazine-1-carboxylate and 2-methylindazol-5-ylboronic acid as starting materials.

¹H NMR (400 MHz, DMSO-d₆) δ 8.74 (s, 1H), 8.54 (s, 2H), 8.33-8.24 (m, 2H), 8.13 (s, 1H), 7.73 (t, J=9.3 Hz, 2H), 4.22 (s, 3H), 3.98 (t, J=6.5 Hz, 2H), 3.80-3.38 (m, 8H), 1.69-1.51 (m, 2H), 0.89 (t, J=7.1 Hz, 3H).

LCMS (ESI-TOF) m/z 492.1 [M+H⁺] with a purity of >97%.

### Propyl 4-(4-chloro-2-(1H-indazol-5-yl)quinoline-7-carbonyl)piperazine-1-carboxylate (B128)

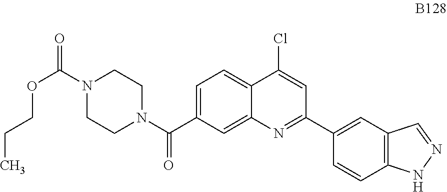 [SMILES: CCCOC(=O)N1CCN(C(=O)c2ccc3c(Cl)cc(-c4ccc5[nH]ncc5c4)nc3c2)CC1]

Compound B128 was synthesized according to General Procedure L using propyl 4-(2,4-dichloroquinoline-7-carbonyl)piperazine-1-carboxylate and 1H-indazole-5-boronic acid as starting materials.

¹H NMR (400 MHz, DMSO-d₆) δ 13.29 (s, 1H), 8.78 (s, 1H), 8.55 (s, 1H), 8.41 (d, J=9.0 Hz, 1H), 8.27 (d, J=8.5 Hz, 1H), 8.24 (s, 1H), 8.13 (s, 1H), 7.71 (t, J=8.5 Hz, 2H), 3.98 (t, J=6.5 Hz, 2H), 3.78-3.38 (m, 8H), 1.67-1.47 (m, 2H), 0.89 (t, J=7.2 Hz, 3H).

LCMS (ESI-TOF) m/z 478.1 [M+H⁺] with a purity of >98%.

### Propyl 4-(4-chloro-2-(1H-indazol-6-yl)quinoline-7-carbonyl)piperazine-1-carboxylate (B129)

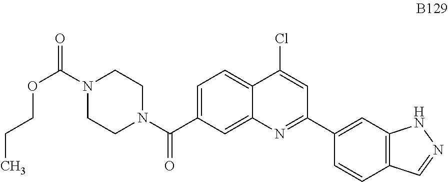 [SMILES: CCCOC(=O)N1CCN(C(=O)c2ccc3c(Cl)cc(-c4ccc5cn[nH]c5c4)nc3c2)CC1]

Compound B129 was synthesized according to General Procedure L using propyl 4-(2,4-dichloroquinoline-7-carbonyl)piperazine-1-carboxylate and 1H-indazole-6-boronic acid as starting materials.

¹H NMR (400 MHz, DMSO-d₆) δ 13.38 (s, 1H), 8.59 (s, 1H), 8.50 (s, 1H), 8.29 (d, J=8.5 Hz, 1H), 8.18 (s, 2H), 8.11 (d, J=8.5 Hz, 1H), 7.94 (d, J=8.6 Hz, 1H), 7.76 (d, J=8.6 Hz, 1H), 3.98 (t, J=6.5 Hz, 2H), 3.80-3.36 (m, 8H), 1.68-1.54 (m, 2H), 0.89 (t, J=7.1 Hz, 3H).

LCMS (ESI-TOF) m/z 478.1 [M+H⁺] with a purity of >96%.

### Propyl 4-(4-chloro-2-(1-methyl-1H-pyrrol-3-yl)quinoline-7-carbonyl)piperazine-1-carboxylate (B130)

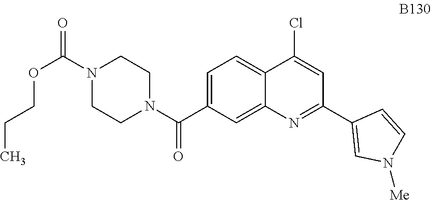 [SMILES: CCCOC(=O)N1CCN(C(=O)c2ccc3c(Cl)cc(-c4ccn(C)c4)nc3c2)CC1]

Compound B130 was synthesized according to General Procedure L using propyl 4-(2,4-dichloroquinoline-7-carbonyl)piperazine-1-carboxylate and 1-methylpyrrole-3-boronic acid, pinacol ester as starting materials.

¹H NMR (400 MHz, DMSO-d₆) δ 8.14 (d, J=8.5 Hz, 1H), 8.07 (s, 1H), 7.91 (d, J=1.0 Hz, 1H), 7.70 (s, 1H), 7.58 (dd, J=8.5, 1.4 Hz, 1H), 6.82 (dt, J=4.4, 2.6 Hz, 2H), 3.98 (t, J=6.6 Hz, 2H), 3.71 (s, 3H), 3.69-3.34 (m, 8H), 1.64-1.50 (m, 2H), 0.89 (t, J=7.3 Hz, 3H).

LCMS (ESI-TOF) m/z 441.1 [M+H⁺] with a purity of >96%.

Propyl (R)-4-(4-chloro-2-(2-methoxypyridin-4-yl)quinoline-7-carbonyl)-2-methylpiperazine-1-carboxylate (B131)

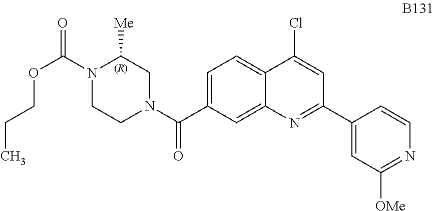

Compound B131 was synthesized according to General Procedure L using propyl (R)-4-(2,4-dichloroquinoline-7-carbonyl)-2-methylpiperazine-1-carboxylate and 2-methoxypyridin-4-ylboronic acid as starting materials.

¹H NMR (400 MHz, 80° C., DMSO-d₆) δ 8.48 (s, 1H), 8.35 (d, J=5.4 Hz, 1H), 8.32 (d, J=8.6 Hz, 1H), 8.16 (s, 1H), 7.83 (dd, J=5.4, 1.4 Hz, 1H), 7.81-7.75 (m, 1H), 7.65 (s, 1H), 4.45-3.65 (m, 9H), 3.36-3.09 (m, 3H), 1.65-1.52 (m, 2H), 1.14 (d, J=6.2 Hz, 3H), 0.89 (t, J=7.4 Hz, 3H).

LCMS (ESI-TOF) m/z 483.1 [M+H⁺] with a purity of >98%.

### Propyl (R)-4-(4-chloro-2-(1H-pyrrolo[2,3-b]pyridin-5-yl)quinoline-7-carbonyl)-2-methylpiperazine-1-carboxylate (B132)

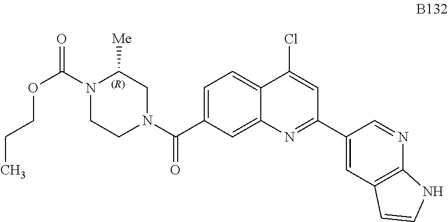

Compound B132 was synthesized according to General Procedure L using propyl (R)-4-(2,4-dichloroquinoline-7-carbonyl)-2-methylpiperazine-1-carboxylate and 1H-pyrrolo[2,3-b]pyridin-5-ylboronic acid as starting materials.

¹H NMR (400 MHz, 80° C., DMSO-d₆) δ 11.67 (s, 1H), 9.17 (d, J=2.0 Hz, 1H), 8.84 (d, J=2.0 Hz, 1H), 8.47 (s, 1H), 8.27 (d, J=8.5 Hz, 1H), 8.11 (s, 1H), 7.70 (d, J=8.5 Hz, 1H), 7.52 (d, J=3.4 Hz, 1H), 6.59 (d, J=3.4 Hz, 1H), 4.48-3.59 (m, 6H), 3.41-3.09 (m, 3H), 1.69-1.52 (m, 2H), 1.15 (d, J=6.2 Hz, 3H), 0.90 (t, J=7.4 Hz, 3H).

LCMS (ESI-TOF) m/z 492.1 [M+H⁺] with a purity of >96%.

### Propyl 4-(4-chloro-2-(1-methylcyclopropyl)quinoline-7-carbonyl)piperazine-1-carboxylate (B133)

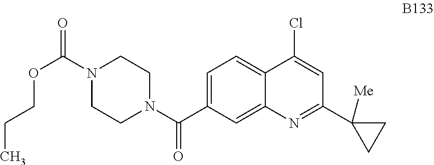 [SMILES: CCCOC(=O)N1CCN(C(=O)c2ccc3c(Cl)cc(C4(C)CC4)nc3c2)CC1]

Compound B133 was synthesized according to General Procedure C1 using intermediate 4-chloro-2-(1-methylcyclopropyl)quinoline-7-carboxylic acid in the synthesis of compound B109 and n-propyl piperazine-1-carboxylate as starting materials.

¹H NMR (400 MHz, CDC1₃) δ 8.21 (d, J=8.4 Hz, 1H), 7.99 (s, 1H), 7.55 (d, J=10.8 Hz, 1H), 7.54 (s, 1H), 4.08 (t, J=6.4 Hz, 2H), 3.80-3.50 (m, 8H), 1.67 (dd, J=7.2 Hz, 2H), 1.61 (s, 3H), 1.40-1.38 (m, 2H), 0.97-0.92 (m, 5H).

LCMS (ESI-TOF) m/z 416.2 [M+H⁺] with a purity of >99%.

### Propyl 4-(2-(4-(1H-pyrazol-1-yl)phenyl)-4-chloroquinoline-7-carbonyl)piperazine-1-carboxylate (B134)

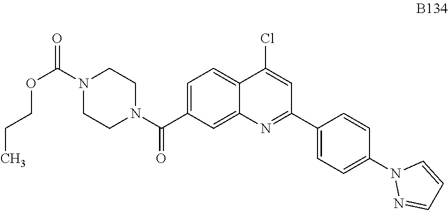 [SMILES: CCCOC(=O)N1CCN(C(=O)c2ccc3c(Cl)cc(-c4ccc(-n5cccn5)cc4)nc3c2)CC1]

Compound B134 was synthesized according to General Procedure I, K and C1 using 4′-(1H-pyrazol-1-yl)acetophenone (General Procedure I) and n-propyl piperazine-1-carboxylate (General Procedure C1) as starting materials.

¹H NMR (400 MHz, DMSO-d₆) δ 8.66 (d, J=2.4 Hz, 1H), 8.56 (s, 1H), 8.49 (d, J=8.7 Hz, 2H), 8.29 (d, J=8.5 Hz, 1H), 8.16 (s, 1H), 8.07 (d, J=8.7 Hz, 2H), 7.83 (s, 1H), 7.76 (d, J=8.5 Hz, 1H), 6.62 (s, 1H), 3.98 (t, J=6.6 Hz, 2H), 3.78-3.35 (m, 8H), 1.59 (dd, J=13.8, 6.8 Hz, 2H), 0.89 (t, J=7.3 Hz, 3H).

LCMS (ESI-TOF) m/z 504.1 [M+H⁺] with a purity of >97%.

### Propyl 4-(2-(4-(1H-pyrazol-5-yl)phenyl)-4-chloroquinoline-7-carbonyl)piperazine-1-carboxylate (B135)

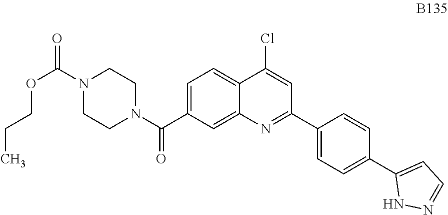 [SMILES: CCCOC(=O)N1CCN(C(=O)c2ccc3c(Cl)cc(-c4ccc(-c5ccn[nH]5)cc4)nc3c2)CC1]

Compound B135 was synthesized according to General Procedure L using propyl 4-(2,4-dichloroquinoline-7-carbonyl)piperazine-1-carboxylate and [4-(1H-pyrazol-5-yl)phenyl]boronic acid as starting materials.

¹H NMR (400 MHz, 80° C., DMSO-d₆) δ 12.86 (br s, 1H), 8.42 (s, 1H), 8.35 (d, J=8.2 Hz, 2H), 8.27 (d, J=8.5 Hz, 1H), 8.14 (d, J=1.1 Hz, 1H), 7.98 (d, J=8.0 Hz, 2H), 7.72 (dd, J=8.5, 1.5 Hz, 2H), 6.78 (d, J=1.9 Hz, 1H), 4.00 (t, J=6.6 Hz, 2H), 3.69-3.37 (m, 8H), 1.68-1.51 (m, 2H), 0.90 (t, J=7.4 Hz, 3H).

LCMS (ESI-TOF) m/z 504.1 [M+H⁺] with a purity of >97%.

### Propyl 4-(2-(4-(1H-pyrazol-4-yl)phenyl)-4-chloroquinoline-7-carbonyl)piperazine-1-carboxylate (B136)

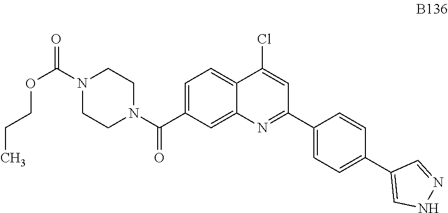 [SMILES: CCCOC(=O)N1CCN(C(=O)c2ccc3c(Cl)cc(-c4ccc(-c5cn[nH]c5)cc4)nc3c2)CC1]

Compound B136 was synthesized according to General Procedure L using propyl 4-(2,4-dichloroquinoline-7-carbonyl)piperazine-1-carboxylate and 4-[4-(4,4,5,5-tetramethyl-[1,3,2]dioxaborolan-2-yl)phenyl]-1H-pyrazole as starting materials.

¹H NMR (400 MHz, 80° C., DMSO-d₆) δ 12.85 (s, 1H), 8.39 (s, 1H), 8.29 (d, J=8.4 Hz, 2H), 8.26 (d, J=8.5 Hz, 1H), 8.23-7.90 (m, 3H), 7.78 (d, J=8.4 Hz, 2H), 7.71 (dd, J=8.5, 1.5 Hz, 1H), 4.00 (t, J=6.6 Hz, 2H), 3.65-3.42 (m, 8H), 1.67-1.49 (m, 2H), 0.90 (t, J=7.4 Hz, 3H).

LCMS (ESI-TOF) m/z 504.1 [M+H⁺] with a purity of >97%.

### 4-(4-Chloro-7-(4-(propoxycarbonyl)piperazine-1-carbonyl)quinolin-2-yl)-2-methylbenzoic acid (B137)

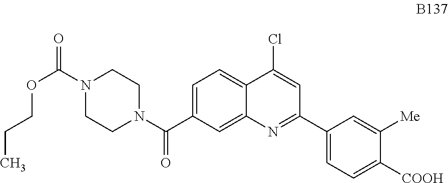 [SMILES: CCCOC(=O)N1CCN(C(=O)c2ccc3c(Cl)cc(-c4ccc(C(=O)O)c(C)c4)nc3c2)CC1]

Compound B137 was synthesized according to General Procedure L using propyl 4-(2,4-dichloroquinoline-7-carbonyl)piperazine-1-carboxylate and 4-borono-2-methylbenzoic acid as starting materials.

¹H NMR (400 MHz, DMSO-d₆) δ 13.04 (br s, 1H), 8.55 (s, 1H), 8.30 (d, J=8.6 Hz, 1H), 8.26 (s, 1H), 8.23 (d, J=8.6 Hz, 1H), 8.18 (s, 1H), 7.97 (d, J=8.2 Hz, 1H), 7.77 (d, J=8.7 Hz, 1H), 3.98 (t, J=6.5 Hz, 2H), 3.80-3.36 (m, 8H), 2.65 (s, 3H), 1.70-1.45 (m, 2H), 0.89 (t, J=7.1 Hz, 3H).

LCMS (ESI-TOF) m/z 496.1 [M+H⁺] with a purity of >99%.

### Propyl 4-(4-chloro-2-(1H-pyrazol-5-yl)quinoline-7-carbonyl)piperazine-1-carboxylate (B138)

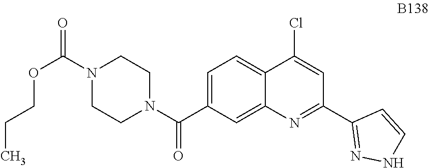 [SMILES: CCCOC(=O)N1CCN(C(=O)c2ccc3c(Cl)cc(-c4cc[nH]n4)nc3c2)CC1]

Compound B138 was synthesized according to General Procedure L using propyl 4-(2,4-dichloroquinoline-7-carbonyl)piperazine-1-carboxylate and 1H-pyrazole-3-boronic acid as starting materials.

¹H NMR (400 MHz, DMSO-d₆) δ 13.35 (br s, 1H), 8.34 (s, 1H), 8.26 (d, J=8.5 Hz, 1H), 8.08 (s, 1H), 7.92 (br s, 1H), 7.72 (d, J=8.5 Hz, 1H), 7.04 (br s, 1H), 3.98 (t, J=6.5 Hz, 2H), 3.81-3.34 (m, 8H), 1.59 (dd, J=13.8, 6.7 Hz, 2H), 0.89 (t, J=7.1 Hz, 3H).

LCMS (ESI-TOF) m/z 428.1 [M+H⁺] with a purity of >96%.

### Propyl 4-(2-(1H-benzo[d]imidazol-5-yl)-4-chloroquinoline-7-carbonyl)piperazine-1-carboxylate (B139)

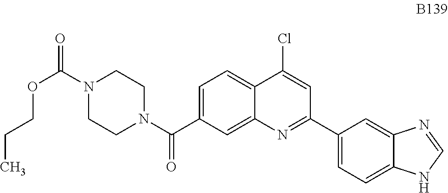 [SMILES: CCCOC(=O)N1CCN(C(=O)c2ccc3c(Cl)cc(-c4ccc5[nH]cnc5c4)nc3c2)CC1]

Compound B139 was synthesized according to General Procedure L using propyl 4-(2,4-dichloroquinoline-7-carbonyl)piperazine-1-carboxylate and 1H-benzimidazole-5-boronic acid as starting materials.

¹H NMR (400 MHz, DMSO-d₆) δ 12.69 (br s, 1H), 8.59 (s, 1H), 8.55 (s, 1H), 8.34 (s, 1H), 8.26 (t, J=8.8 Hz, 2H), 8.14 (s, 1H), 7.73 (t, J=8.3 Hz, 2H), 3.98 (t, J=6.5 Hz, 2H), 3.80-3.36 (m, 8H), 1.59 (dd, J=13.8, 6.9 Hz, 2H), 0.89 (t, J=7.1 Hz, 3H).

LCMS (ESI-TOF) m/z 478.1 [M+H⁺] with a purity of >99%.

Propyl 4-(4-chloro-2-(1-methyl-1H-indo1-5-yl)quinoline-7-carbonyl)piperazine-1-carboxylate (B140)

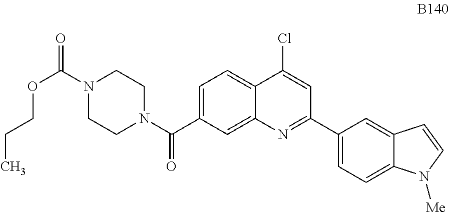 [SMILES: CCCOC(=O)N1CCN(C(=O)c2ccc3c(Cl)cc(-c4ccc5c(ccn5C)c4)nc3c2)CC1]

Compound B140 was synthesized according to General Procedure L using propyl 4-(2,4-dichloroquinoline-7-carbonyl)piperazine-1-carboxylate and 1-methylindole-5-boronic acid, pinacol ester as starting materials.

¹H NMR (400 MHz, DMSO-d₆) 6 8.58 (s, 1H), 8.51 (s, 1H), 8.24 (d, J=8.5 Hz, 1H), 8.21 (dd, J=8.7, 1.4 Hz, 1H), 8.11 (s, 1H), 7.69 (dd, J=8.5, 1.3 Hz, 1H), 7.61 (d, J=8.7 Hz, 1H), 7.43 (d, J=3.0 Hz, 1H), 6.59 (d, J=3.0 Hz, 1H), 3.98 (t, J=6.6 Hz, 2H), 3.86 (s, 3H), 3.78-3.35 (m, 8H), 1.59 (dd, J=13.9, 6.9 Hz, 2H), 0.89 (t, J=7.3 Hz, 3H).

LCMS (ESI-TOF) m/z 491.1 [M+H⁺] with a purity of >99%.

### Propyl 4-(4-chloro-2-(1H-indazol-4-yl)quinoline-7-carbonyl)piperazine-1-carboxylate (B141)

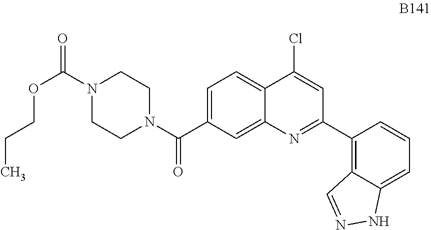 [SMILES: CCCOC(=O)N1CCN(C(=O)c2ccc3c(Cl)cc(-c4cccc5[nH]ncc45)nc3c2)CC1]

Compound B141 was synthesized according to General Procedure L using propyl 4-(2,4-dichloroquinoline-7-carbonyl)piperazine-1-carboxylate and 1H-indazol-4-ylboronic acid, pinacol ester as starting materials.

¹H NMR (400 MHz, DMSO-d₆) δ 13.30 (s, 1H), 9.05 (s, 1H), 8.58 (s, 1H), 8.34 (s, 1H), 8.31 (d, J=8.5 Hz, 1H), 8.03 (d, J=7.3 Hz, 1H), 7.76 (t, J=9.6 Hz, 2H), 7.53 (t, J=7.8 Hz, 1H), 3.98 (t, J=6.6 Hz, 2H), 3.82-3.36 (m, 8H), 1.63-1.50 (m, 2H), 0.89 (t, J=6.8 Hz, 3H).

LCMS (ESI-TOF) m/z 478.1 [M+H⁺] with a purity of >99%.

### Propyl (R)-4-(4-chloro-2-(3-(hydroxymethyl)phenyl)quinoline-7-carbonyl)-2-methylpiperazine-1-carboxylate (B142)

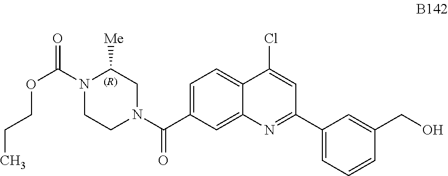

Compound B142 was synthesized according to General Procedure L using propyl (R)-4-(2,4-dichloroquinoline-7-carbonyl)-2-methylpiperazine-1-carboxylate and 3-(hydroxymethyl)phenylboronic acid as starting materials.

NMR (400 MHz, 80° C., DMSO-d₆) δ 8.35 (s, 1H), 8.28 (d, J=8.4 Hz, 1H), 8.25 (s, 1H), 8.14 (d, J=6.8 Hz, 1H), 8.12 (s, 1H), 7.72 (d, J=8.4 Hz, 1H), 7.55-7.46 (m, 2H), 5.06 (t, J=5.7 Hz, 1H), 4.64 (d, J=5.6 Hz, 2H), 4.37-3.73 (m, 6H), 3.18 (d, J=4.8 Hz, 3H), 1.68-1.51 (m, 2H), 1.14 (d, J=6.2 Hz, 3H), 0.89 (t, J=7.4 Hz, 3H).

LCMS (ESI-TOF) m/z 482.1 [M+H⁺] with a purity of >99%.

### Propyl 4-(4-chloro-2-(3-hydroxyphenyl)quinoline-7-carbonyl)piperazine-1-carboxylate (B143)

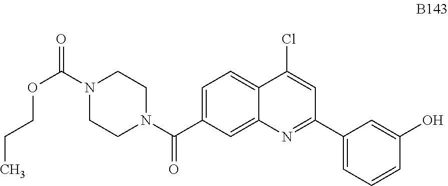 [SMILES: CCCOC(=O)N1CCN(C(=O)c2ccc3c(Cl)cc(-c4cccc(O)c4)nc3c2)CC1]

Compound B143 was synthesized according to General Procedure L using propyl 4-(2,4-dichloroquinoline-7-carbonyl)piperazine-1-carboxylate and 3-hydroxyphenylboronic acid as starting materials.

¹H NMR (400 MHz, 80° C., DMSO-d₆) δ 8.28 (d, J=7.9 Hz, 2H), 8.12 (s, 1H), 7.76-7.66 (m, 3H), 7.37 (t, J=7.9 Hz, 1H), 6.96 (dd, J=7.7, 1.9 Hz, 1H), 4.01 (t, J=6.6 Hz, 2H), 3.69-3.36 (m, 8H), 1.68-1.55 (m, 2H), 0.91 (t, J=7.4 Hz, 3H).

LCMS (ESI-TOF) m/z 454.1 [M+H⁺] with a purity of >99%.

### Propyl (S)-4-(4-chloro-2-(1-methyl-1H-indazol-5-yl)quinoline-7-carbonyl)-2-methylpiperazine-1-carboxylate (B144)

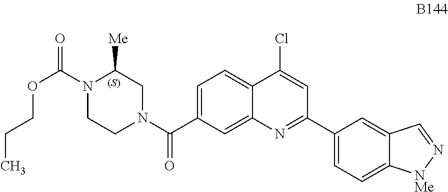

Compound B144 was synthesized according to General Procedure L using propyl (S)-4-(2,4-dichloroquinoline-7-carbonyl)-2-methylpiperazine-1-carboxylate and 1-methylindazol-5-ylboronic acid as starting materials.

¹H NMR (400 MHz, CDCl₃) δ 8.52 (s, 1H), 8.30 (d, J=8.8 Hz, 2H), 8.18 (s, 1H), 8.11 (d, J=2.4 Hz, 2H), 7.64 (d, J=8.4 Hz, 1H), 7.54 (d, J=8.8 Hz, 1H), 4.71-4.26 (m, 2H), 4.14 (s, 3H), 4.11-4.03 (m, 2H), 3.97-3.02 (m, 5H), 1.72-1.56 (m, 2H), 1.23 (br d, J=66 Hz, 3H), 0.95 (t, J=7.6 Hz, 3H).

LCMS (ESI-TOF) m/z 506.6 [M+H⁺] with a purity of >98%.

### Propyl (S)-4-(2-(3-(aminomethyl)phenyl)-4-chloroquinoline-7-carbonyl)-2-methylpiperazine-1-carboxylate (B145)

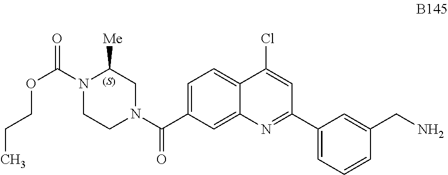

Compound B145 was synthesized according to General Procedure L using propyl (S)-4-(2,4-dichloroquinoline-7-carbonyl)-2-methylpiperazine-1-carboxylate and 3-(aminomethyl)phenylboronic acid hydrochloride salt as starting materials.

¹H NMR (400 MHz, DMSO-d₆) δ 8.49 (s, 1H), 8.30-8.28 (m, 2H), 8.19-8.11 (m, 2H), 7.75 (br s, 1H), 7.52 (d, J=4.8 Hz, 2H), 4.49-3.53 (m, 8H), 3.22-2.97 (m, 3H), 1.63-1.54 (m, 2H), 1.12 (br d, J=70 Hz, 3H), 0.89 (t, J=7.2 Hz, 3H).

LCMS (ESI-TOF) m/z 481.3 [M+H⁺] with a purity of >97%.

### Propyl (S)-4-(2-(4-carbamoylphenyl)-4-chloroquinoline-7-carbonyl)-2-methylpiperazine-1-carboxylate (B146)

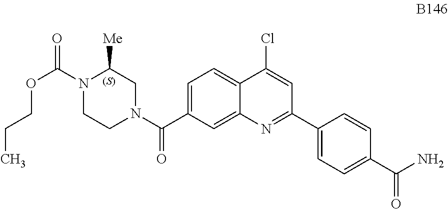

Compound B146 was synthesized according to General Procedure L using propyl (S)-4-(2,4-dichloroquinoline-7-carbonyl)-2-methylpiperazine-1-carboxylate and 4-(aminocarbonyl)phenylboronic acid as starting materials.

¹H NMR (400 MHz, DMSO-d₆) δ 8.57 (s, 1H), 8.42 (d, J=7.6 Hz, 2H), 8.31 (d, J=8.0 Hz, 1H), 8.18-8.06 (m, 4H), 7.78 (br s, 1H), 7.49 (s, 1H), 4.38-3.44 (m, 7H), 3.18-2.99 (m, 2H), 1.63-1.54 (m, 2H), 1.13 (br d, J=68 Hz, 3H), 0.89 (t, J=8.0 Hz, 3H).

LCMS (ESI-TOF) m/z 495.3 [M+H⁺] with a purity of >96%.

### Propyl 4-(4-chloro-2-(1H-pyrazol-4-yl)quinoline-7-carbonyl)piperazine-1-carboxylate (B147)

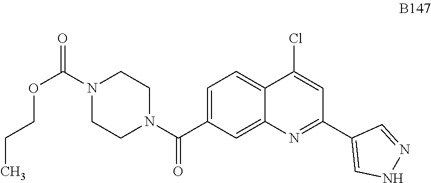 [SMILES: CCCOC(=O)N1CCN(C(=O)c2ccc3c(Cl)cc(-c4cn[nH]c4)nc3c2)CC1]

Compound B147 was synthesized according to General Procedure L using propyl 4-(2,4-dichloroquinoline-7-carbonyl)piperazine-1-carboxylate and pyrazole-4-boronic acid pinacol ester as starting materials.

¹H NMR (400 MHz, 80° C., DMSO-d₆) δ 13.10 (br s, 1H), 8.38 (br s, 2H), 8.19 (d, J=8.5 Hz, 1H), 8.16 (s, 1H), 7.98 (s, 1H), 7.63 (dd, J=8.6, 1.3 Hz, 1H), 3.99 (t, J=6.6 Hz, 2H), 3.67-3.35 (m, 8H), 1.65-1.52 (m, 2H), 0.90 (t, J=7.4 Hz, 3H).

LCMS (ESI-TOF) m/z 428.1 [M+H⁺] with a purity of >95%.

### Propyl 4-(4-chloro-2-(thiophen-3-yl)quinoline-7-carbonyl)piperazine-1-carboxylate (B148)

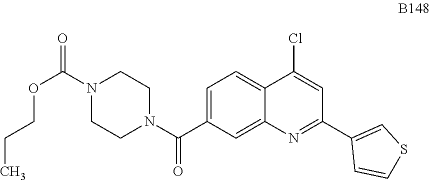 [SMILES: CCCOC(=O)N1CCN(C(=O)c2ccc3c(Cl)cc(-c4ccsc4)nc3c2)CC1]

Compound B148 was synthesized according to General Procedure L using propyl 4-(2,4-dichloroquinoline-7-carbonyl)piperazine-1-carboxylate and thiophene-3-boronic acid as starting materials.

¹H NMR (400 MHz, DMSO-d₆) δ 8.57 (d, J=1.9 Hz, 1H), 8.44 (s, 1H), 8.25 (d, J=8.5 Hz, 1H), 8.07 (s, 1H), 7.98 (d, J=5.0 Hz, 1H), 7.78-7.67 (m, 2H), 3.98 (t, J=6.6 Hz, 2H), 3.76-3.36 (m, 8H), 1.59 (dd, J=13.9, 6.7 Hz, 2H), 0.89 (t, J=7.2 Hz, 3H).

LCMS (ESI-TOF) m/z 444.0 [M+H⁺] with a purity of >98%.

### Propyl 4-(2-(6-acetamidopyridin-3-yl)-4-chloroquinoline-7-carbonyl)piperazine-1-carboxylate (B149)

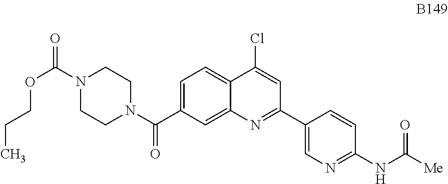 [SMILES: CCCOC(=O)N1CCN(C(=O)c2ccc3c(Cl)cc(-c4ccc(NC(C)=O)nc4)nc3c2)CC1]

Compound B149 was synthesized according to General Procedure L using propyl 4-(2,4-dichloroquinoline-7-carbonyl)piperazine-1-carboxylate and 2-acetamidopyridine-5-boronic acid as starting materials.

¹H NMR (400 MHz, DMSO-d₆) δ 10.80 (s, 1H), 9.26 (s, 1H), 8.71-8.64 (m, 1H), 8.54 (s, 1H), 8.27 (t, J=9.0 Hz, 2H), 8.14 (s, 1H), 7.75 (d, J=8.6 Hz, 1H), 3.98 (t, J=6.5 Hz, 2H), 3.75-3.38 (m, 8H), 2.15 (s, 3H), 1.59 (dd, J=12.1, 5.0 Hz, 2H), 0.89 (t, J=7.3 Hz, 3H).

LCMS (ESI-TOF) m/z 496.1 [M+H⁺] with a purity of >99%.

Propyl 4-(4-chloro-2-(4-fluoro-3-(hydroxymethyl)phenyBquinoline-7-carbonyl)piperazine-1-carboxylate (B150)

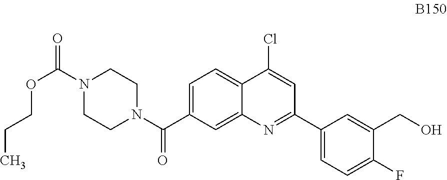 [SMILES: CCCOC(=O)N1CCN(C(=O)c2ccc3c(Cl)cc(-c4ccc(F)c(CO)c4)nc3c2)CC1]

Compound B150 was synthesized according to General Procedure L using propyl 4-(2,4-dichloroquinoline-7-carbonyl)piperazine-1-carboxylate and 4-fluoro-3-(hydroxymethyl)phenylboronic acid as starting materials.

¹H NMR (400 MHz, DMSO-d₆) δ 8.50-8.44 (m, 2H), 8.31-8.23 (m, 2H), 8.15 (s, 1H), 7.75 (d, J=8.5 Hz, 1H), 7.35 (t, J=9.2 Hz, 1H), 5.41 (t, J=5.3 Hz, 1H), 4.66 (d, J=4.7 Hz, 2H), 3.98 (t, J=6.6 Hz, 2H), 3.79-3.36 (m, 8H), 1.59 (dd, J=13.8, 7.0 Hz, 2H), 0.89 (t, J=7.1 Hz, 3H).

LCMS (ESI-TOF) m/z 486.1 [M+H⁺] with a purity of >99%.

### Propyl 4-(2-(3-carbamoyl-4-fluorophenyl)-4-chloroquinoline-7-carbonyl)piperazine-1-carboxylate (B151)

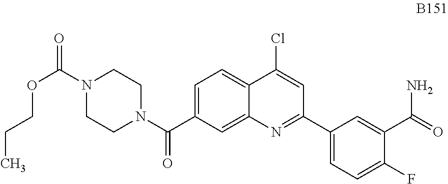 [SMILES: CCCOC(=O)N1CCN(C(=O)c2ccc3c(Cl)cc(-c4ccc(F)c(C(N)=O)c4)nc3c2)CC1]

Compound B151 was synthesized according to General Procedure L using propyl 4-(2,4-dichloroquinoline-7-carbonyl)piperazine-1-carboxylate and 3-(aminocarbonyl)-4-fluorophenylboronic acid as starting materials.

¹H NMR (400 MHz, DMSO-d₆) δ 8.60 (dd, J=7.0, 2.1 Hz, 1H), 8.56 (s, 1H), 8.51-8.43 (m, 1H), 8.29 (d, J=8.5 Hz, 1H), 8.18 (s, 1H), 7.90 (s, 1H), 7.79-7.72 (m, 2H), 7.49 (t, J=9.4 Hz, 1H), 3.98 (t, J=6.5 Hz, 2H), 3.77-3.35 (m, 8H), 1.59 (dd, J=13.5, 6.8 Hz, 2H), 0.89 (t, J=7.1 Hz, 3H).

LCMS (ESI-TOF) m/z 499.1 [M+H⁺] with a purity of >97%.

Propyl 4-(4-chloro-2-(4-fluoro-3-(methylcarbamoyl)phenyl)quinoline-7-carbonyl)piperazine-1-carboxylate (B152)

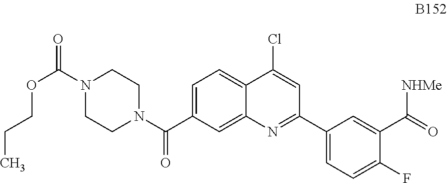 [SMILES: CCCOC(=O)N1CCN(C(=O)c2ccc3c(Cl)cc(-c4ccc(F)c(C(=O)NC)c4)nc3c2)CC1]

Compound B152 was synthesized according to General Procedure L using propyl 4-(2,4-dichloroquinoline-7-carbonyl)piperazine-1-carboxylate and 4-fluoro-3-(methylcarbamoyl)phenylboronic acid as starting materials.

¹H NMR (400 MHz, DMSO-d₆) δ 8.57 (s, 1H), 8.56 (s, 2H), 8.51-8.40 (m, 2H), 8.29 (d, J=8.5 Hz, 1H), 8.17 (s, 1H), 7.77 (d, J=8.3 Hz, 1H), 7.50 (t, J=9.2 Hz, 1H), 3.98 (t, J=6.5 Hz, 2H), 3.77-3.36 (m, 8H), 2.83 (d, J=4.5 Hz, 3H), 1.59 (dd, J=13.5, 6.2 Hz, 2H), 0.89 (t, J=7.0 Hz, 3H).

LCMS (ESI-TOF) m/z 513.1 [M+H⁺] with a purity of >97%.

### Propyl 4-(4-chloro-2-(thiazol-4-yl)quinoline-7-carbonyl)piperazine-1-carboxylate (B153)

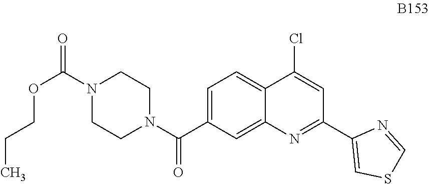 [SMILES: CCCOC(=O)N1CCN(C(=O)c2ccc3c(Cl)cc(-c4cscn4)nc3c2)CC1]

Compound B153 was synthesized according to General Procedure L using propyl 4-(2,4-dichloroquinoline-7-carbonyl)piperazine-1-carboxylate and thiazol-4-ylboronic acid pinacol ester as starting materials.

¹H NMR (400 MHz, DMSO-d₆) δ 9.33 (d, J=1.9 Hz, 1H), 8.63 (d, J=1.9 Hz, 1H), 8.48 (s, 1H), 8.29 (d, J=8.5 Hz, 1H), 8.12 (s, 1H), 7.77 (d, J=8.5 Hz, 1H), 3.98 (t, J=6.5 Hz, 2H), 3.80-3.35 (m, 8H), 1.59 (dd, J=14.1, 6.8 Hz, 2H), 0.89 (t, J=7.1 Hz, 3H).

LCMS (ESI-TOF) m/z 445.1 [M+H⁺] with a purity of >97%.

### Propyl 4-(4-chloro-2-(3-(dimethylcarbamoyl)-4-fluorophenyl)quinoline-7-carbonyl)piperazine-1-carboxylate (B154)

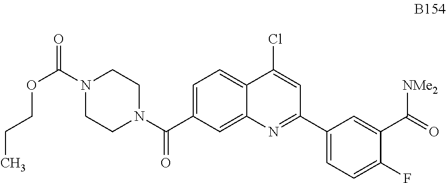 [SMILES: CCCOC(=O)N1CCN(C(=O)c2ccc3c(Cl)cc(-c4ccc(F)c(C(=O)N(C)C)c4)nc3c2)CC1]

Compound B154 was synthesized according to General Procedure L using propyl 4-(2,4-dichloroquinoline-7-carbonyl)piperazine-1-carboxylate and 3-(dimethylc arb amoyl) -4-fluorophenylboronic acid as starting materials.

¹H NMR (400 MHz, DMSO-d₆) δ 8.58 (s, 1H), 8.51-8.44 (m, 1H), 8.39 (d, J=6.2 Hz, 1H), 8.29 (d, J=8.6 Hz, 1H), 8.17 (s, 1H), 7.77 (d, J=8.5 Hz, 1H), 7.52 (t, J=9.0 Hz, 1H), 3.98 (t, J=6.5 Hz, 2H), 3.76-3.36 (m, 8H), 3.06 (s, 3H), 2.92 (s, 3H), 1.59 (dd, J=14.0, 7.3 Hz, 2H), 0.89 (t, J=7.0 Hz, 3H).

LCMS (ESI-TOF) m/z 527.2 [M+H⁺] with a purity of >97%.

### Propyl 4-(4-chloro-2-(1-methyl-1H-indazol-5-yl)quinoline-7-carbonyl)-2-methylpiperazine-1-carboxylate (B155)

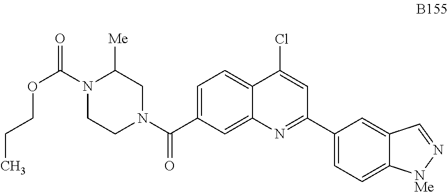 [SMILES: CCCOC(=O)N1CCN(C(=O)c2ccc3c(Cl)cc(-c4ccc5c(cnn5C)c4)nc3c2)CC1C]

Compound B155 was synthesized according to General Procedure L using propyl 4-(2,4-dichloroquinoline-7-carbonyl)-2-methylpiperazine-1-carboxylate and 1-methylindazol-5-ylboronic acid as starting materials.

¹H NMR (400 MHz, DMSO-d₆) δ 8.79 (s, 1H), 8.58 (s, 1H), 8.45 (d, J=8.8 Hz, 1H), 8.28 (d, J=8.8 Hz, 1H), 8.22 (s, 1H), 8.12 (br d, J=15.2 Hz, 1H), 7.82 (d, J=8.8 Hz, 1H), 7.72 (br s, 1H), 4.38 (br s, 2H), 4.11 (s, 3H), 4.01-3.46 (m, 5H), 3.22-2.99 (m, 2H), 1.63-1.54 (m, 2H), 1.13 (br d, J=68 Hz, 3H), 0.89 (t, J=7.6 Hz, 3H).

LCMS (ESI-TOF) m/z 506.3 [M+H⁺] with a purity of >95%.

### Propyl 4-(4-chloro-2-(3-hydroxy-4-methylphenyl)quinoline-7-carbonyl)piperazine-1-carboxylate (B156)

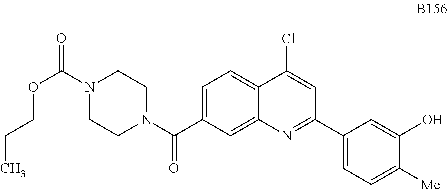 [SMILES: CCCOC(=O)N1CCN(C(=O)c2ccc3c(Cl)cc(-c4ccc(C)c(O)c4)nc3c2)CC1]

Compound B156 was synthesized according to General Procedure L using propyl 4-(2,4-dichloroquinoline-7-carbonyl)piperazine-1-carboxylate and 3-hydroxy-4-methylphenylboronic acid as starting materials.

¹H NMR (400 MHz, DMSO-d₆) δ 9.64 (s, 1H), 8.32 (s, 1H), 8.26 (d, J=8.5 Hz, 1H), 8.07 (s, 1H), 7.79 (s, 1H), 7.73 (d, J=8.5 Hz, 1H), 7.63 (d, J=7.9 Hz, 1H), 7.25 (d, J=7.8 Hz, 1H), 3.98 (t, J=6.6 Hz, 2H), 3.77-3.36 (m, 8H), 2.21 (s, 3H), 1.59 (dd, J=13.8, 7.2 Hz, 2H), 0.89 (t, J=7.2 Hz, 3H).

LCMS (ESI-TOF) m/z 468.1 [M+H⁺] with a purity of >99%.

### Propyl 4-(4-chloro-2-(4-fluoro-3-hydroxyphenyl)quinoline-7-carbonyl)piperazine-1-carboxylate (B157)

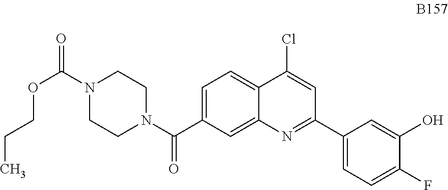 [SMILES: CCCOC(=O)N1CCN(C(=O)c2ccc3c(Cl)cc(-c4ccc(F)c(O)c4)nc3c2)CC1]

Compound B157 was synthesized according to General Procedure L using propyl 4-(2,4-dichloroquinoline-7-carbonyl)piperazine-1-carboxylate and 3-hydroxy-4-fluorophenylboronic acid as starting materials.

¹H NMR (400 MHz, DMSO-d₆) δ 10.24 (s, 1H), 8.39 (s, 1H), 8.27 (d, J=8.5 Hz, 1H), 8.09 (s, 1H), 7.96 (d, J=6.9 Hz, 1H), 7.74 (d, J=8.6 Hz, 2H), 7.32 (dd, J=10.8, 8.7 Hz, 1H), 3.98 (t, J=6.5 Hz, 2H), 3.74-3.36 (m, 8H), 1.59 (dd, J=13.8, 7.0 Hz, 2H), 0.89 (t, J=7.2 Hz, 3H).

LCMS (ESI-TOF) m/z 472.1 [M+H⁺] with a purity of >99%.

### Propyl 4-(4-chloro-2-(3-(1-hydroxyethyl)phenyl)quinoline-7-carbonyl)piperazine-1-carboxylate (B158)

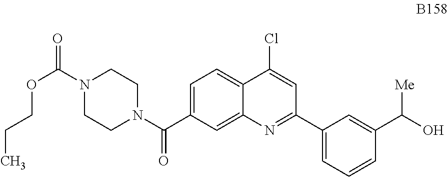 [SMILES: CCCOC(=O)N1CCN(C(=O)c2ccc3c(Cl)cc(-c4cccc(C(C)O)c4)nc3c2)CC1]

Compound B158 was synthesized according to General Procedure L using propyl 4-(2,4-dichloroquinoline-7-carbonyl)piperazine-1-carboxylate and 3-(1-hydroxyethyl)phenylboronic acid as starting materials.

¹H NMR (400 MHz, DMSO-d₆) δ 8.47 (s, 1H), 8.33-8.25 (m, 2H), 8.16 (s, 2H), 7.75 (d, J=8.5 Hz, 1H), 7.55-7.47 (m, 2H), 5.29 (d, J=4.1 Hz, 1H), 4.92-4.78 (m, 1H), 3.98 (t, J=6.5 Hz, 2H), 3.81-3.37 (m, 8H), 1.59 (dd, J=13.6, 6.7 Hz, 2H), 1.41 (d, J=6.4 Hz, 3H), 0.89 (t, J=7.2 Hz, 3H).

LCMS (ESI-TOF) m/z 482.2 [M+H⁺] with a purity of >99%.

### Propyl 4-(4-chloro-2-(3-(hydroxymethyl)-4-methylphenyl)quinoline-7-carbonyl)piperazine-1-carboxylate (B159)

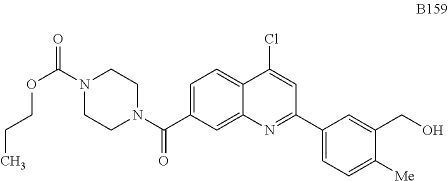 [SMILES: CCCOC(=O)N1CCN(C(=O)c2ccc3c(Cl)cc(-c4ccc(C)c(CO)c4)nc3c2)CC1]

Compound B159 was synthesized according to General Procedure L using propyl 4-(2,4-dichloroquinoline-7-carbonyl)piperazine-1-carboxylate and 3-(hydroxymethyl) -4-methylphenylboronic acid as starting materials.

¹H NMR (400 MHz, DMSO-d₆) δ 8.42 (s, 1H), 8.34 (s, 1H), 8.27 (d, J=8.5 Hz, 1H), 8.14 (s, 1H), 8.11 (d, J=7.5 Hz, 1H), 7.73 (d, J=8.7 Hz, 1H), 7.34 (d, J=7.9 Hz, 1H), 5.22 (t, J=5.4 Hz, 1H), 4.61 (d, J=5.3 Hz, 2H), 3.98 (t, J=6.6 Hz, 2H), 3.76-3.38 (m, 8H), 2.34 (s, 3H), 1.58 (dd, J=12.1, 5.4 Hz, 2H), 0.89 (t, J=7.2 Hz, 3H).

LCMS (ESI-TOF) m/z 482.1 [M+H⁺] with a purity of >98%.

### Propyl (S)-4-(4-chloro-2-(4-hydroxyphenyl)quinoline-7-carbonyl)-3-methylpiperazine-1-carboxylate (B160)

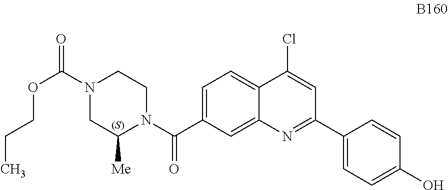

Compound B160 was synthesized according to General Procedure L using propyl (S)-4-(2,4-dichloroquinoline-7-carbonyl)-3-methylpiperazine-1-carboxylate and 4-hydroxyphenylboronic acid as starting materials.

¹H NMR (400 MHz, 80° C., DMSO-d₆) δ 8.26 (s, 1H), 8.22 (d, J=8.5 Hz, 1H), 8.15 (d, J=8.7 Hz, 2H), 8.01 (d, J=1.0 Hz, 1H), 7.63 (dd, J=8.5, 1.5 Hz, 1H), 6.93 (d, J=8.7 Hz, 2H), 4.48-3.70 (m, 6H), 3.33-2.90 (m, 3H), 1.65-1.53 (m, 2H), 1.20 (d, J=6.7 Hz, 3H), 0.90 (t, J=7.4 Hz, 3H).

LCMS (ESI-TOF) m/z 468.1 [M+H⁺] with a purity of >99%.

### Propyl (S)-4-(4-chloro-2-(4-(fluoromethyl)phenyl)quinoline-7-carbonyl)-3-methylpiperazine-1-carboxylate (B161)

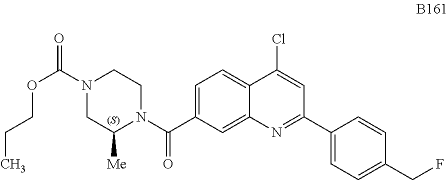

Compound B161 was synthesized according to General Procedure L using propyl (S)-4-(2,4-dichloroquinoline-7-carbonyl)-3-methylpiperazine-1-carboxylate and 4-(fluoromethyl)phenylboronic acid pinacol ester as starting materials.

¹H NMR (400 MHz, 80° C., DMSO-d₆) δ 8.40 (s, 1H), 8.34 (d, J=7.7 Hz, 2H), 8.28 (d, J=8.5 Hz, 1H), 8.10 (d, J=1.1 Hz, 1H), 7.71 (dd, J=8.5, 1.5 Hz, 1H), 7.60 (d, J=6.9 Hz, 2H), 5.52 (d, J=47.5 Hz, 2H), 4.43-3.74 (m, 6H), 3.30-2.94 (m, 3H), 1.66-1.52 (m, 2H), 1.21 (d, J=6.7 Hz, 3H), 0.90 (t, J=7.4 Hz, 3H).

LCMS (ESI-TOF) m/z 484.1 [M+H⁺] with a purity of >95%.

### Propyl (S)-4-(4-chloro-2-(3-fluoro-4-hydroxyphenyl)quinoline-7-carbonyl)-3-methylpiperazine-1-carboxylate (B162)

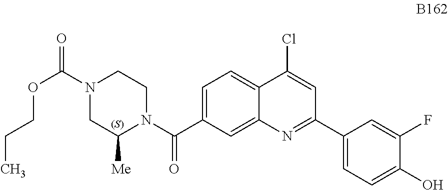

Compound B162 was synthesized according to General Procedure L using propyl (S)-4-(2,4-dichloroquinoline-7-carbonyl)-3-methylpiperazine-1-carboxylate and 3-fluoro-4-hydroxyphenylboronic acid as starting materials.

¹H NMR (400 MHz, 80° C., DMSO-d₆) δ 8.32 (s, 1H), 8.23 (d, J=8.5 Hz, 1H), 8.09 (dd, J=12.9, 2.1 Hz, 1H), 8.04 (d, J=1.1 Hz, 1H), 7.98 (dd, J=8.5, 1.4 Hz, 1H), 7.66 (dd, J=8.5, 1.5 Hz, 1H), 7.10 (t, J=8.8 Hz, 1H), 4.49-3.66 (m, 6H), 3.30-2.87 (m, 3H), 1.68-1.52 (m, 2H), 1.20 (d, J=6.7 Hz, 3H), 0.90 (t, J=7.4 Hz, 3H).

LCMS (ESI-TOF) m/z 486.1 [M+H⁺] with a purity of >96%.

### Propyl 4-(2-(benzo[d][1,3]dioxol-5-yl)-4-chloroquinoline-7-carbonyl)piperazine-1-carboxylate (B163)

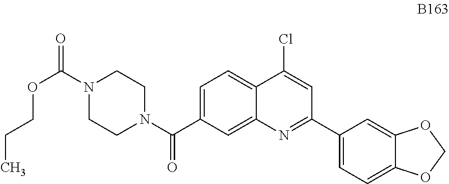 [SMILES: CCCOC(=O)N1CCN(C(=O)c2ccc3c(Cl)cc(-c4ccc5c(c4)OCO5)nc3c2)CC1]

Compound B163 was synthesized according to General Procedure L using propyl 4-(2,4-dichloroquinoline-7-carbonyl)piperazine-1-carboxylate and 3,4-(methylenedioxy)phenylboronic acid as starting materials.

¹H NMR (400 MHz, 80° C., DMSO-d₆) δ 8.32 (s, 1H), 8.23 (d, J=8.6 Hz, 1H), 8.08 (d, J=1.4 Hz, 1H), 7.88 (d, J=1.9 Hz, 1H), 7.86 (s, 1H), 7.69 (dd, J=8.6, 1.4 Hz, 1H), 7.06 (d, J=8.8 Hz, 1H), 6.11 (s, 2H), 3.99 (t, J=6.6 Hz, 2H), 3.63-3.38 (m, 8H), 1.69-1.53 (m, 2H), 0.90 (t, J=7.4 Hz, 3H).

LCMS (ESI-TOF) m/z 482.1 [M+H⁺] with a purity of >99%.

### Propyl 4-(2-(1H-benzo[d][1,2,3]triazol-6-yl)-4-chloroquinoline-7-carbonyl)piperazine-1-carboxylate (B164)

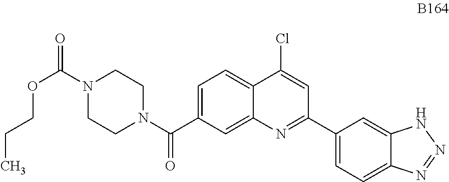 [SMILES: CCCOC(=O)N1CCN(C(=O)c2ccc3c(Cl)cc(-c4ccc5nn[nH]c5c4)nc3c2)CC1]

Compound B164 was synthesized according to General Procedure L using propyl 4-(2,4-dichloroquinoline-7-carbonyl)piperazine-1-carboxylate and 1H-1,2,3-benzotriazol-5-ylboronic acid as starting materials.

¹H NMR (400 MHz, DMSO-d₆) δ 8.88 (s, 1H), 8.67 (s, 1H), 8.45 (d, J=8.3 Hz, 1H), 8.30 (d, J=8.5 Hz, 1H), 8.18 (s, 1H), 8.02 (d, J=9.0 Hz, 1H), 7.76 (d, J=8.7 Hz, 1H), 3.98 (t, J=6.5 Hz, 2H), 3.79-3.37 (m, 8H), 1.59 (dd, J=13.7, 7.2 Hz, 2H), 0.89 (t, J=7.2 Hz, 3H).

LCMS (ESI-TOF) m/z 479.1 [M+H⁺] with a purity of >98%.

### Propyl 4-(4-chloro-2-(3-fluoro-4-methoxyphenyl)quinoline-7-carbonyl)piperazine-1-carboxylate (B165)

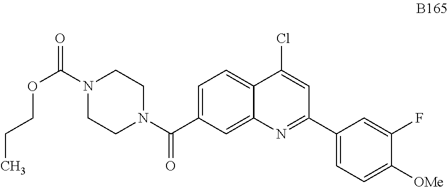 [SMILES: CCCOC(=O)N1CCN(C(=O)c2ccc3c(Cl)cc(-c4ccc(OC)c(F)c4)nc3c2)CC1]

Compound B165 was synthesized according to General Procedure L using propyl 4-(2,4-dichloroquinoline-7-carbonyl)piperazine-1-carboxylate and 3-fluoro-4-methoxyphenylboronic acid as starting materials.

¹H NMR (400 MHz, DMSO-d₆) δ 8.50 (s, 1H), 8.26 (d, J=8.6 Hz, 1H), 8.24-8.17 (m, 2H), 8.12 (s, 1H), 7.73 (dd, J=8.6, 1.3 Hz, 1H), 7.36 (t, J=8.7 Hz, 1H), 3.98 (t, J=6.6 Hz, 2H), 3.95 (s, 3H), 3.78-3.35 (m, 8H), 1.59 (dd, J=13.8, 6.8 Hz, 2H), 0.89 (t, J=7.2 Hz, 3H).

LCMS (ESI-TOF) m/z 486.1 [M+H⁺] with a purity of >99%.

### Propyl 4-(4-chloro-2-(2-methylthiazol-5-yl)quinoline-7-carbonyl)piperazine-1-carboxylate (B166)

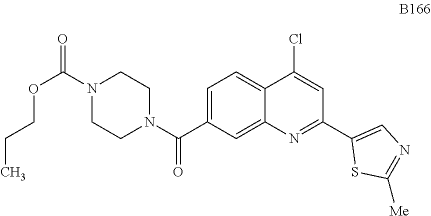 [SMILES: CCCOC(=O)N1CCN(C(=O)c2ccc3c(Cl)cc(-c4cnc(C)s4)nc3c2)CC1]

Compound B166 was synthesized according to General Procedure L using propyl 4-(2,4-dichloroquinoline-7-carbonyl)piperazine-1-carboxylate and 2-methylthiazol-5-yl-boronic acid pinacol ester as starting materials.

¹H NMR (400 MHz, DMSO-d₆) δ 8.65 (s, 1H), 8.53 (s, 1H), 8.25 (d, J=8.5 Hz, 1H), 8.01 (s, 1H), 7.72 (dd, J=8.5, 1.1 Hz, 1H), 3.98 (t, J=6.6 Hz, 2H), 3.77-3.36 (m, 8H), 1.59 (dd, J=13.8, 6.9 Hz, 2H), 0.89 (t, J=7.3 Hz, 3H).

LCMS (ESI-TOF) m/z 459.1 [M+H⁺] with a purity of >98%.

### Propyl 4-(4-chloro-2-(3-((dimethylamino)methyl)-4-fluorophenypquinoline-7-carbonyl)piperazine-1-carboxylate (B167)

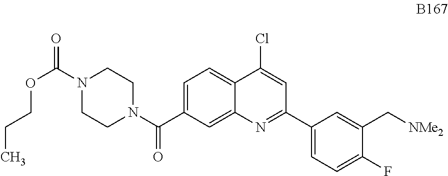 [SMILES: CCCOC(=O)N1CCN(C(=O)c2ccc3c(Cl)cc(-c4ccc(F)c(CN(C)C)c4)nc3c2)CC1]

Compound B167 was synthesized according to General Procedure L using propyl 4-(2,4-dichloroquinoline-7-carbonyl)piperazine-1-carboxylate and 3-((dimethylamino)methyl)-4-fluorophenylboronic acid as starting materials.

¹H NMR (400 MHz, DMSO-d₆) δ 8.49 (s, 1H), 8.38 (dd, J=7.2, 2.1 Hz, 1H), 8.31-8.24 (m, 2H), 8.15 (s, 1H), 7.75 (dd, J=8.5, 1.2 Hz, 1H), 7.37 (t, J=9.2 Hz, 1H), 3.98 (t, J=6.6 Hz, 2H), 3.79-3.37 (m, 10H), 2.22 (s, 6H), 1.59 (dd, J=13.8, 6.8 Hz, 2H), 0.89 (t, J=7.2 Hz, 3H).

LCMS (ESI-TOF) m/z 513.1 [M+AH⁺] with a purity of >97%.

### Propyl 4-(4-chloro-2-(1-methyl-1H-pyrazol-3-yl)quinoline-7-carbonyl)piperazine-1-carboxylate (B168)

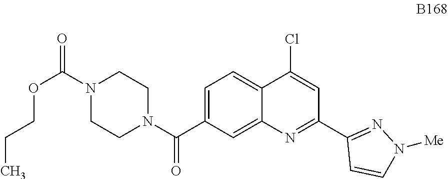 [SMILES: CCCOC(=O)N1CCN(C(=O)c2ccc3c(Cl)cc(-c4ccn(C)n4)nc3c2)CC1]

Compound B168 was synthesized according to General Procedure L using propyl 4-(2,4-dichloroquinoline-7-carbonyl)piperazine-1-carboxylate and (1-methyl-1H-yrazol-3-yl)1boronic acid as starting materials.

¹H NMR (400 MHz, DMSO-d₆) δ 8.26 (s, 1H), 8.25 (d, J=8.9 Hz, 1H), 8.07 (s, 1H), 7.88 (d, J=2.0 Hz, 1H), 7.72 (d, J=8.5 Hz, 1H), 7.01 (d, J=2.1 Hz, 1H), 4.02-3.96 (m, 5H), 3.77-3.36 (m, 8H), 1.59 (dd, J=13.6, 6.9 Hz, 2H), 0.89 (t, J=7.2 Hz, 3H).

LCMS (ESI-TOF) m/z 442.1 [M+H⁺] with a purity of >96%.

Propyl (S)-4-(4-chloro-2-(1H-indazol-5-yl)quinoline-7-carbonyl)-2-methylpiperazine-1-carboxylate (B169)

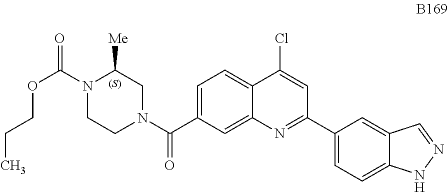

Compound B169 was synthesized according to General Procedure L using propyl (S)-4-(2,4-dichloroquinoline-7-carbonyl)-2-methylpiperazine-1-carboxylate and 1H-indazolyl-5-boronic acid as starting materials.

¹H NMR (400 MHz, 80° C., DMSO-d₆) δ 13.12 (s, 1H), 8.76 (s, 1H), 8.48 (s, 1H), 8.39 (d, J=9.5 Hz, 1H), 8.29 (d, J=8.6 Hz, 1H), 8.23 (s, 1H), 8.12 (s, 1H), 7.78-7.65 (m, 2H), 4.42-3.61 (m, 6H), 3.43-3.11 (m, 3H), 1.62 (dd, J=13.8, 6.9 Hz, 2H), 1.17 (d, J=5.5 Hz, 3H), 0.92 (t, J=7.4 Hz, 3H).

LCMS (ESI-TOF) m/z 492.1 [M+H⁺] with a purity of >95%.

### Propyl (S)-4-(4-chloro-2-(2-methyl-2H-indazol-5-yl)quinoline-7-carbonyl)-2-methylpiperazine-1-carboxylate (B170)

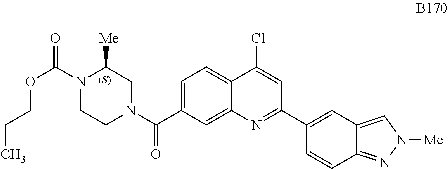

Compound B170 was synthesized according to General Procedure L using propyl (S)-4-(2,4-dichloroquinoline-7-carbonyl)-2-methylpiperazine-1-carboxylate and 2-methylindazolyl-5-boronic acid pinacol ester as starting materials.

¹H NMR (400 MHz, 80° C., DMSO-d₆) δ 8.70 (s, 1H), 8.49 (s, 1H), 8.46 (s, 1H), 8.31-8.22 (m, 2H), 8.12 (s, 1H), 7.81-7.61 (m, 2H), 4.43-3.77 (m, 9H), 3.42-3.13 (m, 3H), 1.71-1.54 (m, 2H), 1.17 (d, J=5.7 Hz, 3H), 0.92 (t, J=7.4 Hz, 3H).

LCMS (ESI-TOF) m/z 506.1 [M+H⁺] with a purity of >95%.

### Propyl (S)-4-(4-chloro-2-(3-(hydroxymethyl)-4-methoxyphenyl)quinoline-7-carbonyl)-2-methylpiperazine-1-carboxylate (B171)

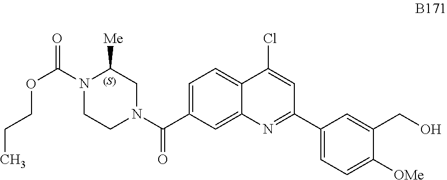

Compound B171 was synthesized according to General Procedure L using propyl (S)-4-(2,4-dichloroquinoline-7-carbonyl)-2-methylpiperazine-1-carboxylate and 3-hydroxymethyl-4-methylphenylboronic acid as starting materials.

¹H NMR (400 MHz, 80° C., DMSO-d₆) δ 8.35 (s, 1H), 8.29 (s, 1H), 8.25 (d, J=8.5 Hz, 1H), 8.17 (dd, J=8.5, 2.3 Hz, 1H), 8.07 (s, 1H), 7.68 (d, J=8.5 Hz, 1H), 7.12 (d, J=8.6 Hz, 1H), 4.87 (s, 1H), 4.60 (d, J=4.7 Hz, 2H), 4.31-3.66 (m, 9H), 3.44-3.10 (m, 3H), 1.64-1.52 (m, 2H), 1.13 (d, J=6.0 Hz, 3H), 0.89 (t, J=7.4 Hz, 3H).

LCMS (ESI-TOF) m/z 512.2 [M+H⁺] with a purity of >96%.

### Propyl 4-(2-(3-(aminomethyl)-4-fluorophenyl)-4-chloroquinoline-7-carbonyl)piperazine-1-carboxylate (B172)

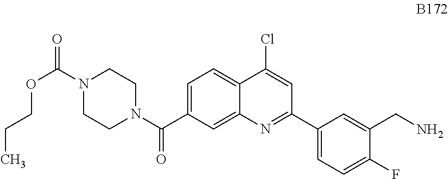 [SMILES: CCCOC(=O)N1CCN(C(=O)c2ccc3c(Cl)cc(-c4ccc(F)c(CN)c4)nc3c2)CC1]

Compound B172 was synthesized according to General Procedure L using propyl 4-(2,4-dichloroquinoline-7-carbonyl)piperazine-1-carboxylate and 3-(aminomethyl)-4-fluorophenylboronic acid pinacol ester as starting materials.

¹H NMR (400 MHz, DMSO-d₆) δ 8.50 (s, 1H), 8.47 (d, J=7.1 Hz, 1H), 8.28 (d, J=8.6 Hz, 1H), 8.26-8.21 (m, 1H), 8.14 (s, 1H), 7.75 (d, J=8.5 Hz, 1H), 7.33 (t, J=9.1 Hz, 1H), 3.98 (t, J=6.5 Hz, 2H), 3.86 (s, 2H), 3.77-3.38 (m, 8H), 1.59 (dd, J=14.0, 7.1 Hz, 2H), 0.89 (t, J=6.6 Hz, 3H).

LCMS (ESI-TOF) m/z 485.1 [M+H⁺] with a purity of >95%.

### Propyl 4-(4-chloro-2-(3-methyl-1H-pyrazolo[3,4-b]pyridin-5-yl)quinoline-7-carbonyl)piperazine-1-carboxylate (B173)

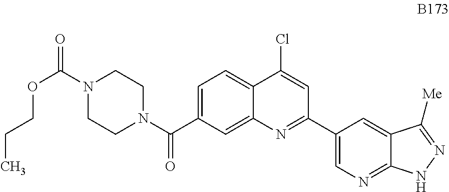 [SMILES: CCCOC(=O)N1CCN(C(=O)c2ccc3c(Cl)cc(-c4cnc5[nH]nc(C)c5c4)nc3c2)CC1]

Compound B173 was synthesized according to General Procedure L using propyl 4-(2,4-dichloroquinoline-7-carbonyl)piperazine-1-carboxylate and 3-methyl-1H-pyrazolo [3 ,4-11] pyridine-5-boronic acid pinacol ester as starting materials.

¹H NMR (400 MHz, DMSO-d₆) δ 13.46 (s, 1H), 9.49 (d, J=1.8 Hz, 1H), 9.15 (s, 1H), 8.67 (s, 1H), 8.29 (d, J=8.5 Hz, 1H), 8.18 (s, 1H), 7.75 (d, J=8.4 Hz, 1H), 3.98 (t, J=6.6 Hz, 2H), 3.83-3.40 (m, 8H), 2.61 (s, 3H), 1.59 (dd, J=13.6, 6.7 Hz, 2H), 0.89 (t, J=7.2 Hz, 3H).

LCMS (ESI-TOF) m/z 493.1 [M+H⁺] with a purity of >99%.

### Propyl 4-(4-chloro-2-(1-methyl-1H-pyrazolo[3,4-b]pyridin-5-yl)quinoline-7-carbonyl)piperazine-1-carboxylate (B174)

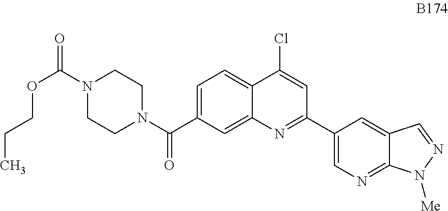 [SMILES: CCCOC(=O)N1CCN(C(=O)c2ccc3c(Cl)cc(-c4cnc5c(cnn5C)c4)nc3c2)CC1]

Compound B174 was synthesized according to General Procedure L using propyl 4-(2,4-dichloroquinoline-7-carbonyl)piperazine-1-carboxylate and (1-methyl-1H-pyrazolo [3 ,4-b]pyridin-5-yl)boronic acid pinacol ester as starting materials.

¹H NMR (400 MHz, DMSO-d₆) δ 9.54 (s, 1H), 9.17 (s, 1H), 8.65 (s, 1H), 8.33 (s, 1H), 8.30 (d, J=8.5 Hz, 1H), 8.18 (s, 1H), 7.76 (d, J=8.5 Hz, 1H), 4.14 (s, 3H), 3.98 (t, J=6.6 Hz, 2H), 3.78-3.37 (m, 8H), 1.59 (dd, J=12.9, 5.3 Hz, 2H), 0.89 (t, J=7.2 Hz, 3H).

LCMS (ESI-TOF) m/z 493.1 [M+H⁺] with a purity of >95%.

### Propyl 4-(4-chloro-2-(2-methyl-1H-pyrrolo[2,3-b]pyridin-5-yl)quinoline-7-carbonyl)piperazine-1-carboxylate (B175)

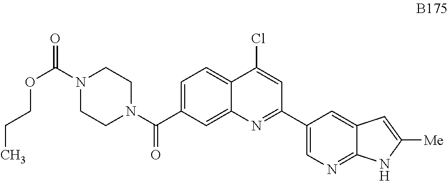 [SMILES: CCCOC(=O)N1CCN(C(=O)c2ccc3c(Cl)cc(-c4cnc5[nH]c(C)cc5c4)nc3c2)CC1]

Compound B175 was synthesized according to General Procedure L using propyl 4-(2,4-dichloroquinoline-7-carbonyl)piperazine-1-carboxylate and 2-methyl-1H-pyrrolo [2,3-b]pyridine-5-boronic acid pinacol ester as starting materials.

¹H NMR (400 MHz, DMSO-d₆) δ 11.73 (s, 1H), 9.09 (s, 1H), 8.73 (s, 1H), 8.55 (s, 1H), 8.26 (d, J=8.6 Hz, 1H), 8.13 (s, 1H), 7.72 (d, J=8.5 Hz, 1H), 6.30 (s, 1H), 3.98 (t, J=6.5 Hz, 2H), 3.80-3.39 (m, 8H), 2.44 (s, 3H), 1.68-1.51 (m, 2H), 0.90 (t, J=5.9 Hz, 3H).

LCMS (ESI-TOF) m/z 492.1 [M+H⁺] with a purity of >96%.

### Propyl 4-(4-chloro-2-(3-methyl-1H-indazol-5-yl)quinoline-7-carbonyl)piperazine-1-carboxylate (B176)

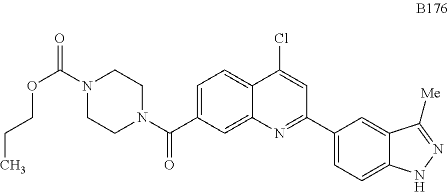 [SMILES: CCCOC(=O)N1CCN(C(=O)c2ccc3c(Cl)cc(-c4ccc5[nH]nc(C)c5c4)nc3c2)CC1]

Compound B176 was synthesized according to General Procedure L using propyl 4-(2,4-dichloroquinoline-7-carbonyl)piperazine-1-carboxylate and 3-methyl-1H-indazole-5-boronic acid as starting materials.

¹H NMR (400 MHz, DMSO-d₆) δ 13.46 (s, 1H), 9.49 (d, J=1.8 Hz, 1H), 9.15 (s, 1H), 8.67 (s, 1H), 8.29 (d, J=8.5 Hz, 1H), 8.18 (s, 1H), 7.75 (d, J=8.4 Hz, 1H), 3.98 (t, J=6.6 Hz, 2H), 3.84-3.39 (m, 8H), 2.61 (s, 3H), 1.59 (dd, J=13.6, 6.7 Hz, 2H), 0.89 (t, J=7.2 Hz, 3H).

LCMS (ESI-TOF) m/z 493.1 [M+H⁺] with a purity of >99%.

### Propyl (R)-4-(4-chloro-2-(6-methylpyridin-3-yl)quinoline-7-carbonyl)-2-methylpiperazine-1-carboxylate (B177)

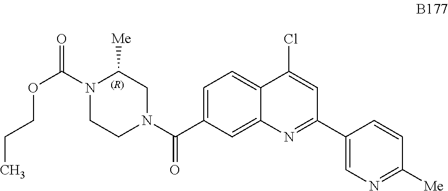

Compound B177 was synthesized according to General Procedure L using propyl (R)-4-(2,4-dichloroquinoline-7-carbonyl)-2-methylpiperazine-1-carboxylate and 2-picoline-5-boronic acid as starting materials.

¹H NMR (400 MHz, 80° C., DMSO-d₆) δ 9.33 (d, J=2.1 Hz, 1H), 8.52 (dd, J=8.1, 2.2 Hz, 1H), 8.45 (s, 1H), 8.29 (d, J=8.6 Hz, 1H), 8.12 (s, 1H), 7.74 (d, J=8.4 Hz, 1H), 7.43 (d, J=8.2 Hz, 1H), 4.50-3.69 (m, 6H), 3.39-3.09 (m, 3H), 2.57 (s, 3H), 1.72-1.47 (m, 2H), 1.14 (d, J=6.0 Hz, 3H), 0.89 (t, J=7.4 Hz, 3H).

LCMS (ESI-TOF) m/z 467.1 [M+H⁺] with a purity of >95%.

### Propyl (R)-4-(4-chloro-2-(6-(methoxycarbonyl)pyridin-3-yl)quinoline-7-carbonyl)-2-methylpiperazine-1-carboxylate (B178)

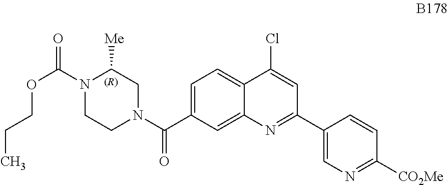

Compound B178 was synthesized according to General Procedure L using propyl (R)-4-(2,4-dichloroquinoline-7-carbonyl)-2-methylpiperazine-1-carboxylate and 6-(methoxycarbonyl)pyridine-5-boronic acid pinacol ester as starting materials.

¹H NMR (400 MHz, 80° C., DMSO-d₆) δ 9.57 (d, J=1.8 Hz, 1H), 8.82 (dd, J=8.2, 2.3 Hz, 1H), 8.57 (s, 1H), 8.33 (d, J=8.5 Hz, 1H), 8.23-8.19 (m, 1H), 8.18 (s, 1H), 7.79 (d, J=8.6 Hz, 1H), 4.39-3.53 (m, 9H), 3.18 (dd, J=54.4, 40.9 Hz, 3H), 1.59 (dt, J=14.1, 7.0 Hz, 2H), 1.14 (d, J=6.1 Hz, 3H), 0.89 (t, J=7.4 Hz, 3H).

LCMS (ESI-TOF) m/z 511.1 [M+H⁺] with a purity of >94%.

### Propyl 4-(4-chloro-2-(1-methyl-1H-pyrazol-4-yl)quinoline-7-carbonyl)piperazine-1-carboxylate (B179)

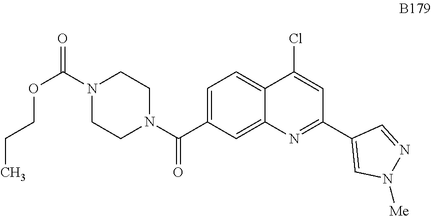 [SMILES: CCCOC(=O)N1CCN(C(=O)c2ccc3c(Cl)cc(-c4cnn(C)c4)nc3c2)CC1]

Compound B179 was synthesized according to General Procedure L using propyl 4-(2,4-dichloroquinoline-7-carbonyl)piperazine-1-carboxylate and 1-methyl-1H-pyrazol-4-ylboronic acid as starting materials.

¹H NMR (400 MHz, DMSO-d₆) δ 8.54 (s, 1H), 8.25-8.20 (m, 2H), 8.20 (d, J=8.2 Hz, 1H), 7.97 (s, 1H), 7.65 (d, J=8.6 Hz, 1H), 3.98 (t, J=6.6 Hz, 2H), 3.93 (s, 3H), 3.77-3.35 (m, 8H), 1.58 (dd, J=13.4, 6.1 Hz, 2H), 0.89 (t, J=7.1 Hz, 3H).

LCMS (ESI-TOF) m/z 442.1 [M+H⁺] with a purity of >95%.

### Propyl 4-(2-(3-amino-4-fluorophenyl)-4-chloroquinoline-7-carbonyl)piperazine-1-carboxylate (B180)

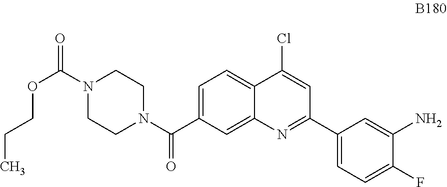 [SMILES: CCCOC(=O)N1CCN(C(=O)c2ccc3c(Cl)cc(-c4ccc(F)c(N)c4)nc3c2)CC1]

Compound B180 was synthesized according to General Procedure L using propyl 4-(2,4-dichloroquinoline-7-carbonyl)piperazine-1-carboxylate and 3-amino-4-fluorophenylboronic acid as starting materials.

¹H NMR (400 MHz, 80° C., DMSO-d₆) δ 8.37 (s, 1H), 8.26 (t, J=8.4 Hz, 3H), 8.12 (d, J=1.1 Hz, 1H), 7.72 (dd, J=8.5, 1.5 Hz, 1H), 7.48 (d, J=8.2 Hz, 2H), 3.99 (t, J=6.6 Hz, 2H), 3.63-3.41 (m, 8H), 1.65-1.52 (m, 2H), 0.90 (t, J=7.4 Hz, 3H).

LCMS (ESI-TOF) m/z 495.1 [M+H⁺] with a purity of >99%.

### Propyl 4-(4-chloro-2-(3-ethoxy-4-hydroxyphenyl)quinoline-7-carbonyl)piperazine-1-carboxylate (B181)

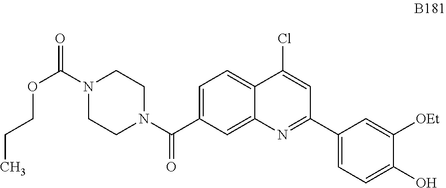 [SMILES: CCCOC(=O)N1CCN(C(=O)c2ccc3c(Cl)cc(-c4ccc(O)c(OCC)c4)nc3c2)CC1]

Compound B181 was synthesized according to General Procedure L using propyl 4-(2,4-dichloroquinoline-7-carbonyl)piperazine-1-carboxylate and 3-ethoxy-4-hydroxyphenylboronic acid pinacol ester as starting materials.

¹H NMR (400 MHz, 80° C., DMSO-d₆) δ 8.30 (s, 1H), 8.21 (d, J=8.5 Hz, 1H), 8.06 (s, 1H), 7.87 (d, J=1.9 Hz, 1H), 7.77 (dd, J=8.3, 2.0 Hz, 1H), 7.65 (dd, J=8.5, 1.3 Hz, 1H), 6.94 (d, J=8.3 Hz, 1H), 4.20 (q, J=6.9 Hz, 2H), 3.99 (t, J=6.6 Hz, 2H), 3.75-3.40 (m, 8H), 1.68-1.51 (m, 2H), 1.39 (t, J=7.0 Hz, 3H), 0.90 (t, J=7.4 Hz, 3H).

LCMS (ESI-TOF) m/z 498.1 [M+H⁺] with a purity of >99%.

### Propyl (S)-4-(4-chloro-2-(4-hydroxy-3-(hydroxymethyl)phenyl)quinoline-7-carbonyl)-2-methylpiperazine-1-carboxylate (B182)

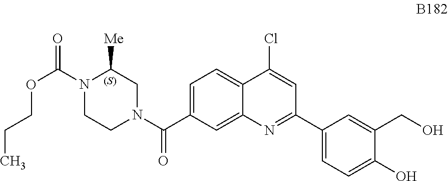

Compound B182 was synthesized according to General Procedure L using propyl (S)-4-(2,4-dichloroquinoline-7-carbonyl)-2-methylpiperazine-1-carboxylate and 3-(hydroxymethyl)-4-hydroxyphenylboronic acid as starting materials.

¹H NMR (400 MHz, 80° C., DMSO-d₆) 6 8.2-8.18 (m, 3H), 8.07-7.98 (m, 2H), 7.65 (d, J=8.5 Hz, 1H), 6.93 (d, J=8.4 Hz, 1H), 4.61 (s, 2H), 4.33-3.64 (m, 6H), 3.36-3.15 (m, 3H), 1.68-1.52 (m, 2H), 1.13 (d, J=6.5 Hz, 3H), 0.89 (t, J=7.4 Hz, 3H).

LCMS (ESI-TOF) m/z 498.1 [H+H⁺] with a purity of >97%.

### Propyl 4-(4-chloro-2-(1-methyl-1H-benzo[d]imidazol-5-yl)quinoline-7-carbonyl)piperazine-1-carboxylate (B183)

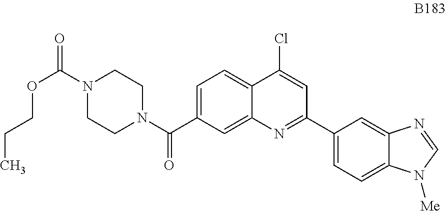 [SMILES: CCCOC(=O)N1CCN(C(=O)c2ccc3c(Cl)cc(-c4ccc5c(c4)ncn5C)nc3c2)CC1]

Compound B183 was synthesized according to General Procedure L using propyl 4-(2,4-dichloroquinoline-7-carbonyl)piperazine-1-carboxylate and (1-methyl-1H-benzimidazol-5-yl)boronic acid as starting materials.

¹H NMR (400 MHz, DMSO-d₆) δ 8.66 (d, J=0.8 Hz, 1H), 8.59 (s, 1H), 8.35 (dd, J=8.7, 1.0 Hz, 1H), 8.30 (s, 1H), 8.27 (d, J=8.5 Hz, 1H), 8.15 (s, 1H), 7.75 (d, J=8.6 Hz, 1H), 7.72 (dd, J=8.9, 1.1 Hz, 1H), 3.98 (t, J=6.6 Hz, 2H), 3.91 (s, 3H), 3.79-3.35 (m, 8H), 1.64-1.53 (m, 2H), 0.89 (t, J=7.4 Hz, 3H).

LCMS (ESI-TOF) m/z 492.1 [M+H⁺] with a purity of >98%.

### Propyl 4-(4-chloro-2-(2-hydroxy-1H-benzo[d]imidazol-5-yl)quinoline-7-carbonyl)piperazine-1-carboxylate (B184)

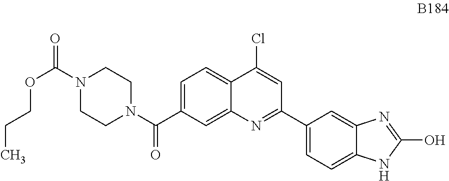 [SMILES: CCCOC(=O)N1CCN(C(=O)c2ccc3c(Cl)cc(-c4ccc5[nH]c(O)nc5c4)nc3c2)CC1]

Compound B184 was synthesized according to General Procedure L using propyl 4-(2,4-dichloroquinoline-7-carbonyl)piperazine-1-carboxylate and 2-hydroxybenzimidazole-5-boronic acid pinacol ester as starting materials.

¹H NMR (400 MHz, DMSO-d₆) δ 10.90 (d, J=4.9 Hz, 2H), 8.42 (s, 1H), 8.24 (d, J=8.5 Hz, 1H), 8.09 (s, 1H), 7.96 (d, J=8.6 Hz, 1H), 7.94 (s, 1H), 7.70 (d, J=8.5 Hz, 1H), 7.08 (d, J=8.1 Hz, 1H), 3.98 (t, J=6.5 Hz, 2H), 3.72-3.36 (m, 8H), 1.59 (dd, J=13.7, 6.9 Hz, 2H), 0.89 (t, J=7.3 Hz, 3H).

LCMS (ESI-TOF) m/z 494.1 [M+H⁺] with a purity of >96%.

### Propyl 4-(2-(2-aminopyrimidin-5-yl)-4-chloroquinoline-7-carbonyl)piperazine-1-carboxylate (B185)

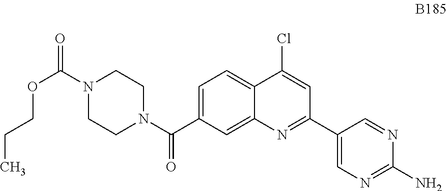 [SMILES: CCCOC(=O)N1CCN(C(=O)c2ccc3c(Cl)cc(-c4cnc(N)nc4)nc3c2)CC1]

Compound B185 was synthesized according to General Procedure L using propyl 4-(2,4-dichloroquinoline-7-carbonyl)piperazine-1-carboxylate and 2-aminopyrimidine-5-boronic acid as starting materials.

¹H NMR (400 MHz, DMSO-d₆) δ 9.17 (s, 2H), 8.43 (s, 1H), 8.23 (d, J=8.5 Hz, 1H), 8.07 (s, 1H), 7.69 (d, J=8.3 Hz, 1H), 7.23 (s, 2H), 3.98 (t, J=6.6 Hz, 2H), 3.73-3.35 (m, 8H), 1.59 (dd, J=14.0, 6.8 Hz, 2H), 0.89 (t, J=7.1 Hz, 3H).

LCMS (ESI-TOF) m/z 455.1 [M+H⁺] with a purity of >98%.

### Propyl 4-(4-chloro-2-(7-methyl-1H-indazol-5-yl)quinoline-7-carbonyl)piperazine-1-carboxylate (B186)

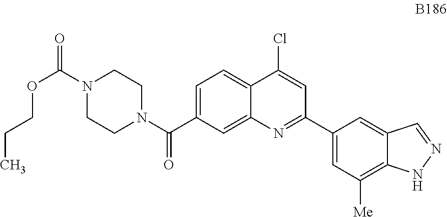 [SMILES: CCCOC(=O)N1CCN(C(=O)c2ccc3c(Cl)cc(-c4cc(C)c5[nH]ncc5c4)nc3c2)CC1]

Compound B186 was synthesized according to General Procedure L using propyl 4-(2,4-dichloroquinoline-7-carbonyl)piperazine-1-carboxylate and (7-methyl-1H-indazol-5-yl)boronic acid as starting materials.

¹H NMR (400 MHz, DMSO-d₆) δ 13.36 (s, 1H), 8.60 (s, 1H), 8.53 (s, 1H), 8.26 (d, J=8.5 Hz, 1H), 8.22 (s, 1H), 8.19 (s, 1H), 8.13 (d, J=1.0 Hz, 1H), 7.71 (dd, J=8.5, 1.5 Hz, 1H), 3.98 (t, J=6.6 Hz, 2H), 3.82-3.34 (m, 8H), 2.64 (s, 3H), 1.59 (dd, J=14.1, 7.1 Hz, 2H), 0.89 (t, J=7.2 Hz, 3H).

LCMS (ESI-TOF) m/z 492.1 [M+H⁺] with a purity of >97%.

### Propyl 4-(2-(1H-benzo[d]imidazol-4-yl)-4-chloroquinoline-7-carbonyl)piperazine-1-carboxylate (B187)

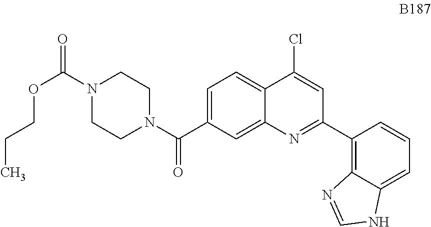 [SMILES: CCCOC(=O)N1CCN(C(=O)c2ccc3c(Cl)cc(-c4cccc5[nH]cnc45)nc3c2)CC1]

Compound B187 was synthesized according to General Procedure L using propyl 4-(2,4-dichloroquinoline-7-carbonyl)piperazine-1-carboxylate and 1H-benzimidazol-4-ylboronic acid as starting materials.

¹H NMR (400 MHz, DMSO-d₆) δ 12.85 (s, 1H), 8.69 (br s, 1H), 8.39 (s, 1H), 8.29 (d, J=8.4 Hz, 2H), 7.84 (s, 1H), 7.77 (d, J=8.4 Hz, 1H), 7.40 (t, J=7.7 Hz, 1H), 3.99 (t, J=6.6 Hz, 2H), 3.80-3.37 (m, 8H), 1.59 (dd, J=13.8, 7.6 Hz, 2H), 0.89 (t, J=7.4 Hz, 3H).

LCMS (ESI-TOF) m/z 478.1 [M+H⁺] with a purity of >98%.

### Propyl (S)-4-(4-chloro-2-(1,5-naphthyridin-3-yl)quinoline-7-carbonyl)-2-methylpiperazine-1-carboxylate (B188)

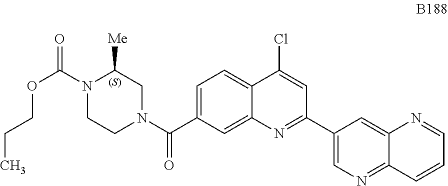

Compound B188 was synthesized according to General Procedure L using propyl (S)-4-(2,4-dichloroquinoline-7-carbonyl)-2-methylpiperazine-1-carboxylate and 1,5-naphthyridin-2-ylboronic acid pinacol ester as starting materials.

¹H NMR (400 MHz, 80° C., DMSO-d₆) δ 9.91 (d, J=2.1 Hz, 1H), 9.25 (s, 1H), 9.10 (dd, J=4.1, 1.5 Hz, 1H), 8.75 (s, 1H), 8.51 (d, J=8.0 Hz, 1H), 8.35 (d, J=8.6 Hz, 1H), 8.24 (s, 1H), 7.85 (dd, J=8.5, 4.2 Hz, 1H), 7.80 (dd, J=8.6, 1.5 Hz, 1H), 4.40-3.64 (m, 6H), 3.39-3.12 (m, 3H), 1.59 (dt, J=14.2, 7.0 Hz, 2H), 1.15 (d, J=5.9 Hz, 3H), 0.90 (t, J=7.4 Hz, 3H).

LCMS (ESI-TOF) m/z 504.1 [M+H⁺] with a purity of >96%.

### Propyl (S)-4-(4-chloro-2-(4-(methylcarbamoyl)phenyl)quinoline-7-carbonyl)-2-methylpiperazine-1-carboxylate (B189)

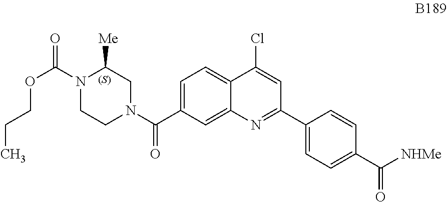

Compound B189 was synthesized according to General Procedure L using propyl (S)-4-(2,4-dichloroquinoline-7-carbonyl)-2-methylpiperazine-1-carboxylate and 4-(N-methylaminocarbonyl)phenylboronic acid as starting materials.

¹H NMR (400 MHz, 80° C., DMSO-d₆) δ 8.46 (s, 1H), 8.41-8.32 (m, 3H), 8.30 (d, J=8.5 Hz, 1H), 8.14 (s, 1H), 8.00 (d, J=8.4 Hz, 2H), 7.75 (dd, J=8.5, 1.3 Hz, 1H), 4.39-3.75 (m, 6H), 3.38-3.10 (m, 3H), 2.84 (d, J=4.5 Hz, 3H), 1.66-1.51 (m, 2H), 1.14 (d, J=5.6 Hz, 3H), 0.89 (t, J=7.4 Hz, 3H).

LCMS (ESI-TOF) m/z 509.2 [M+H⁺] with a purity of >98%.

### Propyl (S)-4-(4-chloro-2-(4-(methylcarbamoyl)phenyl)quinoline-7-carbonyl)-3-methylpiperazine-1-carboxylate (B190)

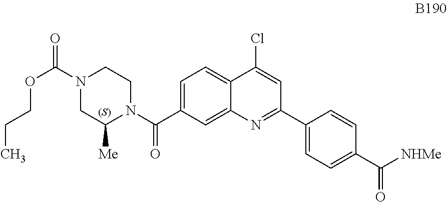

Compound B190 was synthesized according to General Procedure L using propyl (S)-4-(2,4-dichloroquinoline-7-carbonyl)-3-methylpiperazine-1-carboxylate and 4-(N-methylaminocarbonyl)phenylboronic acid as starting materials.

¹H NMR (400 MHz, 80° C., DMSO-d₆) δ 8.45 (s, 1H), 8.39-8.31 (m, 3H), 8.29 (d, J=8.6 Hz, 1H), 8.12 (d, J=1.1 Hz, 1H), 8.00 (d, J=8.4 Hz, 2H), 7.73 (dd, J=8.5, 1.4 Hz, 1H), 4.49-3.73 (m, 6H), 3.32-3.16 (m, 2H), 3.03-2.94 (m, 1H), 2.84 (d, J=4.6 Hz, 3H), 1.67-1.50 (m, 2H), 1.21 (d, J=6.7 Hz, 3H), 0.90 (t, J=7.4 Hz, 3H).

LCMS (ESI-TOF) m/z 509.2 [M+H⁺] with a purity of >98%.

### Propyl 4-(4-chloro-2-(3-fluoro-4-hydroxyphenyl)quinoline-7-carbonyl)piperazine-1-carboxylate (B191)

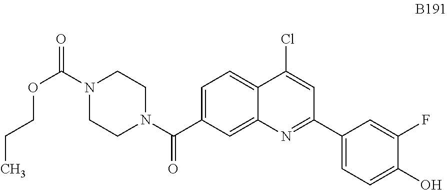 [SMILES: CCCOC(=O)N1CCN(C(=O)c2ccc3c(Cl)cc(-c4ccc(O)c(F)c4)nc3c2)CC1]

Compound B191 was synthesized according to General Procedure L using propyl 4-(2,4-dichloroquinoline-7-carbonyl)piperazine-1-carboxylate and 3-fluoro-4-hydroxyphenylboronic acid as starting materials.

¹H NMR (400 MHz, DMSO-d₆) δ 10.47 (s, 1H), 8.43 (s, 1H), 8.24 (d, J=8.4 Hz, 1H), 8.16 (d, J=12.8 Hz, 1H), 8.09 (s, 1H), 8.04 (d, J=9.1 Hz, 1H), 7.71 (d, J=8.6 Hz, 1H), 7.11 (t, J=8.7 Hz, 1H), 3.98 (t, J=6.5 Hz, 2H), 3.79-3.37 (m, 8H), 1.59 (d, J=7.1 Hz, 2H), 0.89 (t, J=7.0 Hz, 3H).

LCMS (ESI-TOF) m/z 472.1 [M+H⁺] with a purity of >95%.

### Propyl (S)-4-(4-chloro-2-(3-fluoro-4-hydroxyphenyl)quinoline-7-carbonyl)-2-methylpiperazine-1-carboxylate (B192)

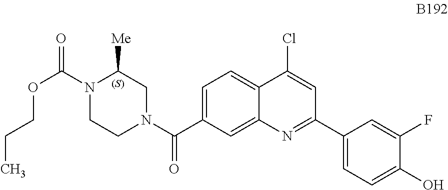

Compound B192 was synthesized according to General Procedure L using propyl (S)-4-(2,4-dichloroquinoline-7-carbonyl)-2-methylpiperazine-1-carboxylate and 3-fluoro-4-hydroxyphenylboronic acid as starting materials.

¹H NMR (400 MHz, 80° C., DMSO-d₆) δ 10.14 (s, 1H), 8.33 (s, 1H), 8.24 (d, J=8.5 Hz, 1H), 8.14-8.07 (m, 1H), 8.05 (s, 1H), 7.99 (d, J=8.5 Hz, 1H), 7.68 (d, J=9.9 Hz, 1H), 7.10 (t, J=8.8 Hz, 1H), 4.49-3.64 (m, 6H), 3.39-3.10 (m, 3H), 1.66-1.49 (m, 2H), 1.13 (d, J=5.9 Hz, 3H), 0.89 (t, J=7.4 Hz, 3H).

LCMS (ESI-TOF) m/z 486.1 [M+H⁺] with a purity of >98%.

### Propyl (S)-4-(4-chloro-2-(1-methyl-1H-pyrrol-3-yl)quinoline-7-carbonyl)-2-methylpiperazine-1-carboxylate (B193)

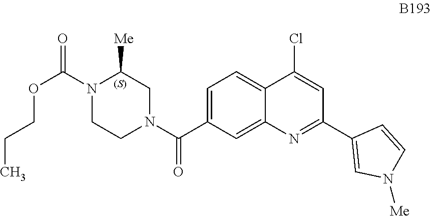

Compound B193 was synthesized according to General Procedure L using propyl (S)-4-(2,4-dichloroquinoline-7-carbonyl)-2-methylpiperazine-1-carboxylate and 1-methylpyrrole-3-boronic acid pinacol ester as starting materials.

¹H NMR (400 MHz, 80° C., DMSO-d₆) δ 8.14 (d, J=8.6 Hz, 1H), 7.98 (s, 1H), 7.88 (s, 1H), 7.64 (s, 1H), 7.55 (d, J=8.3 Hz, 1H), 6.79 (d, J=11.5 Hz, 2H), 4.39-3.59 (m, 9H), 3.38-3.09 (m, 3H), 1.59 (dd, J=14.0, 7.0 Hz, 2H), 1.12 (d, J=6.2 Hz, 3H), 0.89 (t, J=7.4 Hz, 3H).

LCMS (ESI-TOF) m/z 455.1 [M+H⁺] with a purity of >99%.

### Propyl (S)-4-(4-chloro-2-(1-methyl-1H-pyrrol-3-yl)quinoline-7-carbonyl)-3-methylpiperazine-1-carboxylate (B194)

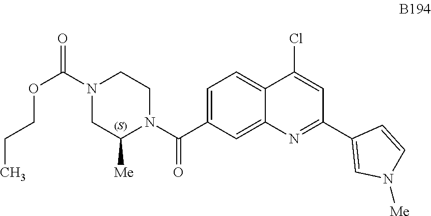

Compound B194 was synthesized according to General Procedure L using propyl (S)-4-(2,4-dichloroquinoline-7-carbonyl)-3-methylpiperazine-1-carboxylate and 1-methylpyrrole-3-boronic acid pinacol ester as starting materials.

¹H NMR (400 MHz, 80° C., DMSO-d₆) δ 8.13 (d, J=8.3 Hz, 1H), 7.97 (s, 1H), 7.86 (d, J=1.1 Hz, 1H), 7.64 (s, 1H), 7.53 (dd, J=8.4, 1.6 Hz, 1H), 6.79 (dt, J=4.5, 2.7 Hz, 2H), 4.50-3.55 (m, 9H), 3.30-3.12 (m, 2H), 3.02-2.93 (m, 1H), 1.59 (dd, J=14.0, 6.6 Hz, 2H), 1.19 (d, J=6.8 Hz, 3H), 0.89 (t, J=7.4 Hz, 3H).

LCMS (ESI-TOF) m/z 455.1 [M+H⁺] with a purity of >99%.

### Propyl 4-(4-chloro-2-(1-methyl-1H-indazol-6-yl)quinoline-7-carbonyl)piperazine-1-carboxylate (B195)

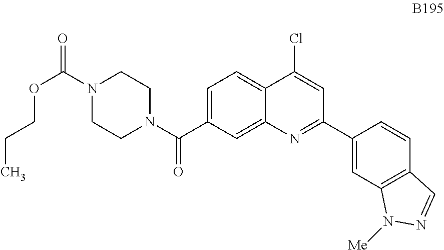 [SMILES: CCCOC(=O)N1CCN(C(=O)c2ccc3c(Cl)cc(-c4ccc5cnn(C)c5c4)nc3c2)CC1]

Compound B195 was synthesized according to General Procedure L using propyl 4-(2,4-dichloroquinoline-7-carbonyl)piperazine-1-carboxylate and 1-methyl-1H-indazol-6-ylboronic acid as starting materials.

¹H NMR (400 MHz, DMSO-d₆) 6 8.70 (s, 1H), 8.64 (s, 1H), 8.30 (d, J=8.6 Hz, 1H), 8.20 (d, J=10.3 Hz, 1H), 8.19 (s, 1H), 8.13 (s, 1H), 7.92 (d, J=8.6 Hz, 1H), 7.77 (d, J=8.6 Hz, 1H), 4.19 (s, 3H), 3.98 (t, J=6.6 Hz, 2H), 3.77-3.39 (m, 8H), 1.71-1.52 (m, 2H), 0.89 (t, J=7.3 Hz, 3H).

LCMS (ESI-TOF) m/z 492.1 [M+H⁺] with a purity of >99%.

### Propyl 4-(4-chloro-2-(2-methyl-2H-indazol-6-yl)quinoline-7-carbonyl)piperazine-1-carboxylate (B196)

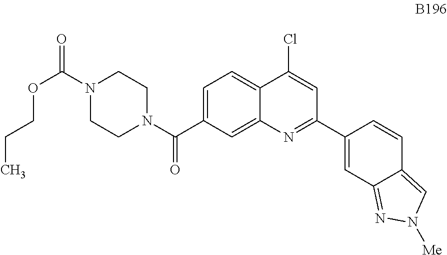 [SMILES: CCCOC(=O)N1CCN(C(=O)c2ccc3c(Cl)cc(-c4ccc5cn(C)nc5c4)nc3c2)CC1]

Compound B196 was synthesized according to General Procedure L using propyl 4-(2,4-dichloroquinoline-7-carbonyl)piperazine-1-carboxylate and 2-methyl-2H-indazol -6-ylboronic acid as starting materials.

¹H NMR (400 MHz, DMSO-d₆) δ 8.60 (s, 2H), 8.42 (s, 1H), 8.28 (d, J=8.4 Hz, 1H), 8.16 (d, J=0.8 Hz, 1H), 8.09 (dd, J=8.8, 0.8 Hz, 1H), 7.87 (d, J=8.8 Hz, 1H), 7.74 (dd, J=8.8, 0.8 Hz, 1H), 4.23 (s, 3H), 3.98 (t, J=6.8 Hz, 2H), 3.70-3.43 (m, 8H), 1.59 (dd, J=14.4, 6.8 Hz, 2H), 0.89 (t, J=7.2 Hz, 3H).

LCMS (ESI-TOF) m/z 492.1 [M+H⁺] with a purity of >96%.

### Propyl (R)-4-(4-chloro-2-(3-methyl-1H-indazol-5-yl)quinoline-7-carbonyl)-2-methylpiperazine-1-carboxylate (B197)

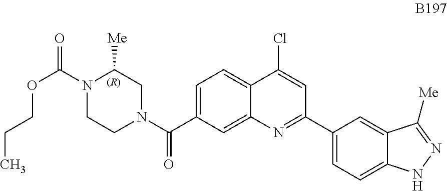

Compound B197 was synthesized according to General Procedure L using propyl (R)-4-(2,4-dichloroquinoline-7-carbonyl)-2-methylpiperazine-1-carboxylate and 3-methyl-1H-indazole-5-boronic acid as starting materials.

¹H NMR (400 MHz, 80° C., DMSO-d₆) δ 12.64 (s, 1H), 8.68 (s, 1H), 8.52 (s, 1H), 8.36 (d, J=8.9 Hz, 1H), 8.26 (d, J=8.5 Hz, 1H), 8.10 (s, 1H), 7.68 (d, J=8.6 Hz, 1H), 7.59 (d, J=8.7 Hz, 1H), 4.41-3.65 (m, 6H), 3.40-3.12 (m, 3H), 2.60 (s, 3H), 1.60 (dt, J=14.2, 7.3 Hz, 2H), 1.14 (d, J=6.4 Hz, 3H), 0.89 (t, J=7.4 Hz, 3H).

LCMS (ESI-TOF) m/z 506.1 [M+H⁺] with a purity of >96%.

### Propyl (S)-4-(4-chloro-2-(3-methyl-1H-indazol-5-yl)quinoline-7-carbonyl)-3-methylpiperazine-1-carboxylate (B198)

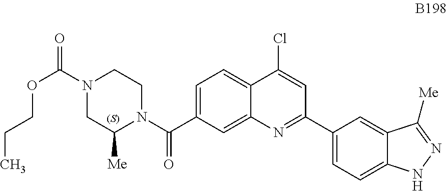

Compound B198 was synthesized according to General Procedure L using propyl (S)-4-(2,4-dichloroquinoline-7-carbonyl)-3-methylpiperazine-1-carboxylate and 3-methyl-1H-indazole-5-boronic acid as starting materials.

¹H NMR (400 MHz, 80° C., DMSO-d₆) δ 12.63 (s, 1H), 8.67 (s, 1H), 8.51 (s, 1H), 8.36 (d, J=8.8 Hz, 1H), 8.26 (d, J=8.4 Hz, 1H), 8.09 (s, 1H), 7.67 (d, J=8.5 Hz, 1H), 7.59 (d, J=8.9 Hz, 1H), 4.63-3.67 (m, 6H), 3.35-3.14 (m, 3H), 3.04-2.94 (m, 1H), 1.60 (dd, J=14.1, 6.9 Hz, 2H), 1.21 (d, J=6.7 Hz, 3H), 0.90 (t, J=7.4 Hz, 3H).

LCMS (ESI-TOF) m/z 506.1 [M+H⁺] with a purity of >99%.

### Propyl (R)-4-(4-chloro-2-(3-methyl-1H-pyrazolo[3,4-b]pyridin-5-yl)quinoline-7-carbonyl)-2-methylpiperazine-1-carboxylate (B199)

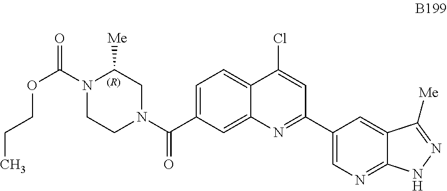

Compound B199 was synthesized according to General Procedure L using propyl (R)-4-(2,4-dichloroquinoline-7-carbonyl)-2-methylpiperazine-1-carboxylate and 3-methyl-1H-pyrazolo [3 ,4-B]pyridine-5-boronic acid pinacol ester as starting materials.

¹H NMR (400 MHz, 80° C., DMSO-d₆) δ 13.25 (s, 1H), 9.45 (d, J=2.0 Hz, 1H), 9.08 (d, J=2.0 Hz, 1H), 8.58 (s, 1H), 8.29 (d, J=8.6 Hz, 1H), 8.15 (s, 1H), 7.72 (dd, J=8.5, 1.5 Hz, 1H), 4.56-3.60 (m, 6H), 3.39-3.11 (m, 3H), 1.67-1.52 (m, 2H), 1.15 (d, J=5.7 Hz, 3H), 0.89 (t, J=7.4 Hz, 3H).

LCMS (ESI-TOF) m/z 507.1 [M+H⁺] with a purity of >99%.

### Propyl (S)-4-(4-chloro-2-(3-methyl-1H-pyrazolo[3,4-b]pyridin-5-yl)quinoline-7-carbonyl)-3-methylpiperazine-1-carboxylate (B200)

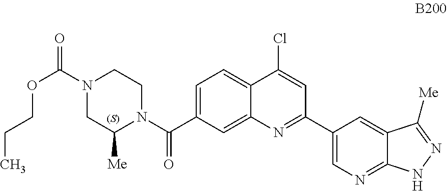

Compound B200 was synthesized according to General Procedure L using propyl (S)-4-(2,4-dichloroquinoline-7-carbonyl)-3-methylpiperazine-1-carboxylate and 3-methyl-1H-pyrazolo [3 ,4-B]pyridine-5-boronic acid pinacol ester as starting materials.

¹H NMR (400 MHz, 80° C., DMSO-d₆) δ 13.24 (s, 1H), 9.45 (d, J=2.1 Hz, 1H), 9.07 (d, J=2.1 Hz, 1H), 8.56 (s, 1H), 8.28 (d, J=8.6 Hz, 1H), 8.13 (d, J=1.1 Hz, 1H), 7.70 (dd, J=8.5, 1.6 Hz, 1H), 4.55-3.66 (m, 6H), 3.35-3.15 (m, 2H), 3.03-2.89 (m, 1H), 1.68-1.50 (m, 2H), 1.22 (d, J=6.7 Hz, 3H), 0.90 (t, J=7.4 Hz, 3H).

LCMS (ESI-TOF) m/z 507.1 [M+H⁺] with a purity of >99%.

### Propyl 4-(4-chloro-2-(3,5-difluoro-4-hydroxyphenyl)quinoline-7-carbonyl)piperazine-1-carboxylate (B201)

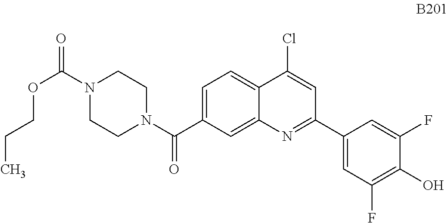 [SMILES: CCCOC(=O)N1CCN(C(=O)c2ccc3c(Cl)cc(-c4cc(F)c(O)c(F)c4)nc3c2)CC1]

Compound B201 was synthesized according to General Procedure L using propyl 4-(2,4-dichloroquinoline-7-carbonyl)piperazine-1-carboxylate and 3,5-difluoro-4-hydroxyphenylboronic acid as starting materials.

¹H NMR (400 MHz, DMSO-d₆) 6 10.85 (s, 1H), 8.51 (s, 1H), 8.26 (d, J=8.5 Hz, 1H), 8.16-8.04 (m, 3H), 7.73 (d, J=8.5 Hz, 1H), 3.98 (t, J=6.5 Hz, 2H), 3.84-3.34 (m, 8H), 1.59 (d, J=6.6 Hz, 2H), 0.90 (d, J=7.6 Hz, 3H).

LCMS (ESI-TOF) m/z 490.1 [M+H⁺] with a purity of >96%.

### Propyl 4-(4-chloro-2-(4-hydroxy-3-methylphenyl)quinoline-7-carbonyl)piperazine-1-carboxylate (B202)

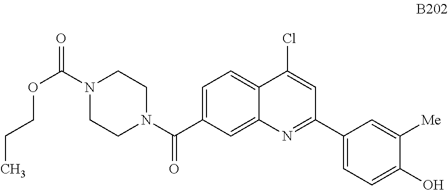 [SMILES: CCCOC(=O)N1CCN(C(=O)c2ccc3c(Cl)cc(-c4ccc(O)c(C)c4)nc3c2)CC1]

Compound B202 was synthesized according to General Procedure L using propyl 4-(2,4-dichloroquinoline-7-carbonyl)piperazine-1-carboxylate and 4-hydroxy-3-methylphenylboronic acid as starting materials.

¹H NMR (400 MHz, DMSO-d₆) δ 9.90 (s, 1H), 8.36 (s, 1H), 8.23 (d, J=8.5 Hz, 1H), 8.11 (s, 1H), 8.07 (s, 1H), 8.02 (d, J=8.6 Hz, 1H), 7.73-7.64 (m, 1H), 6.94 (d, J=8.5 Hz, 1H), 3.98 (t, J=6.6 Hz, 2H), 3.77-3.39 (m, 8H), 2.24 (s, 3H), 1.59 (dd, J=13.6, 6.6 Hz, 2H), 0.90 (t, J=7.0 Hz, 3H).

LCMS (ESI-TOF) m/z 468.1 [M+H⁺] with a purity of >97%.

### Propyl 4-(2-(benzofuran-5-yl)-4-chloroquinoline-7-carbonyl)piperazine-1-carboxylate (B203)

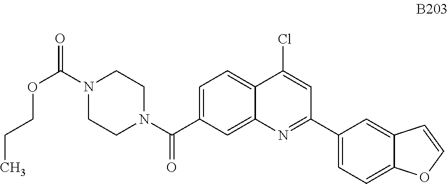 [SMILES: CCCOC(=O)N1CCN(C(=O)c2ccc3c(Cl)cc(-c4ccc5occc5c4)nc3c2)CC1]

Compound B203 was synthesized according to General Procedure L using propyl 4-(2,4-dichloroquinoline-7-carbonyl)piperazine-1-carboxylate and benzofuran-5-boronic acid as starting materials.

¹H NMR (400 MHz, DMSO-d₆) δ 8.65 (s, 1H), 8.55 (s, 1H), 8.31 (dd, J=20.6, 8.3 Hz, 2H), 8.13 (d, J=17.1 Hz, 2H), 7.76 (dd, J=16.1, 8.7 Hz, 2H), 7.11 (s, 1H), 3.98 (t, J=6.5 Hz, 2H), 3.77-3.37 (m, 8H), 1.59 (dd, J=14.1, 6.8 Hz, 2H), 0.89 (t, J=6.5 Hz, 3H).

LCMS (ESI-TOF) m/z 478.1 [M+H⁺] with a purity of >99%.

### Propyl 4-(4-chloro-2-(3-fluoro-4-(methylcarbamoyl)phenyl)quinoline-7-carbonyl)piperazine-1-carboxylate (B204)

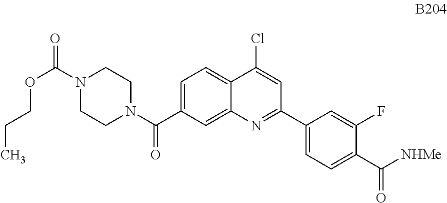 [SMILES: CCCOC(=O)N1CCN(C(=O)c2ccc3c(Cl)cc(-c4ccc(C(=O)NC)c(F)c4)nc3c2)CC1]

Compound B204 was synthesized according to General Procedure L using propyl 4-(2,4-dichloroquinoline-7-carbonyl)piperazine-1-carboxylate and 3-fluoro-4-(N-methylaminocarbonyl)phenylboronic acid as starting materials.

¹H NMR (400 MHz, DMSO-d₆) δ 8.60 (s, 1H), 8.36 (s, 1H), 8.32-8.22 (m, 3H), 8.19 (s, 1H), 7.86-7.75 (m, 2H), 3.98 (t, J=6.6 Hz, 2H), 3.74-3.36 (m, 8H), 2.82 (d, J=4.5 Hz, 3H), 1.59 (dd, J=13.2, 6.3 Hz, 2H), 0.89 (t, J=7.4 Hz, 3H).

LCMS (ESI-TOF) m/z 513.1 [M+H⁺] with a purity of >96%.

### Propyl 4-(4-chloro-2-(4-(cyclopropylcarbamoyl)-3-fluorophenyl)quinoline-7-carbonyl)piperazine-1-carboxylate (B205)

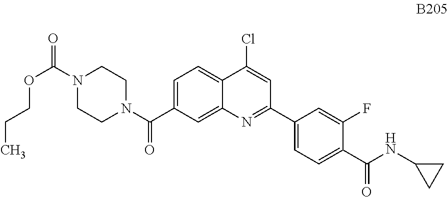 [SMILES: CCCOC(=O)N1CCN(C(=O)c2ccc3c(Cl)cc(-c4ccc(C(=O)NC5CC5)c(F)c4)nc3c2)CC1]

Compound B205 was synthesized according to General Procedure L using propyl 4-(2,4-dichloroquinoline-7-carbonyl)piperazine-1-carboxylate and 3-fluoro-4-(N-cyclopropylaminocarbonyl)phenylboronic acid as starting materials.

¹H NMR (400 MHz, DMSO-d₆) δ 8.60 (s, 1H), 8.50 (s, 1H), 8.30 (d, J=8.5 Hz, 1H), 8.28-8.21 (m, 2H), 8.19 (s, 1H), 7.79 (d, J=8.5 Hz, 1H), 7.74 (t, J=7.8 Hz, 1H), 3.98 (t, J=6.4 Hz, 2H), 3.80-3.36 (m, 8H), 2.93-2.81 (m, 1H), 1.59 (dd, J=12.4, 5.4 Hz, 2H), 0.89 (t, J=7.1 Hz, 3H), 0.81-0.65 (m, 2H), 0.58 (s, 2H).

LCMS (ESI-TOF) m/z 539.1 [M+H⁺] with a purity of >96%.

### Propyl 4-(4-chloro-2-(4-(ethylcarbamoyl)phenyl)quinoline-7-carbonyl)piperazine-1-carboxylate(B206)

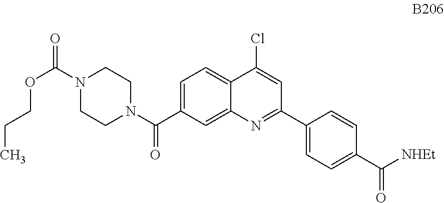

Compound B206 was synthesized according to General Procedure L using propyl 4-(2,4-dichloroquinoline-7-carbonyl)piperazine-1-carboxylate and 4-(N-ethylaminocarbonyl)phenylboronic acid as starting materials.

¹H NMR (400 MHz, DMSO-d₆) δ 8.62 (t, J=5.4 Hz, 1H), 8.57 (s, 1H), 8.42 (d, J=8.4 Hz, 2H), 8.30 (d, J=8.5 Hz, 1H), 8.18 (s, 1H), 8.03 (d, J=8.4 Hz, 2H), 7.78 (d, J=8.6 Hz, 1H), 3.98 (t, J=6.5 Hz, 2H), 3.74-3.39 (m, 8H), 3.37-3.32 (m, 2H), 1.64-1.52 (m, 2H), 1.16 (t, J=7.2 Hz, 3H), 0.89 (t, J=7.4 Hz, 3H).

LCMS (ESI-TOF) m/z 509.1 [M+H⁺] with a purity of >97%.

### Propyl 4-(4-chloro-2-(1-methyl-1H-benzo[d]imidazol-6-yl)quinoline-7-carbonyl)piperazine-1-carboxylate (B207)

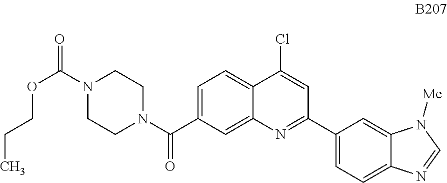 [SMILES: CCCOC(=O)N1CCN(C(=O)c2ccc3c(Cl)cc(-c4ccc5ncn(C)c5c4)nc3c2)CC1]

Compound B207 was synthesized according to General Procedure L using propyl 4-(2,4-dichloroquinoline-7-carbonyl)piperazine-1-carboxylate and 1-methyl-1H-benzoimidazole-6-boronic acid as starting materials.

¹H NMR (400 MHz, 80° C., DMSO-d₆) δ 8.52 (s, 2H), 8.30-8.21 (m, 3H), 8.14 (d, J=1.0 Hz, 1H), 7.78 (d, J=8.7 Hz, 1H), 7.71 (dd, J=8.5, 1.5 Hz, 1H), 4.00 (t, J=6.5 Hz, 2H), 3.96 (s, 3H), 3.69-3.40 (m, 8H), 1.60 (dd, J=14.2, 7.0 Hz, 2H), 0.90 (t, J=7.4 Hz, 3H).

LCMS (ESI-TOF) m/z 492.1 [M+H⁺] with a purity of >95%.

### Propyl 4-(4-chloro-2-(2-methylbenzo[d]thiazol-6-yl)quinoline-7-carbonyl)piperazine-1-carboxylate (B208)

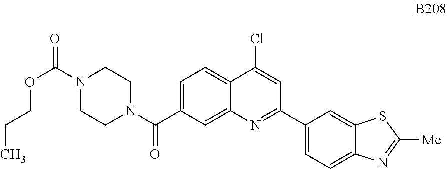 [SMILES: CCCOC(=O)N1CCN(C(=O)c2ccc3c(Cl)cc(-c4ccc5nc(C)sc5c4)nc3c2)CC1]

Compound B208 was synthesized according to General Procedure L using propyl 4-(2,4-dichloroquinoline-7-carbonyl)piperazine-1-carboxylate and 2-methylbenzothiazole-6-boronic acid as starting materials.

¹H NMR (400 MHz, DMSO-d₆) δ 9.03 (d, J=1.1 Hz, 1H), 8.58 (s, 1H), 8.48 (dd, J=8.5, 1.5 Hz, 1H), 8.29 (d, J=8.5 Hz, 1H), 8.16 (d, J=0.7 Hz, 1H), 8.07 (d, J=8.6 Hz, 1H), 7.76 (dd, J=8.6, 1.1 Hz, 1H), 3.98 (t, J=6.5 Hz, 2H), 3.79-3.40 (m, 8H), 2.86 (s, 3H), 1.59 (dd, J=14.0, 6.9 Hz, 2H), 0.89 (t, J=7.3 Hz, 3H).

LCMS (ESI-TOF) m/z 509.1 [M+H⁺] with a purity of >99%.

### Propyl 4-(4-chloro-2-(1H-pyrrol-3-yl)quinoline-7-carbonyl)piperazine-1-carboxylate (B209)

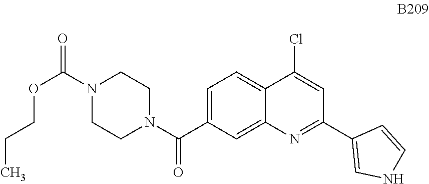 [SMILES: CCCOC(=O)N1CCN(C(=O)c2ccc3c(Cl)cc(-c4cc[nH]c4)nc3c2)CC1]

Compound B209 was synthesized according to General Procedure L using propyl 4-(2,4-dichloroquinoline-7-carbonyl)piperazine-1-carboxylate and 1H-pyrrole-3-boronic acid pinacol ester as starting materials.

¹H NMR (400 MHz, DMSO-d₆) δ 11.31 (s, 1H), 8.15 (d, J=8.4 Hz, 1H), 8.14 (s, 1H), 7.92 (s, 1H), 7.76 (s, 1H), 7.58 (d, J=8.7 Hz, 1H), 6.89 (d, J=1.7 Hz, 1H), 6.84 (s, 1H), 3.98 (t, J=6.5 Hz, 2H), 3.77-3.44 (m, 8H), 1.59 (d, J=8.0 Hz, 2H), 0.89 (t, J=7.2 Hz, 3H).

LCMS (ESI-TOF) m/z 427.1 [M+H⁺] with a purity of >97%.

### Propyl 4-(4-chloro-2-(4-(cyclopropylcarbamoyl)phenyl)quinoline-7-carbonyl)piperazine-1-carboxylate (B210)

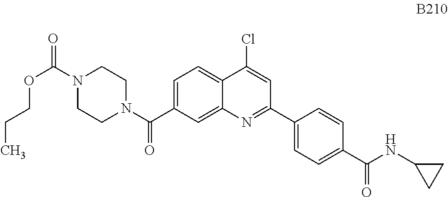 [SMILES: CCCOC(=O)N1CCN(C(=O)c2ccc3c(Cl)cc(-c4ccc(C(=O)NC5CC5)cc4)nc3c2)CC1]

Compound B210 was synthesized according to General Procedure L using propyl 4-(2,4-dichloroquinoline-7-carbonyl)piperazine-1-carboxylate and 4-(N-cyclopropylaminocarbonyl)phenylboronic acid as starting materials.

¹H NMR (400 MHz, DMSO-d₆) δ 8.59 (d, J=4.0 Hz, 1H), 8.57 (s, 1H), 8.41 (d, J=8.1 Hz, 2H), 8.30 (d, J=8.4 Hz, 1H), 8.18 (s, 1H), 8.01 (d, J=8.1 Hz, 2H), 7.78 (d, J=8.6 Hz, 1H), 3.98 (t, J=6.5 Hz, 2H), 3.84-3.37 (m, 8H), 2.89 (d, J=4.6 Hz, 1H), 1.59 (dd, J=11.8, 5.6 Hz, 2H), 0.89 (t, J=7.4 Hz, 3H), 0.73 (d, J=5.9 Hz, 2H), 0.62 (d, J=2.1 Hz, 2H).

LCMS (ESI-TOF) m/z 521.2 [M+H⁺] with a purity of >96%.

### Propyl 4-(4-chloro-2-(3-methyl-1H-indol-5-yl)quinoline-7-carbonyl)piperazine-1-carboxylate (B211)

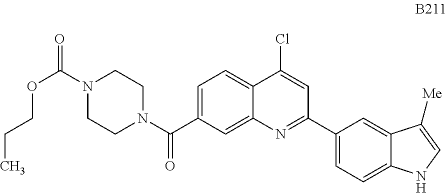 [SMILES: CCCOC(=O)N1CCN(C(=O)c2ccc3c(Cl)cc(-c4ccc5[nH]cc(C)c5c4)nc3c2)CC1]

Compound B211 was synthesized according to General Procedure L using propyl 4-(2,4-dichloroquinoline-7-carbonyl)piperazine-1-carboxylate and 3-methylindole-5-lboronic acid pinacol ester as starting materials.

¹H NMR (400 MHz, DMSO-d₆) δ 10.99 (s, 1H), 8.55 (s, 1H), 8.51 (s, 1H), 8.24 (d, J=8.5 Hz, 1H), 8.15 (d, J=8.5 Hz, 1H), 8.11 (s, 1H), 7.68 (d, J=8.5 Hz, 1H), 7.48 (d, J=8.6 Hz, 1H), 7.20 (s, 1H), 3.98 (t, J=6.6 Hz, 2H), 3.79-3.37 (m, 8H), 2.37 (s, 3H), 1.59 (dd, J=13.8, 6.9 Hz, 2H), 0.89 (t, J=7.1 Hz, 3H).

LCMS (ESI-TOF) m/z 491.1 [M+H⁺] with a purity of >99%.

### Propyl 4-(4-chloro-2-(2-methylbenzo[d]oxazol-6-yl)quinoline-7-carbonyl)piperazine-1-carboxylate (B212)

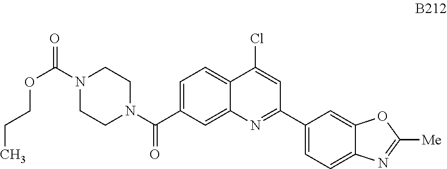 [SMILES: CCCOC(=O)N1CCN(C(=O)c2ccc3c(Cl)cc(-c4ccc5nc(C)oc5c4)nc3c2)CC1]

Compound B212 was synthesized according to General Procedure L using propyl 4-(2,4-dichloroquinoline-7-carbonyl)piperazine-1-carboxylate and (2-methyl-1,3-benzoxazol-6-yl)boronic acid as starting materials.

¹H NMR (400 MHz, DMSO-d₆) δ 8.62 (s, 1H), 8.60 (s, 1H), 8.39 (d, J=8.4 Hz, 1H), 8.29 (d, J=8.5 Hz, 1H), 8.16 (s, 1H), 7.83 (d, J=8.4 Hz, 1H), 7.76 (d, J=8.5 Hz, 1H), 3.98 (t, J=6.6 Hz, 2H), 3.83-3.41 (m, 8H), 2.68 (s, 3H), 1.59 (dd, J=15.3, 8.7 Hz, 2H), 0.89 (t, J=7.3 Hz, 3H).

LCMS (ESI-TOF) m/z 493.1 [M+H⁺] with a purity of >99%.

### Propyl 4-(2-(benzo[d]oxazol-5-yl)-4-chloroquinoline-7-carbonyl)piperazine-1-carboxylate (B213)

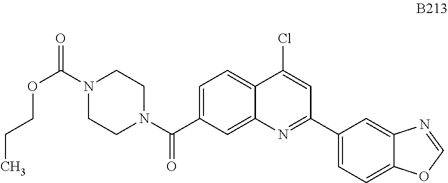 [SMILES: CCCOC(=O)N1CCN(C(=O)c2ccc3c(Cl)cc(-c4ccc5ocnc5c4)nc3c2)CC1]

Compound B213 was synthesized according to General Procedure L using propyl 4-(2,4-dichloroquinoline-7-carbonyl)piperazine-1-carboxylate and 1,3-benzoxazole-5-boronic acid as starting materials.

¹H NMR (400 MHz, DMSO-d₆) δ 8.87 (s, 1H), 8.78 (s, 1H), 8.64 (s, 1H), 8.49 (d, J=8.4 Hz, 1H), 8.29 (d, J=8.5 Hz, 1H), 8.18 (s, 1H), 7.97 (d, J=8.6 Hz, 1H), 7.76 (d, J=8.5 Hz, 1H), 3.98 (t, J=6.5 Hz, 2H), 3.76-3.38 (m, 8H), 1.59 (dd, J=12.8, 5.9 Hz, 2H), 0.89 (t, J=6.9 Hz, 3H).

LCMS (ESI-TOF) m/z 479.1 [M+H⁺] with a purity of >96%.

### Propyl 4-(4-chloro-2-(1,2,5-trimethyl-1H-pyrrol-3-yl)quinoline-7-carbonyl)piperazine-1-carboxylate (B214)

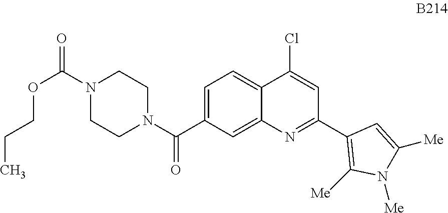 [SMILES: CCCOC(=O)N1CCN(C(=O)c2ccc3c(Cl)cc(-c4cc(C)n(C)c4C)nc3c2)CC1]

Compound B214 was synthesized according to General Procedure L using propyl 4-(2,4-dichloroquinoline-7-carbonyl)piperazine-1-carboxylate and 1,2,5-trimethylpyrrole-3-boronic acid pinacol ester as starting materials.

¹H NMR (400 MHz, DMSO-d₆) δ 8.12 (d, J=8.4 Hz, 1H), 7.90 (s, 1H), 7.82 (s, 1H), 7.54 (d, J=8.3 Hz, 1H), 6.40 (s, 1H), 3.99 (t, J=6.5 Hz, 2H), 3.65-3.34 (m, 8H), 3.45 (s, 3H), 2.71 (s, 3H), 2.22 (s, 3H), 1.60 (dd, J=14.0, 7.0 Hz, 2H), 0.89 (t, J=7.4 Hz, 3H).

LCMS (ESI-TOF) m/z 469.2 [M+H⁺] with a purity of >97%.

### Propyl (R)-4-(4-chloro-2-(1-methyl-1H-pyrazol-4-yl)quinoline-7-carbonyl)-2-methylpiperazine-1-carboxylate (B215)

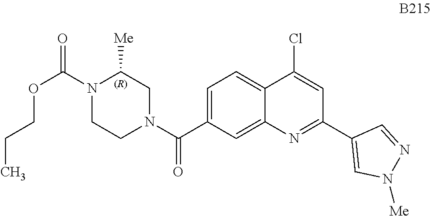

Compound B215 was synthesized according to General Procedure L using propyl (R)-4-(2,4-dichloroquinoline-7-carbonyl)-2-methylpiperazine-1-carboxylate and 1-methyl-1H-pyrazole-4-boronic acid as starting materials.

¹H NMR (400 MHz, 80° C., DMSO-d₆) δ 8.47 (s, 1H), 8.19 (d, J=8.5 Hz, 1H), 8.17 (s, 1H), 8.10 (s, 1H), 7.94 (s, 1H), 7.62 (dd, J=8.5, 1.3 Hz, 1H), 4.34-3.61 (m, 9H), 3.38-3.09 (m, 3H), 1.67-1.50 (m, 2H), 1.13 (d, J=6.4 Hz, 3H), 0.89 (t, J=7.4 Hz, 3H).

LCMS (ESI-TOF) m/z 456.1 [M+H⁺] with a purity of >98%.

### Propyl (R)-4-(4-chloro-2-(3-fluoro-4-(methylcarbamoyl)phenyl)quinoline-7-carbonyl)-2-methylpiperazine-1-carboxylate (B216)

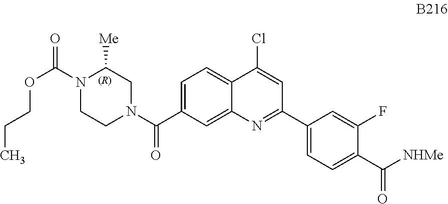

Compound B216 was synthesized according to General Procedure L using propyl (R)-4-(2,4-dichloroquinoline-7-carbonyl)-2-methylpiperazine-1-carboxylate and 3-fluoro-4-(methylcarbamoyl)phenylboronic acid as starting materials.

¹H NMR (400 MHz, 80° C., DMSO-d₆) δ 8.48 (s, 1H), 8.30 (d, J=8.4 Hz, 1H), 8.26-8.13 (m, 3H), 8.08 (br s, 1H), 7.86-7.72 (m, 2H), 4.30-3.78 (m, 6H), 3.39-3.09 (m, 3H), 2.84 (d, J=4.4 Hz, 3H), 1.60 (dd, J=13.9, 6.9 Hz, 2H), 1.15 (s, 3H), 0.89 (t, J=7.3 Hz, 3H).

LCMS (ESI-TOF) m/z 527.1 [M+H⁺] with a purity of >99%.

### Propyl (R)-4-(4-chloro-2-(3,5-difluoro-4-hydroxyphenyl)quinoline-7-carbonyl)-2-methylpiperazine-1-carboxylate (B217)

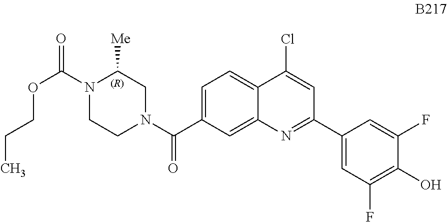

Compound B217 was synthesized according to General Procedure L using propyl (R)-4-(2,4-dichloroquinoline-7-carbonyl)-2-methylpiperazine-1-carboxylate and 3,5-difluoro-4-hydroxyphenylboronic acid as starting materials.

¹H NMR (400 MHz, 80° C., DMSO-d₆) δ 8.39 (s, 1H), 8.25 (d, J=8.6 Hz, 1H), 8.08 (s, 1H), 8.00 (d, J=9.6 Hz, 2H), 7.70 (d, J=8.8 Hz, 1H), 4.36-3.66 (m, 6H), 3.37-3.12 (m, 3H), 1.68-1.45 (m, 2H), 1.14 (d, J=6.2 Hz, 3H), 0.89 (t, J=7.4 Hz, 3H).

LCMS (ESI-TOF) m/z 504.1 [M+H⁺] with a purity of >97%.

### Propyl (R)-4-(4-chloro-2-(4-hydroxy-3-methylphenyl)quinoline-7-carbonyl)-2-methylpiperazine-1-carboxylate (B218)

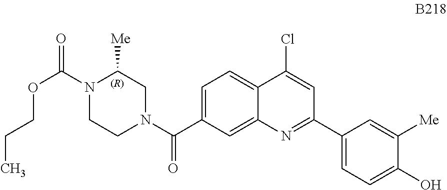

Compound B218 was synthesized according to General Procedure L using propyl (R)-4-(2,4-dichloroquinoline-7-carbonyl)-2-methylpiperazine-1-carboxylate and 4-hydroxy-3-methylphenylboronic acid as starting materials.

¹H NMR (400 MHz, 80° C., DMSO-d₆) δ 8.25 (s, 1H), 8.22 (d, J=8.5 Hz, 1H), 8.04 (d, J=9.9 Hz, 2H), 7.96 (dd, J=8.4, 2.0 Hz, 1H), 7.64 (d, J=8.2 Hz, 1H), 6.93 (d, J=8.4 Hz, 1H), 4.37-3.69 (m, 6H), 3.40-3.14 (m, 3H), 2.25 (s, 3H), 1.68-1.48 (m, 2H), 1.14 (d, J=6.4 Hz, 3H), 0.89 (t, J=7.4 Hz, 3H).

LCMS (ESI-TOF) m/z 482.1 [M+H⁺] with a purity of >96%.

### Propyl (S)-4-(4-chloro-2-(1-methyl-1H-pyrazol-4-yl)quinoline-7-carbonyl)-3-methylpiperazine-1-carboxylate (B219)

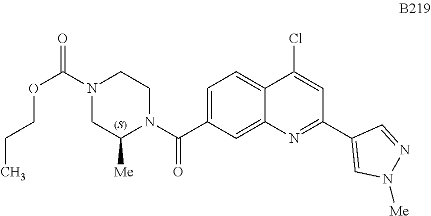

Compound B219 was synthesized according to General Procedure L using propyl (S)-4-(2,4-dichloroquinoline-7-carbonyl)-3-methylpiperazine-1-carboxylate and 1-methyl-1H-pyrazole-4-boronic acid as starting materials.

¹H NMR (400 MHz, 80° C., DMSO-d₆) δ 8.46 (s, 1H), 8.19 (d, J=8.6 Hz, 1H), 8.17 (s, 1H), 8.09 (s, 1H), 7.93 (s, 1H), 7.60 (d, J=8.5 Hz, 1H), 4.45-3.70 (m, 9H), 3.16 (ddd, J=33.3, 16.7, 7.2 Hz, 3H), 1.65-1.48 (m, 2H), 1.20 (d, J=6.7 Hz, 3H), 0.90 (t, J=7.4 Hz, 3H).

LCMS (ESI-TOF) m/z 456.1 [M+H⁺] with a purity of >99%.

### Propyl (S)-4-(4-chloro-2-(3,5-difluoro-4-hydroxyphenyl)quinoline-7-carbonyl)-3-methylpiperazine-1-carboxylate (B220)

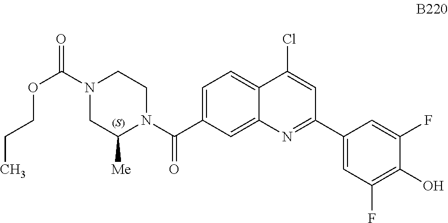

Compound B220 was synthesized according to General Procedure L using propyl (S)-4-(2,4-dichloroquinoline-7-carbonyl)-3-methylpiperazine-1-carboxylate and 3,5-difluoro-4-hydroxyphenylboronic acid as starting materials.

¹H NMR (400 MHz, 80° C., DMSO-d₆) δ 8.39 (s, 1H), 8.25 (d, J=8.5 Hz, 1H), 8.06 (d, J=1.0 Hz, 1H), 8.01 (d, J=9.9 Hz, 2H), 7.68 (dd, J=8.5, 1.4 Hz, 1H), 4.49-3.72 (m, 6H), 3.34-3.16 (m, 3H), 1.66-1.55 (m, 2H), 1.20 (d, J=6.7 Hz, 3H), 0.90 (t, J=7.4 Hz, 3H).

LCMS (ESI-TOF) m/z 504.1 [M+H⁺] with a purity of >98%.

### Propyl 4-(4-chloro-2-(1H-pyrrolo[2,3-b]pyridin-4-yl)quinoline-7-carbonyl)piperazine-1-carboxylate (B221)

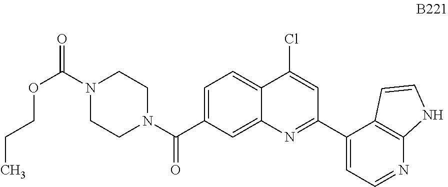 [SMILES: CCCOC(=O)N1CCN(C(=O)c2ccc3c(Cl)cc(-c4ccnc5[nH]ccc45)nc3c2)CC1]

Compound B221 was synthesized according to General Procedure L using propyl 4-(2,4-dichloroquinoline-7-carbonyl)piperazine-1-carboxylate and 1H-pyrrolo[2,3-b]pyridine-4-ylboronic acid pinacol ester as starting materials.

¹H NMR (400 MHz, DMSO-d₆) δ 11.92 (s, 1H), 8.54 (s, 1H), 8.42 (d, J=5.0 Hz, 1H), 8.34 (d, J=8.6 Hz, 1H), 8.28 (s, 1H), 7.85 (d, J=5.1 Hz, 1H), 7.82 (dd, J=8.6, 1.4 Hz, 1H), 7.67 (t, J=2.8 Hz, 1H), 7.34 (d, J=3.2 Hz, 1H), 3.99 (t, J=6.6 Hz, 2H), 3.78-3.37 (m, 8H), 1.59 (dd, J=14.4, 7.4 Hz, 2H), 0.90 (t, J=6.9 Hz, 3H).

LCMS (ESI-TOF) m/z 478.1 [M+H⁺] with a purity of >97%.

### Propyl 4-(4-chloro-2-(2-methyl-1H-benzo[d]imidazol-5-yl)quinoline-7-carbonyl)piperazine-1-carboxylate (B222)

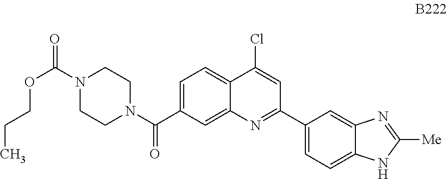 [SMILES: CCCOC(=O)N1CCN(C(=O)c2ccc3c(Cl)cc(-c4ccc5[nH]c(C)nc5c4)nc3c2)CC1]

Compound B222 was synthesized according to General Procedure L using propyl 4-(2,4-dichloroquinoline-7-carbonyl)piperazine-1-carboxylate and (2-methyl-1H-1,3-benzodiazol-6-yl)boronic acid as starting materials.

¹H NMR (400 MHz, DMSO-d₆) δ 12.43 (d, J=20.7 Hz, 1H), 8.52 (s, 1H), 8.44 (d, J=42.3 Hz, 1H), 8.26 (d, J=8.5 Hz, 1H), 8.22-8.08 (m, 2H), 7.71 (d, J=8.8 Hz, 1H), 7.60 (dd, J=32.9, 7.7 Hz, 1H), 3.99 (t, J=6.6 Hz, 2H), 3.79-3.38 (m, 8H), 2.55 (s, 3H), 1.59 (dd, J=14.0, 6.4 Hz, 2H), 0.90 (t, J=7.1 Hz, 3H).

LCMS (ESI-TOF) m/z 492.1 [M+H⁺] with a purity of >97%.

### 1-(4-(4-chloro-2-phenylquinoline-7-carbonyl)piperazin-1-yl)pentan-1-one (B223)

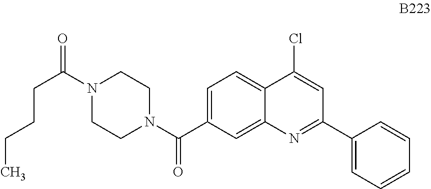 [SMILES: CCCCC(=O)N1CCN(C(=O)c2ccc3c(Cl)cc(-c4ccccc4)nc3c2)CC1]

Step 1: Intermediate from General Procedure K in the synthesis of B002 was subjected to General Procedure C1 with tert-butyl piperazine-1-carboxylate as reagent to afford tert-butyl 4-(4-chloro-2-phenylquinoline-7-carbonyl)piperazine-1-carboxylate.

Step 2: The intermediate from above was dissolved in dichloromethane and trifluoroacetic acid (1:1) and after 10 min, the mixture was concentrated under reduced pressure. The crude material was re-dissolved in ethyl acetate and basified with solid sodium bicarbonate and minimal amount of water. The separated organic layer was dried over anhydrous sodium sulfate, filtered and concentrated under reduced pressure. The crude material was used without further purification.

Step 3: The crude material (88.8 mg, 0.252 mmol) was dissolved in dichloromethane (3 mL) and triethylamine (53 μL, 0.38 mmol, 1.5 equiv). To the mixture was added valeryl chloride (40 μL, 0.337 mmol, 1.3 equiv) and the mixture was quenched with saturated ammonium chloride after 20 min. The organic layer was separated and the aqueous layer was extracted twice with dichloromethane. The combined organic layer was dried over anhydrous sodium sulfate, filtered and concentrated under reduced pressure. The crude material was purified by column chromatography (0-50% ethyl acetate/hexanes) to afford B223 as a white solid (44.6 mg, 41%).

¹H NMR (400 MHz, 80° C., DMSO-d₆) δ 8.49 (s, 1H), 8.33 (d, J=7.4 Hz, 2H), 8.29 (d, J=8.6 Hz, 1H), 8.16 (s, 1H), 7.76 (d, J=8.6 Hz, 1H), 7.64-7.50 (m, 3H), 3.79-3.35 (m, 8H), 2.33 (br s, 2H), 1.48 (br s, 2H), 1.31 (br s, 2H), 0.88 (br s, 3H).

LCMS (ESI-TOF) m/z 436.1 [M+H⁺] with a purity of >99%.

### Propyl 4-(4-chloro-2-(4-((2-methoxyethoxy)methyl)phenyl)quinoline-7-carbonyl)piperazine-1-carboxylate (B224)

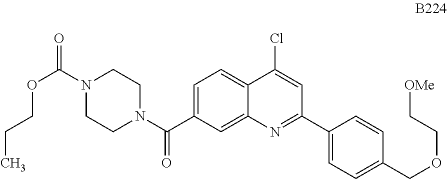 [SMILES: CCCOC(=O)N1CCN(C(=O)c2ccc3c(Cl)cc(-c4ccc(COCCOC)cc4)nc3c2)CC1]

Compound B224 was synthesized according to General Procedure L using propyl 4-(2,4-dichloroquinoline-7-carbonyl)piperazine-1-carboxylate and 4-[(2-methoxyethoxy)methyl]phenylboronic acid as starting materials.

¹H NMR (400 MHz, DMSO-d₆) δ 8.48 (s, 1H), 8.32 (d, J=8.3 Hz, 2H), 8.28 (d, J=8.5 Hz, 1H), 8.14 (d, J=1.1 Hz, 1H), 7.75 (dd, J=8.5, 1.5 Hz, 1H), 7.52 (d, J=8.3 Hz, 2H), 4.60 (s, 2H), 3.98 (t, J=6.6 Hz, 2H), 3.74-3.39 (m, 12H), 3.28 (s, 3H), 1.59 (dd, J=13.8, 6.8 Hz, 2H), 0.89 (t, J=7.4 Hz, 3H).

LCMS (ESI-TOF) m/z 526.1 [M+H⁺] with a purity of >99%.

### Propyl (S)-4-(4-chloro-2-(4-hydroxy-3-methylphenyl)quinoline-7-carbonyl)-3-methylpiperazine-1-carboxylate (B225)

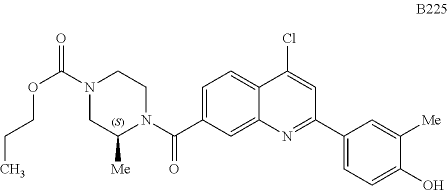

Compound B225 was synthesized according to General Procedure L using propyl (S)-4-(2,4-dichloroquinoline-7-carbonyl)-3-methylpiperazine-1-carboxylate and 4-hydroxy-3-methylphenylboronic acid as starting materials.

¹H NMR (400 MHz, DMSO-d₆) δ 8.35 (s, 1H), 8.22 (d, J=8.5 Hz, 1H), 8.11 (s, 1H), 8.05-7.98 (m, 2H), 7.65 (d, J=8.5 Hz, 1H), 6.93 (d, J=8.4 Hz, 1H), 4.16-2.90 (m, 9H), 2.24 (s, 3H), 1.58 (dd, J=14.0, 7.1 Hz, 2H), 1.18 (s, 3H), 0.89 (t, J=7.2 Hz, 3H).

LCMS (ESI-TOF) m/z 482.1 [M+H⁺] with a purity of >99%.

### 1-(4-(4-chloroquinoline-7-carbonyl)piperazin-1-yl)pentan-1-one (B226)

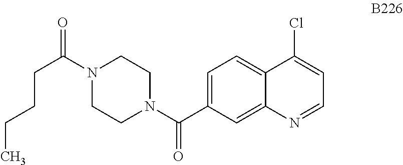 [SMILES: CCCCC(=O)N1CCN(C(=O)c2ccc3c(Cl)ccnc3c2)CC1]

Step 1: According to General Procedure C1, commercially available 4-chloroquinoline-7-carboxylic acid was reacted with tert-butyl-piperazine-1-carboxylate to give tert-butyl 4-(4-chloroquinoline-7-carbonyl)piperazine-1-carboxylate.

Step 2: The intermediate from above was dissolved in dichloromethane and trifluoroacetic acid (1:1) and after 10 min, the mixture was concentrated under reduced pressure. The crude material was re-dissolved in ethyl acetate and basified with solid sodium bicarbonate and minimal amount of water. The separated organic layer was dried over anhydrous sodium sulfate, filtered and concentrated under reduced pressure. The crude material was used without further purification.

Step 3: To a solution of the above residue (86 mg, 0.312 mmol) in dichloromethane (3 mL) was added triethylamine (70 μL, 0.502 mmol, 1.6 equiv) and valeryl chloride (50 μL, 0.421 mmol, 1.3 equiv). The mixture was stirred for 30 min before quenching by the addition of saturated ammonium chloride. The aqueous layer was extracted 3 times with dichloromethane and the combined organics were dried over anhydrous sodium sulfate, filtered and concentrated under reduced pressure. The crude material was purified by column chromatography (0-50% ethyl acetate/hexanes) to afford B226 as colorless oil (56 mg, 50%).

¹H NMR (400 MHz, DMSO-d₆) δ 8.93 (d, J=4.7 Hz, 1H), 8.30 (d, J=8.6 Hz, 1H), 8.13 (d, J=0.4 Hz, 1H), 7.86 (d, J=4.7 Hz, 1H), 7.80 (dd, J=8.5, 1.3 Hz, 1H), 3.82-3.42 (m, 8H), 2.33 (br s, 2H), 1.54-1.38 (m, 2H), 1.31 (br s, 2H), 0.88 (br s, 3H).

LCMS (ESI-TOF) m/z 360.1 [M+H⁺] with a purity of >98%.

### Propyl 4-(2-(2-(aminomethyl)-1,5-dimethyl-1H-pyrrol-3-yl)-4-chloroquinoline-7-carbonyl)piperazine-1-carboxylate (B227)

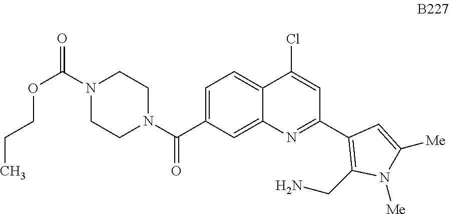 [SMILES: CCCOC(=O)N1CCN(C(=O)c2ccc3c(Cl)cc(-c4cc(C)n(C)c4CN)nc3c2)CC1]

Step 1: Propyl 4-(4-chloro-2-(2-cyano-1,5-dimethyl-1H-pyrrol-3-yl)quinoline-7-carbonyl)piperazine-1-carboxylate was synthesized according to General Procedure L using propyl 4-(2,4-dichloroquinoline-7-carbonyl)piperazine-1-carboxylate and 2-cyano-1,5-dimethylpyrrole-3-boronic acid pinacol ester as starting materials.

Step 2: Compound B227 was synthesized according to General Procedure D using the above intermediate.

¹H NMR (400 MHz, DMSO-d₆) δ 8.14 (d, J=8.5 Hz, 1H), 7.97 (s, 1H), 7.92 (s, 1H), 7.58 (d, J=8.4 Hz, 1H), 6.48 (s, 1H), 4.07 (s, 2H), 3.98 (t, J=6.6 Hz, 2H), 3.79-3.36 (m, 13H), 2.23 (s, 3H), 1.58 (dd, J=12.8, 6.4 Hz, 2H), 0.89 (t, J=7.3 Hz, 3H).

LCMS (ESI-TOF) m/z 484.1 [M+H] with a purity of >95%.

### Propyl 4-(4-chloro-2-(1-(methoxycarbonyl)-1H-pyrrol-3-yl)quinoline-7-carbonyl)piperazine-1-carboxylate (B228)

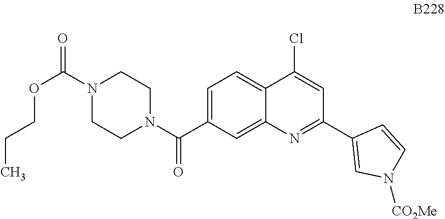 [SMILES: CCCOC(=O)N1CCN(C(=O)c2ccc3c(Cl)cc(-c4ccn(C(=O)OC)c4)nc3c2)CC1]

Compound B228 was synthesized according to General Procedure L using propyl 4-(2,4-dichloroquinoline-7-carbonyl)piperazine-1-carboxylate and 1-(methoxycarbonyl)pyrrole-3-boronic acid pinacol ester as starting materials.

¹H NMR (400 MHz, DMSO-d₆) δ 8.41 (s, 1H), 8.34 (s, 1H), 8.22 (d, J=8.6 Hz, 1H), 8.03 (s, 1H), 7.68 (dd, J=8.6, 1.3 Hz, 1H), 7.49-7.43 (m, 1H), 7.12-7.05 (m, 1H), 4.08-3.94 (m, 5H), 3.73-3.37 (m, 8H), 1.59 (dd, J=14.2, 6.8 Hz, 2H), 0.89 (t, J=7.2 Hz, 3H).

LCMS (ESI-TOF) m/z 485.1 [M+H⁺] with a purity of >99%.

### Propyl 4-(4-chloro-2-(1-isopropyl-1H-pyrazol-4-yl)quinoline-7-carbonyl)piperazine-1-carboxylate (B229)

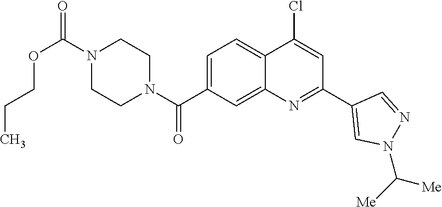 [SMILES: CCCOC(=O)N1CCN(C(=O)c2ccc3c(Cl)cc(-c4cnn(C(C)C)c4)nc3c2)CC1]

Compound B229 was synthesized according to General Procedure L using propyl 4-(2,4-dichloroquinoline-7-carbonyl)piperazine-1-carboxylate and 1-isopropylpyrazole-4-boronic acid pinacol ester as starting materials.

¹H NMR (400 MHz, DMSO-d₆) δ 8.63 (s, 1H), 8.23 (d, J=3.7 Hz, 2H), 8.20 (d, J=8.5 Hz, 1H), 7.98 (s, 1H), 7.65 (dd, J=8.5, 1.4 Hz, 1H), 4.58 (dt, J=13.3, 6.6 Hz, 1H), 3.98 (t, J=6.6 Hz, 2H), 3.82-3.35 (m, 8H), 1.59 (dd, J=13.8, 6.8 Hz, 2H), 1.49 (d, J=6.7 Hz, 6H), 0.89 (t, J=7.2 Hz, 3H).

LCMS (ESI-TOF) m/z 470.1 [M+H⁺] with a purity of >99%.

### Propyl 4-(4-chloro-2-(1-(difluoromethyl)-1H-pyrazol-4-yl)quinoline-7-carbonyl)piperazine-1-carboxylate (B230)

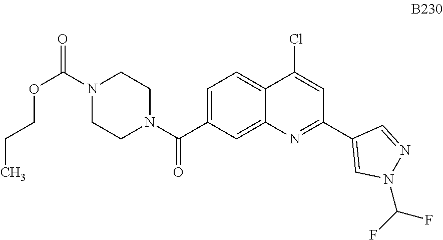 [SMILES: CCCOC(=O)N1CCN(C(=O)c2ccc3c(Cl)cc(-c4cnn(C(F)F)c4)nc3c2)CC1]

Compound B230 was synthesized according to General Procedure L using propyl 4-(2,4-dichloroquinoline-7-carbonyl)piperazine-1-carboxylate and 1-(difluoromethyl)pyrazole-4-boronic acid pinacol ester as starting materials.

¹H NMR (400 MHz, DMSO-d₆) δ 9.12 (s, 1H), 8.57 (s, 1H), 8.38 (s, 1H), 8.25 (d, J=8.5 Hz, 1H), 8.05 (s, 1H), 7.93 (t, J=59.0 Hz, 1H), 7.72 (dd, J=8.5, 1.3 Hz, 1H), 3.98 (t, J=6.6 Hz, 2H), 3.77-3.36 (m, 8H), 1.59 (dd, J=13.9, 6.9 Hz, 2H), 0.89 (t, J=7.2 Hz, 3H).

LCMS (ESI-TOF) m/z 478.1 [M+H⁺] with a purity of >95%.

### Propyl 4-(4-chloro-2-(1-(N,N-dimethylsulfamoyl)-1H-pyrrol-3-yl)quinoline-7-carbonyl)piperazine-1-carboxylate (B231)

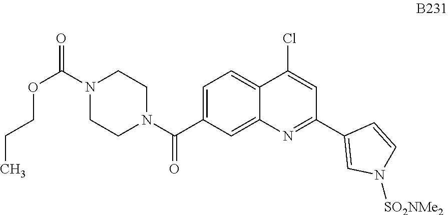

Compound B231 was synthesized according to General Procedure L using propyl 4-(2,4-dichloroquinoline-7-carbonyl)piperazine-1-carboxylate and 1-(N,N-dimethylsulfamoyl)pyrrole-3-boronic acid pinacol ester as starting materials.

¹H NMR (400 MHz, DMSO-d₆) δ 8.43 (s, 1H), 8.24 (s, 1H), 8.22 (d, J=8.5 Hz, 1H), 8.04 (s, 1H), 7.68 (dd, J=8.5, 1.3 Hz, 1H), 7.43-7.31 (m, 1H), 7.15 (dd, J=3.0, 1.4 Hz, 1H), 3.98 (t, J=6.6 Hz, 2H), 3.75-3.38 (m, 8H), 2.84 (s, 6H), 1.59 (dd, J=14.1, 6.9 Hz, 2H), 0.89 (t, J=7.3 Hz, 3H).

LCMS (ESI-TOF) m/z 534.1 [M+H⁺] with a purity of >98%.

### Propyl 4-(4-chloro-2-(1-oxoisoindolin-5-yl)quinoline-7-carbonyl)piperazine-1-carboxylate (B232)

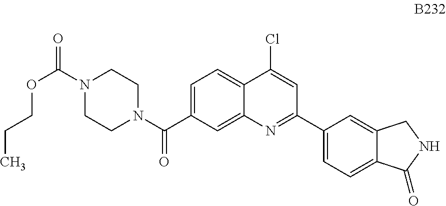 [SMILES: CCCOC(=O)N1CCN(C(=O)c2ccc3c(Cl)cc(-c4ccc5c(c4)CNC5=O)nc3c2)CC1]

Compound B232 was synthesized according to General Procedure L using propyl 4-(2,4-dichloroquinoline-7-carbonyl)piperazine-1-carboxylate and isoindolin-1-one-5-boronic acid pinacol ester as starting materials.

¹H NMR (400 MHz, DMSO-d₆) δ 8.70 (s, 1H), 8.57 (s, 1H), 8.53 (s, 1H), 8.44 (d, J=8.1 Hz, 1H), 8.31 (d, J=8.5 Hz, 1H), 8.18 (s, 1H), 7.85 (d, J=8.0 Hz, 1H), 7.79 (d, J=8.6 Hz, 1H), 4.51 (s, 2H), 3.98 (t, J=6.6 Hz, 2H), 3.81-3.34 (m, 8H), 1.59 (dd, J=14.1, 7.0 Hz, 2H), 0.89 (t, J=7.4 Hz, 3H).

LCMS (ESI-TOF) m/z 493.1 [M+H⁺] with a purity of >99%.

### 3-Fluoropropyl (R)-4-(4-chloro-2-(4-(methylcarbamoyl)phenyl)quinoline-7-carbonyl)-2-methylpiperazine-1-carboxylate (B233)

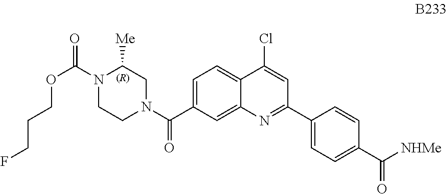

Compound B228 was synthesized according to General Procedure L using 3-fluoropropyl (R)-4-(2,4-dichloroquinoline-7-carbonyl)-2-methylpiperazine-1-carboxylate and 4-(methylcarbamoyl)phenylboronic acid as starting materials.

¹H NMR (400 MHz, 80° C., DMSO-d₆) δ 8.45 (s, 1H), 8.41-8.32 (m, 3H), 8.30 (d, J=8.5 Hz, 1H), 8.14 (s, 1H), 8.00 (d, J=8.4 Hz, 2H), 7.75 (dd, J=8.5, 1.2 Hz, 1H), 4.57 (t, J=5.9 Hz, 1H), 4.45 (t, J=6.0 Hz, 1H), 4.36-3.74 (m, 6H), 3.38-3.11 (m, 3H), 2.84 (d, J=4.5 Hz, 3H), 2.04-1.90 (m, 2H), 1.14 (d, J=6.2 Hz, 3H).

LCMS (ESI-TOF) m/z 527.1 [M+H⁺] with a purity of >95%.

### 3-Fluoropropyl (R)-4-(4-chloro-2-(1-methyl-1H-pyrrol-3-yl)quinoline-7-carbonyl)-2-methylpiperazine-1-carboxylate (B234)

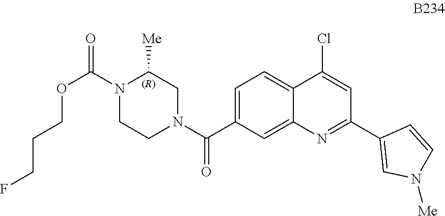

Compound B234 was synthesized according to General Procedure L using 3-fluoropropyl (R)-4-(2,4-dichloroquinoline-7-carbonyl)-2-methylpiperazine-1-carboxylate and N-methylpyrrole-3-boronic acid pinacol ester as starting materials.

¹H NMR (400 MHz, 80° C., DMSO-d₆) δ 8.14 (d, J=8.5 Hz, 1H), 7.98 (s, 1H), 7.88 (s, 1H), 7.64 (s, 1H), 7.55 (d, J=7.2 Hz, 1H), 6.83-6.74 (m, 2H), 4.57 (t, J=5.9 Hz, 1H), 4.45 (t, J=6.0 Hz, 1H), 4.35-3.64 (m, 9H), 3.34-3.09 (m, 3H), 2.06-1.87 (m, 2H), 1.13 (d, J=6.3 Hz, 3H).

LCMS (ESI-TOF) m/z 473.1 [M+H⁺] with a purity of >95%.

### 3-Fluoropropyl (R)-4-(4-chloro-2-(3,5-difluoro-4-hydroxyphenyl)quinoline-7-carbonyl)-2-methylpiperazine-1-carboxylate (B235)

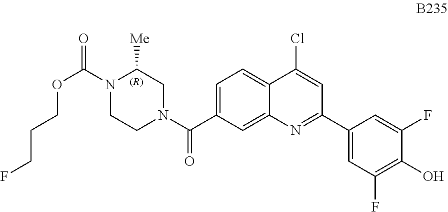

Compound B235 was synthesized according to General Procedure L using 3-fluoropropyl (R)-4-(2,4-dichloroquinoline-7-carbonyl)-2-methylpiperazine-1-carboxylate and 3,5-difluoro-4-hydroxyphenylboronic acid as starting materials.

¹H NMR (400 MHz, 80° C., DMSO-d₆) δ 10.48 (br s, 1H), 8.39 (s, 1H), 8.25 (d, J=8.5 Hz, 1H), 8.11-7.94 (m, 3H), 7.70 (d, J=8.5 Hz, 1H), 4.57 (t, J=5.9 Hz, 1H), 4.45 (t, J=5.9 Hz, 1H), 4.35-3.57 (m, 6H), 3.42-3.09 (m, 3H), 2.08-1.90 (m, 2H), 1.14 (d, J=6.2 Hz, 3H).

LCMS (ESI-TOF) m/z 522.1 [M+H⁺] with a purity of >95%.

### Propyl 4-(4-chloro-2-(3-methyl-4-(methylcarbamoyl)phenyl)quinoline-7-carbonyl)piperazine-1-carboxylate (B236)

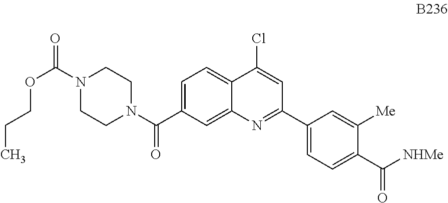 [SMILES: CCCOC(=O)N1CCN(C(=O)c2ccc3c(Cl)cc(-c4ccc(C(=O)NC)c(C)c4)nc3c2)CC1]

Compound B236 was synthesized according to General Procedure L using propyl 4-(2,4-dichloroquinoline-7-carbonyl)piperazine-1-carboxylate and [3-methyl-4-(methylcarbamoyl)phenyl]boronic acid pinacol ester as starting materials.

¹H NMR (400 MHz, DMSO-d₆) δ 8.53 (s, 1H), 8.35-8.14 (m, 5H), 7.76 (d, J=8.5 Hz, 1H), 7.50 (d, J=7.9 Hz, 1H), 3.98 (t, J=6.5 Hz, 2H), 3.78-3.37 (m, 8H), 2.79 (d, J=4.6 Hz, 3H), 1.59 (d, J=6.4 Hz, 2H), 0.89 (t, J=7.2 Hz, 3H).

LCMS (ESI-TOF) m/z 509.1 [M+H⁺] with a purity of >95%.

### Propyl 4-(4-chloro-2-(2-methylbenzo[d]oxazol-5-yl)quinoline-7-carbonyl)piperazine-1-carboxylate (B237)

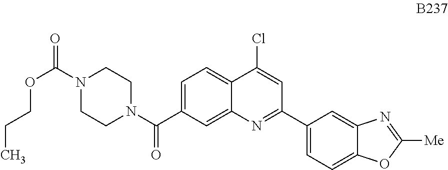 [SMILES: CCCOC(=O)N1CCN(C(=O)c2ccc3c(Cl)cc(-c4ccc5oc(C)nc5c4)nc3c2)CC1]

Compound B237 was synthesized according to General Procedure L using propyl 4-(2,4-dichloroquinoline-7-carbonyl)piperazine-1-carboxylate and 2-methyl-1,3-benzoxazol-5-ylboronic acid as starting materials.

¹H NMR (400 MHz, DMSO-d₆) δ 62 (d, J=7.1 Hz, 1H), 8.60 (s, 1H), 8.39 (dd, J=8.6, 1.6 Hz, 1H), 8.29 (d, J=8.5 Hz, 1H), 8.17 (s, 1H), 7.84 (d, J=8.6 Hz, 1H), 7.78-7.71 (m, 1H), 3.98 (t, J=6.6 Hz, 2H), 3.78-3.38 (m, 8H), 2.67 (s, 3H), 1.59 (dd, J=13.6, 6.5 Hz, 2H), 0.89 (t, J=7.1 Hz, 4H).

LCMS (ESI-TOF) m/z 493.1 [M+H⁺] with a purity of >96%.

### Propyl 4-(4-chloro-2-(3-fluoro-4-(pyrrolidin-1-yl)phenyl)quinoline-7-carbonyl)piperazine-1-carboxylate (B238)

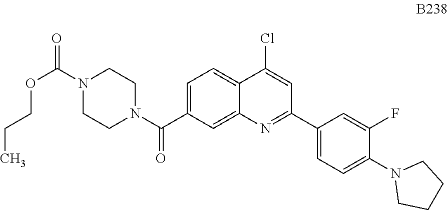 [SMILES: CCCOC(=O)N1CCN(C(=O)c2ccc3c(Cl)cc(-c4ccc(N5CCCC5)c(F)c4)nc3c2)CC1]

Compound B238 was synthesized according to General Procedure L using propyl 4-(2,4-dichloroquinoline-7-carbonyl)piperazine-1-carboxylate and 3-fluoro-4-pyrrolidinylphenylboronic acid as starting materials.

¹H NMR (400 MHz, 80° C., DMSO-d₆) δ 8.34 (s, 1H), 8.25 (d, J=8.6 Hz, 1H), 8.10 (d, J=1.0 Hz, 1H), 7.71 (dd, J=8.5, 1.5 Hz, 1H), 7.64 (dd, J=9.1, 2.2 Hz, 1H), 7.60-7.53 (m, 1H), 7.19 (dd, J=13.8, 8.3 Hz, 1H), 3.99 (t, J=6.5 Hz, 2H), 3.68-3.37 (m, 12H), 1.96 (t, J=6.5 Hz, 4H), 1.60 (dd, J =14.2, 7.0 Hz, 2H), 0.89 (t, J=7.4 Hz, 3H).

LCMS (ESI-TOF) m/z 525.1 +111 with a purity of >99%.

Propyl 4-(4-chloro-2-(4-(1-ethoxyethyl)phenyl)quinoline-7-carbonyl)piperazine-1-carboxylate

(B239)

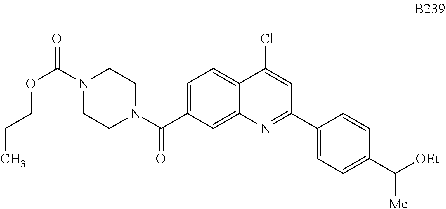 [SMILES: CCCOC(=O)N1CCN(C(=O)c2ccc3c(Cl)cc(-c4ccc(C(C)OCC)cc4)nc3c2)CC1]

Compound B239 was synthesized according to General Procedure L using propyl 4-(2,4-dichloroquinoline-7-carbonyl)piperazine-1-carboxylate and 4-(1-ethoxyethyl)phenylboronic acid as starting materials.

¹H NMR (400 MHz, 80° C., DMSO-d₆) 6 8.36 (s, 1H), 8.26 (dd, J=8.3, 4.3 Hz, 3H), 8.12 (d, J=1.1 Hz, 1H), 7.72 (d, J=7.0 Hz, 1H), 7.50 (d, J=8.1 Hz, 2H), 4.54 (q, J=6.4 Hz, 1H), 3.99 (t, J=6.6 Hz, 2H), 3.69-3.29 (m, 10H), 1.60 (dd, J=14.0, 6.8 Hz, 2H), 1.40 (d, J=6.4 Hz, 3H), 1.13 (t, J=7.0 Hz, 3H), 0.90 (t, J=7.4 Hz, 3H).

LCMS (ESI-TOF) m/z 510.1 [M+H⁺] with a purity of >94%.

### Propyl (S)-4-(4-chloro-2-(3-fluoro-4-(methylcarbamoyl)phenyl)quinoline-7-carbonyl)-3-methylpiperazine-1-carboxylate (B240)

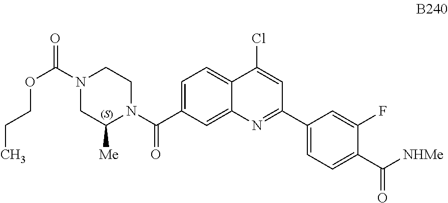

Compound B240 was synthesized according to General Procedure L using propyl (S)-4-(2,4-dichloroquinoline-7-carbonyl)-3-methylpiperazine-1-carboxylate and 3-fluoro-4-(methylcarbamoyl)phenylboronic acid as starting materials.

¹H NMR (400 MHz, DMSO-d₆) δ 8.60 (s, 1H), 8.40-8.34 (m, 1H), 8.31 (d, J=8.5 Hz, 1H), 8.29-8.23 (m, 2H), 8.16 (d, J=1.0 Hz, 1H), 7.81 (t, J=7.8 Hz, 1H), 7.77 (dd, J=8.6, 1.5 Hz, 1H), 4.37-2.88 (m, 9H), 2.82 (d, J=4.6 Hz, 3H), 1.58 (dd, J=14.1, 6.7 Hz, 2H), 1.19 (s, 3H), 0.89 (t, J=7.3 Hz, 3H).

LCMS (ESI-TOF) m/z 527.1 [M+H⁺] with a purity of >97%.

### Propyl (S)-4-(2-(benzo[d]oxazol-5-yl)-4-chloroquinoline-7-carbonyl)-3-methylpiperazine-1-carboxylate (B241)

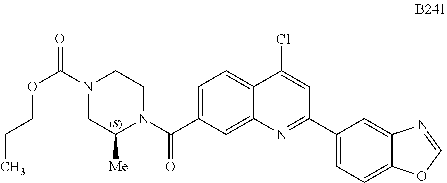

Compound B241 was synthesized according to General Procedure L using propyl (S)-4-(2,4-dichloroquinoline-7-carbonyl)-3-methylpiperazine-1-carboxylate and 1,3-benzoxazole-5-boronic acid pinacol ester as starting materials.

¹H NMR (400 MHz, DMSO-d₆) δ 8.86 (s, 1H), 8.78 (d, J=1.3 Hz, 1H), 8.63 (s, 1H), 8.49 (dd, J=8.7, 1.4 Hz, 1H), 8.30 (d, J=8.6 Hz, 1H), 8.15 (s, 1H), 7.97 (d, J=8.7 Hz, 1H), 7.74 (d, J=9.8 Hz, 1H), 4.32-3.71 (m, 6H), 3.25-2.83 (m, 3H), 1.59 (d, J=7.1 Hz, 2H), 1.19 (d, J=2.4 Hz, 3H), 0.89 (t, J=7.2 Hz, 3H).

LCMS (ESI-TOF) m/z 493.1 [M+H⁺] with a purity of >98%.

### Propyl 4-(4-chloro-2-(4-((cyclopropylmethoxy)methyl)phenyl)quinoline-7-carbonyl)piperazine-1-carboxylate (B242)

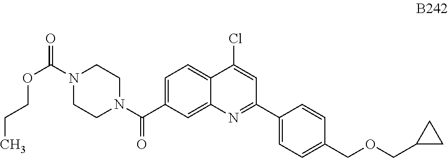 [SMILES: CCCOC(=O)N1CCN(C(=O)c2ccc3c(Cl)cc(-c4ccc(COCC5CC5)cc4)nc3c2)CC1]

Compound B242 was synthesized according to General Procedure L using propyl 4-(2,4-dichloroquinoline-7-carbonyl)piperazine-1-carboxylate and 4-[(cyclopropylmethoxy)methyl]phenylboronic acid as starting materials.

¹H NMR (400 MHz, DMSO-d₆) δ 8.48 (s, 1H), 8.32 (d, J=8.3 Hz, 2H), 8.28 (d, J=8.5 Hz, 1H), 8.14 (d, J=0.9 Hz, 1H), 7.75 (dd, J=8.5, 1.4 Hz, 1H), 7.52 (d, J=8.2 Hz, 2H), 4.58 (s, 2H), 3.98 (t, J=6.6 Hz, 2H), 3.78-3.38 (m, 8H), 3.34 (d, J=6.8 Hz, 2H), 1.59 (dd, J=13.8, 6.8 Hz, 2H), 1.15-1.01 (m, 1H), 0.89 (t, J=7.2 Hz, 3H), 0.54-0.45 (m, 2H), 0.24-0.14 (m, 2H).

LCMS (ESI-TOF) m/z 522.2 [M+H⁺] with a purity of >96%.

### Propyl (R)-4-(2-(benzo[d]oxazol-5-yl)-4-chloroquinoline-7-carbonyl)-2-methylpiperazine-1-carboxylate (B243)

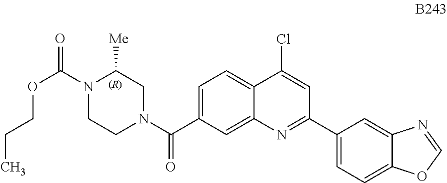

Compound B243 was synthesized according to General Procedure L using propyl (R)-4-(2,4-dichloroquinoline-7-carbonyl)-2-methylpiperazine-1-carboxylate and 1,3-benzoxazole-5-boronic acid pinacol ester as starting materials.

¹H NMR (400 MHz, 80° C., DMSO-d₆) δ 8.76 (s, 1H), 8.72 (s, 1H), 8.52 (s, 1H), 8.44 (dd, J=8.6, 1.5 Hz, 1H), 8.29 (d, J=8.5 Hz, 1H), 8.14 (s, 1H), 7.91 (d, J=8.7 Hz, 1H), 7.73 (dd, J=8.5, 1.3 Hz, 1H), 4.41-3.59 (m,6H), 3.22 (dd, J=35.0, 20.1 Hz, 3H), 1.66-1.53 (m, 2H), 1.15 (d, J=6.2 Hz, 3H), 0.89 (t, J=7.4 Hz, 3H).

LCMS (ESI-TOF) m/z 493.1 [M+H⁺] with a purity of >96%.

### Propyl 4-(4-chloro-2-(4-(2-hydroxypropan-2-yl)phenyl)quinoline-7-carbonyl)piperazine-1-carboxylate (B244)

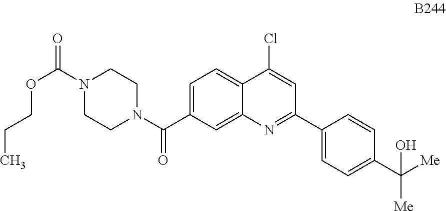 [SMILES: CCCOC(=O)N1CCN(C(=O)c2ccc3c(Cl)cc(-c4ccc(C(C)(C)O)cc4)nc3c2)CC1]

Compound B244 was synthesized according to General Procedure L using propyl 4-(2,4-dichloroquinoline-7-carbonyl)piperazine-1-carboxylate and 4-(2-hydroxy-2-propanyl)phenylboronic acid as starting materials.

¹H NMR (400 MHz, 80° C., DMSO-d₆) 6 8.46 (s, 1H), 8.30-8.22 (m, 3H), 8.13 (s, 1H), 7.74 (d, J=8.5 Hz, 1H), 7.66 (d, J=8.4 Hz, 2H), 5.15 (s, 1H), 3.98 (t, J=6.6 Hz, 2H), 3.79-3.39 (m, 8H), 1.59 (dd, J=13.7, 6.8 Hz, 2H), 1.48 (s, 6H), 0.89 (t, J=7.2 Hz, 3H).

LCMS (ESI-TOF) m/z 496.1 [M+H⁺] with a purity of >98%.

### Isobutyl 4-(4-chloro-2-(4-(methylcarbamoyl)phenyl)quinoline-7-carbonyl)piperazine-1-carboxylate (B245)

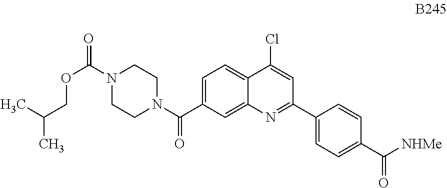 [SMILES: CNC(=O)c1ccc(-c2cc(Cl)c3ccc(C(=O)N4CCN(C(=O)OCC(C)C)CC4)cc3n2)cc1]

Compound B245 was synthesized according to General Procedure L using isobutyl 4-(2,4-dichloroquinoline-7-carbonyl)piperazine-1-carboxylate and 4-(N-methylcarbamoyl)phenylboronic acid as starting materials.

¹H NMR (400 MHz, DMSO-d₆) 6 8.63-8.57 (m, 1H), 8.57 (s, 1H), 8.42 (d, J=8.4 Hz, 2H), 8.30 (d, J=8.6 Hz, 1H), 8.18 (s, 1H), 8.02 (d, J=8.4 Hz, 2H), 7.78 (dd, J=8.5, 1.1 Hz, 1H), 3.82 (d, J=6.5 Hz, 2H), 3.77-3.40 (m, 8H), 2.83 (d, J=4.5 Hz, 3H), 1.87 (s, 1H), 0.89 (d, J=6.4 Hz, 6H).

LCMS (ESI-TOF) m/z 509.1 [M+H⁺] with a purity of >98%.

### Isobutyl 4-(4-chloro-2-(1-methyl-1H-pyrrol-3-yl)quinoline-7-carbonyl)piperazine-1-carboxylate (B246)

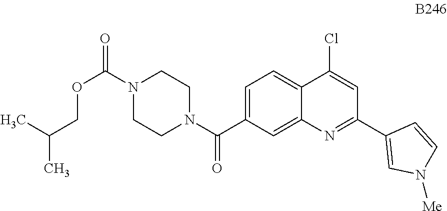 [SMILES: CC(C)COC(=O)N1CCN(C(=O)c2ccc3c(Cl)cc(-c4ccn(C)c4)nc3c2)CC1]

Compound B246 was synthesized according to General Procedure L using isobutyl 4-(2,4-dichloroquinoline-7-carbonyl)piperazine-1-carboxylate and 1-methylpyrrole-3-boronic acid pinacol ester as starting materials.

¹H NMR (400 MHz, DMSO-d₆) δ 8.14 (d, J=8.5 Hz, 1H), 8.07 (s, 1H), 7.91 (s, 1H), 7.70 (s, 1H), 7.61-7.53 (m, 1H), 6.88-6.77 (m, 2H), 3.81 (d, J=6.5 Hz, 2H), 3.71 (s, 3H), 3.69-3.34 (m, 8H), 1.96-1.75 (m, 1H), 0.89 (d, J=6.5 Hz, 6H).

LCMS (ESI-TOF) m/z 455.1 [M+H⁺] with a purity of >98%.

### 2-Fluoroethyl 4-(4-chloro-2-(4-(methylcarbamoyl)phenyl)quinoline-7-carbonyl)piperazine-1-carboxylate (B247)

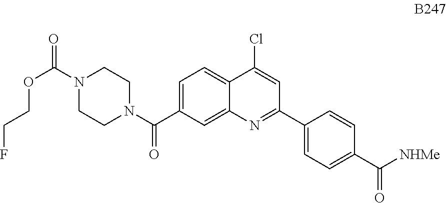 [SMILES: CNC(=O)c1ccc(-c2cc(Cl)c3ccc(C(=O)N4CCN(C(=O)OCCF)CC4)cc3n2)cc1]

Compound B247 was synthesized according to General Procedure L using 2-fluoroethyl 4-(2,4-dichloroquinoline-7-carbonyl)piperazine-1-carboxylate and 4-(N-methylcarbamoyl)phenylboronic acid as starting materials.

¹H NMR (400 MHz, DMSO-d₆) δ 8.61-8.57 (m, 1H), 8.57 (s, 1H), 8.42 (d, J=8.4 Hz, 2H), 8.30 (d, J=8.5 Hz, 1H), 8.18 (s, 1H), 8.02 (d, J=8.4 Hz, 2H), 7.78 (dd, J=8.5, 1.3 Hz, 1H), 4.61 (d, J=47.6 Hz, 2H), 4.37-4.20 (m, 2H), 3.81-3.40 (m, 8H), 2.83 (d, J=4.5 Hz, 3H).

LCMS (ESI-TOF) m/z 499.1 [M+H⁺] with a purity of >97%.

### 2-Fluoroethyl 4-(4-chloro-2-(1-methyl-1H-pyrrol-3-yl)quinoline-7-carbonyl)piperazine-1-carboxylate (B248)

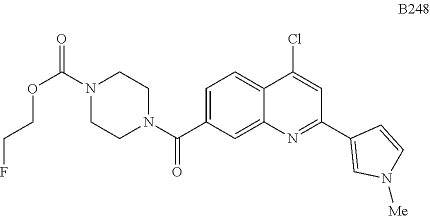 [SMILES: Cn1ccc(-c2cc(Cl)c3ccc(C(=O)N4CCN(C(=O)OCCF)CC4)cc3n2)c1]

Compound B248 was synthesized according to General Procedure L using 2-fluoroethyl 4-(2,4-dichloroquinoline-7-carbonyl)piperazine-1-carboxylate and 1-methylpyrrole-3-boronic acid as starting materials.

¹H NMR (400 MHz, DMSO-d₆) δ 8.15 (d, J=8.5 Hz, 1H), 8.07 (s, 1H), 7.92 (s, 1H), 7.71 (s, 1H), 7.58 (d, J=8.5 Hz, 1H), 6.83 (d, J=16.7 Hz, 2H), 4.61 (d, J=46.7 Hz, 2H), 4.45-4.16 (m, 2H), 3.71 (s, 5H), 3.43 (s, 7H).

LCMS (ESI-TOF) m/z 445.1 [M+⁺] with a purity of >98%.

### Propyl 4-(2-(1-(2-amino-2-oxoethyl)-1H-pyrazol-4-yl)-4-chloroquinoline-7-carbonyl)piperazine-1-carboxylate (B249)

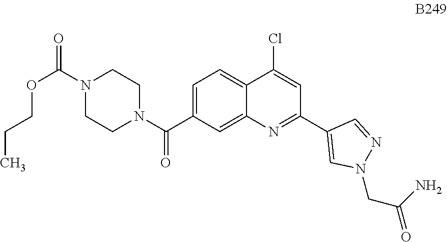 [SMILES: CCCOC(=O)N1CCN(C(=O)c2ccc3c(Cl)cc(-c4cnn(CC(N)=O)c4)nc3c2)CC1]

Compound B249 was synthesized according to General Procedure L using propyl 4-(2,4-dichloroquinoline-7-carbonyl)piperazine-1-carboxylate and 1-(2-amino-2-oxoethyl)-1H-pyrazol-4-ylboronic acid pinacol ester as starting materials.

¹H NMR (400 MHz, DMSO-d₆) δ 8.54 (s, 1H), 8.26 (s, 1H), 8.23 (s, 1H), 8.21 (d, J=8.5 Hz, 1H), 7.99 (s, 1H), 7.70-7.62 (m, 1H), 7.59 (s, 1H), 7.32 (s, 1H), 4.87 (s, 2H), 3.98 (t, J=6.6 Hz, 2H), 3.77-3.33 (m, 8H), 1.59 (dd, J=14.0, 7.0 Hz, 2H), 0.89 (t, J=7.3 Hz, 3H).

LCMS (ESI-TOF) m/z 485.1 [M+H⁺] with a purity of >99%.

### Propyl 4-(4-chloro-2-(5-(trifluoromethyl)-1H-pyrazol-4-yl)quinoline-7-carbonyl)piperazine-1-carboxylate (B250)

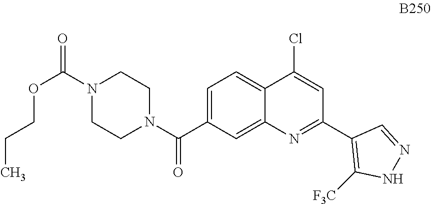 [SMILES: CCCOC(=O)N1CCN(C(=O)c2ccc3c(Cl)cc(-c4cn[nH]c4C(F)(F)F)nc3c2)CC1]

Compound B250 was synthesized according to General Procedure L using propyl 4-(2,4-dichloroquinoline-7-carbonyl)piperazine-1-carboxylate and 5-trifluoromethyl-1H-pyrazol-4-ylboronic acid as starting materials.

¹H NMR (400 MHz, DMSO-d₆) δ 13.99 (br s, 1H), 8.82 (s, 1H), 8.25 (d, J=8.5 Hz, 1H), 8.20 (s, 1H), 7.97 (s, 1H), 7.73 (d, J=8.7 Hz, 1H), 3.98 (t, J=6.5 Hz, 2H), 3.81-3.34 (m, 8H), 1.58 (dd, J=12.0, 5.6 Hz, 2H), 0.89 (t, J=6.9 Hz, 3H).

LCMS (ESI-TOF) m/z 496.1 [M+H⁺] with a purity of >98%.

### Propyl (R)-4-(4-chloro-2-(1H-pyrrol-3-yl)quinoline-7-carbonyl)-2-methylpiperazine-1-carboxylate (B251)

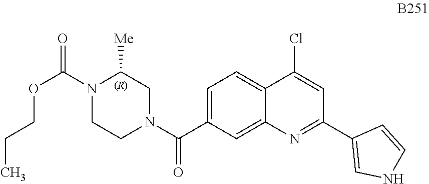

Compound B251 was synthesized according to General Procedure L using propyl (R)-4-(2,4-dichloroquinoline-7-carbonyl)-2-methylpiperazine-1-carboxylate and 1H-pyrrole-3-boronic acid pinacol ester as starting materials.

¹H NMR (400 MHz, 80° C., DMSO-d₆) δ 11.10 (s, 1H), 8.14 (d, J=8.4 Hz, 1H), 8.03 (s, 1H), 7.90 (s, 1H), 7.68 (s, 1H), 7.55 (dd, J=8.5, 1.5 Hz, 1H), 6.84 (d, J=13.5 Hz, 2H), 4.41-3.69 (m, 6H), 3.32-3.11 (m, 3H), 1.67-1.47 (m, 2H), 1.13 (d, J=6.4 Hz, 3H), 0.89 (t, J=7.4 Hz, 3H).

LCMS (ESI-TOF) m/z 441.0 [M+H⁺] with a purity of >98%.

### Propyl 4-(4-chloro-2-(1-(methylsulfonyl)-1H-pyrrol-3-yl)quinoline-7-carbonyl)piperazine-1-carboxylate (B252)

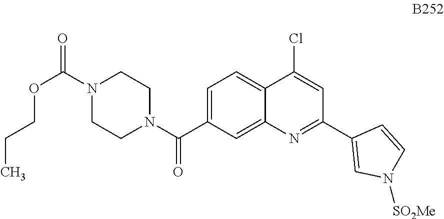 [SMILES: CCCOC(=O)N1CCN(C(=O)c2ccc3c(Cl)cc(-c4ccn(S(C)(=O)=O)c4)nc3c2)CC1]

Compound B252 was synthesized according to General Procedure L using propyl 4-(2,4-dichloroquinoline-7-carbonyl)piperazine-1-carboxylate and 1-(methylsulfonyl)pyrrole-3-boronic acid pinacol ester as starting materials.

¹H NMR (400 MHz, 80° C., DMSO-d₆) δ 8.26 (s, 1H), 8.21 (t, J=8.8 Hz, 1H), 8.17 (t, J=1.8 Hz, 1H), 8.02 (d, J=1.1 Hz, 1H), 7.67 (dd, J=8.5, 1.5 Hz, 1H), 7.40-7.35 (m, 1H), 7.14 (dd, J=3.2, 1.6 Hz, 1H), 3.99 (t, J=6.6 Hz, 2H), 3.50 (d, J=26.1 Hz, 11H), 1.65-1.52 (m, 2H), 0.90 (t, J=7.4 Hz, 3H).

LCMS (ESI-TOF) m/z 505.1 [M+H⁺] with a purity of >99%.

### Propyl 4-(2-(4-carbamoyl-3,5-difluorophenyl)-4-chloroquinoline-7-carbonyl)piperazine-1-carboxylate (B253)

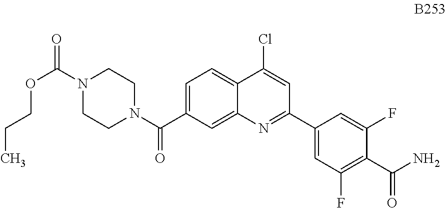 [SMILES: CCCOC(=O)N1CCN(C(=O)c2ccc3c(Cl)cc(-c4cc(F)c(C(N)=O)c(F)c4)nc3c2)CC1]

Compound B253 was synthesized according to General Procedure L using propyl 4-(2,4-dichloroquinoline-7-carbonyl)piperazine-1-carboxylate and 3,5-difluoro-4-(carbamoyl)phenylboronic acid as starting materials.

¹H NMR (400 MHz, 80° C., DMSO-d₆) δ 8.52 (s, 1H), 8.30 (d, J=8.6 Hz, 1H), 8.18 (s, 1H), 8.07 (d, J=8.8 Hz, 2H), 7.98 (s, 1H), 7.78 (dd, J=8.5, 1.3 Hz, 1H), 7.65 (s, 1H), 4.00 (t, J=6.5 Hz, 2H), 3.66-3.39 (m, 8H), 1.65-1.51 (m, 2H), 0.90 (t, J=7.4 Hz, 3H).

LCMS (ESI-TOF) m/z 517.1 [M+H⁺] with a purity of >96%.

### Propyl (S)-4-(4-chloro-2-(1H-pyrrol-3-yl)quinoline-7-carbonyl)-3-methylpiperazine-1-carboxylate (B254)

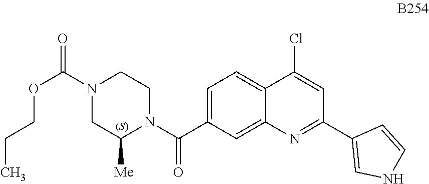

Compound B254 was synthesized according to General Procedure L using propyl (S)-4-(2,4-dichloroquinoline-7-carbonyl)-3-methylpiperazine-1-carboxylate and 1H-pyrrole-3-boronic acid pinacol ester as starting materials.

¹H NMR (400 MHz, 80° C., DMSO-d₆) δ 11.14 (br s, 1H), 8.14 (d, J=8.4 Hz, 1H), 8.04 (s, 1H), 7.88 (s, 1H), 7.69 (s, 1H), 7.53 (dd, J=8.5, 1.5 Hz, 1H), 6.89-6.80 (m, 2H), 4.47-3.73 (m, 6H), 3.35-2.88 (m, 3H), 1.65-1.53 (m, 2H), 1.19 (d, J=6.7 Hz, 3H), 0.89 (t, J=7.4 Hz, 3H).

LCMS (ESI-TOF) m/z 441.1 [M+H⁺] with a purity of >98%.

### Propyl 4-(4-chloro-2-(4-(hydroxymethyl)phenyl)quinoline-7-carbonyl)piperazine-1-carboxylate (B255)

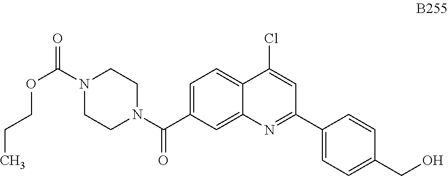 [SMILES: CCCOC(=O)N1CCN(C(=O)c2ccc3c(Cl)cc(-c4ccc(CO)cc4)nc3c2)CC1]

Compound B255 was synthesized according to General Procedure L using propyl 4-(2,4-dichloroquinoline-7-carbonyl)piperazine-1-carboxylate and 4-(hydroxymethyl)phenylboronic acid as starting materials.

¹H NMR (400 MHz, 80° C., DMSO-d₆) δ 8.36 (s, 1H), 8.26 (dd, J=8.4, 5.1 Hz, 3H), 8.12 (d, J=1.1 Hz, 1H), 7.71 (dd, J=8.5, 1.5 Hz, 1H), 7.51 (d, J=8.2 Hz, 2H), 5.05 (br s, 1H), 4.61 (s, 2H), 4.00 (t, J=6.6 Hz, 2H), 3.72-3.39 (m, 8H), 1.70-1.52 (m, 2H), 0.90 (t, J=7.4 Hz, 3H).

LCMS (ESI-TOF) m/z 468.1 [M+H⁺] with a purity of >99%.

Propyl 4-(4-chloro-2-(4-(ethoxymethyl)phenyl)quinoline-7-carbonyl)piperazine-1-carboxylate (B256)

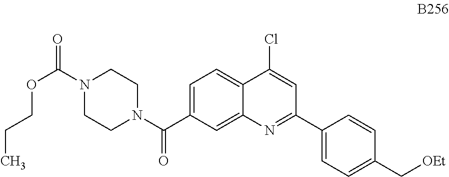 [SMILES: CCCOC(=O)N1CCN(C(=O)c2ccc3c(Cl)cc(-c4ccc(COCC)cc4)nc3c2)CC1]

Compound B256 was synthesized according to General Procedure L using propyl 4-(2,4-dichloroquinoline-7-carbonyl)piperazine-1-carboxylate and 4-(ethoxymethyl)phenylboronic acid as starting materials.

¹H NMR (400 MHz, 80° C., DMSO-d₆) δ 8.36 (s, 1H), 8.27 (d, J=7.9 Hz, 3H), 8.12 (s, 1H), 7.72 (d, J=8.5 Hz, 1H), 7.50 (d, J=8.1 Hz, 2H), 4.56 (s, 2H), 4.00 (t, J=6.5 Hz, 2H), 3.65-3.42 (m, 10H), 1.72-1.51 (m, 2H), 1.20 (t, J=7.0 Hz, 3H), 0.90 (t, J=7.4 Hz, 3H).

LCMS (ESI-TOF) m/z 496.1 [M+H⁺] with a purity of >99%.

### Propyl 4-(4-chloro-2-(3-fluoro-4-(hydroxymethyl)phenyl)quinoline-7-carbonyl)piperazine-1-carboxylate (B257)

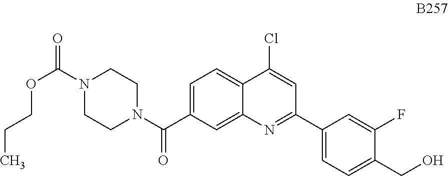 [SMILES: CCCOC(=O)N1CCN(C(=O)c2ccc3c(Cl)cc(-c4ccc(CO)c(F)c4)nc3c2)CC1]

Compound B257 was synthesized according to General Procedure L using propyl 4-(2,4-dichloroquinoline-7-carbonyl)piperazine-1-carboxylate and 3-fluoro-4-(hydroxymethyl)phenylboronic acid as starting materials.

¹H NMR (400 MHz, DMSO-d₆) δ 8.53 (s, 1H), 8.29 (d, J=8.6 Hz, 1H), 8.20 (dd, J=8.0, 1.5 Hz, 1H), 8.16 (d, J=1.1 Hz, 1H), 8.12 (dd, J=11.7, 1.5 Hz, 1H), 7.76 (dd, J=8.5, 1.6 Hz, 1H), 7.67 (t, J=7.9 Hz, 1H), 5.42 (br s, 1H), 4.65 (s, 2H), 3.98 (t, J=6.6 Hz, 2H), 3.78-3.48 (m, 8H), 1.59 (dd, J=14.0, 7.0 Hz, 2H), 0.89 (t, J=7.1 Hz, 3H).

LCMS (ESI-TOF) m/z 486.1 [M+H⁺] with a purity of >99%.

### Propyl 4-(2-(4-(aminomethyl)-3,5-difluorophenyl)-4-chloroquinoline-7-carbonyl)piperazine-1-carboxylate (B258)

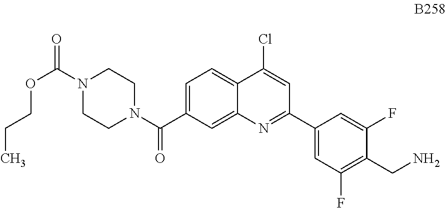 [SMILES: CCCOC(=O)N1CCN(C(=O)c2ccc3c(Cl)cc(-c4cc(F)c(CN)c(F)c4)nc3c2)CC1]

Compound B258 was synthesized according to General Procedure L using propyl 4-(2,4-dichloroquinoline-7-carbonyl)piperazine-1-carboxylate and 3,5-difluoro-4-(aminomethyl)phenylboronic acid as starting materials.

LCMS (ESI-TOF) m/z 503.1 [M+H⁺] with a purity of >94%.

### 2-Fluoroethyl (R)-4-(4-chloro-2-cyclopropylquinoline-7-carbonyl)-2-methylpiperazine-1-carboxylate (B259)

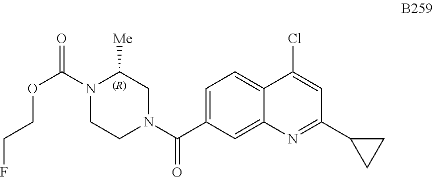

Compound B259 was synthesized according to General Procedure L using 2-fluoroethyl (R)-4-(2,4-dichloroquinoline-7-carbonyl)-2-methylpiperazine-1-carboxylate and cyclopropyl boronic acid as starting materials.

¹H NMR (600 MHz, DMSO-d₆) δ 8.19 (d, J=8.5 Hz, 1H), 7.88 (d, J=29.3 Hz, 1H), 7.80 (s, 1H), 7.63 (d, J=14.7 Hz, 1H), 4.64 (t, J=3.8 Hz, 1H), 4.56 (t, J=3.8 Hz, 1H), 4.46-4.17 (m, 3H), 3.99 (d, J=124.3 Hz, 1H), 3.63 (d, J=101.9 Hz, 1H), 3.50-3.36 (m, 1H), 3.24-2.90 (m, 3H), 2.36-2.26 (m, 1H), 1.26-0.94 (m, 7H).

LCMS (ESI-TOF) m/z 420.1 [M+H⁺] with a purity of >98%.

### 2-Fluoroethyl (S)-4-(4-chloro-2-cyclopropylquinoline-7-carbonyl)-3-methylpiperazine-1-carboxylate (B260)

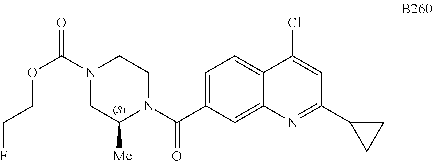

Compound B260 was synthesized according to General Procedure L using 2-fluoroethyl (S)-4-(2,4-dichloroquinoline-7-carbonyl)-3-methylpiperazine-1-carboxylate and cyclopropyl boronic acid as starting materials.

¹H NMR (600 MHz, DMSO-d₆) δ 8.19 (d, J=8.5 Hz, 1H), 7.89 (d, J=1.1 Hz, 1H), 7.81 (s, 1H), 7.63 (dd, J=8.5, 1.4 Hz, 1H), 4.89-3.61 (m, 8H), 3.10 (dd, J=112.3, 62.2 Hz, 3H), 2.38-2.30 (m, 1H), 1.23-1.01 (m, 7H).

LCMS (ESI-TOF) m/z 420.1 [M+H⁺] with a purity of >99%.

### 2-Fluoroethyl 4-(4-chloro-2-(1-methyl-1H-pyrazol-4-yl)quinoline-7-carbonyl)piperazine-1-carboxylate (B261)

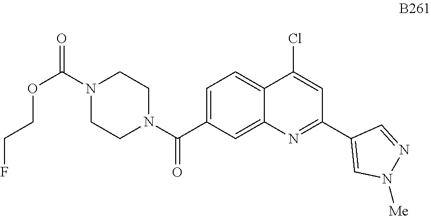 [SMILES: Cn1cc(-c2cc(Cl)c3ccc(C(=O)N4CCN(C(=O)OCCF)CC4)cc3n2)cn1]

Compound B261 was synthesized according to General Procedure L using 2-fluoroethyl 4-(2,4-dichloroquinoline-7-carbonyl)piperazine-1-carboxylate and 1-methyl-1H-pyrazole-4-boronic acid as starting materials.

¹H NMR (400 MHz, DMSO-d₆) δ 8.54 (s, 1H), 8.25-8.16 (m, 3H), 7.98 (d, J=1.1 Hz, 1H), 7.66 (dd, J=8.5, 1.5 Hz, 1H), 4.61 (d, J=48.0 Hz, 2H), 4.37-4.21 (m, 2H), 3.93 (s, 3H), 3.77-3.40 (m, 8H).

LCMS (ESI-TOF) m/z 446.1 [M+H⁺] with a purity of >98%.

### Propyl 4-(4-chloro-3-fluoro-2-(1-methyl-1H-pyrrol-3-yl)quinoline-7-carbonyl)piperazine-1-carboxylate (B262)

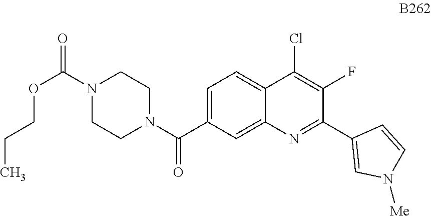 [SMILES: CCCOC(=O)N1CCN(C(=O)c2ccc3c(Cl)c(F)c(-c4ccn(C)c4)nc3c2)CC1]

Propyl 4-(2,4-dichloro-3-fluoroquinoline-7-carbonyl)piperazine-1-carboxylate was synthesized using General Procedure 2 for synthesis of quinolines using 2-fluoro-propanedioic acid instead of malonic acid as starting material. Compound B262 was then synthesized according to General Procedure L using propyl 4-(2,4-dichloro-3-fluoroquinoline-7-carbonyl)piperazine-1-carboxylate and N-methylpyrrole-3-boronic acid pinacol ester as starting materials.

¹H NMR (600 MHz, DMSO-d₆) δ 8.13 (d, J=8.5 Hz, 1H), 7.97 (d, J=1.3 Hz, 1H), 7.69-7.62 (m, 2H), 6.92 (t, J=2.3 Hz, 1H), 6.86 (d, J=1.3 Hz, 1H), 3.98 (t, J=6.6 Hz, 2H), 3.79-3.33 (m, 11H), 1.64-1.51 (m, 2H), 0.89 (t, J=7.4 Hz, 3H).

LCMS (ESI-TOF) m/z 459.1 [M+H⁺] with a purity of >95%.

### Propyl 4-(4-chloro-2-(1-fluorocyclopropyl)quinoline-7-carbonyl)piperazine-1-carboxylate (B263)

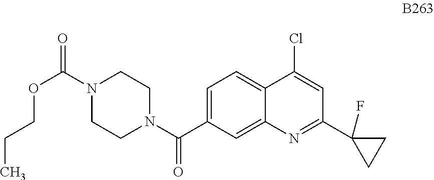 [SMILES: CCCOC(=O)N1CCN(C(=O)c2ccc3c(Cl)cc(C4(F)CC4)nc3c2)CC1]

Compound B263 was synthesized using General Procedure I, K and C using 1-fluorocyclopropyl methyl ketone (General Procedure I) and propyl piperazine-1-carboxylate (General Procedure C) as starting materials.

¹H NMR (400 MHz, DMSO-d₆) δ 8.28 (d, J=8.5 Hz, 1H), 7.98 (s, 2H), 7.74 (dd, J=8.5, 1.4 Hz, 1H), 3.97 (t, J=6.6 Hz, 2H), 3.72-3.45 (m, 8H), 1.75-1.50 (m, 6H), 0.89 (t, J=7.2 Hz, 3H).

LCMS (ESI-TOF) m/z 420.1 [M+H⁺] with a purity of >97%.

### Propyl 4-(2-(4-(azidomethyl)phenyl)-4-chloroquinoline-7-carbonyl)piperazine-1-carboxylate (B264)

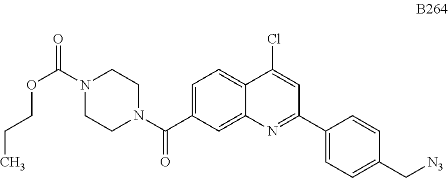 [SMILES: CCCOC(=O)N1CCN(C(=O)c2ccc3c(Cl)cc(-c4ccc(CN=[N+]=[N-])cc4)nc3c2)CC1]

Compound B264 was synthesized according to General Procedure L using propyl 4-(2,4-dichloroquinoline-7-carbonyl)piperazine-1-carboxylate and 4-(azidomethyl) benzeneboronic acid pinacol ester as starting materials.

¹H NMR (400 MHz, DMSO-d₆) δ 8.51 (s, 1H), 8.37 (d, J=8.3 Hz, 2H), 8.29 (d, J=8.5 Hz, 1H), 8.15 (d, J=1.2 Hz, 1H), 7.76 (dd, J=8.5, 1.6 Hz, 1H), 7.58 (d, J=8.3 Hz, 2H), 4.58 (s, 2H), 3.98 (t, J=6.6 Hz, 2H), 3.75-3.34 (m, 8H), 1.59 (dd, J=14.0, 7.1 Hz, 2H), 0.89 (t, J=7.3 Hz, 3H).

LCMS (ESI-TOF) m/z 493.1 [M+H⁺] with a purity of >97%.

### Propyl (R)-4-(4-chloro-2-(4-(2-hydroxypropan-2-yl)phenyl)quinoline-7-carbonyl)-2-methylpiperazine-1-carboxylate (B265)

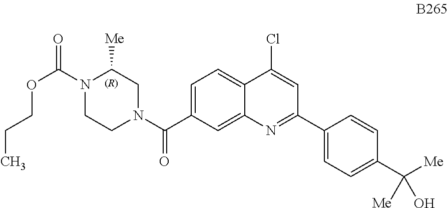

Compound B265 was synthesized according to General Procedure L using propyl (R)-4-(2,4-dichloroquinoline-7-carbonyl)-2-methylpiperazine-1-carboxylate and 4-(2-hydroxypropan-2-yl)phenylboronic acid as starting materials.

¹H NMR (400 MHz, 80° C., DMSO-d₆) δ 8.36 (s, 1H), 8.27 (d, J=8.5 Hz, 1H), 8.22 (d, J=8.4 Hz, 2H), 8.09 (s, 1H), 7.71 (d, J=8.5 Hz, 1H), 7.65 (d, J=8.4 Hz, 2H), 4.88 (s, 1H), 4.30-3.73 (m, 6H), 3.34-3.11 (m, 3H), 1.66-1.54 (m, 2H), 1.50 (s, 6H), 1.14 (d, J=6.3 Hz, 3H), 0.89 (t, J=7.4 Hz, 3H).

LCMS (ESI-TOF) m/z 510.2 [M+H⁺] with a purity of >97%.

### 2-Fluoroethyl 4-(4-chloro-2-(4-(2-hydroxypropan-2-yl)phenyl)quinoline-7-carbonyl)piperazine-1-carboxylate (B266)

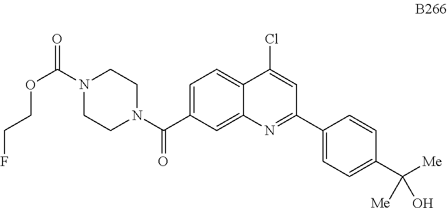 [SMILES: CC(C)(O)c1ccc(-c2cc(Cl)c3ccc(C(=O)N4CCN(C(=O)OCCF)CC4)cc3n2)cc1]

Compound B266 was synthesized according to General Procedure L using 2-fluoroethyl 4-(2,4-dichloroquinoline-7-carbonyl)piperazine-1-carboxylate and 4-(2-hydroxypropan-2-yl)phenylboronic acid as starting materials.

¹H NMR (400 MHz, DMSO-d₆) δ 8.46 (s, 1H), 8.30-8.22 (m, 3H), 8.14 (d, J=1.2 Hz, 1H), 7.74 (dd, J=8.5, 1.5 Hz, 1H), 7.66 (d, J=8.5 Hz, 2H), 5.14 (s, 1H), 4.61 (d, J=47.8 Hz, 2H), 4.37-4.19 (m, 2H), 3.79-3.35 (m, 8H), 1.48 (s, 6H).

LCMS (ESI-TOF) m/z 500.1 [M+H⁺] with a purity of >99%.

### 2-Fluoroethyl (R)-4-(4-chloro-2-(4-(2-hydroxypropan-2-yl)phenyl)quinoline-7-carbonyl)-2-methylpiperazine-1-carboxylate (B267)

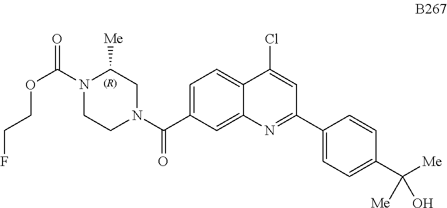

Compound B267 was synthesized according to General Procedure L using 2-fluoroethyl (R)-4-(2,4-dichloroquinoline-7-carbonyl)-2-methylpiperazine-1-carboxylate and 4-(2-hydroxypropan-2-yl)phenylboronic acid as starting materials.

¹H NMR (400 MHz, 80° C., DMSO-d₆) δ 8.36 (s, 1H), 8.27 (d, J=8.5 Hz, 1H), 8.22 (d, J=8.4 Hz, 2H), 8.10 (s, 1H), 7.71 (dd, J=8.5, 1.3 Hz, 1H), 7.65 (d, J=8.4 Hz, 2H), 4.88 (s, 1H), 4.59 (dt, J=47.8, 4.2 Hz, 2H), 4.34-3.63 (m, 8H), 3.36-3.12 (m, 3H), 1.50 (s, 6H), 1.15 (d, J=6.3 Hz, 3H).

LCMS (ESI-TOF) m/z 514.2 [M+H⁺] with a purity of >98%.

### Propyl (R)-4-(4-chloro-2-(1-methyl-1H-pyrrol-3-yl)quinoline-7-carbonyl)-2-methylpiperazine-1-carboxylate (B268)

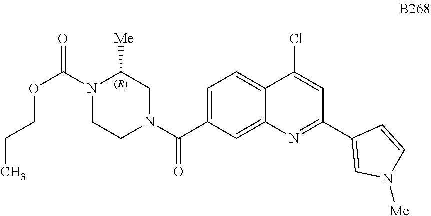

Compound B268 was synthesized according to General Procedure L using propyl (R)-4-(2,4-dichloroquinoline-7-carbonyl)-2-methylpiperazine-1-carboxylate and -1methylpyrrole-3-boronic acid pinacol ester as starting materials.

¹H NMR (400 MHz, 80° C., DMSO-d₆) δ 8.14 (d, J=8.4 Hz, 1H), 7.99 (s, 1H), 7.88 (d, J=1.1 Hz, 1H), 7.65 (s, 1H), 7.55 (dd, J=8.5, 1.5 Hz, 1H), 6.79 (dt, J=4.4, 2.6 Hz, 2H), 4.33-3.74 (m, 6H), 3.70 (s, 3H), 3.35-3.12 (m, 3H), 1.64-1.53 (m, 2H), 1.12 (d, J=6.1 Hz, 3H), 0.89 (t, J=7.4 Hz, 3H).

LCMS (ESI-TOF) m/z 455.2 [M+H⁺] with a purity of >99%.

### Propyl (R)-4-(4-chloro-2-(3-fluoro-4-(2-hydroxypropan-2-yl)phenyl)quinoline-7-carbonyl)-2-methylpiperazine-1-carboxylate (B269)

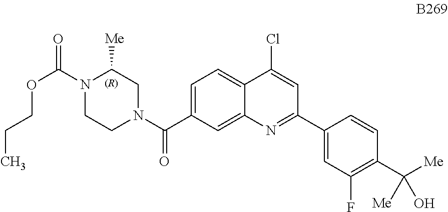

Compound B269 was synthesized according to General Procedure L using propyl (R)-4-(2,4-dichloroquinoline-7-carbonyl)-2-methylpiperazine-1-carboxylate and (3-fluoro-4-(2-hydroxypropan-2-yl)phenyl)boronic acid pinacol ester as starting materials.

¹H NMR (400 MHz, 80° C., DMSO-d₆) δ 8.42 (s, 1H), 8.28 (d, J=8.5 Hz, 1H), 8.11 (s, 1H), 8.11-8.07 (m, 1H), 8.02 (dd, J=13.5, 1.7 Hz, 1H), 7.82 (t, J=8.4 Hz, 1H), 7.73 (dd, J=8.5, 1.5 Hz, 1H), 5.14 (s, 1H), 4.31-3.75 (m, 6H), 3.38-3.12 (m, 3H), 1.61 (dd, J=14.1, 7.3 Hz, 2H), 1.56 (s, 6H), 1.14 (d, J=5.9 Hz, 3H), 0.89 (t, J=7.4 Hz, 3H).

LCMS (ESI-TOF) m/z 528.2 [M+H⁺] with a purity of >95%.

### Propyl (S)-4-(4-chloro-2-(4-(2-hydroxypropan-2-yl)phenyl)quinoline-7-carbonyl)-2-methylpiperazine-1-carboxylate (B270)

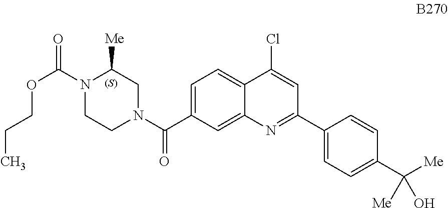

Compound B270 was synthesized according to General Procedure L using propyl (S)-4-(2,4-dichloroquinoline-7-carbonyl)-2-methylpiperazine-1-carboxylate and 4-(2-hydroxypropan-2-yl)phenylboronic acid as starting materials.

¹H NMR (400 MHz, 80° C., DMSO-d₆) δ 8.36 (s, 1H), 8.27 (d, J=8.6 Hz, 1H), 8.22 (d, J=8.5 Hz, 2H), 8.09 (d, J=1.1 Hz, 1H), 7.71 (dd, J=8.5, 1.5 Hz, 1H), 7.65 (d, J=8.5 Hz, 2H), 4.87 (s, 1H), 4.31-3.76 (m, 6H), 3.39-3.10 (m, 3H), 1.66-1.54 (m, 2H), 1.50 (s, 6H), 1.14 (d, J=6.7 Hz, 3H), 0.89 (t, J=7.4 Hz, 3H).

LCMS (ESI-TOF) m/z 510.2 [M+H⁺] with a purity of >99%.

### Propyl 4-(4-chloro-2-(1-(oxetan-3-yl)-1H-pyrazol-4-yl)quinoline-7-carbonyl)piperazine-1-carboxylate (B271)

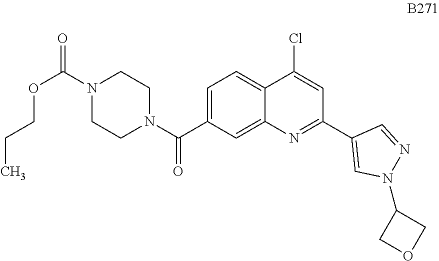 [SMILES: CCCOC(=O)N1CCN(C(=O)c2ccc3c(Cl)cc(-c4cnn(C5COC5)c4)nc3c2)CC1]

Compound B271 was synthesized according to General Procedure L using propyl 4-(2,4-dichloroquinoline-7-carbonyl)piperazine-1-carboxylate and 1-(3-oxetanyl)-1H-Pyrazole-4-boronic acid pinacol ester as starting materials.

¹H NMR (400 MHz, DMSO-d₆) δ 8.78 (s, 1H), 8.39 (s, 1H), 8.27 (s, 1H), 8.21 (d, J=8.5 Hz, 1H), 7.99 (d, J=0.8 Hz, 1H), 7.67 (dd, J=8.6, 1.4 Hz, 1H), 5.67 (p, J=7.4 Hz, 1H), 4.96 (p, J=6.8 Hz, 4H), 3.98 (t, J=6.6 Hz, 2H), 3.77-3.35 (m, 8H), 1.64-1.52 (m, 2H), 0.89 (t, J=7.5 Hz, 3H).

LCMS (ESI-TOF) m/z 484.1 [M+H⁺] with a purity of >99%.

### Propyl (R)-4-(4-chloro-2-(1-(oxetan-3-yl)-1H-pyrazol-4-yl)quinoline-7-carbonyl)-2-methylpiperazine-1-carboxylate (B272)

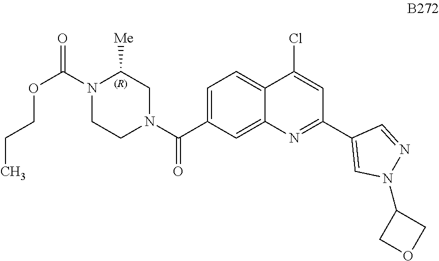

Compound B272 was synthesized according to General Procedure L using propyl (R)-4-(2,4-dichloroquinoline-7-carbonyl)-2-methylpiperazine-1-carboxylate and 1-(3-oxetanyl)-1H-pyrazole-4-boronic acid pinacol ester as starting materials.

¹H NMR (400 MHz, 80° C., DMSO-d₆) δ 8.71 (s, 1H), 8.33 (s, 1H), 8.21 (d, J=8.4 Hz, 1H), 8.18 (s, 1H), 7.96 (d, J=1.0 Hz, 1H), 7.64 (dd, J=8.5, 1.4 Hz, 1H), 5.64 (p, J=6.7 Hz, 1H), 4.97 (p, J=6.7 Hz, 4H), 4.29-3.73 (m, 6H), 3.34-3.09 (m, 3H), 1.67-1.52 (m, 2H), 1.13 (d, J=6.0 Hz, 3H), 0.89 (t, J=7.4 Hz, 3H).

LCMS (ESI-TOF) m/z 498.2 [M+H⁺] with a purity of >98%.

### Propyl (S)-4-(4-chloro-2-(1-(oxetan-3-yl)-1H-pyrazol-4-yl)quinoline-7-carbonyl)-2-methylpiperazine-1-carboxylate (B273)

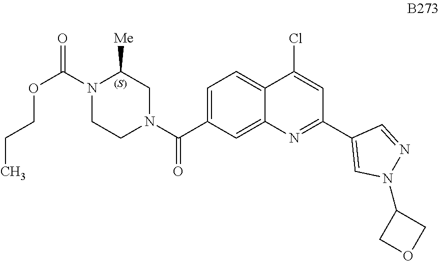

Compound B273 was synthesized according to General Procedure L using propyl (S)-4-(2,4-dichloroquinoline-7-carbonyl)-2-methylpiperazine-1-carboxylate and 1-(3-oxetanyl)-1H-pyrazol-4-boronic acid pinacol ester as starting materials.

¹H NMR (400 MHz, 80° C., DMSO-d₆) δ 8.71 (s, 1H), 8.33 (s, 1H), 8.20 (t, J=7.6 Hz, 1H), 8.17 (s, 1H), 7.96 (s, 1H), 7.64 (dd, J=8.5, 1.3 Hz, 1H), 5.71-5.54 (m, 1H), 4.97 (p, J=6.7 Hz, 4H), 4.33-3.59 (m, 6H), 3.36-3.07 (m, 3H), 1.66-1.52 (m, 2H), 1.13 (d, J=6.4 Hz, 3H), 0.89 (t, J=7.4 Hz, 3H).

LCMS (ESI-TOF) m/z 498.2 [M+H⁺] with a purity of >97%.

### Propyl 4-(4-chloro-2-(1-(2,2,2-trifluoroethyl)-1H-pyrazol-4-yl)quinoline-7-carbonyl)piperazine-1-carboxylate (B274)

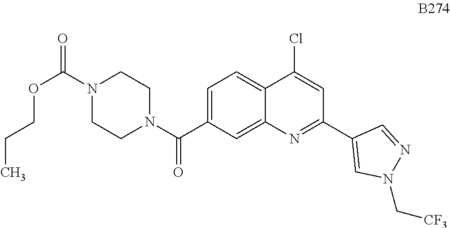 [SMILES: CCCOC(=O)N1CCN(C(=O)c2ccc3c(Cl)cc(-c4cnn(CC(F)(F)F)c4)nc3c2)CC1]

Compound B274 was synthesized according to General Procedure L using propyl 4-(2,4-dichloroquinoline-7-carbonyl)piperazine-1-carboxylate and (1-(2,2,2-trifluoroethyl)-1H-pyrazol-4-yl)boronic acid as starting materials.

¹H NMR (400 MHz, DMSO-d₆) δ 8.70 (s, 1H), 8.41 (s, 1H), 8.30 (s, 1H), 8.23 (d, J=8.4 Hz, 1H), 8.01 (s, 1H), 7.68 (dd, J=8.5, 1.4 Hz, 1H), 5.27 (q, J=9.3 Hz, 2H), 3.98 (t, J=6.6 Hz, 2H), 3.75-3.37 (m, 8H), 1.59 (dd, J=13.2, 6.1 Hz, 2H), 0.89 (t, J=7.4 Hz, 3H).

LCMS (ESI-TOF) m/z 510.1 [M+H⁺] with a purity of >98%.

### Propyl (R)-4-(4-chloro-2-(1-(2,2,2-trifluoroethyl)-1H-pyrazol-4-yl)quinoline-7-carbonyl)-2-methylpiperazine-1-carboxylate (B275)

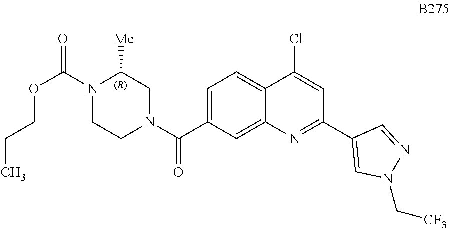

Compound B275 was synthesized according to General Procedure L using propyl (R)-4-(2,4-dichloroquinoline-7-carbonyl)-2-methylpiperazine-1-carboxylate and (1-(2,2,2-trifluoroethyl)-1H-pyrazol-4-yl)boronic acid as starting materials.

¹H NMR (400 MHz, 80° C., DMSO-d₆) δ 8.66 (s, 1H), 8.35 (s, 1H), 8.22 (d, J=8.4 Hz, 1H), 8.20 (s, 1H), 7.98 (d, J=1.2 Hz, 1H), 7.66 (dd, J=8.3, 1.1 Hz, 1H), 5.19 (q, J=9.1 Hz, 2H), 4.33-3.69 (m, 6H), 3.32-3.09 (m, 3H), 1.67-1.52 (m, 2H), 1.13 (d, J=6.6 Hz, 3H), 0.89 (t, J=7.4 Hz, 3H).

LCMS (ESI-TOF) m/z 524.1 [M+H⁺] with a purity of >96%.

### Propyl (S)-4-(4-chloro-2-(1-(2,2,2-trifluoroethyl)-1H-pyrazol-4-yl)quinoline-7-carbonyl)-2-methylpiperazine-1-carboxylate (B276)

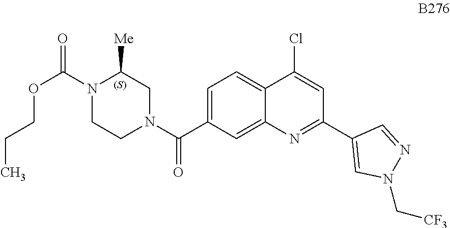

Compound B276 was synthesized according to General Procedure L using propyl (S)-4-(2,4-dichloroquinoline-7-carbonyl)-2-methylpiperazine-1-carboxylate and (1-(2,2,2-trifluoroethyl)-1H-pyrazol-4-yl) boronic acid as starting materials.

¹H NMR (400 MHz, 80° C., DMSO-d₆) δ 8.65 (s, 1H), 8.35 (s, 1H), 8.22 (d, J=8.5 Hz, 1H), 8.19 (s, 1H), 7.98 (d, J=0.8 Hz, 1H), 7.65 (dd, J=8.2, 1.3 Hz, 1H), 5.28-5.08 (m, 2H), 4.30-3.73 (m, 6 H), 3.36-3.09 (m, 3H), 1.66-1.47 (m, 2H), 1.13 (d, J=6.2 Hz, 3H), 0.89 (t, J=7.4 Hz, 3H).

LCMS (ESI-TOF) m/z 524.1 [M+H⁺] with a purity of >95%.

### Propyl 4-(4-chloro-2-(1-cyclopentyl-1H-pyrazol-4-yl)quinoline-7-carbonyl)piperazine-1-carboxylate (B277)

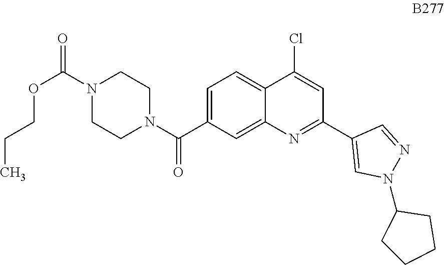 [SMILES: CCCOC(=O)N1CCN(C(=O)c2ccc3c(Cl)cc(-c4cnn(C5CCCC5)c4)nc3c2)CC1]

Compound B277 was synthesized according to General Procedure L using propyl 4-(2,4-dichloroquinoline-7-carbonyl)piperazine-1-carboxylate and 1-cyclopentyl-1H-pyrazole-4-boronic acid as starting materials.

¹H NMR (400 MHz, DMSO-d₆) δ 8.63 (s, 1H), 8.24 (d, J=1.3 Hz, 2H), 8.20 (d, J=8.4 Hz, 1H), 7.98 (s, 1H), 7.65 (d, J=8.6 Hz, 1H), 4.84-4.68 (m, 1H), 3.98 (t, J=6.7 Hz, 2H), 3.77-3.37 (m, 8H), 2.21-2.08 (m, 2H), 2.05-1.93 (m, 2H), 1.89-1.77 (m, 2H), 1.72-1.63 (m, 2H), 1.63-1.49 (m, 2H), 0.89 (t, J=7.2 Hz, 3H).

LCMS (ESI-TOF) m/z 496.2 [M+H⁺] with a purity of >96%.

### Propyl (R)-4-(4-chloro-2-(1-cyclopentyl-1H-pyrazol-4-yl)quinoline-7-carbonyl)-2-methylpiperazine-1-carboxylate (B278)

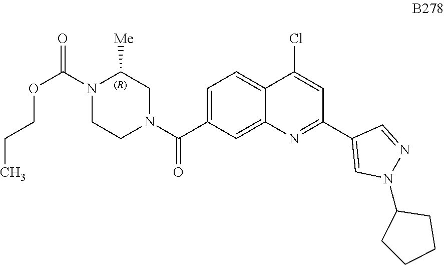

Compound B278 was synthesized according to General Procedure L using propyl (R)-4-(2,4-dichloroquinoline-7-carbonyl)-2-methylpiperazine-1-carboxylate and 1-cyclopentyl-1H-pyrazole-4-boronic acid as starting materials.

¹H NMR (400 MHz, 80° C., DMSO-d₆) δ 8.55 (s, 1H), 8.23-8.17 (m, 2H), 8.15 (s, 1H), 7.95 (d, J=1.0 Hz, 1H), 7.62 (dd, J=8.8, 1.2 Hz, 1H), 4.82-4.70 (m, 1H), 4.33-3.74 (m, 6H), 3.36-3.15 (m, 3H), 2.23-2.09 (m, 2H), 2.08-1.93 (m, 2H), 1.89-1.75 (m, 2H), 1.75-1.65 (m, 2H), 1.59 (dd, J=14.2, 7.4 Hz, 2H), 1.12 (d, J=4.5 Hz, 3H), 0.89 (t, J=7.4 Hz, 3H).

LCMS (ESI-TOF) m/z 510.2 [M+H⁺] with a purity of >99%.

### Propyl (S)-4-(4-chloro-2-(1-cyclopentyl-1H-pyrazol-4-yl)quinoline-7-carbonyl)-2-methylpiperazine-1-carboxylate (B279)

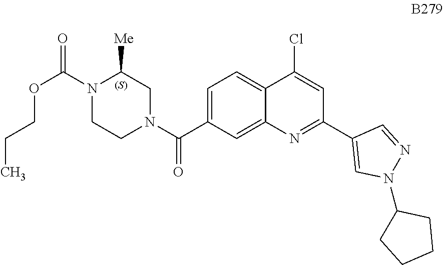

Compound B279 was synthesized according to General Procedure L using propyl (S)-4-(2,4-dichloroquinoline-7-carbonyl)-2-methylpiperazine-1-carboxylate and 1-cyclopentyl-1H-pyrazole-4-boronic acid as starting materials.

¹H NMR (400 MHz, 80° C., DMSO-d₆) δ 8.55 (s, 1H), 8.23-8.17 (m, 2H), 8.15 (s, 1H), 7.95 (d, J=1.0 Hz, 1H), 7.62 (dd, J=8.8, 1.2 Hz, 1H), 4.82-4.70 (m, 1H), 4.33-3.74 (m, 6H), 3.36-3.15 (m, 3H), 2.23-2.09 (m, 2H), 2.08-1.93 (m, 2H), 1.89-1.75 (m, 2H), 1.75-1.65 (m, 2H), 1.59 (dd, J=14.2, 7.4 Hz, 2H), 1.12 (d, J=4.5 Hz, 3H), 0.89 (t, J=7.4 Hz, 3H).

LCMS (ESI-TOF) m/z 510.2 [M+H⁺] with a purity of >99%.

### Propyl (S)-4-(2-(3-(aminomethyl)-4-methoxyphenyl)-4-chloroquinoline-7-carbonyl)-2-methylpiperazine-1-carboxylate (B280)

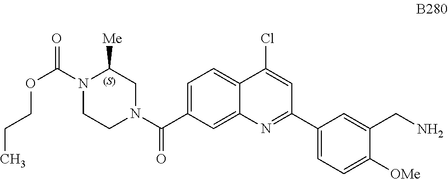

Compound B280 was synthesized according to General Procedure L and then General Procedure D using propyl (S)-4-(2,4-dichloroquinoline-7-carbonyl)-2-methylpiperazine-1-carboxylate and 3-cyano-4-methoxyphenyl boronic acid as starting materials (General Procedure L).

¹H NMR (400 MHz, 80° C., DMSO-d₆) δ 8.34 (s, 1H), 8.28 (d, J=2.3 Hz, 1H), 8.24 (d, J=8.5 Hz, 1H), 8.18 (dd, J=8.6, 2.3 Hz, 1H), 8.07 (d, J=1.2 Hz, 1H), 7.67 (dd, J=8.5, 1.5 Hz, 1H), 7.12 (d, J=8.5 Hz, 1H), 4.33-3.94 (m, 4H), 3.90 (s, 3H), 3.86-3.74 (m, 4H), 3.35-3.10 (m, 3H), 1.68 (s, 2H), 1.59 (dq, J=14.0, 7.2 Hz, 2H), 1.14 (d, J=6.0 Hz, 3H), 0.89 (t, J=7.4 Hz, 3H).

LCMS (ESI-TOF) m/z 511.2 [M+H⁺] with a purity of >99%.

### Propyl (R)-4-(2-(4-(1-aminocyclopropyl)phenyl)-4-chloroquinoline-7-carbonyl)-2-methylpiperazine-1-carboxylate (B281)

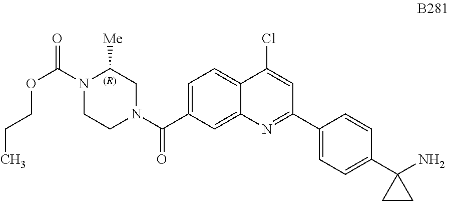

Compound B281 was synthesized according to General Procedure L using propyl (R)-4-(2,4-dichloroquinoline-7-carbonyl)-2-methylpiperazine-1-carboxylate and 4-(1-aminocyclopropyl)phenylboronic acid hydrochloride as starting materials.

¹H NMR (400 MHz, 80° C., DMSO-d₆) δ 8.35 (s, 1H), 8.26 (d, J=8.5 Hz, 1H), 8.20 (d, J=8.5 Hz, 2H), 8.08 (d, J=1.0 Hz, 1H), 7.70 (dd, J=8.5, 1.4 Hz, 1H), 7.49 (d, J=8.5 Hz, 2H), 4.33-3.72 (m, 6H), 3.37-3.10 (m, 3H), 2.31 (s, 2H), 1.66-1.52 (m, 2H), 1.14 (d, J=6.4 Hz, 3H), 1.07-0.96 (m, 4H), 0.89 (t, J=7.4 Hz, 3H).

LCMS (ESI-TOF) m/z 507.2 [M+H⁺] with a purity of >98%.

### Propyl (R)-4-(2-(4-(1-amino-2-methylpropan-2-yl)phenyl)-4-chloroquinoline-7-carbonyl)-2-methylpiperazine-1-carboxylate (B282)

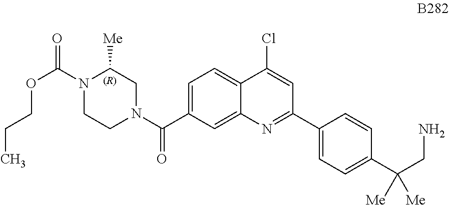

Compound B282 was synthesized according to General Procedure L using propyl (R)-4-(2,4-dichloroquinoline-7-carbonyl)-2-methylpiperazine-1-carboxylate and (4-(1-amino-2-methylpropan-2-yl)phenyl)boronic acid hydrochloride as starting materials.

¹H NMR (400 MHz, 80° C., DMSO-d₆) δ 8.34 (s, 1H), 8.27 (d, J=8.5 Hz, 1H), 8.21 (d, J=8.5 Hz, 2H), 8.09 (d, J=1.1 Hz, 1H), 7.70 (dd, J=8.5, 1.6 Hz, 1H), 7.54 (d, J=8.5 Hz, 2H), 4.33-3.72 (m, 6H), 3.37-3.10 (m, 3H), 2.75 (s, 2H), 1.69-1.52 (m, 2H), 1.30 (s, 6H), 1.14 (d, J=6.5 Hz, 5H), 0.89 (t, J=7.4 Hz, 3H).

LCMS (ESI-TOF) m/z 523.3 [M+H⁺] with a purity of >96%.

### Propyl (2R)-4-(4-chloro-2-(4-(1-(pyrrolidin-1-yl)ethyl)phenyl)quinoline-7-carbonyl)-2-methylpiperazine-1-carboxylate (B283)

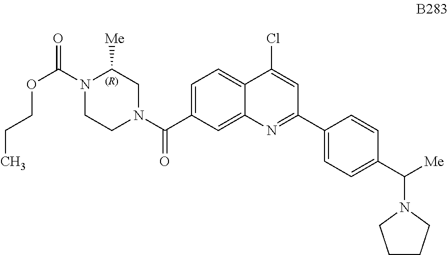

Compound B283 was synthesized according to General Procedure L using propyl (R)-4-(2,4-dichloroquinoline-7-carbonyl)-2-methylpiperazine-1-carboxylate and 4-(1-pyrrolidinoethyl)phenylboronic acid as starting materials.

¹H NMR (400 MHz, 80° C., DMSO-d₆) δ 8.35 (s, 1H), 8.27 (d, J=8.5 Hz, 1H), 8.22 (d, J=8.3 Hz, 2H), 8.09 (d, J=0.6 Hz, 1H), 7.71 (dd, J=8.6, 1.4 Hz, 1H), 7.49 (d, J=8.3 Hz, 2H), 4.33-3.72 (m, 6H), 3.36 (q, J=6.3 Hz, 1H), 3.32-3.10 (m, 3H), 2.60-2.52 (m, 2H), 2.42-2.33 (m, 2H), 1.69 (s, 4H), 1.64-1.54 (m, 2H), 1.36 (d, J=6.6 Hz, 3H), 1.14 (d, J=6.4 Hz, 3H), 0.89 (t, J=7.4 Hz, 3H).

LCMS (ESI-TOF) m/z 549.3 [M+H⁺] with a purity of >97%.

### Propyl (2R)-4-(4-chloro-2-(4-(1-(dimethylamino)ethyl)phenyl)quinoline-7-carbonyl)-2-methylpiperazine-1-carboxylate (B284)

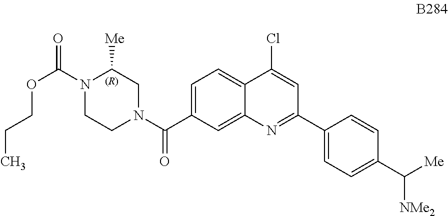

Compound B284 was synthesized according to General Procedure L using propyl (R)-4-(2,4-dichloroquinoline-7-carbonyl)-2-methylpiperazine-1-carboxylate and {4-[1-(dimethylamino)ethyl]phenyl}boronic acid hydrochloride as starting materials.

¹H NMR (400 MHz, 80° C., DMSO-d₆) δ 8.36 (s, 1H), 8.27 (d, J=8.5 Hz, 1H), 8.23 (d, J=8.2 Hz, 2H), 8.09 (d, J=0.9 Hz, 1H), 7.71 (dd, J=8.6, 1.2 Hz, 1H), 7.48 (d, J=8.3 Hz, 2H), 4.33-3.72 (m, 6H), 3.41 (q, J=6.6 Hz, 1H), 3.36-3.10 (m, 3H), 2.16 (s, 6H), 1.67-1.53 (m, 2H), 1.33 (d, J=6.7 Hz, 3H), 1.14 (d, J=6.8 Hz, 3H), 0.89 (t, J=7.4 Hz, 3H).

LCMS (ESI-TOF) m/z 523.2 [M+H⁺] with a purity of >97%.

### Propyl 4-(2-(4-(1-amino-2-methylpropan-2-yl)phenyl)-4-chloroquinoline-7-carbonyl)piperazine-1-carboxylate (B285)

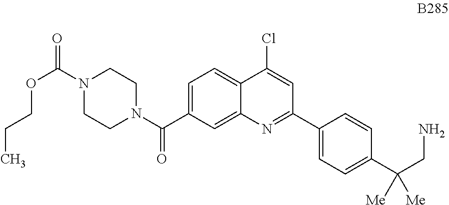 [SMILES: CCCOC(=O)N1CCN(C(=O)c2ccc3c(Cl)cc(-c4ccc(C(C)(C)CN)cc4)nc3c2)CC1]

Compound B285 was synthesized according to General Procedure L using propyl 4-(2,4-dichloroquinoline-7-carbonyl)piperazine-1-carboxylate and (4-(1-amino-2-methylpropan -2-yl)phenyl)boronic acid hydrochloride as starting materials.

LCMS (ESI-TOF) m/z 509.2 [M+H⁺] with a purity of >99%.

### Propyl 4-(4-chloro-2-(4-(2-methyl-1-(methylamino)propan-2-yl)phenyl)quinoline-7-carbonyl)piperazine-1-carboxylate (B286)

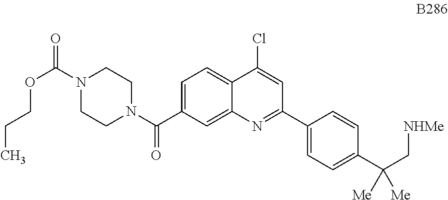 [SMILES: CCCOC(=O)N1CCN(C(=O)c2ccc3c(Cl)cc(-c4ccc(C(C)(C)CNC)cc4)nc3c2)CC1]

Step 1: Compound B285 (90 mg, 0.177 mmol) was dissolved in dichloromethane (2 mL) and triethylamine (50 μL, 0.354 mmol, 2 equiv). The mixture was cooled to 0° C. before adding di-tert-butyl dicarbonate (46.3 mg, 0.212 mmol, 1.2 equiv) was added. After stirring for 30 min at room temperature, the mixture was quenched by addition of saturated ammonium chloride. The organic layer was removed and the aqueous layer was extracted twice with dichloromethane. The combined organic layer was dried over anhydrous sodium sulfate, filtered and concentrated under reduced pressure. The crude material was purified by column chromatography (0-50% ethyl acetate/hexanes) to afford propyl 4-(2-(4-(1-((tert-butoxycarbonyl)amino)-2-methylpropan-2-yl)phenyl)-4-chloroquinoline-7-carbonyl)piperazine-1-carboxylate as a white solid (101.7 mg, 94%).

Step 2: The intermediate above (100 mg, 0.164 mmol) was dissolved in N,N-dimethylformamide (1.5 mL) and cooled to 0° C. Upon addition of sodium hydride, 60% dispersion in mineral oil (7.8 mg, 0.197 mmol, 1.2 equiv), the mixture was allowed to stir at room temperature for 30 min. Iodomethane (20 μL, 0.32 mmol, 2 equiv) was added dropwise and the mixture was allowed to stir for 2 h before quenching with saturated sodium bicarbonate. The aqueous layer was extracted with ethyl acetate and washed with brine, dried over anhydrous sodium sulfate, filtered and concentrated under reduced pressure. The crude material was used without further purification.

Step 3: The residue was dissolved in dichloromethane (0.1 mL) and trifluoroacetic acid (0.1 mL). After 10 min, the crude material was purified using preparative HPLC (35% acetonitrile/water; 0.1% formic acid) to afford compound B286) as a white solid (32 mg, 37%).

NMR (400 MHz, DMSO-d₆) δ 8.44 (s, 1H), 8.27 (d, J=8.6 Hz, 1H), 8.24 (d, J=8.5 Hz, 2H), 8.12 (d, J=1.2 Hz, 1H), 7.74 (dd, J=8.5, 1.5 Hz, 1H), 7.55 (d, J=8.5 Hz, 2H), 3.98 (t, J=6.6 Hz, 2H), 3.75-3.37 (m, 8H), 2.66 (s, 2H), 2.24 (s, 3H), 1.59 (dd, J=11.9, 4.9 Hz, 2H), 1.32 (s, 6H), 0.89 (t, J=7.4 Hz, 3H).

LCMS (ESI-TOF) m/z 523.2 [M+H⁺] with a purity of >99%.

### Propyl 4-(4-chloro-2-(4-(1-(dimethylamino)-2-methylpropan-2-yl)phenyl)quinoline-7-carbonyl)piperazine-1-carboxylate (B287)

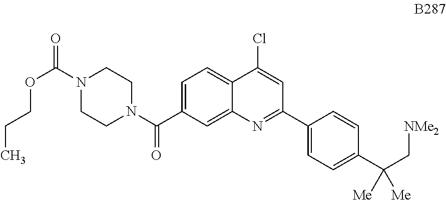 [SMILES: CCCOC(=O)N1CCN(C(=O)c2ccc3c(Cl)cc(-c4ccc(C(C)(C)CN(C)C)cc4)nc3c2)CC1]

Compound B285 (90 mg, 0.177 mmol) was dissolved in methanol (0.9 mL) and paraformaldehyde (106 mg) was added. After 90 min, sodium borohydride (33.5 mg, 0.885 mmol, 5 equiv) was added and the mixture was quenched with 2M hydrochloric acid after 30 min to pH 1. The mixture was purified by preparative HPLC (35% acetonitrile/water; 0.1% formic acid) to give a mixture of compound 285, 286 and 287 as a white solid (19.1 mg). The mixture was re-dissolved in dichloromethane (0.3 mL) and triethylamine (5 μL, 0.036 mmol). Di-tert-butyl dicarbonate (6 mg, 0.027 mmol) was then added. After 30 min, the mixture was quenched with saturated ammonium chloride, and was extracted twice with dichloromethane. The combined organic layer was dried over anhydrous sodium sulfate, filtered and concentrated under reduced pressure. The crude material was purified by column chromatography (0-4% methanol/dichloromethane) to afford compound B287 as white solid upon lyophilisation (12.1 mg, 13%).

¹H NMR (400 MHz, DMSO-d₆) δ 8.45 (s, 1H), 8.29-8.20 (m, 3H), 8.12 (s, 1H), 7.73 (dd, J=8.5, 1.5 Hz, 1H), 7.57 (d, J=8.4 Hz, 2H), 3.98 (t, J=6.6 Hz, 2H), 3.78-3.34 (m, 8H), 2.02 (s, 6H), 1.59 (dd, J=13.5, 6.4 Hz, 2H), 1.33 (s, 6H), 0.89 (t, J=6.9 Hz, 3H).

LCMS (ESI-TOF) m/z 537.2 [M+H⁺] with a purity of >99%.

### (R)-5-(2((R)-4-(4-Chloro-2-(1-methyl-1H-pyrrol-3-yl)quinoline-7-carbonyl)-2-methylpiperazin-1-yl)-2-oxoethyl)pyrrolidin-2-one (D001)

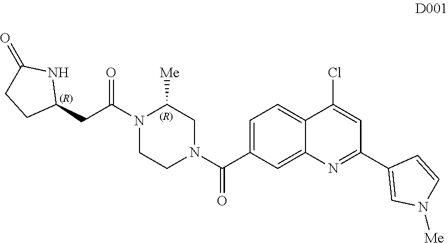

Step 1: Tert-butyl (R)-4-(2,4-dichloroquinoline-7-carbonyl)-2-methylpiperazine-1-carboxylate was synthesized according to General Procedure C1 using compound S9 (where R¹=H) and tert-butyl (R)-2-methylpiperazine-1-carboxylate as starting materials.

Step 2: (R)-(2,4-Dichloroquinolin-7-yl)(3-methylpiperazin-1-yl)methanone was synthesized by subjecting the intermediate above to 1:1 trifluoroacetic acid/dichloromethane mixture for 10 min followed by a basic work-up. The crude material was used directly without further purification.

Step 3: (R)-5-(2-((R)-4-(2,4-Dichloroquinoline-7-carbonyl)-2-methylpiperazin-1-yl)-2-oxoethyl)pyrrolidin-2-one was synthesized according to General Procedure C1 using (R)-2-(5-oxopyrrolidin-2-yl)acetic acid as starting material.

Step 4: Compound D001 was synthesized according to General Procedure L by using 1-methylpyrrole-3-boronic acid pinacol ester as starting material.

¹H NMR (400 MHz, 80° C., DMSO-d₆) δ 8.15 (d, J=8.5 Hz, 1H), 7.99 (s, 1H), 7.89 (d, J=0.7 Hz, 1H), 7.65 (s, 1H), 7.56 (dd, J=8.4, 1.2 Hz, 1H), 7.08 (s, 1H), 6.84-6.72 (m, 2H), 5.68 (s, 1H), 4.62-3.51 (m, 8H), 3.26 (s, 1H), 2.25-1.98 (m, 3H), 1.72-1.57 (m, 1H), 1.25 (s, 2H), 1.13 (s, 3H), 0.92-0.83 (m, 1H).

LCMS (ESI-TOF) m/z 494.1 [M+H⁺] with a purity of >95%.

### (4-Chloro-2-phenylquinolin-7-yl)(4-(1-cyclopropyl-1H-1,2,3-triazole-4-carbonyl)piperazin-1-yl)methanone (D002)

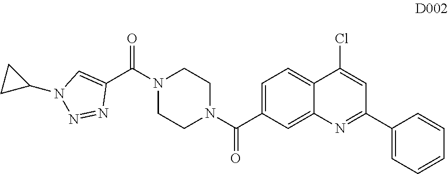 [SMILES: O=C(c1ccc2c(Cl)cc(-c3ccccc3)nc2c1)N1CCN(C(=O)c2cn(C3CC3)nn2)CC1]

Step 1: 4-Chloro-2-phenylquinoline-7-carboxylic acid was synthesized according to General Procedure I and K using acetophenone as starting material.

Step 2: (1-Cyclopropyl-1H-1,2,3-triazol-4-yl)(piperazin-1-yl)methanone was synthesized according to General Procedure C1 using 1-cyclopropyl-1H-1,2,3-triazole-4-carboxylic acid and tert-butyl piperazine-1-carboxylate as starting materials, followed by treatment with 1:1 trifluoroacetic acid/dichloromethane for 10 min.

Step 3: Compound D002 was synthesized according to General Procedure C1 using intermediates from Step 1 and Step 2 as coupling partners.

¹H NMR (400 MHz, DMSO-d₆) δ 8.61 (s, 1H), 8.49 (s, 1H), 8.34 (dd, J=7.9, 1.6 Hz, 2H), 8.30 (d, J=8.5 Hz, 1H), 8.18 (d, J=1.2 Hz, 1H), 7.79 (dd, J=8.5, 1.6 Hz, 1H), 7.63-7.53 (m, 3H), 4.29-3.40 (m, 9H), 1.32-1.07 (m, 4H).

LCMS (ESI-TOF) m/z 487.2 [M+H⁺] with a purity of >99%.

### (4-Chloro-2-(1-methyl-1H-pyrrol-3-yl)quinolin-7-yl)(4-(1-cyclopropyl-1H-1,2,3-triazole-4-carbonyl)piperazin-1-yl)methanone (D003)

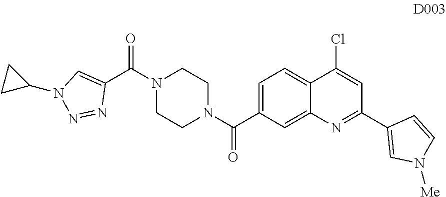 [SMILES: Cn1ccc(-c2cc(Cl)c3ccc(C(=O)N4CCN(C(=O)c5cn(C6CC6)nn5)CC4)cc3n2)c1]

Step 1: 4-Chloro-2-(1-methyl-1H-pyrrol-3-yl)quinoline-7-carboxylic acid was synthesized according to General Procedure I and K using 3-acetyl-1-methylpyrrole as starting material.

Step 2: Compound D003 was synthesized according to General Procedure C1 using intermediate 1 and (1-cyclopropyl-1H-1,2,3-triazol-4-yl)(piperazin-1-yl)methanone as starting materials.

¹H NMR (400 MHz, DMSO-d₆) δ 8.60 (s, 1H), 8.15 (d, J=8.6 Hz, 1H), 8.07 (s, 1H), 7.94 (d, J=1.1 Hz, 1H), 7.71 (t, J=1.7 Hz, 1H), 7.61 (dd, J=8.5, 1.5 Hz, 1H), 6.88-6.77 (m, 2H), 4.30-3.40 (m, 12H), 1.31-1.06 (m, 4H).

LCMS (ESI-TOF) m/z 490.2 [M+H⁺] with a purity of >99%.

### (R)-(4-Chloro-2-(1-methyl-1H-pyrrol-3-yl)quinolin-7-yl)(4-(1-cyclopropyl-1H-1,2,3-triazole-4-carbonyl)-3-methylpiperazin-1-yl)methanone (D004)

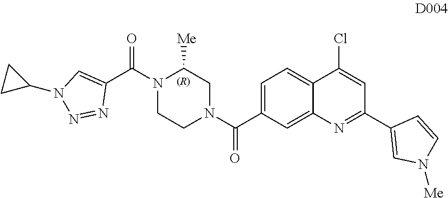

Step 1: (R)-(1-Cyclopropyl-1H-1,2,3-triazol-4-yl)(2-methylpiperazin-1-yl)methanone was synthesized according to General Procedure C1 using 1-cyclopropyl-1H-1,2,3-triazole-4-carboxylic acid and tert-butyl (R)-3-methylpiperazine-1-carboxylate as starting materials, followed by treatment with 1:1 trifluoroacetic acid/dichloromethane for 10 min.

Step 2: Compound D004 was synthesized according to General Procedure C1 using 4-chloro-2-(1-methyl-1H-pyrrol-3-yl)quinoline-7-carboxylic acid and intermediate from Step 1 as starting materials.

¹H NMR (400 MHz, 80° C., DMSO-d₆) δ 8.47 (s, 1H), 8.15 (d, J=8.5 Hz, 1H), 7.99 (s, 1H), 7.91 (d, J=1.2 Hz, 1H), 7.65 (t, J=1.8 Hz, 1H), 7.57 (dd, J=8.5, 1.5 Hz, 1H), 6.80 (dt, J=4.5, 2.7 Hz, 2H), 4.90 (br s, 1H), 4.50 (br s, 1H), 4.21-3.81 (m, 3H), 3.71 (s, 3H), 3.35 (br s, 2H), 3.26-3.11 (m, 1H), 1.30-1.05 (m, 7H).

LCMS (ESI-TOF) m/z 504.2 [M+H⁺] with a purity of >99%.

### (R)-(4-Chloro-2-phenylquinolin-7-yl)(4-(1-cyclopropyl-1H-1,2,3-triazole-4-carbonyl)-3-methylpiperazin-1-yl)methanone (D005)

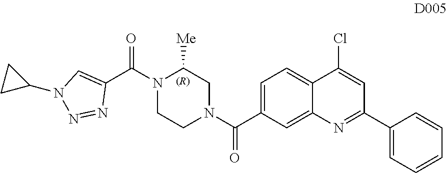

Compound D005 was synthesized according to General Procedure C1 using 4-chloro-2-phenylquinoline-7-carboxylic acid and (R)-(1-cyclopropyl-1H-1,2,3-triazol-4-yl)(2-methylpiperazin-1-yl)methanone as starting materials.

¹H NMR (400 MHz, 80° C., DMSO-d₆) δ 8.46 (s, 1H), 8.38 (s, 1H), 8.32-8.26 (m, 3H), 8.15-8.12 (m, 1H), 7.75 (dd, J=8.6, 1.3 Hz, 1H), 7.61-7.49 (m, 3H), 4.91 (br s, 1H), 4.51 (br s, 1H), 4.21-3.70 (m, 3H), 3.38 (br s, 2H), 3.20 (s, 1H), 1.34-1.07 (m, 7H).

LCMS (ESI-TOF) m/z 501.2 [M+H⁺] with a purity of >99%.

### (R)-(4-Chloro-2-(4-(2-hydroxypropan-2-yl)phenyl)quinolin-7-yl)(4-(1-cyclopropyl-1H-1,2,3-triazole-4-carbonyl)-3-methylpiperazin-1-yl)methanone (D006)

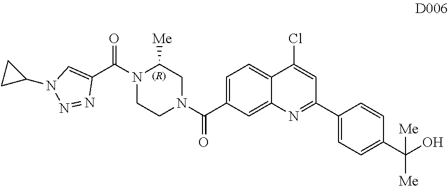

Step 1: (R)-(1-Cyclopropyl-1H-1,2,3-triazol-4-yl)(4-(2,4-dichloroquinoline-7-carbonyl)-2-methylpiperazin-1-yl)methanone was synthesized according to General Procedure C1 using compound S9 (where R¹=H) and (R)-(1-cyclopropyl-1H-1,2,3-triazol-4-yl)(2-methylpiperazin-1-yl)methanone as starting materials.

Step 2: Compound D006 was synthesized according to General Procedure L using the above intermediate and 4-(2-hydroxypropan-2-yl.)phenylboronic acid as starting materials.

¹H NMR (400 MHz, 80° C., DMSO-d₆) δ 8.47 (s, 1H), 8.36 (s, 1H), 8.28 (d, J=8.5 Hz, 1H), 8.22 (d, J=8.5 Hz, 2H), 8.12 (d, J=1.0 Hz, 1H), 7.73 (dd, J=8.6, 1.4 Hz, 1H), 7.65 (d, J=8.5 Hz, 2H), 5.00-4.80 (m, 2H), 4.50 (br s, 1H), 4.30-3.68 (m, 3H), 3.37 (br s, 2H), 3.20 (t, J=13.3 Hz, 1H), 1.50 (s, 6H), 1.32-1.06 (m, 7H).

LCMS (ESI-TOF) m/z 559.2 [M+H⁺] with a purity of >98%.

### (4-Chloro-2-phenylquinolin-7-yl)(4-(1-methyl-1H-1,2,3-triazole-4-carbonyl)piperazin-1-yl)methanone (D007)

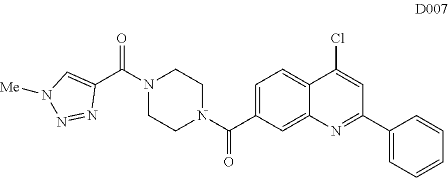 [SMILES: Cn1cc(C(=O)N2CCN(C(=O)c3ccc4c(Cl)cc(-c5ccccc5)nc4c3)CC2)nn1]

Step 1: (1-Methyl-1H-1,2,3-triazol-4-yl)(piperazin-1-yl)methanone was synthesized according to General Procedure C1 using 1-methyl-1H-1,2,3-trizole-4-carboxylic acid and tert-butyl piperazine-1-carboxylate as starting materials, followed by treatment with 1:1 trifluoroacetic acid/dichloromethane.

Step 2: Compound D007 was synthesized according to General Procedure C1 using intermediate from Step 1 and 4-chloro-2-phenylquinoline-7-carboxylic acid as starting materials.

¹H NMR (400 MHz, DMSO-d₆) δ 8.53 (s, 1H), 8.50 (s, 1H), 8.34 (dd, J=7.7, 1.5 Hz, 2H), 8.30 (d, J=8.4 Hz, 1H), 8.19 (d, J=0.5 Hz, 1H), 7.79 (dd, J=8.6, 1.4 Hz, 1H), 7.64-7.53 (m, 3H), 4.30-3.42 (m, 11H).

LCMS (ESI-TOF) m/z 461.1 [M+H⁺] with a purity of >98%.

### (1-(Tert-butyl)-1H-1,2,3-triazol-4-yl)(4-(4-chloro-2-phenylquinoline-7-carbonyl)piperazin-1-yl)methanone (D008)

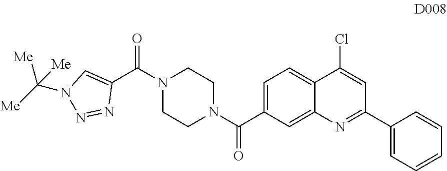 [SMILES: CC(C)(C)n1cc(C(=O)N2CCN(C(=O)c3ccc4c(Cl)cc(-c5ccccc5)nc4c3)CC2)nn1]

Step 1: (1-(Tert-butyl)-1H-1,2,3-triazol-4-yl)(piperazin-1-yl)methanone was synthesized according to General Procedure C1 using 1-(tert-butyl)-1H-1,2,3-trizole-4-carboxylic acid and tert-butyl piperazine-1-carboxylate as starting materials, followed by treatment with 1:1 trifluoroacetic acid/dichloromethane.

Step 2: Compound D008 was synthesized according to General Procedure C1 using intermediate from Step 1 and 4-chloro-2-phenylquinoline-7-carboxylic acid as starting materials.

¹H NMR (400 MHz, DMSO-d₆) δ 8.67 (s, 1H), 8.49 (s, 1H), 8.34 (dd, J=7.7, 1.6 Hz, 2H), 8.30 (d, J=8.5 Hz, 1H), 8.19 (s, 1H), 7.79 (dd, J=8.5, 1.3 Hz, 1H), 7.62-7.50 (m, 3H), 4.34-3.44 (m, 8H), 1.63 (s, 9H).

LCMS (ESI-TOF) m/z 503.2 [M+H⁺] with a purity of >99%.

### (4-Chloro-2-(4-(2-hydroxypropan-2-yl)phenyl)quinolin-7-yl)(4-(1-cyclopropyl-1H-1,2,3-triazole-4-carbonyl)piperazin-1-yl)methanone (D009)

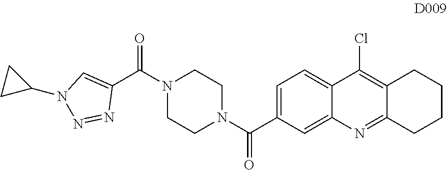 [SMILES: O=C(c1ccc2c(Cl)c3c(nc2c1)CCCC3)N1CCN(C(=O)c2cn(C3CC3)nn2)CC1]

Compound D009 was synthesized according to General Procedure C1 using (1-cyclopropyl-1H-1,2,3-triazol-4-yl)(piperazin-1-yl)methanone and commercially available 9-chloro-5,6,7,8-tetrahydroacridine-3-carboxylic acid as starting materials.

¹H NMR (400 MHz, DMSO-d₆) δ 8.60 (s, 1H), 8.20 (d, J=8.5 Hz, 1H), 7.98 (d, J=0.9 Hz, 1H), 7.68 (dd, J=8.7, 1.4 Hz, 1H), 3.87 (dd, J=210.0, 94.7 Hz, 9H), 3.06 (s, 2H), 2.99 (s, 2H), 1.90 (s, 4H), 1.18 (dd, J=39.4, 4.8 Hz, 4H).

LCMS (ESI-TOF) m/z 465.2 [M+H⁺] with a purity of >98%. (9-Chloro-6-(pyridin-2-yl)-5,6,7,8-tetrahydroacridin-3-yl)(4-(1-cyclopropyl-1H-1,2,3-triazole-4-carbonyl)piperazin-1-yl)methanone (D010)

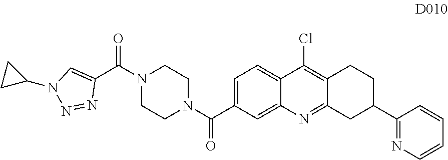 [SMILES: O=C(c1ccc2c(Cl)c3c(nc2c1)CC(c1ccccn1)CC3)N1CCN(C(=O)c2cn(C3CC3)nn2)CC1]

Compound D010 was prepared according to General Procedure A, B, and C1 using 3-(pyridin-3-yl)cyclohexanone (General Procedure A) and (1-cyclopropyl-1H-1,2,3-triazol-4-yl)(piperazin-1-yl)methanone (General Procedure C1) as starting materials.

¹H NMR (400 MHz, DMSO-d₆) δ 8.60 (s, 1H), 8.54 (d, J=3.9 Hz, 1H), 8.23 (d, J=8.4 Hz, 1H), 8.02 (d, J=1.0 Hz, 1H), 7.78 (td, J=7.7, 1.8 Hz, 1H), 7.70 (dd, J=8.6, 1.4 Hz, 1H), 7.43 (d, J=8.1 Hz, 1H), 7.32-7.23 (m, 1H), 3.87 (dd, J=220.9, 106.0 Hz, 10H), 3.06 (dd, J=32.5, 25.9 Hz, 4H), 2.27 (s, 1H), 2.10 (s, 1H), 1.31-1.01 (m, 4H).

LCMS (ESI-TOF) m/z 542.2 [M+H⁺] with a purity of >99%.

### (4-Chloro-2-phenylquinolin-7-yl)(4-(1-cyclopropyl-1H-pyrazole-4-carbonyl)piperazin-1-yl)nethanone (D011)

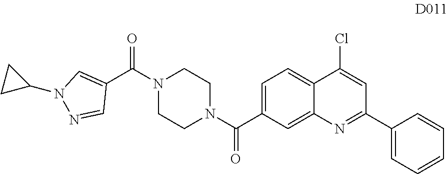 [SMILES: O=C(c1ccc2c(Cl)cc(-c3ccccc3)nc2c1)N1CCN(C(=O)c2cnn(C3CC3)c2)CC1]

Step 1: (1-Cyclopropyl-1H-pyrazol-4-yl)(piperazin-1-yl)methanone was synthesized according to General Procedure C1 using 1-cyclopropyl-1H-pyrazole-4-carboxylic acid and tert-butyl piperazine-1-carboxylate as starting materials, followed by treatment with 1:1 trifluoroacetic acid/dichloromethane.

Step 2: Compound D011 was synthesized according to General Procedure C1 using intermediate from Step 1 and 4-chloro-2-phenylquinoline-7-carboxylic acid as starting materials.

¹H NMR (400 MHz, DMSO-d₆) δ 8.49 (s, 1H), 8.34 (dd, J=7.8, 1.4 Hz, 2H), 8.30 (d, J=8.6 Hz, 1H), 8.17 (d, J=1.1 Hz, 1H), 8.14 (s, 1H), 7.78 (dd, J=8.6, 1.5 Hz, 1H), 7.68 (s, 1H), 7.62-7.51 (m, 3H), 3.81-3.38 (m, 9H), 1.14-0.87 (m, 4H).

LCMS (ESI-TOF) m/z 486.2 [M+H⁺] with a purity of >99%.

### (2-(4-(1-Amino-2-methylpropan-2-yl)phenyl)-4-chloroquinolin-7-yl)(4-(1-cyclopropyl-1H-1,2,3-triazole-4-carbonyl)piperazin-1-yl)nethanone (D012)

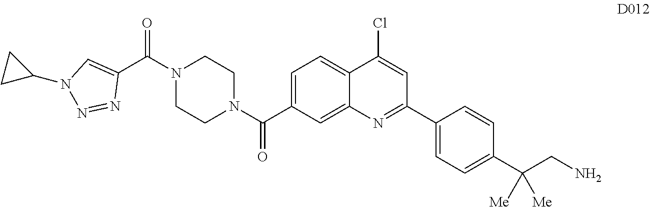 [SMILES: CC(C)(CN)c1ccc(-c2cc(Cl)c3ccc(C(=O)N4CCN(C(=O)c5cn(C6CC6)nn5)CC4)cc3n2)cc1]

Step 1: (1-Cyclopropyl-1H-1,2,3-triazol-4-yl) (4-(2,4-dichloroquinoline-7-carbonyl)piperazin-1-yl)methanone was synthesized according to General Procedure C1 using S9 (where R¹=H) and (1-cyclopropyl-1H-1,2,3-triazol-4-yl)(piperazin-1-yl)methanone as starting materials.

Step 2: Compound D012 was synthesized according to General Procedure L using the above intermediate and (4-(1-amino-2-methylpropan-2-yl)phenyl)boronic acid hydrochloride as starling materials.

¹H NMR (400 MHz, DMSO-d₆) δ 8.60 (s, 1H), 8.45 (s, 1H), 8.31-8.22 (m, 3H), 8.16 (d, J=1.1 Hz, 1H), 7.77 (dd, J=8.6, 1.5 Hz, 1H), 7.54 (d, J=8.5 Hz, 2H), 4.25-3.96 (m, 3H), 3.85-3.42 (m, 6H), 2.71 (s, 2H), 1.29 (s, 6H), 1.26-1.06 (m, 4H).

LCMS (ESI-TOF) m/z 558.2 [M+H⁺] with a purity of >99%.

### (2-(4-(1-Aminocyclopropyl)phenyl)-4-chloroquinolin-7-yl)(4-(1-cyclopropyl-1H-1,2,3-triazole-4-carbonyl)piperazin-1-yl)methanone (D013)

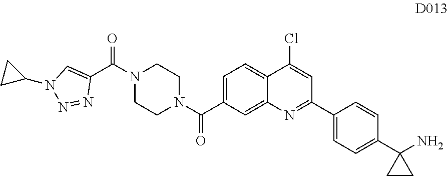 [SMILES: NC1(c2ccc(-c3cc(Cl)c4ccc(C(=O)N5CCN(C(=O)c6cn(C7CC7)nn6)CC5)cc4n3)cc2)CC1]

Compound D013 was synthesized according to General Procedure L using the above intermediate and 4-(1-aminocyclopropyl)phenylboronic acid hydrochloride as starting materials.

¹H NMR (400 MHz, DMSO-d₆) δ 8.60 (s, 1H), 8.46 (s, 1H), 8.31-8.20 (m, 3H), 8.15 (d, J=1.1 Hz, 1H), 7.75 (dd, J=8.5, 1.6 Hz, 1H), 7.48 (d, J=8.6 Hz, 2H), 4.25-3.95 (m, 3H), 3.88-3.45 (m, 6H), 1.27-1.10 (m, 4H), 1.10-0.97 (m, 4H).

LCMS (ESI-TOF) m/z 542.2 [M+H⁺] with a purity of >96%.

### (2-(3-(Aminomethyl)-4-methoxyphenyl)-4-chloroquinolin-7-yl)(4-(1-cyclopropyl-1H-1,2,3-triazole-4-carbonyl)piperazin-1-yl)methanone (D014)

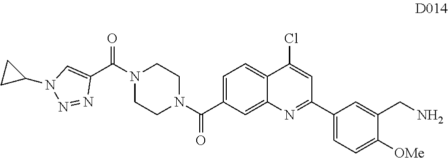 [SMILES: COc1ccc(-c2cc(Cl)c3ccc(C(=O)N4CCN(C(=O)c5cn(C6CC6)nn5)CC4)cc3n2)cc1CN]

Compound D014 was synthesized according to General Procedure L and then General Procedure D using 3-cyano-4-methoxyphenyl boronic acid as starting materials (General Procedure L).

¹H NMR (400 MHz, DMSO-d₆) δ 8.61 (s, 1H), 8.44 (s, 1H), 8.33 (d, J=2.2 Hz, 1H), 8.25 (d, J=8.6 Hz, 1H), 8.22 (dd, J=8.7, 2.4 Hz, 1H), 8.14 (d, J=1.1 Hz, 1H), 7.73 (dd, J=8.5, 1.6 Hz, 1H), 7.13 (d, J=8.7 Hz, 1H), 4.27-3.98 (m, 3H), 3.89 (s, 3H), 3.85-3.61 (m, 6H), 3.51 (br s, 2H), 1.30-1.07 (m, 4H).

LCMS (ESI-TOF) m/z 546.2 [M+H⁺] with a purity of >98%.

### Allyl 4-(4-chloro-2,3-dimethylquinoline-7-carbonyl)piperazine-1-carboxylate (C001)

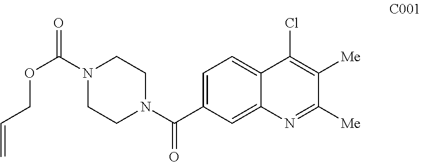 [SMILES: C=CCOC(=O)N1CCN(C(=O)c2ccc3c(Cl)c(C)c(C)nc3c2)CC1]

Compound C001 was prepared from General Procedure A, B and C2 using 2-butanone and 2-amino-terephthalic acid (General Procedure A) and allyl piperazine-1-carboxylate (General Procedure C2) as starting materials.

¹H NMR (400 MHz, DMSO-d₆) δ 8.19 (d, J=8.8 Hz, 1H), 7.96 (s, 1H), 7.67 (d, J=8.8 Hz, 1H), 5.94-5.92 (m, 1H), 5.31-5.18 (m, 2H), 4.55 (d, J=5.2 Hz, 2H), 3.71-3.32 (m, 8H), 2.71 (s, 3H), 2.55 (s, 3H).

LCMS (ESI-TOF) m/z 388.2 [M+H⁺] with a purity of >96%.

### Allyl 4-(9-chloro-2,3-dihydro-1H-cyclopenta[b]quinoline-6-carbonyl)piperazine-1-carboxylate (C002)

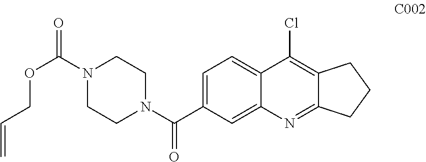 [SMILES: C=CCOC(=O)N1CCN(C(=O)c2ccc3c(Cl)c4c(nc3c2)CCC4)CC1]

Compound C002 was prepared using cyclopentanone and 2-amino-terephthalic acid as starting materials for cyclization using conditions similar to General Procedure A and B. The resulting product was reacted with allyl piperazine-1-carboxylate according to General Procedure C2.

¹H NMR (400 MHz, DMSO-d₆) δ 8.18 (d, J=8.8 Hz, 1H), 7.99 (d, J=1.2 Hz, 1H), 7.67 (dd, J=8.4, 1.6 Hz, 1H), 5.96-5.89 (m, 1H), 5.31-5.18 (m, 2H), 4.55 (d, J=4.8 Hz, 2H), 3.68-3.41 (m, 8H), 3.19-3.13(m, 4H), 2.23-2.16 (m, 2H).

LCMS (ESI-TOF) m/z 400.2 [M+H⁺] with a purity of >96%.

Propyl 4-(4-chloro-2,3-dimethylquinoline-7-carbonyl)piperazine-1-carboxylate (C003)

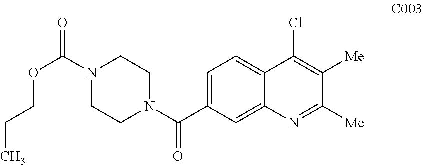 [SMILES: CCCOC(=O)N1CCN(C(=O)c2ccc3c(Cl)c(C)c(C)nc3c2)CC1]

Compound C003 was prepared from General Procedure A, B and C2 using 2-butanone and 2-amino-terephthalic acid (General Procedure A) and n-propyl piperazine-1-carboxylate (General Procedure C2) as starting materials.

¹H NMR (400 MHz, DMSO-d₆) δ 8.18 (d, J=8.8 Hz, 1H), 7.95 (s, 1H), 7.66 (d, J=0.8 Hz, 1H), 3.98 (d, J=6.8 Hz, 2H), 3.71-3.32 (m, 8H), 2.70 (s, 3H), 2.55 (s, 3H), 1.61-1.55 (m, 2H), 0.89 (t, J=6.8 Hz, 3H).

LCMS (ESI-TOF) m/z 390.4 [M+H⁺] with a purity of >99%.

### Propyl 4-(4-chloro-2-ethylquinoline-7-carbonyl)piperazine-1-carboxylate (C004)

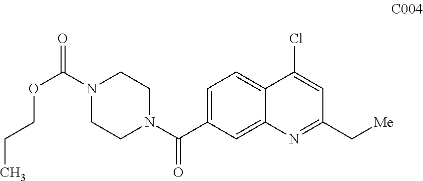 [SMILES: CCCOC(=O)N1CCN(C(=O)c2ccc3c(Cl)cc(CC)nc3c2)CC1]

Step 1: A mixture of 3-amino benzoic acid (5.0 g, 36.4 mmol) and methyl propionylacetate (30 mL) was heated to 90° C. and stirred for 24 h. After completion, the reaction mixture was washed with pentane to afford the resulting 3-(1-methoxy-1-oxopentan-3-ylideneamino)benzoic acid as a pale brown solid.

Step 2: A mixture of above imine (12.0 g, 48.1 mmol) and diphenyl ether (10 mL/g) was heated to 300° C. for 5 h in a sealed tube. The reaction mass was cooled to room temperature and diluted with hexanes. The resultant solid was collected by filtration and washed with hexanes to afford a mixture of desired methyl 2-ethyl-4-oxo-1,4-dihydroquinoline-7-carboxylate and its regioisomer as a brown gum.

Step 3: The above mixture (2.0 g, 8.65 mmol) and phosphorus oxychloride (5 mL/g) was heated at 100° C. for 4 h. The reaction mass was concentrated under reduced pressure and excess of cold water was added to the residue, stirred until a free solid was formed. The resultant solid was collected by filtration, washed with hexane and dried. The crude product was purified by column chromatography (ethyl acetate/petroleum ether) to afford methyl 4-chloro-2-ethylquinoline-7carboxylate as an off-white solid.

Step 4: To a well stirred solution of the above intermediate (200 mg, 0.808 mmol) in a mixture of methanol, tetrahydrofuran and water (1:1:0.5 mL) was added lithium hydroxide monohydrate (136 mg, 3.23 mmol) and the reaction mixture was stirred at room temperature for 2 h. The reaction mixture was concentrated under reduced pressure. The residue was taken in cold water and acidified with 6 M hydrochloric acid, and the precipitated solid was collected by filtration to afford 4-chloro-2-ethylquinoline-7-carboxylic acid as brown solid.

Step 5: The above acid was reacted with n-propyl piperazine-1-carboxylate according to General Procedure C2 to afford C004.

¹H NMR (400 MHz, DMSO-d₆) δ 8.22 (d, J=8.4 Hz, 1H), 8.01 (d, J=1.2 Hz, 1H), 7.80 (s, 1H), 7.69 (dd, J=1.6, 8.4 Hz, 1H), 3.97 (t, J=6.6 Hz, 2H), 3.72-3.32 (m, 8H), 2.99-2.93 (m, 2H), 1.61-1.55 (m, 2H), 1.32 (t, J=7.6 Hz, 3H), 0.89 (t, J=7.2 Hz, 3H).

LCMS (ESI-TOF) m/z 390.2 [M+H⁺] with a purity of >99%.

### Propyl 4-(4-chloro-3-ethylquinoline-7-carbonyl)piperazine-1-carboxylate (C005)

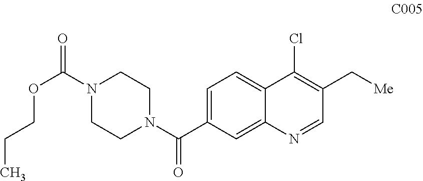 [SMILES: CCCOC(=O)N1CCN(C(=O)c2ccc3c(Cl)c(CC)cnc3c2)CC1]

Step 1: To a suspension of 3-aminobenzoic acid (1 g, 7.3 mmol) in ethanol (15 mL) was added diethyl 2-ethyl-3-oxosuccinate (1.58 g, 7.3 mmol) and the mixture was stirred at reflux temperature. After 72 h, another portion of diethyl 2-ethyl-3-oxosuccinate (0.79 g, 3.65 mmol) was added and the reflux was continued for another 24 h. The reaction mass was concentrated under reduced pressure and the residue was stirred with a solution of 10% methanol in dichloromethane. The solids were separated by filtration and the filtrate was concentrated to afford 3-(1-Ethoxy-3-(ethoxycarbonyl)-1-oxopent-2-en-2-ylamino)benzoic acid as a brown colored solid.

Step 2: To a pre-heated dodecylbenzene (10 mL) was added the above intermediate (1 g , 2.98 mmol) at 250° C. The resulting mixture was stirred at same temperature for 6 h and then high vacuum was applied for 5 min. The reaction mass was cooled to room temperature and diluted with hexanes (50 mL) and stirred for 10 min. The filtrate was separated from the residue and the residue was purified by flash chromatography followed by preparative-HPLC to afford ethyl 3-ethyl-4-oxo-1,4-dihydroquinoline-7-carboxylate as a brown solid.

Step 3: To a stirred solution of the above intermediate (50 mg, 0.20 mmol) in tetrahydrofuran/water (2:1, 10 mL) was added lithium hydroxide monohydrate (25.7 mg, 0.61 mmol) and stirred at room temperature for 20 h. The reaction mass was concentrated and the residue was dissolved in cold water (5 mL) and the resulting solution was acidified with 2 M hydrochloric acid to a pH 4. The resultant solid was collected by filtration and washed with cold water, hexanes and dried to afford ethyl-4-oxo-1,4-dihydroquinoline-7-carboxylic acid as an off-white solid.

Step 4: A mixture of the above acid (40 mg, 0.18 mmol) and phosphorus oxychloride (1 mL) was stirred at 100° C. After 4 h, the reaction mass was concentrated under reduced pressure, and cold water (10 mL) was added to the residue. The resultant solids were collected by filtration and washed with cold water, hexanes and dried to afford 4-chloro-3-ethylquinoline-7-carboxylic acid as an off white solid.

Step 5: The above acid was reacted with n-propyl piperazine-1-carboxylate according to General Procedure C2 to afford C005.

¹H NMR (400 MHz, DMSO-d₆) δ 8.95 (s, 1H), 8.28 (d, J=8.4 Hz, 1H), 8.07 (d, J=1.6 Hz, 1H), 7.76 (dd, J=2.0, 8.8 Hz, 1H), 3.97 (t, J=7.2 Hz, 2H), 3.7-3.32 (m, 8H), 2.97 (q, J=7.2 Hz, 2H), 1.62-1.54 (m, 2H), 1.28 (t, J=7.6 Hz, 3H), 0.89 (t, J=7.6 Hz, 3H).

LCMS (ESI-TOF) m/z 390.3 [M+H⁺] with a purity of >98%.

### Propyl 4-(4-chloro-3-cyano-2-methylquinoline-7-carbonyl)piperazine-1-carboxylate (C006)

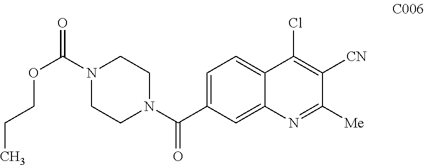 [SMILES: CCCOC(=O)N1CCN(C(=O)c2ccc3c(Cl)c(C#N)c(C)nc3c2)CC1]

Compound C006 was prepared using 2-aminoterphthalic acid and 3-oxobutyronitrile as reagents for quinoline synthesis. The conditions were similar to that reported in General Procedure I. The resulting intermediate was reacted with n-propyl piperazine-1-carboxylate using General Procedure C2 conditions.

¹H NMR (400 MHz, DMSO-d₆) δ 8.30 (d, J=8.8 Hz, 1H), 8.06 (d, J=1.6 Hz, 1H), 7.80 (dd, J=1.6, 8.4 Hz, 1H), 3.95 (t, J=6.6 Hz, 2H), 3.70-3.25 (m, 8H), 2.83 (s, 3H), 1.60-1.50 (m, 2H), 0.89 (t, J=7.2 Hz, 3H).

LCMS (ESI-TOF) m/z 401.2 [M+H⁺] with a purity of >99%.

### Propyl 3-(4-chloroquinoline-7-carbonyl)-3,9-diazabicyclo[3.3.1]nonane-9-carboxylate (C007)

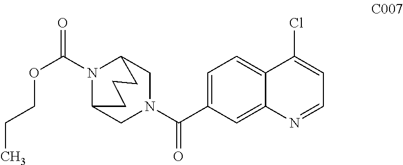 [SMILES: CCCOC(=O)N1C2CCCC1CN(C(=O)c1ccc3c(Cl)ccnc3c1)C2]

Step 1: According to General Procedure C1, commercially available 4-chloroquinoline-7-carboxylic acid was reacted with tert-butyl 7,9-diazabicyclo[3.3.1]nonane-9-carboxylate to give tert-butyl 3-(4-chloroquinoline-7-carbonyl)-3,9-diazabicyclo [3.3.1]nonane-9-carboxylate.

Step 2: To a solution of the intermediate from above (91.5 mg, 0.22 mmol) in dichloromethane (0.8 mL) was added trifluoroacetic acid (0.37 mL, 4.835 mmol, 22 equiv). The resulting mixture was stirred for 4 h before concentrating under reduced pressure. The residue was dissolved in ethyl acetate and then washed with saturated sodium bicarbonate. The organic layer was dried over anhydrous sodium sulfate, filtered and concentrated under reduced pressure to give (3,9-diazabicyclo[3.3.1]nonan-3-yl) (4-chloroquinolin-7-yl)methanone.

Step 3: To a solution of the above residue (48.2 mg, 0.153 mmol) in dichloromethane (1.5 mL) was added triethylamine (0.043 mL, 0.308 mmol, 2 equiv) and propyl chloroformate (0.030 mL, 0.267 mmol, 1.7 equiv). The mixture was stirred for 30 min before quenching by the addition of saturated sodium bicarbonate. The aqueous layer was extracted 3 times with ethyl acetate and the combined organics were dried over anhydrous sodium sulfate, filtered and concentrated under reduced pressure. The crude material was purified by column chromatography (ethyl acetate/hexanes) to afford C007 as a white solid (29 mg, 47%) upon lyophilization.

¹H NMR (400 MHz, DMSO-d₆) δ 8.92 (d, J=4.7 Hz, 1H), 8.32 (d, J=8.6 Hz, 1H), 8.05 (s, 1H), 7.86 (d, J=4.7 Hz, 1H), 7.75 (d, J=8.6 Hz, 1H), 4.62 (d, J=13.3 Hz, 1H), 4.25 (s, 1H), 4.02 (br s, 2H), 3.62-3.45 (m, 2H), 3.14-3.11 (m, 1H), 2.12-2.02 (m, 2H), 1.86-1.50 (m, 7H), 0.93-0.85 (m, 3H).

LCMS (ESI-TOF) m/z 402.1 [M+H⁺] with a purity of >97%.

### Propyl 4-(10-chloro-1,2,3,4-tetrahydrobenzo[b][1,6]naphthyridine-7-carbonyl)piperazine-1-carboxylate (C008)

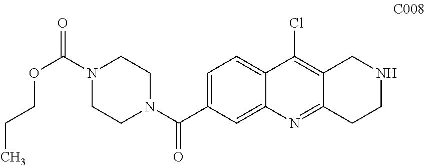 [SMILES: CCCOC(=O)N1CCN(C(=O)c2ccc3c(Cl)c4c(nc3c2)CCNC4)CC1]

Compound C008 was prepared using 2-aminoterphthalic acid and 1-benzylpiperidin-4-one as starting materials for quinoline synthesis using similar conditions to General Procedure A and B. The resulting intermediate was reacted with n-propyl piperazine-1-carboxylate according to General Procedure C2. The resulting intermediate (0.7 g, 1.38 mmol) was dissolved in 1,2-dichloroethane (10 mL) and chloroethyl chloroformate (130.3 mg, 0.91 mmol) was added at 0° C. The reaction mixture was then stirred at 80° C. for 2 h. Upon cooling, the reaction mixture was concentrated under reduced pressure. To the resulting residue was added methanol (40 mL) and the mixture was stirred at 60° C. for 1 h. The reaction mixture was concentrated under reduced pressure and drops of concentrated hydrochloric acid were added and then partitioned between ethyl acetate and saturated sodium carbonate solution. The combined organic layer was washed with water and brine, dried over anhydrous sodium sulfate, filtered, concentrated. The crude material was purified by preparative-HPLC to afford C008.

¹H NMR (400 MHz, CDC1₃) δ 8.25 (d, J=8.0 Hz, 1H), 7.99 (s, 1H), 7.61 (dd, J=8.8, 1.6 Hz, 1H), 4.29 (s, 2H), 4.08 (t, J=6.4 Hz, 2H), 3.82-3.48 (m, 8H), 3.33-3.30 (m, 2H), 3.18-3.15 (m, 2H), 1.69-1.64 (m, 3H), 0.95 (t, J=7.6 Hz, 3H).

LCMS (ESI-TOF) m/z 417.2 [M+H⁺] with a purity of >95%.

### (2-(3-(Aminomethyl)-4-fluorophenyl)-4-chloroquinolin-7-yl)(4-(1-cyclopropyl-1H-1,2,3-triazole-4-carbonyl)piperazin-1-yl)methanone (E019)

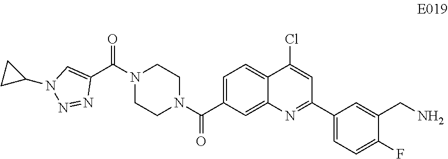 [SMILES: NCc1cc(-c2cc(Cl)c3ccc(C(=O)N4CCN(C(=O)c5cn(C6CC6)nn5)CC4)cc3n2)ccc1F]

Compound E019 was synthesized according to General Procedure L by using [2-fluoro-5-(tetramethyl-1,3,2-dioxaborolan-2-yl)phenyl]methanamine hydrochloride as starting material.

¹H NMR (400 MHz, DMSO-d₆) δ 8.61 (s, 1H), 8.50 (s, 1H), 8.47 (dd, J=7.4, 2.2 Hz, 1H), 8.29 (d, J=8.5 Hz, 1H), 8.27-8.22 (m, 1H), 8.18 (d, J=1.2 Hz, 1H), 7.78 (dd, J=8.6, 1.5 Hz, 1H), 7.33 (dd, J=9.6, 8.8 Hz, 1H), 4.25-3.96 (m, 3H), 3.86 (s, 2H), 3.84-3.60 (m, 4H), 3.51 (s, 2H), 2.02 (s, 1H), 1.27-1.09 (m, 4H).

LCMS (ESI-TOF) m/z 534.2 [M+H⁺] with a purity of >99%.

### (4-Chloro-2-(3-((dimethylamino)methyl)-4-fluorophenyl)quinolin-7-yl)(4-(1-cyclopropyl-1H-1,2,3-triazole-4-carbonyl)piperazin-1-yl)methanone (E020)

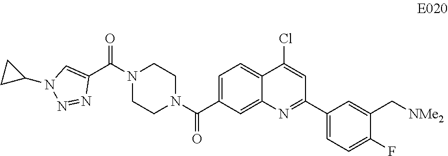 [SMILES: CN(C)Cc1cc(-c2cc(Cl)c3ccc(C(=O)N4CCN(C(=O)c5cn(C6CC6)nn5)CC4)cc3n2)ccc1F]

Compound E020 was synthesized according to General Procedure L by using 3-((dimethylamino) methyl)-4-fluorophenylboronic acid hydrochloride as starting material.

¹H NMR (400 MHz, DMSO-d₆) δ 8.60 (s, 1H), 8.49 (s, 1H), 8.38 (dd, J=7.2, 2.3 Hz, 1H), 8.32-8.25 (m, 2H), 8.19 (s, 1H), 7.78 (dd, J=8.6, 1.5 Hz, 1H), 7.37 (dd, J=9.4, 9.0 Hz, 1H), 4.28-3.95 (m, 3H), 3.85-3.61 (m, 4H), 3.61-3.42 (m, 4H), 2.22 (s, 6H), 1.18 (d, J=36.4 Hz, 4H).

LCMS (ESI-TOF) m/z 562.2 [M+H⁺] with a purity of >98%.

### Comparative Example 1

The following compounds in Table 5 are disclosed in the prior art and were synthesized and tested.

TABLE 4
Table showing the list of prior art compounds and their biological activity

| Compound | | IC50 |
|---|---|---|
| name | Structure | (μm) |
| X1 | 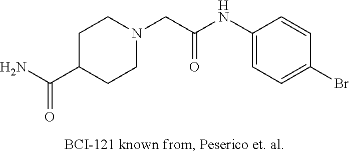 [SMILES: NC(=O)C1CCN(CC(=O)Nc2ccc(Br)cc2)CC1] | >250 |
| X2 | 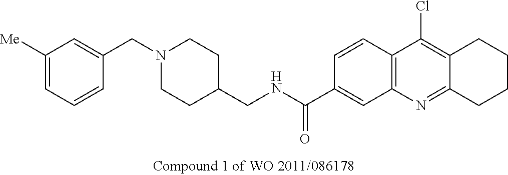 [SMILES: Cc1cccc(CN2CCC(CNC(=O)c3ccc4c(Cl)c5c(nc4c3)CCCC5)CC2)c1] | 750 |
| X3 | 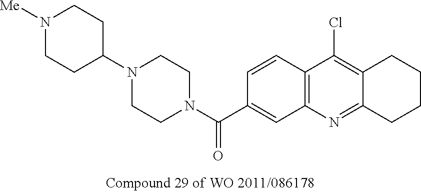 [SMILES: CN1CCC(N2CCN(C(=O)c3ccc4c(Cl)c5c(nc4c3)CCCC5)CC2)CC1] | 750 |
| X4 | 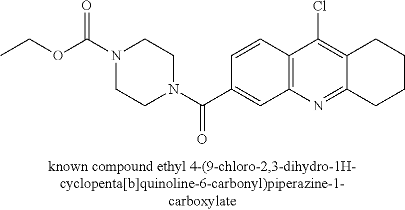 [SMILES: CCOC(=O)N1CCN(C(=O)c2ccc3c(Cl)c4c(nc3c2)CCCC4)CC1] | 6.2 |
| X5 | 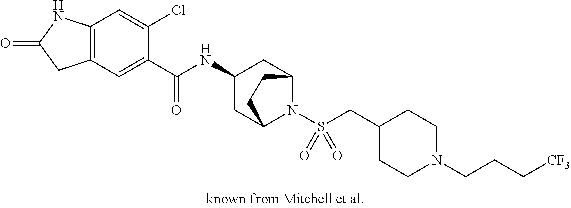 [SMILES: O=C1Cc2cc(C(=O)N[C@H]3C[C@H]4CC[C@@H](C3)N4S(=O)(=O)CC3CCN(CCCC(F)(F)F)CC3)c(Cl)cc2N1] | ND |

There was no reported biochemical assay data in Peserico et.al. for comparative compound X 1 , and comparative compound X1 was found to be inactive against SMYD3.

Comparative compound X2 and X3 were found to act against a different target ubiquitin specific protease 7, but were not active against SMYD3.

Comparative compound X4 was found to be moderately active against SMYD3 (6.2 μM), but suffered from poor metabolic stability due to high metabolic clearance in the human/mouse liver microsomes stability tests (half-life, t_{1/2}=9 min/4 min respectively). In addition, comparative compound X4 was found to have poor target engagement compared to a more advanced compound B019.

Comparative compound XS was found to be very active against SMYD3 (3 nM) using the specified assay reported in the publication. Although the reported molecule was active against SMYD3, no anti-proliferative cellular activity was disclosed. Moreover, the structure of the inhibitor is not related to the compounds in this application.

### INDUSTRIAL APPLICABILITY

The compounds as defined above may find a multiple number of applications in which their ability to inhibit protein lysine methyltransferases such as SMYD3. The compounds may also be used in treating or preventing a condition or disorder in a mammal in which inhibition of a protein methyl transferase and/or co-factor thereof and/or via an unspecified mechanism prevents, inhibits or ameliorates apathology or a symptomology of the condition. The condition or disorder may be cancer, angiogenic disorder or pathological angiogenesis, fibrosis and inflammatory conditions. The compounds may be particularly useful in treating cancer such as breast, gastric, pancreatic, colorectal, lung cancer and hepatocellular carcinoma and other hypervascular tumors as well as angiogenic diseases.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A compound having the following Formula (I); 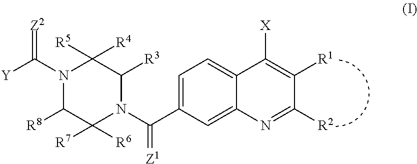
wherein
Z¹ and Z² are independently selected from O, S or NH;
X is a halogen;
R¹ and R² are independently selected from the group consisting of a bond, H, halogen, cyano, optionally substituted alkyl, optionally substituted alkene, optionally substituted alkyne, optionally substituted alkoxy, optionally substituted amino, optionally substituted cycloalkyl, optionally substituted heterocycloalkyl, optionally substituted aryl and optionally substituted heteroaryl;
and wherein R¹ and R² may optionally be taken together to form an optionally substituted alkylene bridge or an optionally substituted alkylene bridge wherein one or two alkylene units may be replaced with O, NH or S;
and wherein R¹ and R² may optionally form an optionally substituted aryl or optionally substituted heteroaryl together with the ring atoms that they are bonded to;
R³, R⁴, R⁵, R⁶, R⁷ and R⁸ areindependently absent, or selected from the group consisting of a bond, H, halogen, optionally substituted alkyl, optionally substituted alkene, optionally substituted alkyne, optionally substituted alkoxy, optionally substituted amino, optionally substituted acyl, optionally substituted cycloalkyl, optionally substituted heterocycloalkyl, optionally substituted aryl and optionally substituted heteroaryl;
wherein any two of R³, R⁴, R⁵, R⁶, R⁷ or R⁸ may be taken together to form an optionally substituted cycloalkyl, an optionally substituted alkylene bridge or an optionally substituted alkylene bridge wherein one or two alkylene units may be replaced with O, NH or S;
Y is selected from R⁹, OR⁹ or NHR⁹, wherein R⁹ is an optionally substituted C₃ to C₁₀ alkyl, optionally substituted C₃ to C₁₀ alkenyl, optionally substituted C₃ to C₁₀ alkynyl, optionally substituted C₃ to C₇ cycloalkyl, optionally substituted C₂ to C₁₀ haloalkyl, a substituted 5-membered heteroaryl comprising two to three heteroatoms selected from N, O or S or a C₁ to C₂ alkyl substituted with an optionally substituted 5-membered heterocycloalkyl comprising one to two heteroatoms selected from N, O or S;
or a pharmaceutically acceptable form or prodrug thereof.

2.-4. (canceled)

5. The compound according to claim 1, having the following Formula (II):

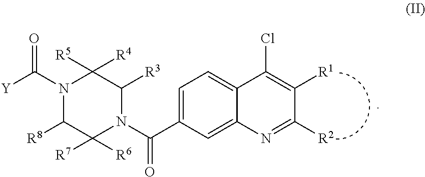

6. (canceled)

7. The compound according to claim 1, wherein R⁹ is selected from a C₃ to C₁₀ alkyl, a C₃ to C₁₀ alkenyl, C₂ to C₁₀ haloalkyl, or a in each case C₃ to C₉ alkyl or C₃ to C₇ cycloalkyl substituted oxazolyl, isoxazolyl, 1,2-azole, pyrazolyl, triazolyl, or methylpyrrolidinonyl.

8. The compound according to claim 1, having the following formula (IIa): 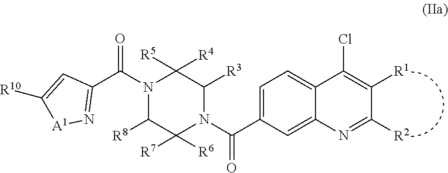
wherein A¹ is O or NH, and
R¹⁰ is a C₁ to C₉ alkyl or a C₃ to C₇ cycloalkyl.

9. (canceled)

10. The compound according to claim 1, wherein R¹ and R² are independently selected from the group consisting of a bond, H, halogen, cyano, optionally substituted alkyl, optionally substituted phenyl, optionally substituted pyrazolyl, optionally substituted thiazolyl, optionally substituted thiophenyl, optionally substituted benzo[d]imidazolyl, optionally substituted indolyl, optionally substituted isoindolyl, optionally substituted indazolyl, optionally substituted pyrrolyl, optionally substituted pyridinyl, optionally substituted benzyl, optionally substituted benzo[d]dioxolyl, optionally substituted benzotriazolyl, optionally substituted benzoxazolyl, optionally substituted benzofuranyl, optionally substituted pyrazolopyridinyl, optionally substituted pyrrolopyrimidinyl, optionally substituted pyrrolopyridinyl, optionally substituted naphthyridinyl, optionally substituted pyrimidinyl, optionally substituted benzothiazolyl, optionally substituted cyclopropyl, an amino group optionally substituted with an optionally substituted phenyl and an amino group optionally substituted with an optionally substituted pyridinyl.

11. The compound according to claim 1, having the following formula (IIb):

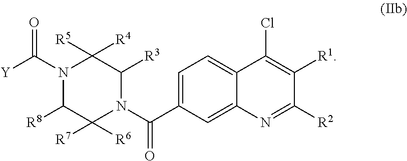

12. The compound according to claim 11, wherein R¹ is H or halogen, and R² is selected from the group consisting of H, cyano, methyl, ethyl, ethynyl, phenyl, 2-methylphenyl, 3-methylphenyl, 4-methylphenyl, 2-hydroxyphenyl, 3-hydroxyphenyl, 4-hydroxyphenyl, 2-(trifluoromethyl)phenyl, 3-(trifluoromethyl)phenyl, 4-(trifluoromethyl)phenyl, 2-chlorophenyl, 3-chlorophenyl, 4-chlorophenyl, 2-fluorophenyl, 3-fluorophenyl, 4-fluorophenyl, 2-fluoromethylphenyl, 3-fluoromethylphenyl, 4-fluoromethylphenyl, 2-hydroxymethylphenyl, 3-hydroxymethylphenyl, 4-hydroxymethylphenyl, 2-methoxyphenyl, 3-methoxyphenyl, 4-methoxyphenyl, 2-ethoxyethylphenyl, 3-ethoxyethylphenyl, 4-ethoxyethylphenyl, 2-(azidomethyl)phenyl, 3-(azidomethyl)phenyl, 4-(azidomethyl)phenyl, 2,3-difluorophenyl, 2,4-difluorophenyl, 2,5-difluorophenyl, 2,6-difluorophenyl, 3,4-difluorophenyl, 3,5-difluorophenyl, 3,5-difluoro-4-hydroxyphenyl, 3,5-difluoro-4-(aminocarbonyl)phenyl, 3,5-difluoro-4-aminomethylphenyl, 2-cyanophenyl, 3-cyanophenyl, 4-cyanophenyl, 2-(cyanomethyl)phenyl, 3-(cyanomethyl)phenyl, 4-(cyanomethyl)phenyl, 2-nitrophenyl, 3-nitrophenyl, 4-nitrophenyl, 2-aminophenyl, 3-aminophenyl, 4-aminophenyl, 2-(aminomethyl)phenyl, 3-(aminomethyl)phenyl, 4-(aminomethyl)phenyl, 2-(dimethylamino)phenyl, 3-(dimethylamino)phenyl, 4-(dimethylamino)phenyl, 2-(aminocarbonyl)phenyl, 3-(aminocarbonyl)phenyl, 4-(aminocarbonyl)phenyl, 2-(methylaminocarbonyl)phenyl, 3-(methylaminocarbonyl)phenyl, 4-(methylaminocarbonyl)phenyl, 2-(ethylaminocarbonyl)phenyl, 3-(ethylaminocarbonyl)phenyl, 4-(ethylaminocarbonyl)phenyl, 4-(1-ethoxyethyl)phenyl, 4-(2-hydroxy-2-propyl)phenyl, 2-pyridinyl, 3-pyridinyl, 4-pyridinyl, 2-methyl-3-pyridinyl, 4-methyl-3-pyridinyl, 5-methyl-3-pyridinyl, 6-methyl-3-pyridinyl, 6-methoxycarbonyl-3-pyridinyl, 2-thiophenyl, 3-thiophenyl, 1-pyrrolidinyl, 2-pyrrolidinyl, 3-pyrrolidinyl, 1-pyrrolyl, 2-pyrrolyl, 3-pyrrolyl, 1-methyl-3-pyrrolyl, 3-(1,2,5-trimethyl)-pyrrolyl, 2-ethynylphenyl, 3-ethynylphenyl, 4-ethynylphenyl, 2-ethylphenyl, 3-ethylphenyl, 4-ethylphenyl, 2-(1-hydroxyethyl)phenyl, 3-(1-hydroxyethyl) phenyl, 4-(1-hydroxyethyl)phenyl, 2-(2-hydroxyethyl) phenyl, 3-(2-hydroxyethyl)phenyl, 4-(2-hydroxyethyl)phenyl, 4-fluoro-3-methylphenyl, 4-fluoro-2-methylphenyl, 3-fluoro-2-methylphenyl, 3-fluoro-4-methylphenyl, 3-fluoro-5-methylphenyl, 2-fluoro-5-methylphenyl, 4-fluoro-3-methoxyphenyl, 4-fluoro-2-methoxyphenyl, 3-fluoro-2-methoxyphenyl, 3-fluoro-4-methoxyphenyl, 3-fluoro-5-methoxyphenyl, 2-fluoro-5-methoxyphenyl, 4-fluoro-3-hydroxyphenyl, 4-fluoro-2-hydroxyphenyl, 4-hydroxy-3-fluorophenyl, 4-hydroxy-2-fluorophenyl, 4-fluoro-3-hydroxymethylphenyl, 4-fluoro-2-hydroxymethylphenyl, 3-fluoro-2-hydroxymethylphenyl, 3-fluoro-4-hydroxymethylphenyl, 3-fluoro-5-hydroxymethylphenyl, 2-fluoro-5-hydroxymethylphenyl, 3-fluoro-4-(2-hydroxy-2-propyl)phenyl, 3-(aminocarbonyl)-4-fluorophenyl, 2-(aminocarbonyl)-4-fluorophenyl, 2-(aminocarbonyl)-3-fluorophenyl, 4-(aminocarbonyl)-3-fluorophenyl, 5-(aminocarbonyl)-3-fluorophenyl, 5-(aminocarbonyl)-2-fluorophenyl, 4-fluoro-3-(methylaminocarbonyl)phenyl, 3-fluoro-4-(methylaminocarbonyl)phenyl, 4-fluoro-2-(methylaminocarbonyl)phenyl, 3-fluoro-2-(methylaminocarbonyl)phenyl, 4-(cyclopropylaminocarbonyl)phenyl, 2-(cyclopropylaminocarbonyl)phenyl, 3-(cyclopropylaminocarbonyl)phenyl, 4-fluoro-3-(cyclopropylaminocarbonyl)phenyl, 3-fluoro-4-(cyclopropylaminocarbonyl)phenyl, 4-fluoro-2-(cyclopropylaminocarbonyl)phenyl, 3-fluoro-2-(cyclopropylaminocarbonyl)phenyl, (3-fluoro-4-(dimethylaminocarbonyl)phenyl, 3-fluoro-5-(dimethylaminocarbonyl)phenyl, 2-fluoro-5-(dimethylaminocarbonyl)phenyl, 4-fluoro-3-(dimethylaminocarbonyl)phenyl, 4-fluoro-2-(dimethylaminocarbonyl)phenyl, 3-fluoro-2-(dimethylaminocarbonyl)phenyl, 3-methyl-4-(methylaminocarbonyl)phenyl, 3-amino-4-fluorophenyl, 2-amino-4-fluorophenyl, 3-aminomethyl-4-fluorophenyl, 2-aminomethyl-4-fluorophenyl, 3-hydroxymethyl-4-methylphenyl, 2-hydroxymethyl-4-methyl-phenyl, 2-hydroxymethyl-3-methyl-phenyl, 4-hydroxymethyl-3-methylphenyl, 5-hydroxymethyl-3-methylphenyl, 5-hydroxymethyl-2-methylphenyl, 2-morpholinophenyl, 3-morpholinophenyl, 4-morpholinophenyl, 2-(pyrrolidin-1-yl)phenyl, 3-(pyrrolidin-1-yl)phenyl, 4-(pyrrolidin-1-yl)phenyl, 4-(1-amino-1-cyclopropyl)phenyl, cyclopropyl, cyclobutyl, cyclopentyl, cyclohexyl, 2-thiazolyl, 4-thiazolyl, 5-thiazolyl, 2-methylthiazolyl, 4-methylthiazolyl, 4-(dimethylamido)phenyl, 2-(dimethylamido)phenyl, 3-(dimethylamido)phenyl, 2-benzylamin, 3-benzylamin, 4-benzylamin, 2-methylaminophenyl, 3-methylaminophenyl, 4-methylaminophenyl, 6-(1-methyl)indazolyl, 6-(2-methyl)indazolyl, 5-(1-methyl)indazolyl, 4-(1-methyl)indazolyl, 3-(1-methyl)indazolyl, 5-(2-methyl)indazolyl, 5-(3-methyl)indazolyl, 5-(1-methyl)pyrazolyl, 4-(1-methyl)-pyrazolyl, 3-(1-methyl) pyrazolyl, 4-(1-isopropyl)-pyrazolyl, 4-(1-difluoromethyl)-pyrazolyl, 4-(5-trifluoromethyl)-pyrazolyl, 4-(1-(2,2,2)-trifluoroethyl)pyrazolyl, 4-(1-cyclopentyl)pyrazolyl, 2-(1-methyl) pyrazolyl-phenyl, 3-(1-methyl) pyrazolyl-phenyl, 4-(imidazol-1-yl)phenyl, 1-imidazolyl, 2-imidazolyl, 3-imidazolyl, 4-(4-methylpiperazino)phenyl, 3-(4-methylpiperazino)phenyl, 2-(4-methylpiperazino)phenyl, 3-[1,2,4]-triazol-4-ylphenyl, 2-[1,2,4]-triazol-4-yl phenyl, 4-[1,2,4]-triazol-4-ylphenyl, 3-(aminomethyl)-4-methoxyphenyl, 3-(aminomethyl)-5-methoxyphenyl, 2-(aminomethyl)-4-methoxyphenyl, 2-(aminomethyl)-5-methoxyphenyl, 2-(aminomethyl)-6-methoxyphenyl, 4-(aminomethyl)-3-methoxyphenyl, 2-(aminomethyl)-3-methoxyphenyl, 4-(dimethylaminomethyl)phenyl, 3-(dimethylaminomethyl)phenyl, 2-(dimethylaminomethyl)phenyl, 4-fluoro-3-(dimethylaminomethyl)phenyl, 4-fluoro-2-(dimethylaminomethyl)phenyl, 4-methoxy-3-methylphenyl, 2-methoxy-4-methylphenyl, 3-methoxy-5-methylphenyl, 3-methoxy-4-methylphenyl, 2-methoxy-5-methylphenyl, 2-methoxy-6-methylphenyl, 2-methoxy-3-methylphenyl, 4-methoxy-3-hydroxymethylphenyl, 3-methoxy-4-hydroxymethylphenyl, 2-methoxy-4-hydroxymethylphenyl, 3-methoxy-5-hydroxymethylphenyl, 2-methoxy-5-hydroxymethylphenyl, 2-methoxy-6-hydroxymethylphenyl, 2-methoxy-3-hydroxymethylphenyl, 4-hydroxy-3-hydroxymethylphenyl, 4-hydroxy-3-methylphenyl, 3-ethoxy-4-hydroxyphenyl, 3-hydroxy-4-methylphenyl, 2-hydroxy-4-methylphenyl, 3-cyano-4-methylphenyl, 4-cyano-3-methylphenyl, 2-cyano-4-methylphenyl, 3-cyano-5-methylphenyl, 2-cyano-5-methylphenyl, 2-cyano-6-methylphenyl, 2-cyano-3-methylphenyl, 4-(aminosulfonyl)phenyl, 3-(aminosulfonyl)phenyl, 2-(aminosulfonyl)phenyl, 3-(N,N-dimethylaminomethyl)-4-methoxyphenyl, 3-(N,N-dimethylaminomethyl)-5-methoxyphenyl, 2-(N,N-dimethylaminomethyl)-4-methoxyphenyl, 2-(N,N-dimethylaminomethyl)-5-methoxyphenyl, 2-(N,N-dimethylaminomethyl)-6-methoxyphenyl, 2-(N,N-dimethylaminomethyl)-3-methoxyphenyl, 4-(N,N-dimethylaminomethyl)-3-methoxyphenyl, 3-(morpholinomethyl)phenyl, 2-(morpholinomethyl)phenyl, 4-(morpholinomethyl)phenyl, 3-cyano-4-methoxyphenyl, 2-cyano-4-methoxyphenyl, 3-cyano-5-methoxyphenyl, 2-cyano-5-methoxyphenyl, 2-cyano-6-methoxyphenyl, 2-cyano-3-methoxyphenyl, 4-cyano-3-methoxyphenyl, 4-aminomethyl-3-methylphenyl, 2-aminomethyl-4-methylphenyl, 3-aminomethyl-5-methylphenyl, 3-aminomethyl-4-methylphenyl, 2-aminomethyl-5-methylphenyl, 2-aminomethyl-6-methylphenyl, 2-aminomethyl-3-methylphenyl, (1-methyl)cyclopropyl, (2-methyl)cyclopropyl, 1-fluorocyclopropyl, 4-(2-methyl)pyridinyl, 3-(4-methyl)-pyridinyl, 2-(4-methyl)-pyridinyl, 2-(5-methyl)-pyridinyl, 2-(6-methyl)-pyridinyl, 2-(3-methyl)-pyridinyl, 2-(3-acetamido)-pyridinyl, 2-(4-acetamido)-pyridinyl, 2-(5-acetamido)-pyridinyl, 2-(6-acetamido)-pyridinyl, 3-(2-acetamido)-pyridinyl, 3-(4-acetamido)-pyridinyl, 3-(5-acetamido)-pyridinyl, 3-(6-acetamido)-pyridinyl, 4-(2-acetamido)-pyridinyl, 4-(3-acetamido)-pyridinyl, 4-(N-methylsulfamoyl)phenyl, 3-(N-methylsulfamoyl)phenyl, 2-(N-methylsulfamoyl)phenyl, 4-(N,N-dimethylsulfamoyl)phenyl, 3-(N,N-dimethylsulfamoyl)phenyl, 2-(N,N-dimethylsulfamoyl)phenyl, 3-(N-methylsulfamoyl)pyrrolyl, 3-(N,N-dimethylsulfamoyl)pyrrolyl, 4-(N-methylamido)phenyl, 3-(N-methylamido)phenyl, 2-(N-methylamido)phenyl, 4-(N-methylaminomethyl)phenyl, 3-(N-methylaminomethyl)phenyl, 2-(N-methylaminomethyl)phenyl, 3-(N-methylaminomethyl)-4-methoxyphenyl, 3-(N-methylaminomethyl)-5-methoxyphenyl, 2-(N-methylaminomethyl)-4-methoxyphenyl, 2-(N-methylaminomethyl)-5-methoxyphenyl, 2-(N-methylaminomethyl)-6-methoxyphenyl, 4-(N-methylaminomethyl)-3-methoxyphenyl, 2-(N-rnethylaminomethyl)-3-methoxyphenyl, 4-(acetylamino)phenyl, 3-(acetylamino)phenyl, 2-(acetylamino)phenyl, and ethynyl, 2-(5-N,N-dimethylaminomethyl)thiophenyl, 5-(2-methyl)indazolyl, 5-(3-methyl)indazolyl, 5-(7-methyl)indazolyl, 5-1H-indazolyl, 6-1H-indazolyl, 3-(1-methyl)pyrrolyl, 3-(2-methoxycarbonyl)pyrrolyl, 4-(2-methoxy)pyridinyl, 4-(1H-pyrrolo[2,3-b]pyridinyl), 5-(1H-pyrrolo[2,3-b]pyridinyl), 2-methyl-5-(1H-pyrrolo[2,3-b]pyridinyl), 4-(pyrazol-1-yl)phenyl, 4-(1H-pyrazol-5-yl)phenyl, 4-(1H-pyrazol-4y1)phenyl, 4-(1H-pyrazol-3-yl)phenyl, 4-carboxy-3-nnethylphenyl, 3-1H-pyrazolyl, 4-1H-pyrazolyl, 5-1H pyrazolyl, 4-1H-benzimidazolyl, 5-1H-benzimidazolyl, 1-methyl-5-benzimidazolyl, 2-methyl-5-1H-benzimidazolyl, 1-methyl-6-benzimidazolyl, 2-hydroxy-5-1H-benzimidazolyl, 5-(2-methyl)-benzoxazolyl, 5-(1-methyl)indolyl, 5-(3-methyl)indolyl, 4-1H-indazolyl, 3-(hydroxymethyl)phenyl, 3-hydroxyphenyl, 1,3-benzodioxol-5-yl and 1,2,3-benzotriazol-6-yl, 3-methyl-5-(1H-pyrazolo[3,4-b]pyridinyl, 1-methyl-5-(1H-pyrrazolo[3,4-b]pyridinyl, 2-amino-5-pyrimidinyl, 1,5-naphthyl-3-yl, 1,5-naphthyridin-3-yl, 5-benzofuranyl, 6-(2-methyl)-benzothiazolyl, 5-(2-methyl)-benzothiazolyl, 5-benzoxazolyl, 6-benzoxazolyl, 6-(2-methyl)-benzoxazolyl, 5-(2-methyl)-benzoxazolyl, 4-((2-methoxyethoxy)methyl)phenyl, 4-(cyclopropylmethoxy)methyl)phenyl, 3-(2-(aminomethyl)-1,5-dimethyl)-pyrrolyl, 5-oxoisoindolinyl, 3-fluoro-4-pyrrolidin-1-yl-phenyl, 4-(1-aminocarbonylmethyl)-pyrazolyl, 4-(1-oxetan-3-yl)-pyrazolyl, 4-(1-amino-2-methyl-2-propyl)phenyl, 4-1-(pyrrolidin-1-yl)ethyl)phenyl, 4-(1-dimethylamino)ethyl)phenyl, 4-(2-hydroxypropan-2-yl)phenyl, 4-(2-methyl, 1-methylamino-propan-2-yl)phenyl, 4-(2-methyl, 1-dimethylamino-propan-2-yl)phenyl, 4-(1-amino-2-hydroxypropan-2-yl)phenyl and 3-dimethylaminoethyl-4-methoxyphenyl.

13. The compound according to claim 1, having the following Formula (III): 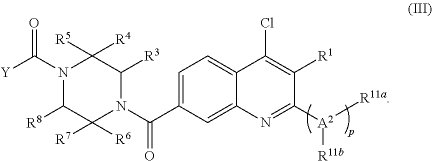
wherein R¹ and R^{11a} are independently selected from the group consisting of H, halogen, cyano, optionally substituted alkyl, optionally substituted alkene, optionally substituted alkyne, optionally substituted alkoxy, optionally substituted amino, optionally substituted cycloalkyl, optionally substituted heterocycloalkyl, optionally substituted aryl and optionally substituted heteroaryl;
R^{11b} is absent, H or optionally substituted alkyl;
A² is selected from CH, N, O or S; and
p is an integer selected from from 0, 1 or 2.

14. The compound according to claim 13, having the following Formula (IIIa):

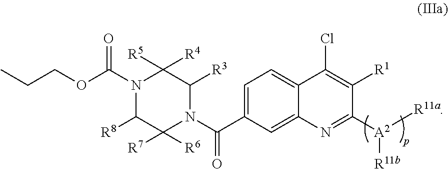

15. The compound according to claim 13, wherein R¹ and R^{11a} are independently selected from the group consisting of a bond, H, cyano, methyl, ethyl, ethynyl, phenyl, 2-methylphenyl, 3-methylphenyl, 4-methylphenyl, 2-hydroxyphenyl, 3-hydroxyphenyl, 4-hydroxyphenyl, 2-(trifluoromethyl)phenyl, 3-(trifluoromethyl)phenyl, 4-(trifluoromethyl)phenyl, 2-chlorophenyl, 3-chlorophenyl, 4-chlorophenyl, 2-fluorophenyl, 3-fluorophenyl, 4-fluorophenyl, 2-fluoromethylphenyl, 3-fluoromethylphenyl, 4-fluoromethylphenyl, 2-hydroxymethylphenyl, 3-hydroxymethylphenyl, 4-hydroxymethylphenyl, 2-methoxyphenyl, 3-methoxyphenyl, 4-methoxyphenyl, 2-ethoxyethylphenyl, 3-ethoxyethylphenyl, 4-ethoxyethylphenyl, 2-(azidomethyl)phenyl, 3-(azidomethyl)phenyl, 4-(azidomethyl)phenyl, 2,3-difluorophenyl, 2,4-difluorophenyl, 2,5-difluorophenyl, 2,6-difluorophenyl, 3,4-difluorophenyl, 3,5-difluorophenyl, 3,5-difluoro-4-hydroxyphenyl, 3,5-difluoro-4-(aminocarbonyl)phenyl, 3,5-difluoro-4-aminomethylphenyl, 2-cyanophenyl, 3-cyanophenyl, 4-cyanophenyl, 2-(cyanomethyl)phenyl, 3-(cyanomethyl)phenyl, 4-(cyanomethyl)phenyl, 2-nitrophenyl, 3-nitrophenyl, 4-nitrophenyl, 2-aminophenyl, 3-aminophenyl, 4-aminophenyl, 2-(aminomethyl)phenyl, 3-(aminomethyl)phenyl, 4-(aminomethyl)phenyl, 2-(dimethylamino)phenyl, 3-(dimethylamino)phenyl, 4-(dimethylamino)phenyl, 2-(aminocarbonyl)phenyl, 3-(aminocarbonyl)phenyl, 4-(aminocarbonyl)phenyl, 2-(methylaminocarbonyl)phenyl, 3-(methylaminocarbonyl)phenyl, 4-(methylaminocarbonyl)phenyl, 2-(ethylaminocarbonyl)phenyl, 3-(ethylaminocarbonyl)phenyl, 4-(ethylaminocarbonyl)phenyl, 4-(1-ethoxyethyl)phenyl, 4-(2-hydroxy-2-propyl)phenyl, 2-pyridinyl, 3-pyridinyl, 4-pyridinyl, 2-methyl-3-pyridinyl, 4-methyl-3-pyridinyl, 5-methyl-3-pyridinyl, 6-methyl-3-pyridinyl, 6-methoxycarbonyl-3-pyridinyl, thiophenyls such as 2-thiophenyl, 3-thiophenyl, 1-pyrrolidinyl, 2-pyrrolidinyl, 3-pyrrolidinyl, 1-pyrrolyl, 2-pyrrolyl, 3-pyrrolyl, 1-methyl-3-pyrrolyl, 3-(1,2,5-trimethyl)-pyrrolyl, 2-ethynylphenyl, 3-ethynylphenyl, 4-ethynylphenyl, 2-ethylphenyl, 3-ethylphenyl, 4-ethylphenyl, 2-(1-hydroxyethyl)phenyl, 3-(1-hydroxyethyl) phenyl, 4-(1-hydroxyethyl)phenyl, 2-(2-hydroxyethyl)phenyl, 3-(2-hydroxyethyl)phenyl, 4-(2-hydroxyethyl)phenyl, 4-fluoro-3-methylphenyl, 4-fluoro-2-methylphenyl, 3-fluoro-2-methylphenyl, 3-fluoro-4-methylphenyl, 3-fluoro-5-methylphenyl, 2-fluoro-5-methylphenyl, 4-fluoro-3-methoxyphenyl, 4-fluoro-2-methoxyphenyl, 3-fluoro-2-methoxyphenyl, 3-fluoro-4-methoxyphenyl, 3-fluoro-5-methoxyphenyl, 2-fluoro-5-methoxyphenyl, 4-fluoro-3-hydroxyphenyl, 4-fluoro-2-hydroxyphenyl, 4-hydroxy-3-fluorophenyl, 4-hydroxy-2-fluorophenyl, 4-fluoro-3-hydroxymethylphenyl, 4-fluoro-2-hydroxymethylphenyl, 3-fluoro-2-hydroxymethylphenyl, 3-fluoro-4-hydroxymethylphenyl, 3-fluoro-5-hydroxymethylphenyl, 2-fluoro-5-hydroxymethylphenyl, 3-fluoro-4-(2-hydroxy-2-propyl)phenyl, 3-(aminocarbonyl)-4-fluorophenyl, 2-(aminocarbonyl)-4-fluorophenyl, 2-(aminocarbonyl)-3-fluorophenyl, 4-(aminocarbonyl)-3-fluorophenyl, 5-(aminocarbonyl)-3-fluorophenyl, 5-(aminocarbonyl)-2-fluorophenyl, 4-fluoro-3-(methylaminocarbonyl)phenyl, 3-fluoro-4-(methylaminocarbonyl)phenyl, 4-fluoro-2-(methylaminocarbonyl)phenyl, 3-fluoro-2-(methylaminocarbonyl)phenyl, 4-(cyclopropylaminocarbonyl)phenyl, 2-(cyclopropylaminocarbonyl)phenyl, 3-(cyclopropylaminocarbonyl)phenyl, 4-fluoro-3-(cyclopropylaminocarbonyl)phenyl, 3-fluoro-4-(cyclopropylaminocarbonyl)phenyl, 4-fluoro-2-(cyclopropylaminocarbonyl)phenyl, 3-fluoro-2-(cyclopropylaminocarbonyl)phenyl, (3-fluoro-4-(dimethylaminocarbonyl)phenyl, 3-fluoro-5-(dimethylaminocarbonyl)phenyl, 2-fluoro-5-(dimethylaminocarbonyl)phenyl, 4-fluoro-3-(dimethylaminocarbonyl)phenyl, 4-fluoro-2-(dimethylaminocarbonyl)phenyl, 3-fluoro-2-(dimethylaminocarbonyl)phenyl, 3-methyl-4-(methylaminocarbonyl)phenyl, 3-amino-4-fluorophenyl, 2-amino-4-fluorophenyl, 3-aminomethyl-4-fluorophenyl, 2-aminomethyl-4-fluorophenyl, 3-hydroxymethyl-4-methylphenyl, 2-hydroxymethyl-4-methyl-phenyl, 2-hydroxymethyl-3-methyl-phenyl, 4-hydroxymethyl-3-methylphenyl, 5-hydroxymethyl-3-methylphenyl, 5-hydroxymethyl-2-methylphenyl, 2-morpholinophenyl, 3-morpholinophenyl, 4-morpholinophenyl, 2-(pyrrolidin-1-yl)phenyl, 3-(pyrrolidin-1-yl)phenyl, 4-(pyrrolidin-1-yl)phenyl, 4-(1-amino-1-cyclopropyl)phenyl, cyclopropyl, cyclobutyl, cyclopentyl, cyclohexyl, 2-thiazolyl, 4-thiazolyl, 5-thiazolyl, 2-methylthiazolyl, 4-methylthiazolyl, 4-(dimethylamido)phenyl, 2-(dimethylamido)phenyl, 3-(dimethylamido)phenyl, 2-benzylamin, 3-benzylamin, 4-benzylamin, 2-methylaminophenyl, 3-methylaminophenyl, 4-methylaminophenyl, 6-(1-methyl)indazolyl, 6-(2-methyl)indazolyl, 5-(1-methyl)indazolyl, 5-(2-methyl)indazolyl, 4-(1-methyl)indazolyl, 3-(1-methyl)indazolyl, 5-(3-methyl)indazolyl, 5-(1-methyl)pyrazolyl, 4-(1-methyl)-pyrazolyl, 3-(1-methyl) pyrazolyl, 4-(1-isopropyl)-pyrazolyl, 4-(1-difluoromethyl)-pyrazolyl, 4-(5-trifluoromethyl)-pyrazolyl, 4-(1-(2,2,2)-trifluoroethyl)pyrazolyl, 4-(1-cyclopentyl)pyrazolyl, 2-(1-methyl) pyrazolyl-phenyl, 3-(1-methyl) pyrazolyl-phenyl, 4-(imidazol-1-yl)phenyl, 1-imidazolyl, 2-imidazolyl, 3-imidazolyl, 4-(N,N-dimethylsulfamoyl)phenyl, 3-(N,N-dimethylsulfamoyl)phenyl, 2-(N,N-dimethylsulfonamoyl)phenyl, 4-(4-methylpiperazino)phenyl, 3-(4-methylpiperazino)phenyl, 2-(4-methylpiperazino)phenyl, 3-[1,2,4]-triazol-4-ylphenyl, 2-[1,2,4]-triazol-4-yl phenyl, 4-[1,2,4]-triazol-4-ylphenyl, 3-(aminomethyl)-4-methoxyphenyl, 3-(aminomethyl)-5-methoxyphenyl, 2-(aminomethyl)-4-methoxyphenyl, 2-(aminomethyl)-5-methoxyphenyl, 2-(aminomethyl)-6-methoxyphenyl, 4-(aminomethyl)-3-methoxyphenyl, 2-(aminomethyl)-3-methoxyphenyl, 4-(dimethylaminomethyl)phenyl, 3-(dimethylaminomethyl)phenyl, 2-(dimethylaminomethyl)phenyl, 4-fluoro-3-(dimethylaminomethyl)phenyl, 4-fluoro-2-(dimethylaminomethyl)phenyl, 4-methoxy-3-methylphenyl, 2-methoxy-4-methylphenyl, 3-methoxy-5-methylphenyl, 3-methoxy-4-methylphenyl, 2-methoxy-5-methylphenyl, 2-methoxy-6-methylphenyl, 2-methoxy-3-methylphenyl, 4-methoxy-3-hydroxymethylphenyl, 3-methoxy-4-hydroxymethylphenyl, 2-methoxy-4-hydroxymethylphenyl, 3-methoxy-5-hydroxymethylphenyl, 2-methoxy-5-hydroxymethylphenyl, 2-methoxy-6-hydroxymethylphenyl, 2-methoxy-3-hydroxymethylphenyl, 4-hydroxy-3-hydroxymethylphenyl, 4-hydroxy-3-methylphenyl, 3-ethoxy-4-hydroxyphenyl, 3-hydroxy-4-methylphenyl, 2-hydroxy-4-methylphenyl, 3-cyano-4-methylphenyl, 4-cyano-3-methylphenyl, 2-cyano-4-methylphenyl, 3-cyano-5-methylphenyl, 2-cyano-5-methylphenyl, 2-cyano-6-methylphenyl, 2-cyano-3-methylphenyl, 4-(aminosulfonyl)phenyl, 3-(aminosulfonyl)phenyl, 2-(aminosulfonyl)phenyl, 3-(N,N-dimethylaminomethyl)-4-methoxyphenyl, 3-(N,N-dimethylaminomethyl)-5-methoxyphenyl, 2-(N,N-dimethylaminomethyl)-4-methoxyphenyl, 2-(N,N-dimethylaminomethyl)-5-methoxyphenyl, 2-(N,N-dimethylaminomethyl)-6-methoxyphenyl, 2-(N,N-dimethylaminomethyl)-3-methoxyphenyl, 4-(N,N-dimethylaminomethyl)-3-methoxyphenyl, 3-(morpholinomethyl)phenyl, 2-(morpholinomethyl)phenyl, 4-(morpholinomethyl)phenyl, 3-cyano-4-methoxyphenyl, 2-cyano-4-methoxyphenyl, 3-cyano-5-methoxyphenyl, 2-cyano-5-methoxyphenyl, 2-cyano-6-methoxyphenyl, 2-cyano-3-methoxyphenyl, 4-cyano-3-methoxyphenyl, 4-aminomethyl-3-methylphenyl, 2-aminomethyl-4-methylphenyl, 3-aminomethyl-5-methylphenyl, 3-aminomethyl-4-methylphenyl, 2-aminomethyl-5-methylphenyl, 2-aminomethyl-6-methylphenyl, 2-aminomethyl-3-methylphenyl, (1-methyl)cyclopropyl, (2-methyl)cyclopropyl, 1-fluorocyclopropyl, 4-(2-methyl)pyridinyl, 3-(4-methyl)-pyridinyl, 2-(4-methyl)-pyridinyl, 2-(5-methyl)-pyridinyl, 2-(6-methyl)-pyridinyl, 2-(3-methyl)-pyridinyl, 2-(3-acetamido)-pyridinyl, 2-(4-acetamido)-pyridinyl, 2-(5-acetamido)-pyridinyl, 2-(6-acetamido)-pyridinyl, 3-(2-acetamido)-pyridinyl, 3-(4-acetamido)-pyridinyl, 3-(5-acetamido)-pyridinyl, 3-(6-acetamido)-pyridinyl, 4-(2-acetamido)-pyridinyl, 4-(3-acetamido)-pyridinyl, 4-(N-methylsulfamoyl)phenyl, 3-(N-methylsulfamoyl)phenyl, 2-(N-methylsulfamoyl)phenyl, 4-(N,N-dimethylsulfamoyl)phenyl, 3-(N,N-dimethylsulfamoyl)phenyl, 2-(N,N-dimethylsulfamoyl)phenyl, 3-(N-methylsulfamoyl)pyrrolyl, 3-(N,N-dimethylsulfamoyl)pyrrolyl, 4-(N-methylamido)phenyl, 3-(N-methylamido)phenyl, 2-(N-methylamido)phenyl, 4-(N-methylaminomethyl)phenyl, 3-(N-methylaminomethyl)phenyl, 2-(N-methylaminomethyl)phenyl, 3-(N-methylaminomethyl)-4-methoxyphenyl, 3-(N-methylaminomethyl)-5-methoxyphenyl, 2-(N-methylaminomethyl)-4-methoxyphenyl, 2-(N-methylaminomethyl)-5-methoxyphenyl, 2-(N-methylaminomethyl)-6-methoxyphenyl, 4-(N-methylaminomethyl)-3-methoxyphenyl, 2-(N-methylaminomethyl)-3-methoxyphenyl, 4-(acetylamino)phenyl, 3-(acetylamino)phenyl, 2-(acetylamino)phenyl, and ethynyl, 2-(5-N,N-dimethylaminomethyl)thiophenyl, 5-(2-methyl)indazolyl, 5-(3-methyl)indazolyl, 5-(7-methyl)indazolyl, 5-1H-indazolyl, 6-1H-indazolyl, 3-(1-methyl)pyrrolyl, 3-(2-methoxycarbonyl)pyrrolyl, 4-(2-methoxy)pyridinyl, 4-(1H-pyrrolo[2,3-b]pyridinyl), 5-(1H-pyrrolo[2,3-b]pyridinyl), 2-methyl-5-(1H-pyrrolo[2,3-b]pyridinyl), 4-(pyrazol-1-yl)phenyl, 4-(1H-pyrazol-5-yl)phenyl, 4-(1H-pyrazol-4yl)phenyl, 4-(1H-pyrazol-3-yl)phenyl, 4-carboxy-3-methylphenyl, 3-1H-pyrazolyl, 4-1H-pyrazolyl, 5-1H pyrazolyl, 4-1H-benzimidazolyl, 5-1H-benzimidazolyl, 1-methyl-5-benzimidazolyl, 2-methyl-5-1H-benzimidazolyl, 1-methyl-6-benzimidazolyl, 2-hydroxy-5-1H-benzimidazolyl, 5-(2-methyl)-benzoxazolyl, 5-(1-methyl)indolyl, 5-(3-methyl)indolyl, 4-1H-indazolyl, 3-(hydroxymethyl)phenyl, 3-hydroxyphenyl, 1,3-benzodioxol-5-yl and 1,2,3-benzotriazol-6-yl, 3-methyl-5-(1H-pyrazolo[3,4-b]pyridinyl, 1-methyl-5-(1H-pyrrazolo[3,4-b]pyridinyl, 2-amino-5-pyrimidinyl, 1,5-naphthyl-3-yl, 1,5-naphthyridin-3-yl, 5-benzofuran, 6-(2-methyl)-benzothiazolyl, 5-(2-methyl)-benzothiazolyl, 5-benzoxazolyl, 6-benzoxazolyl, 6-(2-methyl)-benzoxazol, 4-((2-methoxyethoxy)methyl)phenyl, 4-(cyclopropylmethoxy)methyl)phenyl, 3-(2-(aminomethyl)-1,5-dimethyl)-pyrrolyl, 5-oxoisoindolinyl, 3-fluoro-4-pyrrolidin-1-yl-phenyl, 4-(1-aminocarbonylmethyl)-pyrazolyl, 4-(1-oxetan-3-yl)-pyrazolyl, 4-(1-amino-2-methyl-2-propyl)phenyl, 4-1-(pyrrolidin-1-yl)ethyl)phenyl, 4-(1-dimethylamino)ethyl)phenyl, 4-(2-hydroxypropan-2-yl)phenyl, 4-(2-methyl, 1-methylamino-propan-2-yl)phenyl, 4-(2-methyl, 1-dimethylamino-propan-2-yl)phenyl, 4-(1-amino-2-hydroxypropan-2-yl)phenyl and 3-dimethylaminoethyl-4-methoxyphenyl, or wherein R¹ and R² are taken together to form an optionally substituted 5-membered cycloalkyl, an optionally substituted 6-membered cycloalkyl, an optionally substituted 5-membered heterocycloalkyl or an optionally substituted 6-membered heterocycloalkyl.

16. (canceled)

17. The compound according to claim 15, having the following Formula (IV): 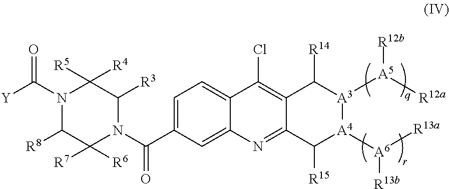
wherein
A³ and A⁴ are independently selected from CH or N;
A⁵ and A⁶ are independently selected from CH, N, O or S;
R^{12a}, R^{13a}, R¹⁴ and R¹⁵ are independently selected from the group consisting of H, halogen, optionally substituted alkyl, optionally substituted alkene, optionally substituted alkyne, optionally substituted alkoxy, optionally substituted amino, optionally substituted acyl, optionally substituted cycloalkyl, optionally substituted heterocycloalkyl, optionally substituted aryl and optionally substituted heteroaryl; or wherein R12a, R13a, R14 and R15 are independently selected from H, methyl ,ethyl, propyl, butyl, halogen, cyano, COOMe, COOEt, phenyl, 2-pyridinyl, 3-pyridinyl, 4-pyridinyl, 2-(3-methyl)pyridinyl, 2-(4-methyl)pyridinyl, 2-(5-methyl)pyridinyl, 2-(6-methyl)pyridinyl, 3-(2-methyl)pyridinyl, 3-(4-methyl)pyridinyl, 3-(5-methyl)pyridinyl, 3-(6-methyl)pyridinyl, 4-(2-methyl)pyridinyl, 4-(3-fluoro)pyridinyl, 2-(3-fluoro)pyridinyl, 2-(4-fluoro)pyridinyl, 2-(5-fluoro)pyridinyl, 2-pyrazinyl, 2-(6-fluoro)pyridinyl, 3-(2-fluoro)pyridinyl, 3-(4-fluoro)pyridinyl, 3-(5-fluoro)pyridinyl, 3-(6-fluoro)pyridinyl, 4-(2-fluoro)pyridinyl, 4-(3-fluoro)pyridinyl, 2-(3-cyano)pyridinyl, 2-(4-cyano)pyridinyl, 2-(5-cyano)pyridinyl, 2-(6-cyano)pyridinyl, 3-(2-cyano)pyridinyl, 3-(4-cyano)pyridinyl, 3-(5-cyano)pyridinyl, 3-(6-cyano)pyridinyl, 4-(2-cyano)pyridinyl, 2-[3-(aminocarbonyl)]pyridinyl, 2-[4-(aminocarbonyl)]pyridinyl, 2-[5-(aminocarbonyl)]pyridinyl, 2-[6-(aminocarbonyl)]pyridinyl, 3-[2-(aminocarbonyl)]pyridinyl, 3-[4-(aminocarbonyl)]pyridinyl, 3-[5-(aminocarbonyl)]pyridinyl, 3-[6-(aminocarbonyl)]pyridinyl, 4-[2-(aminocarbonyl)]pyridinyl, 2-[3-(aminomethyl)]pyridinyl, 2-[4-(aminomethyl)]pyridinyl, 2-[5-(aminomethyl)]pyridinyl, 2-[6-(aminomethyl)]pyridinyl, 3-[2-(aminomethyl)]pyridinyl, 3-[4-(aminomethyl)]pyridinyl, 3-[5-(aminomethyl)]pyridinyl, 3-[6-(aminomethyl)]pyridinyl, 4-[2-(aminomethyl)]pyridinyl, 2-pyrimidinyl, 5-pyrimidinyl, 4-pyrimidinyl, 4-(3-methyl)pyrimidyl, 2-thiazolyl, 3-thiazolyl, pyrrolidine, 5-methyl-1,2,4-oxadiazol-3-yl, NH2, N(CH3)2, CH2CH═CH2, CH═CH2, CH2N(CH3)2, CH2NH2, CH2CH2NH2, C(O)NH2, NHC(NH)NH2, CH2NHC(NH)NH2, 2-(4-ethynyl)pyridinyl, 3-(4-ethynyl)pyridinyl, 2-(6-ethynyl)pyridinyl, 2-(5-ethynyl)pyridinyl, 3-(4-ethynyl)pyridinyl, 3-(2-ethynyl)pyridinyl, 3-(5-ethynyl)pyridinyl, 3-(6-ethynyl) pyridinyl, 2-(3-cyano)pyrimidinyl, 2-(5-cyano)pyrimidinyl, 2-(6-cyano)pyrimidinyl, 6-(2-cyano)pyrimidinyl, 2-(1-methyl)pyrazolyl, 4-(1-methyl)pyrazolyl, CH2-pyrrolidine, CH2CH2-pyrrolidine, ethynyl;
R^{12b} and R^{13b} are independently absent, H or an optionally substituted alkyl; and
q and r are independently integers selected from 0, 1 or 2.

18. (canceled)

19. The compound according to claim 17, having the following Formula (IVa): 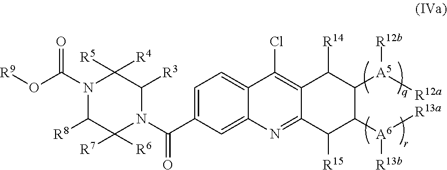
wherein R⁹ is an optionally substituted C₃ to C₁₀ alkyl, optionally substituted C₃ to C₁₀ alkenyl, optionally substituted C₃ to C₁₀ alkynyl or optionally substituted C₃ to C₇ cycloalkyl.

20. (canceled)

21. The compound according to claim 1, wherein R³, R⁴, R⁵, R⁶, R⁷ and R⁸ is independently selected from the group consisting of a bond, H, methyl, (S)-methyl, (R)-methyl, ethyl, (S)-ethyl, (R)-ethyl, cyano, —CH₂OH, (S)—CH₂OH, (R)-CH₂OH, COOCH₃, CH₂OC(O)CH₃, (R)-CH₂OC(O)CH₃, (S)—CH₂OC(O)CH₃, CH₂OC(O)CH₂CH₂OCH₃, (R)—CH₂OC(O)CH₂CH₂OCH₃, (S)—CH₂OC(O)CH₂CH₂OCH₃, CH₂CH₂OH, (R)—CH₂CH₂OH, (S)—CH₂CH₂OH, CH₂OC(O)CH₂CH₂CH₃, (R)—CH₂OC(O)CH₂CH₂CH₃, (S)—CH₂OC(O)CH₂CH₂CH₃, CF₃, (R)—CF₃, (S)—CF₃, (S)—COOCH₃, (R)—COOCH₃, CH₂OCH₃, (S)—CH₂OCH₃, (R)—CH₂OCH₃, CONHCH₃, (S)—CONHCH₃, (R)—CONHCH₃ CH₂COOCH₃, (S)—CH₂COOCH₃, (R)—CH₂COOCH₃, CH₂OC(O)CH(CH₃)₂, (S)—CH₂OC(O)CH(CH₃)₂,CH₂CONHCH₃, (S)—CH₂CONHCH₃, (R)—CH₂CONHCH₃, (R)—CH₂OC(O)CH(CH₃)₂, CONH₂, (S)—CONH₂, (R)—CONH₂, CH₂CON(CH₃)₂, (S)—CH₂CON(CH₃)₂, (R)—CH₂CON(CH₃)₂ and CH₂C(O)NH(CH₃); or
wherein R⁴ and R⁵ or R⁶ and R⁷ may be taken together to form an optionally substituted alkylene bridge or an optionally substituted alkylene bridge wherein one or two alkylene units may be replaced with O, NH or S; or
wherein R⁴ and R⁵ or R⁶ and R⁷ may be taken together to form a cyclopropane; or wherein R³ and R⁴, R³ and R⁵, R³ and R⁶, R³ and R⁷, R³ and R⁸, R⁴ and R⁶, R⁴ and R⁷, R⁴ and R⁸, R⁵ and R⁶, R⁵ and R⁷, R⁵ and R⁸, R⁶ and R⁸ or R⁷ and R⁸ are taken together to form an optionally substituted alkylene bridge or an optionally substituted alkylene bridge wherein one or two alkylene units may be replaced with O, NH or S
wherein the bridge of the optionally substituted alkylene bridge is a 1-carbon, 2-carbon or 3-carbon alkylene bridging group wherein optionally one or two alkylene units are replaced with O, NH or S.

22.-25. (canceled)

26. The compound according to claim 1, selected from the group consisting of: 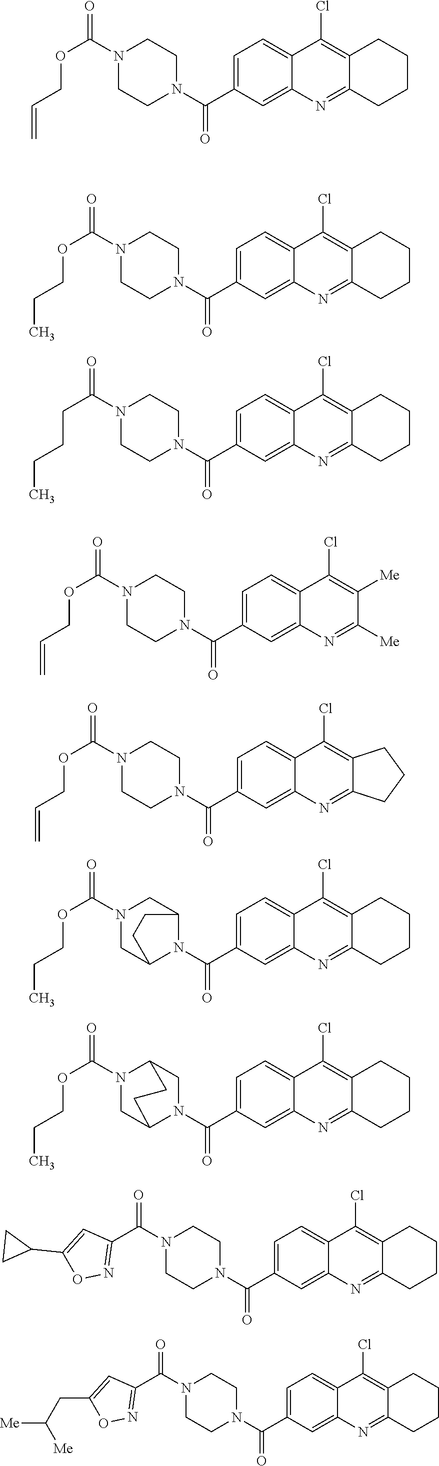 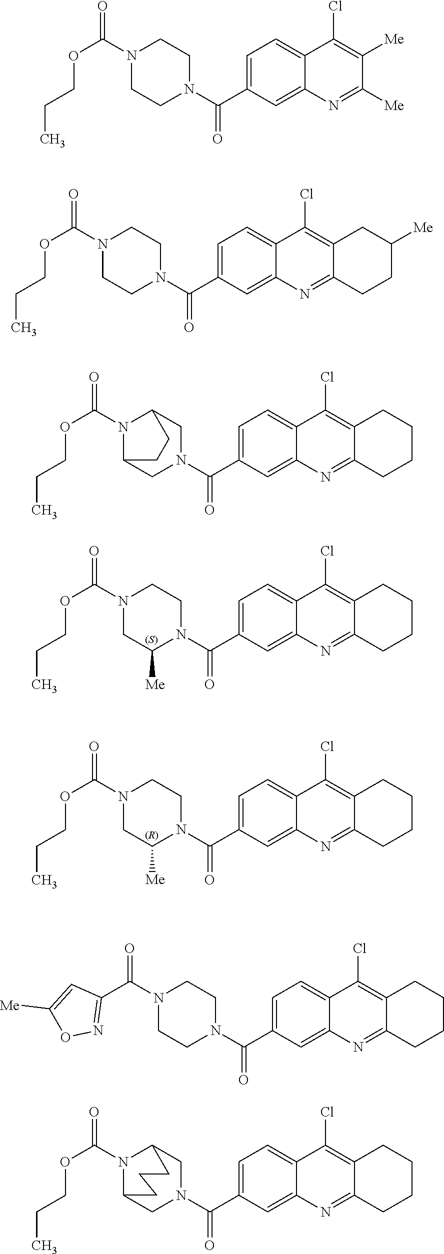 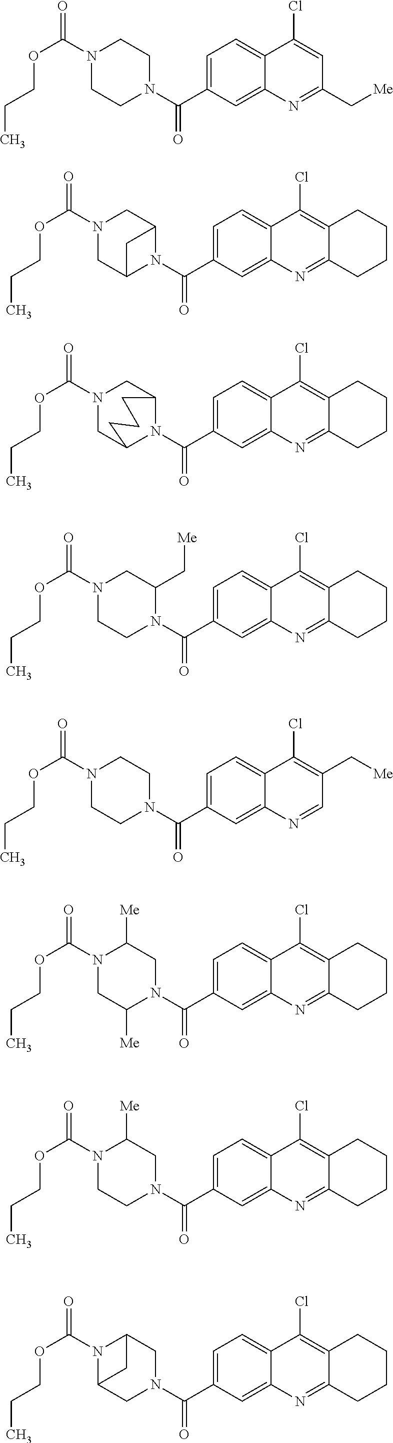 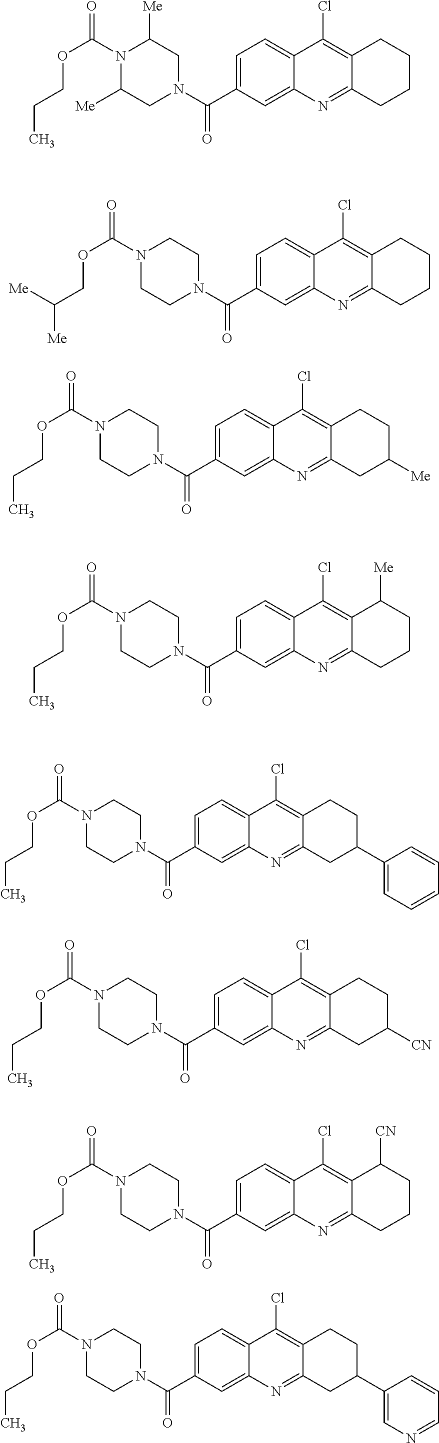 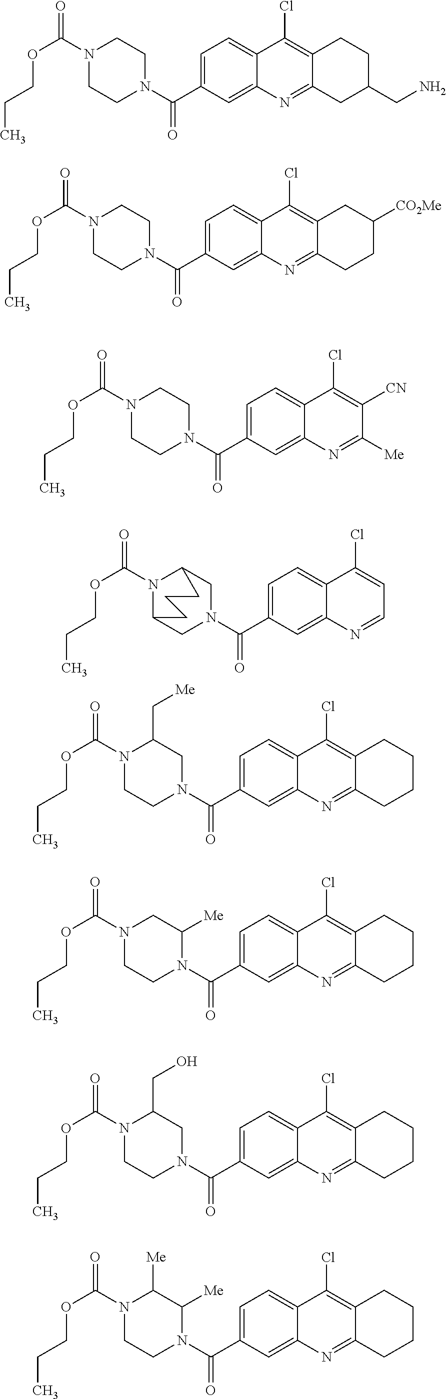 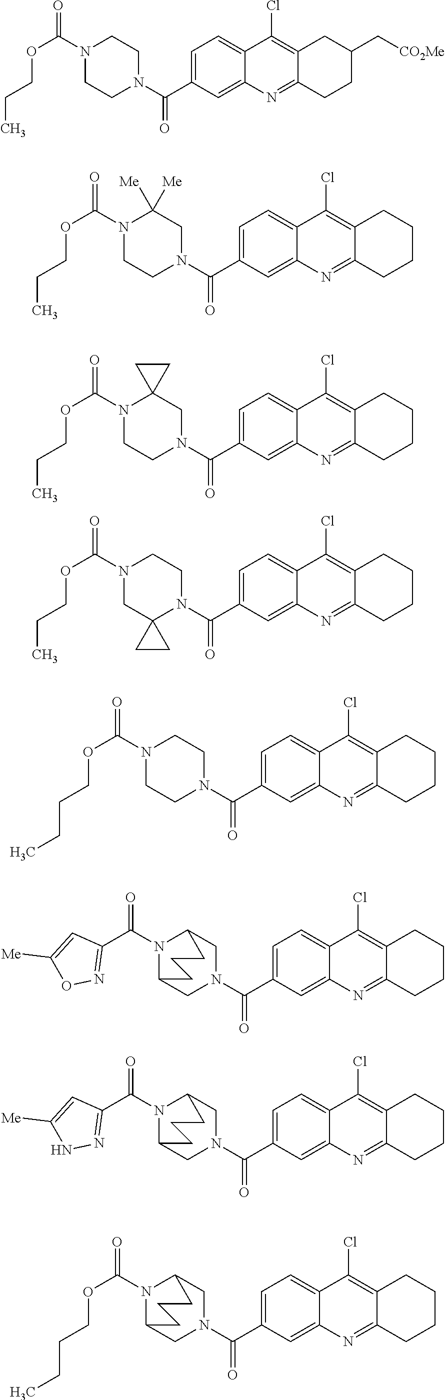 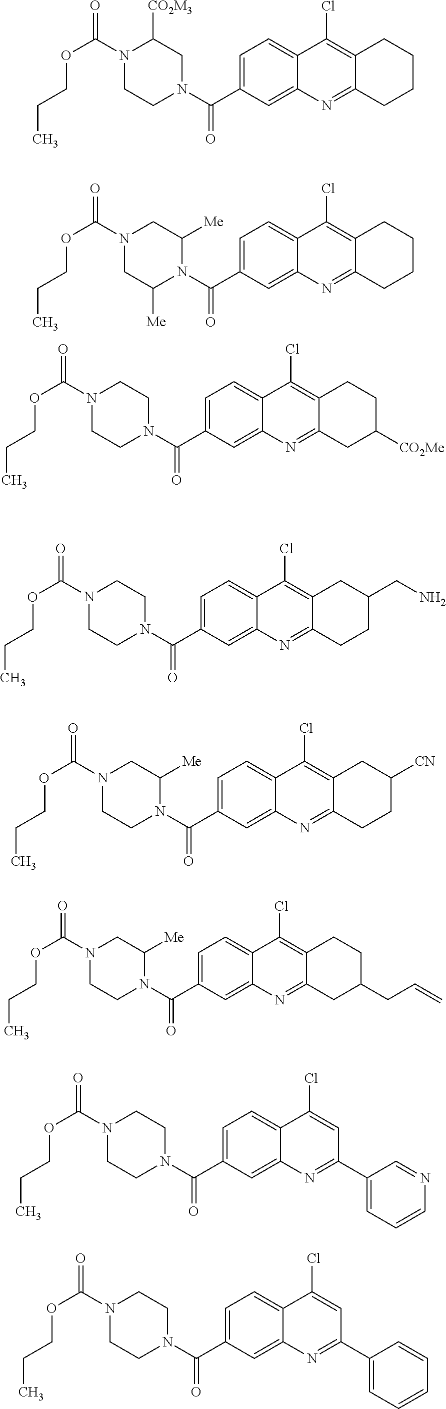 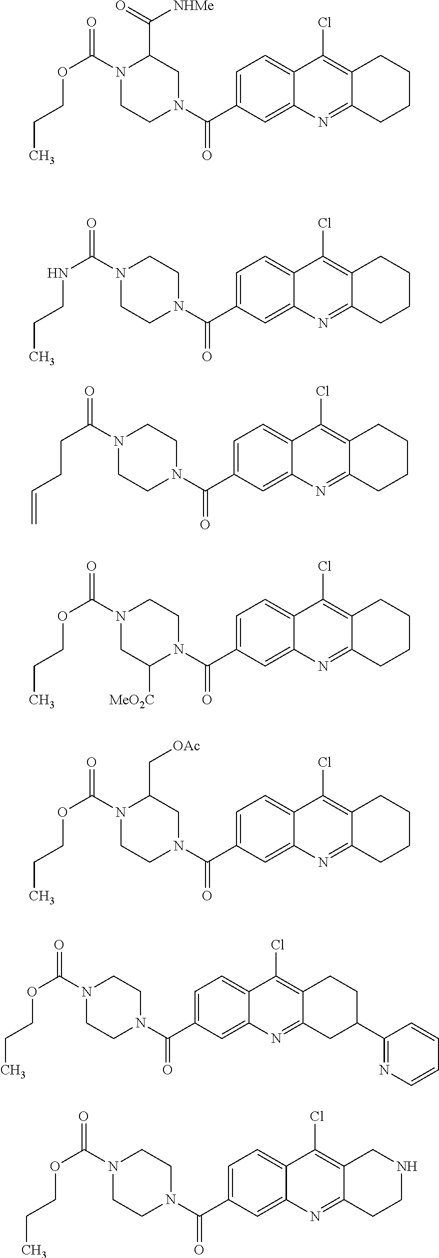 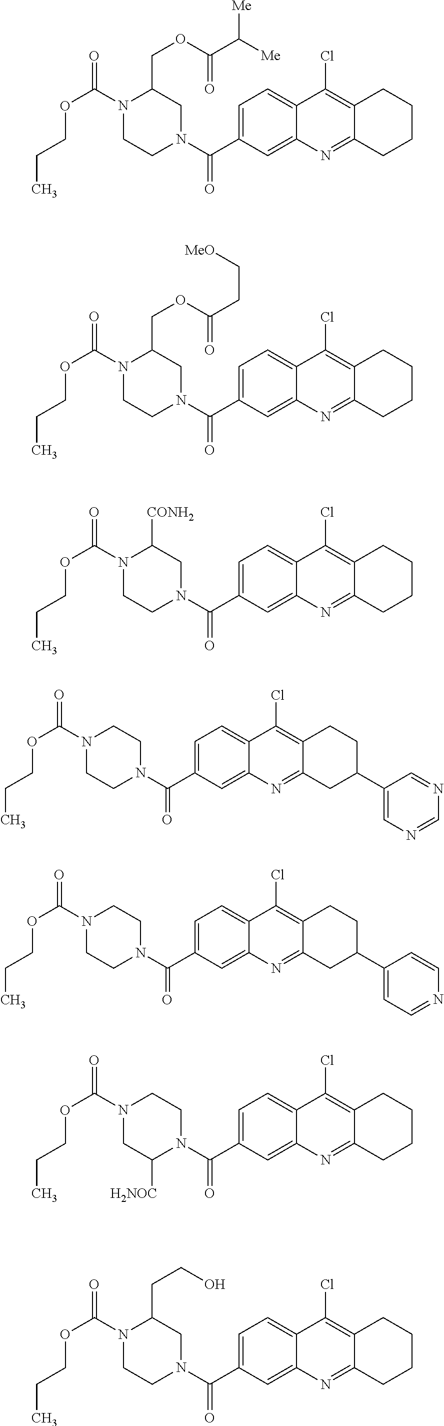 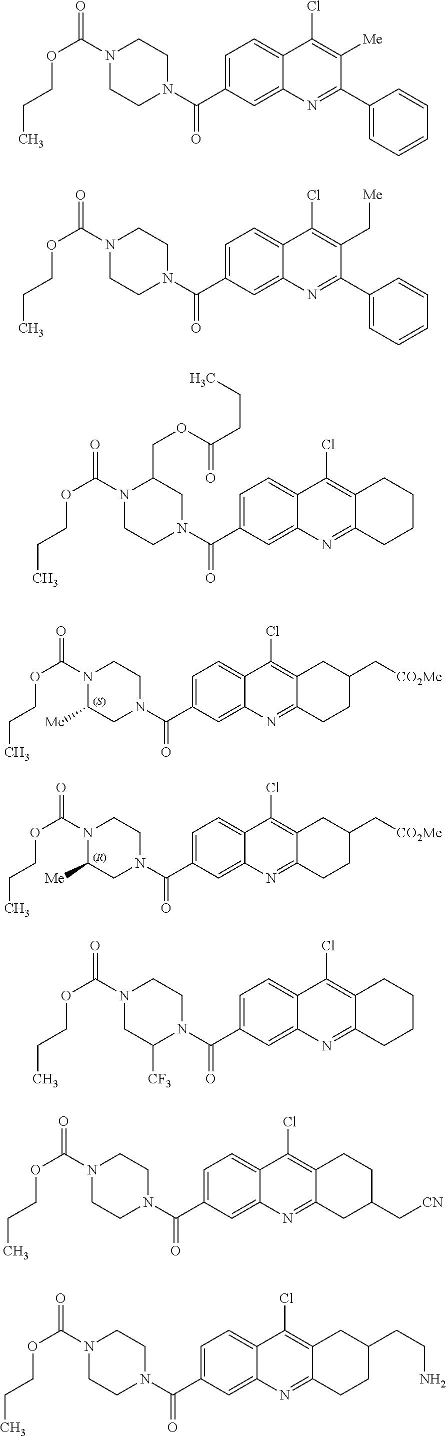 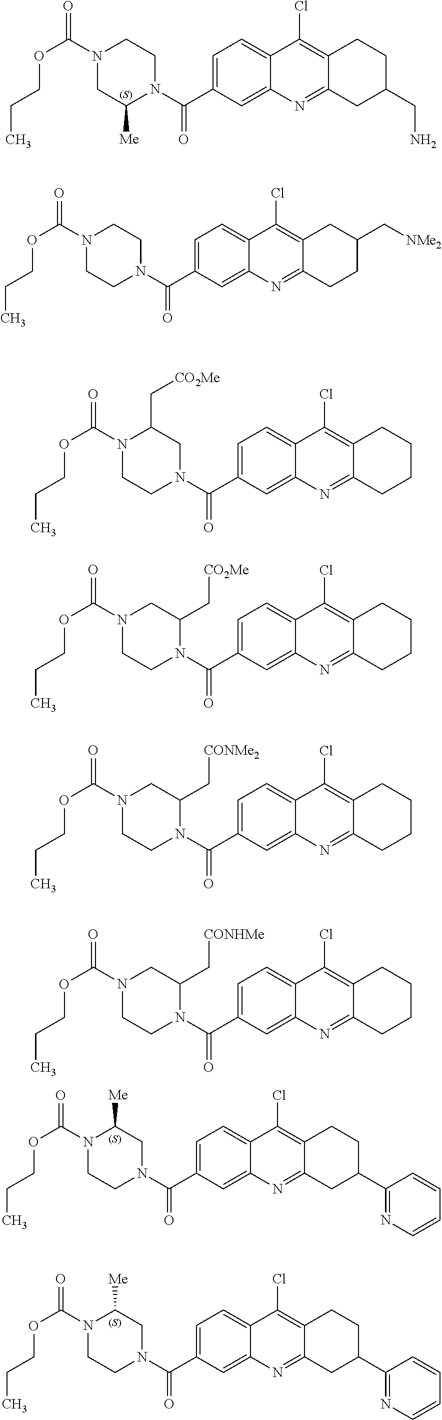 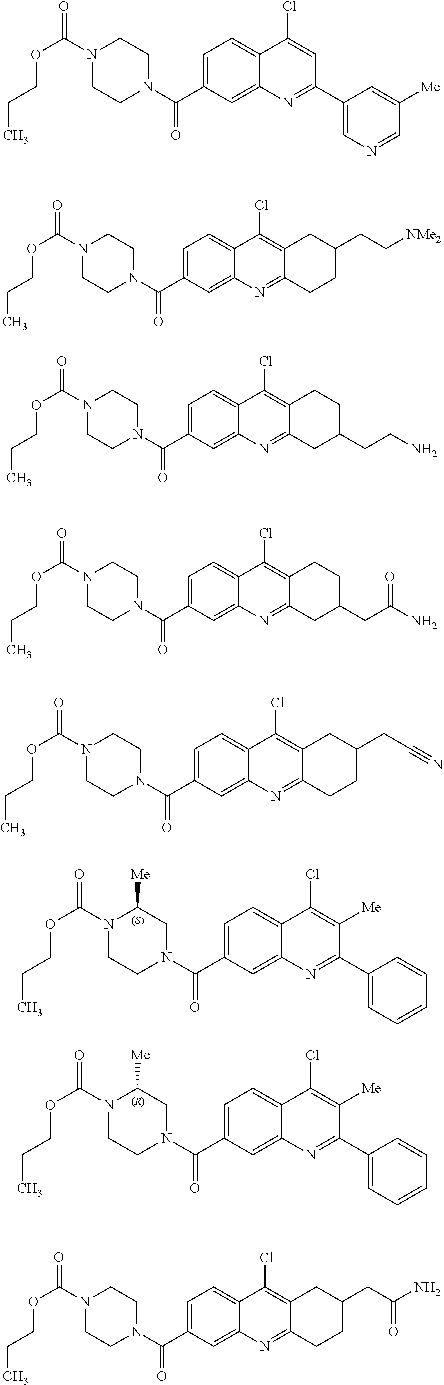 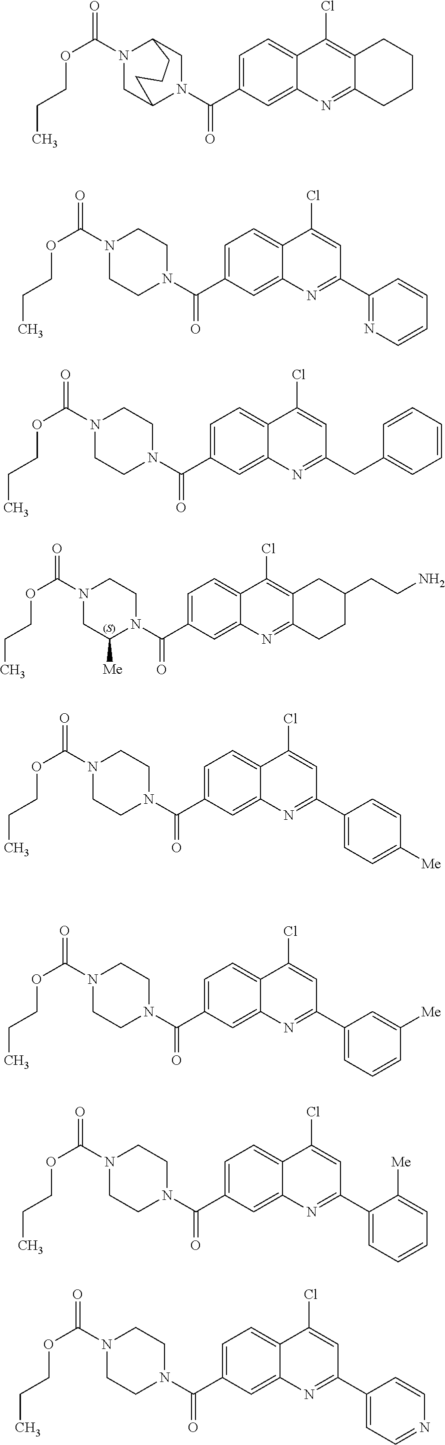 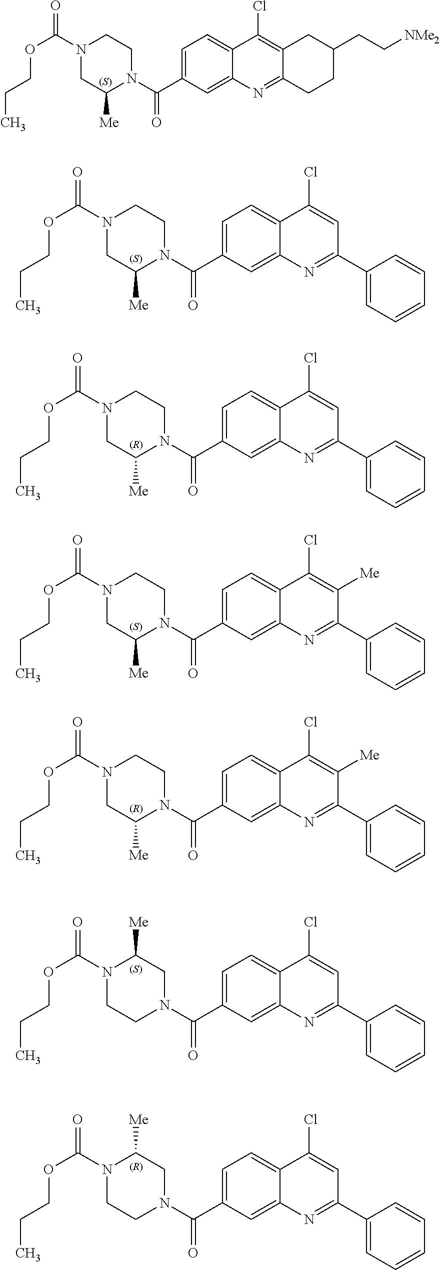 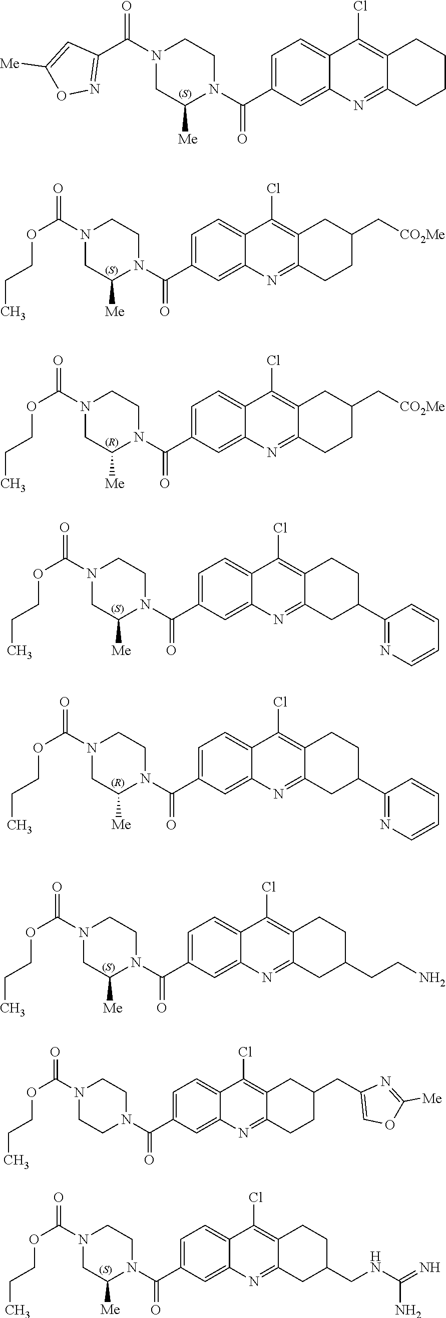 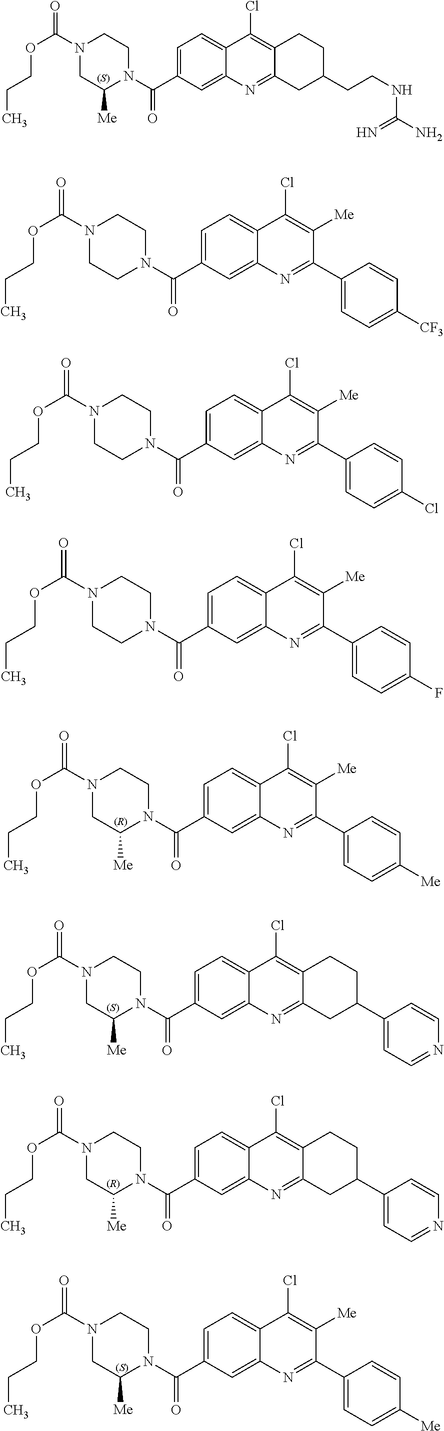 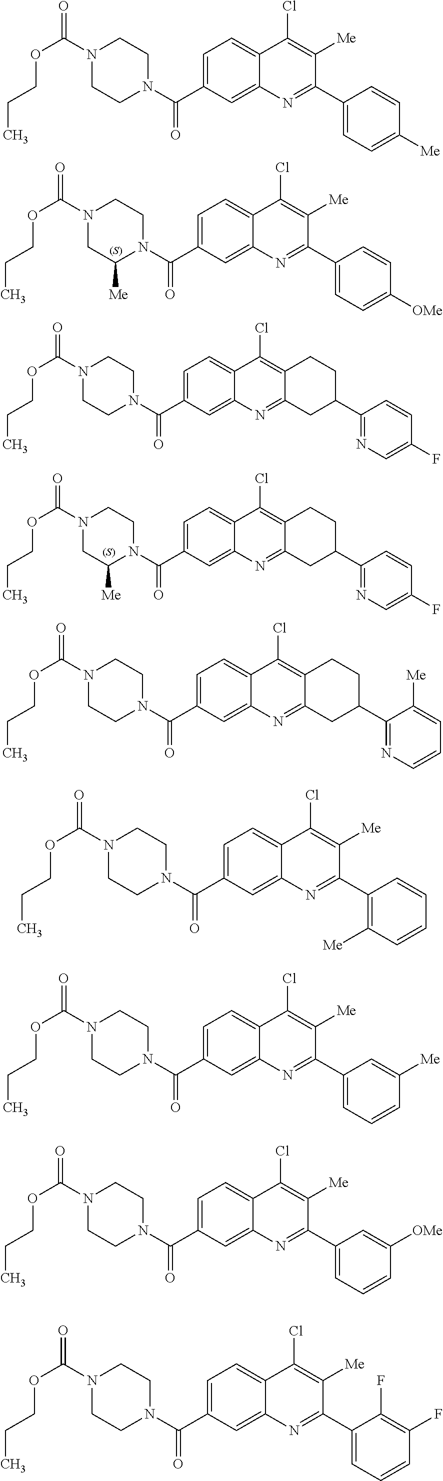 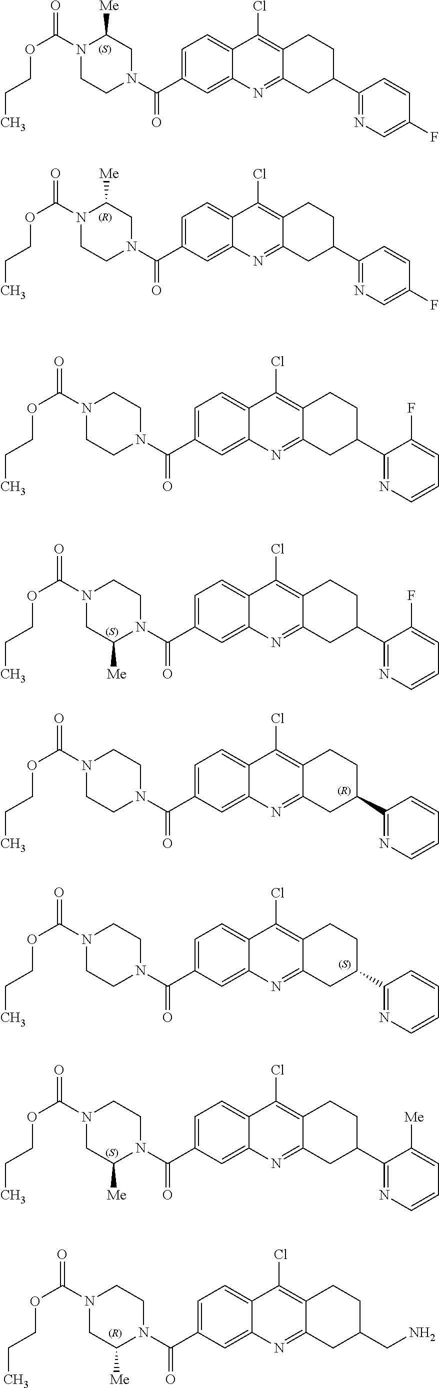 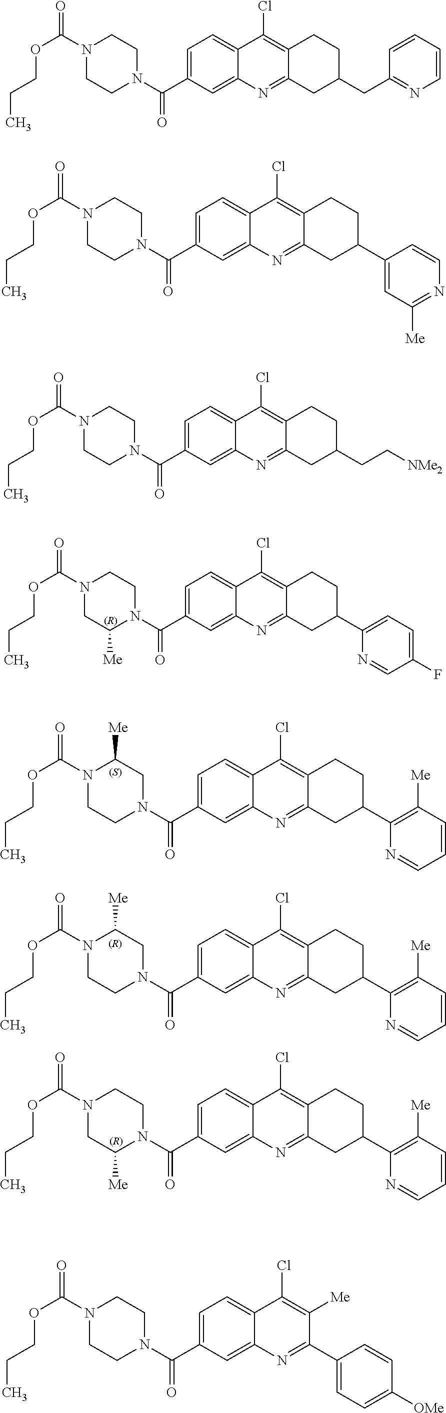 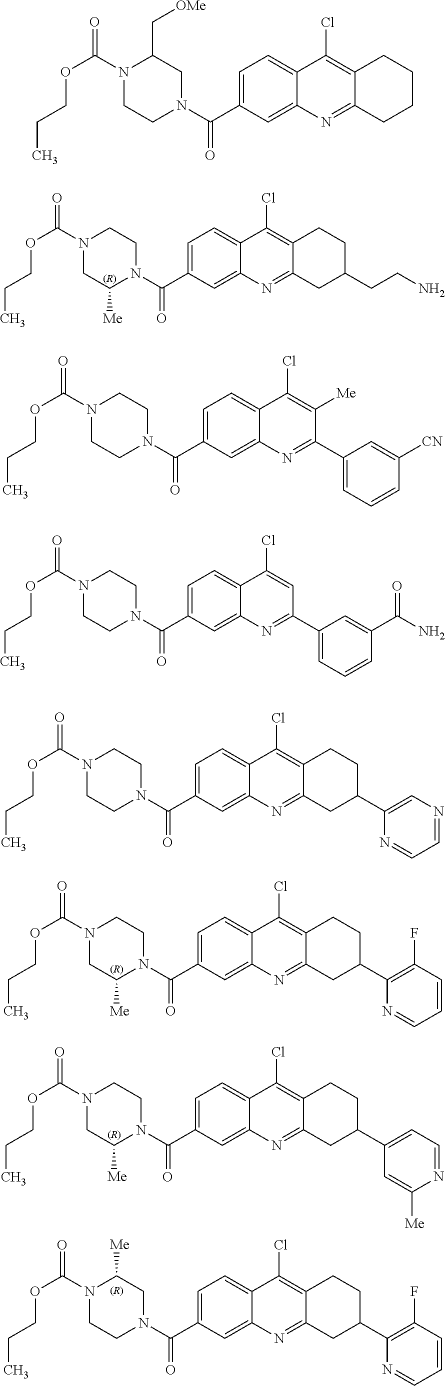 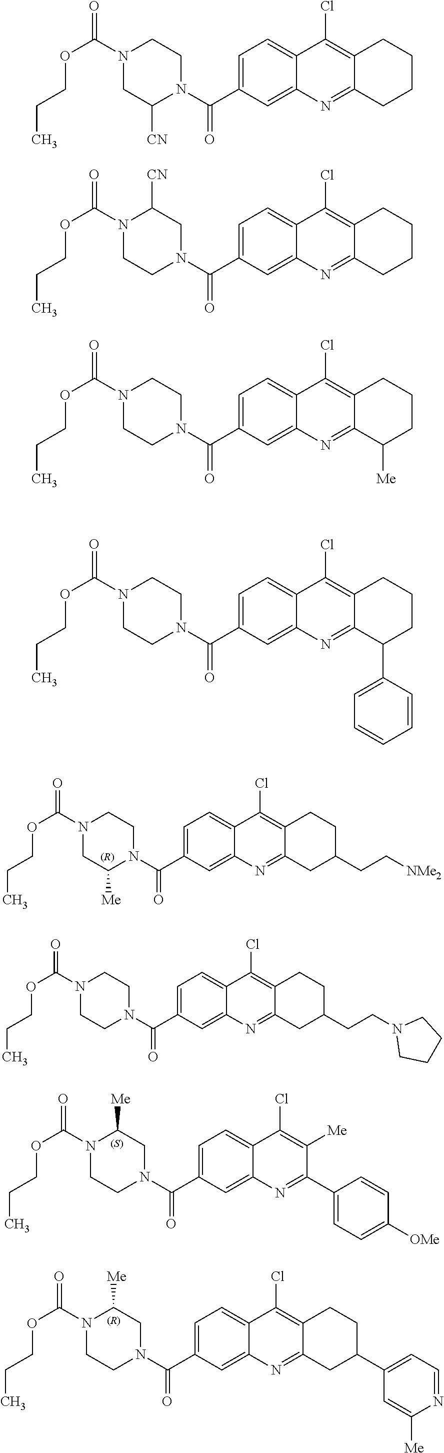 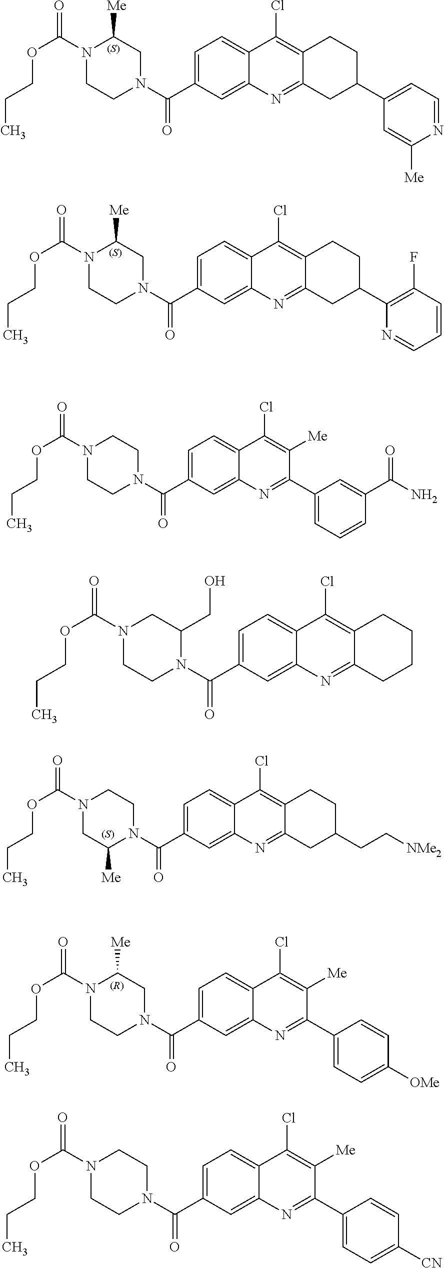 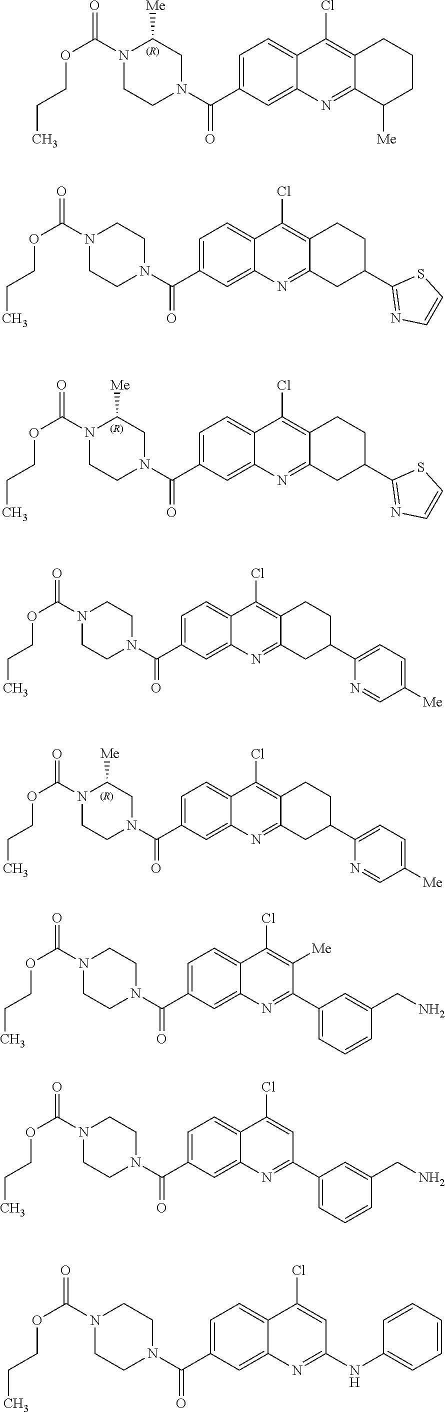 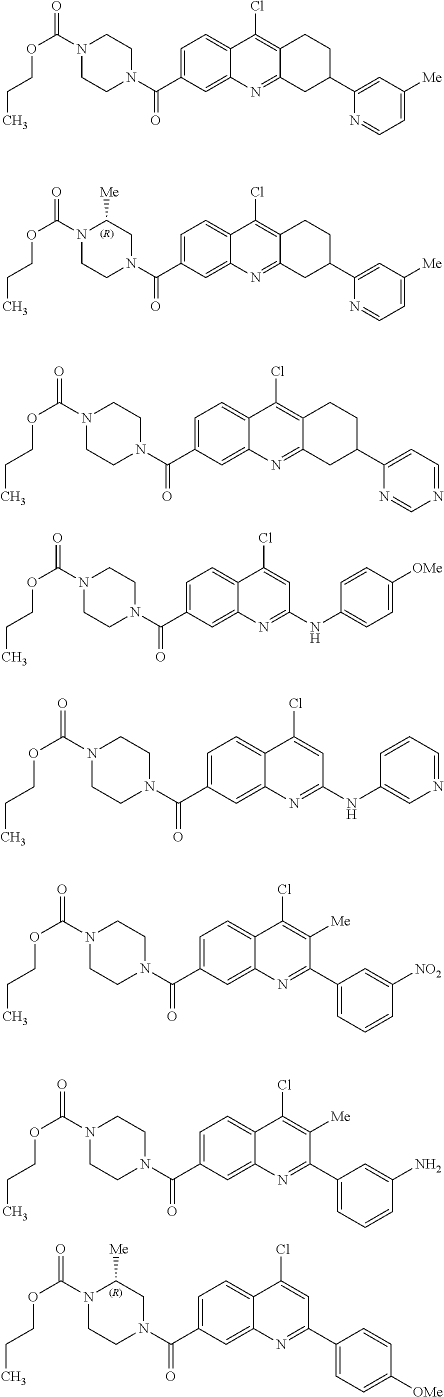 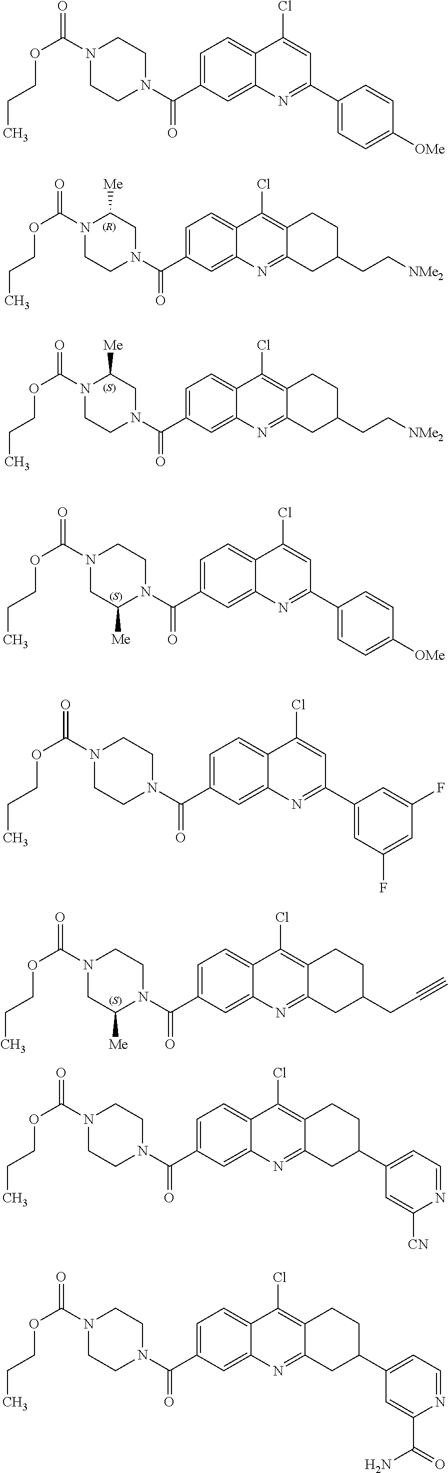 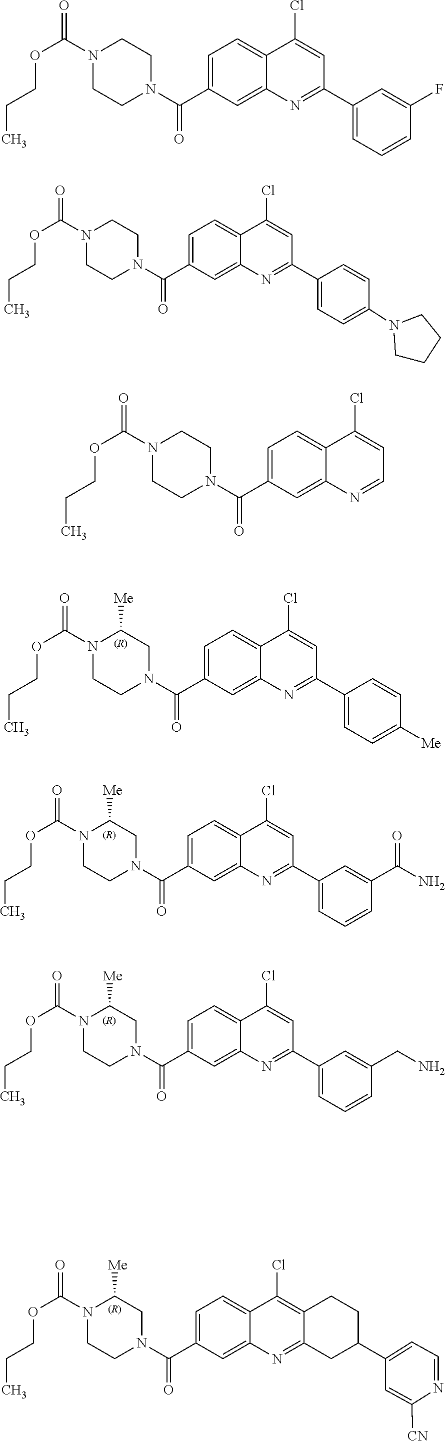 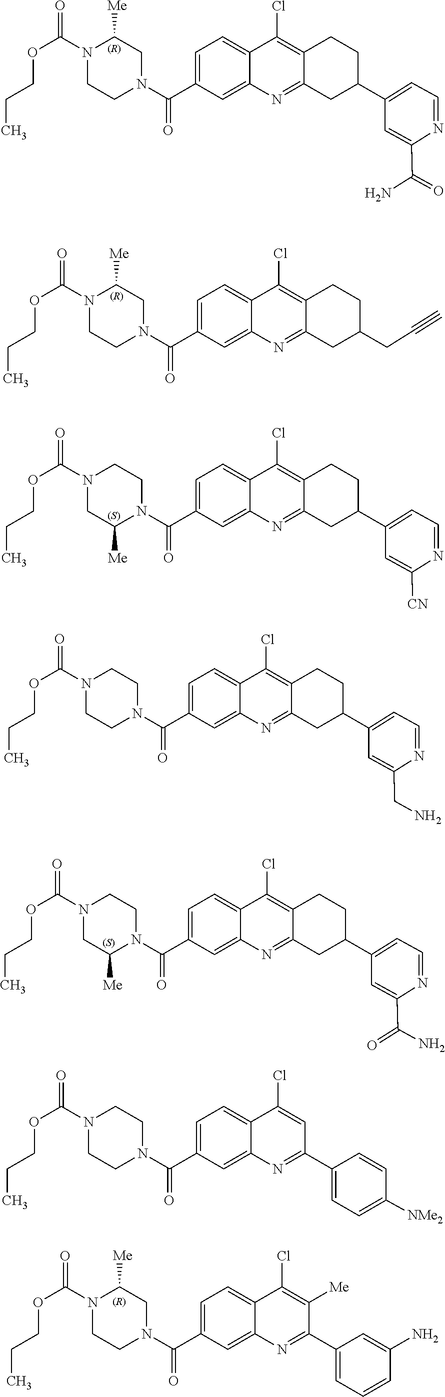 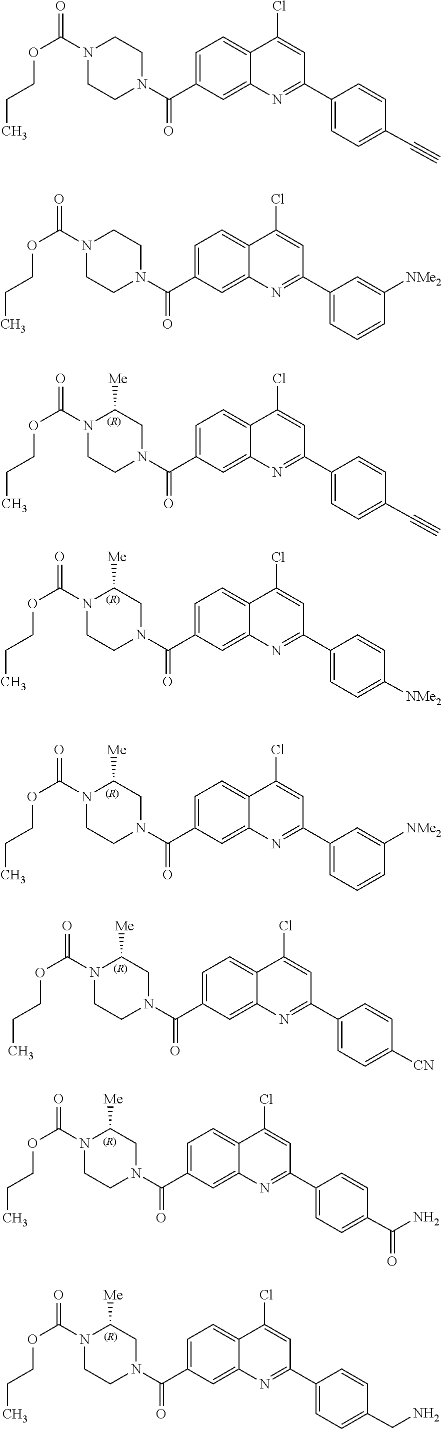 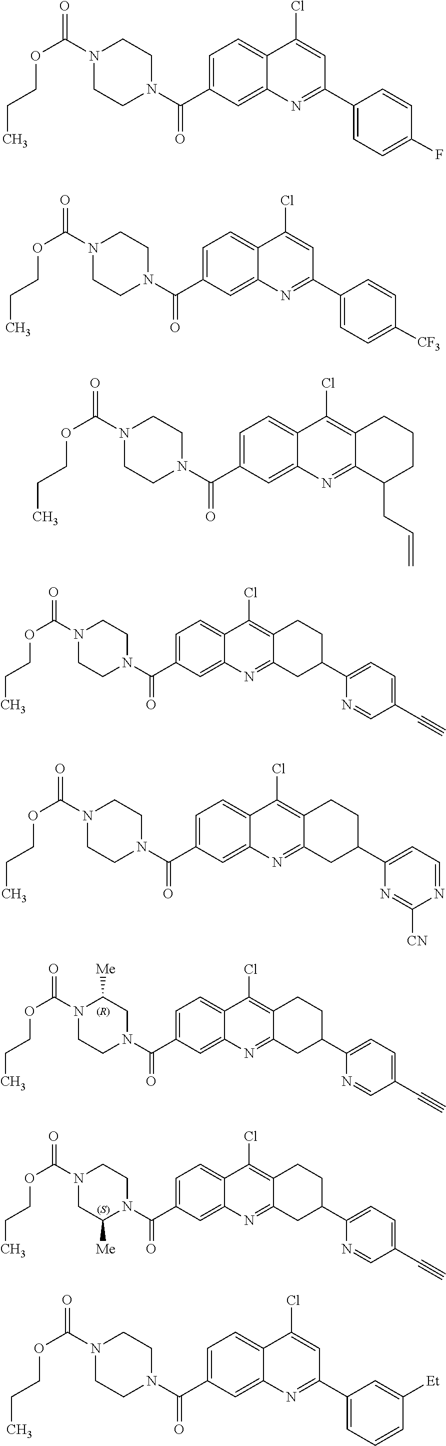 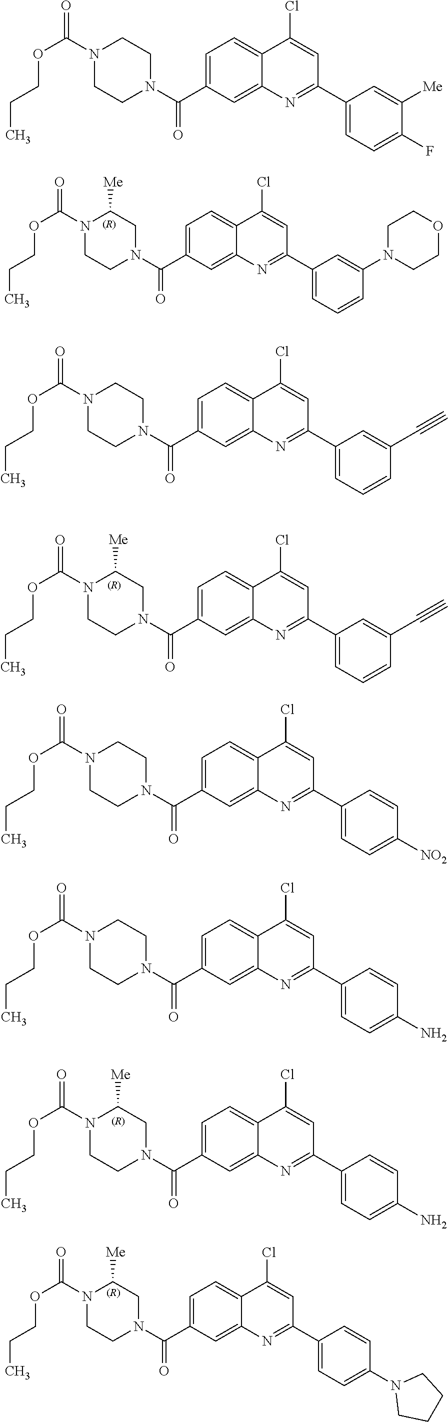 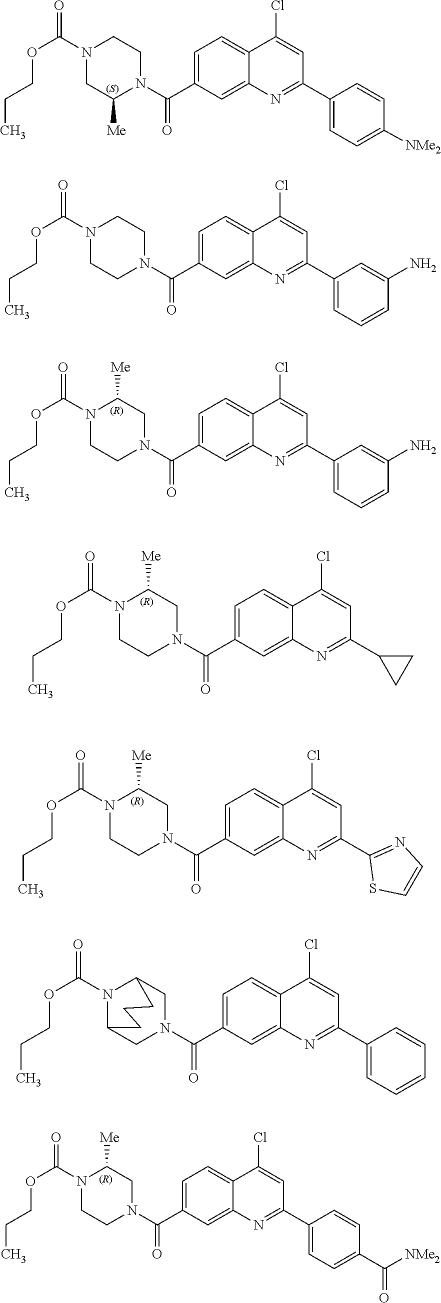 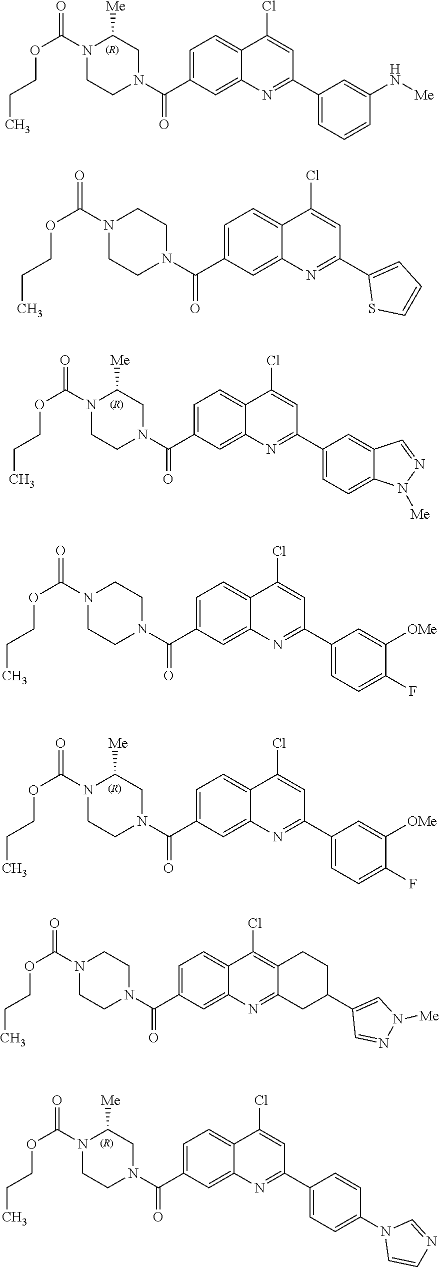 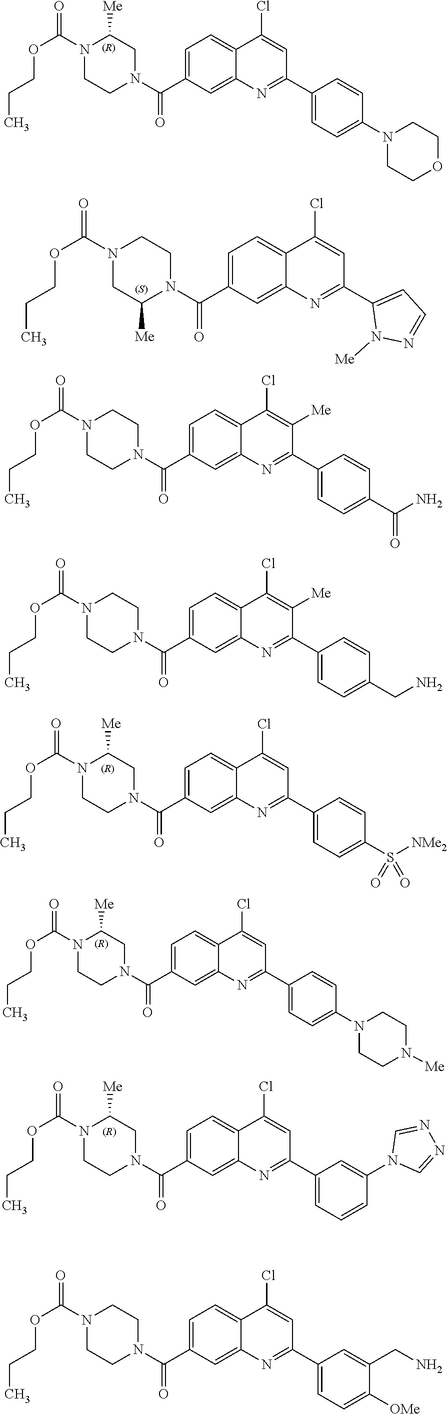 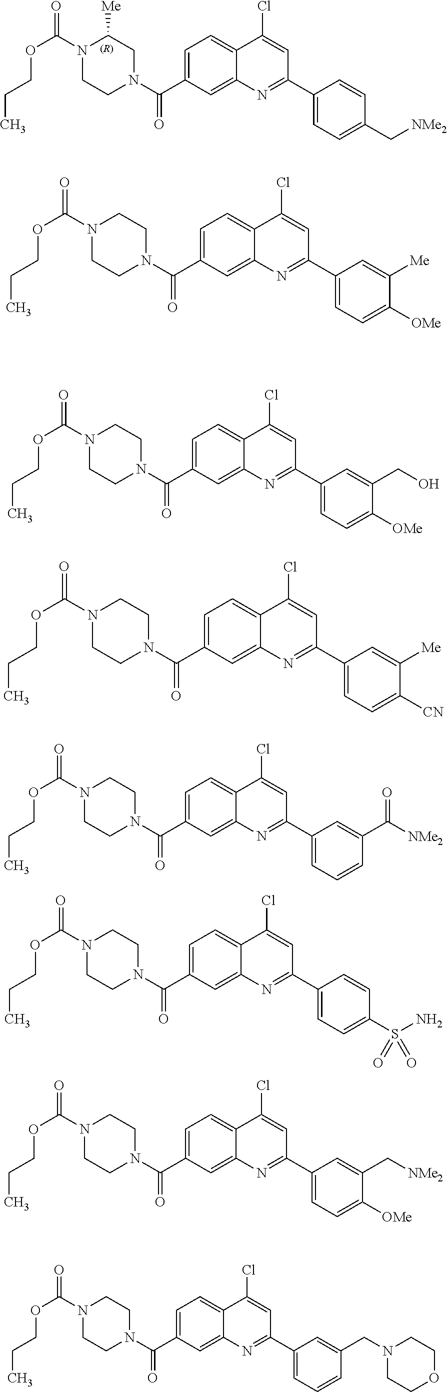 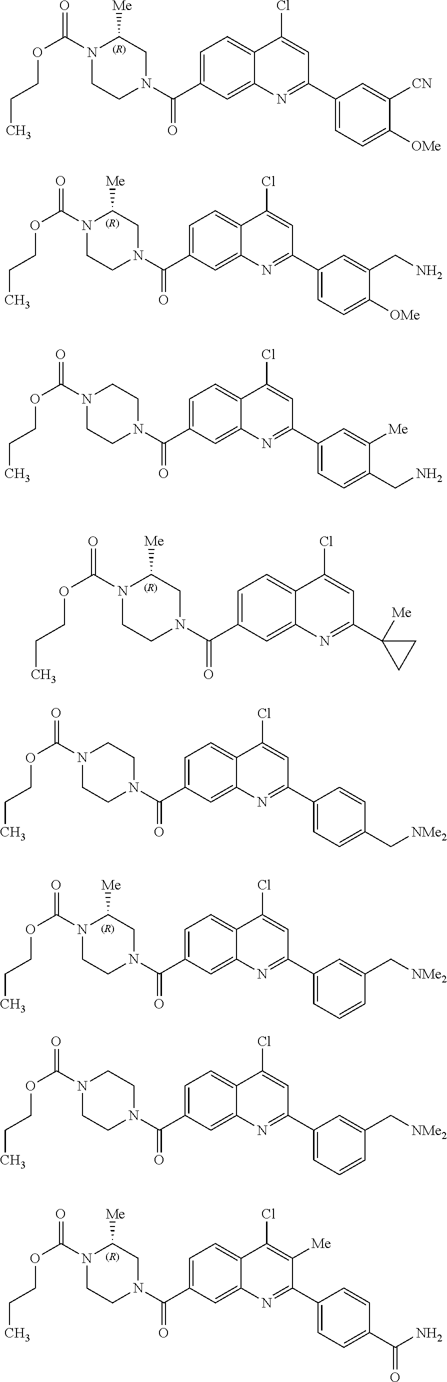 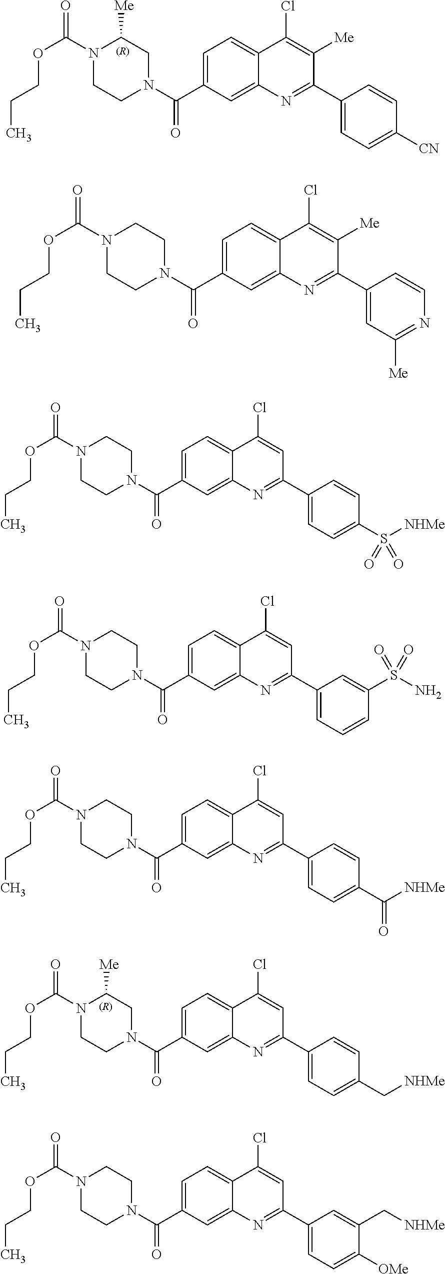 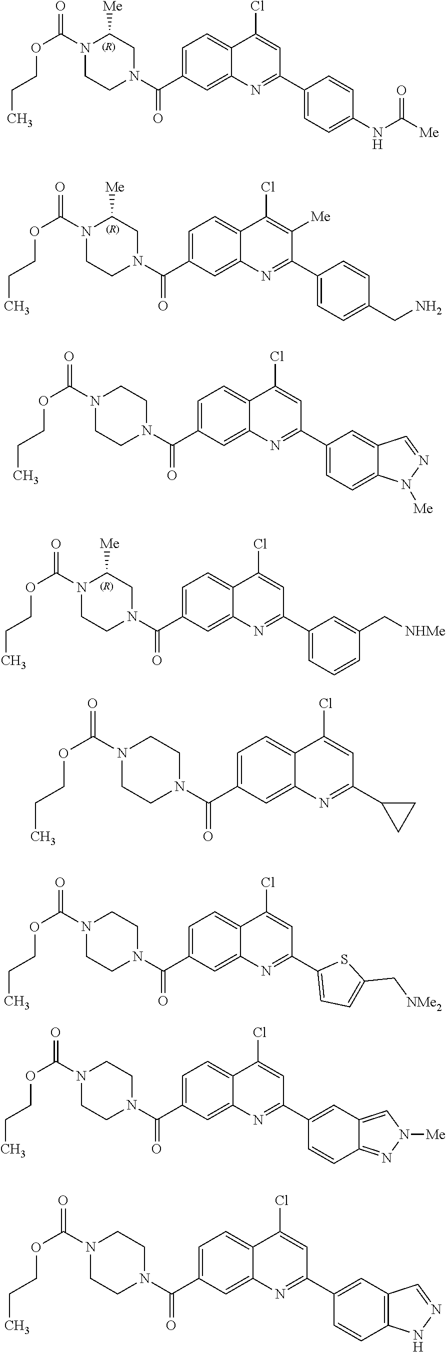 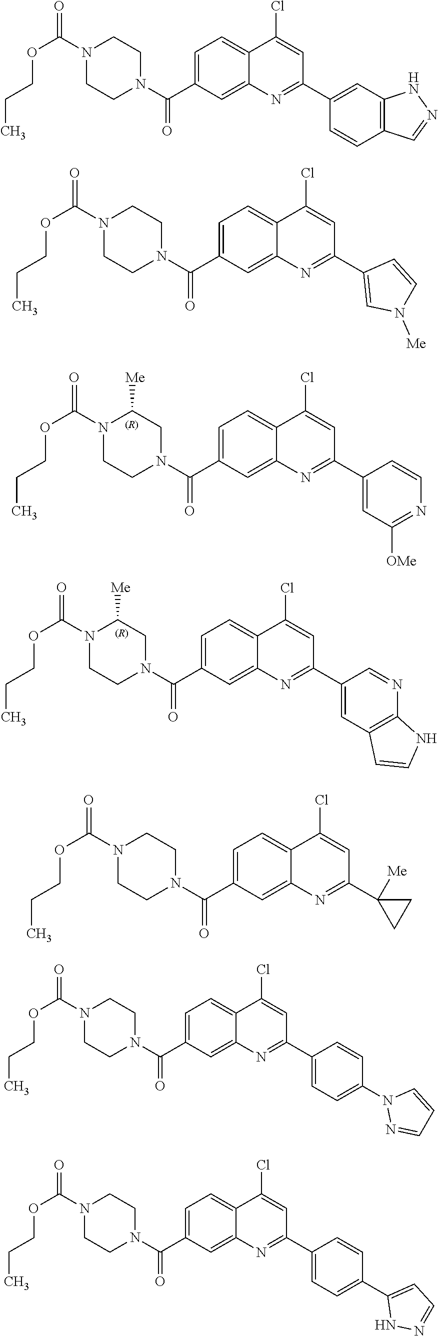 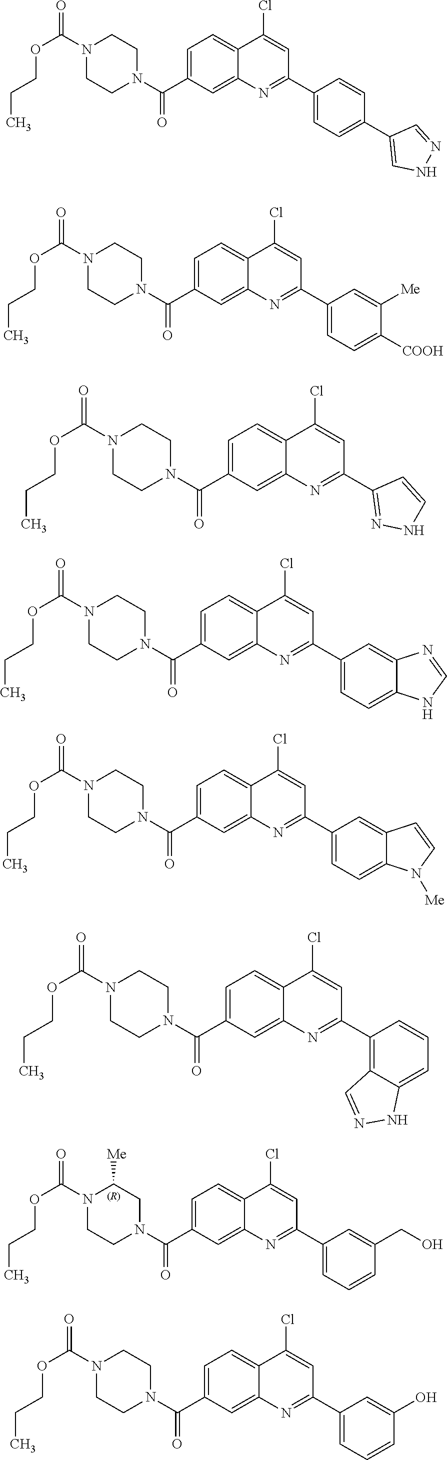 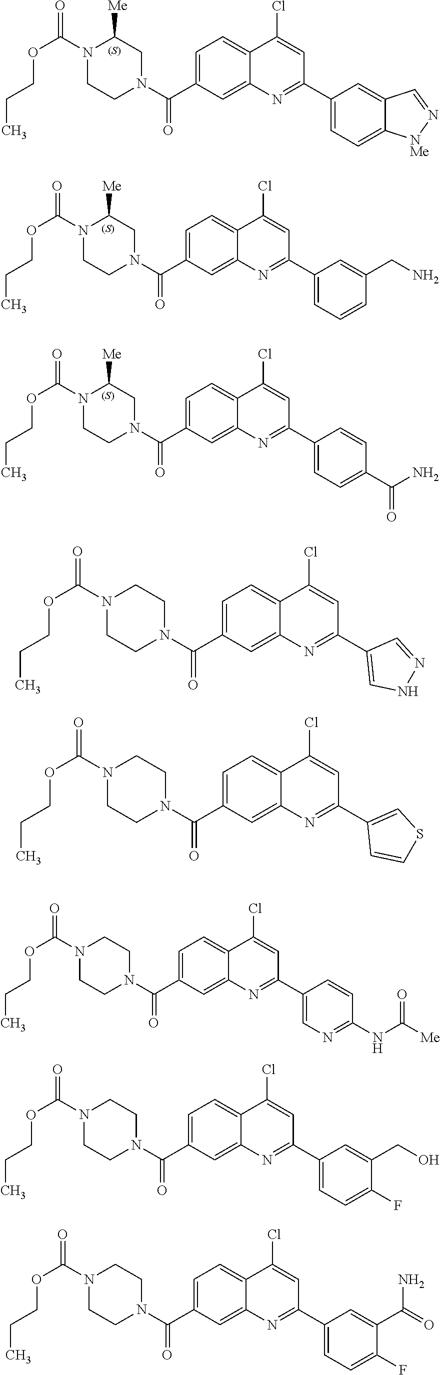 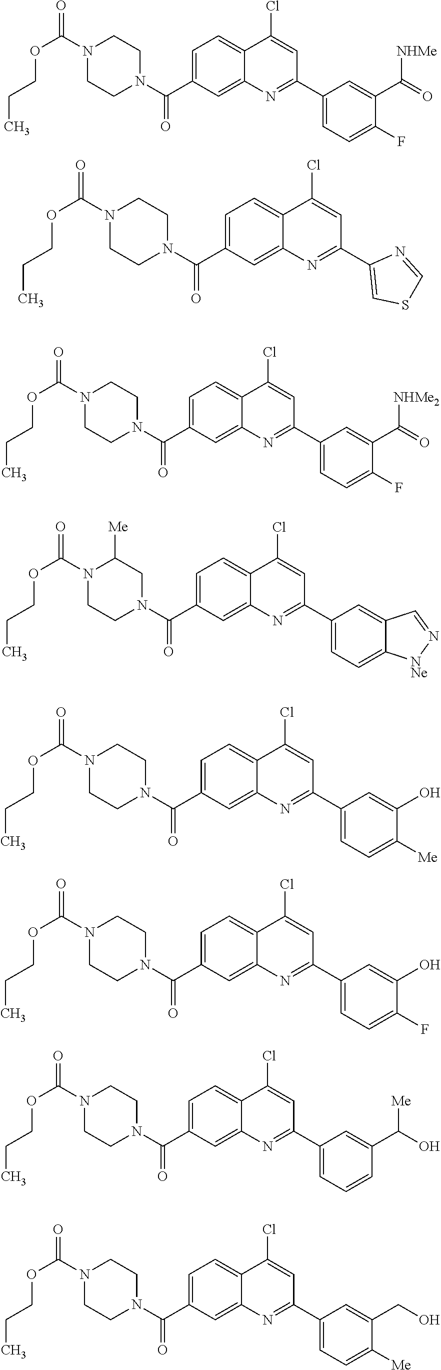 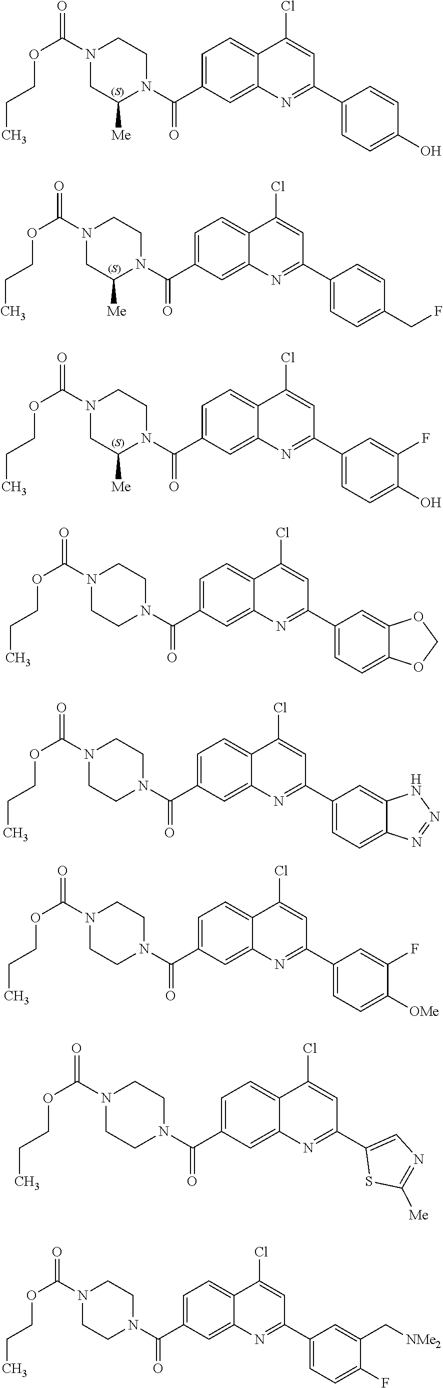 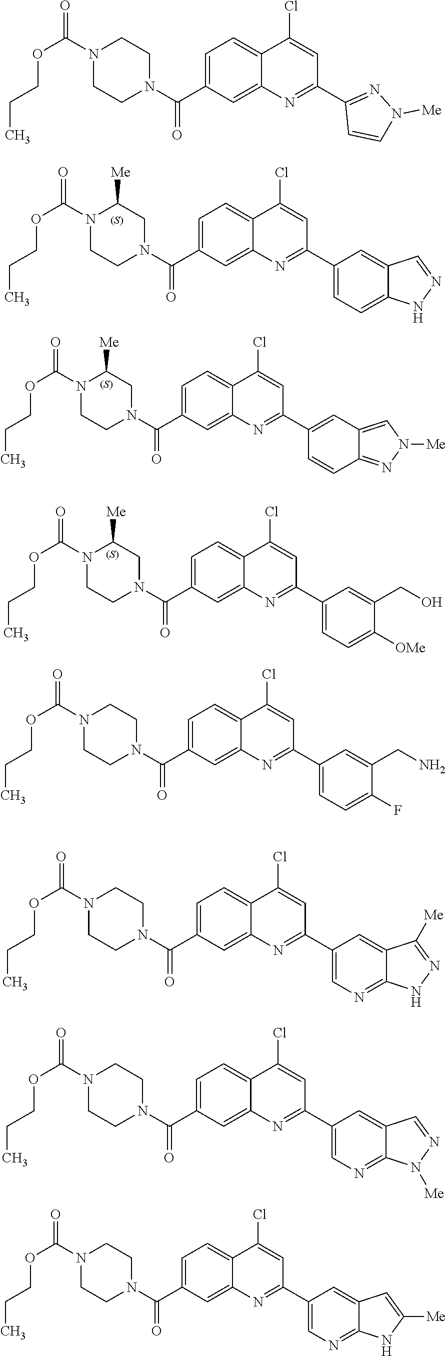 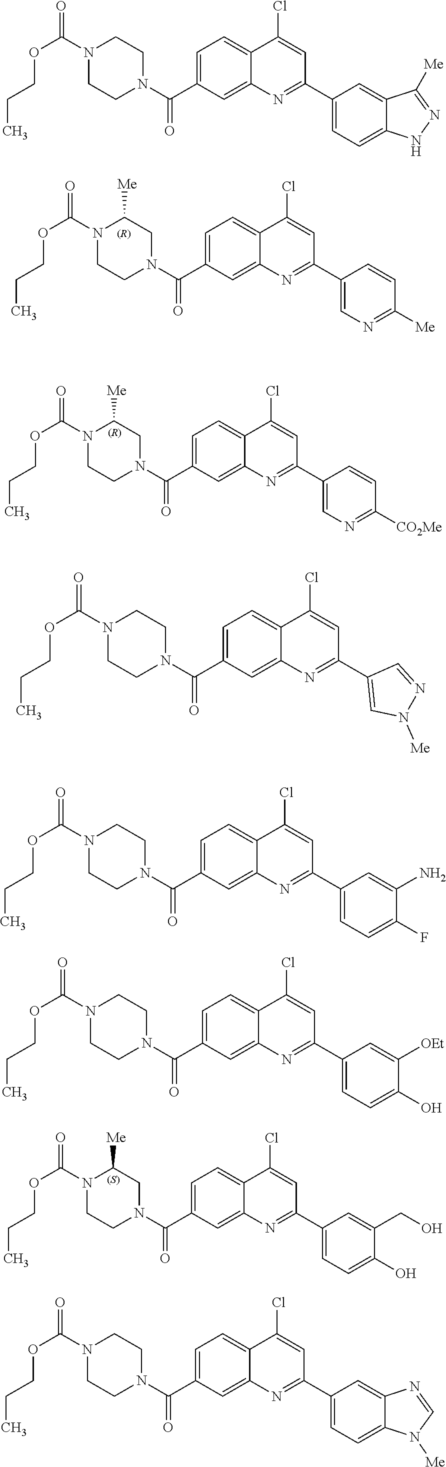 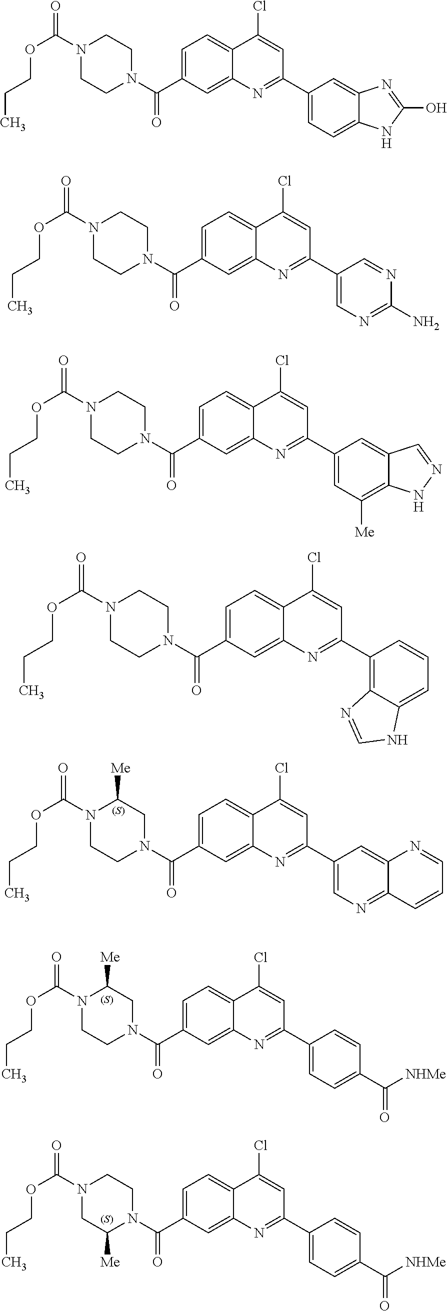 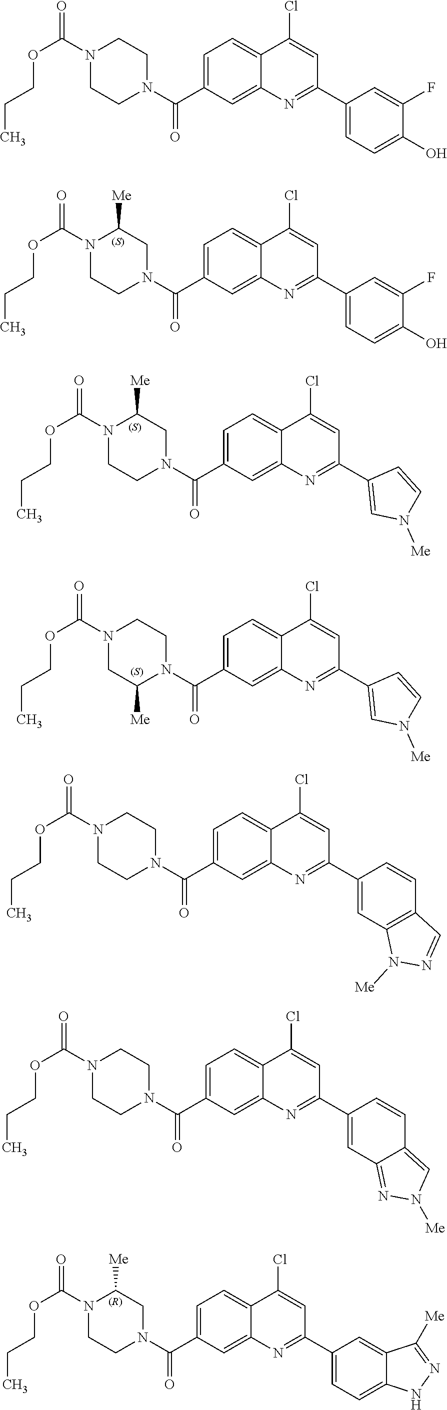 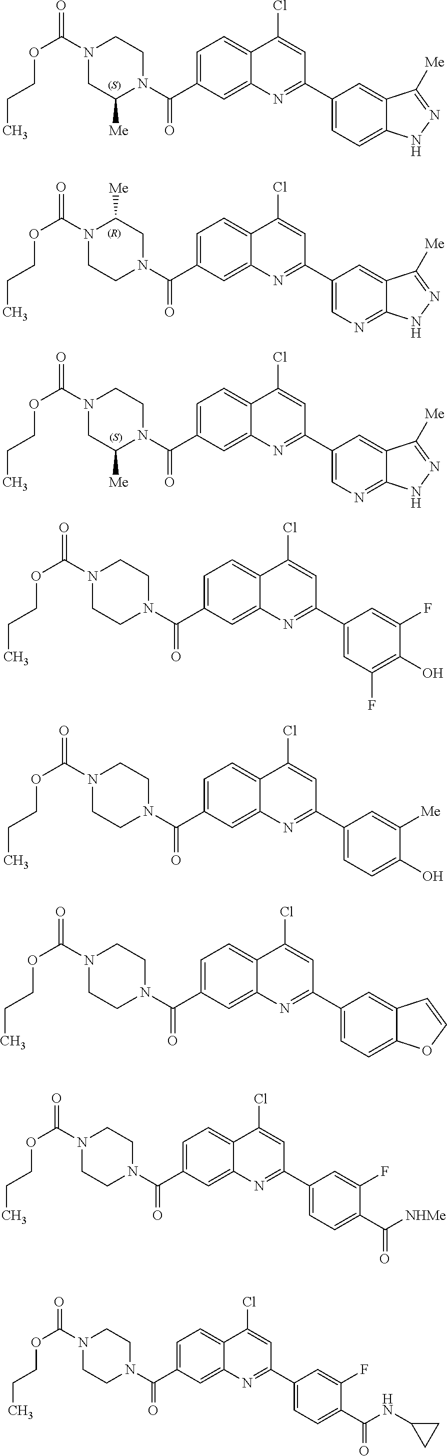 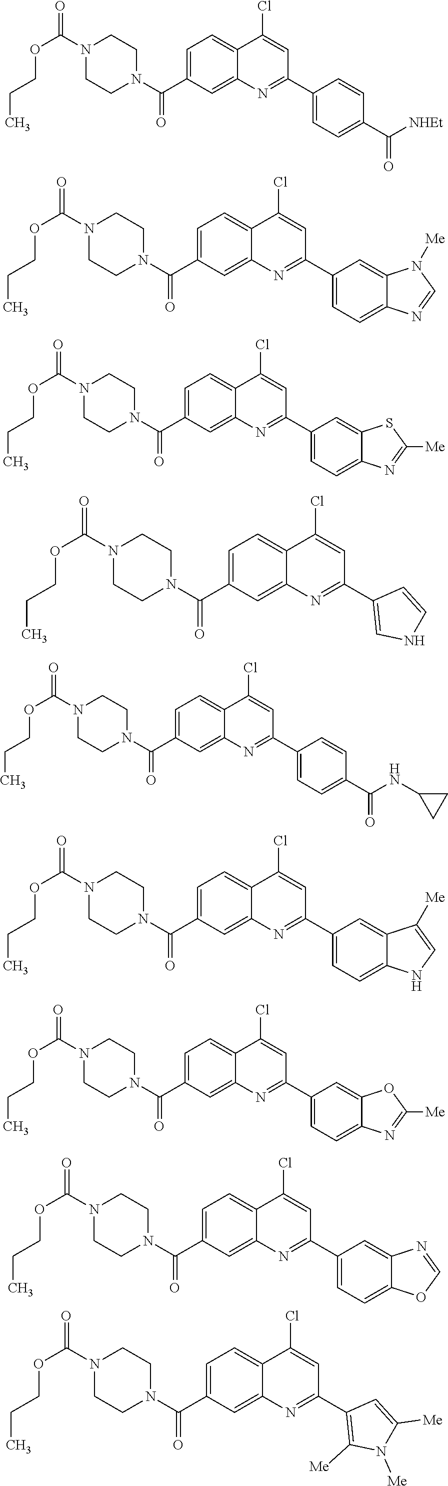 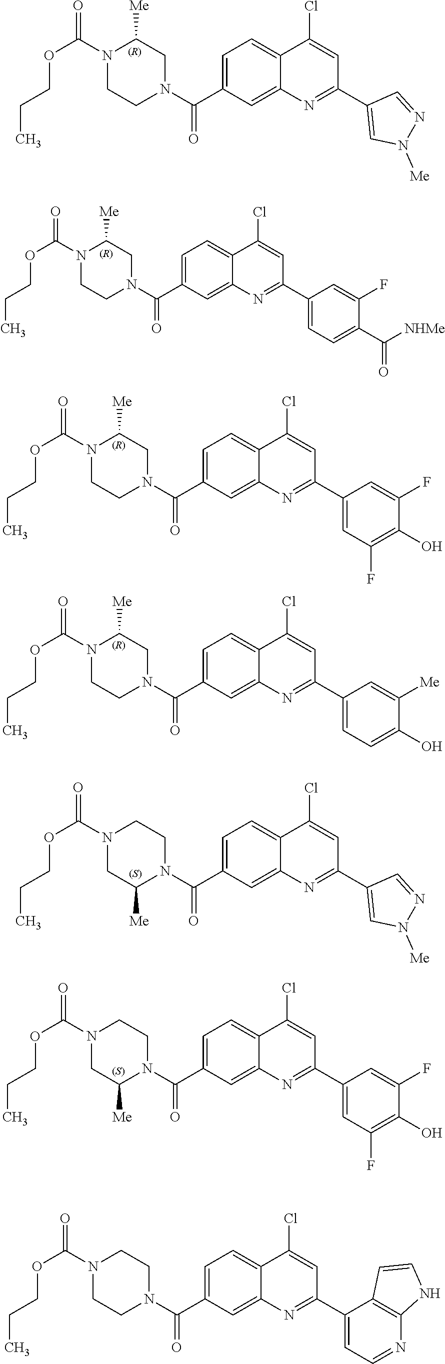 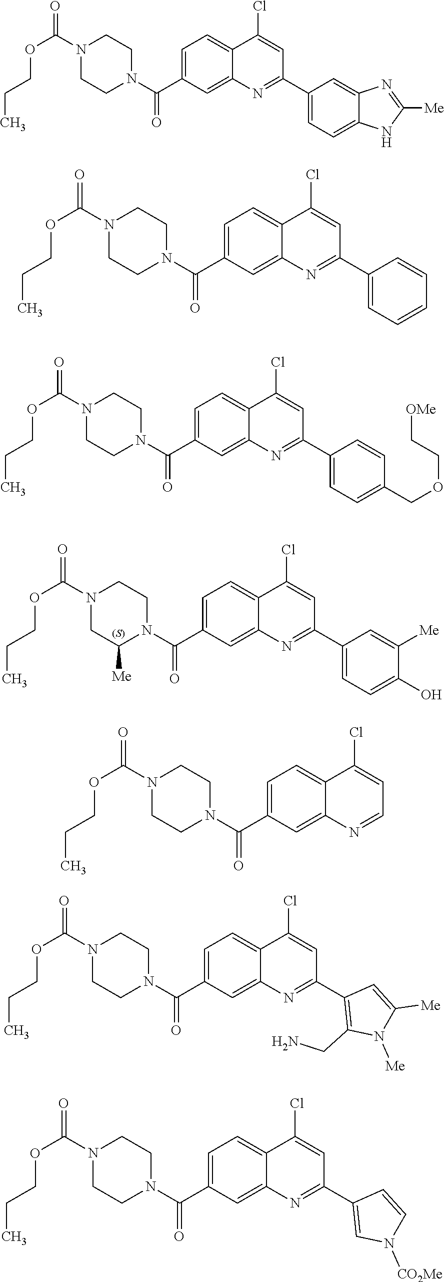 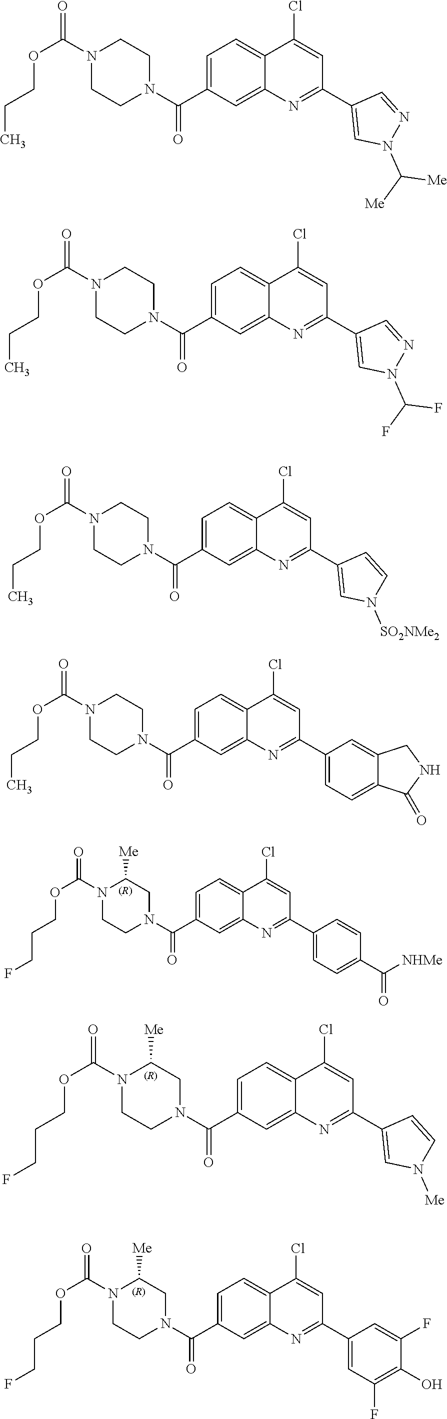 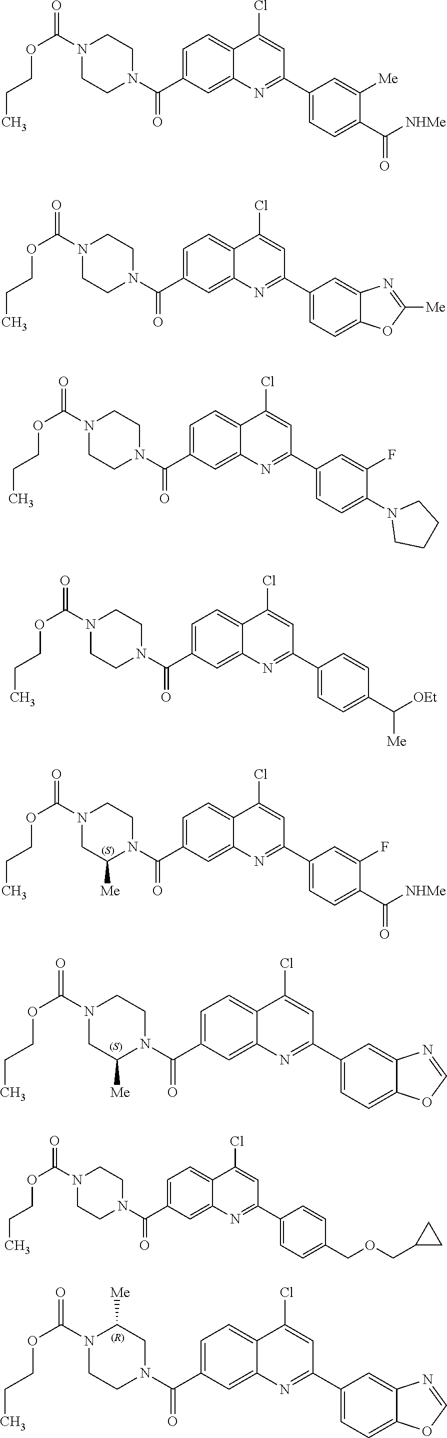 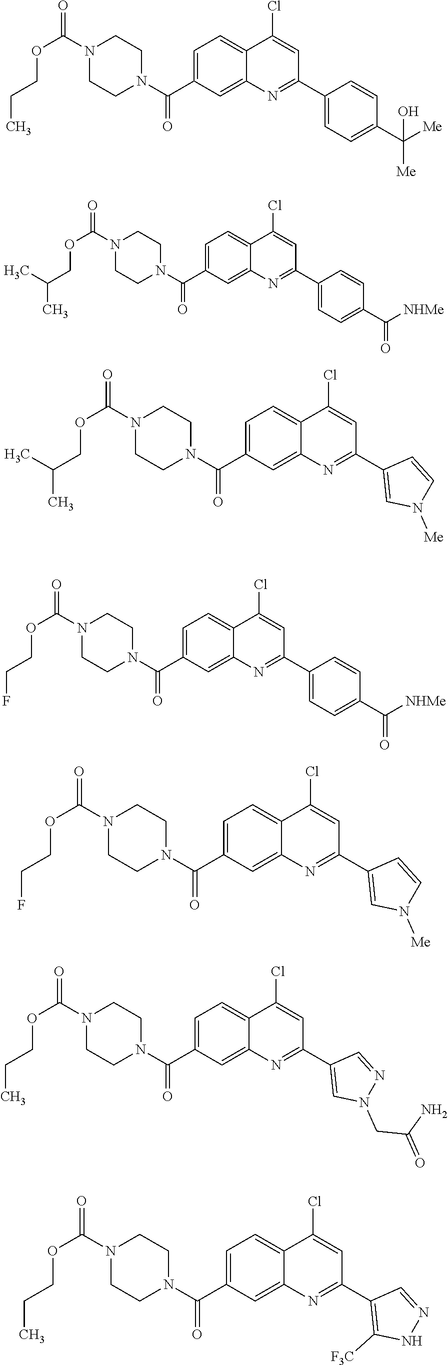 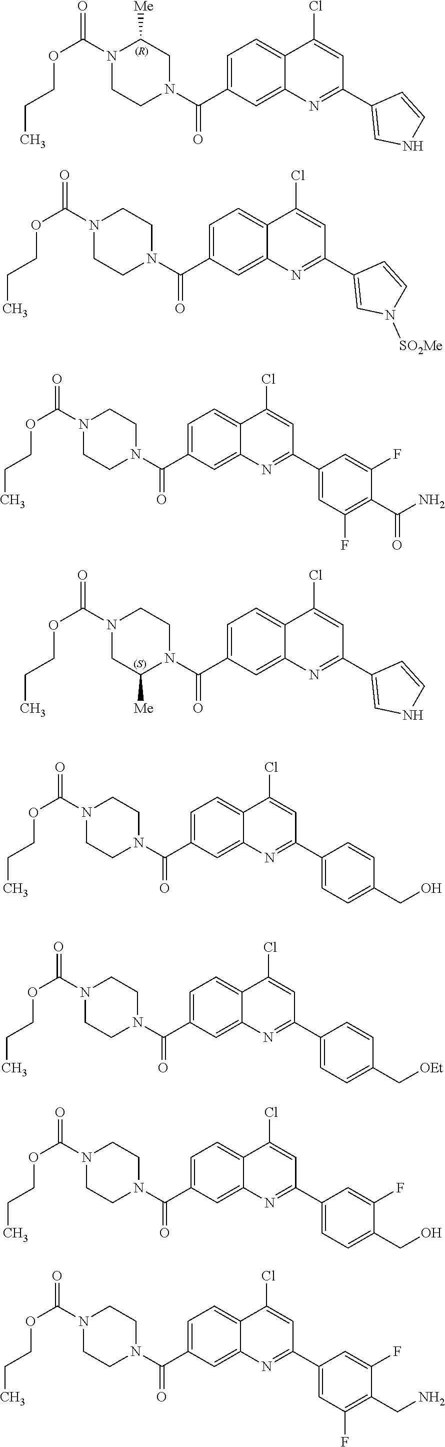 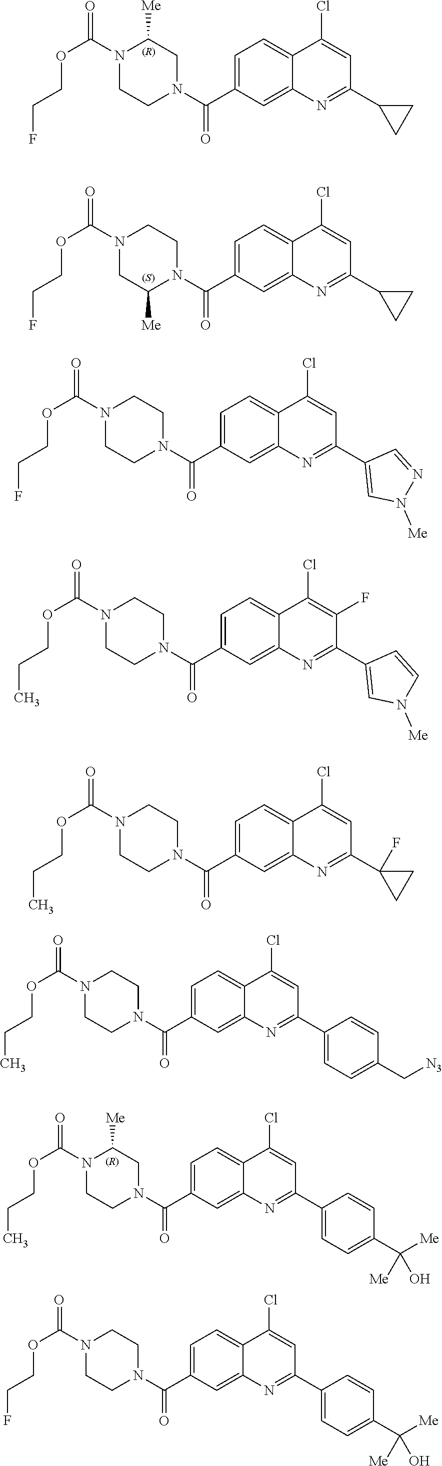 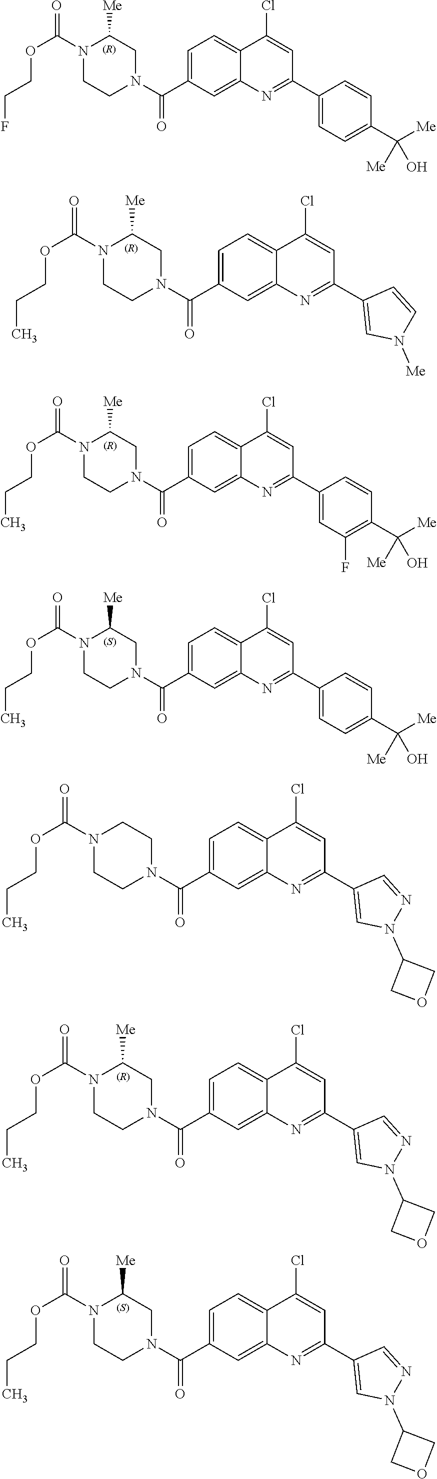 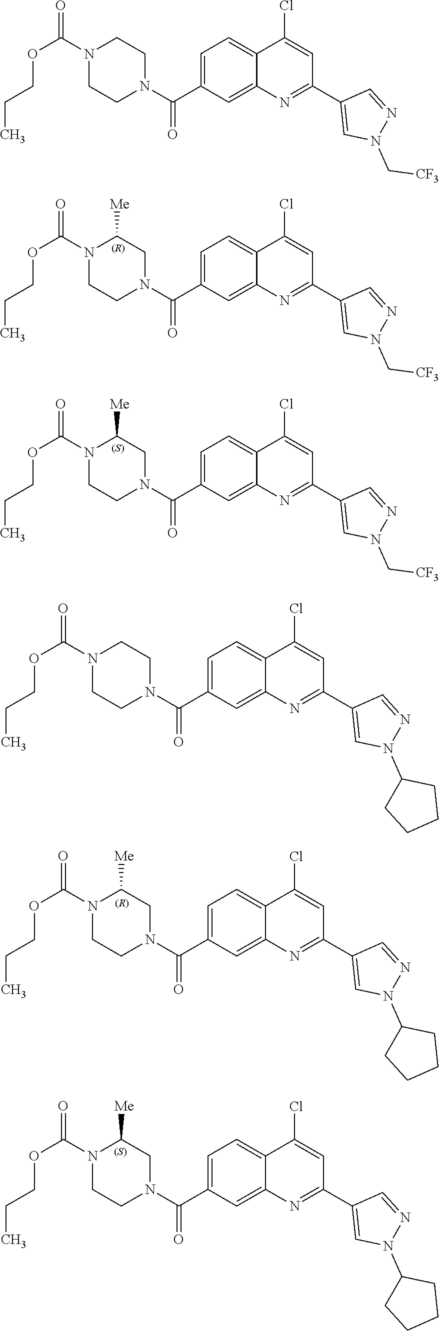 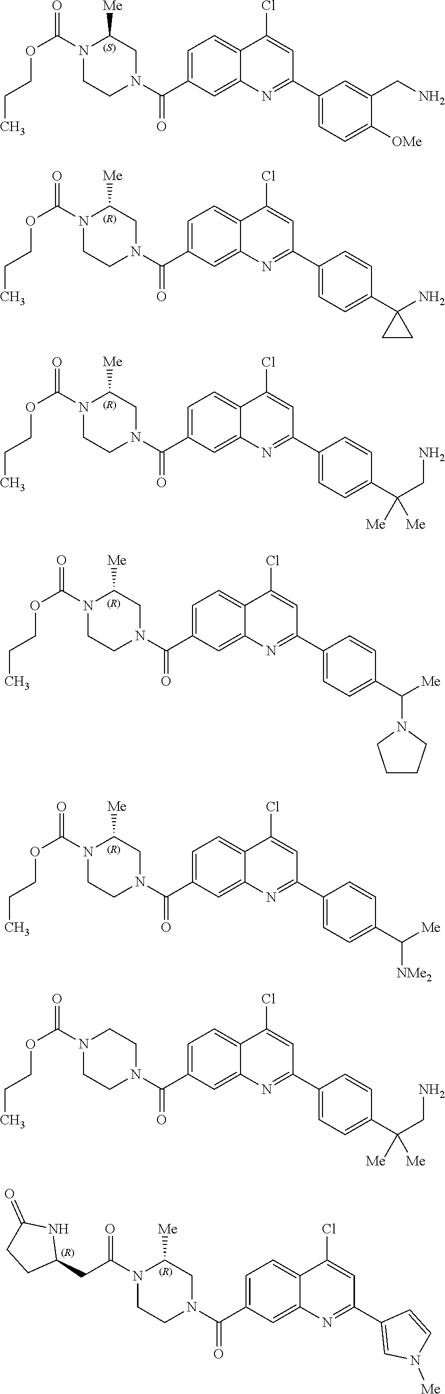 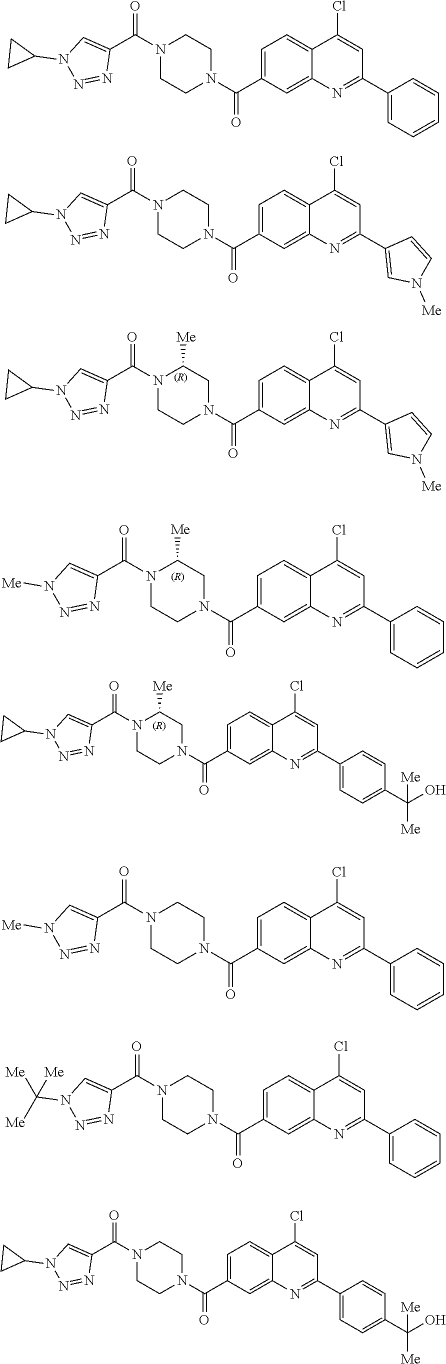 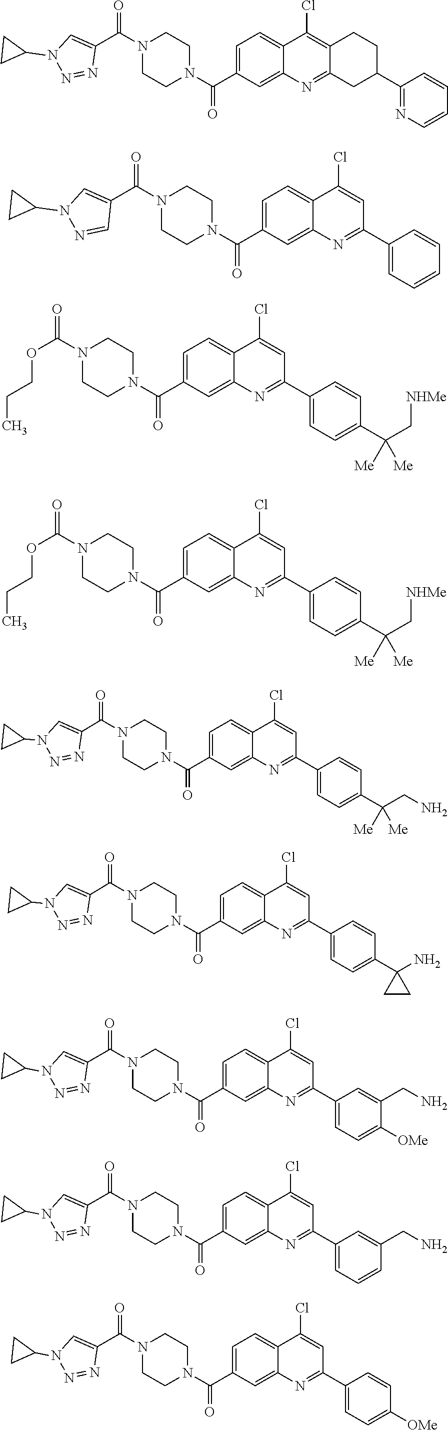 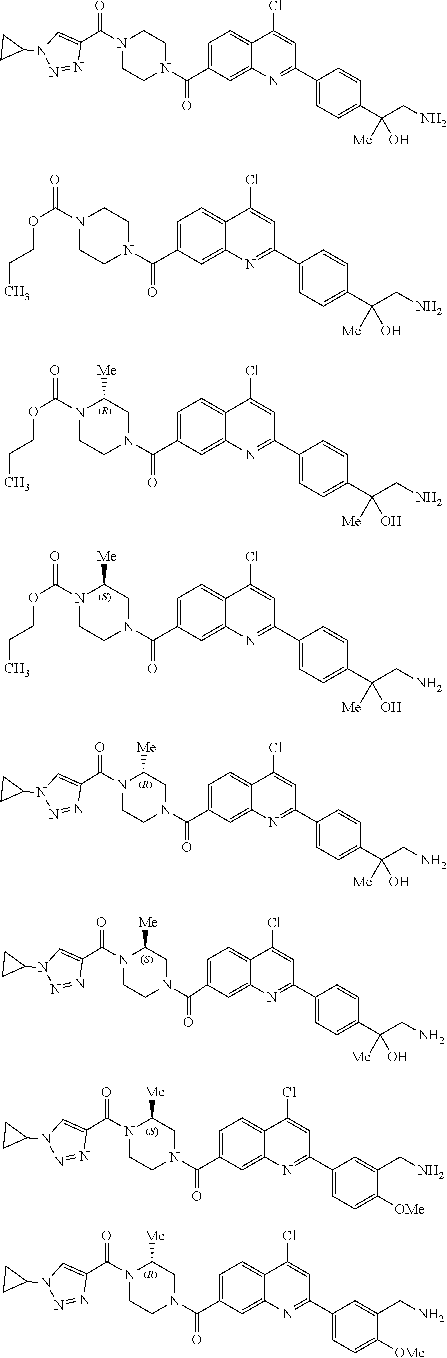 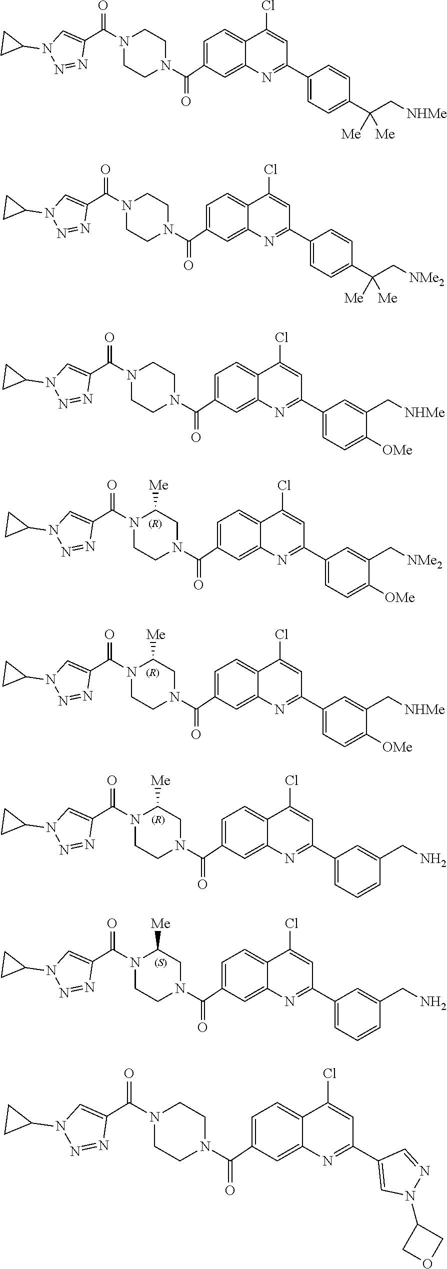 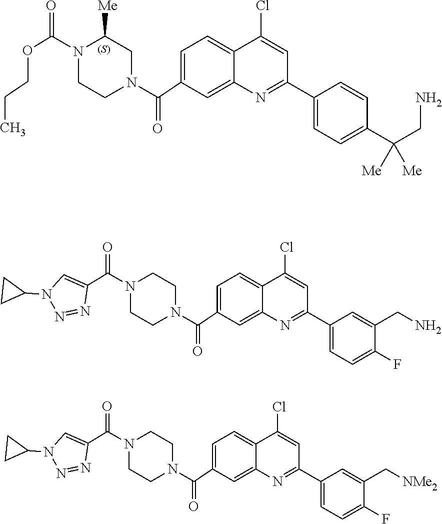
X is a halogen;
R¹ and R² are independently selected from the group consisting of a bond, H, halogen, cyano, optionally substituted alkyl, optionally substituted alkene, optionally substituted alkene, optionally substituted alkoxy, optionally substituted amino, optionally substituted cycloalkyl, optionally substituted heterocycloalkyl, optionally substituted aryl and optionally substituted heteroaryl;
and wherein R¹ and R² may optionally be taken together to form an optionally substituted alkylene bridge or an optionally substituted alkylene bridge wherein one or two alkylene units may be replaced with O, NH or 5;
and wherein R¹ and R² may optionally form an optionally substituted aryl or optionally substituted heteroaryl together with the ring atoms that they are bonded to;
R³, R⁴, R⁵, R⁶, R⁷ and R⁸ are independently absent, or selected from the group consisting of a bond, H, halogen, optionally substituted alkyl, optionally substituted alkene, optionally substituted alkyne, optionally substituted alkoxy, optionally substituted amino, optionally substituted acyl, optionally substituted cycloalkyl, optionally substituted heterocycloalkyl, optionally substituted aryl and optionally substituted heteroaryl;
wherein any two of R³, R⁴, R⁵, R⁶, R⁷ or R⁸ may be taken together to form an optionally substituted cycloalkyl, an optionally substituted alkylene bridge or an optionally substituted alkylene bridge wherein one or two alkylene units may be replaced with O, NH or 5;
Y is selected from R⁹, OR⁹ or NHR⁹, wherein R⁹ is an optionally substituted C₃ to C₁₀ alkyl, optionally substituted C₃ to C₁₀ alkenyl, optionally substituted C₃ to C₁₀ alkynyl, optionally substituted C₃ to C₇ cycloalkyl, optionally substituted C₂ to C₁₀ haloalkyl, a substituted 5-membered heteroaryl comprising two to three heteroatoms selected from N, O or S or a C₁ to C₂ alkyl substituted with an optionally substituted 5-membered heterocycloalkyl comprising one to two heteroatoms selected from N, O or S; or
a pharmaceutically acceptable form or prodrug thereof, or
a pharmaceutically acceptable form or prodrug thereof, or a pharmaceutical composition comprising a compound having the following Formula (I);
and wherein R¹ and R² may optionally form an optionally substituted aryl or optionally substituted heteroaryl together with the ring atoms that they are bonded to;
R³, R⁴, R⁵, R⁶, R⁷ and R⁸ are independently absent, or selected from the group consisting of a bond, H, halogen, optionally substituted alkyl, optionally substituted alkene, optionally substituted alkene, optionally substituted alkoxy, optionally substituted amino, optionally substituted acyl, optionally substituted cycloalkyl, optionally substituted heterocycloalkyl, optionally substituted aryl and optionally substituted heteroaryl;
wherein any two of R³, R⁴, R⁵, R⁶, R⁷ or R⁸ may be taken together to form an optionally substituted cycloalkyl, an optionally substituted alkylene bridge or an optionally substituted alkylene bridge wherein one or two alkylene units may be replaced with O, NH or S;
Y is selected from R⁹, OR⁹ or NHR⁹, wherein R⁹ is an optionally substituted C₃ to C₁₀ alkyl, optionally substituted C₃ to C₁₀ alkenyl, optionally substituted C₃ to C₁₀ alkynyl, optionally substituted C₃ to C₇ cycloalkyl, optionally substituted C₂ to C₁₀ haloalkyl, a substituted 5-membered heteroaryl comprising two to three heteroatoms selected from N, O or S or a C₁ to C₂ alkyl substituted with an optionally substituted 5-membered heterocycloalkyl comprising one to two heteroatoms selected from N, O or S;
or a pharmaceutically acceptable form or prodrug thereof, or a pharmaceutically acceptable form or prodrug thereof, and a pharmaceutically acceptable excipient.

37.-44. (canceled)

45. A method of treating a SMYD-3-related disorder comprising administering to a subject in need of treatment a compound having the following Formula (I); 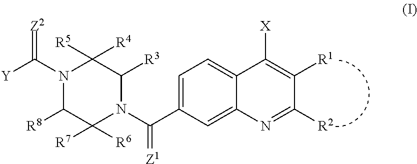
wherein
Z¹ and Z² are independently selected from O, S or NH;
or a pharmaceutically acceptable form or prodrug thereof.

27. The compound claim 1, wherein the compound is an enzyme inhibitor, is a protein lysine methyltransferase (PKMT) inhibitor,
wherein the protein lysine methyltransferase is SMYD3, inhibits methylation of histone inhibits the trimethylation of histone H3 at lysine 4 (H3K4me3) and/or methylation of histone H4 at lysine 5 (H4K5me),
modulates moystatin transcription and/or c-Met transcription,
modulates the MEK-ERK mitogen-activated protein-kinase pathway, or
inhibits methylation of a lysine residue on MAP3K2, or
wherein the lysine residue is K260.

28.-35. (canceled)

36. A pharmaceutical composition comprising a compound having the following Formula (I); 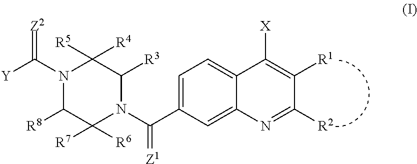
wherein
Z¹ and Z² are independently selected from O, S or NH;
X is a halogen;
R¹ and R² are independently selected from the group consisting of a bond, H, halogen, cyano, optionally substituted alkyl, optionally substituted alkene, optionally substituted alkyne, optionally substituted alkoxy, optionally substituted amino, optionally substituted cycloalkyl, optionally substituted heterocycloalkyl, optionally substituted aryl and optionally substituted heteroaryl;
and wherein R¹ and R² may optionally be taken together to form an optionally substituted alkylene bridge or an optionally substituted alkylene bridge wherein one or two alkylene units may be replaced with O, NH or S; 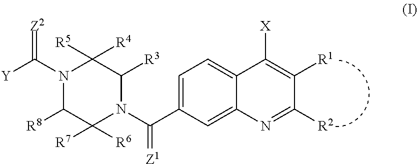
wherein
Z¹ and Z² are independently selected from O, S or NH;
X is a halogen;
R¹ and R² are independently selected from the group consisting of a bond, H, halogen, cyano, optionally substituted alkyl, optionally substituted alkene, optionally substituted alkyne, optionally substituted alkoxy, optionally substituted amino, optionally substituted cycloalkyl, optionally substituted heterocycloalkyl, optionally substituted aryl and optionally substituted heteroaryl;
and wherein R¹ and R² may optionally be taken together to form an optionally substituted alkylene bridge or an optionally substituted alkylene bridge wherein one or two alkylene units may be replaced with O, NH or S;
and wherein R¹ and R² may optionally form an optionally substituted aryl or optionally substituted heteroaryl together with the ring atoms that they are bonded to;
R³, R⁴, R⁵, R⁶, R⁷ and R⁸ are independently absent, or selected from the group consisting of a bond, H, halogen, optionally substituted alkyl, optionally substituted alkene, optionally substituted alkyne, optionally substituted alkoxy, optionally substituted amino, optionally substituted acyl, optionally substituted cycloalkyl, optionally substituted heterocycloalkyl, optionally substituted aryl and optionally substituted heteroaryl;
wherein any two of R³, R⁴, R⁵, R⁶, R⁷ or R⁸ may be taken together to form an optionally substituted cycloalkyl, an optionally substituted alkylene bridge or an optionally substituted alkylene bridge wherein one or two alkylene units may be replaced with O, NH or S;
Y is selected from R⁹, OR⁹ or NHR⁹, wherein R⁹ is an optionally substituted C₃ to C₁₀ alkyl, optionally substituted C₃ to C₁₀ alkenyl, optionally substituted C₃ to C₁₀ alkynyl, optionally substituted C₃ to C₇ cycloalkyl, optionally substituted C₂ to C₁₀ haloalkyl, a substituted 5-membered heteroaryl comprising two to three heteroatoms selected from N, O or S or a C₁ to C₂ alkyl substituted with an optionally substituted 5-membered heterocycloalkyl comprising one to two heteroatoms selected from N, O or S; or
a pharmaceutically acceptable form or prodrug thereof, or
a pharmaceutically acceptable form or prodrug thereof, and
a pharmaceutically acceptable excipient.

46. The method according to claim 45, wherein the disorder is cancer, angiogenic disorder or pathological angiogenesis, fibrosis or inflammatory conditions, or
the disorder is lymphoma, cutaneous T-cell lymphoma, follicular lymphoma, or Hodgkin lymphoma, cervical cancer, ovarian cancer, breast cancer, lung cancer, prostate cancer, colorectal cancer, gastric cancer, pancreatic cancer, sarcoma, hepatocellular carcinoma, leukemia or myeloma, retinal angiogenic disease, liver fibrosis, kidney fibrosis, or myelofibrosis.

47.-61. (canceled)
